(19) United States
(12) Patent Application Publication
(10) Pub. No.: US 2016/0222014 A1
(43) Pub. Date: Aug. 4, 2016

(54) COMPOUNDS FOR REGULATING FAK AND/OR SRC PATHWAYS

(71) Applicant: ASANA BIOSCIENCES, LLC, Bridgewater, NJ (US)

(72) Inventors: Aranapakam M. Venkatesan, Malvern, PA (US); Roger A. Smith, Chester Springs, PA (US); Scott K. Thompson, Phoenixville, PA (US); Nicholas Laping, West Chester, PA (US); Bheemashankar Kulkarni, Pune (IN); Gurulingappa Hallur, Bangalore (IN); Vellarkad N. Viswanadhan, Bangalore (IN); Muralidhar Pendyala, Gopavaram, Gopalapuram mandal (IN); Raghava Reddy Kethiri, Bangalore (IN); Rajiv Tyagi, Bangalore (IN); Dhanalakshmi Sivanandhan, Bangalore (IN); Rajagopal Bakthavatchalam, Madison, CT (US)

(73) Assignee: Asana Biosciences, LLC, Bridgewater, NJ (US)

(21) Appl. No.: 15/021,186

(22) PCT Filed: Sep. 5, 2014
(86) PCT No.: PCT/US2014/054197

§ 371 Date: Mar. 10, 2016

Related U.S. Application Data
(60) Provisional application No. 61/875,963, filed on Sep. 10, 2013.

(51) Int. Cl. C07D 487/04; A61K 45/06; C07D 471/04; A61K 31/444; A61K 31/4709; A61N 5/10; A61K 31/5377; A61K 31/538; A61K 31/496; C07D 519/00; A61K 31/4545; A61K 31/551; A61K 31/519; A61K 31/437

(52) U.S. Cl. CPC C07D 487/04; A61K 31/519; A61K 45/06; C07D 471/04; A61K 31/444; A61K 31/4709; A61K 31/437; A61K 31/5377; A61K 31/538; A61K 31/496; C07D 519/00; A61K 31/4545; A61K 31/551; A61N 5/10

(57) ABSTRACT

The present application provides novel optionally substituted fused pyridine and pyrimidine bicyclic compounds and pharmaceutically acceptable salts thereof. Also provided are methods for preparing these compounds. These compounds are useful in co-regulating FAK and/or Src activity by administering a therapeutically effective amount of one or more of the compounds to a subject. By doing so, these compounds are effective in treating conditions associated with the dysregulation of the FAK and/or Src pathway. Advantageously, these compounds perform as dual FAK and/or Src inhibitors. A variety of conditions can be treated using these compounds and include diseases which are characterized by inflammation or abnormal cellular proliferation. In one embodiment, the disease is cancer.

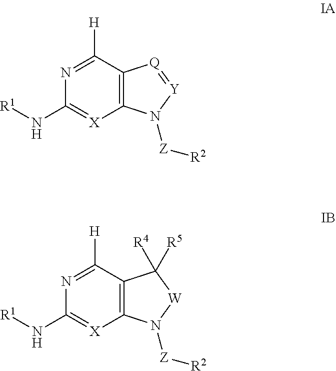

### CROSS-REFERENCE TO RELATED APPLICATIONS

The present application claims the benefit of the filing date of U.S. Provisional Application No. 61/875,963, filed Sep. 10, 2013, entitled Compounds for Regulating FAK and/or SRC Pathways, the disclosure of which is hereby incorporated herein by reference.

### BACKGROUND

Focal adhesion kinase (FAK, also known as FAK1, PTK2) belongs to a family of non-receptor protein tyrosine kinases which transduces signals from integrin and growth factor receptors regulating cell proliferation, migration and survival. FAK derives its name from being localized to cellular focal adhesions or cellular contacts with the extracellular matrix. It is activated by a variety of cell surface receptors and transmits signals to a range of targets.

Cellular interactions with Extracellular Matrix (ECM) serve important roles in normal physiology and pathophysiology including cancer. One of the major mediators of cell adhesion to ECM is Integrin family of receptors. Integrins link ECM to actin cytoskeleton at structures called focal adhesions, thus providing structural rigidity to the cells. In addition, Integrins and their ligands (such as fibronectin, vitronectin, collagen, laminin) also play an important signaling role in the regulation of cellular functions including attachment, spreading, migration, proliferation, and survival. FAK (in partnership with Src) appears to be an obligatory mediator of signaling by most integrins and their ligands in activating intracellular pathways including PI3K/Akt survival pathway.

FAK is a key regulator of survival, proliferation, migration and invasion: processes that are all involved in the development and progression of cancer. FAK overexpression in late-stage cancers is hypothesized to be driven by aberrant growth factor signaling, genetic alterations/mutations, and changes in the microenvironment. The well-known tumor suppressor p53 and the transcription factor nuclear factor κB (NF-κB), have been implicated in regulating FAK gene expression. The overactive growth factor and intracellular signalling is thought to be augmented by FAK and contribute to cell proliferation, cell survival, and cell migration leading to cancer progression and metastasis.

Activated FAK is known to transduce intracellular signals through ERK, PI3K/AKT and JNK pathways affecting transcription. It is shown to induce apoptosis upon detachment from Extracellular Matrix (ECM), proliferation and migration upon growth factor signaling. Based on its role in cellular processes that are critical for development and progression of cancer, FAK has been an attractive therapeutic target.

Several ATP competitive small molecular inhibitors have been developed some of which are currently being tested in the clinic. Collectively, these inhibitors have been shown to inhibit the target in cells, affect cellular processes regulated by FAK, inhibits tumor angiogenesis and tumor growth in multiple tumor models.

PF-573,228 is a selective small molecule FAK inhibitor has been reported to inhibit purified FAK with an IC₅₀ of 4 nM. In A431 cells over-expressing FAK, PF-573,228 inhibited FAK phosphorylation on Tyr397 with an IC₅₀ of 30-100 nM. In addition, treatment with this compound also inhibited both chemotactic and haptotactic migration concomitant with the inhibition of focal adhesion turnover and minimal to no effect on cell proliferation and apoptosis.

Another FAK inhibitor PND-1186 blocks FAK Tyr-397 phosphorylation in vivo and exhibits anti-tumor efficacy in orthotopic breast carcinoma mouse tumor models. PND-1186 (100 mg/kg intraperitoneal, i.p.) showed promising pharmacokinetics (PK) and inhibited tumor FAK Tyr-397 phosphorylation for 12 h. Further, PND-1186 significantly inhibited syngeneic murine 4T1 orthotopic breast carcinoma tumor growth and spontaneous metastasis to lungs.

PHSCN, a synthetic pentapeptide blocks α5β1 integrin-mediated DU145 invasion in vitro and inhibits prostate cancer growth, metastasis, and recurrence in animal models of the disease. Mechanistic studies revealed that PHSCN abolished PHSRN-induced FAK and AKT phosphorylation, as well as PI3K activity in DU145 cells suggesting that anti-cancer activity of PHSCN could at least be partially through inhibition of FAK pathway. Repertaxin, a small-molecule CXCR1 inhibitor, selectively depleted the CSC population in 2 human breast cancer cell lines in vitro and in xenografts through inhibition of FAK/Akt/FOXO3A pathway.

FAK inhibitors could potentially be combined with cytotoxics or targeted therapies to increase efficacy or to overcome resistance. There is evidence of FAK involvement in intrinsic resistance to gemcitabine in pancreatic cancer cell lines. There are several studies demonstrating synergy in anti-tumor activity between FAK inhibition and various cytotoxic drugs including 5-FU, taxanes, platinum, anthracyclines etc.

Pyk2 is closely related to FAK with 60% identity in the kinase domain and 40% identity in the rest of the protein. In addition to structural similarities, Pyk2 shares some functional similarities with FAK as well. FAK has been shown to promote migration and invasion of glioma cells and mediate angiogenesis of pulmonary vascular endothelial cells. More interestingly, in a conditional knock-out mouse model of FAK, Pyk2 was demonstrated to compensate the loss of FAK in restoring the ability of endothelial cells to form vascular networks. Knock-down of both FAK and Pyk2 by shRNA or pharmacological inhibition resulted in complete loss of vessel formation. However, Pyk2 does not seem to compensate all functions of FAK; Fibronectin-stimulated signaling events could be compensated by Pyk2 but not cell migration mediated by FAK.

NVP-TAE226 is a small molecule dual inhibitor of FAK & Pyk2 from Novartis. This compound inhibits FAK with low nanomolar IC₅₀ values in a purified kinase enzymatic assay and oral administration of NVP-TAE226 inhibited 4T1 murine breast tumor growth and metastasis to the lung in a dose-dependent manner. Inhibition of FAK autophosphorylation at Tyrosine397 and Akt phosphorylation at Serine473 was observed in a dose-dependent manner in 4T1 breast carcinoma. NVP-TAE226 also showed strong anti-proliferative effect against a panel of pancreatic cancer cell lines with an IC₅₀ of 0.76 μmol/L. Oral administration of NVP-TAE226 efficiently inhibited MIA PaCa-2 human pancreatic tumor growth efficiently with no body weight loss. PF-562,271 another potent, inhibitor of FAK and Pyk2 (IC₅₀ of 1.5 and 14 nM, respectively) has shown strong inhibition of FAK autophosphorylation in cell culture (EC₅₀, 5 nM) and in vivo (ED₅₀, 93 ng/mL). This compound has also shown good antitumor efficacy and tumor regression in prostate and pancreatic cancer xenograft models with no observed toxicity.

Thus, it may be beneficial to inhibit FAK and Pyk2 simultaneously to overcome the compensatory effect of Pyk2 in angiogenesis and integrin mediated signaling that affect cell survival and proliferation.

Linked activities of focal adhesion kinase (FAK) and steroid receptor coactivator (Src), is a common intracellular point of convergence in the signaling initiated by integrin-ECM interaction. The FAK/Src complex transduces signals from Receptor Tyrosine Kinases (RTKs) and integrates signals from both integrins and RTKs in normal and tumor cells.

The increased expression or activity of FAK and/or Src in tumors is associated with a more invasive and aggressive phenotype and has led to the discovery of Src and FAK inhibitors as new anticancer agents. As individual agents, these inhibitors are able to block proliferation, survival, angiogenesis and/or migration and/or invasion in preclinical tumor models. Several studies have demonstrated that colon cancer cell lines have survival signals operative through both FAK and Src activities, suggesting that the combination of these signals may contribute to their resistance to apoptosis. Furthermore, these results have shown for the first time that combined dual Src and FAK inhibition may be effective for inducing apoptosis in colon cancer cell lines. Some previous studies looking at the role of FAK in breast cancer cells have suggested that up-regulation of FAK in these cells has a dual function: (a) promoting adhesive properties of the tumor cells; and (b) promoting their survival. These results are consistent with a similar role for FAK in colon cancer, whereby inhibition of FAK alone did affect detachment and apoptosis. Furthermore, while the addition of Src inhibition enhanced the rate of detachment of the cells, it had a greater effect on enhancing the rate of apoptosis, operating through AKT-dependent pathways with activation of caspase-3. Thus, these results demonstrate a cooperative role for FAK and Src in suppressing apoptosis in colon cancer cells and support a model whereby FAK and Src together provide survival signals that can be disrupted by dual inhibition of these kinases.

Papillary thyroid cancer samples show high phosphor-Y861-FAK levels and high levels of phopho-Y861-FAK have also been correlated with sensitivity to the Src inhibitor AZD0530 in papillary and also in anaplastic thyroid cancer models. Although the relationship between Src and cancer progression is best documented in colon and breast cancer, Src overexpression or overactivation has also been shown in a variety of primary human tumors and their metastases. Preclinical data from prostate cancer cell lines have also suggested a role for FAK signaling in the induction of VEGF expression in tumor cells. Src has also been associated with VEGF production in tumor cells and Src inhibition decreases angiogenesis in vivo.

Epithelial mesenchymal transition (EMT) is a complex cellular and molecular process by which epithelial cells acquire mesenchymal and migratory properties. Increasing evidence suggests that FAK and Src, through their ability to integrate signals from numerous signaling receptors, play a critical role in tumor-associated EMTs promoting intracellular signaling pathways that lead to the induction of E-cadherin repressors and to the subsequent E-cadherin down-regulation as well as that promote FA turnover to allow tumor cell migration/invasion.

Currently there is strong evidence to suggest inhibition of FAK and Src as a novel and promising anticancer strategy. A synergistic effect has already been shown when they are combined with other anti-cancer agents. There are several on-going trials with FAK and Src inhibitors under clinical development as a single agent or in combination with other therapeutic approaches, which have already shown clinical benefits in cancer subjects with solid tumors.

### BRIEF DESCRIPTION OF THE DRAWINGS

FIG. 1 provides dilutions using the TECAN liquid handler as described in Example 659.

### SUMMARY OF THE INVENTION

In one aspect, a compound of formula IA or IB, or a pharmaceutically acceptable salt or prodrug thereof, is provided and has the following structure, wherein R¹-R⁵, Q, W, X, Y, and Z are defined herein.

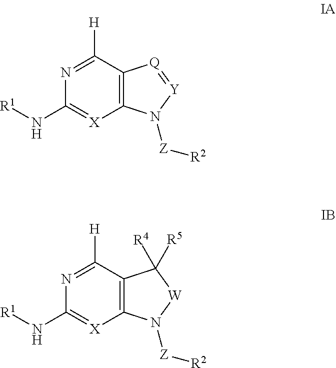

In yet another aspect, a compound of formula IA-2 or IB-2 is provided and has the following structure, wherein R¹-R⁵, W, X, Y, and Z are defined herein.

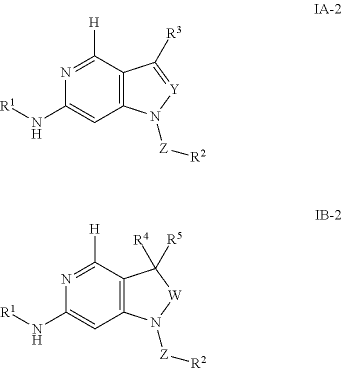

In still a further aspect, a compound of formula IA-3 or IB-3 is provided and has the following structure, wherein R¹-R⁵, W, X, Y, and Z are defined herein.

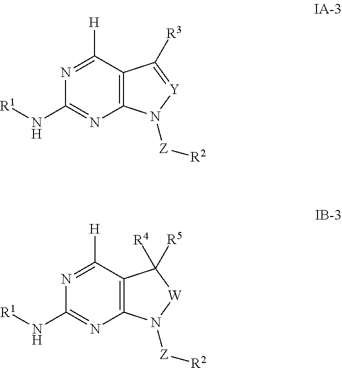

In another aspect, a compound of formula IA-4 or IB-4 is provided and has the following structure, wherein R¹-R⁵, W, X, Y, and Z are defined herein.

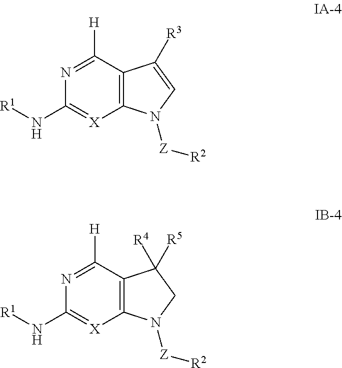

In yet a further aspect, a compound of formula IA-5 is provided and has the following structure, wherein R¹, R², X, Y, and Z are defined herein.

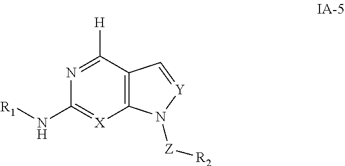

In another aspect, a compound of formula IA-6 is provided and has the following structure, wherein R¹, R², X, Y, and Z are defined herein.

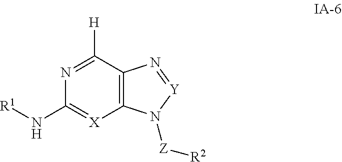

In yet a further aspect, a compound of formula IA-7 is provided and has the following structure, wherein R¹, R², X, and Z are defined herein.

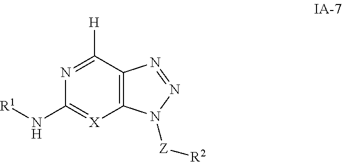

In a further aspect, a compound of formula IA-8 is provided and has the following structure, wherein R¹, R², X, and Z are defined herein.

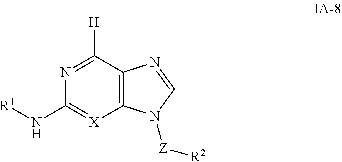

In still another aspect, a pharmaceutical composition is provided and contains one or more compounds described herein and a pharmaceutically acceptable carrier.

In a further aspect, a kit is provided and contains one or more compounds described herein.

In yet another aspect, a method for regulating the FAK pathway, Src pathway, or a combination thereof is provided and comprises administering a therapeutically effective amount of one or more compounds described herein to a subject in need thereof. In one embodiment, the regulation includes inhibiting the FAK and Src pathways.

In a further aspect, a method for treating a condition treatable by inhibiting the FAK pathway, Src pathway, or a combination thereof is provided and comprises administering a therapeutically effective amount of one or more compounds described herein to a subject in need thereof.

In still another aspect, a method of inhibiting the FAK pathway, Src pathway, or a combination thereof is provided and comprises administering one or more compounds described herein to a subject in need thereof. In one embodiment, the method includes comprising inhibiting the FAK and Src pathways.

In yet a further aspect, a method for treating a disease characterized by an abnormal cellular proliferation resulting from a dysregulated FAK pathway, Src pathway, or a combination thereof is provided and comprises administering a therapeutically effective amount of one or more compounds described herein to a subject in need thereof. In one embodiment, the disease is cancer.

In another aspect, a method of treating cancer is provided and comprises administering one or more compounds described herein to a subject in need thereof.

Other aspects and advantages of the invention will be readily apparent from the following detailed description of the invention.

### DETAILED DESCRIPTION OF THE INVENTION

The present invention provides novel compounds which have capabilities in modulating one or both of the FAK and Src pathways. These compounds may be used to treat disease affected by a dysregulation of one or both of the FAK and Src pathways.

In the present invention, the compound is of formula IA or IB, or a pharmaceutically acceptable salt or prodrug thereof.

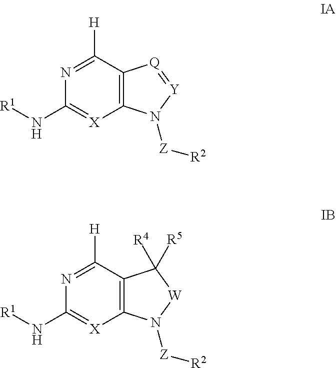

In this structure, X and Y are, independently, N or CH. W is CH₂, —C═O, or NR¹³ and R¹³ is H or optionally substituted alkyl. Z is absent, CH₂, —C═O or —SO₂, with the proviso that (i) Z is not —C═O or —SO₂ when W is C═O and (ii) Z is not absent, when Y is CH. Q is N or CR³.

R¹ is optionally substituted C₆-C₁₀ aryl or optionally substituted C₂-C₁₀ heteroaryl. In one embodiment, R¹ is optionally substituted C₆-C₁₀ aryl. In one example, R¹ is optionally substituted phenyl. In another example, R¹ is phenyl substituted with one or more C₁-C₆ alkoxy, C₁-C₆ alkyl, O—C₃-C₈ cycloalkyl, cycloalkyl, -halogen, C₁-C₆ hydroxyalkyl, CN, NHC(O)C₁-C₆ alkyl, NHC(O)C(C₁-C₆ hydroxalkyl)(C₁-C₆ alkyl), OC₁-C₆ alkyl-N(C₁-C₆ alkyl)₂, C₁-C₆ alkylC(O)NH₂-heterocyclyl, C(O)NH₂, C(O)NH(C₁-C₆ alkyl), C₁-C₆ trifluoroalkyl, C(O)NH(C₁-C₆ alkyl)-O—C₁-C₆ alkyl, N(C₁-C₆ alkyl)₂, NHC(O)C₁-C₆ hydroxyalkyl, NHC(O)(C₃-C₈ cycloalkyl), S(C₁-C₆ alkyl), SO₂NH₂, SO₂(C₁-C₆ alkyl), SO₂—C₁-C₆ trifluoroalkoxy, O—C₁-C₆ alkyl(heterocyclyl), —CH₂—(C₁-C₆ hydroxyalkyl-heterocyclyl), C₁-C₆ alkyl-CN, NH(C₁-C₆ hydroxyalkyl), —(C₁-C₆ alkyl)N(C₁-C₆ alkyl)₂, C(O)(heterocyclyl), oxo-(heterocyclyl), (heterocyclyl), dioxo-(heterocyclyl), N(C₁-C₆ alkyl)₂(heterocyclyl), OC(O)C₁-C₆ alkyl(heterocyclyl), C₁-C₆ hydroxyalkyl(heterocyclyl), OH substituted heterocyclyl, NH(C₁-C₆ alkyl)-(heterocyclyl), C₁-C₆ alkyl-NH₂-heterocyclyl, di-(C₁-C₆ alkyl)N—(C₁-C₆ alkyl)(heterocyclyl), heterocyclyl substituted heterocyclyl, heteroaryl substituted heterocyclyl, NH₂ substituted heterocyclyl, halogen substituted heterocyclyl, —O— (heterocyclyl), NH(C₁-C₆ alkyl)SO₂ (C₁-C₆ alkyl)-phenyl, di-(C₁-C₆ alkyl)(heterocyclyl), C₁-C₆ alkyl(heterocyclyl), C(O)(C₃-C₈ cycloalkyl)heterocyclyl, C(O)(C₁-C₆ alkyl)(heterocyclyl), 1-C₁-C₆ alkyl-O—C(O)C₁-C₆ alkyl-(heterocyclyl), C₁-C₆ alkoxy(heterocyclyl), 1-C₁-C₆ hydroxyalkyl(heterocyclyl), C₃-C₈ cycloalkyl-heterocyclyl, heteroaryl, or oxo-heteroaryl. In a further example, R¹ is phenyl optionally substituted with C₁-C₆ alkoxy, C₁-C₆ alkyl, O—C₃-C₈ cycloalkyl, halogen, C₁-C₆ hydroxyalkyl, CN, NHC(O)C₁-C₆ alkyl, NHC(O)C(C₁-C₆ hydroxalkyl)(C₁-C₆ alkyl), OC₁-C₆ alkyl-N(C₁-C₆ alkyl)₂, C(O)NH₂, C(O)NH(C₁-C₆ alkyl), di-(C₁-C₆ alkyl)N-cyclohexyl, C₁-C₆ trifluoroalkyl, C(O)NH—C1-C₆ alkyl-O—C₁-C₆ alkyl, N(C₁-C₆ alkyl)₂, NHC(O)C₁-C₆ hydroxyalkyl, NHC(O)(C₃-C₈ cycloalkyl), S(C₁-C₆ alkyl), SO₂NH₂, SO₂(C₁-C₆ alkyl), SO₂—C₁-C₆ trifluoroalkoxy, O—C₁-C₆ alkyl-morpholine, O—C₁-C₆ alkyl-piperazine, —CH₂—(C₁-C₆ hydroxyalkyl-pyrrolidine), —C₁-C₆ alkyl-pyrrolidine, C₁-C₆ alkyl-CN, NH(C₁-C₆ hydroxyalkyl), NH(C₁-C₆ alkyl), —(C₁-C₆ alkyl)N(C₁-C₆ alkyl)₂, C(O)morpholine, oxo-morpholine, morpholine, dioxo-thiomorpholine, thiomorpholine, N(C₁-C₆ alkyl)₂-piperidine, OC(O)C₁-C₆ alkyl-piperidine, C₁-C₆ hydroxyalkyl-piperidine, 4-OH-piperidine, NH(C₁-C₆ alkyl)-piperidine, di-(C₁-C₆ alkyl)N—(C₁-C₆ alkyl)-piperidine, C₁-C₆ alkyl-NH₂-piperidine, morpholine-piperidine, piperazine-piperidine, OH-piperidine, NH₂-piperidine, C₁-C₆ alkoxy-piperidine, piperidine, tetrazole, N(C₁-C₆ alkyl)₂-pyrrolidine, N(C₁-C₆ alkyl)₂-azetidine, azetidin-3-yloxy, NH(C₁-C₆ alkyl)SO₂ (C₁-C₆ alkyl)-phenyl, pyridine, oxo-pyridine, tetrahydropyridine, piperazine, (C₁-C₆ alkyl)₂piperazine, C₁-C₆ alkyl-piperazine, C(O) (C₃-C₈ cycloalkyl)-piperazine, C(O)(C₁-C₆ alkyl)-piperazine, 1-C₁-C₆ alkyl-O—C(O)C₁-C₆ alkyl-piperazine, C₁-C₆ alkoxy-piperazine, 1-C₁-C₆ hydroxyalkyl-piperazine, oxo-piperazine, C₃-C₈ cycloalkyl-piperazine, C(O)-piperazine, C₁-C₆ alkyl-diazepane, diazepane, 2,5-diaza-bicyclo[2.2.1]hept-2-yl optionally substituted with C₁-C₆ alkyl and C(O)(C₁-C₆ alkyl), hexahydro-pyrrolo[1,2-a]pyrazine optionally containing an oxo group in the backbone of the ring. In yet another example, R¹ is 1-OCH₃-3-CH₃-phenyl, 2-(O-cyclobutyl)-4-piperazin-4-yl-phenyl, 2,6-di-F-phenyl, 4-(N-methylaminocarbonyl)-phenyl, 4-(4-(CH₃)₂N-cyclohexyl)-phenyl, 2-CH₂OH-4-piperazin-4-yl-phenyl, 2-CH₃-4-(1-CH₂CH₂OH-piperazin-4-yl)-phenyl, 2-CH₃-phenyl, 2-CH₃-piperazin-4-yl-phenyl, 2-CN-phenyl, 2-F-4-(2-CH₃-piperazin-4-yl)-phenyl, 2-F-4-piperazin-4-yl-phenyl, 2-F-5-NHC(O)CH₃-phenyl, 1-CH₂C(O)NH₂-piperidin-4-yl-phenyl, 4,4-di-F-piperidin-1-yl-phenyl,-2-F-phenyl, 2-NHC(O)CH₃-phenyl, 2-OCH₃-3-F-4-C(O)morpholine-phenyl, 2-OCH₃-3-F-4-piperazin-4-yl-phenyl, 2-OCH₃-4-(1-CH₂CH₂OH-piperazin-4-yl)-phenyl, 2-OCH₃-4-(1-CH₃-piperazin-4-yl)-phenyl, 2-OCH₃-4-(4-N(CH₃)₂-piperidin-1-yl)-phenyl, 2-OCH₃-4-(4-OC(O)CH₃-piperidin-1-yl)-phenyl, 2-OCH₃-4-(piperidin-4-ol)-phenyl, 3-CH₃-4-(1-(2-dimethylaminoethyl)-piperidin-4-yl)-phenyl, 2-OCH₃-4-C(O)-morpholine-phenyl, 2-OCH₃-4-morpholine-phenyl, 2-OCH₃-4-piperazin-4-yl-phenyl, 2-OCH₃-phenyl, 3-NHC(O)C(CH₃)₂OH-phenyl, 3-(tetrazol-5-yl)-phenyl, 3-OCH₂CH₂N(CH₃)₂)-phenyl, 3,4,5-tri-OCH₃-phenyl, 3-C(O)NH₂-phenyl, 3-C(O)NHCH₃-phenyl, 3-CF₃-phenyl, 3-CH₂OCH₃-phenyl, 3-CH₂OH-4-(2-CH₃-piperazinyl)-phenyl, 3-CH₂OH-4-(piperazin-4-yl)-phenyl, 3-CH₃-(1-CH₃-piperazin-4-yl)-phenyl, 3-CH₃-4-(3-CH₃-piperazinyl)-phenyl, 3-CH₃-4-(4-N(CH₃)₂-piperidin-1-yl)-phenyl, 3-CH₃-4-morpholine-phenyl, 3-CH₃-4-piperazin-4-yl-phenyl, 3-CN-phenyl, 3-F-4-(2-CH₃-piperazinyl)-phenyl, 3-F-4-(3-CH₃-piperazinyl)-phenyl, 3-F-4-(3-N(CH₃)₂-pyrrolidin-1-yl)-phenyl, 3-F-4-(4-N(CH₃)₂-piperidin-1-yl)-phenyl, 3-F-4-(C(O)NHCH₂C(CH₃)₂OCH₃)-phenyl, 3-F-4-(C(O)NHCH₂C(CH₃)OH)-phenyl, 3-F-4-(N(CH₃)₂-azetidin-1-yl)-phenyl, 3-F-4-C(O)-morpholine-phenyl, 3-F-4-piperazin-4-yl-phenyl, 3-F-phenyl, 3-morpholine-phenyl, 3-N(CH₃)₂-phenyl, 3-NHC(O)CH(CH₃)OH-phenyl, 3-NHC(O)CH₃-phenyl, 3-OCH₃-4-(1-CH₂CH₂OH-piperazin-4-yl)-phenyl, 3-OCH₃-4-(3-CH₃-piperazin-4-yl)-phenyl, 3-OCH₃-4-(4-piperidin-4-ol)-phenyl, 3-OCH₃-4-morpholine-phenyl, 3-OCH₃-4-NHC(O)(cyclobutyl)-phenyl, 3-OCH₃-4-NHC(O)(cyclopropyl)-phenyl, 3-OCH₃-4-piperazin-4-yl-phenyl, 3-OCH₃-phenyl, 4-(3-OH-piperidin-1-yl)-phenyl, 3-piperazine-phenyl, 3-piperazinyl-5-(3-N(CH₃)SO₂CH₃-phenyl)-phenyl, 3-piperazinyl-5-(pyridin-3-yl)-phenyl, 3-SCH₃-phenyl, 3-SO₂NH₂-phenyl, 4-(1,1-dioxo-thiomorpholin-4-yl)-phenyl, 4-(1,2-di-CH₃-piperazin-4-yl)-phenyl, 4-(1-C(O)(cyclopropyl))piperazin-1-yl-phenyl, 4-(1-C(O)CH₃)-piperazin-4-yl-phenyl, 4-(1-C(O)CH₃-2-CH₃-piperazin-4-yl)-phenyl, 4-(1-CH₂CH₂O—C(O)CH₃-piperazin-4-yl)-phenyl, 4-(1-CH₂CH₂OCH₃-piperazin-4-yl)-phenyl, 4-(1-CH₂CH₂OH-piperazin-4-yl)-phenyl, 4-(1-CH₂CH₃-piperazin-4-yl)-phenyl, 4-(1-CH₃-piperazin-4-yl)-phenyl, 4-(2,5-di-CH₃-piperazin-4-yl)-phenyl, 4-(2-CH₃-piperazin-4-yl)-phenyl, 4-(2-oxo-morpholine)-phenyl, 4-(2-oxo-piperazinyl-phenyl, 4-(2-oxo-pyridin-1-yl)-phenyl, 4-(3,3-di-CH₃-piperazinyl)-phenyl, 4-(3,5-di-CH₃-piperazin-4-yl)-phenyl, 4-(3-CH₂CH₃-piperazinyl-1-yl)-phenyl, 4-(3-CH₂NH₂-piperidin-1-yl)-phenyl, 4-(3-CH₂OH-piperidin-1-yl)-phenyl, 4-(3-CH₃-piperazin-1-yl)-phenyl, 4-(2-oxo-3,3-di-CH₃-piperazin-1-yl)-phenyl, di-(C₁-C₆ alkyl)-oxo-piperazine, 4-(3-N(CH₃)₂-azetidin-4-yl)-phenyl, 4-(3-N(CH₃)₂-piperidin-1-yl)-phenyl, 4-(4-(azetidin-3-yloxy)-phenyl, 4-(4-(morpholinyl)-piperidin-1-yl)-phenyl, 4-(4-(piperazin-1-yl)-piperidin-1-yl)-phenyl, 4-(4-CH₂CH(CH₃)OH-piperazin-1-yl)-phenyl, 4-(4-CH₂CH(CH₃)-piperazin-1-yl)-phenyl, 4-(4-CH₂OH-piperidin-1-yl)-phenyl, 4-(4-CH₃-4-OH-piperidin-1-yl)-phenyl, 4-(4-CH₃-piperazin-1-yl)-phenyl, 4-(4-cyclopropyl-piperazinyl-1-yl)-phenyl, 4-(4-methyl-[1,4]diazepan-1-yl)-phenyl, 4-(4-N(CH₃)₂-piperidin-1-yl)-phenyl, 4-(4-N(CH₃)CH₂CH₂OH-piperidin-1-yl)-phenyl, 4-(4-NH₂-piperidin-1-yl)-phenyl, 4-(4-NHCH₃-piperidin-1-yl)-phenyl, 4-(4-OC(O)CH₃-piperidin-1-yl)-phenyl, 4-(4-piperidin-1-OCH₃)-phenyl, 4-(NHC(O)C(CH₃)₂OH)-phenyl, 4-(OCH₂CH₂-morpholine)-phenyl, 4-(OCH₂CH₂-piperazin-4-yl)-phenyl, 4-(O-piperidin-4-yl)-phenyl, 4-(piperazin-2-yl)-phenyl, 4-(piperazine-2-one)-phenyl, 4-(piperazinyl-1-yl)-phenyl, 4-(piperidin-4-ol)-phenyl, 4-[1,4]diazepan-1-yl-phenyl, 4-C(O)-piperazin-4-yl-phenyl, 4-CH₂-(2-CH₂OH)-pyrrolidin-1-yl-phenyl, 4-CH₂-piperazin-4-yl-phenyl, 4-CH₂-pyrrolidin-1-yl-phenyl, 4-CH₂CN-phenyl, 4-CN-phenyl, 4-F-phenyl, 4-morpholine-phenyl, 4-N(CH₃)₂-phenyl, 4-NHCH₂CH₂OH-phenyl, 4-NHC(O)(cyclopropyl)-phenyl, 4-NHC(O)CH(CH₃)₂-phenyl, 4-NHC(O)CH₃-phenyl, 4-NHCH₃-phenyl, 4-O-(4-NHCH₃-piperidin-1-yl)-phenyl, 4-OCH₃-phenyl, 4-piperazin-4-yl-phenyl, 4-piperidin-4-yl-phenyl, 4-SO₂CH₃-phenyl, 4-SO₂—CHF₂-phenyl, 4-tetrahydropyridin-4-yl-phenyl, 4-thiomorpholino-phenyl, CH₂N(CH₃)₂-phenyl, phenyl, NHC(O)CH₃-phenyl, OCH₂CH₂N(CH₃)₂-phenyl, 3-OCH₃-4-(5-methyl-(2,5-diaza-bicyclo[2.2.1]hept-2-yl))-phenyl, 4-(2,5-diaza-2-C(O)CH₃-bicyclo[2.2.1]hept-2-yl)-phenyl, 4-(2,5-diaza-bicyclo[2.2.1]hept-2-yl)-phenyl, 4-(6-oxohexahydropyrrolo[1,2-a]pyrazin-2-yl)-phenyl, 4-(hexahydro-pyrrolo[1,2-a]pyrazin-2-yl)-phenyl.

In another embodiment, R¹ is optionally substituted C₂-C₁₀ heteroaryl. In one example, R¹ is optionally substituted pyrazole, quinoline, pyridine, pyrimidine, dihydrobenzooxazole, benzooxazole, benzoimidazole, dihydroisobenzofuran, isobenzofuran, dihydrobenzooxazine, benzooxazine, benzotriazole, benzothiazole, benzothiophene, indazole, hexahydropyrazinoindole, indoline, or tetrahydroquinolinyl. In another example, R¹ contains an oxo group in the backbone of said heteroaryl. In a further example, R¹ is substituted with one or more C₁-C₆ alkyl, optionally substituted heterocyclyl, CN, NHC(O)(C₁-C₆ alkyl), optionally substituted heteroaryl, —(C₁-C₆ alkyl)N(C₁-C₆ alkyl)₂, or C₁-C₆ hydroxyalkyl. In still another example, R¹ is substituted with one or more C₁-C₆ alkyl, piperidine, CN, NHC(O)(C₁-C₆ alkyl), piperazin-2-one, morpholine, C₁-C₆ alkyl substituted pyridine, —(C₁-C₆ alkyl)N(C₁-C₆ alkyl)₂, or C₁-C₆ hydroxyalkyl. In yet a further example, R¹ is 1-(piperidin-4-yl)-pyrazol-4-yl, 1-CH₃-pyrazol-4-yl, 1-methyl-2-oxo-1,2,3,4-tetrahydroquinolin-6-yl, 2-dihydroquinoline-2-one, 2-oxo-1,2,3,4-tetrahydro-quinolin-6-yl, 6-quinolinyl, 2-CN-pyridin-5-yl, 2-NHC(O)CH₃-pyridin-6-yl, 3-(piperazine-2-one)-pyridin-6-yl, 3-morpholine-pyridin-6-yl, 3-NHC(O)CH₃-pyridin-6-yl, 3-pyridinyl, 4-(3-CH₃-piperazinyl)-pyridin-3-yl, 4-pyridinyl, pyrimidinyl, 2,3-dihydro-benzooxazol-2-one, 5-benzoimidazolyl, 5-benzimidazol-2-one, 2-oxo-2,3-dihydro-benzoimidazol-5-yl, 3-oxo-1,3-dihydroisobenzofuran-5-yl, 3-oxo-3,4-dihydro-benzooxazin-7-yl, 3-oxo-3,4-dihydro-benzoxazin-6-yl, 3,4-dihydro-benzooxazin-6-yl, 1,4-benzoxazin-7-yl, 5-benzotriazolyl, 6-benzothiazolyl, 6-benzothiophene-yl, 6-indazolyl, 1,2,3,4,10,10a-hexahydro-pyrazino[1,2-a]indol-8-yl, 1,3-dioxo-2,3-dihydro-isoindol-5-yl, 1-CH₂CH₂N(CH₃)₂-2-oxo-indolin-5-yl, 1-CH₂CH₂OH-2-oxo-indol-5-yl, 1-methyl-2-oxo-2-3-dihydro-indol-5-yl, 3-(1,2,3,6-tetrahydropyridin-4-yl)-indol-5-yl, 3-(tetrahydropyridin-4-yl)-indol-5-yl, 5-indolin-2-one, or 5-indolinyl. In another example, R¹ is heteroaryl substituted with one or more R⁶. R⁶ is H, optionally substituted C₁-C₆ alkyl, alkoxy, —O(CH₂)ₐNR⁸R⁹, —O(CH₂)ₐOH, —O(CH₂)ₐO—C₁-C₆ alkyl, CN, optionally substituted aryl, optionally substituted heteroaryl, monocyclic or bicyclic optionally substituted cycloalkyl, monocyclic or bicyclic optionally substituted heterocyclyl, monocyclic or —O-bicyclic optionally substituted heterocyclyl, (aryl)alkyl, COOH, NH₂, NR⁸R⁹, —CONH₂, —CONR¹⁰R¹¹, —S-(optionally substituted C₁-C₆ alkyl), —SO₂—C₁-C₆ alkyl, —SO₂—NH—C₁-C₆ alkyl, —SO₂—N(C₁-C₆ alkyl)₂, —NHCOC₁-C₆-alkyl, —NHCOC₁-C₆-alkyl substituted with OH, —SO₂NH₂, —SO₂NR¹⁰R¹¹, aminoalkyl, (alkyl)amido, (alkyl)amino, arylalkyl, alkylcarboxyl, (alkyl)carboxyamido, heterocyclyl(alkyl), heteroaryl(alkyl), (aryl)oxy, (heteroaryl)oxy, halogen, hydroxyalkyl, —S(O)ₙ-perfluoroalkyl, perfluoroalkyl, monofluoroalkyloxy, difluoroalkyloxy or perfluoroalkyloxy. In one embodiment, R⁶ is an aminoalkyl. In another embodiment, R⁶ is —(CH₂)ₐNH₂ or —(CH₂)ₐNR⁸R⁹. In a further embodiment, R⁶ is an alkylcarboxyl. In still another embodiment, R⁶ is —OC(O)C₁-C₆ alkyl. a is 2-6 and n is 0-2. R⁸ and R⁹ are, independently, H, optionally substituted C₁-C₆ alkyl, —COC₁-C₆ alkyl, —COC₃-C₆ cycloalkyl, —CO-heterocyclyl, —CONR¹⁰R¹¹, —SO₂-optionally substituted C₁-C₆ alkyl, —SO₂ (alkyl) (C₁-C₆ hydroxyalkyl), —SO₂ (alkyl)(C₁-C₆ alkoxyalkyl), —SO₂ (alkyl)(C₁-C₆ alkylamino), —SO₂-aryl, —SO₂-heteroaryl, —SO₂—C₃-C₇ cycloalkyl, —SO₂—C₄-C₆ heterocyclyl, —COO—C₁-C₆ alkyl, —COO—C₃-C₆ cycloalkyl, —COO—C₄-C₆ heterocyclyl, C₁-C₆ alkylcarbonyl, C₁-C₆ hydroxyalkyl. Alternatively, R⁸ and R⁹ are taken together to form a 3 to 7 membered saturated or partially saturated ring optionally having 1 or more heteroatom and wherein said 3 to 7 membered ring is optionally substituted with optionally substituted C₁-C₆ alkyl, hydroxyl, halogen, C₁-C₆ alkoxy, NH₂, or NR⁸R⁹, or one or more —CH₂— of the ring are replaced by C═O. R¹⁰ and R¹¹ are, independently, H, C₁-C₆ alkyl, aryl, heteroaryl, mono cycloalkyl, bicyclic cycloalkyl, mono heterocyclyl, or bicyclic heterocyclyl. Alternatively, R¹⁰ and R¹¹ are taken together to form a 3 to 7 membered saturated or partially saturated ring optionally having 1 or more heteroatom and wherein said 3 to 7 membered ring is optionally substituted with C₁-C₆ alkyl or one or more —CH₂— of the ring are replaced by C═O, (aryl)alkyl, hydroxyalkyl or perfluoroalkyl.

R³ is H, C₁-C₆ alkyl, halogen, CN, or C₁-C₆ trifluoroalkyl. In one embodiment, R³ is H, CH₃, or F.

R⁴ and R⁵ are, independently, H, F or optionally substituted C₁-C₆ alkyl. In one embodiment, R⁴ and R⁵ are, independently, H or CH₃. Alternatively, R⁴ and R⁵ are taken together to form a 3 to 6 membered cyclic ring having 0-1 heteroatom. In one embodiment, R⁴ and R⁵ are taken together to form a 3-8 membered cyclic system having it its backbone one O, S(O)ₙ or NR⁷, wherein R⁷ is H, optionally substituted C₁-C₆ alkyl, —SO₂-(optionally substituted C₁-C₆ alkyl, (alkoxy)carbonyl-, (alkyl)amido-, —CO—C₁-C₆ alkyl or alkoxy alkyl. In another embodiment, R⁴ and R⁵ are joined to form cyclopropyl.

R² is optionally substituted C₆-C₁₀ aryl, optionally substituted heteroaryl, C₃-C₆ optionally substituted cycloalkyl or C₂-C₆ heterocyclyl. In one embodiment, R² is optionally substituted heteroaryl. In one example, R² is optionally substituted imidazole, pyridine, thiophene, quinoline, naphthalene, benzothiazole, or benzothiodiazole. In another example, R² is optionally substituted imidazole. In a further example, R² is imidazole substituted with 1 or 2 C₁-C₆ alkyl. In yet another example, R² is imidazole substituted with 1 or 2 CH₃ groups. In still a further example, R² is optionally substituted pyridine. In another example, R² is pyridine substituted with 1 or more C₁-C₆ alkoxy, N(C₁-C₆ alkyl)OSO₂(C₁-C₆ alkyl), N(C₁-C₆ alkyl)(SO₂(C₁-C₆ alkyl), or N(C₁-C₆ alkyl)SO₂(C₃-C₈ cycloalkyl). In a further example, R² contains a SO₂ group in the backbone of ring. In still a further example, R² is 1-N(CH₃)(OSO₂CH₃)-pyridin-2-yl, 1-N(CH₃)SO₂CH₃-pyridin-2-yl, 2-N(CH₃)SO₂CH₃-pyridin-3-yl, pyridine-2-yl, pyridine-3-yl, 2-OCH₃-pyridin-4-yl, 2-N(CH₃)SO₂-cyclopropyl-pyridin-3-yl, or dioxidoisothiazolidin-2-yl. In yet another example, R² is optionally substituted quinoline. In a further example, R² is quinoline substituted with 1 or more C₁-C₆ alkyl. In still another example R² is quinoline containing a C(O) in the backbone of the ring. In a further example, R² is quinolone, 4-CH₃-quinolin-8-yl, 2-CH₃-quinolin-8-yl, 6-CH₃-quinolin-8-yl, or 8-isoquinoline. In yet another example, R² is thiophene.

In another embodiment, R² is optionally substituted aryl. In one example, R² is optionally substituted phenyl. In another example, R² is phenyl substituted with 1 or more of halogen, C₁-C₆ alkoxy, C₁-C₆ trifluoroalkyl, C₁-C₆ alkyl, CN, NH₂, C₁-C₆ trifluoroalkoxy, SO₂N(C₁-C₆ alkyl)₂, SO₂NH(C₁-C₆ alkyl), SO₂(C₁-C₆ alkyl), N(C₁-C₆ alkyl)SO₂ (C₁-C₆ alkyl), N(C₃-C₈ cycloalkyl)SO₂ (C₁-C₆ alkyl), NHC(O)(C₁-C₆ alkyl), N(C₁-C₆ hydroxyalkyl)SO₂(C₁-C₆ alkyl), N(alkylamino)SO₂(C₁-C₆ alkyl), N(C₁-C₆ alkoxy)SO₂(C₁-C₆ alkyl), —(C₁-C₆ alkyl)-C(O)NH(C₁-C₆ alkyl), or N(C₁-C₆ alkyl-morpholine)SO₂(C₁-C₆ alkyl). In a further example, R² is phenyl, 2,3-di-Cl-phenyl, 2,5-di-Cl-phenyl, 2,5-di-OCH₃-phenyl, 2-5-di-Cl-phenyl, 2-CF₃-phenyl, 2-CH₃-phenyl, 2-Cl-5-CH₃-phenyl, 3-Cl-phenyl, 3-CN-phenyl, 2-Cl-phenyl, 2-F-phenyl, 2-OCF₃-phenyl, 2-OCH₃-phenyl, 2-NH₂-phenyl, 4-tolyl, 3-OCH₃-phenyl, 4-OCF₃-phenyl, 3-OCF₃-phenyl, 2-OCHF₂-phenyl, 2-SO₂N(CH₃)₂-phenyl, 2-NHSO₂CH₃-phenyl, 2-SO₂—NHCH₃-phenyl, 3-SO₂—NHCH₃-phenyl, 3-SO₂(CH₃)-phenyl, 2-N(CH₃)SO₂CH₃-phenyl,2-N(CH₃)SO₂CH₃-3-OCH₃-phenyl, 2-N(cyclopropyl)SO₂CH₃-phenyl, 3-N(CH₃)SO₂ (CH₃)-phenyl, 2-NH(CH₃)—SO₂CH₃-phenyl, 4-NHC(O)CH₃-phenyl, 2-N(CH₂CH₂OH)SO₂CH₃-phenyl, 2-N(CH₂CH₂NH₂)SO₂CH₃-phenyl, 2-N(CH₂CH₂OCH₃)SO₂CH₃-phenyl, 2-CH₂—C(O)NHCH₃-phenyl, 2-N(CH₂CH₂-morpholine)SO₂CH₃-phenyl.

In a further embodiment, R² is optionally substituted C₃-C₈ cycloalkyl. In one example, R² is cyclopentyl or cyclopropyl.

In another embodiment, R² is C₆-C₁₀ aryl or heteroaryl substituted with one or more R¹². R¹² is H, optionally substituted C₁-C₆ alkyl, optionally substituted C₂-C₆ alkenyl, optionally substituted C₂-C₆ alkynyl, alkoxy, —S(O)ₙ—C₁-C₆ alkyl, —O(CH₂)ₐNR⁸R⁹, —O(CH₂)ₐOH, —O(CH₂)ₐO—C₁-C₆ alkyl, CN, aryl, heteroaryl, optionally substituted monocyclic cycloalkyl, optionally substituted bicyclic cycloalkyl, optionally substituted monocyclic heterocyclyl, optionally substituted bicyclic heterocyclyl, (aryl)alkyl, COOH, NH₂, NR⁸R⁹, —CONH₂, —CONR¹⁰R¹¹, —SO₂NH₂, —SO₂NR¹⁰R¹¹, aminoalkyl, (alkyl)amido, (alkyl)amino, arylalkyl, alkylcarboxyl, (alkyl)carboxyamido, heterocyclyl(alkyl), heteroaryl(alkyl) (aryl)oxy, (heteroaryl)oxy, halogen, hydroxyalkyl, perfluoroalkyl, monofluoroalkyloxy, difluoroalkyloxy or perfluoroalkyloxy. a is 2-6 and n is 0-2. In one example, R¹² is an aminoalkyl. In another example, R¹² is —(CH₂)ₐNH₂ or —(CH₂)ₐNR⁸R⁹. In a further example, R¹² is an alkylcarboxyl. In still a further example, R¹² is —OC(O)C₁-C₆ alkyl. R⁸ and R⁹ are, independently, H, optionally substituted C₁-C₆ alkyl, —COC₁-C₆ alkyl, —COC₃-C₆ cycloalkyl, —CO-heterocyclyl, —CONR¹⁰R¹¹, —SO₂— optionally substituted C₁-C₆ alkyl, —SO₂(alkyl) (C₁-C₆ hydroxyalkyl), —SO₂(alkyl)(C₁-C₆ alkoxyalkyl), —SO₂(alkyl)(C₁-C₆ alkylamino), —SO₂-aryl, —SO₂-heteroaryl, —SO₂—C₃-C₇ cycloalkyl, —SO₂—C₄-C₆ heterocyclyl, —COO—C₁-C₆ alkyl, —COO—C₃-C₆ cycloalkyl, —COO—C₄-C₆ heterocyclyl, C₁-C₆ alkylcarbonyl, C₁-C₆ hydroxyalkyl. Alternatively, R⁸ and R⁹ are taken together to form a 3 to 7 membered saturated or partially saturated ring optionally having 1 or more heteroatom and wherein said 3 to 7 membered ring is optionally substituted with C₁-C₆ alkyl, hydroxyl, halogen, C₁-C₆ alkoxy, NH₂, or NR⁸R⁹, or one or more —CH₂— of the ring are replaced by C═O. R¹⁰ and R¹¹ are, independently, H, C₁-C₆ alkyl, aryl, heteroaryl, mono cycloalkyl, bicyclic cycloalkyl, mono heterocyclyl, or bicyclic heterocyclyl. Alternatively, R¹⁰ and R¹¹ are taken together to form a 3 to 7 membered saturated or partially saturated ring optionally having 1 or more heteroatom and wherein said 3 to 7 membered ring is optionally substituted with C₁-C₆ alkyl or one or more —CH₂— of the ring are replaced by C═O, (aryl)alkyl, hydroxyalkyl or perfluoroalkyl.

In one embodiment, the compound is of formula IA, wherein R¹-R³, X, Y, and Z are defined above.

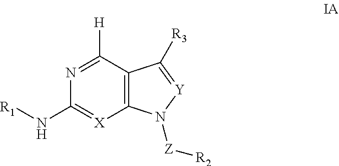

In another embodiment, the compound is of formula IB, wherein R¹, R², R⁴, R⁵, W, X, and Z are defined above.

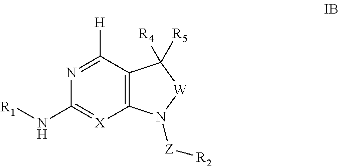

In a further embodiment, the compound is of formula IA-2 or IB-2, wherein R¹-R⁵, W, X, Y, and Z are defined above.

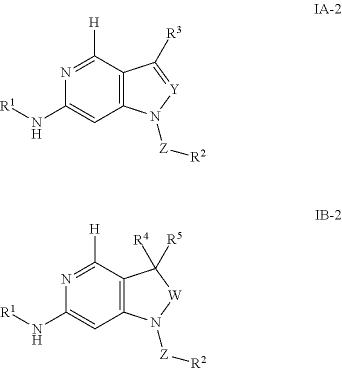

In still another embodiment, the compound is of formula IA-3 or IB-3, wherein R¹-R⁵, W, X, Y, and Z are defined above.

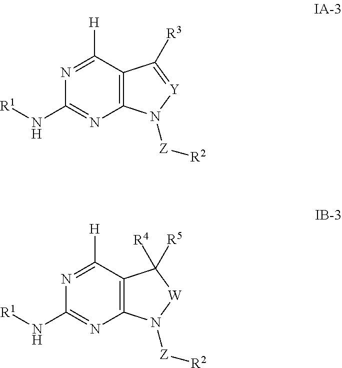

In a further embodiment, the compound is formula IA-4 or IB-4, wherein R¹-R⁵, X and Z are defined above.

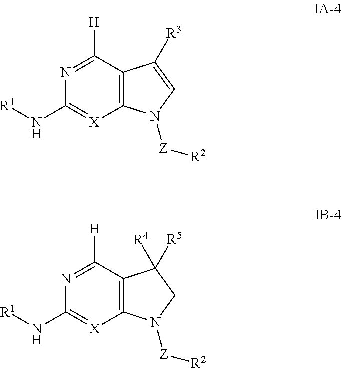

In another embodiment, the compound is of formula IA-5, wherein R¹, R², X, Y, and Z are defined above.

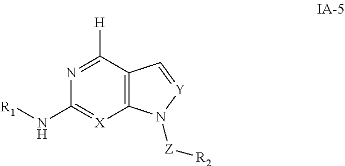

In a still a further embodiment, the compound is of formula IA-6, wherein R¹, R², X, Y, and Z are defined herein.

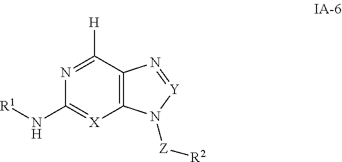

In yet another embodiment, the compound is of formula IA-7, wherein R¹, R², X, and Z are defined herein.

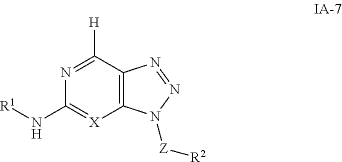

In a further embodiment, the compound is of formula IA-8, wherein R¹, R², X, and Z are defined herein.

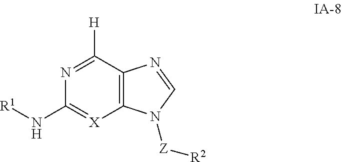

Some compounds within the present invention possess one or more chiral centers, and the present invention includes each separate enantiomer of such compounds as well as mixtures of the enantiomers. Where multiple chiral centers exist in compounds of the present invention, the invention includes each possible combination of chiral centers within a compound, as well as all possible enantiomeric mixtures thereof. All chiral, diastereomeric, and racemic forms of a structure are intended, unless the specific stereochemistry or isomeric form is specifically indicated. It is well known in the art how to prepare optically active forms, such as by resolution of racemic forms or by synthesis from optically active starting materials.

The following definitions are used in connection with the compounds of the present invention unless the context indicates otherwise. In general, the number of carbon atoms present in a given group is designated “Cx-Cy”, where x and y are the lower and upper limits, respectively. For example, a group designated as “C₁-C₆” contains from 1 to 6 carbon atoms. The carbon number as used in the definitions herein refers to carbon backbone and carbon branching, but does not include carbon atoms of the substituents, such as alkoxy substitutions and the like. Unless indicated otherwise, the nomenclature of substituents that are not explicitly defined herein are arrived at by naming from left to right the terminal portion of the functionality followed by the adjacent functionality toward the point of attachment. For example, the substituent “arylalkyloxycarbonyl” refers to the group (C₆-C₁₄ aryl)-(C₁-C₆ alkyl)-O—C(O)—. The term optionally substituted refers to replacing a hydrogen atom of a group with an alkyl, alkoxy, aryl, monocyclic or bicyclic cycloalkyl, mono or bicyclic heterocyclylalkyl, (aryl)alkyl, (alkoxy)carbonyl, (alkyl)amido, (alkyl)amino, —NH₂, aminoalkyl, alkylcarboxyl, (alkyl)carboxyamido, (aryl)amino, haloalkyl, heteroaryl, heterocyclyl, heteroaryl(alkyl), mono, di or perfluoroalkyl, halogen, CN, C(O)OH, amide, amide formed from a primary or secondary amine, NO₂, OH, mono-fluoroalkoxy, di-fluoroalkoxy, trifluoroalkoxy, and hydroxyalkyl. Terms not defined herein have the meaning commonly attributed to them by those skilled in the art.

“Alkyl” refers to a hydrocarbon chain that may be a straight chain or branched chain, containing the indicated number of carbon atoms, for example, a C₁-C₁₂ alkyl group has from 1 to 12 (inclusive) carbon atoms in it. Examples of C₁-C₆ alkyl include, but are not limited to, methyl, ethyl, propyl, butyl, pentyl, hexyl, isopropyl, isobutyl, sec-butyl, tert-butyl, isopentyl, neopentyl, and isohexyl. Examples of C₁-C₈ alkyl include, but are not limited to, methyl, propyl, pentyl, hexyl, heptyl, 3-methylhex-1-yl, 2,3-dimethylpent-2-yl, 3-ethylpent-1-yl, octyl, 2-methylhept-2-yl, 2,3-dimethylhex-1-yl, and 2,3,3-trimethylpent-1-yl. An alkyl group may be unsubstituted or substituted with one or more of halogen, NH₂, (alkyl)NH, (alkyl)(alkyl)N—, —N(alkyl)C(O)(alkyl), —NHC(O)(alkyl), —NHC(O)H, —C(O)NH₂, —C(O)NH(alkyl), —C(O)N(alkyl)(alkyl), CN, OH, alkoxy, alkyl, C(O)OH, —C(O)O(alkyl), —C(O)(alkyl), aryl, heteroaryl, heterocyclyl, cycloalkyl, haloalkyl, aminoalkyl-, —OC(O)(alkyl), carboxyamidoalkyl-, NO₂, and alkylCN.

“Alkenyl” refer to a straight or branched chain unsaturated hydrocarbon containing at least one double bond, and may exist in the E or Z conformation. Examples of C₂-C₈alkenyl include, but are not limited to, ethylene, propylene, 1-butylene, 2-butylene, isobutylene, sec-butylene, 1-pentene, 2-pentene, isopentene, 1-hexene, 2-hexene, 3-hexene, isohexene, 1-heptene, 2-heptene, 3-heptene, 1-octene, 2-octene, 3-octene, and 4-octene. Examples of a C₂-C₆alkenyl group include, but are not limited to, ethylene, propylene, 1-butylene, 2-butylene, isobutylene, sec-butylene, 1-pentene, 2-pentene, isopentene, 1-hexene, 2-hexene, 3-hexene, and isohexene. Examples of C₃-C₈ alkenyl include, but are not limited to, propylene, 1-butylene, 2-butylene, isobutylene, sec-butylene, 1-pentene, 2-pentene, isopentene, 1-hexene, 2-hexene, 3-hexene, isohexene, 1-heptene, 2-heptene, 3-heptene, 1-octene, 2-octene, 3-octene, and 4-octene. Examples of C₃-C₆alkenyl include, but are not limited to, prop-2-enyl, but-3-enyl, but-2-enyl, 2-methyallyl, pent-4-enyl, and hex-5-enyl. An alkenyl group may be unsubstituted or substituted with one or more of halogen, NH₂, (C₁-C₆ alkyl)NH—, (C₁-C₆ alkyl)(C₁-C₆ alkyl)N—, —N(C₁-C₃ alkyl)C(O)(C₁-C₆ alkyl), —NHC(O)(C₁-C₆ alkyl), —NHC(O)H, —C(O)NH₂, —C(O)NH(C₁-C₆ alkyl), —C(O)N(C₁-C₆alkyl)(C₁-C₆ alkyl), CN, OH, C₁-C₆ alkoxy, C₁-C₆ alkyl, —C(O)OH, —C(O)O(C₁-C₆ alkyl), —C(O)(C₁-C₆ alkyl), C₆-C₁₄ aryl, C₁-C₉ heteroaryl, and C₃-C₈ cycloalkyl.

“Alkynyl” refers to a straight or branched chain unsaturated hydrocarbon containing at least one triple bond. Examples of C₂-C₆ alkynyl include, but are not limited to, acetylene, propyne, 1-butyne, 2-butyne, isobutyne, sec-butyne, 1-pentyne, 2-pentyne, isopentyne, 1-hexyne, 2-hexyne, 3-hexyne, and isohexyne. Examples of C₃-C₆ alkynyl include, but are not limited to, prop-2-ynyl, but-3-ynyl, but-2-ynyl, pent-4-ynyl, and hex-5-ynyl. Examples of C₃-C₈ alkynyl include, but are not limited to, prop-2-ynyl, but-3-ynyl, but-2-ynyl, pent-4-ynyl, hex-5-ynyl, hept-3-ynyl, 2-methylhex-3-ynyl, oct-4-ynyl, and 2-methylhept-3-ynyl. An alkynyl group may be unsubstituted or substituted with one or more of halogen, OH, —OC₁-C₆ alkyl, NH₂, (C₁-C₆ alkyl)NH, (C₁-C₆ alkyl)(C₁-C₆ alkyl)N—, —N(C₁-C₃ alkyl)C(O)(C₁-C₆ alkyl), —NHC(O)(C₁-C₆ alkyl), —NHC(O)H, —C(O)NH₂, —C(O)NH(C₁-C₆ alkyl), —C(O)N(C₁-C₆ alkyl)(C₁-C₆ alkyl), CN, C₁-C₆ alkoxy, C₁-C₆ alkyl, —C(O)OH, —C(O)O(C₁-C₆ alkyl), —C(O)(C₁-C₆ alkyl), C₆-C₁₄ aryl, C₁-C₉ heteroaryl, and C₃-C₈ cycloalkyl.

“Alkoxy” refers to the group R—O— where R is an alkyl group, as defined above. Exemplary C₁-C₆ alkoxy groups include but are not limited to methoxy, ethoxy, n-propoxy, 1-propoxy, n-butoxy and t-butoxy. An alkoxy group may be unsubstituted or substituted with one or more of halogen, OH, alkoxy, NH₂, (alkyl)amino-, di(alkyl)amino-, (alkyl)C(O)N(C₁-C₃ alkyl)-, (alkyl)carboxyamido-, HC(O)NH—, H₂NC(O)—, (alkyl)NHC(O)—, di(alkyl)NC(O)—, CN, C(O)OH, (alkoxy)carbonyl-, (alkyl)C(O)—, aryl, heteroaryl, cycloalkyl, haloalkyl, amino(C₁-C₆ alkyl)-, (alkyl)carboxyl-, or carboxyamidoalkyl-.

“Aryl” refers to an aromatic 6 to 14 membered hydrocarbon group. Examples of C₆-C₁₄ aryl include, but are not limited to, phenyl, α-naphthyl, β-naphthyl, biphenyl, anthryl, tetrahydronaphthyl, fluorenyl, indanyl, biphenylenyl, and acenanaphthyl. Examples of C₆-C₁₀ aryl include, but are not limited to, phenyl, α-naphthyl, β-naphthyl, biphenyl, and tetrahydronaphthyl. An aryl group may be unsubstituted or substituted with one or more of alkyl, alkenyl, halogen, haloalkyl, alkoxy, haloalkoxy, OH, hydroxyalkyl, cycloalkyl, O(hydroxyalkyl), —O-(alkyl)(hydroxyalkyl), —O(alkyl)C(O)OH, -(alkyl)(alkoxy)halogen, NH₂, aminoalkyl-, dialkylamino-, C(O)OH, —C(O)O(alkyl), —OC(O)(alkyl), —O(alkyl)N(alkyl)(alkyl), N-alkylamido-, —C(O)NH₂, (alkyl)amido-, NO₂, aryloxy, heteroaryloxy, (aryl)amido, (alkoxy)carbonyl-, (alkyl)amino, alkylcarboxyl-, (alkyl)carboxyamido-, (aryl)alkyl-, (aryl)amino-, aryloxy, cycloalkenyl, heteroaryl, aryl, (heteroaryl)alkyl-, heterocyclyl, oxo-heterocyclyl, dioxo-heterocyclyl, oxo-heteroaryl, —O-(heterocyclyl), heterocyclyl(alkyl)-, (hydroxyalkyl)NH—, (hydroxyalkyl)₂N, —S-(alkyl), —SO(alkyl), —SO₂(alkyl), SO₂NH₂, SO₂N(alkyl)₂, SO₂NH(alkyl), SO₂(alkyl), SO₂trifluoroalkoxy, S(alkyl), SO₂N(aryl)₂, SO₂N(heteroaryl)₂, SO₂N(cycloalkyl)₂, —NHC(O)(aryl), —C(O)NH(aryl), —NHC(O)(heteroaryl), —C(O)NH(heteroaryl), trifluoroalkyl, CN, trifluoroalkoxy, monofluoroalkyloxy, difluoroalkyloxy, N(alkyl)SO₂(alkyl), N(cycloalkyl)SO₂(alkyl), N(hydroxyalkyl)SO₂(alkyl), N(alkylamino)SO₂(alkyl), N(alkoxy)SO₂(alkyl), NH(alkyl)SO₂(alkyl)-phenyl, NHC(O)(alkyl), NHC(O)C(hydroxyalkyl)(alkyl), NHC(O)hydroxyalkyl, NHC(O)(cycloalkyl), N(alkyl)₂(heterocyclyl), NH(alkyl)-(heterocyclyl), -(alkyl)C(O)NH(alkyl), -alkylCN, -(alkyl)N(alkyl)₂, alkylC(O)NH₂-heterocyclyl, alkyl-NH₂-heterocyclyl, -alkyl-O—C(O)alkyl-(heterocyclyl), -alkoxy(heterocyclyl), O-cycloalkyl, O-alkyl-N(alkyl)₂, O-alkyl(heterocyclyl), OC(O)alkyl(heterocyclyl), C(O)NH(alkyl)-O-alkyl, C(O)(heterocyclyl), C(O)(cycloalkyl)heterocyclyl, C(O)(alkyl)(heterocyclyl), —CH₂(hydroxyalkyl-heterocyclyl), hydroxyalkyl(heterocyclyl), -alkyl(heterocyclyl), —CON(alkyl)₂, —CON(aryl)₂, —CON(heteroaryl)₂, —CON(cycloalkyl)₂, —CON(heterocyclyl)₂, N(alkyl)₂, N(COalkyl)₂, N(COcycloalkyl)₂, N(CO-heterocyclyl)₂, N(SO₂alkyl)₂, N(SO₂(alkyl)(hydroxyalkyl))₂, N(SO₂(alkyl)(alkoxyalkyl))₂, N(SO₂(alkyl)(alkylamino))₂, N(SO₂-aryl)₂, N(SO₂-heteroaryl)₂, N(SO₂-cycloalkyl)₂, N(SO₂-heterocyclyl)₂, N(COO-alkyl)₂, N(COO-cycloalkyl)₂, N(COO-heterocyclyl)₂, N(alkylcarbonyl)₂, N(hydroxyalkyl)₂, —O(alkyl)NH₂, —O(alkyl)N(COalkyl)₂, —O(alkyl)N(COcycloalkyl)₂, —O(alkyl)N(CO-heterocyclyl)₂, —O(alkyl)N(CONR¹⁰R¹¹)₂, —O(alkyl)N(SO₂(alkyl)(C₁-C₆ hydroxyalkyl))₂, —O(alkyl)N(SO₂(alkyl)(alkoxyalkyl))₂, —O(alkyl)N(SO₂(alkyl)(alkylamino))₂, —O(alkyl)N(SO₂-aryl)₂, —O(alkyl)N(SO₂-heteroaryl)₂, —O(alkyl)N(SO₂-cycloalkyl)₂, —O(alkyl)N(SO₂-heterocyclyl)₂, —O(alkyl)N(COO-alkyl)₂, —O(alkyl)N(COO-cycloalkyl)₂, —O(alkyl)N(COO-heterocyclyl)₂, —O(alkyl)N(alkylcarbonyl)₂, —O(alkyl)N(hydroxyalkyl)₂ or a Spiro substituent.

The term “bicycle” or “bicyclic” as used herein refers to a molecule that features two fused rings, which rings are a cycloalkyl, heterocyclyl, or heteroaryl. In one embodiment, the rings are fused across a bond between two atoms. The bicyclic moiety formed therefrom shares a bond between the rings. In another embodiment, the bicyclic moiety is formed by the fusion of two rings across a sequence of atoms of the rings to form a bridgehead. Similarly, a “bridge” is an unbranched chain of one or more atoms connecting two bridgeheads in a polycyclic compound. In another embodiment, the bicyclic molecule is a “spiro” or “spirocyclic” moiety. The spirocyclic group is a carbocyclic or heterocyclic ring which bound through a single carbon atom of the spirocyclic moiety to a single carbon atom of a carbocyclic or heterocyclic moiety. In one embodiment, the spirocyclic group is a cycloalkyl and is bound to another cycloalkyl. In another embodiment, the spirocyclic group is a cycloalkyl and is bound to a heterocyclyl. In a further embodiment, the spirocyclic group is a heterocyclyl and is bound to another heterocyclyl. In still another embodiment, the spirocyclic group is a heterocyclyl and is bound to a cycloalkyl.

“(Aryl)alkyl” refers to an alkyl group, as defined above, wherein one or more of the alkyl group's hydrogen atoms has been replaced with an aryl group as defined above. (C₆-C₁₄ aryl)alkyl- moieties include benzyl, benzhydryl, 1-phenylethyl, 2-phenylethyl, 3-phenylpropyl, 2-phenylpropyl, 1-naphthylmethyl, 2-naphthylmethyl and the like. An (aryl)alkyl group may be unsubstituted or substituted with one or more of halogen, CN, NH₂, OH, (alkyl)amino-, di(alkyl)amino-, (alkyl)C(O)N(alkyl)-, (alkyl)carboxyamido-, HC(O)NH—, H₂NC(O)—, (alkyl)NHC(O)—, di(alkyl)NC(O)—, CN, OH, alkoxy, alkyl, C(O)OH, (alkoxy)carbonyl-, (alkyl)C(O)—, aryl, heteroaryl, cycloalkyl, haloalkyl, amino(alkyl)-, (alkyl)carboxyl-, carboxyamidoalkyl-, or NO₂.

“(Alkoxy)carbonyl-” refers to the group alkyl-O—C(O)—. Exemplary (C₁-C₆ alkoxy)carbonyl- groups include but are not limited to methoxy, ethoxy, n-propoxy, 1-propoxy, n-butoxy and t-butoxy. An (alkoxy)carbonyl group may be unsubstituted or substituted with one or more of halogen, OH, NH₂, (alkyl)amino-, di(alkyl)amino-, (alkyl)C(O)N(alkyl)-, (alkyl)carboxyamido-, HC(O)NH—, H₂NC(O)—, (alkyl)NHC(O)—, di(alkyl)NC(O)—, CN, alkoxy, C(O)OH, (alkoxy)carbonyl-, (alkyl)C(O)—, aryl, heteroaryl, cycloalkyl, haloalkyl, amino(alkyl)-, (alkyl)carboxyl-, carboxyamidoalkyl-, or NO₂.

“(Alkyl)amido-” refers to a —C(O)NH— group in which the nitrogen atom of said group is attached to a C₁-C₆ alkyl group, as defined above. Representative examples of (C₁-C₆ alkyl)amido- include, but are not limited to, —C(O)NHCH₃, —C(O)NHCH₂CH₃, —C(O)NHCH₂CH₂CH₃, —C(O)NHCH₂CH₂CH₂CH₃, —C(O)NHCH₂CH₂CH₂CH₂CH₃, —C(O)NHCH(CH₃)₂, —C(O)NHCH₂CH(CH₃)₂, —C(O)NHCH(CH₃)CH₂CH₃, —C(O)NH—C(CH₃)₃ and —C(O)NHCH₂C(CH₃)₃.

“(Alkyl)amino-” refers to an —NH group, the nitrogen atom of said group being attached to a alkyl group, as defined above. Representative examples of (C₁-C₆ alkyl)amino- include, but are not limited to CH₃NH—, CH₃CH₂NH—, CH₃CH₂CH₂NH—, CH₃CH₂CH₂CH₂NH—, (CH₃)₂CHNH—, (CH₃)₂CHCH₂NH—, CH₃CH₂CH(CH₃)NH— and (CH₃)₃CNH—. An (alkyl)amino group may be unsubstituted or substituted on the alkyl moiety with one or more of halogen, NH₂, (alkyl)amino-, di(alkyl)amino-, (alkyl)C(O)N(alkyl)-, (alkyl)carboxyamido-, HC(O)NH—, H₂NC(O)—, (alkyl)NHC(O)—, di(alkyl)NC(O)—, CN, OH, alkoxy, alkyl, C(O)OH, (alkoxy)carbonyl-, (alkyl)C(O)—, aryl, heteroaryl, cycloalkyl, haloalkyl, amino(alkyl)-, (alkyl)carboxyl-, carboxyamidoalkyl-, or NO₂.

“Aminoalkyl-” refers to an alkyl group, as defined above, wherein one or more of the alkyl group's hydrogen atoms is replaced with —NH₂; one or both H of the NH₂ may be replaced by a substituent.

“Alkylcarboxyl-” refers to an alkyl group, defined above that is attached to the parent structure through the oxygen atom of a carboxyl (C(O)—O—) functionality. Examples of (C₁-C₆ alkyl)carboxyl- include acetoxy, propionoxy, propylcarboxyl, and isopentylcarboxyl.

“(Alkyl)carboxyamido-” refers to a —NHC(O)— group in which the carbonyl carbon atom of said group is attached to a C₁-C₆ alkyl group, as defined above. Representative examples of (C₁-C₆ alkyl)carboxyamido- include, but are not limited to, —NHC(O)CH₃, —NHC(O)CH₂CH₃, —NHC(O)CH₂CH₂CH₃, —NHC(O)CH₂CH₂CH₂CH₃, —NHC(O)CH₂CH₂CH₂CH₂CH₃, —NHC(O)CH(CH₃)₂, NHC(O)CH₂CH(CH₃)₂, —NHC(O)CH(CH₃)CH₂CH₃, —NHC(O)C(CH₃)₃ and —NHC(O)CH₂C(CH₃)₃.

“(Aryl)amino” refers to a radical of formula (aryl)-NH—, wherein aryl is as defined above. “(Aryl)oxy” refers to Ar—O— where Ar is an aryl group, as defined above. “Cycloalkyl” refers to a non-aromatic, saturated, partially saturated, monocyclic, bicyclic or polycyclic hydrocarbon 3 to 12 membered ring system. Representative examples of C₃-C₁₂ cycloalkyl include, but are not limited to, cyclopropyl, cyclopentyl, cycloheptyl, cyclooctyl, decahydronaphthalen-1-yl, octahydro-1H-inden-2-yl, decahydro-1H-benzo[7]annulen-2-yl, and dodecahydros-indacen-4-yl. Representative examples of C₃-C₁₀ cycloalkyl include, but are not limited to, cyclopropyl, cyclobutyl, cyclopentyl, cyclohexyl, cycloheptyl, cyclooctyl, decahydronaphthalen-1-yl, and octahydro-1H-inden-2-yl. Representative examples of C₃-C₈ cycloalkyl include, but are not limited to, cyclopropyl, cyclobutyl, cyclopentyl, cyclohexyl, cycloheptyl, cyclooctyl, and octahydropentalen-2-yl. A cycloalkyl may be unsubstituted or substituted with one or more of halogen, NH₂, CN, (alkyl)NH, (alkyl)(alkyl)N—, —N(alkyl)C(O)(alkyl), —NHC(O)(alkyl), —NHC(O)H, —C(O)NH₂, —C(O)NH(alkyl), —C(O)N(alkyl)(alkyl), CN, OH, alkoxy, alkyl, alkenyl, alkynyl, C(O)OH, —C(O)O(alkyl), —C(O) alkyl), aryl, heteroaryl, heterocyclyl, cycloalkyl, haloalkyl, perfluoroalkyl, perfluoroalkyloxy, aminoalkyl-, (alkyl)amido, alkylcarboxyl, (alkyl)carboxyamido, —OC(O)(alkyl), arylalkyl, aryloxy, (heteroaryl)oxy, arylcarboxyamidoalkyl-, heterocyclyl(alkyl), —O(hydroxyalkyl), —O(alkyl)(alkoxy), heteroaryl(alkyl)hydroxyalkyl, monofluoroalkyloxy, difluoroalkyloxy, SO₂NH₂, —S-alkyl, —S(O)alkyl, —S(O)₂alkyl, NO₂. Additionally, each of any two hydrogen atoms on the same carbon atom of the carbocyclic ring may be replaced by an oxygen atom to form an oxo (═O) substituent.

“Halo” or “halogen” refers to F, Cl, Br and I.

“C₁-C₆haloalkyl” refers to a C₁-C₆ alkyl group, as defined above, wherein one or more of the C₁-C₆ alkyl group's hydrogen atoms is replaced with F, Cl, Br, or I. Each substitution may be independently selected from F, Cl, Br, or I. Representative examples of C₁-C₆haloalkyl- include, but are not limited to, —CH₂F, —CCl₃, —CF₃, CH₂CF₃, —CH₂Cl, —CH₂CH₂Br, —CH₂CH₂I, —CH₂CH₂CH₂F, —CH₂CH₂CH₂Cl, —CH₂CH₂CH₂CH₂Br, —CH₂CH₂CH₂CH₂I, —CH₂CH₂CH₂CH₂CH₂Br, —CH₂CH₂CH₂CH₂CH₂I, —CH₂CH(Br)CH₃, —CH₂CH(Cl)CH₂CH₃, —CH(F)CH₂CH₃ and —C(CH₃)₂(CH₂Cl).

“Heteroaryl” refers to a monocyclic, bicyclic, or polycyclic aromatic ring system containing at least one ring atom selected from the heteroatoms oxygen, sulfur and nitrogen. Examples of C₁-C₉ heteroaryl include, without limitation, furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, benzoxazole, benzothiazole, benzofuran, benzisoxazole, benzimidazole, N-methylbenzimidazole, benzothiodiazole, azabenzimidazole, indazole, quinoxaline, quinazoline, quinoline, and isoquinoline. Bicyclic C₁-C₉ heteroaryl include those where a phenyl, pyridine, pyrimidine or pyridazine ring is fused to a 5 or 6-membered monocyclic heteroaryl ring having one or two nitrogen atoms in the ring, one nitrogen atom together with either one oxygen or one sulfur atom in the ring, or one O or S ring atom. Bicyclic heteroaryl also include those where a phenyl, pyridine, pyrimidine or pyridazine ring is fused to a 5 or 6-membered monocyclic heteroaryl ring or heterocyclyl ring, where in the second ring is either unsaturated, fully saturated or partially saturated ring having one or two nitrogen atoms in the ring, one nitrogen atom together with either one oxygen or one sulfur atom in the ring, or one O or S ring atom. If the 2^{nd} ring is either fully or partially saturated, one of the CH₂ group may be replaced by a C═O or SO₂ group as a part of the ring such as indolin-2-one and 3,4-dihydroquinolin-2(1H)-one. Examples of monocyclic C₁-C₄ heteroaryl include 2H-tetrazole, 3H-1,2,4-triazole, furan, thiophene, oxazole, thiazole, isoxazole, isothiazole, imidazole, and pyrrole. A heteroaryl group may be unsubstituted or substituted with one or more of C₁-C₆ alkyl, halogen, haloalkyl, OH, CN, hydroxyalkyl, NH₂, aminoalkyl-, dialkylamino-, C(O)OH, —C(O)O(alkyl), —OC(O)(alkyl), N-alkylamido-, —C(O)NH₂, (alkyl)amido-, —NO₂, (aryl)alkyl, alkoxy, aryloxy, heteroaryloxy, (aryl)amino, (alkoxy)carbonyl-, (alkyl)amido-, (alkyl)amino, aminoalkyl-, alkylcarboxyl-, (alkyl)carboxyamido-, (aryl)alkyl-, (aryl)amino-, cycloalkenyl, di(alkyl)amino-, heteroaryl, (heteroaryl)alkyl-, heterocyclyl, heterocyclyl(alkyl)-, (hydroxyalkyl)NH—, (hydroxyalkyl)₂N, —NHC(O)aryl, —C(O)NHaryl, —NHC(O)heteroaryl, —C(O)NH(heteroaryl), —N(alkyl)OSO₂(alkyl), N(alkyl)(SO₂alkyl), N(alkyl)SO₂(cycloalkyl), —O(alkyl)NH₂, —O(alkyl)N(alkyl)₂, —O(alkyl)N(C(O)alkyl)₂, —O(alkyl)N(O(alkyl)NH₂C(O)cycloalkyl)₂, —O(alkyl)N(O)heterocyclyl)₂, O(alkyl)N(SO₂alkyl)₂, —O(alkyl)N(SO₂(alkyl)(hydroxyalkyl))₂, —O(alkyl)N(SO₂(alkyl)(alkoxyalkyl)₂, —O(alkyl)N(SO₂(alkyl)(alkylamino))₂, —O(alkyl)N(SO₂aryl)₂, —O(alkyl)N(SO₂heteroaryl)₂, —O(alkyl)N(SO₂cycloalkyl)₂, —O(alkyl)N(SO₂heterocyclyl)₂, —O(alkyl)N(COOalkyl)₂, —O(alkyl)N(COOcycloalkyl)₂, —O(alkyl)N(COOheterocyclyl)₂, —O(alkyl)N(alkylcarbonyl)₂, —O(alkyl)N(hydroxyalkyl)₂, —O(alkyl)N(CONH₂)₂, —O(alkyl)N(alkyl)₂, —O(alkyl)N(aryl)₂, —O(alkyl)N(heteroaryl)₂, —O(alkyl)N(cycloalkyl)₂, —O(alkyl)N(heterocyclyl)₂ or a Spiro substituent.

“Heterocycle” or “heterocyclyl” refers to monocyclic, bicyclic, polycyclic, or bridged head molecules in which at least one ring atom is a heteroatom. A heterocycle may be saturated or partially saturated. Exemplary C₁-C₉ heterocyclyl include but are not limited to aziridine, oxirane, oxirene, thiirane, pyrroline, pyrrolidine, dihydrofuran, tetrahydrofuran, dihydrothiophene, tetrahydrothiophene, dithiolane, piperidine, 1,2,3,6-tetrahydropyridine-1-yl, tetrahydropyran, pyran, thiane, thiine, piperazine, azepane, diazepane, oxazine, 5,6-dihydro-4H-1,3-oxazin-2-yl, 2,5-diazabicyclo[2.2.1]heptane, 2,5-diazabicyclo[2.2.2]octane, 3,6-diazabicyclo[3.1.1]heptane, 3,8-diazabicyclo[3.2.1]octane, 6-oxa-3,8-diazabicyclo[3.2.1]octane, 7-oxa-2,5-diazabicyclo[2.2.2]octane, 2,7-dioxa-5-azabicyclo[2.2.2]octane, 2-oxa-5-azabicyclo[2.2.1]heptane-5-yl, 2-oxa-5-azabicyclo[2.2.2]octane, 3,6-dioxa-8-azabicyclo[3.2.1]octane, 3-oxa-6-azabicyclo[3.1.1]heptane, 3-oxa-8-azabicyclo[3.2.1]octan-8-yl, 5,7-dioxa-2-azabicyclo[2.2.2]octane, 6,8-dioxa-3-azabicyclo[3.2.1]octane, 6-oxa-3-azabicyclo[3.1.1]heptane, 8-oxa-3-azabicyclo[3.2.1]octan-3-yl, 2-methyl-2,5-diazabicyclo[2.2.1]heptane-5-yl, 1,3,3-trimethyl-6-azabicyclo[3.2.1]oct-6-yl, 3-hydroxy-8-azabicyclo[3.2.1]octan-8-yl-, 7-methyl-3-oxa-7,9-diazabicyclo[3.3.1]nonan-9-yl, 9-oxa-3-azabicyclo[3.3.1]nonan-3-yl, 3-oxa-9-azabicyclo[3.3.1]nonan-9-yl, 3,7-dioxa-9-azabicyclo[3.3.1]nonan-9-yl, 4-methyl-3,4-dihydro-2H-1,4-benzoxazin-7-yl, thiazine, dithiane, and dioxane. The contemplated heterocycle rings or ring systems have a minimum of 3 members. Therefore, for example, C₁ heterocyclyl radicals would include but are not limited to oxaziranyl, diaziridinyl, and diazirinyl, C₂ heterocyclyl radicals include but are not limited to aziridinyl, oxiranyl, and diazetidinyl, C₉ heterocyclyl radicals include but are not limited to azecanyl, tetrahydroquinolinyl, and perhydroisoquinolinyl. A heterocyclyl group may be unsubstituted or substituted with one or more of alkyl, halogen, alkoxy, haloalkyl, OH, hydroxyalkyl, —C(O)(hydroxyalkyl), NH₂, aminoalkyl-, dialkylamino-, C(O)OH, —C(O)O(alkyl), —OC(O)(alkyl), N-alkylamido-, —C(O)NH₂, (alkyl)amido-, —C(O)(alkyl)CN, (alkyl)CN, (aryl)alkyl, perfluoroalkyl, heteroaryl, heterocyclyl, or one or more —CH₂— of the ring are replaced by C═O

“Heterocyclyl(alkyl)-” refers to an alkyl group, as defined above, wherein one or more of the alkyl group's hydrogen atoms has been replaced with a heterocycle group as defined above. Heterocyclyl(C₁-C₆ alkyl)- moieties include 1-piperazinylethyl, 4-morpholinylpropyl, 6-piperazinylhexyl, and the like. A heterocyclyl(alkyl) group may be unsubstituted or substituted with one or more of halogen, NH₂, (alkyl)amino-, di(alkyl)amino-, (alkyl)C(O)N(alkyl)-, (alkyl)carboxyamido-, HC(O)NH—, H₂NC(O)—, (alkyl)NHC(O)—, di(alkyl)NC(O)—, CN, OH, alkoxy, alkyl, C(O)OH, (alkoxy)carbonyl-, (alkyl)C(O)—, 4- to 7-membered monocyclic heterocycle, aryl, heteroaryl, or cycloalkyl.

“Heteroaryl(alkyl)” refers to a heteroaryl which is attached to an alkyl group and the heteroaryl is defined above.

“Hydroxyalkyl” refers to an alkyl group, as defined above, wherein one or more of the alkyl group's hydrogen atoms is replaced with OH. Examples of C₁-C₆ hydroxyalkyl moieties include, for example, —CH₂OH, —CH₂CH₂OH, —CH₂CH₂CH₂OH, —CH₂CH(OH)CH₂OH, —CH₂CH(OH)CH₃, —CH(CH₃)CH₂OH and higher homologs.

“Alkoxyalkyl” refers to an alkyl group, as defined above, wherein one or more of the alkyl group's hydrogen atoms is replaced with OC₁-C₆ alkyl. Examples of C₁-C₆ hydroxyalkyl include, for example, —CH₂OCH₃, —CH₂CH₂OC₂H₅, —CH₂CH₂CH₂OCH(CH₃)₂, —CH₂CH(OMe)CH₂OMe, —CH₂CH(OC₂H₅)CH₃, —CH(CH₃)CH₂OCH₃ and higher homologs.

“Perfluoroalkyl” refers to an alkyl group, defined above, having two or more fluorine atoms. Examples of C₁-C₆ perfluoroalkyl- include CF₃, CH₂CF₃, CF₂CF₃ and CH(CF₃)₂. This may also be referred to as mono or difluorine substituted alkyl group such as CHF₂ or CH₂F.

“Alkoxyamino-” refers to an —O-alkyl group, defined above having —NH₂ or —O— alkyl group defined above having —N(alkyl)₂.

“Perfluoroalkoxy” refers to an alkyl group defined above bonded to an oxygen via an ether linkage, having two or more fluorine atoms.

A “subject” or “patient” is a mammal, e.g., a human or a veterinary patient or subject, e.g., a human, mouse, rat, guinea pig, dog, cat, horse, cow, pig, or non-human primate, such as a monkey, chimpanzee, baboon or gorilla.

Representative “pharmaceutically acceptable salts” include but are not limited to those of an acid or base. In one embodiment, the pharmaceutical salt is selected from among water-soluble and water-insoluble salts. The salt may be of an acid selected from, e.g., among acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, trifluoroacetic, and camphorsulfonic. The salt can also be of a base selected from, e.g., sodium, potassium, calcium, and ammonium. Optionally, a composition of the invention may contain both a pharmaceutically acceptable salt and the free base form of a compound of the invention.

A compound of the invention may also be a prodrug of formula IA or IB. Prodrugs of compounds of formula IA or IB may be prepared using various methods known to those skilled in the art. See, e.g., Rautio, Nature Reviews Drug Discovery, 7:255-270 (2008) and Ettmayer, J. Med. Chem., 47:2393-2404 (2004), which are hereby incorporated by reference. In the case of drugs containing a hydroxy moiety, acetyl and other ester analogs are contemplated for use as prodrugs. See, e.g., Beaumont, Current Drug Metabolism, 4:461-485 (2003), which is hereby incorporated by reference. In the case of drugs containing an amine moiety, prodrugs containing amides and carbamates are contemplated. See, e.g., Simplício, Molecules, 13:519-547 (2008), which is hereby incorporated by reference. As specific examples, (alkoxycarbonyloxy)alkyl carbamates, (acyloxy)alkyl carbamates, and (oxodioxolenyl)alkyl carbamates may be utilized as effective prodrug strategies for amines. See, e.g., Li, Bioorg. Med. Chem. Lett., 7:2909-2912 (1997); Alexander, J. Med. Chem., 34:78-81 (1991); Alexander, J. Med. Chem., 31:318-322 (1988); and Alexander, J. Med. Chem., 39:480-486 (1996), all of which are incorporated by reference herein.

The following abbreviations are used and have the indicated definitions: ACN is acetonitrile; DMSO is dimethylsulfoxide; DMF is N,N-dimethylformamide; TFA is trifluroroacetic acid; rt is room temperature; and THF is tetrahydrofuran.

The words “comprise”, “comprises”, and “comprising” are to be interpreted inclusively rather than exclusively. The works “consist”, “consisting”, and its variants, are to be interpreted exclusively, rather than inclusively.

As used herein, the term “about” means a variability of 10% from the reference given, unless otherwise specified.

Methods useful for making the compounds of Formula IA and IB are set forth in the Examples below and generalized in Schemes 1 to 9. One of skill in the art will recognize that Schemes 1-9 can be adapted to produce the compounds of Formulae IA and IB and pharmaceutically accepted salts of compounds of Formulae 1A and 1B according to the present invention. In the reactions described, reactive functional groups, such as hydroxy, amino, imino, thio or carboxy groups, where these are desired in the final product, may be protected to avoid unwanted reactions. Conventional protecting groups may be used in accordance with standard practice.

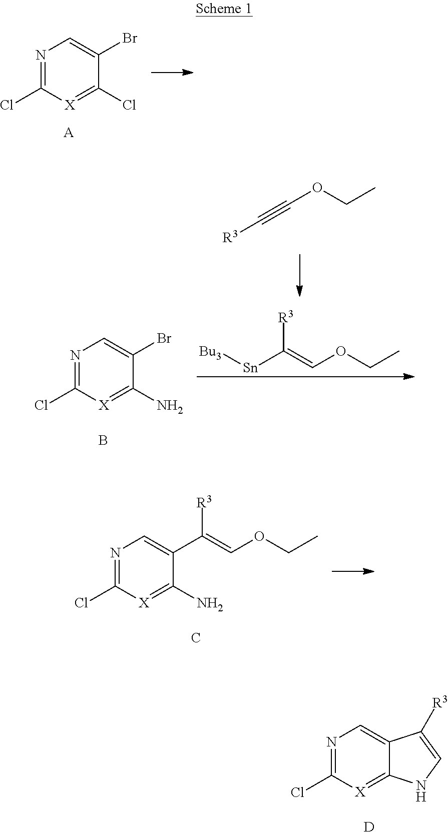

Scheme 1 provides the synthesis of intermediate D starting from compound A. Compound A is converted to the corresponding amino derivative B. In one embodiment, the conversion to compound B is performed using ammonia or liquid ammonia in an ether such as THF solution or DMF at reduced temperatures. The bromine in compound B is selectively replaced with a R³-substituted vinyl group to provide compound C. In one embodiment, compound C is prepared by reacting compound B with an R³-substituted vinyl tin compound. In a further embodiment, compound B is reacted with (Bu₃)₃SnC(R³)═C(H)OCH₂CH₃. This vinyl tin compound may be prepared by reacting R³—≡—OCH₂CH₃ with tributyl tin hydride in the presence of a radical initiator. In one embodiment, the radical initiator is azobisisobutyronitrile (AIBN). In a further embodiment, the vinyl tin compound is prepared at elevated temperatures. Compound C is typically prepared via a Pd catalyzed reaction. In a further embodiment, the catalyst is Pd(PPh₃)₄, PdCl₂(PPh₃)₂, PdCl₂(dppf), or Pd(OAc)₂. In yet another embodiment, the reaction is performed at elevated temperatures. Vinyl ether C is then hydrolyzed and cyclized using reagents that are known in the literature to provide compound D in the presence of an organic acid in a polar aprotic or protic solvent. In one embodiment, compound C is reacted with an acid such as HCl in the presence of an alcoholic solvent such as isopropyl alcohol). In a further embodiment, the reaction is performed at elevated temperatures.

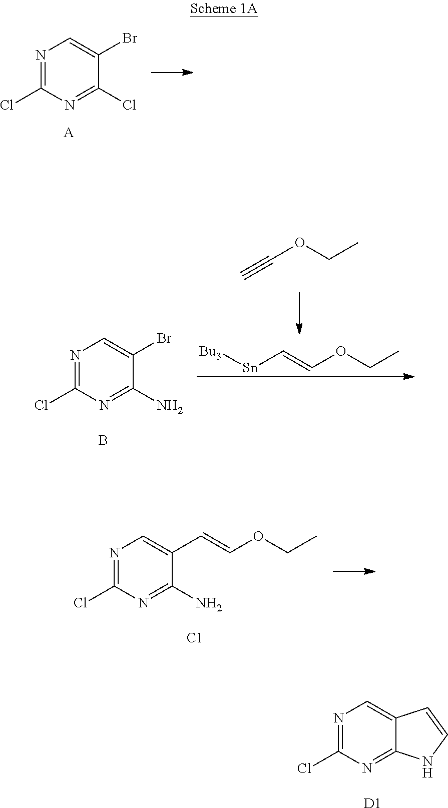

Scheme 1A provides the synthesis of intermediate 2-chloro-7H-pyrrolo[2,3-d]pyrimidine starting from 2,4-dichloro-5-bromo pyrimidine. 2,4-Dichloro-5-bromo pyrimidine is first converted to the corresponding amino compound via reaction with ammonia. In one embodiment, the reaction is performed in THF. The bromine in compound B is then selectively replaced with a vinyl group to provide compound C1. In one embodiment, compound B is reacted with a vinyl tin reagent in the presence of a Pd catalyst. In one embodiment, the Pd catalyst is Pd(PPh₃)₄. In another embodiment, the vinyl tin reagent is Bu₃SnCH═CHOCH₂CH₃ which may be prepared by reacting HC≡COCH₂CH₃ with Bu₃SnH in the presence of a radical initiator. In a further embodiment, the radical initiator is AIBN. Subsequent hydrolysis and cyclization of compound C1 provided compound D1. In one embodiment, compound C1 is reacted with an acid in the presence of an amine at elevated temperatures. In a further embodiment, the acid is HCl. In another embodiment, the amine is isopropylamine.

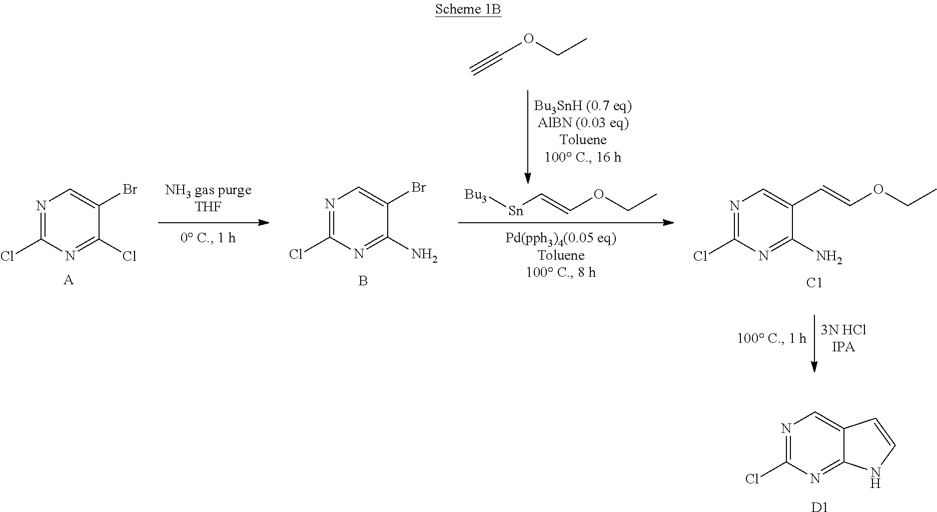

Scheme 1A provides the synthesis of intermediate 2-chloro-7H-pyrrolo[2,3-d]pyrimidine starting from 2,4-dichloro-5-bromo pyrimidine. 2,4-Dichloro-5-bromo pyrimidine is first converted to the corresponding amino compound via reaction with ammonia in THF. The bromine in compound B is then selectively replaced with a vinyl group to provide compound C1 using Pd(PPh₃)₄, Bu₃SnCH═CHOCH₂CH₃ (which is prepared by reacting HC≡COCH₂CH₃ with Bu₃SnH in the presence of AIBN). Subsequent hydrolysis and cyclization of compound C1 using HCl and isopropylamine at elevated temperatures provided compound D1.

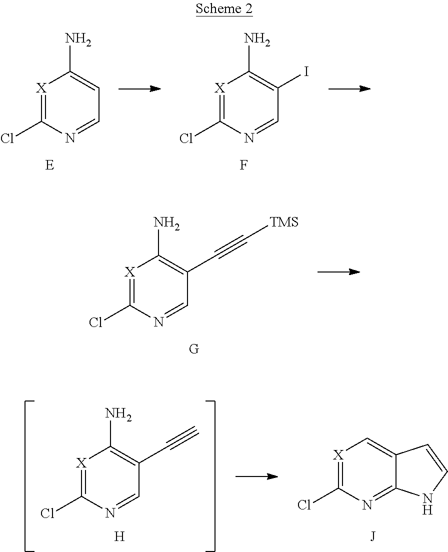

Scheme 2 provides the synthesis of intermediate compound J starting from compound E as indicated in Scheme 2. Initially, compound E is converted to the corresponding iodo derivative F. In one embodiment, the reaction is performed using iodine monochloride or iodine in THF or an organic aprotic solvent. The iodine atom in compound F is then selectively replaced by a TMS-acetylene group to provide compound G. In one embodiment, the reaction is performed in the presence of a Pd catalyst. Subsequent removal of the TMS group by an inorganic base provided compound intermediate H which cyclized to compound J in situ. In one embodiment, the reaction is performed using sodium or potassium tert-butoxide.

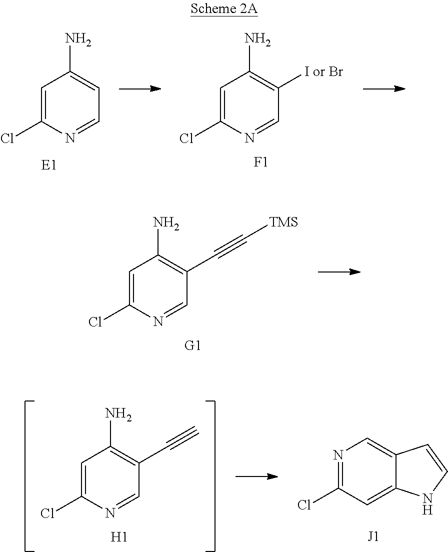

Scheme 2A provides the synthesis of intermediate compound J1 starting from compound E1 as indicated in Scheme 2A. Initially, compound E1 is converted to the corresponding iodo or bromo derivative F1. In one embodiment, the reaction is performed using iodine monochloride or iodine in THF or dioxane. The iodine atom in compound F1 is then selectively replaced by a TMS-acetylene group to provide compound G1. In one embodiment, the reaction is performed in the presence of a Pd catalyst. Subsequent removal of the TMS group by an inorganic acid provided compound intermediate H1 which cyclized to compound J1 in situ. In one embodiment, the reaction is performed using HCl.

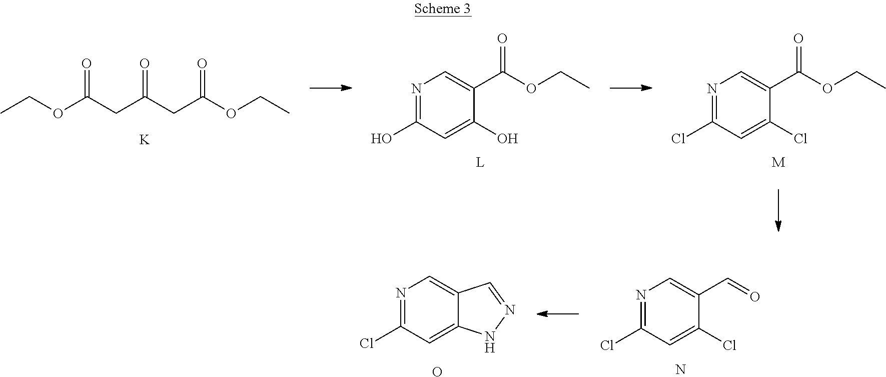

Scheme 3 provides the synthesis of intermediate O from commercially available diethyl acetone dicarboxylate K. In one embodiment, compound K is reacted with triethylorthoformate in acetic anhydride to provide 2,4-dihydroxy-5-carboethoxy pyridine L. In another embodiment, the reaction is performed at elevated temperatures. Compound K is then chlorinated to provide dichloro compound M. In one embodiment, the chlorination is performed using POCl₃. The corresponding dichloro derivative M is then converted to aldehyde N using a reducing agent. In one embodiment, the reducing agent is DiBAL-H. In another embodiment, the reduction is performed at reduced temperatures. Compound N is then reacted with hydrazine to provide compound O. In one embodiment, the reaction is performed at elevated temperatures.

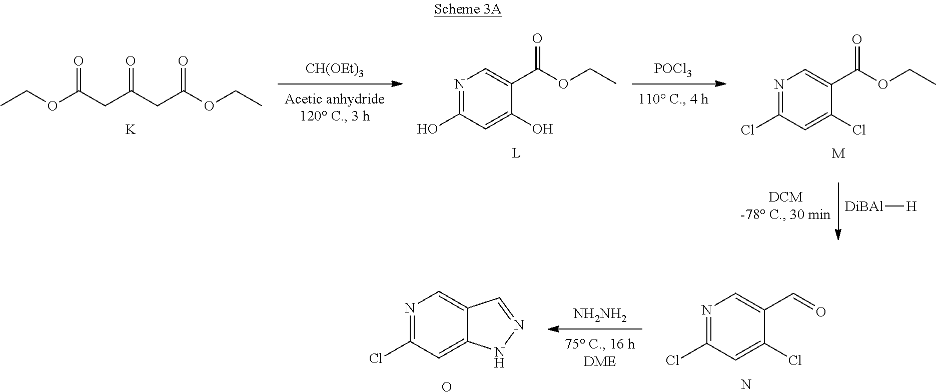

Scheme 3a provides the synthesis of intermediate O from commercially available diethyl acetone dicarboxylate K. In one embodiment, compound K is reacted with triethylorthoformate in acetic anhydride at elevated temperatures to provide 2,4-dihydroxy-5-carboethoxy pyridine L. Compound K is then chlorinated using POCl₃ to provide dichloro compound M. The corresponding dichloro derivative M is then converted to aldehyde N using DiBAL-H at reduced temperatures. Compound N is then reacted with hydrazine at elevated temperatures to provide compound O.

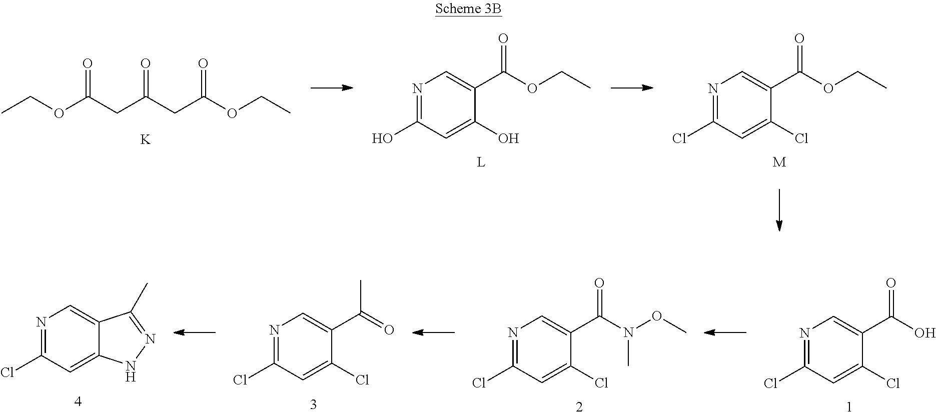

Scheme 3B provides the synthesis of intermediate 4 from intermediate compound M, which may be prepared as described in Scheme 3. The dichloro derivative M is then converted to acid 1 using an inorganic base such as LiOH, NaOH or KOH at room temperature. Compound N is then reacted with N-methoxy, N-methylamine in the presence of 1-ethyl-3-(3-dimethylaminopropyl)carbodiimide, hydrochloride EDC.HCl and N-hydroxybenzotriazole to provide compound 2. Compound 2 is then reacted with methyl magnesium bromide to provide ketone compound 3. Compound 3 is then reacted with hydrazine to provide compound 4.

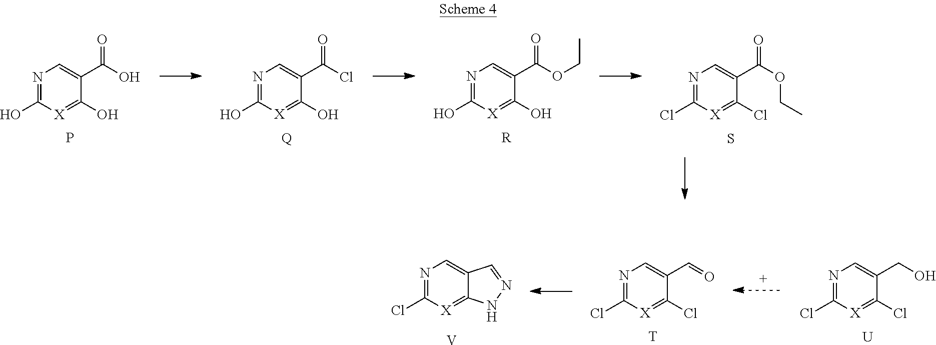

Scheme 4 provides the synthesis of the intermediate V from compound P. The acid group of compound P is esterified to compound R via acid chloride Q using reagents and techniques known to those skilled in the art. Compound R is then chlorinated to provide compound S. In one embodiment, the chlorination is performed using POCl₃. Compound S is converted to intermediate V via the aldehyde intermediate T using DiBAL-H in DCM at reduced temperature to provide a mixture of compounds U and T. Compound U is converted to compound T using Dess-Martin periodinane reagent or pyridinium chloro chromate (PCC). Subsequent reaction of compound T with hydrazine provides compound V.

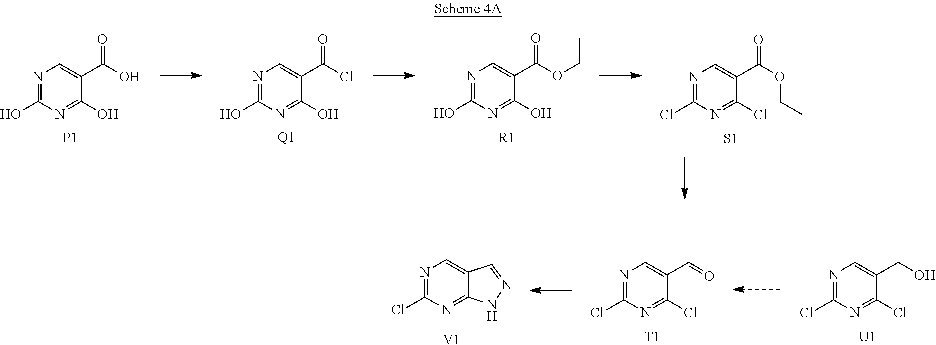

Scheme 4A provides the synthesis of 6-chloro-1H-pyrazolo[3,4-d]pyrimidine V1 from 2,4-dihydroxy-5-carbethoxy pyrimidine P1. The acid group of compound P1 is first esterified to compound R1 via acid chloride Q1 using reagents and techniques known to those skilled in the art. Compound R1 is then chlorinated to provide compound S. In one embodiment, the chlorination is performed using POCl₃. Compound S is converted to intermediate V1 via the aldehyde intermediate T1 using DiBAL-H in DCM at reduced temperature to provide a mixture of compounds U1 and T1. Compound U1 is converted to compound T1 using Dess-Martin periodinane reagent. Subsequent reaction of compound T1 with hydrazine provides compound V1.

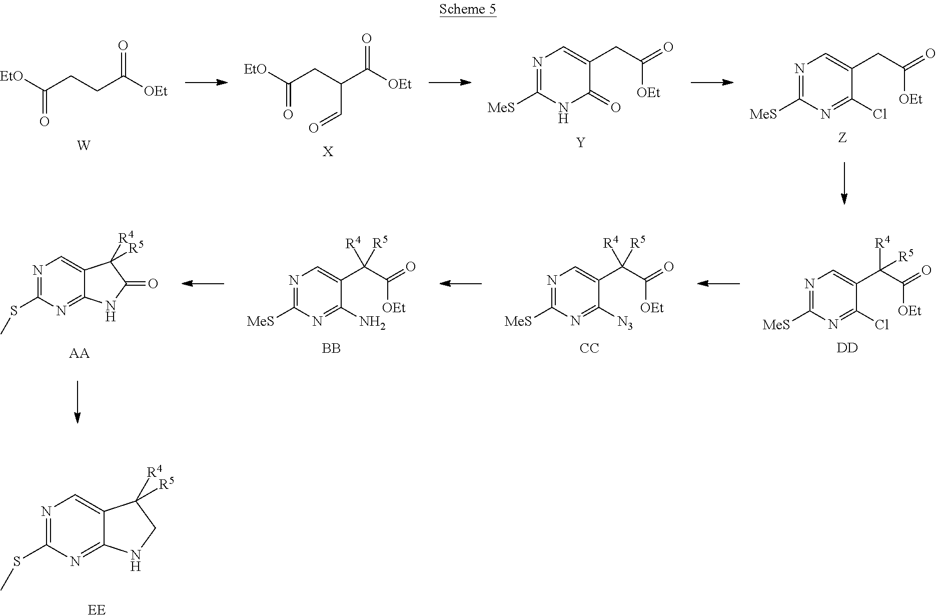

Scheme 5 provides the synthesis of intermediate compound EE starting from diethyl succinate W. Compound Y is prepared in two steps by formylating compound W to provide compound X. In one embodiment, the formylation is performed using sodium hydride. In another embodiment, the formylation is performed at reduced temperatures. Compound X is then reacted with thiourea to provide compound Y. In one embodiment, the thiourea is S-methyl thiourea semisulfate. Compound Y is then chlorinated to give compound Z. In one embodiment, the chlorination is performed using POCl₃. In another embodiment, the chlorination is performed at elevated temperatures. Compound Z is then R⁴/R⁵ substituted to provide compound DD. In one embodiment, compound Z is reacted with an R⁴/R⁵-substituted alkylating agent. In another embodiment, compound Z is reacted with methyl iodide. In a further embodiment, the R⁴/R⁵ substitution is performed in the presence of base such as NaH in DMF. The chloro group of compound DD is then displaced to provide compound CC. In one embodiment, compound DD is reacted with sodium azide to provide azido derivative CC. In another embodiment, the reaction is performed at elevated temperatures. The azide functional group of compound CC is then reduced to provide compound BB. In one embodiment, the reduction is performed using hydrogen and Pd/C. Compound BB is then cyclized using with a base such as K^{t}OBu to provide compound AA. In one embodiment, the reaction is performed at reduced temperatures. The amide carbonyl of compound AA is then reduced to provide the final intermediate compound EE. In one embodiment, the reduction is performed using LiAlH₄. In another embodiment, the reduction is performed at reduced temperatures.

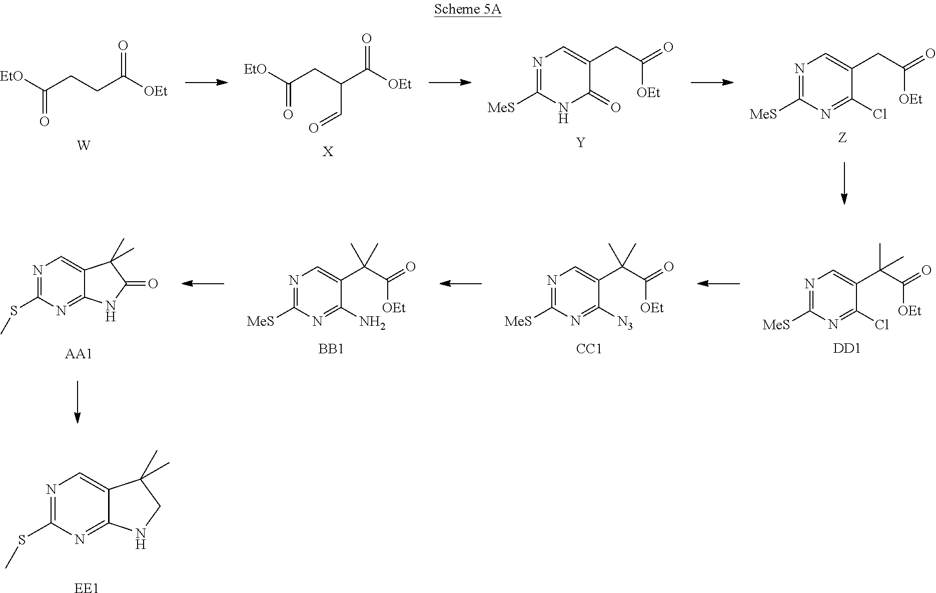

Scheme 5A provides the synthesis of intermediate 5,5-dimethyl-2-(methylthio)-6,7-dihydro-5H-pyrrolo[2,3-d]pyrimidine EE1 starting from diethyl succinate W. Compound Y is prepared in two steps by formylating compound W to provide compound X. In one embodiment, the formylation is performed using sodium hydride. In another embodiment, the formylation is performed at reduced temperatures. Compound X is then reacted with thiourea to provide compound Y. In one embodiment, the thiourea is S-methyl thiourea semisulfate. Compound Y is then chlorinated to give compound Z. In one embodiment, the chlorination is performed using POCl₃. In another embodiment, the chlorination is performed at elevated temperatures. Compound Z is then methylated to provide compound DD1. In one embodiment, compound Z is reacted with a methylating agent. In another embodiment, compound Z is reacted with methyl iodide. In a further embodiment, the methylation is performed in the presence of base such as NaH in DMF. The chloro group of compound DD1 is then displaced to provide compound CC1. In one embodiment, compound DD1 is reacted with sodium azide to provide azido derivative CC1. In another embodiment, the reaction is performed at elevated temperatures. The azide functional group of compound CC1 is then reduced to provide compound BB1. In one embodiment, the reduction is performed using hydrogen and Pd/C. Compound BB1 is then cyclized using with a base such as K^{t}OBu to provide compound AA1. In one embodiment, the reaction is performed at reduced temperatures. The amide carbonyl of compound AA1 is then reduced to provide the final intermediate compound EE1. In one embodiment, the reduction is performed using LiAlH₄. In another embodiment, the reduction is performed at reduced temperatures.

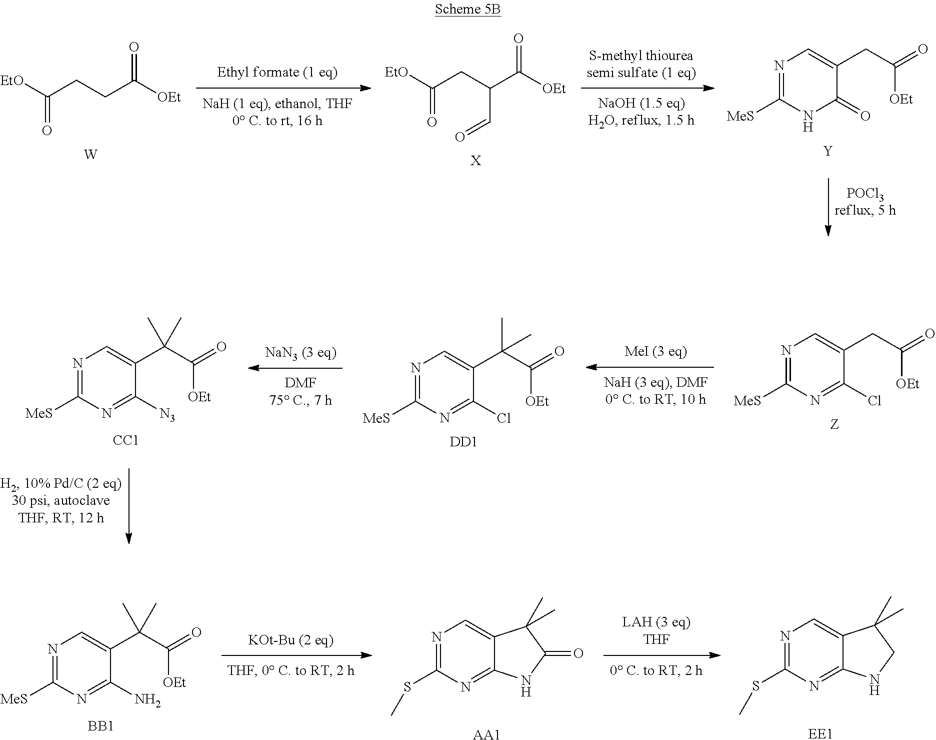

Scheme 5B provides the synthesis of intermediate 5,5-dimethyl-2-(methylthio)-6,7-dihydro-5H-pyrrolo[2,3-d]pyrimidine starting from diethyl succinate. Compound Y is prepared in two steps by formylating compound W using sodium hydride at reduced temperatures to provide compound X. Compound X is then reacted with S-methyl thiourea semisulfate to provide compound Y. Compound Y is then chlorinated using POCl₃ at elevated temperatures to give compound Z. Compound Z is then methylated using methyl iodide in the presence of NaH in DMF to provide compound DD1. The chloro group of compound DD1 is then displaced using sodium azide at elevated temperatures to provide azido compound CC1. The azide functional group of compound CC1 is then reduced using hydrogen and Pd/C to provide compound BB1. Compound BB1 is then cyclized using with K^{t}OBu at reduced temperatures to provide compound AA1. The amide carbonyl of compound AA1 is then reduced using LiAlH₄ at reduced temperatures to provide the final intermediate compound EE1.

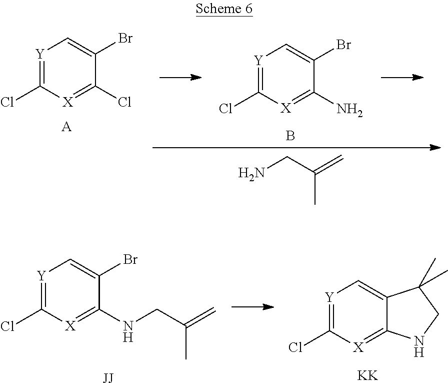

Scheme 6 provides an alternate way to synthesize chloro intermediate KK via a Heck reaction. Specifically, compound A is converted to its amino derivative. In one embodiment, compound A is reacted with ammonia. In another embodiment, ammonia gas at reduced temperatures is passed through a THF solution of compound A. Amino compound B is then alkylated to yield compound JJ. In one embodiment, the alkylation is performed using an allylbromide. In another embodiment, the alkylation is performed in the presence of NaH in DMF solution. In a further embodiment, the alkylation is performed at reduced temperatures. Compound JJ is then subjected to a Heck reaction to provide compound KK. In one embodiment, compound JJ is reacted with TBAB in the presence of a palladium catalyst. In another embodiment, the palladium catalyst is Pd(OAc)₂. In a further embodiment, the reaction is performed in the presence of a base such as triethylamine. In yet another embodiment, the reaction is performed in a solvent such as dimethylacetamide. In still a further embodiment, the reaction is performed at elevated temperatures.

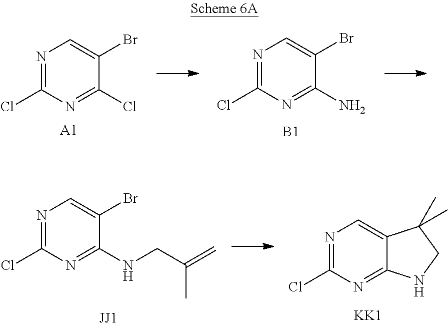

Scheme 6A provides an alternate way to synthesize chloro intermediate 2-chloro-5,5-dimethyl-6,7-dihydro-5H-pyrrolo[2,3-d]pyrimidine B1 via a Heck reaction. Specifically, compound A1 is converted to its amino derivative. In one embodiment, compound A1 is reacted with ammonia. In another embodiment, ammonia gas at reduced temperatures is passed through a THF solution of compound GG. Amino compound B1 is then alkylated to yield compound JJ1. In one embodiment, the alkylation is performed using an allylbromide. In another embodiment, the alkylation is performed in the presence of NaH in DMF solution. In a further embodiment, the alkylation is performed at reduced temperatures. Compound JJ1 is then subjected to a Heck reaction to provide compound KK1. In one embodiment, compound JJ1 is reacted with TBAB in the presence of a palladium catalyst. In another embodiment, the palladium catalyst is Pd(OAc)₂. In a further embodiment, the reaction is performed in the presence of a base such as triethylamine. In yet another embodiment, the reaction is performed in a solvent such as dimethylacetamide. In still a further embodiment, the reaction is performed at elevated temperatures.

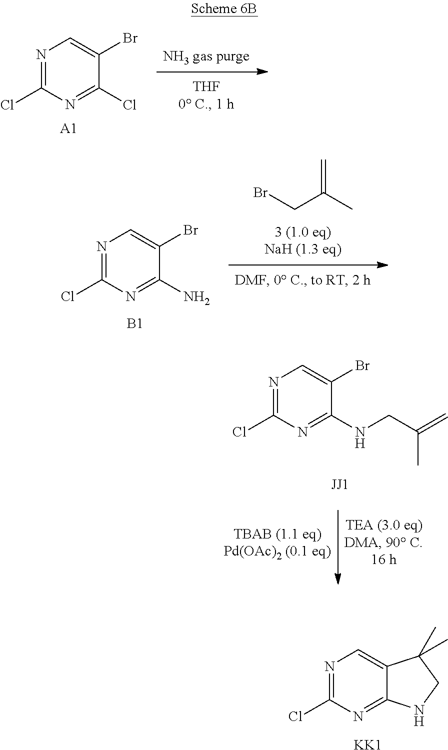

Scheme 6B provides the synthesis of 2-chloro-5,5-dimethyl-6,7-dihydro-5H-pyrrolo[2,3-d]pyrimidine KK1. Specifically, compound A1 is converted to its amino derivative by reacting compound A1 is reacted with ammonia by passing ammonia gas at reduced temperatures a THF solution of compound A1. Amino compound B1 is then alkylated using an allylbromide NaH in DMF solution at reduced temperatures to yield compound JJ1. Compound JJ1 is reacted with TBAB in the presence of Pd(OAc)₂, triethylamine, and dimethylacetamide at elevated temperatures.

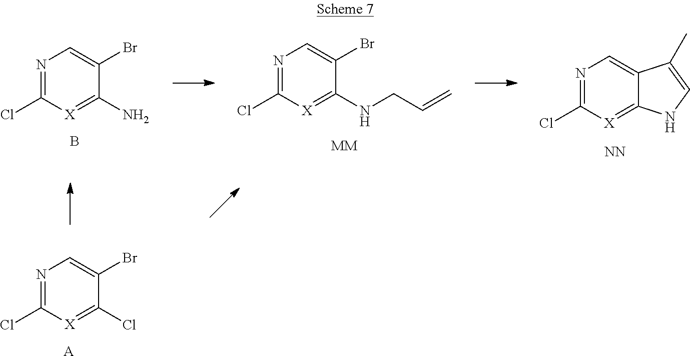

Scheme 7 provides the synthesis of the intermediate compound NN starting from compound B. Amino compound B is prepared as described above and is then alkylated to provide compound MM. In one embodiment, the alkylation is performed using allylbromide. In another embodiment, the alkylation is performed in the presence of NaH in DMF. In a further embodiment, the alkylation is performed at reduced temperatures. Compound MM is then subjected to Heck conditions to provide intermediate NN. In one embodiment, compound MM is reacted with TBAB. In another embodiment, the reaction is performed in the presence of an amine such as TEA. In a further embodiment, the reaction is performed in the presence of a palladium catalyst such as Pd(OAc)₂. In still another embodiment, the reaction is performed in the presence of a solvent such as dimethylacetamide. In yet a further embodiment, the reaction is performed at elevated temperatures.

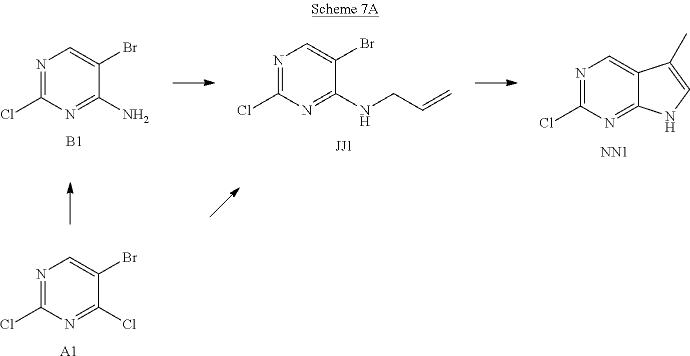

Scheme 7A provides the synthesis of the intermediate compound NN1 starting from compound B1. Amino compound B1 is prepared as described above and is then alkylated to provide compound JJ1. In one embodiment, the alkylation is performed using allylbromide. In another embodiment, the alkylation is performed in the presence of NaH in DMF. In a further embodiment, the alkylation is performed at reduced temperatures. Compound JJ1 is then subjected to Heck conditions to provide intermediate NN1. In one embodiment, compound JJ1 is reacted with TBAB. In another embodiment, the reaction is performed in the presence of an amine such as TEA. In a further embodiment, the reaction is performed in the presence of a palladium catalyst such as Pd(OAc)₂. In still another embodiment, the reaction is performed in the presence of a solvent such as dimethylacetamide. In yet a further embodiment, the reaction is performed at elevated temperatures.

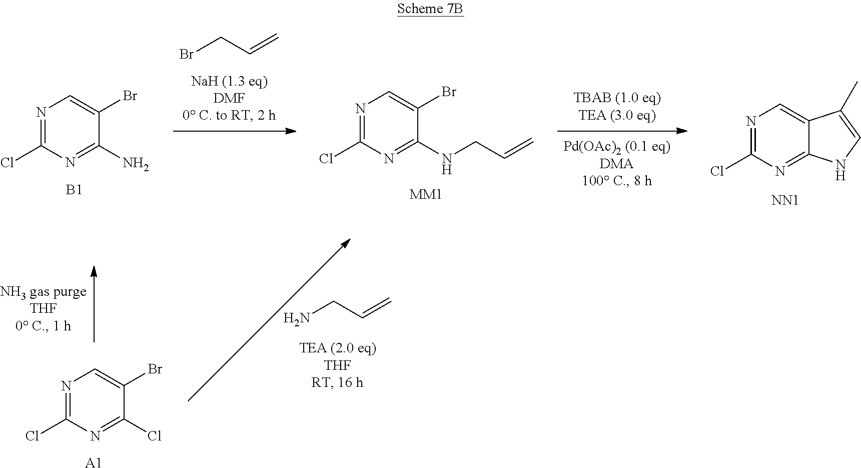

Scheme 7B provides the synthesis of the intermediate compound NN1 starting from compound B1. Amino compound B1 is prepared as described above and is then alkylated using allyl bromide in the presence of NaH in DMF at reduced temperatures to provide compound MM1. Compound MM1 is then subjected to Heck conditions to provide intermediate NN1. In one embodiment, compound MM1 is reacted with TBAB, in the presence of TEA, Pd(OAc)₂, and dimethylacetamide at elevated temperatures.

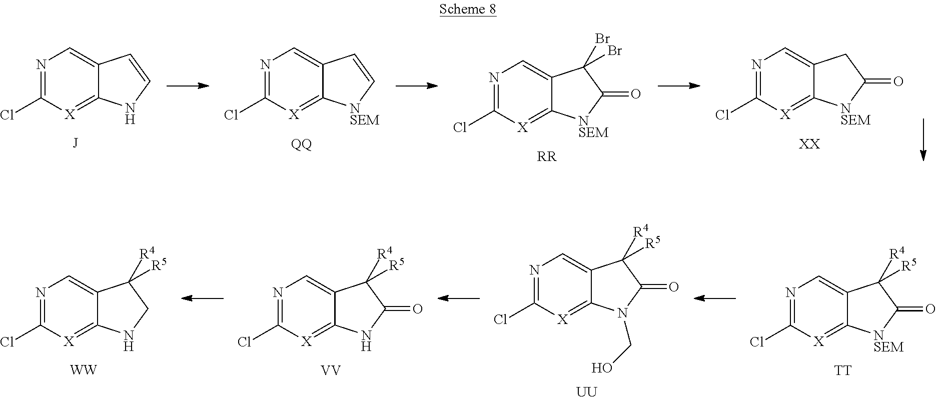

Scheme 8 provides the synthesis of intermediate compound WW starting from compound J (which is prepared as described in Scheme 2). Specifically, the indole nitrogen of compound J is protected. In one embodiment, the protection is performed using SEM-chloride. In another embodiment, the protection is performed in the presence of NaH in DMF. In a further embodiment, the protection is performed at reduced temperatures. Compound QQ is then brominated at the 3-position to provide compound RR. In one embodiment, the bromination is performed using N-bromosuccinimide. In another embodiment, the bromination is performed in n-butanol. The germinal bromines of compound RR are removed by a reductive bromination process to yield compound XX. In one embodiment, the reductive bromination is performed using Zn in the presence of AcOH or ammonium chloride. Compound XX is then R⁴/R⁵ alkylated to provide compound TT. In one embodiment, the alkylation is performed using an alkyl halide. In another embodiment, the alkylation is performed at reduced temperatures. In a further embodiment, the alkylation is performed in the presence of a base such as NaH, potassium carbonate, or cesium carbonate. The SEM protecting group of compound TT is then removed to provide compound UU. In one embodiment, the deprotection is performed using an acid such as TFA or hydrochloric acid. In a further embodiment, the deprotection is performed at reduced temperatures. Compound UU is then converted to compound VV using liquor ammonia in THF or DMF solvent. Finally, the reduction of compound VV provided compound WW. In one embodiment, the reduction is performed using a reducing agent such as LAH. In another embodiment, the reduction is performed at reduced temperatures.

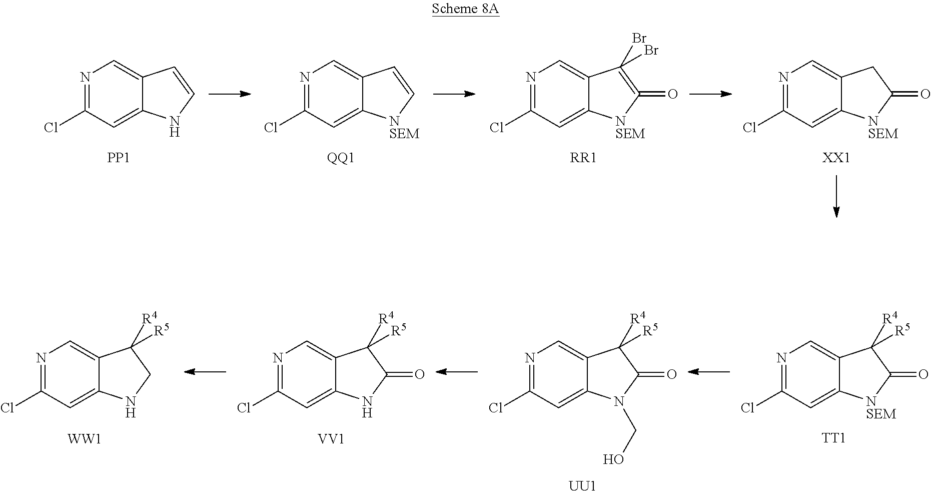

Scheme 8A provides the synthesis of intermediate compound WW1 starting from compound PP1. Specifically, the indole nitrogen of compound PP1 is protected. In one embodiment, the protection is performed using SEM-chloride. In another embodiment, the protection is performed in the presence of NaH in DMF. In a further embodiment, the protection is performed at reduced temperatures. Compound QQ1 is then brominated at the 3-position to provide compound RR1. In one embodiment, the bromination is performed using N-bromosuccinimide. In another embodiment, the bromination is performed in n-butanol. The germinal bromines of compound RR1 are removed by a reductive bromination process to yield compound XX1. In one embodiment, the reductive bromination is performed using Zn in the presence of AcOH. Compound XX1 is then R⁴/R⁵ alkylated to provide compound TT1. In one embodiment, the alkylation is performed using an alkyl halide. In another embodiment, the alkylation is performed at reduced temperatures. In a further embodiment, the alkylation is performed in the presence of a base such as NaH. The SEM protecting group of compound TT1 is then removed to provide compound UU1. In one embodiment, the deprotection is performed using an acid such as TFA. In a further embodiment, the deprotection is performed at reduced temperatures. Compound UU1 is then converted to compound VV1 using THF. Finally, the reduction of compound VV1 provided compound WW1. In one embodiment, the reduction is performed using a reducing agent such as LAH. In another embodiment, the reduction is performed at reduced temperatures.

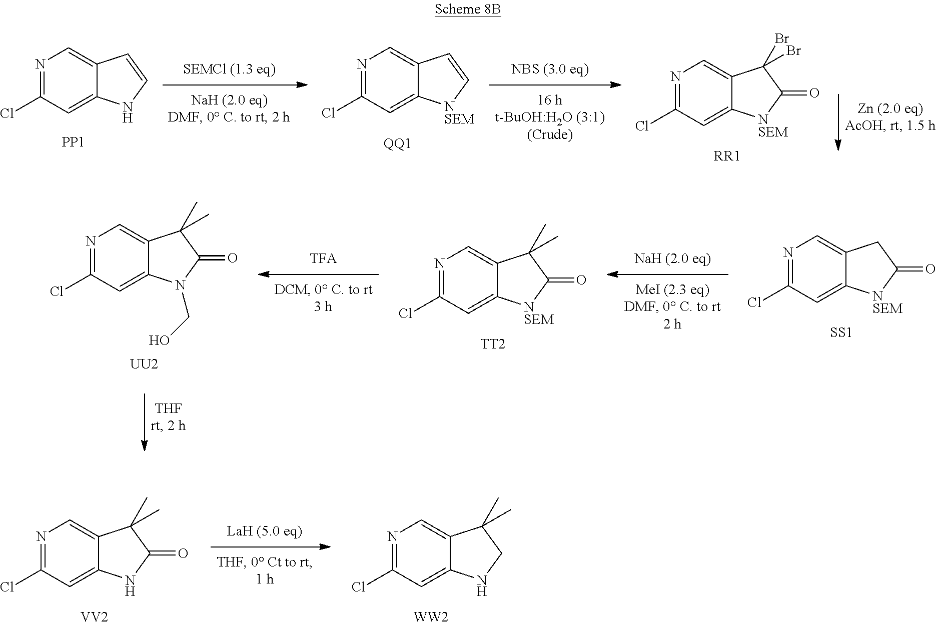

Scheme 8B provides the synthesis of 6-chloro-3,3-dimethyl-2,3-dihydro-1H-pyrrolo[3,2-c]pyridine WW2. Specifically, the indole nitrogen of compound PP1 is protected SEM-chloride in the presence of NaH in DMF at reduced temperatures. Compound QQ1 is then brominated at the 3-position using N-bromosuccinimide in n-butanol to provide compound RR1. The germinal bromines of compound RR1 are removed by a reductive bromination process using Zn in the presence of AcOH to yield compound XX1. Compound XX1 is then methylated using methyl iodide in the presence of NaH at reduced temperatures to provide compound TT2. The SEM protecting group of compound TT2 is then removed using TFA at reduced temperatures to provide compound UU2. Compound UU2 is then converted to compound VV2 using liquor ammonia in THF. Finally, the reduction of compound VV2 using LAH at reduced temperatures provides 6-chloro-3,3-dimethyl-2,3-dihydro-1H-pyrrolo[3,2-c]pyridine. In the compounds encompassed by structures IA or IB, when Z is —CH₂ or SO₂, these class molecules were synthesized from the appropriately substituted scaffold and an appropriately substituted B—CH₂—R₂ or B—SO₂—R₂, wherein B is a leaving group such as, but not limited to Cl, Br, I, tosylate or mesylate in the presence of an organic or inorganic base such as NaH, KH or LDA in suitable nonhydrous or hydrous organic solvents such as THF, DMF or acetone under heating condition. Several methods, such as Methods A-D, were followed depending upon the procedure used. Subsequently the chlorine in the pyrimidine or pyridine ring can be replaced by an appropriately substituted R¹—NH₂ using any one of Methods E-L. This may be accomplished by reacting the appropriately substituted fused heterocyclic scaffold with an appropriately substituted R²—NH₂ in the presence of Pd and a base in a polar aprotic solvent.

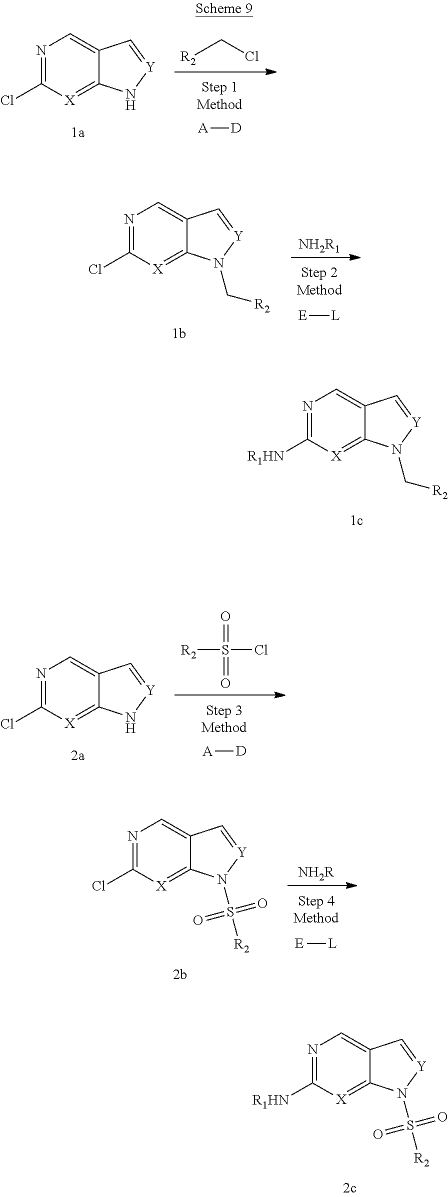

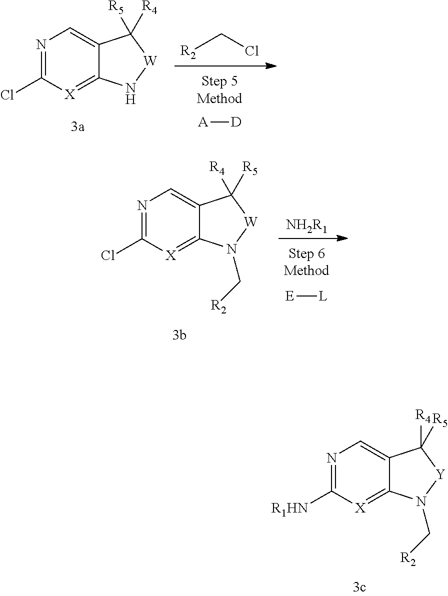

Scheme 9 provides a general procedure for N-alkylations and chloro displacements. Specifically, Scheme 9 sets forth the preparation of intermediate compounds 1c, 2c, and 3c. These intermediate compounds may be prepared using the procedures discussed above in Schemes 1-8, skill in the art, and reagents identified below for Methods A-N.

### Buchwald/Other Coupling Conditions:

Method A: K₂CO₃/DMF/Room Temperature

### Method B: NaH/DMF/RT

### Method C: Dimethylaminopyridine (DMAP)/Triethylamine/acetonitrile

Method D: CuI, K₃PO₄, trans-N,N-dimethyl cyclohexane, 100° C., dioxane, 4 h.
Method E: Pd₂(dba)₃, X-Phos, K₂CO₃, t-BuOH, 90° C.
Method F: Pd(OAc)₂, S-phos, K₂CO₃, dioxane, 100° C.
Method G: Pd₂(dba)₃, X-phos, K₂CO₃, DMF, 110° C.
Method H: Pd₂(dba)₃, xanthphos, dioxane, 100° C.
Method I: Trifluoroacetic acid, trifluoroethanol, 100° C., CEM MICROWAVE
Method J: Pd₂(dba)₃, S-phos, K₂CO₃, dioxane, 100° C.
Method K: Pd(OAc)₂, S-phos, Cs₂CO₃, dioxane, 100° C.
Method L: DIPEA, n-BuOH, 110° C.

### DeBoc Condition:

### Method M: TFA, DCM

Method N: 4N HCl in dioxane

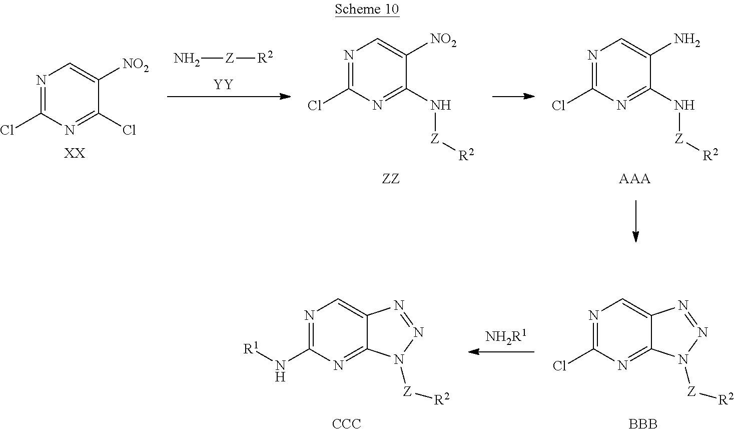

Scheme 10 provides the synthesis of triazolopyrimidine compound CCC encompassed by formula IA, i.e, Q is N. To a solution of 2,4-dichloro-5-nitropyrimidine XX in THF or a suitable protic or aprotic solvent, compound YY was added to provide compound ZZ. Compound ZZ was then reduced to provide compound AAA. In one embodiment, the reduction was performed using acetic acid and Fe, SnCl₂, or Zn dust and ammonium chloride. In another embodiment, the reduction was performed at elevated temperatures. Compound AAA was then cyclized to provide compound BBB. In one embodiment, the cyclization was performed using isoamyl nitrite. In another embodiment, the cyclization was performed in DMF or DMSO. In a further embodiment, the reaction was performed at elevated temperatures. Finally, chloro intermediate BBB is aminated. In one embodiment, compound BBB is reacted with an amine, a Pd catalyst, and X-Phos or xanthphos. In another embodiment, compound BBB is reacted with NH₂R¹. In a further embodiment, the reaction is performed in t-butanol or dioxane. In yet another embodiment, the reaction is performed under an inert atmosphere. In still a further embodiment, the reaction is performed at elevated temperatures. In another embodiment, the catalyst is Pd₂(dba)₃ or Pd(OAc)₂.

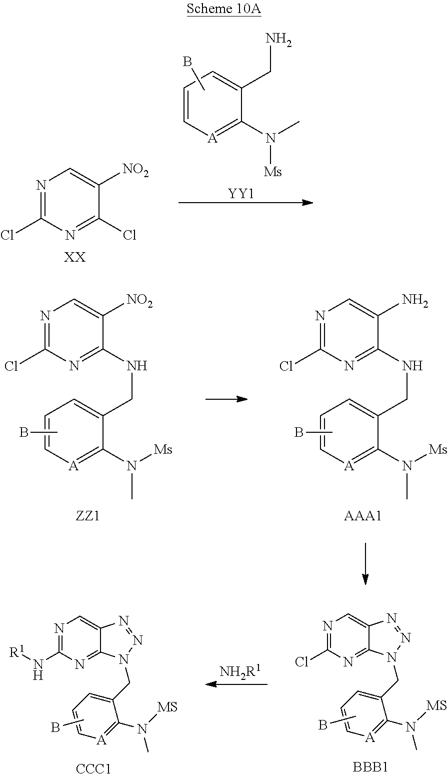

A is N or C—B where in B is H or any substituent.

Scheme 10A provides the synthesis of traizolopyrimidine compound CCC1 encompassed by formula IA, i.e, Q is N. To a solution of 2,4-dichloro-5-nitropyrimidine XX in THF or a polar aprotic solvent, compound YY1 was added to provide compound ZZ1. Compound ZZ1 was then reduced to provide compound AAA1. In one embodiment, the reduction was performed using acetic acid and Fe, SnCl₂, or Zn dust and ammonium chloride. In another embodiment, the reduction was performed at elevated temperatures. Compound AAA1 was then cyclized to provide compound BBB1. In one embodiment, the cyclization was performed using isoamyl nitrite. In another embodiment, the cyclization was performed in DMF or THF. In a further embodiment, the reaction was performed at elevated temperatures. Finally, chloro intermediate BBB1 is aminated. In one embodiment, compound BBB1 is reacted with an amine, a catalyst, and X-Phos or a suitable palladium catalyst. In another embodiment, compound BBB1 is reacted with NH₂R¹. In a further embodiment, the reaction is performed in t-butanol, DMF or dioxane. In yet another embodiment, the reaction is performed under an inert atmosphere. In still a further embodiment, the reaction is performed at elevated temperatures. In another embodiment, the catalyst is Pd₂(dba)₃ or palladium acetate.

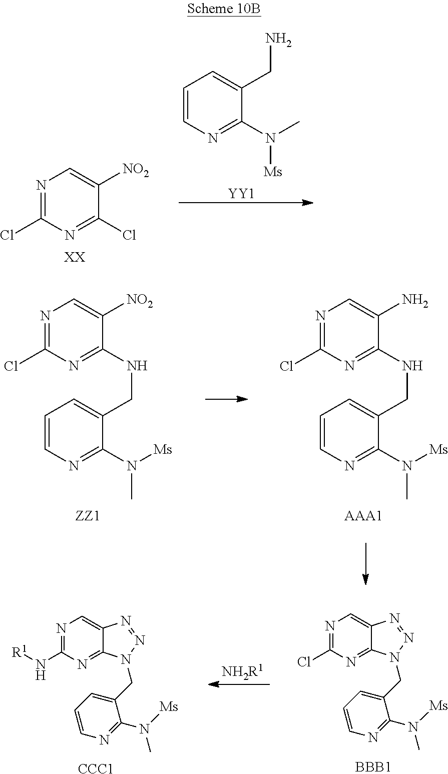

Scheme 10B also provides the synthesis of traizolopyrimidine compound BBB1 encompassed by formula IA, i.e, Q is N. To a solution of 2,4-dichloro-5-nitropyrimidine XX in THF or a polar aprotic solvent, compound YY1 was added to provide compound ZZ1. Compound ZZ1 was then reduced using acetic acid and Fe at elevated temperatures to provide compound AAA1. Compound AAA1 was then cyclized using isoamyl nitrite and DMF at elevated temperatures to provide compound BBB1. Finally, chloro intermediate BBB1 is reacted with an NH₂R¹, Pd₂(dba)₃, and X-Phos in t-butanol at elevated temperatures under an inert atmosphere.

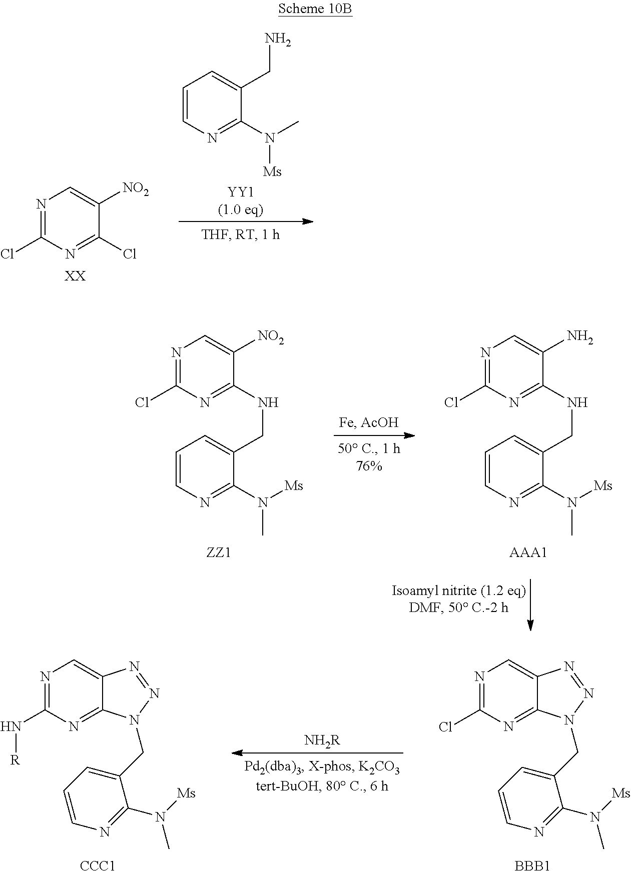

Scheme 10B provides the further synthesis of traizolopyrimidine compound CCC encompassed by formula IA, i.e, Q is N. Specifically, to a solution of 2,4-dichloro-5-nitropyrimidine XX (1.0 g) in THF (100 mL) at RT, compound YY1 (1.0 eq) was added and stirred for 1 h. After TLC showed completion, the solvent was distilled off under reduced pressure and the crude material was purified by flash chromatography on silica gel using EtOAc in hexane as eluent. The fractions with product ZZ1 were concentrated to obtain product 2 (0.8 g).

To a solution of N-(3-(((2-chloro-5-nitropyrimidin-4-yl)amino)methyl)pyridin-2-yl)-N-methylmethanesulfonamide ZZ1 (0.5 g) in acetic acid (50 mL) was added Fe (5 eq) and heated at 50° C. for 1 h. After TLC showed completion, the mixture was quenched with water and extracted with ethyl acetate. The organic layer was dried and concentrated give crude product that was purified by flash chromatography on silica gel using EtOAc in hexane as eluent. The fractions with product were concentrated to obtain compound AAA1 (0.35 g). In another embodiment, the reaction was performed using SnCl₂ or Zn dust and ammonium chloride.

To a solution of N-(3-(((5-amino-2-chloropyrimidin-4-yl)amino)methyl)pyridin-2-yl)-N-methylmethanesulfonamide AAA1 (1.02 g, 3.58 mmol) in DMF (12 mL) was added dropwise isoamyl nitrite (0.58 mL, 4.30 mmol) at rt. The mixture was then heated at 50° C. for 2 h, cooled and quenched with saturated solution of Na₂SO₃ (10 mL). Water (20 mL) was added to dissolve the precipitate, followed by EtOAc (60 mL). The organic layer was separated and the aqueous layer was extracted with EtOAc (4×60 mL). The combined organic extracts were washed with saturated solution of NaHCO₃ and brine, and dried over Na₂SO₄. The solvent was evaporated and the resulting residue was dried under high vacuum to give the crude product that was purified by flash chromatography on silica gel using EtOAc in hexane as eluent. The fractions with product were concentrated to obtain product BBB1.

A stirred mixture of chloro intermediate BBB1 (0.15 g, 1.0 eq), amine (0.9 eq) and K₂CO₃ (3.0 eq) in t-butanol (5.0 mL) in a vial was degassed using an argon balloon for 10 min. X-Phos (0.1 eq) and Pd₂(dba)₃ (0.05 eq) were added to this mixture and the solution continued degassing for another 10 min. The vial was closed and the contents heated at 90° C. for 3 h. After completion of the starting material, the \mixture was quenched with water (30 mL) and extracted with EtOAc (2×50 mL). The organic layer was dried over anhydrous Na₂SO₄, filtered and concentrated. The resulting residue was purified by flash chromatography (Biotage® Isolera™ purifier, 25 g column) using EtOAc in hexane as eluent. The desired product CCC1 eluted at 60% EtOAc in hexane. The fractions with product were concentrated to obtain product.

Pharmaceutical compositions useful herein comprise a compound of formula IA and/or IB in a pharmaceutically acceptable carrier optionally with other pharmaceutically inert or inactive ingredients. In another embodiment, a compound of formula IA and/or IB is present in a single composition. In a further embodiment, a compound of formula IA and/or IB is combined with one or more excipients and/or other therapeutic agents as described below.

The pharmaceutical compositions of the invention comprise an amount of a compound of formula IA and/or IB or a pharmaceutically acceptable salt thereof that is effective for regulating the FAK and/or Src pathway in a subject. Specifically, the dosage of the compound of formula IA and/or IB to achieve a therapeutic effect will depend on the formulation, age, weight and sex of the subject and route of delivery. It is also contemplated that the treatment and dosage of the compound of formula IA and/or IB may be administered in unit dosage form and that one skilled in the art would adjust the unit dosage form accordingly to reflect the relative level of activity. The decision as to the particular dosage to be employed (and the number of times to be administered per day) is within the discretion of the ordinarily-skilled physician, and may be varied by titration of the dosage to the particular circumstances to produce the desired therapeutic effect. In one embodiment, the therapeutically effective amount is about 0.01 mg/kg to 10 mg/kg body weight. In another embodiment, the therapeutically effective amount is less than about 5 g/kg, about 500 mg/kg, about 400 mg/kg, about 300 mg/kg, about 200 mg/kg, about 100 mg/kg, about 50 mg/kg, about 25 mg/kg, about 10 mg/kg, about 1 mg/kg, about 0.5 mg/kg, about 0.25 mg/kg, about 0.1 mg/kg, about 100 μg/kg, about 75 μg/kg, about 50 μg/kg, about 25 μg/kg, about 10 μg/kg, or about 1 μg/kg. However, the therapeutically effective amount of the compound of formula IA and/or IB can be determined by the attending physician and depends on the condition treated, the compound administered, the route of delivery, the age, weight, severity of the subject's symptoms and response pattern of the subject.

The therapeutically effective amounts may be provided on regular schedule, i.e., daily, weekly, monthly, or yearly basis or on an irregular schedule with varying administration days, weeks, months, etc. Alternatively, the therapeutically effective amount to be administered may vary. In one embodiment, the therapeutically effective amount for the first dose is higher than the therapeutically effective amount for one or more of the subsequent doses. In another embodiment, the therapeutically effective amount for the first dose is lower than the therapeutically effective amount for one or more of the subsequent doses. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner. The therapeutically effective amounts described herein refer to total amounts administered for a given time period; that is, if more than one compound of formula IA and/or IB or a pharmaceutically acceptable salt thereof is administered, the therapeutically effective amounts correspond to the total amount administered.

The pharmaceutical compositions comprising a compound of formula IA and/or IB may be formulated neat or with one or more pharmaceutical carriers for administration. The amount of the pharmaceutical carrier(s) is determined by the solubility and chemical nature of the compound of formula IA and/or IB, chosen route of administration and standard pharmacological practice. The pharmaceutical carrier(s) may be solid or liquid and may incorporate both solid and liquid carriers. A variety of suitable liquid carriers is known and may be readily selected by one of skill in the art. Such carriers may include, e.g., DMSO, saline, buffered saline, hydroxypropylcyclodextrin, and mixtures thereof. Similarly, a variety of solid carriers and excipients are known to those of skill in the art. The compounds of formula IA and/or IB may be administered by any route, taking into consideration the specific condition for which it has been selected. The compounds of formula IA and/or IB may, be delivered orally, by injection, inhalation (including orally, intranasally and intratracheally), ocularly, transdermally, intravascularly, subcutaneously, intramuscularly, sublingually, intracranially, epidurally, rectally, and vaginally, among others.

Although the compound of formula IA and/or IB may be administered alone, it may also be administered in the presence of one or more pharmaceutical carriers that are physiologically compatible. The carriers may be in dry or liquid form and must be pharmaceutically acceptable. Liquid pharmaceutical compositions are typically sterile solutions or suspensions. When liquid carriers are utilized for parenteral administration, they are desirably sterile liquids. Liquid carriers are typically utilized in preparing solutions, suspensions, emulsions, syrups and elixirs. In one embodiment, the compound of formula IA and/or IB is dissolved a liquid carrier. In another embodiment, the compound of formula IA and/or IB is suspended in a liquid carrier. One of skill in the art of formulations would be able to select a suitable liquid carrier, depending on the route of administration. The compound of formula IA and/or IB may alternatively be formulated in a solid carrier. In one embodiment, the composition may be compacted into a unit dose form, i.e., tablet or caplet. In another embodiment, the composition may be added to unit dose form, i.e., a capsule. In a further embodiment, the composition may be formulated for administration as a powder. The solid carrier may perform a variety of functions, i.e., may perform the functions of two or more of the excipients described below. For example, solid carrier may also act as a flavoring agent, lubricant, solubilizer, suspending agent, filler, glidant, compression aid, binder, disintegrant, or encapsulating material.

The composition may also be sub-divided to contain appropriate quantities of the compound of formula IA and/or IB. For example, the unit dosage can be packaged compositions, e.g., packeted powders, vials, ampoules, prefilled syringes or sachets containing liquids.

Examples of excipients which may be combined with one or more compound of formula IA and/or IB include, without limitation, adjuvants, antioxidants, binders, buffers, coatings, coloring agents, compression aids, diluents, disintegrants, emulsifiers, emollients, encapsulating materials, fillers, flavoring agents, glidants, granulating agents, lubricants, metal chelators, osmo-regulators, pH adjustors, preservatives, solubilizers, sorbents, stabilizers, sweeteners, surfactants, suspending agents, syrups, thickening agents, or viscosity regulators. See, for example, the excipients described in the “Handbook of Pharmaceutical Excipients”, 5^{th} Edition, Eds.: Rowe, Sheskey, and Owen, APhA Publications (Washington, D.C.), Dec. 14, 2005, which is incorporated herein by reference.

In one embodiment, the compositions may be utilized as inhalants. For this route of administration, compositions may be prepared as fluid unit doses using a compound of formula IA and/or IB and a vehicle for delivery by an atomizing spray pump or by dry powder for insufflation.

In another embodiment, the compositions may be utilized as aerosols, i.e., oral or intranasal. For this route of administration, the compositions are formulated for use in a pressurized aerosol container together with a gaseous or liquefied propellant, e.g., dichlorodifluoromethane, carbon dioxide, nitrogen, propane, and the like. Also provided is the delivery of a metered dose in one or more actuations.

In another embodiment, the compositions may be administered by a sustained delivery device. “Sustained delivery” as used herein refers to delivery of a compound of formula IA and/or IB which is delayed or otherwise controlled. Those of skill in the art know suitable sustained delivery devices. For use in such sustained delivery devices, the compound of formula IA and/or IB is formulated as described herein.

In addition to the components described above for use in the composition and the compound of formula IA and/or IB, the compositions may contain one or more medications or therapeutic agents which are used to treat solid tumors. In one embodiment, the medication is a chemotherapeutic. Examples of chemotherapeutics include those recited in the “Physician's Desk Reference”, 64^{th} Edition, Thomson Reuters, 2010, which is hereby incorporated by reference. Therapeutically effective amounts of the additional medication(s) or therapeutic agents are well known to those skilled in the art. However, it is well within the attending physician to determine the amount of other medication to be delivered.

The compounds of formula IA and/or IB and/or other medication(s) or therapeutic agent(s) may be administered in a single composition. However, the present invention is not so limited. In other embodiments, the compounds of formula IA and/or IB may be administered in one or more separate formulations from other compounds of formula IA and/or IB, chemotherapeutic agents, or other agents as is desired.

Also provided herein are kits or packages of pharmaceutical formulations containing the compounds of formula IA and/or IB or compositions described herein. The kits may be organized to indicate a single formulation or combination of formulations to be taken at each desired time.

Suitably, the kit contains packaging or a container with the compound of formula IA and/or IB formulated for the desired delivery route. Suitably, the kit contains instructions on dosing and an insert regarding the active agent. Optionally, the kit may further contain instructions for monitoring circulating levels of product and materials for performing such assays including, e.g., reagents, well plates, containers, markers or labels, and the like. Such kits are readily packaged in a manner suitable for treatment of a desired indication. For example, the kit may also contain instructions for use of a spray pump or other delivery device. Other suitable components to include in such kits will be readily apparent to one of skill in the art, taking into consideration the desired indication and the delivery route.

The compounds of formula IA and/or IB or compositions described herein can be a single dose or for continuous or periodic discontinuous administration. For continuous administration, a package or kit can include the compound of formula IA and/or IB in each dosage unit (e.g., solution, lotion, tablet, pill, or other unit described above or utilized in drug delivery), and optionally instructions for administering the doses daily, weekly, or monthly, for a predetermined length of time or as prescribed. When the compound of formula IA and/or IB is to be delivered periodically in a discontinuous fashion, a package or kit can include placebos during periods when the compound of formula IA and/or IB is not delivered. When varying concentrations of a composition, of the components of the composition, or the relative ratios of the compounds of formula IA and/or IB or agents within a composition over time is desired, a package or kit may contain a sequence of dosage units which provide the desired variability.

A number of packages or kits are known in the art for dispensing pharmaceutical agents for periodic oral use. In one embodiment, the package has indicators for each period. In another embodiment, the package is a labeled blister package, dial dispenser package, or bottle.

The packaging means of a kit may itself be geared for administration, such as an inhalant, syringe, pipette, eye dropper, or other such apparatus, from which the formulation may be applied to an affected area of the body, such as the lungs, injected into a subject, or even applied to and mixed with the other components of the kit.

The compositions of these kits also may be provided in dried or lyophilized forms. When reagents or components are provided as a dried form, reconstitution generally is by the addition of a suitable solvent. It is envisioned that the solvent also may be provided in another package.

The kits of the present invention also will typically include a means for containing the vials in close confinement for commercial sale such as, e.g., injection or blow-molded plastic containers into which the desired vials are retained. Irrespective of the number or type of packages and as discussed above, the kits also may include, or be packaged with a separate instrument for assisting with the injection/administration or placement of the composition within the body of an animal. Such an instrument may be an inhaler, syringe, pipette, forceps, measuring spoon, eye dropper or any such medically approved delivery means.

In one embodiment, a kit is provided and contains a compound of formula IA and/or IB. The compound of formula IA and/or IB may be in the presence or absence of one or more of the carriers or excipients described above. The kit may optionally contain instructions for administering the medication and the compound of formula IA and/or IB to a subject having a disease characterized by the dysregulation of the FAK and/or Src pathway.

In a further embodiment, a kit is provided and contains a compound of formula IA and/or IB in a second dosage unit, and one or more of the carriers or excipients described above in a third dosage unit. The kit may optionally contain instructions for administering the medication and the compound of formula IA and/or IB to a subject having a disease characterized by the dysregulation of the FAK and/or Src pathway.

The compounds described herein are useful in regulating conditions which are associated with the FAK and/or Src pathway. In one embodiment, such a disease is associated with abnormal cellular proliferation. The term “abnormal cellular proliferation” refers to the uncontrolled growth of cells which are naturally present in a mammalian body. In one embodiment, a disease which is characterized by abnormal cellular proliferation is cancer, including, without limitation, cancer of the prostate, head, neck, eye, mouth, throat, esophagus, bronchus, larynx, pharynx, chest, bone, lung, colon, rectum, stomach, bladder, uterus, cervix, breast, ovaries, vagina, testicles, skin, thyroid, blood, lymph nodes, kidney, liver, intestines, pancreas, brain, central nervous system, adrenal gland, or skin or a leukemia. In one embodiment, the disease characterized by abnormal cellular proliferation is cancer of the prostate.

The term “regulation” or variations thereof as used herein refers to the ability of a compound of formula IA and/or IB to inhibit one or more components of a biological pathway. In one embodiment, “regulation” refers to inhibition of FAK activity. In another embodiment, “regulation” refers to inhibition of Src activity. In a further embodiment, regulation refers to dual inhibition of FAK and Src activity.

In one embodiment, a novel cell line is provided which is useful for assessing and/or monitoring the activity of the compounds of the invention. Such cell lines may be included in a kit of the invention. Such a kit may be designed for performing an ELISA assay such as is described in Example 976 below, or in another method.

Thus, in one aspect, a stable cell line is provided which expresses a human FAK protein. This cell line is particularly well suited for use in assays for monitoring the activity of the compounds defined herein. In one example, a stable cell line is generated using human embryonic kidney (HEK293) cells (ATCC® CRL-1573™, 10801 University Boulevard, Manassas, Va. 20110 USA). However, other suitable cells may be obtained from the ATCC or other non-profit depositaries or commercial sources. In one embodiment, the nucleic acid sequence for focal adhesion protein (FAK, previously termed PTK) isoform A is selected for cloning into a suitable transfer vector. One suitable sequence is the nucleic acid sequence available at NCBI Reference Sequence: NM_153831.3, SEQ ID NO: 1 herein, which encodes a 1052 aa protein provided herein as SEQ ID NO:2. However, other sequences encoding the FAK isoform A protein may be selected, including such sequences which are naturally or artificially modified, e.g., natural variants or codon optimized variants of this sequence. A variety of codon optimization schema are known in the art. See, e.g., UpGene™ and Optimizer™, which are web-based optimization methods. Additionally, a number of commercial institutions perform codon optimization using proprietary schema, e.g., SignGen Laboratories, DNA2.0, OpenX, amongst others. In another embodiment, the coding sequence for a different FAK isoform may be selected, e.g., isoform 1, isoform 2, isoform 3, isoform 4, isoform 5 or isoform 6. See, e.g., provided in Uniprot ID: Q05397, Q05397-01 (isoform 1), Q05397-02 (isoform 2), Q05397-03 (isoform 3), Q05397-04 (isoform 4), Q05397-05 (isoform 5), and Q05397-06 (isoform 6). In these other embodiments, a coding sequence for the selected isoform is used for infection or transfection of the cell. The coding sequence for the selected FAK protein is cloned into a suitable vector for transfecting or infecting the cell to generate a FAK-expressing cell line. Suitable vectors may be generated using techniques known in the art. Alternatively, a suitable vector may be obtained from a non-profit, academic or commercial source. Examples of companies selling such vectors include, e.g., Sigma-Aldrich, Invitrogen, Promega, Life Technologies, amongst others. Once the vectors are generated, they are incubated with the target cells for a period of time sufficient to transfect the cells. These methods are known in the art and may further be provided by the manufacturer of the cloning vector. Typically, 48-96 hours, or about 72 hours, after transfection, cells are subcultured at various dilutions with fresh medium, which may optionally contain antibiotic where the cell has an antibiotic resistance gene. Cells may be replenished with selective medium as needed (e.g., every 2-5 days) until cell foci are identified in the FAK transfected cells and most of the cells died in the control plates. Cells from these foci are grown in the presence of selective media and the expression of FAK is confirmed. This may be done by Western Blotting or other suitable techniques. Over expression of FAK in the stably-transfected cells is quantified in a cell-based ELISA assay by determining the signal window (the signal window is the measure of separation between maximum and minimum controls in an assay that accounts for the amount of variability in the assay), with a signal window of 2 or above considered to be robust. The FAK stable cell line is maintained in the appropriate media and may be used in cell-based ELISA to assess autophosphorylation of FAK.

The compounds of formula IA and/or IB may be administered to the subject in need thereof together with radiation therapy. In one embodiment, the radiation is performed prior to administration of a compound of formula IA and/or IB. In another embodiment, the radiation is performed after administration of a compound of formula IA and/or IB. In a further embodiment, the radiation is performed concurrently with administration of a compound of formula IA and/or IB. The type and schedule for the required radiation may be selected by the ordinarily-skilled physician as determined by the particular disease being treated, patient, among other factors.

In one embodiment, methods for regulating the FAK and/or Src pathway are provided and comprise administering a therapeutically effective amount of a compound of formula IA and/or IB to a subject in need thereof. In one aspect, the regulation includes regulating the FAK and Src pathways.

In another desirable embodiment, methods for treating a disease characterized by abnormal cellular growth resulting from a dysregulated FAK and/or Src pathway are provided and comprise administering of a therapeutically effective amount of a compound of formula IA and/or IB to a subject in need thereof.

In a further desirable embodiment, methods for treating a condition treatable by inhibiting the FAK, Src, and/or pathway are provided and comprise administering a therapeutically effective amount of a compound of formula IA and/or IB to a subject in need thereof.

In yet another desirable embodiment, methods for inhibiting the FAK pathway, Src pathway, or a combination thereof are provided and comprise administering a compound of formula IA and/or IB to a subject in need thereof. In one aspect, both FAK and Src pathways are inhibited.

In still a further desirable embodiment, methods for treating a disease characterized by abnormal cellular proliferation resulting from a dysregulated FAK pathway, Src pathway, or a combination thereof are provided and comprise administering a therapeutically effective amount of a compound of formula IA and/or IB to a subject in need thereof. In one aspect, the disease is cancer. In another aspect, the disease is cancer is of the prostate, head, neck, eye, mouth, throat, esophagus, bronchus, larynx, pharynx, chest, bone, lung, colon, rectum, stomach, bladder, uterus, cervix, breast, ovaries, vagina, testicles, skin, thyroid, blood, lymph nodes, kidney, liver, intestines, pancreas, brain, central nervous system, adrenal gland, or skin or a leukemia. In a further aspect, the subject has at least one solid tumor.

In another desirable embodiment, methods of treating cancer are provided and comprise administering a compound of formula IA and/or IB to a subject in need thereof. In one aspect, the cancer is of the prostate, head, neck, eye, mouth, throat, esophagus, bronchus, larynx, pharynx, chest, bone, lung, colon, rectum, stomach, bladder, uterus, cervix, breast, ovaries, vagina, testicles, skin, thyroid, blood, lymph nodes, kidney, liver, intestines, pancreas, brain, central nervous system, adrenal gland, or skin or a leukemia. In a further aspect, the method comprises administering a chemotherapeutic agent. In another aspect, the method comprises administering radiation to said subject.

As described herein, a therapeutically effective amount of a compound when used for the treatment of cancer is an amount which may reduce the number of cancer cells or cause the number to remain relatively constant, reduce tumor size, inhibit metastasis, inhibit tumor growth and/or ameliorate one or more of the symptoms of the cancer. For cancer therapy, efficacy can be measured for example, by measuring tumor size or the presence and/or number of metastases, by assessing the time to disease progression and/or determining the response rate.

The following examples are illustrative only and are not intended to limit the present invention.

### EXAMPLES

### General Procedures for Synthesis of Intermediates 1b, 2b, and 3b (Scheme 9)

### General Procedure for Methods E, F, G, H, J and K

A stirred mixture of 1a, 2a or 3a (1.0 eq), amine (0.9 eq) and K₂CO₃ or Cs₂CO₃ (3.0 eq) in solvent (10.0 mL) in a vial was degassed using an argon balloon for 10 min. X-Phos, S-Phos or xanthphos (0.1 eq) and palladium catalyst (0.05 eq) were added to this mixture and degassing was continued for another 10 min. The vial was closed and the contents heated at the given temperature for 1-16 h while monitoring the progress by TLC and LCMS analysis. After completion of the starting material, the mixture was diluted with water and extracted with EtOAc (2×150 mL). The organic layer was dried over anhydrous Na₂SO₄, filtered and concentrated. The resulting residue was purified by Combiflash® chromatography using EtOAc in hexane as the eluent. The fractions with pure product were concentrated to obtain the final products.

### Procedure for Method I

### (R)-5,5-dimethyl-N-(4-(3-methylpiperazin-1-yl)phenyl)-7-(quinolin-8-ylsulfonyl)-6,7-dihydro-5H-pyrrolo[2,3-d]pyrimidin-2-amine (Example 470)

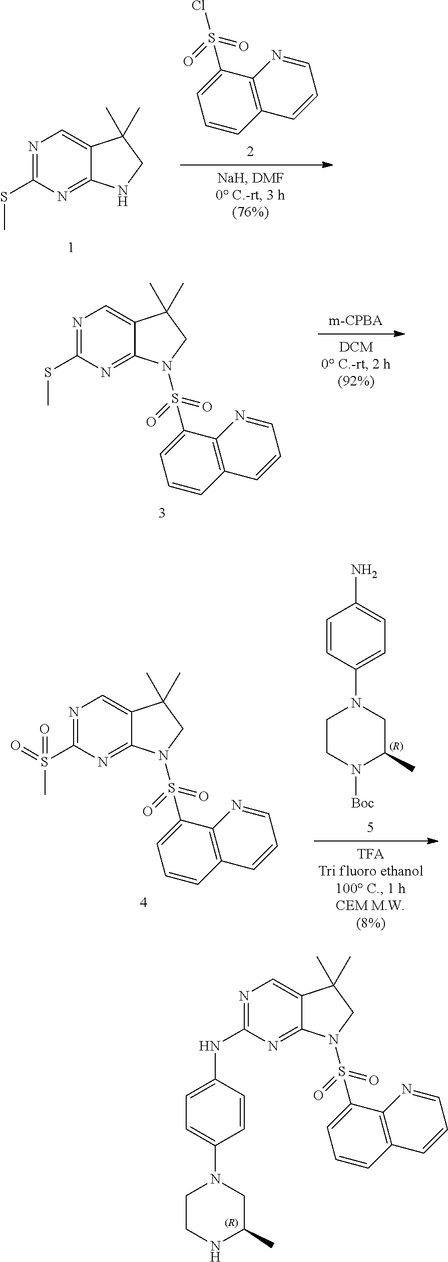

### Step 1: 8-((5, 5-dimethyl-2-(methylthio)-5H-pyrrolo[2,3-d]pyrimidin-7(6H)-yl)sulfonyl)quinoline

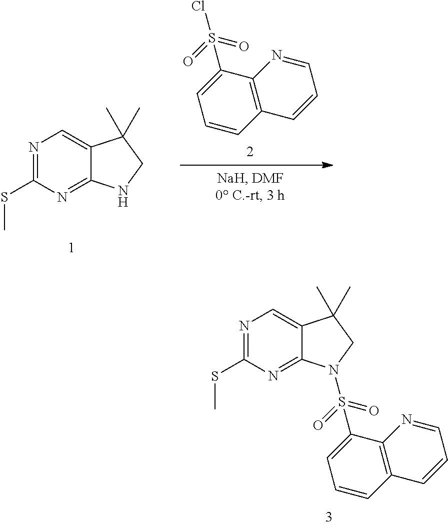 [SMILES: CSc1ncc2c(n1)NCC2(C)C ; O=S(=O)(Cl)c1cccc2cccnc12 ; CSc1ncc2c(n1)N(S(=O)(=O)c1cccc3cccnc13)CC2(C)C]

A solution of 5,5-dimethyl-2-(methylthio)-6,7-dihydro-5H-pyrrolo[2,3-d]pyrimidine (1 g, 5.128 mmol) in N,N′-dimethylformamide under nitrogen atmosphere was cooled to 0° C. Sodium hydride (410 mg, 10.256 mmol) was added to the mixture and stirred for 30 min at the same temperature. Quinoline-8-sulfonyl chloride (1.4 g, 6.153 mmol) was then added to the mixture and stirred for 2 h at rt. Progress of the reaction was followed by TLC (50% ethyl acetate/hexane). After completion of the reaction, it was quenched with saturated NH₄Cl solution (20 mL) and diluted with water (200 mL) and solid was collected by filtration and dried to give 8-((5,5-dimethyl-2-(methylthio)-5H-pyrrolo[2,3-d]pyrimidin-7(6H)-yl)sulfonyl)quinoline (Yield: 1.5 g, 76%). ¹H NMR (400 MHz, CDCl₃): δ 8.98-8.97 (d, 1H, J=4 Hz), 8.79-8.77 (d, 1H, J=7.2 Hz), 8.24-8.22 (d, 1H, J=8 Hz), 8.099-8.07 (d, 1H, J=11 Hz), 7.88 (s, 1H), 7.71-7.67 (t, 1H, J=16 Hz), 7.52-7.48 (m, 1H, J=8 Hz), 4.55 (s, 2H), 2.23 (s, 2H), 1.40 (s, 6H).

### Step 2: 8-((5, 5-dimethyl-2-(methylsulfonyl)-5H-pyrrolo[2,3-d]pyrimidin-7(6H)-yl)sulfonyl)quinoline

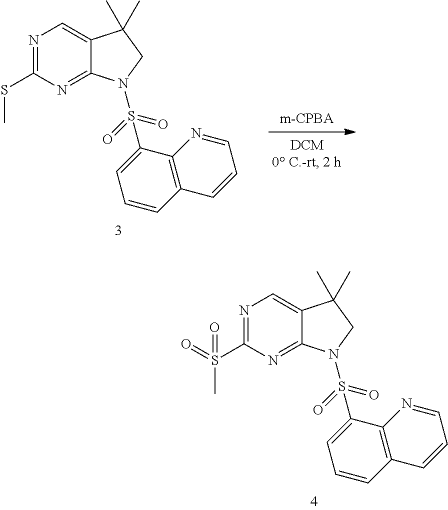 [SMILES: CSc1ncc2c(n1)N(S(=O)(=O)c1cccc3cccnc13)CC2(C)C ; CC1(C)CN(S(=O)(=O)c2cccc3cccnc23)c2nc(S(C)(=O)=O)ncc21]

To a solution of 8-((5, 5-dimethyl-2-(methylthio)-5H-pyrrolo[2,3-d]pyrimidin-7(6H)-yl) sulfonyl)quinoline (500 mg, 1.295 mmol) in DCM (10 mL) at 0° C. was added 3-chloro perbenzoic acid (5.58 g, 3.238 mmol). The mixture was stirred for 2 h at rt. Progress of the reaction was followed by TLC (50% ethyl acetate/hexane). After completion of the reaction, the mixture was diluted with saturated NaHCO₃ solution and extracted with DCM (3×20 mL). The combined organic layers were washed with water (20 mL), followed by brine (20 mL) and dried over Na₂SO₄. The organic layer was filtered and evaporated to give crude product, which was washed with diethyl ether (20 mL) to give 8-((5,5-dimethyl-2-(methylsulfonyl)-5H-pyrrolo[2,3-d]pyrimidin-7 (6H)-yl)sulfonyl)quinoline. This material was used for the next step without further purification (Yield: 500 mg, 92%).

### Step 3: (R)-5,5-dimethyl-N-(4-(3-methylpiperazin-1-yl)phenyl)-7-(quinolin-8-ylsulfonyl)-6,7-dihydro-5H-pyrrolo[2,3-d]pyrimidin-2-amine

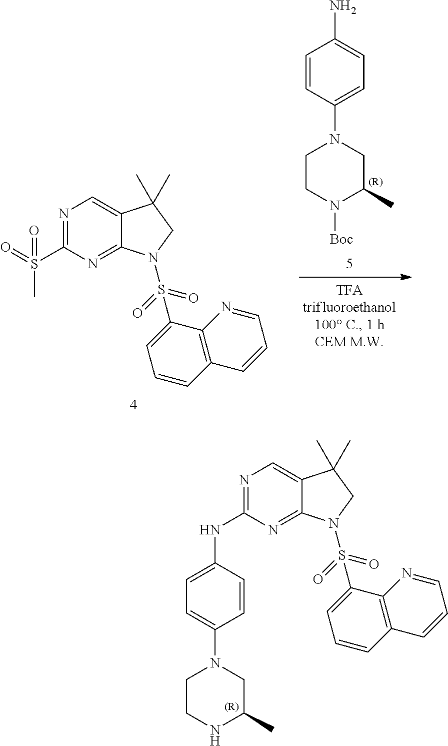

To a solution of 8-((5,5-dimethyl-2-(methylsulfonyl)-5H-pyrrolo[2,3-d]pyrimidin-7(6H)-yl)sulfonyl)quinoline (100 mg, 0.239 mmol) and (R)-tert-butyl 4-(4-aminophenyl)-2-methylpiperazine-1-carboxylate (84 mg, 0.287 mmol) in trifluoroethanol (5 mL) was added a catalytic amount of trifluoro acetic acid (5 drops). The mixture was irradiated in CEM microwave at 100° C. for 1 h. Progress of the reaction was followed by TLC (5% MeOH/DCM). After completion of the reaction, solvent was evaporated under reduced pressure to give crude residue, which was basified with saturated NaHCO₃ solution and extracted with ethyl acetate (2×30 mL). The combined organic layers were dried over Na₂SO₄, filtered and evaporated to give crude product. The resulting residue was purified by preparative TLC by using 5% MeOH/DCM to give (R)-5,5-dimethyl-N-(4-(3-methylpiperazin-1-yl)phenyl)-7-(quinolin-8-ylsulfonyl)-6,7-dihydro-5H-pyrrolo[2,3-d]pyrimidin-2-amine (Yield: 10 mg, 8%). ¹H NMR (400 MHz, DMSO-d6): δ 9.02 (s, 1H), 8.96 (s, 1H), 8.71-8.79 (d, 1H, J=7.2 Hz), 8.48-8.46 (d, 1H, J=8 Hz), 8.27-8.25 (d, 1H, J=8 Hz), 7.96 (s, 1H), 7.13-7.63 (m, 2H, J=4.4 Hz), 7.33-7.31 (t, 3H, J=9.6 Hz), 6.85-6.83 (d, 2H, J=8.4 Hz), 4.42 (s, 2H), 3.48-3.42 (t, 3H, J=9.6 Hz), 2.99-2.96 (d, 2H, J=11.6 Hz), 2.83 (s, 3H), 2.19-2.14 (t, 1H, J=10.8 Hz), 1.34 (s, 6H), 1.05-1.03 (d, 3H, J=6 Hz). [M+1] 530.3, Purity: 97.82%

### Procedure for Method L

A stirred mixture of 1a, 2a or 3a (1.0 eq), amine (0.95 eq), DIPEA (3.0 eq) in n-BuOH (10 mL) in a sealed vial was heated at 110° C. for 16 h. After TLC showed completion of starting material, the mixture was cooled to rt, poured into water and extracted with ethyl acetate (2×100 mL). The organic layer was dried over Na₂SO₄ and distilled off the solvent to get the crude material. The crude material was purified through flash chromatography (Combiflash® chromatograph, silica gel) using MeOH in DCM as the eluent and the pure fractions concentrate to provide the final products.

### A. 2-chloro-7H-pyrrolo[2,3-d]pyrimidine

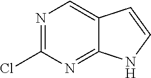

### Step 1: 5-Bromo-2-chloropyrimidin-4-amine

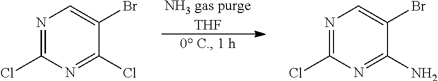 [SMILES: Clc1ncc(Br)c(Cl)n1 ; "0° C., 1 h" ; "NH3 gas purgeTHF" ; Nc1nc(Cl)ncc1Br]

A stirred solution of 5-bromo-2,4-dichloropyrimidine (5.0 g, 0.0220 mol, 1.0 eq, Combi blocks) in THF, at 0° C., was purged with NH₃ gas for 1 h while monitoring by TLC. After completion of the starting material, the mixture was quenched with water and extracted with EtOAc. The organic layer was dried over Na₂SO₄, filtered and concentrated to obtain off white solid 5-bromo-2-chloropyrimidin-4-amine (3.7 g, 80.78%). ¹H NMR (CDCl₃, 400 MHz): δ 8.237 (s, 1H), 5.696 (brs, 2H).

### Step 2: Tributyl(2-ethoxyvinyl)stannane

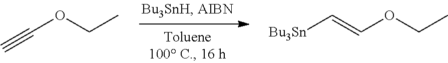

To a stirred solution of ethoxyethyne (3.0 g, 0.04285 mol, Alfa Aesar) and AIBN (0.210 g, 0.0012 mol) in toluene, tributyl tin hydride (8.72 g, 0.0299 mol) was added and the mixture heated to 100° C. for 16 h. Distilling off the solvent from mixture provided product tributyl(2-ethoxyvinyl)stannane as a brown gummy liquid (5.3 g, 34%) which was used in next step without purification.

### Step 3: 2-Chloro-5-(2-ethoxyvinyl)pyrimidin-4-amine

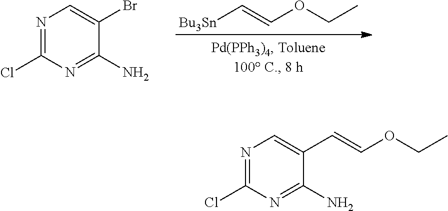

A stirred solution of 5-bromo-2-chloropyrimidin-4-amine (2.5 g, 0.0120 mol, 1.0 eq), tributyl(2-ethoxyvinyl)stannane (5.2 g, 0.0144 mol, 1.2 eq) in toluene in a two neck round bottomed flask was degassed using an argon balloon for 30 min and Pd(PPh₃)₄ (0.694 g, 0.05 eq) was added. The mixture was heated at 100° C. for 8 h while monitoring by TLC. After completion of starting material, the mixture was cooled to rt, quenched with water and extracted with EtOAc. The organic layer was dried over Na₂SO₄, filtered and concentrated to obtain brown gummy liquid 2-chloro-5-(2-ethoxyvinyl)pyrimidin-4-amine. This crude product was directly used in the next step without further purification (3.0 g). LCMS calculated for (M) 199.051 and found (M+H) 200.01.

### Step 4: 2-Chloro-7H-pyrrolo[2,3-d]pyrimidine

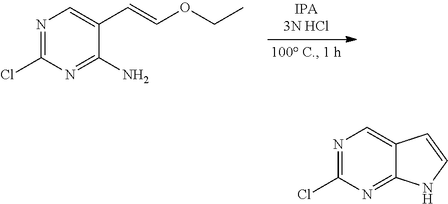

To a stirred solution of 2-chloro-5-(2-ethoxyvinyl)pyrimidin-4-amine (3.0 g, 0.0150 mol, 1.0 eq), in IPA in a two neck round bottomed flask, 3N HCl was added and the contents heated at 100° C. for 1 h while monitoring by TLC. After completion of starting material, the solvent was evaporated from the mixture to obtain residue. This residue was suspended in water and extracted with EtOAc (3×150 mL). The organic layer was dried over Na₂SO₄, filtered and concentrated to obtain the compound. This compound was purified by Combiflash® column chromatography using 100-200 silica gel with 30% EtOAc in hexane as the eluting solvent to obtain product 2-chloro-7H-pyrrolo[2,3-d]pyrimidine as yellow solid (0.680 g, 29.56%). ¹H NMR (DMSO-d₆, 400 MHz): δ 12.357 (brs, 1H), 8.918 (s, 1H), 7.614 (t, 1H), 6.644 (t, 1H).

### B. 6-Chloro-1H-pyrrolo[3,2-c]pyridine

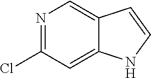

### Step 1: 2-Chloro-5-iodopyridin-4-amine

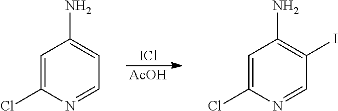

To 2-chloro-4-amino pyridine (33 g, 1 eq), in a 2-neck 2 L round bottomed flask, acetic acid (1.5 L) was added and stirred for 10 min. To this mixture, potassium acetate (50 g, 2 eq) was added and stirred for 10 min. Iodine monochloride (50 g, 1.2 eq) was added slowly to the mixture and the contents refluxed at 120° C. for 4 h while monitoring by TLC. After completion, the acetic acid was distilled off under vacuum and the residue was basified with NaHCO₃ and poured into water. The aqueous mixture was extracted with EtOAc (2×500 mL) and the organic layer was dried over anhydrous sodium sulphate, filtered and concentrated to obtain crude product. The crude was purified by column chromatography (silica gel, 60-120#) using EtOAc in hexane as the eluent. The desired product eluted at 10% EtOAc in hexane. The fractions with product were concentrated to obtain product 2-chloro-5-iodopyridin-4-amine as white solid (23 g, 45%). ¹H NMR (DMSO-d₆, 400 MHz): δ 8.190 (s, 1H), 6.625 (s, 1H), 6.517 (brs, 2H).

### Step 2: 2-Chloro-5-((trimethylsilyl)ethynyl)pyridin-4-amine

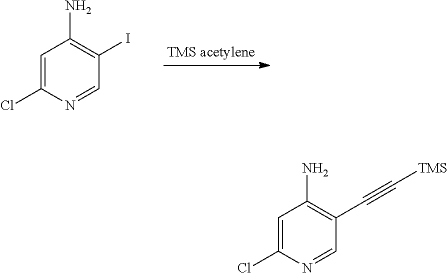 [SMILES: Nc1cc(Cl)ncc1I ; C[Si](C)(C)C#Cc1cnc(Cl)cc1N]

To 2-chloro-5-iodopyridin-4-amine (23 g, 1.0 eq) in a 500 mL round bottomed flask under nitrogen atmosphere, triethylamine (300 mL) and CuI (0.187 g, 0.01 eq) were added and degassed with argon for 15 min. To this mixture, PdCl₂(PPh₃)₂ (0.69 g, 0.01 eq) was added and degassed again for 10 min. Trimethylsilyl acetylene (13.3 g, 1.5 eq) was added to the mixture and stirred at rt for 16 h. After completion, the mixture was poured into water (500 mL) and extracted with EtOAc (2×500 mL). The organic layer was washed with brine solution (200 mL), dried over anhydrous sodium sulphate, filtered and concentrated to obtain the crude material. The crude material was purified by column chromatography (silica gel, 60-120#) using EtOAc in hexane as eluent. The desired product was eluted at 10% EtOAc in hexane and the fractions with pure product were concentrated to obtain product as brown solid 2-chloro-5-((trimethylsilyl)ethynyl)pyridin-4-amine (13 g, 65%). ¹H NMR (DMSO-d₆, 400 MHz): δ 7.959 (s, 1H), 6.639 (s, 1H), 6.534 (brs, 2H), 0.237 (s, 9H).

### Step 3: 6-Chloro-1H-pyrrolo[3,2-c]pyridine

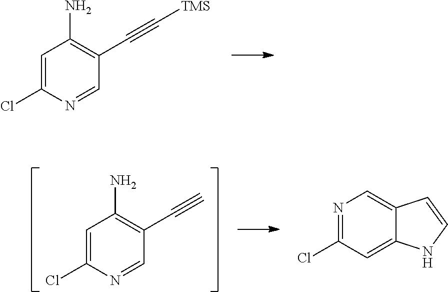

To a cooled solution of 2-chloro-5-((trimethylsilyl)ethynyl)pyridin-4-amine (1 g, 1.0 eq) in DMF (10 mL) under nitrogen atmosphere in a microwave vial (20-30 mL, Biotage), potassium tert-butoxide was added portion wise and stirred for 5 min. The vial was closed and heated at 160° C. in a microwave (Biotage synthesizer) for 50 min. The mixture was poured in water (10 mL) and extracted with EtOAc (2×25 mL). The organic layer was washed with brine (10 mL), dried over anhydrous sodium sulfate and concentrated to obtain the crude. The crude was purified by column chromatography (silica gel, 100-200#) using EtOAc in hexane as eluent. The desired product eluted at 20% EtOAc in hexane and concentration of the pure fractions provided 6-chloro-1H-pyrrolo[3,2-c]pyridine as white solid (0.57 g, 85%). ¹H NMR (DMSO-d₆, 400 MHz): δ 11.626 (s, 1H), 8.621 (s, 1H), 7.491 (d, 2H), 6.606 (s, 1H).

### C. 6-Chloro-1H-pyrazolo[4,3-c]pyridine

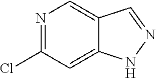

### Step 1: Ethyl 4,6-dihydroxynicotinate

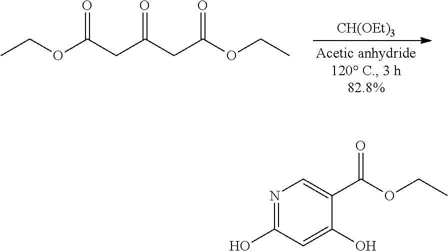

Diethyl 1,3-acetonedicarboxylate (2×50 g, 1.0 eq, Aldrich) was added in portions to acetic anhydride (2×58.6 g, 2.0 eq) in a round bottomed flask. To this solution, triethyl orthoformate (2×51 g, 1.2 eq) was added slowly and the contents heated under a reflux condenser at 120° C. for 3 h. The contents were cooled to 0° C. and 25% aqueous ammonia was added slowly while stirring. The mixture continued stirring for 1 h at 0° C. and the mixture acidified with 3N HCl solution. The solid that separated out was filtered and dried under vacuum to obtain product as white solid ethyl 4,6-dihydroxynicotinate (75.1 g, 82.8%). LCMS: Purity: 99.84%, MS: 184.18 (M+H).

### Step 2: Ethyl 4,6-dichloronicotinate

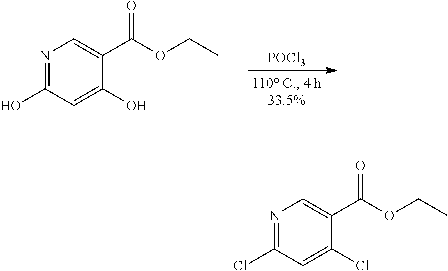

To ethyl 4,6-dihydroxynicotinate (75 g, 1.0 eq) in a round bottomed flask at 0° C., phosphorus oxychloride (500 mL) was added drop wise with stirring and the contents heated at 110° C. for 4 h while monitoring by TLC. After TLC showed completion of starting material, the excess phosphorus oxychloride was removed under vacuum and the residue quenched with ice cold water (50 mL) at 0° C. The mixture was extracted with EtOAc (3×500 mL) and combined organic extract was washed with saturated NaHCO₃ solution (100 mL), brine (100 mL), dried over anhydrous sodium sulphate, filtered and concentrated. The resulting crude material was purified by column chromatography (silica gel 60-120#) using EtOAc in hexane as eluent. The desired product was eluted using 5% EtOAc in hexane and the fractions with pure product were concentrated to obtain ethyl 4,6-dichloronicotinate as off-white solid (41.9 g, 57%). ¹H NMR (400 MHz, CDCl₃): δ 8.845 (s, 1H), 7.475 (s, 1H), 4.466 (q, 2H), 1.424 (t, 3H).

### Step 3: 4,6-Dichloronicotinaldehyde from ester

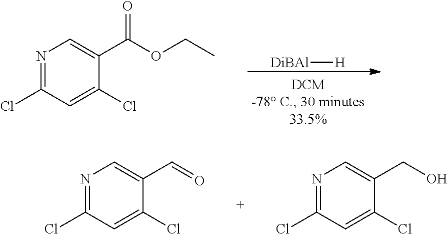

To a stirred solution of ethyl 4,6-dichloronicotinate (36.8 g, 1.0 eq) in DCM (250 mL) at −78° C. under nitrogen atmosphere, DiBAL-H (1.2 eq, 1M solution in toluene) was added drop wise and stirred at −78° C. for 30 min while monitoring by TLC. After TLC showed completion of starting material, the solution was quenched with saturated ammonium chloride solution (50 mL) at −78° C. and diluted with DCM (1 L). The layers were separated and the organic layer was washed with brine (200 mL), dried over anhydrous sodium sulphate, filtered and concentrated. The resulting crude material was purified by flash chromatography (Combiflash®-Redisep, 120 g) using EtOAc in hexane as the eluent. The 4,6-dichloronicotinaldehyde was eluted at 7% EtOAc in hexane and the (4,6-dichloropyridin-3-yl)methanol was eluted at 10% EtOAc in hexane. The aldehyde fractions were concentrated to obtain aldehyde as white solid (9.86 g, 33.5%). ¹H NMR (400 MHz, CDCl₃): δ 10.428 (s, 1H), 8.837 (s, 1H), 7.489 (s, 1H). The alcohol fractions were concentrated to obtain alcohol as off-white solid (4.314 g). ¹H NMR (400 MHz, DMSO-d₆): δ 8.455 (s, 1H), 7.761 (s, 1H), 5.601 (d, 1H), 4.594 (d, 2H).

### Step 3a: 4,6-Dichloronicotinaldehyde from Alcohol

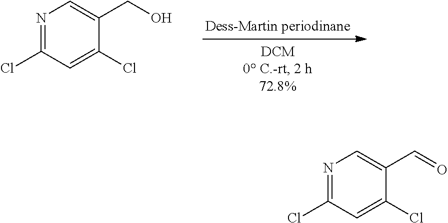 [SMILES: OCc1cnc(Cl)cc1Cl ; "Dess-Martin periodinane" ; O=Cc1cnc(Cl)cc1Cl]

To a solution of (4,6-dichloropyridin-3-yl)methanol (4.9 g, 1.0 eq) in DCM (150 mL) at 0° C. under nitrogen atmosphere, Dess-Martin periodinane (17.5 g, 1.5 eq) was added and stirred at rt for 2 h while monitoring by TLC. After TLC showed completion of starting material, the solution was quenched with water (20 mL) and filtered through Celite® bed. The filtrate was extracted with DCM (150 mL) and the organic layer was washed with saturated NaHCO₃ solution (100 mL), brine (100 mL), dried over anhydrous sodium sulphate, filtered and concentrated. The resulting crude was purified by flash chromatography (Combiflash®-Redisep, 120 g) using EtOAc in hexane as the eluent. The 4,6-dichloronicotinaldehyde was eluted at 7% EtOAc in hexane. The aldehyde fractions were concentrated to obtain aldehyde as off-white solid (3.5 g, 72.76%). ¹H NMR (400 MHz, CDCl₃): δ 10.432 (s, 1H), 8.842 (s, 1H), 7.489 (s, 1H).

### Step 4: 6-Chloro-1H-pyrazolo[4,3-c]pyridine

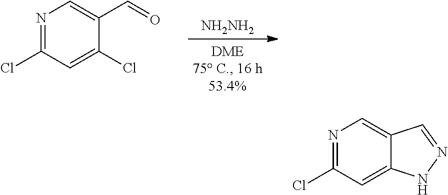

To a stirred solution of 4,6-dichloronicotinaldehyde (3×1.0 g, 1.0 eq) in DME (3×14 mL), hydrazine hydrate (3×1.14 g, 4.0 eq, 99%) was added slowly in a vial. The vial was sealed and the contents heated at 75° C. for 16 h. After TLC showed completion, the mixture was cooled to rt and diluted with water (3×10 mL) and EtOAc (3×10 mL). After combining all 3 mixtures, the layers were separated and the organic layer was washed with brine (20 mL), dried over anhydrous sodium sulphate, filtered and concentrated. The resulting crude was purified by flash chromatography (Combiflash®-Redisep, 12 g) using MeOH in DCM as eluent. The desired product was eluted at 1% MeOH in DCM. The fractions with product were concentrated to obtain pure 6-chloro-1H-pyrazolo[4,3-c]pyridine as yellow solid (1.4 g, 53.43%, from 3 batches). ¹H NMR (400 MHz, DMSO-d₆): δ 13.608 (s, 1H), 8.946 (s, 1H), 8.350 (s, 1H), 7.652 (s, 1H). LCMS calculated for (M) 423.15 and found (M+H) 424.23.

### D. N-(3-((2-chloro-7H-pyrrolo[2,3-d]pyrimidin-7-yl)methyl)pyridin-2-yl)-N-methylmethanesulfonamide

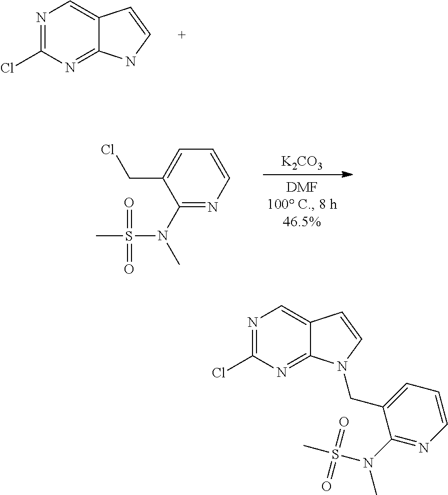

The mixture of 2-chloro-7H-pyrrolo[2,3-d]pyrimidine (3.0 g, 1.0 eq, Bepharm), N-(3-(chloromethyl)pyridin-2-yl)-N-methylmethanesulfonamide (4.6 g, 1.0 eq), K₂CO₃ (8.11 g, 3.0 eq) in DMF (30.0 mL) was heated at 100° C. for 8 h while monitoring by TLC. After completion of starting material, cooled to rt and poured into ice cold water (50 mL). The mixture was extracted with EtOAc (2×150 mL) and the combined organic layer was washed with brine (20 mL), dried over anhydrous sodium sulphate, filtered and concentrated. The resulting crude residue was purified by flash chromatography using EtOAc in hexane as eluent. The desired was eluted at 40% EtOAc in hexane and the fractions with product were concentrated to obtain yellow solid N-(3-((2-chloro-7H-pyrrolo[2,3-d]pyrimidin-7-yl)methyl)pyridin-2-yl)-N-methylmethanesulfonamide (3.2 g, 46.5%). ¹H NMR (400 MHz, CDCl₃): δ 8.957 (s, 1H), 8.479 (t, 1H), 7.70 (d, 1H), 7.38 (m, 2H), 6.756 (d, 1H), 5.577 (s, 2H), 3.215 (s, 3H), 3.148 (s, 3H). MS calculated for (M+H) 352.06 found 352.08.

### E. N-(2-((2-chloro-7H-pyrrolo[2,3-d]pyrimidin-7-yl)methyl)phenyl)-N-methylmethanesulfonamide

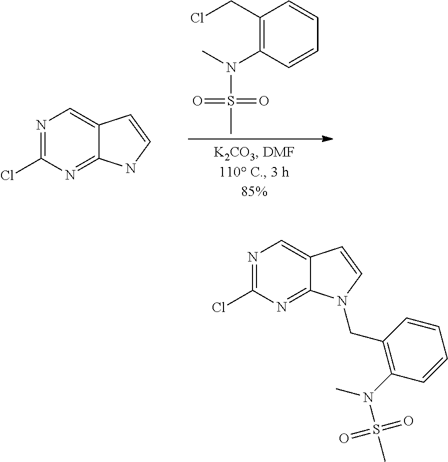 [SMILES: Clc1ncc2cc[nH]c2n1 ; CN(c1ccccc1CCl)S(C)(=O)=O ; CN(c1ccccc1Cn1ccc2cnc(Cl)nc21)S(C)(=O)=O]

To the solution of 2-chloro-7H-pyrrolo[2,3-d]pyrimidine (4.0 g, 1 eq) and N-(2-(chloroethyl)phenyl)-N-methylmethanesulfonamide (6.39 g, 1.05 eq) in DMF (40 mL), K₂CO₃ (10.82 g, 3.0 eq) was added and the mixture heated at 110° C. for 3 h while monitoring by TLC. After completion, the mixture was poured in ice water and extracted with EtOAc (3×100 mL) and the organic layer was concentrated. The resulting residue was washed with ether (5 mL) to obtain N-(2-((2-chloro-7H-pyrrolo[2,3-d]pyrimidin-7-yl)methyl)phenyl)-N-methylmethanesulfonamide as off-white solid (7.8 g, 85%). ¹H NMR (400 MHz, DMSO-d₆): δ 8.95 (s, 1H), 7.95 (s, 1H), 7.68 (m, 1H), 7.59 (m, 1H), 7.30 (m, 1H), 6.91 (m, 1H), 6.75 (m, 1H), 5.71 (d, 1H), 5.43 (d, 1H), 3.40 (s, 3H), 3.08 (s, 3H). MS calculated for (M) 350.06 and found (M+H) 350.99.

### F. N-(3-((6-chloro-1H-pyrrolo[3,2-c]pyridin-1-yl)methyl)pyridin-2-yl)-N-methylmethanesulfonamide

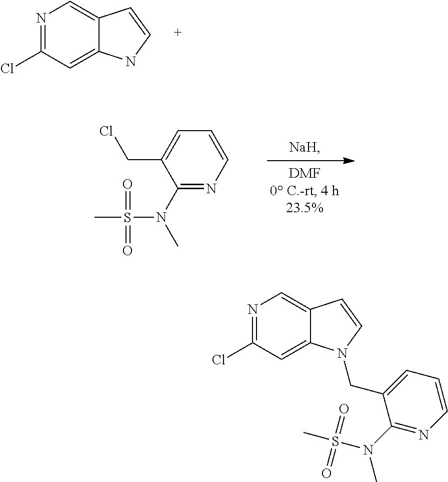 [SMILES: Clc1cc2[nH]ccc2cn1 ; CN(c1ncccc1CCl)S(C)(=O)=O ; CN(c1ncccc1Cn1ccc2cnc(Cl)cc21)S(C)(=O)=O]

The mixture of 6-chloro-1H-pyrrolo[3,2-c]pyridine (0.650 g, 1.0 eq), in DMF (15.0 mL) under nitrogen atmosphere at 0° C., sodium hydride (0.31 g, 60% in mineral oil, 3.0 eq) was added portion wise and stirred for 20 min. N-(3-(chloromethyl)pyridin-2-yl)-N-methylmethanesulfonamide (1.5 g, 1.5 eq) was added slowly and stirred at rt for 4 h while monitoring by TLC. After completion of starting material, quenched the reaction with saturated ammonium chloride solution (20 mL), extracted with EtOAc (3×80 mL) and the combined organic layer was washed with brine (20 mL), dried over anhydrous sodium sulphate, filtered and concentrated. The resulting crude residue was purified by flash chromatography using EtOAc in hexane as eluent. The desired was eluted at 50% EtOAc in hexane and the fractions with product were concentrated to obtain yellow solid N-(3-((6-chloro-1H-pyrrolo[3,2-c]pyridin-1-yl)methyl)pyridin-2-yl)-N-methylmethanesulfonamide (0.35 g, 23.5%). ¹H NMR (400 MHz, DMSO-d₆): δ 8.667 (s, 1H), 8.482 (t, 1H), 7.58 (d, 1H), 7.488 (s, 1H), 7.384 (m, 1H), 7.22 (d, 1H), 6.746 (d, 1H), 5.593 (s, 2H), 3.183 (s, 3H), 3.118 (s, 3H). MS calculated for (MO) 350.06 found (M+H) 351.27.

### G. 2-((2-Chloro-7H-pyrrolo[2,3-d]pyrimidin-7-yl)sulfonyl)benzonitrile

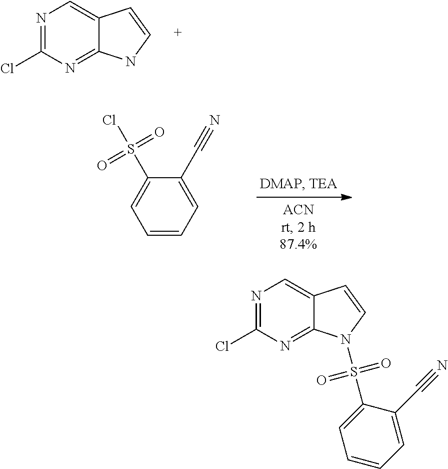 [SMILES: Clc1ncc2cc[nH]c2n1 ; N#Cc1ccccc1S(=O)(=O)Cl ; N#Cc1ccccc1S(=O)(=O)n1ccc2cnc(Cl)nc21]

To a stirred solution of 2-chloro-7H-pyrrolo[2,3-d]pyrimidine (0.50 g, 1.0 eq) in ACN (10 mL), DMAP (0.2 eq) and TEA (3.0 eq) were added under nitrogen atmosphere. To this solution, 2-cyanobenzene sulfonyl chloride (1.2 eq) was added slowly and stirred at rt for 2 h while monitoring by TLC. After completion of starting material, the mixture was diluted with water (20 mL) and extracted with EtOAc (2×100 mL). The organic layer was washed with brine (20 mL), dried over anhydrous sodium sulphate, filtered and concentrated. The resulting crude residue was purified by flash chromatography using MeOH in DCM as eluent. The desired product was eluted at 0.5% MeOH in DCM and the fractions with product were concentrated to obtain off-white solid 2-((2-chloro-7H-pyrrolo[2,3-d]pyrimidin-7-yl)sulfonyl)benzonitrile (0.90 g, 87.4%). ¹H NMR (CDCl₃, 400 MHz): δ 8.827 (s, 1H), 8.723 (d, 1H), 7.990 (d, 1H), 7.936 (t, 1H), 7.788-7.849 (m, 2H), 6.741 (d, 1H). LCMS calculated for (M) 318.00 and found (M+H) 319.03. LCMS showed 99.71% purity.

### H. 8-((6-Chloro-1H-pyrazolo[3,4-d]pyrimidin-1-yl)sulfonyl)quinoline

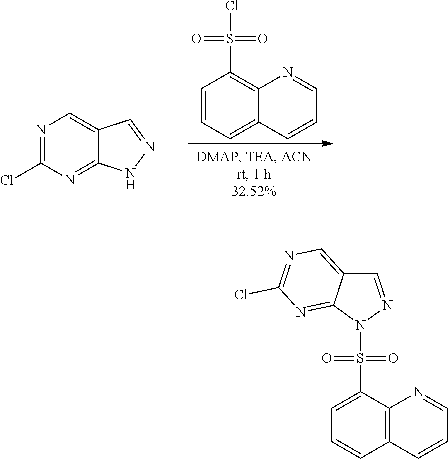 [SMILES: Clc1ncc2cn[nH]c2n1 ; O=S(=O)(Cl)c1cccc2cccnc12 ; O=S(=O)(c1cccc2cccnc12)n1ncc2cnc(Cl)nc21]

A stirred mixture of 6-chloro-1H-pyrazolo[3,4-d]pyrimidine (1.1 g, 0.007 mol, 1.0 eq), quinoline-8-sulfonyl chloride (1.99 g, 1.2 eq), TEA (3.0 eq), DMAP (0.35 eq) in ACN (20 mL) in a round bottomed flask was taken under N₂ atmosphere at rt for 1 h while monitoring by TLC. After completion of starting material, the mixture was poured in water, extracted with ethyl acetate (2×100 mL), the organic layer dried over Na₂SO₄ and the solvent distilled off to get the crude material. The crude material was purified through Combiflash® chromatography using MeOH in DCM as eluent, and the desired compound eluted at 1.2% MeOH in DCM. The isolated product was distilled off under reduced pressure to obtain white solid 8-((6-chloro-1H-pyrazolo[3,4-d]pyrimidin-1-yl)sulfonyl)quinoline (0.80 g, 32.52%). ¹H NMR (400 MHz, DMSO-d₆): δ 9.364 (s, 1H), 8.631-8.612 (d, 1H), 8.584 (s, 1H), 8.525-8.504 (d, 1H), 8.474-8.438 (m, 2H), 7.904-7.865 (m, 1H), 7.574-7.543 (m, 1H). LCMS calculated for (M) 345.20 and found (M+H) 346.01.

### Synthesis of Intermediates

### A. N-(3-(chloromethyl)pyridin-2-yl)-N-methylmethanesulfonamide

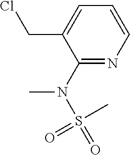

### Step 1: N-methylmethanesulfonamide

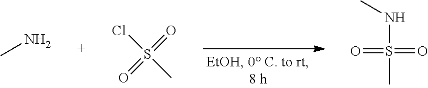

To a stirred solution of methylamine in EtOH (8M in EtOH, 525 mL, 4.8 eq) at 0° C., methane sulfonyl chloride (100 g, 1.0 eq) was added drop wise and stirred at rt for 16 h. After completion, the solvent was distilled off under vacuum and the residue was diluted with DCM (200 mL). The solid that separated out was filtered off and filtrate was concentrated to obtain product N-methylmethanesulfonamide as orange coloured oil (82 g, %). ¹H NMR (CDCl₃, 400 MHz): δ 4.819 (brs, 1H), 2.929 (s, 3H), 2.789 (s, 3H).

### Step 2: N-(3-cyanopyridin-2-yl)-N-methylmethanesulfonamide

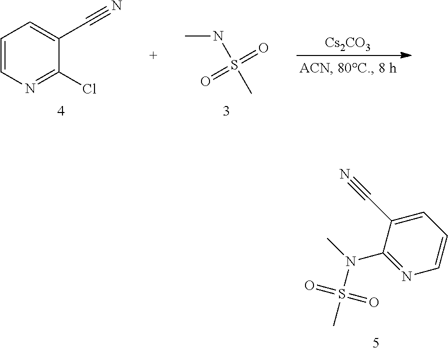

To a stirred solution of 2-chloronicotinonitrile (Aldrich, 100.0 g, 1.0 eq) in ACN (1.0 L) was added cesium carbonate (704 g, 3.0 eq) followed by N-methylmethanesulfonamide (118 g, 1.5 eq). The contents were heated at 80° C. for 8 h while monitoring by TLC. After completion, the solution was cooled to rt and filtered. The filtrate was concentrated and the resulting residue was diluted with DCM (1.0 L) and water (500 mL). The layers were separated and the organic layer was washed with brine (250 mL), dried over anhydrous sodium sulphate, filtered and concentrated to obtain the crude product. The crude product was triturated with mixture of diethyl ether (150 mL), DCM (10 mL) and methanol (10 mL) and filtered to obtain N-(3-cyanopyridin-2-yl)-N-methylmethanesulfonamide as a brown solid (82 g, 53.8%).

### Step 3: N-(3-formylpyridin-2-yl)-N-methylmethanesulfonamide

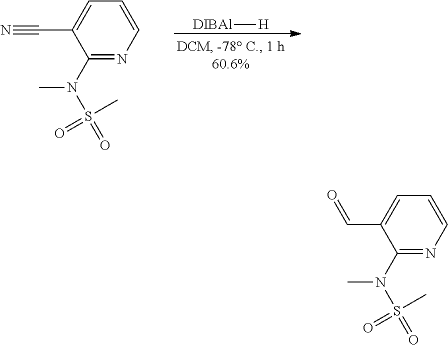

To a stirred solution of N-(3-cyanopyridin-2-yl)-N-methylmethanesulfonamide (60.0 g, 1.0 eq) in DCM (300 mL) at −78° C. under nitrogen atmosphere, DiBAL-H (4.0 eq, 1.0M solution in toluene) was added slowly and stirred at −78° C. for 1 h while monitoring by TLC. After completion, the mixture was quenched with 3N HCl (100 mL) at −78° C. and warmed to rt. The layers were separated and the aqueous layer was further extracted with DCM (2×100 mL). The combined organic extract was washed with brine (100 mL), dried over anhydrous sodium sulphate, filtered and concentrated to obtain the crude product. The crude product was triturated with pentane (50 mL) and filtered to obtain N-(3-formylpyridin-2-yl)-N-methylmethanesulfonamide as a brown solid (36.9 g, 60.6%). ¹H NMR (DMSO-d₆, 400 MHz): δ 10.154 (s, 1H), 8.789 (t, 1H), 8.229 (m, 1H), 7.623 (m, 1H), 3.366 (s, 3H), 3.063 (s, 3H).

### Step 4: N-(3-(hydroxymethyl)pyridin-2-yl)-N-methylmethanesulfonamide

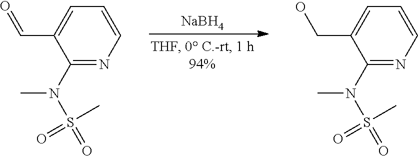

To a stirred solution of N-(3-formylpyridin-2-yl)-N-methylmethanesulfonamide (36.0 g, 1.0 eq) in THF (360 mL) at 0° C., sodium borohydride (18.6 g, 3.0 eq) was added slowly and the mixture stirred at rt for 1 h while monitoring by TLC. After completion, the mixture was quenched with cold water (100 mL) and the layers separated. The aqueous layer was further extracted with EtOAc (2×100 mL) and the combined organic extract was dried over anhydrous sodium sulphate, filtered and concentrated to obtain the crude product. The crude product was triturated with pentane (50 mL) and filtered to obtain N-(3-(hydroxymethyl)pyridin-2-yl)-N-methylmethanesulfonamide as light brown solid (34 g, 94.4%). ¹H NMR (DMSO-d₆, 400 MHz): δ 8.384 (t, 1H), 7.964 (d, 1H), 7.450 (dd, 1H), 4.603 (s, 2H), 3.834 (s, 1H), 3.097 (s, 3H), 3.075 (s, 3H).

### Step 5: N-(3-(chloromethyl)pyridin-2-yl)-N-methylmethanesulfonamide

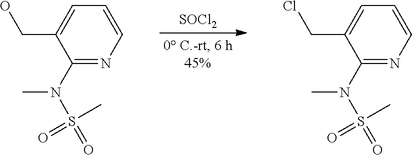

To a stirred solution of N-(3-(hydroxymethyl)pyridin-2-yl)-N-methylmethanesulfonamide (34.0 g, 1.0 eq) in DCM (340 mL) at 0° C., thionyl chloride (56.1 g, 3.0 eq) was added drop wise and stirred at rt for 6 h while monitoring by TLC. After completion, the excess thionyl chloride was removed under vacuum. The resulting residue was diluted with EtOAc (500 mL) and washed with saturated NaHCO₃ solution (2×400 mL). The organic layer was dried over anhydrous sodium sulphate, filtered and concentrated to obtain the crude product. The crude product was purified by Combiflash® flash chromatography (80 g, silica gel) using EtOAc in hexane as eluent. The product was eluted at 60% EtOAc in hexane. The fractions with product were concentrated to obtain N-(3-(chloromethyl)pyridin-2-yl)-N-methylmethanesulfonamide as yellow solid (16.2 g, 45%). ¹H NMR (DMSO-d₆, 400 MHz): δ 8.534 (t, 1H), 8.086 (d, 1H), 7.517 (dd, 1H), 4.865 (s, 2H), 3.218 (s, 3H), 3.185 (s, 3H).

### B. N-(2-(Chloroethyl)phenyl)-N-methylmethanesulfonamide

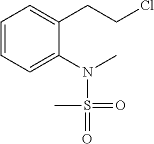

### Step 1: Methyl 2-(methylsulfonamido)benzoate

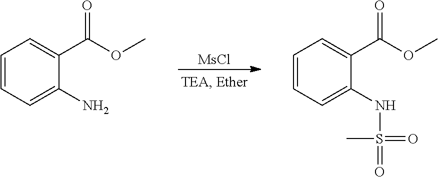

To a stirred solution of methyl 2-aminobenzoate (25 g, 1.0 eq) and triethylamine (23 ml, 1.0 eq) in ether (200 mL) at 0° C., methane sulfonyl chloride (13 mL, 1.0 eq) was added drop wise and the mixture stirred at rt for 3 h while monitoring by TLC. After completion, the reaction was quenched with ice cold water and extracted with EtOAc (3×200 mL). The organic layer was washed with water (50 mL), brine (50 mL), dried over anhydrous sodium sulfate and concentrated. The resulting crude product was purified by column chromatography (silica gel, 100-200#) using EtOAc in hexane as the eluent. The product eluted at 20% EtOAc in hexane. The fractions with product were concentrated to obtain methyl 2-(methylsulfonamido)benzoate as white solid (37 g, 97.5%). ¹H NMR (400 MHz, CDCl₃): δ 10.459 (s. 1H), 8.062 (d, 1H), 7.745 (d, 1H), 7.579 (t, 1H), 7.129 (t, 1H), 3.941 (s, 3H), 3.063 (s, 1H). LCMS calculated for (M) 229.04 found (M+H) 230.04, (M−H) 227.95. Purity: 96.47%.

### Step 2: Methyl 2-(N-methylmethylsulfonamido)benzoate

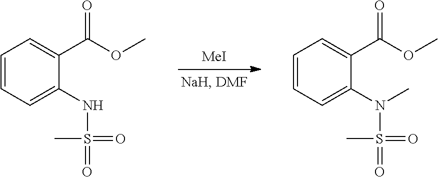 [SMILES: "MeI" ; COC(=O)c1ccccc1NS(C)(=O)=O ; COC(=O)c1ccccc1N(C)S(C)(=O)=O]

To a suspension of NaH (1.7 eq) in DMF (150 mL), methyl 2-(methylsulfonamido)benzoate (37 g, 1.0 eq) was added slowly and stirred for 1 h. To this mixture, excess methyl iodide (98.5 ml) was added and the mixture stirred at rt for 16 h. After TLC showed completion, the mixture was quenched with saturated ammonium chloride solution (100 mL). The aqueous layer was extracted with EtOAc (3×300 ml) and the combined organic layer was washed with water (100 mL), brine (50 mL), dried over anhydrous sodium sulfate and concentrated to obtain methyl 2-(N-methylmethylsulfonamido)benzoate as white solid (28 g, 71.4%). ¹H NMR (400 MHz, CDCl₃): δ 7.908 (dd, 1H), 7.559 (t. 1H), 7.461-7.394 (m, 2H), 3.928 (s, 3H), 3.312 (s, 1H), 2.970 (s, 3H). LCMS calculated for (M) 243.06 found (M+H) 244.0. Purity: 97.52%.

### Step 3: N-(2-(hydroxymethyl)phenyl)-N-methylmethanesulfonamide

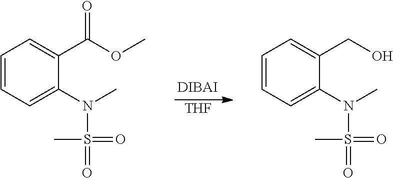 [SMILES: COC(=O)c1ccccc1N(C)S(C)(=O)=O ; CN(c1ccccc1CO)S(C)(=O)=O ; "THF"]

To a stirred solution of methyl 2-(N-methylmethylsulfonamido)benzoate (27.5 g, 1.0 eq) in DCM (270 ml) at 0° C., 1M DiBAL-H solution in toluene (3.0 eq) was added drop wise and stirred at rt for 3 h while monitoring by TLC. After completion, the reaction was quenched with saturated ammonium chloride solution (100 mL) and acidified with 3N HCl. The aqueous layer was extracted with EtOAc (3×200 mL) and the combined organic layer was washed with water (100 mL), brine (50 mL), dried over anhydrous sodium sulfate and concentrated to obtain N-(2-(hydroxymethyl)phenyl)-N-methylmethanesulfonamide as white solid (18 g, 73.9%). ¹H NMR (400 MHz, CDCl₃): δ 7.613 (d, 1H), 7.434-7.360 (m, 2H), 7.271-7.260 (t. 1H), 4.830 (dd, 2H), 3.289 (s, 3H), 3.312 (s, 1H), 2.985 (s, 3H).

### Step 4: N-(2-(chloroethyl)phenyl)-N-methylmethanesulfonamide

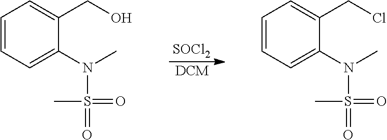

To a stirred solution of N-(2-(hydroxymethyl)phenyl)-N-methylmethanesulfonamide (18.0 g, 1.0 eq) in DCM (180 mL) at 0° C., SOCl₂ (3.0 eq) was added drop wise and stirred from 0° C. to 80° C. for 3 h while monitoring by TLC. After completion, the reaction was quenched with ice cold water (100 mL). The aqueous layer was extracted with DCM (3×200 mL) and the combined organic layer was washed with water (100 mL), dried over anhydrous sodium sulfate and concentrated to obtain N-(2-(chloroethyl) phenyl)-N-methylmethanesulfonamide as white solid (16 g, 82.1%). ¹H NMR (400 MHz, CDCl₃): δ 7.576 (t, 1H), 7.395 (m, 2H), 7.229 (m, 1H), 5.056-4.575 (dd, 2H), 3.318 (s, 3H), 2.977 (s, 3H). LCMS calculated for (M) 233.72 found (M+H) 234.11. Purity: 97.86%.

### C. 2-(difluoromethoxy)benzene-1-sulfonyl chloride

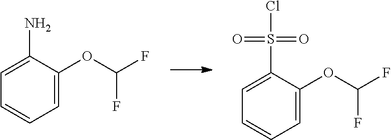 [SMILES: Nc1ccccc1OC(F)F ; O=S(=O)(Cl)c1ccccc1OC(F)F]

To a solution of 2-(difluoromethoxy)aniline (5.0 g) in glacial acetic acid (15 mL) and acetonitrile (75 mL) at rt, concentrated hydrochloric acid (10 mL) in water (15 mL) was slowly added while stirring. After 10 min, a solution of sodium nitrite (2.16 g, 1.0 eq) in water (10 mL) was added at 5-10° C. and the mixture was stirred at rt for 2 h to obtain the diazonium salt. In a separate flask, a saturated solution of sulfur dioxide in glacial acetic acid (50 mL) was prepared at rt and a solution of copper (II) chloride (4.21 g, 1.0 eq) in water (15 mL) was added. The mixture of the diazonium salt which had been prepared beforehand was then added to the solution of the copper salt at 0° C. The resulting mixture was stirred at rt for an additional 6 h. The mixture was then poured into ice-cooled water. The aqueous layer was extracted with EtOAc (3×100 mL). The combined organic extract was washed with saturated sodium bicarbonate solution (50 mL), dried over sodium sulfate, and concentrated under reduced pressure to obtain 2-(difluoromethoxy)benzene-1-sulfonyl chloride as colorless liquid (4.7 g, 55%). ¹H NMR (CDCl₃, 400 MHz): δ 8.076 (dd, 1H), 7.763 (t, 1H), 7.470 (d, 1H), 7.428 (t, 1H), 6.873-6.512 (t, 1H).

Preparation of SO₂ in AcOH: SO₂ gas was passed into AcOH present in a round bottomed flask (with outlet) at 0° C. for 30 min.

### D. 4-Methylquinoline-8-sulfonyl chloride

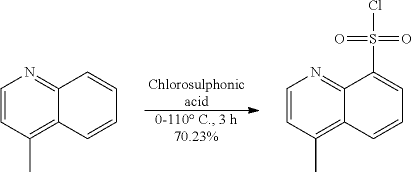

4-Methyl quinoline (1.0 g, 0.00699 mol, 1.0 eq) was added drop wise to chlorosulphonic acid (10 mL, 10 vol) in a dry round bottomed flask at 0° C. and the mixture heated at 110° C. for 3 h while monitoring by TLC. After completion of starting material, the mixture was poured in ice cold water and the aqueous layer was extracted with ethyl acetate (3×100 mL). The organic layer was washed with water (20 mL), brine (20 mL), dried over anhydrous sodium sulfate and concentrated to obtain crude product. The resulting crude was purified by Combiflash® column chromatography with 20% EtOAc in hexane as eluting solvent to obtain compound 4-methylquinoline-8-sulfonyl chloride (1.18 g, 70.23%) as white solid.

### E. 6-Amino-3,4-dihydroquinolin-2(1H)-one

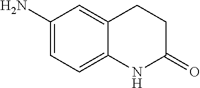 [SMILES: Nc1ccc2c(c1)CCC(=O)N2]

### Step 1: 3-Chloro-N-phenylpropanamide

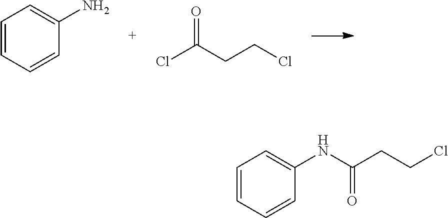 [SMILES: Nc1ccccc1 ; O=C(Cl)CCCl ; O=C(CCCl)Nc1ccccc1]

To a solution of aniline (10 g) in water: acetone (2:1; 300 mL) in a round bottomed flask at 0° C., potassium carbonate (1.5 eq) was added and the mixture stirred for 10 min. To this mixture, 3-chloropropanoyl chloride (1.26 eq) was added drop wise and stirred at 0° C. for 2 h while monitoring by TLC. After completion, the mixture was diluted with ice cold water, filtered and the solid washed with water and dried to obtain 3-chloro-N-phenylpropanamide as white solid (18.5 g, 94%). ¹H NMR (CDCl₃, 400 MHz): δ 7.528-7.508 (d, 2H), 7.402 (brs, 1H), 7.349-7.310 (t, 2H), 7.148-7.112 (t, 1H), 3.88 (t, 2H), 2.816 (t, 2H).

### Step 2: 3,4-Dihydroquinolin-2(1H)-one

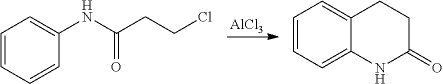

To the solution of 3-chloro-N-phenylpropanamide (17 g) in chlorobenzene (425 mL) under nitrogen atmosphere at 0° C., aluminum chloride (74 g, 6 eq) was added portion wise and the mixture stirred at 120° C. for 12 h. After TLC showed completion, the mixture was cooled to rt and quenched slowly with ice cold water. The mixture was extracted with DCM (2×200 mL) and the organic layer was dried over anhydrous sodium sulfate, filtered and concentrated to a residue. The resulting crude reside was purified by column chromatography over silica gel using EtOAc in hexane as eluent. The product eluted at 25-30% EtOAc in hexane. The fractions with pure product were concentrated to obtain 3,4-dihydroquinolin-2(1H)-one as red solid (5.5 g, 40%). ¹H NMR (CDCl₃, 400 MHz): δ 7.986 (brs, 1H), 7.194-7.157 (2H), 6.992 (t, 1H), 6.76 (d, 1H), 2.975 (t, 2H), 2.644 (t, 2H).

### Step 3: 6-Nitro-3,4-dihydroquinolin-2(1H)-one

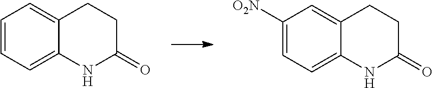 [SMILES: O=C1CCc2ccccc2N1 ; O=C1CCc2cc([N+](=O)[O-])ccc2N1]

To a solution of 3,4-dihydroquinolin-2(1H)-one (1 g) in H₂SO₄ (20 mL) at −10° C., water (5 mL) was added dropwise with stirring. To this solution, concentrated HNO₃ (0.5 mL) was added dropwise with stirring, while cooling to a temperature of 0° C. The resulting solution was stirred for 15 min at −10° C. After completion, the mixture was quenched by adding ice water (50 mL). The resulting solution was extracted with EtOAc (5×50 mL). The combined organic layers were concentrated under reduced pressure and the resulting crude product was purified by column chromatography over silica gel using EtOAc in hexane as eluent. The product eluted at 50-70% EtOAc in hexane. The fractions with pure product were concentrated to obtain 6-nitro-3,4-dihydroquinolin-2(1H)-one as light brown solid (0.9 g, 69%). ¹H NMR (CDCl₃, 400 MHz): δ 8.549 (brs 1H), 8.116 (brs, 2H), 6.879 (t, 1H), 3.095 (t, 2H), 2.720 (t, 2H).

### Step 4: 6-Amino-3,4-dihydroquinolin-2(1H)-one

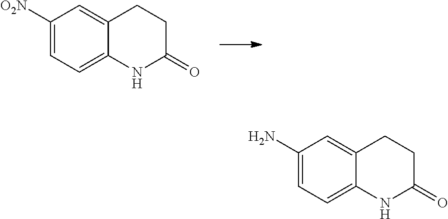 [SMILES: O=C1CCc2cc([N+](=O)[O-])ccc2N1 ; Nc1ccc2c(c1)CCC(=O)N2]

To a solution of 6-nitro-3,4-dihydroquinolin-2(1H)-one (3 g) in MeOH (60 mL) at 0° C., Zn dust (5 eq) and ammonium chloride (5 eq) were added portion wise and the mixture stirred at rt for 1 h while monitoring by TLC. After completion, the mixture was filtered over Celite® bed and resulting filtrate concentrated. The residue was diluted with 5% MeOH in DCM and washed with water. The organic layer was dried over sodium sulfate, filtered and concentrated to obtain 6-amino-3,4-dihydroquinolin-2(1H)-one (2.3 g, 91%). ¹H NMR (DMSO-d₆, 400 MHz): δ 9.654 (brs, 1H), 6.53 (d, 1H), 6.378-6.334 (m, 2H), 4.707 (brs, 2H), 2.699 (t, 2H), 2.330 (t, 2H).

### F. (S)-tert-butyl 4-(4-aminophenyl)-2-methylpiperazine-1-carboxylate

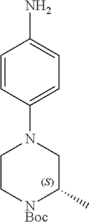

### Step 1: (S)-tert-butyl 2-methyl-4-(4-nitrophenyl)piperazine-1-carboxylate

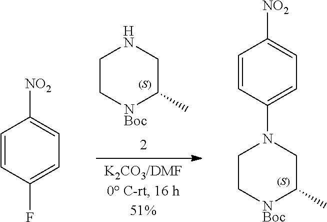

To a suspension of 1-fluoro-4-nitrobenzene (3.5 g, 24.8 mmol) and K₂CO₃ (10.2 g, 74.4 mmol) in DMF (15 mL) at 0° C., (S)-tert-butyl 2-methylpiperazine-1-carboxylate (4.9 g, 24.8 mmol) was added and the mixture was stirred at rt overnight. The mixture was diluted with ice cold water (50 mL), the resulting precipitate was filtered and solid was washed with hexane (30 mL) to obtain (S)-tert-butyl 2-methyl-4-(4-nitrophenyl)piperazine-1-carboxylate (4.0 g, 51% yield). ¹H NMR (400 MHz, DMSO-d₆): δ 8.03 (d, 2H), 6.94 (d, 2H), 4.15-4.13 (m, 1H), 3.84-3.73 (m, 3H), 3.36-3.05 (m, 3H), 1.40 (s, 9H), 1.08 (d, 3H). LCMS m/z calcd for [M+H]⁺ 322.37. found 222.3.

### Step 2: (S)-tert-butyl 4-(4-aminophenyl)-2-methylpiperazine-1-carboxylate

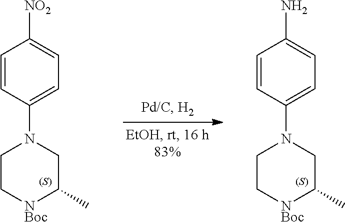

A solution of (S)-tert-butyl 2-methyl-4-(4-nitrophenyl)piperazine-1-carboxylate (4.0 g, 12.40 mmol) in MeOH (20 mL) was purged with argon, Pd/C (0.4 g, 10%) added, the mixture was charged with a H₂ bladder and the mixture was stirred at rt overnight. The mixture was filtered through a Celite® bed and washed with MeOH (100 mL). The filtrate was evaporated under reduced pressure to obtain (S)-tert-butyl 4-(4-aminophenyl)-2-methylpiperazine-1-carboxylate (3.0 g, 83%). ¹H NMR (400 MHz, DMSO-d₆): δ 6.65 (d, 2H), 6.47 (d, 2H), 6.04-6.02 (m, 1H), 4.57 (s, 2H), 4.10 (s, 1H), 3.73 (d, 1H), 3.16 (d, 1H), 3.09-3.04 (m, 2H), 2.57-2.53 (m, 1H), 2.41-2.35 (m, 1H), 1.39 (s, 9H), 1.22 (d, 3H). LCMS m/z calcd for [M+H]⁺ 292.39. found 291.3.

### G. (R)-tert-butyl 4-(4-aminophenyl)-2-methylpiperazine-1-carboxylate

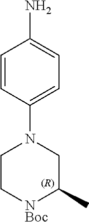

### Step 1: (R)-tert-butyl 2-methyl-4-(4-nitrophenyl)piperazine-1-carboxylate

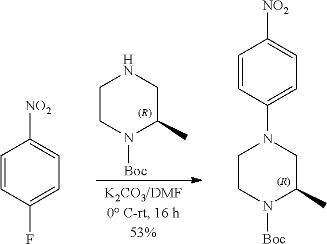

To a suspension of 1-fluoro-4-nitrobenzene (3.5 g, 24.8 mmol) and K₂CO₃ (10.2 g, 74.4 mmol) in DMF (15 mL) at 0° C., (R)-tert-butyl 2-methylpiperazine-1-carboxylate (4.9 g, 24.8 mmol) was added and the mixture was stirred at rt overnight. The mixture was diluted with ice cold water (50 mL) and the resulting precipitate was filtered and the solid was washed with hexane (30 mL) to obtain (R)-tert-butyl 2-methyl-4-(4-nitrophenyl)piperazine-1-carboxylate (4.2 g, 53% yield). ¹H NMR (400 MHz, DMSO-d₆): δ 8.03 (d, 2H), 6.94 (d, 2H), 4.15-4.13 (m, 1H), 3.84-3.74 (m, 3H), 3.36-3.05 (m, 3H), 1.40 (s, 9H), 1.08 (d, 3H), LCMS m/z calcd for [M+H]⁺ 322.37. found 222.2.

### Step 2: (R)-tert-butyl 4-(4-aminophenyl)-2-methylpiperazine-1-carboxylate

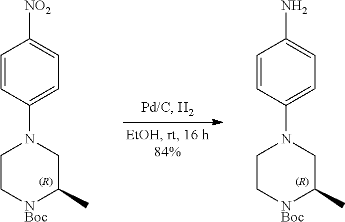

A solution of (R)-tert-butyl 2-methyl-4-(4-nitrophenyl)piperazine-1-carboxylate (4.2 g, 13.0 mmol) in MeOH (20 mL) was purged with argon, Pd/C (0.42 g, 10%) was added, the mixture was charged with a H₂ bladder and the mixture was stirred at rt overnight. The mixture was filtered through a Celite® bed and washed with MeOH (100 mL). The filtrate was evaporated under reduced pressure to obtain (R)-tert-butyl 4-(4-aminophenyl)-2-methylpiperazine-1-carboxylate (3.2 g, 84% yield). ¹H NMR (400 MHz, DMSO-d₆): δ 6.66 (d, 2H), 6.47 (d, 2H), 6.04-6.02 (m, 1H), 4.56 (s, 2H), 4.14 (s, 1H), 3.74 (d, 1H), 3.16 (d, 1H), 3.09-3.04 (m, 2H), 2.57-2.54 (m, 1H), 2.41-2.35 (m, 1H), 1.39 (s, 9H), 1.22 (d, 3H). LCMS m/z calcd for [M+H]⁺ 292.39. found 291.3.

### H. 3-Methyl-4-morpholinoaniline

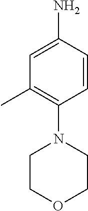

### Step 1: 4-(2-Methyl-4-nitrophenyl)morpholine

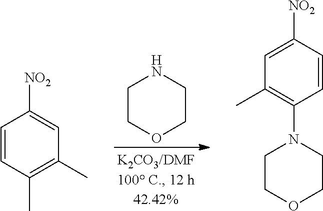 [SMILES: Cc1ccc([N+](=O)[O-])cc1C ; C1COCCN1 ; Cc1cc([N+](=O)[O-])ccc1N1CCOCC1]

A stirred mixture of 1-fluoro-2-methyl-4-nitrobenzene (10 g, 0.0645 mol, 1.0 eq), morpholine (8.4 g, 0.096 mol, 1.5 eq), K₂CO₃ (26.7 g, 0.1935 mol, 3 eq), DMF (50 mL) in a round bottomed flask was heated at 100° C. for 3 h while monitoring by TLC. After completion of the starting material, the reaction mass was cooled to rt, diluted with cold water (500 mL) and extracted with ethyl acetate (2×100 mL). The organic layer was separated, washed with brine solution, dried over Na₂SO₄ and concentrated under reduced pressure to obtain crude product. The crude product was purified by Combiflash® chromatography using EtOAc in hexane as eluent. The product was eluted at 20% EtOAc in hexane. The fractions with pure product were concentrated to obtain pale yellow 4-(2-methyl-4-nitrophenyl)morpholine (6.1 g, 42.42%). ¹H NMR (400 MHz, CDCl₃): δ 8.06 (s, 1H), 8.06 (d, 1H), 7.01 (d, 1H), 3.886 (t, 4H), 3.033 (t, 4H), 2.377 (s, 3H).

### Step 2: 3-Methyl-4-morpholinoaniline

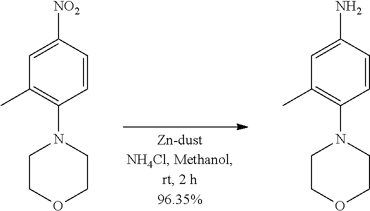 [SMILES: Cc1cc([N+](=O)[O-])ccc1N1CCOCC1 ; Cc1cc(N)ccc1N1CCOCC1]

To a stirred mixture of 4-(2-methyl-4-nitrophenyl)morpholine (6 g, 0.0645 mol, 1.0 eq), Zn dust (8.64 g, 0.135 mol, 5 eq) in methanol (50 mL) in a round bottomed flask at 0° C., ammonium chloride (7.29 g, 0.135 mol, 5 eq) was added slowly and the mixture stirred at rt for 1 h while monitoring by TLC. After completion of the starting material, the reaction mass was filtered through a Celite® bed and filtrate was concentrated under reduced pressure to obtain a residue. The residue was diluted with DCM (150 mL) and washed with water. The organic layer was separated, washed with brine sol, dried over sodium sulphate and concentrated under reduced pressure to obtain 3-methyl-4-morpholinoaniline as brown solid (5 g, 96.35%). ¹H NMR (400 MHz, CDCl₃): δ 6.768 (d, 1H), 6.39 (s, 1H), 6.36 (d, 1H), 4.66 (brs, 2H), 3.681 (t, 4H), 2.683 (t, 4H), 2.124 (s, 3H).

### I. 2-(4-Aminophenyl)hexahydropyrrolo[1,2-a]pyrazin-6(2H)-one

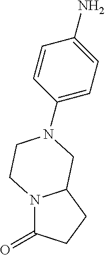 [SMILES: Nc1ccc(N2CCN3C(=O)CCC3C2)cc1]

### Step 1: tert-Butyl 3-(pyrazin-2-yl)propanoate

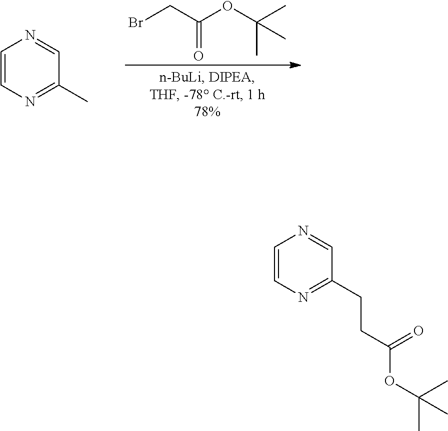

To a stirred solution of N,N-diisopropylethylamine (6.4 g, 0.06382 mol, 1.2 eq) in THF (100 mL) in a round bottomed flask under nitrogen atmosphere at −78° C., n-BuLi in THF solution (0.06382 mol, 1.2 eq) was added drop wise and the mixture stirred at rt for 1 h. The mixture was cooled to −78° C. and 2-methylpyrazine (5 g, 0.05319 mol, 1.0 eq) was added drop wise. After 30 min stirring at −78° C., tert-butyl 2-bromoacetate (10.31 g, 0.05319 mol, 1.0 eq) was added drop wise and stirred for 1 h at −78° C. while monitoring by TLC. After completion of the starting material, the reaction was quenched with saturated ammonium chloride solution at −78° C. and the temperature slowly raised to rt. The mixture was extracted with ethyl acetate (3×150 mL) and the organic layer was washed with water (50 mL), brine (50 mL), dried over anhydrous sodium sulphate and concentrated to obtain product tert-butyl 3-(pyrazin-2-yl)propanoate (4.2 g, 78.35%) as pale brown liquid.

### Step 2: tert-Butyl 3-(piperazin-2-yl)propanoate

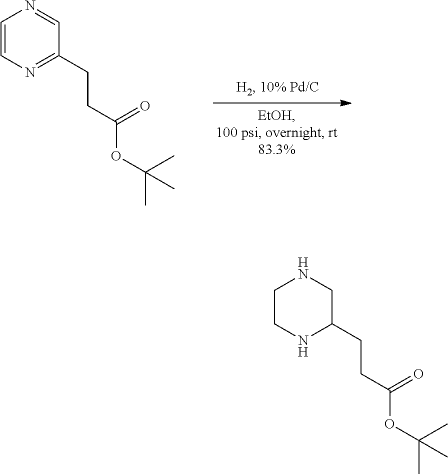

To a solution of tert-butyl 3-(pyrazin-2-yl)propionate (4.1 g, 0.01971 mol, 1.0 eq) in ethanol (100 mL) in a clean and dry hydrogenating steel vessel, 10% palladium on carbon (3 g, 50% wet) was added, under nitrogen atmosphere. The mixture was hydrogenated at 100 psi for overnight. After TLC showed completion of starting material, the mixture was filtered through a Celite® bed and the filtrate was concentrated to obtain tert-butyl 3-(piperazin-2-yl)propanoate (3.5 g, 83.3%) as pale brown liquid.

### Step 3: Hexahydropyrrolo[1,2-a]pyrazin-6(2H)-one

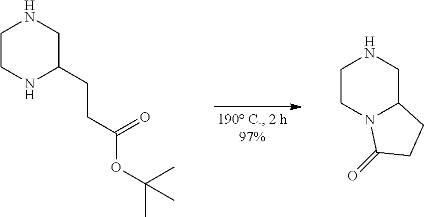

Tert-butyl 3-(piperazin-2-yl)propanoate (3.4 g, 0.01585 mol, 1.0 eq) in a round bottomed flask was heated at 190° C. for 2 h while monitoring by TLC. After completion of starting material, the mixture was cooled and washed with hexane to obtain hexahydropyrrolo[1,2-a]pyrazin-6(2H)-one (2.2 g, 97%) as pale brown solid.

### Step 4: 2-(4-Nitrophenyl)hexahydropyrrolo[1,2-a]pyrazin-6(2H)-one

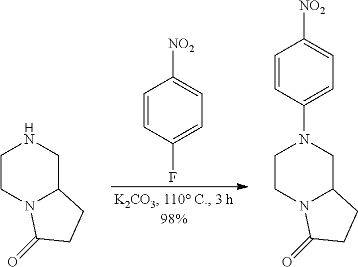 [SMILES: O=C1CCC2CNCCN12 ; O=[N+]([O-])c1ccc(F)cc1 ; O=C1CCC2CN(c3ccc([N+](=O)[O-])cc3)CCN12]

To a solution of hexahydropyrrolo[1,2-a]pyrazin-6(2H)-one (2.2 g, 0.01571 mol, 1.0 eq) in DMF (20 mL) in a vial, 1-fluoro-4-nitrobenzene (2.2 g, 0.01571 mol, 1.0 eq) and K₂CO₃ (6.5 g, 0.04714 mol, 3.0 eq) were added. The vial was sealed and the mixture heated at 110° C. for 3 h while monitoring by TLC. After completion of the starting material, the mixture was poured into ice water (50 mL) and extracted with DCM (3×150 mL). The organic layer was washed with water (50 mL), brine (50 mL), dried over anhydrous sodium sulphate and concentrated. The resulting residue was purified by column chromatography on silica gel (100-200#) using methanol in DCM as the eluent. The product eluted at 4% methanol in DCM and the concentration of the fractions afforded 2-(4-nitrophenyl)hexahydropyrrolo[1,2-a]pyrazin-6(2H)-one (4.02 g, 98%) as pale yellow solid.

### Step 5: 2-(4-Aminophenyl)hexahydropyrrolo[1,2-a]pyrazin-6(2H)-one

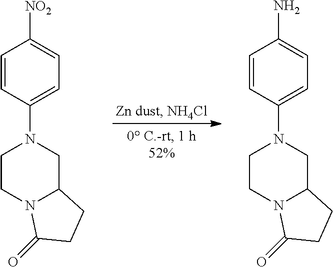 [SMILES: O=C1CCC2CN(c3ccc([N+](=O)[O-])cc3)CCN12 ; "Zn dust, NH4Cl" ; Nc1ccc(N2CCN3C(=O)CCC3C2)cc1]

To a mixture of 2-(4-nitrophenyl)hexahydropyrrolo[1,2-a]pyrazin-6(2H)-one (4.01 g, 0.01532 mol, 1.0 eq) and Zn dust (4.9 g, 0.076628 mol, 5.0 eq) in methanol (120 mL) at 0° C., NH₄Cl (4.1 g, 0.076628 mol, 5.0 eq) was added portion wise and stirred at rt for 1 h while monitoring by TLC. After completion of starting material, the mixture was filtered through a Celite® bed and the bed washed with methanol (20 mL). The filtrate was concentrated, the resulting residue was suspended in DCM (100 mL) and washed with water (2×20 mL). The organic layer was dried over Na₂SO₄ and concentrated to obtain 2-(4-aminophenyl)hexahydropyrrolo[1,2-a]pyrazin-6(2H)-one (1.82 g, 52%) as brown solid. ¹H NMR (400 MHz, DMSO-d₆): δ 6.715 (d, 2H), 6.485 (s, 1H), 8.471 (d, 2H), 4.640 (s, 2H), 3.835 (d, 1H), 3.660 (m, 1H), 3.435 (d, 1H), 3.28 (d, 2H), 2.873 (d, 1H), 2.258 (m, 5H), 1.575 (m, 1H).

### J. tert-Butyl 4-(4-amino-3-chlorophenyl)piperazine-1-carboxylate

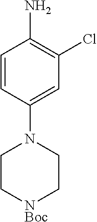 [SMILES: CC(C)(C)OC(=O)N1CCN(c2ccc(N)c(Cl)c2)CC1]

### Step 1: tert-Butyl 4-(3-chloro-4-nitrophenyl)piperazine-1-carboxylate

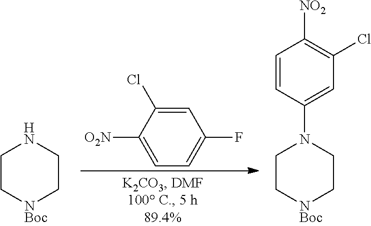

A stirred mixture of 2-chloro-4-fluoro-1-nitrobenzene (2.0 g, 1.0 eq), tert-butyl piperazine-1-carboxylate (2.33 g, 1.1 eq), K₂CO₃ (4.73 g, 3.0 eq) in DMF (20 mL) in a two necked round bottomed flask was heated at 100° C. for 5 h while monitoring by TLC. After completion of starting material, the mixture was quenched with water and extracted with EtOAc (3×100 mL). The organic layer was dried over Na₂SO₄, filtered and concentrated to obtain tert-butyl 4-(3-chloro-4-nitrophenyl)piperazine-1-carboxylate as yellow solid (3.4 g, 89.4%). ¹H NMR (400 MHz, CDCl₃): δ 8.025 (dd, 1H), 6.838 (d, 1H), 6.709 (dd, 1H), 3.594 (t, 4H), 3.388 (t, 4H), 1.484 (s, 9H).

### Step 2: tert-Butyl 4-(4-amino-3-chlorophenyl)piperazine-1-carboxylate

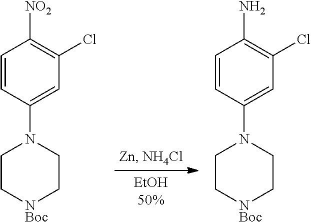 [SMILES: CC(C)(C)OC(=O)N1CCN(c2ccc([N+](=O)[O-])c(Cl)c2)CC1 ; CC(C)(C)OC(=O)N1CCN(c2ccc(N)c(Cl)c2)CC1]

A stirred solution of tert-butyl 4-(3-chloro-4-nitrophenyl) piperazine-1-carboxylate (3.3 g, 1.0 eq) in EtOH (40 mL) in a two necked round bottomed flask was cooled to 0° C., and Zn dust (3.14 g, 5.0 eq), followed by NH₄Cl (2.58 g, 5.0 eq), were added and the contents were stirred at rt for 3 h while monitoring by TLC. After completion of starting material, the mixture was filtered through a Celite® bed and the filtrate concentrated to obtain the crude material. This crude material was diluted with water and extracted with DCM. The organic layer was dried over Na₂SO₄, filtered and concentrated to obtain tert-butyl 4-(4-amino-3-chlorophenyl)piperazine-1-carboxylate as bluish solid (1.5 g, 50%). ¹H NMR (400 MHz, CDCl₃): δ 6.803 (d, 1H), 6.737 (m, 2H), 4.818 (d, 2H), 3.411 (brs, 4H), 2.864 (t, 4H), 1.409 (s, 9H).

### K. (1-(4-Aminophenyl)piperidin-3-yl)methanol

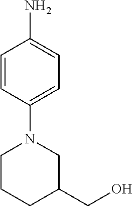 [SMILES: Nc1ccc(N2CCCC(CO)C2)cc1]

### Step 1: (1-(4-Nitrophenyl)piperidin-3-yl)methanol

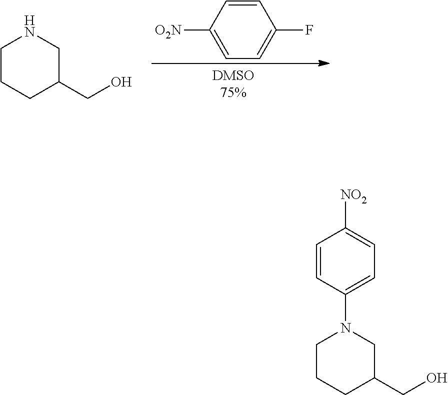 [SMILES: OCC1CCCNC1 ; O=[N+]([O-])c1ccc(F)cc1 ; O=[N+]([O-])c1ccc(N2CCCC(CO)C2)cc1]

A stirred mixture of piperidin-3-ylmethanol (2.0 g, 1.0 eq), 1-fluoro-4-nitrobenzene (2.94 g, 1.2 eq), in DMSO (20 mL) in a vial was closed and the contents heated at 100° C. for 12 h while monitoring by TLC. After completion of starting material, the mixture was quenched with water and extracted with EtOAc. The organic layer was dried over Na₂SO₄, filtered and concentrated to obtain (1-(4-nitrophenyl)piperidin-3-yl)methanol as yellow solid (3.0 g, 75%). ¹H NMR (400 MHz, DMSO-d₆): δ 8.08 (d, 1H), 6.805 (d, 1H), 3.98 (m, 1H), 3.81 (d, 1H), 3.64 (m, 1H), 3.53 (m, 1H), 3.041 (m, 1H), 2.878 (m, 1H), 1.838 (m, 3H), 1.621 (m, 2H), 1.294 (d, 1H).

### Step 2: (1-(4-Aminophenyl)piperidin-3-yl)methanol

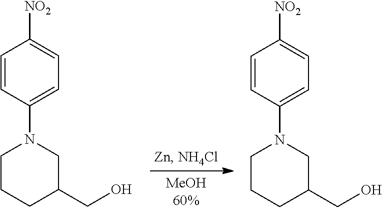 [SMILES: O=[N+]([O-])c1ccc(N2CCCC(CO)C2)cc1 ; O=[N+]([O-])c1ccc(N2CCCC(CO)C2)cc1]

A stirred mixture of (1-(4-nitrophenyl)piperidin-3-yl)methanol (2.95 g, 1.0 eq), in EtOH (40 mL) in a two necked round bottomed flask was cooled to 0° C., then Zn (4.06 g, 5.0 eq), followed by ammonium chloride (3.375 g, 5.0 eq), were added and the contents stirred at rt for 3 h while monitoring by TLC. After completion of starting material, the mixture was filtered through a Celite® bed and the filtrate concentrated to obtain the crude product. This crude product was diluted with water and extracted with DCM. The organic layer was dried over Na₂SO₄, filtered and concentrated to obtain (1-(4-aminophenyl)piperidin-3-yl)methanol as bluish solid (1.5 g, 60%) LCMS showed 92% purity.

### L. 4-Thiomorpholinoaniline

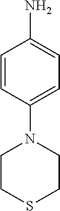 [SMILES: Nc1ccc(N2CCSCC2)cc1]

### Step 1: 4-(4-Nitrophenyl)thiomorpholine

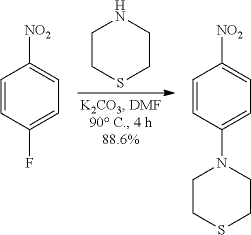

To a stirred mixture of 4-fluoronitrobenzene (5.0 g, 1.0 eq) and thiomorpholine (3.65 g, 1.0 eq) in DMF (50 mL) in a vial, K₂CO₃ (14.6 g, 3.0 eq) was added and the mixture was heated to 90° C. for 4 h while monitoring by TLC. After completion of the reaction, the mixture was cooled to rt, poured into ice water and extracted with EtOAc (3×100 ml). The organic layer was washed with brine, cold water, dried over anhydrous sodium sulphate, and concentrated to obtain crude a yellow solid which was washed with n-hexane to obtain 4-(4-nitrophenyl)thiomorpholine as yellow solid (7.0 g, 88.6%). ¹H NMR (400 MHz, DMSO-d₆): δ 8.060 (d, 2H), 7.025 (d, 2H), 3.886 (t, 4H), 2.645 (t, 4H), LCMS calculated for (M) 224.06 found (M+H) 224.97.

### Step 2: 4-Thiomorpholinoaniline

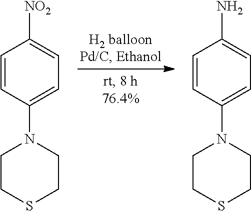 [SMILES: O=[N+]([O-])c1ccc(N2CCSCC2)cc1 ; Nc1ccc(N2CCSCC2)cc1]

To a solution of 4-(4-nitrophenyl)thiomorpholine (2.0 g, 1.0 eq) in ethanol (20 mL) in a round bottomed flask under nitrogen atmosphere at rt, 10% Pd/C (50% wet, 2.0 g) was added slowly and the mixture was hydrogenated using a hydrogen balloon at rt for 8 h. After TLC showed completion of reaction, the mixture was filtered through a Celite® bed and washed with 10% methanol in DCM (20 mL). The filtrate was collected and distilled off the solvent at rotavapour to get 4-thiomorpholinoaniline as bluish solid (1.30 g, 76.4%). ¹H NMR (400 MHz, DMSO-d₆): δ 6.680 (d, 2H), 6.470 (d, 2H) 4.634 (s, 2H), 3.167 (t, 4H), 2.675 (t, 4H), LCMS calculated for (M) 194.30 and found (M+H) 195.03.

### M. tert-Butyl 4-(4-amino-2-(hydroxymethyl)phenyl)piperazine-1-carboxylate

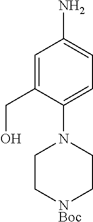 [SMILES: CC(C)(C)OC(=O)N1CCN(c2ccc(N)cc2CO)CC1]

### Step 1: tert-Butyl 4-(2-formyl-4-nitrophenyl)piperazine-1-carboxylate

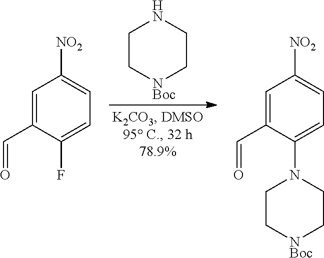

To a stirred mixture of 2-fluoro-5-nitrobenzaldehyde (1.0 g, 1.0 eq) and tert-butyl piperazine-1-carboxylate (1.20 g, 1.1 eq) in DMSO (10 mL) in a vial, K₂CO₃ (2.4 g, 3.0 eq) was added and the mixture was heated to 95° C. for 32 h. After TLC showed completion of reaction, the mixture was cooled to rt, poured in to ice water and extracted with EtOAc (3×25 mL). The organic layer was washed with brine, cold water, dried over anhydrous sodium sulphate, and concentrated to obtain crude yellow solid which was washed with n-hexane to obtain tert-butyl 4-(2-formyl-4-nitrophenyl)piperazine-1-carboxylatas yellow solid (1.5 g, 78.9%). ¹H NMR (400 MHz, DMSO-d₆): δ 10.045 (s, 1H), 8.529 (d, 1H), 8.315 (m, 1H), 7.294 (d, 1H), 3.536 (m, 4H), 3.324 (m, 2H), 2.539 (m, 2H), 1.394 (s, 9H). LCMS calculated for (M) 335.15 found (M+H) 336.15.

### Step 2: tert-Butyl 4-(2-(hydroxymethyl)-4-nitrophenyl)piperazine-1-carboxylate

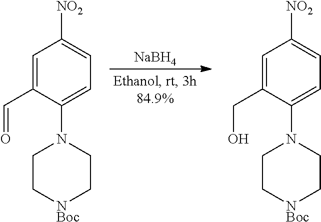 [SMILES: CC(C)(C)OC(=O)N1CCN(c2ccc([N+](=O)[O-])cc2C=O)CC1 ; "NaBH4" ; CC(C)(C)OC(=O)N1CCN(c2ccc([N+](=O)[O-])cc2CO)CC1]

To a mixture of tert-butyl 4-(2-formyl-4-nitrophenyl)piperazine-1-carboxylate (0.70 g, 1.0 eq) in ethanol (10 mL) in a vial, sodium borohydride (2 eq) was added slowly and stirred at rt for 3 h while monitoring by TLC. After completion, the solvent was distilled off, diluted with water (10 mL) and extracted with EtOAc (2×15 mL). The organic layer was dried over anhydrous sodium sulfate and concentrated. The resulting solid was washed with ether (4 mL) to obtain tert-butyl 4-(2-(hydroxymethyl)-4-nitrophenyl)piperazine-1-carboxylate as yellow solid (0.60 g, 84.9%). ¹H NMR (400 MHz, DMSO-d₆): δ 8.332 (s, 1H), 8.094 (m, 1H), 7.160 (d, 1H), 5.554 (t, 1H), 5.547 (d, 2H), 3.477 (m, 4H), 2.957 (m, 4H), 1.40 (s, 9H), LCMS calculated for (M) 338.16 and found (M+H) 337.99.

### Step 3: tert-Butyl 4-(4-amino-2-(hydroxymethyl)phenyl)piperazine-1-carboxylate

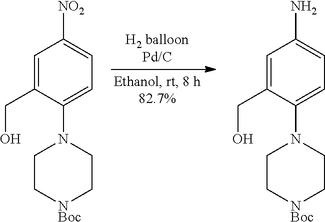 [SMILES: CC(C)(C)OC(=O)N1CCN(c2ccc([N+](=O)[O-])cc2CO)CC1 ; CC(C)(C)OC(=O)N1CCN(c2ccc(N)cc2CO)CC1]

To the solution of tert-butyl 4-(2-(hydroxymethyl)-4-nitrophenyl)piperazine-1-carboxylate (0.60 g, 1.0 eq) in ethanol (10 mL) in a round bottomed flask under nitrogen atmosphere at rt, 10% Pd/C (50% wet, 2.0 g) was added slowly and the mixture was hydrogenated using a hydrogen balloon at rt for 8 h. After TLC showed completion of the reaction, the mixture was filtered through a Celite® bed. The filtrate was concentrated on rotavapour to provide tert-butyl 4-(4-amino-2-(hydroxymethyl)phenyl)piperazine-1-carboxylate as bluish gummy solid (0.45 g, 82.7%). LCMS calculated for (M) 308.19 and found (M+H) 308.17.

### N. 1-(4-Aminophenyl)-N,N-dimethylpyrrolidin-3-amine

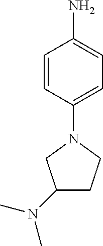

### Step 1: N,N-dimethyl-1-(4-nitrophenyl)pyrrolidin-3-amine

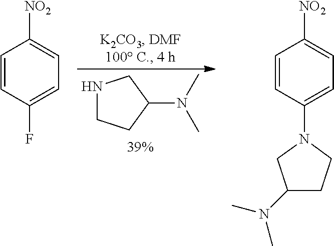

To a solution of N,N-dimethylpyrrolidin-3-amine (0.70 g, 1.2 eq) in DMF (12 mL) under nitrogen atmosphere at rt in a vial, 4-fluoronitrobenzene (0.785 g, 1.0 eq) and K₂CO₃ (2.1 g, 3.0 eq) were added, the vial sealed and heated at 100° C. for 4 h. After TLC showed completion of the reaction, the mixture was cooled to rt, poured into water (10 mL) and extracted with ethyl acetate (3×20 mL). The organic layer was washed with brine, dried over anhydrous sodium sulfate, filtered and concentrated. The crude material was purified in Combiflash® chromatography using ethyl acetate in hexane as eluent. The product was eluted at 21% ethyl acetate in hexane. Fractions with product were distilled off under reduced pressure in rotavapor to obtain yellow solid N,N-dimethyl-1-(4-nitrophenyl)pyrrolidin-3-amine (0.47 g, 39%). ¹H NMR (400 MHz, CDCl₃): δ 8.134-8.111 (m, 2H), 6.479-6.455 (m, 2H), 3.579-3.552 (m, 2H), 3.447-3.422 (m, 2H), 3.405-3.380 (m, 1H), 3.262-3.217 (m, 1H), 2.954 (t, 2H), 2.3.24 (s, 6H) 2.020-1.917 (m, 1H). LCMS calculated for (M) 235.15 and found (M+H) 236.05.

### Step 2: 1-(4-Aminophenyl)-N,N-dimethylpyrrolidin-3-amine

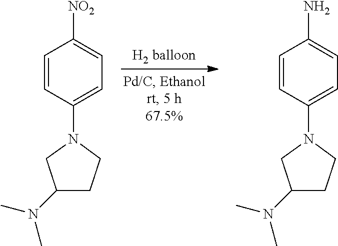 [SMILES: CN(C)C1CCN(c2ccc([N+](=O)[O-])cc2)C1 ; CN(C)C1CCN(c2ccc(N)cc2)C1]

To a solution of N,N-dimethyl-1-(4-nitrophenyl)pyrrolidin-3-amine (0.46 g, 1.0 eq) in ethanol (10 mL) in a round bottomed flask under nitrogen atmosphere at rt, 10% Pd/C (50% wet, 0.5 g) was added slowly and the mixture was hydrogenated using a hydrogen balloon at rt for 4 h. After TLC showed completion of the reaction, the mixture was filtered through a Celite® bed and washed with ethanol (20 mL). The filtrate was collected, the solvent removed under vacuum and the resulting solid was washed with pentane (4 mL) to obtain 1-(4-aminophenyl)-N,N-dimethylpyrrolidin-3-amine (0.27 g, 67.5%). ¹H NMR (400 MHz, CDCl₃): δ 6.687-6.665 (m, 2H), 6.464-6.443 (m, 2H), 3.438-3.398 (m, 1H), 3.330-3.283 (m, 2H), 3.106-3.064 (m, 1H), 2.871-2.793 (m, 1H), 2.301 (s, 6H), 2.213-2.150 (m, 1H) 1.941-1.841 (m, 1H). LCMS calculated for (M) 205.38 and found (M+H) 206.12.

### O. (S)-tert-butyl 4-(4-amino-2-fluorophenyl)-3-methylpiperazine-1-carboxylate

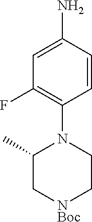 [SMILES: C[C@H]1CN(C(=O)OC(C)(C)C)CCN1c1ccc(N)cc1F]

### Step 1: (S)-tert-butyl 4-(2-fluoro-4-nitrophenyl)-3-methylpiperazine-1-carboxylate

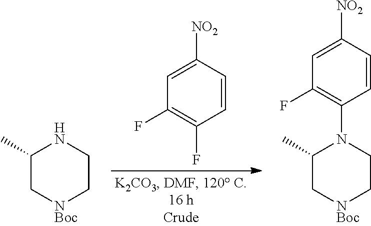 [SMILES: C[C@H]1CN(C(=O)OC(C)(C)C)CCN1 ; O=[N+]([O-])c1ccc(F)c(F)c1 ; C[C@H]1CN(C(=O)OC(C)(C)C)CCN1c1ccc([N+](=O)[O-])cc1F]

A stirred mixture of (S)-tert-butyl 3-methylpiperazine-1-carboxylate (2.0 g, 1.0 eq), 1,2-difluoro-4-nitrobenzene (3.1 g, 2.0 eq) and K₂CO₃ (4.1 g, 3.0 eq) in DMF (25 mL) in a two necked round bottomed flask was heated at 120° C. for 16 h. After completion of the starting material on TLC, the mixture was quenched with ice cold water and extracted with EtOAc (2×250 mL). The organic layer was dried over anhydrous Na₂SO₄, filtered and concentrated to obtain (S)-tert-butyl 4-(2-fluoro-4-nitrophenyl)-3-methylpiperazine-1-carboxylate as yellow solid (4.0 g, crude). ¹H NMR (400 MHz, CDCl₃): δ 7.994-7.966 (dd, 1H), 7.930-7.891 (dd, 1H), 6.911-6.867 (t, 1H), 3.975 (bs, 2H), 3.820-3.789 (d, 1H), 3.361-3.333 (d, 2H), 3.174-3.143 (d, 2H), 1.484 (s, 9H), 1.111-1.094 (d, 3H). LCMS calculated for (M) 339.16 and found (M+H) 340.25. LCMS showed 39% purity.

### Step 2: (S)-tert-butyl 4-(4-amino-2-fluorophenyl)-3-methylpiperazine-1-carboxylate

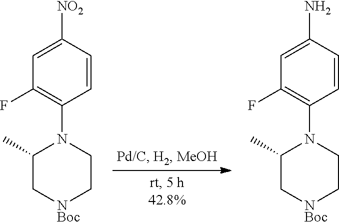 [SMILES: C[C@H]1CN(C(=O)OC(C)(C)C)CCN1c1ccc([N+](=O)[O-])cc1F ; C[C@H]1CN(C(=O)OC(C)(C)C)CCN1c1ccc(N)cc1F]

To a solution of (S)-tert-butyl 4-(2-fluoro-4-nitrophenyl)-3-methylpiperazine-1-carboxylate (4.0 g, 1.0 eq) in methanol (40 mL) in a round bottomed flask under nitrogen atmosphere at rt, 10% Pd/C (50% wet, 4.0 g) was added slowly and the mixture was hydrogenated using a hydrogen balloon at rt for 5 h. After TLC showed completion of the reaction, the mixture was filtered through a Celite® bed and washed with methanol (50 mL). The filtrate was collected and the solvent removed using a rotavapour. The resulting residue was purified by Combiflash® chromatography using EtOAc in hexane as the eluent. The product eluted at 30% EtOAc in hexane. The fractions with product were concentrated to obtain (S)-tert-butyl 4-(4-amino-2-fluorophenyl)-3-methylpiperazine-1-carboxylate as brown gummy liquid (1.5 g, 42.8%). ¹H NMR (400 MHz, CDCl₃): δ 6.900-6.858 (t, 1H), 6.398-6.371 (d, 2H), 3.865 (bs, 1H), 3.773-3.741 (d, 1H), 3.624 (s, 2H), 3.303 (bs, 1H), 3.114 (bs, 1H), 2.963 (bs, 2H), 2.902-2.811 (m, 1H), 1.475 (s, 9H), 0.865-0.849 (d, 3H). LCMS calculated for (M) 309.19 and found (M+H) 310.06. LCMS showed 93.93% purity.

### P. 1-(4-Amino-2-fluorophenyl)-N,N-dimethylpyrrolidin-3-amine

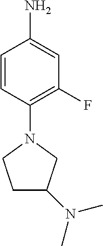 [SMILES: CN(C)C1CCN(c2ccc(N)cc2F)C1]

### Step 1: tert-Butyl 3-(dimethylamino)pyrrolidine-1-carboxylate

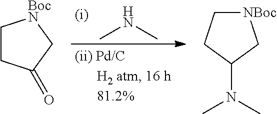

To a stirred solution of tert-butyl 3-oxopyrrolidine-1-carboxylate (1.5 g, 0.0081 mol, 1 eq) in ethanol (30 mL) in a round bottomed flask at 0° C., N,N-dimethylamine (1.2 eq, 2M solution in THF) and acetic acid (2 mL) were added and stirred at rt for 2 h. To this mixture, 10% w/w Pd/C (50% wet, 500 mg) was added and stirred at rt overnight under hydrogen atmosphere. The mixture was filtered through a Celite® bed and washed with methanol (50 mL). The filtrate was concentrated to obtain the tert-butyl 3-(dimethylamino)pyrrolidine-1-carboxylate (5.2 g, 81.2%) as pale brown liquid.

### Step 2: N,N-dimethylpyrrolidin-3-amine hydrochloride

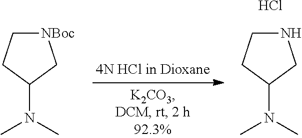

To a stirred solution of tert-butyl 3-(dimethylamino)pyrrolidine-1-carboxylate (5.1 g, 0.2383 mol, 1.0 eq) in DCM (30 ml) in a round bottomed flask at 0° C., 4N HCl in dioxane (10 mL) was added and stirred at rt for 2 h while monitoring by TLC. After completion of starting material, the mixture was concentrated to obtain N,N-dimethylpyrrolidin-3-amine hydrochloride (4.32 g, 92.3%) as off-white solid.

### Step 3: 1-(2-Fluoro-4-nitrophenyl)-N,N-dimethylpyrrolidin-3-amine

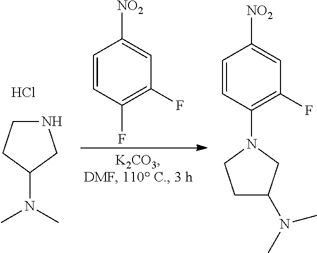

To a stirred solution of 1,2-difluoro-4-nitrobenzene (2 g, 0.01257 mol) in DMF (20 mL) in a round bottomed flask, K₂CO₃ (8.6 g, 5.0 eq), N,N-dimethylpyrrolidin-3-amine (2.3 g, 0.01127 mol. 0.9 eq) were added and the mixture heated at 110° C. for 3 h while monitoring by TLC. After completion of starting material, the mixture was cooled and poured in ice cold water. The aqueous layer was extracted with ethyl acetate (3×100 mL) and the organic layer was washed with water (3×50 mL), dried over sodium sulphate and concentrated to obtain 1-(2-fluoro-4-nitrophenyl)-N,N-dimethylpyrrolidin-3-amine (3.6 g, crude) as pale yellow solid. This material used as such in next step.

### Step 4: 1-(4-Amino-2-fluorophenyl)-N,N-dimethylpyrrolidin-3-amine

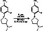 [SMILES: CN(C)C1CCN(c2ccc([N+](=O)[O-])cc2F)C1 ; CN(C)C1CCN(c2ccc(N)cc2F)C1]

To a stirred solution of 1-(2-fluoro-4-nitrophenyl)-N,N-dimethylpyrrolidin-3-amine (3.6 g, 0.016143 mol, 1.0 eq) in ethanol (100 mL) in a round bottomed flask under nitrogen atmosphere, 10% w/w palladium on carbon (50% wet, 0.5 g) was added and the mixture was hydrogenated using a hydrogen balloon for overnight. After TLC showed completion of starting material, the mixture was filtered through a Celite® bed and washed with methanol. The filtrate was concentrated to obtain crude product. The crude material was purified by column chromatography using neutral alumina with methanol in DCM as eluent. The desired product eluted at 10% methanol in DCM and the fractions with product were concentrated to obtain 1-(4-amino-2-fluorophenyl)-N,N-dimethylpyrrolidin-3-amine (0.8 g, 28.5%) as brown oily liquid.

### Q. (S)-tert-butyl 4-(4-amino-2-(hydroxymethyl)phenyl)-3-methylpiperazine-1-carboxylate

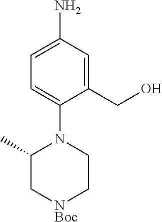 [SMILES: C[C@H]1CN(C(=O)OC(C)(C)C)CCN1c1ccc(N)cc1CO]

### Step 1: (S)-tert-butyl 4-(2-formyl-4-nitrophenyl)-3-methylpiperazine-1-carboxylate

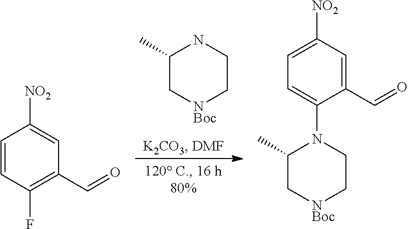 [SMILES: O=Cc1cc([N+](=O)[O-])ccc1F ; C[C@H]1CN(C(=O)OC(C)(C)C)CCN1 ; C[C@H]1CN(C(=O)OC(C)(C)C)CCN1c1ccc([N+](=O)[O-])cc1C=O]

A stirred mixture of (S)-tert-butyl 3-methylpiperazine-1-carboxylate (1.0 g, 1.0 eq), 2-fluoro-5-nitrobenzaldehyde (1.26 g, 1.5 eq) and K₂CO₃ (2.0 g, 3.0 eq) in DMF (15.0 mL) in a two necked round bottomed flask was heated at 120° C. for 16 h. After completion of starting material on TLC, the mixture was quenched with ice cold water and extracted with EtOAc (2×250 mL). The organic layer was dried over anhydrous Na₂SO₄, filtered and concentrated to obtain (S)-tert-butyl 4-(2-formyl-4-nitrophenyl)-3-methylpiperazine-1-carboxylate as yellow liquid (1.4 g, 80%). ¹H NMR (400 MHz, CDCl₃): δ 10.150 (s, 1H), 8.645-8.638 (d, 1H), 8.340-8.310 (dd, 1H), 7.124-7.101 (d, 1H), 4.138-4.077 (m, 1H), 3.810-3.711 (m, 1H), 3.628 (s, 1H), 3.553-3.512 (dd, 1H), 3.418-3.393 (d, 1H), 3.296 (bs, 1H), 3.090-3.061 (d, 1H), 1.455 (s, 9H), 1.091-1.075 (d, 3H). LCMS calculated for (M) 349.38 and found (M+H) 350.00. LCMS showed 98.69% purity.

### Step 2: (S)-tert-butyl 4-(2-(hydroxymethyl)-4-nitrophenyl)-3-methylpiperazine-1-carboxylate

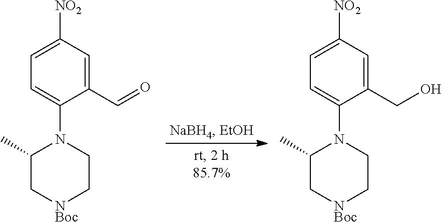 [SMILES: C[C@H]1CN(C(=O)OC(C)(C)C)CCN1c1ccc([N+](=O)[O-])cc1C=O ; C[C@H]1CN(C(=O)OC(C)(C)C)CCN1c1ccc([N+](=O)[O-])cc1CO]

To a solution of (S)-tert-butyl 4-(2-formyl-4-nitrophenyl)-3-methylpiperazine-1-carboxylate (1.4 g, 1.0 eq) in EtOH (15.0 mL) in a round bottomed flask, NaBH₄ (0.304 g, 2.0 eq) was added portionwise at rt and stirred for 2 h. After TLC showed completion, ethanol was removed, the mixture was quenched with water and extracted with EtOAc (2×250 mL). The organic layer was dried over anhydrous Na₂SO₄, filtered and concentrated to obtain (S)-tert-butyl 4-(2-(hydroxymethyl)-4-nitrophenyl)-3-methylpiperazine-1-carboxylate as yellow liquid (1.2 g, 85.7%). ¹H NMR (400 MHz, CDCl₃): δ 8.248-8.254 (d, 1H), 8.160-8.132 (dd, 1H), 7.250-7.228 (d, 1H), 5.295 (s, 1H), 4.963-4.928 (d, 1H), 4.738-4.703 (d, 1H), 3.922-3.886 (d, 1H), 3.817-3.784 (t, 1H), 3.394-3.343 (t, 1H), 3.242-3.200 (m, 1H), 3.070 (bs, 2H), 2.770-2.710 (m, 1H), 1.452 (s, 9H), 0.920-0.905 (d, 3H). LCMS calculated for (M) 351.40 and found (M+H) 352.31. LCMS showed 97.01% purity.

### Step 3: (S)-tert-butyl 4-(4-amino-2-(hydroxymethyl)phenyl)-3-methylpiperazine-1-carboxylate

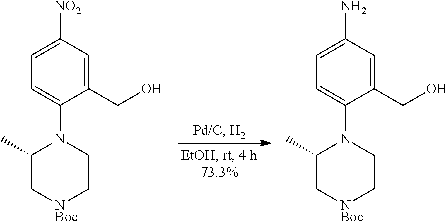 [SMILES: C[C@H]1CN(C(=O)OC(C)(C)C)CCN1c1ccc([N+](=O)[O-])cc1CO ; C[C@H]1CN(C(=O)OC(C)(C)C)CCN1c1ccc(N)cc1CO]

To a solution of (S)-tert-butyl 4-(2-(hydroxymethyl)-4-nitrophenyl)-3-methylpiperazine-1-carboxylate (1.2 g, 1.0 eq) in ethanol (15.0 mL) in a round bottomed flask under nitrogen atmosphere at rt, 10% Pd/C (50% wet, 1.0 g) was added slowly and the mixture was hydrogenated using a hydrogen balloon at rt for 4 h. After TLC showed completion of reaction, the mixture was filtered through a Celite® bed and washed with methanol (50 mL). The filtrate was collected and the solvent removed using a rotavapour to obtain (S)-tert-butyl 4-(4-amino-2-(hydroxymethyl)phenyl)-3-methylpiperazine-1-carboxylate as black solid (1.09 g, 73.3%). ¹H NMR (400 MHz, DMSO-d₆): δ 6.853-6.832 (d, 1H), 6.648-6.642 (d, 1H), 6.413-6.386 (dd, 1H), 4.901-4.846 (m, 3H), 4.554-4.434 (m, 2H), 3.812-3.737 (t, 2H), 3.345 (merged with water peak, 2H), 2.978 (bs, 1H), 2.816-2.787 (t, 1H), 2.728-2.607 (m, 1H), 1.411 (s, 9H), 0.688-0.673 (d, 3H). LCMS calculated for (M) 321.41 and found (M+H) 322.33. LCMS showed 82.22% purity.

### R. tert-Butyl 4-(4-aminophenyl)-2,2-dimethylpiperazine-1-carboxylate

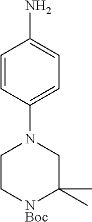 [SMILES: CC(C)(C)OC(=O)N1CCN(c2ccc(N)cc2)CC1(C)C]

### Step 1: 3,3-Dimethylpiperazin-2-one

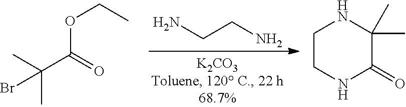

To a solution of ethyl 2-bromo-2-methylpropanoate (20 g, 1.0 eq) in toluene (130 ml), K₂CO₃ (16.6 g, 1.1 eq) was added under nitrogen atmosphere and stirred for 5 min. To this mixture, ethane-1,2-diamine (43 g, 6.5 eq) was added and the mixture heated at 120° C. for 22 h. After TLC showed completion, the mixture was cooled to rt and filtered. The filtrate was concentrated and the residue was washed with ether to obtain 3,3-dimethylpiperazin-2-one as light brown solid (9 g, 68.7%). ¹H NMR (400 MHz, DMSO-d₆): δ 7.376 (brs, 1H), 3.108 (m, 3H), 2.803 (m, 3H), 2.30-2.60 (m, 3H).

### Step 2: 2,2-Dimethylpiperazine

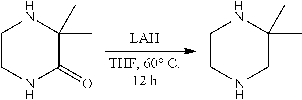

To a solution of 3,3-dimethylpiperazin-2-one (2.0 g, 1.0 eq) in THF (60 mL) under nitrogen atmosphere at 0° C., LAH (2.96 g, 5.0 eq) was added very slowly and portion wise. After stirring at rt for 10 min, the mixture was heated at 65° C. for 12 h. After TLC showed completion of starting material, the mixture was cooled to 0° C. and quenched by adding water (3 mL), 15% NaOH solution (3 mL) and water (8 mL). The contents were stirred for 30 min and filtered. The filtrate was extracted with n-butanol (3×80 mL) and the organic layer was dried over sodium sulfate, concentrated under vacuum to obtain crude 2,2-dimethylpiperazine (1.5 g).

### Step 3: tert-Butyl 2,2-dimethyl-4-(4-nitrophenyl)piperazine-1-carboxylate

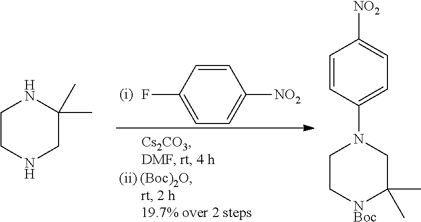

To a solution of 2,2-dimethylpiperazine (1.5 g, crude) in DMF (15 mL) under nitrogen atmosphere at rt, 4-fluoro nitrobenzene (2.25 g, 1.2 eq) and cesium carbonate (8.6 g, 2 eq) were added and stirred at rt for 4 h while monitoring by TLC. After completion of starting material, boc-anhydride (2.85 g, 1 eq) was added and the mixture stirred for 2 h. After TLC showed completion of intermediate, the mixture was poured into water and extracted with ethyl acetate (3×50 mL). The organic layer was dried over sodium sulfate and concentrated to obtain crude product. The crude product was purified using Combiflash® chromatography and desired compound was eluted at 20% EtOAc in hexane. The fractions with product were concentrated to obtain tert-butyl 2,2-dimethyl-4-(4-nitrophenyl)piperazine-1-carboxylate as yellow solid (1 g, 19.7% over 2 steps). ¹H NMR (400 MHz, DMSO-d₆): δ 8.071-8.047 (d, 2H), 6.875-6.851 (d, 2H), 3.805-3.772 (t, 2H), 3.683 (s, 2H), 3.470 (t, 2H) 1.490 (s, 9H), 1.333 (m, 6H).

### Step 4: tert-Butyl 4-(4-aminophenyl)-2,2-dimethylpiperazine-1-carboxylate

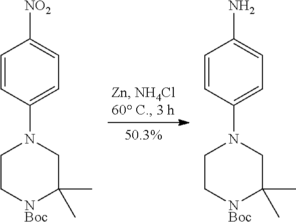 [SMILES: CC(C)(C)OC(=O)N1CCN(c2ccc([N+](=O)[O-])cc2)CC1(C)C ; CC(C)(C)OC(=O)N1CCN(c2ccc(N)cc2)CC1(C)C]

To a solution of tert-butyl 2,2-dimethyl-4-(4-nitrophenyl)piperazine-1-carboxylate (0.60 g, 1.0 eq) in ethanol at rt, zinc dust (0.582 g, 5 eq) and ammonium chloride (0.480 g, 5 eq) were added slowly and stirred this mixture at 60° C. for 3 h while monitoring by TLC. After completion of starting material, the mixture was filtered through a Celite® bed. The filtrate was concentrated, the residue diluted with water (10 mL) and extracted with ethyl acetate (3×80 mL). The organic layer was dried over anhydrous sodium sulfate and concentrated to obtain the crude product. The crude was purified using Combiflash® chromatography using EtOAc in hexane as eluent. The desired product eluted at 31% EtOAc in hexane. The fractions with product were concentrated to tert-butyl 4-(4-aminophenyl)-2,2-dimethylpiperazine-1-carboxylate as light blackish solid (275 mg, 50.3%). ¹H NMR (400 MHz, DMSO-d₆): δ 6.617-6.596 (d, 2H), 6.501-6.480 (d, 2H), 4.508 (s, 2H), 3.525-3.499 (t, 2H), 2.999-2.973 (t, 2H) 1.411 (s, 9H), 1.351 (s, 6H).

### S. 1-(4-Aminophenyl)-N,N-dimethylpiperidin-3-amine

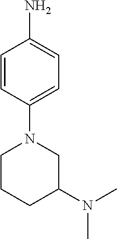 [SMILES: CN(C)C1CCCN(c2ccc(N)cc2)C1]

### Step 1: tert-Butyl 3-(dimethylamino)piperidine-1-carboxylate

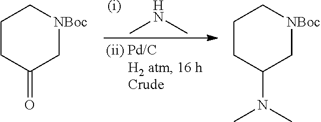

To a stirred solution of tert-butyl 3-oxopiperidine-1-carboxylate (3.0 g, 0.015 mol, 1.0 eq) in ethanol (50 mL) in a round bottomed flask at 0° C., N,N-dimethylamine (1.5 eq, 2M solution in THF) and acetic acid (1 mL) were added and stirred at rt for 2 h. To this mixture, 10% w/w Pd/C (50% wet, 750 mg) was added and stirred at rt overnight under hydrogen atmosphere. The mixture was filtered through a Celite® bed and washed with methanol (50 mL). The filtrate was concentrated to obtain tert-butyl 3-(dimethylamino)piperidine-1-carboxylate (2.1 g, crude) as pale yellow liquid. The product was used as such in the next step. Direct mass (M+H) m/z 229.2

### Step 2: N,N-dimethylpiperidin-3-amine hydrochloride

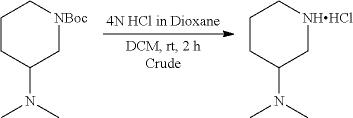

To a stirred solution of tert-butyl 3-oxopiperidine-1-carboxylate (2.0 g, 0.0087 mol, 1.0 eq) in DCM (30 mL) in a round bottomed flask at 0° C., 4N HCl in dioxane (6 mL) was added and stirred at rt for 2 h while monitoring by TLC. After completion of starting materials, the mixture was concentrated to obtain N,N-dimethylpiperidin-3-amine hydrochloride (2.4 g, crude) as pale yellow gum. This was used this material in next step without further purification.

### Step 3: N,N-dimethyl-1-(4-nitrophenyl)piperidin-3-amine

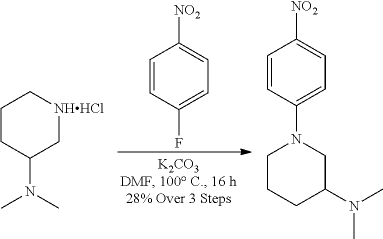

To a stirred solution of N,N-dimethylpiperidin-3-amine hydrochloride (2.3 g) in DMF (20 mL) in a round bottomed flask, K₂CO₃ (10.5 g, 8.0 eq), 1-fluoro-4-nitrobenzene (2.0 g, 0.0142 mol. 1.5 eq) were added and the mixture heated at 100° C. for 16 h. After TLC showed completion of starting material, the mixture was cooled and poured in ice cold water. The aqueous layer was extracted with ethyl acetate (3×100 mL), the organic layer was washed with water (3×50 mL), dried over sodium sulphate and concentrated to obtain crude product. The crude material was purified by Combiflash® flash chromatography with methanol in DCM as eluent. The product eluted at 4-5% methanol in DCM and the fractions with product were concentrated to obtain N,N-dimethyl-1-(4-nitrophenyl)piperidin-3-amine (1.05 g 28% over 3 steps) as pale yellow gum. Direct mass (M+H) m/z 250.2.

### Step 4: 1-(4-Aminophenyl)-N,N-dimethylpiperidin-3-amine

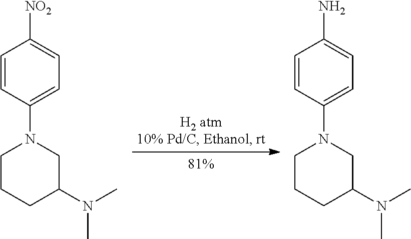 [SMILES: CN(C)C1CCCN(c2ccc([N+](=O)[O-])cc2)C1 ; CN(C)C1CCCN(c2ccc(N)cc2)C1]

To a stirred solution of N,N-dimethyl-1-(4-nitrophenyl)piperidin-3-amine (1.0 g, 0.004 mol, 1.0 eq) in ethanol (50 mL) in a round bottomed flask under nitrogen atmosphere, 10% w/w palladium on carbon (50% wet, 1.3 g) was added and the mixture was hydrogenated using a hydrogen balloon overnight. After TLC showed completion of starting material, the mixture was filtered through a Celite® bed and washed with methanol. The filtrate was concentrated to obtain 1-(4-aminophenyl)-N,N-dimethylpiperidin-3-amine (0.71 g, 81%) as brown oily liquid. Direct Mass (M+H) m/z 220.29.

### T. tert-butyl 4-(4-aminophenyl)-2-ethylpiperazine-1-carboxylate

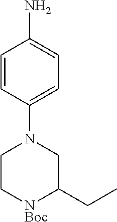 [SMILES: CCC1CN(c2ccc(N)cc2)CCN1C(=O)OC(C)(C)C]

### Step 1: 2-Ethylpiperazine

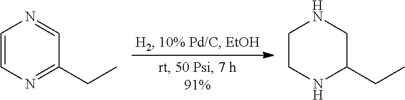

To a stirred mixture of 2-ethyl pyrazine (1.0 g, 0.0092 mol., 1.0 eq) in ethanol (20 ml), 10% Pd on carbon (0.1 g, 50% wet) was added and stirred at rt under H₂ atmosphere at 50 Psi for 7 h. After TLC showed completion of starting material, the mixture was filtered through Celite® bed and washed with ethanol (10 mL). The filtrate was concentrated to afford 2-ethyl piperazine (0.91 g, 91%).

### Step 2: tert-butyl 2-ethyl-4-(4-nitrophenyl) piperazine-1-carboxylate

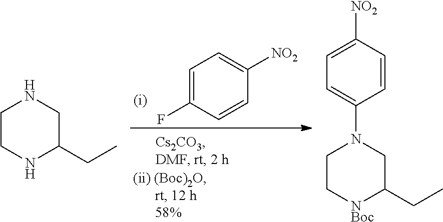

To a stirred solution of 2-ethyl piperazine (0.9 g, 1.0 eq) in DMF (40 mL), 4-fluoronitrobenzene (1.1 g, 1.2 eq) and Cs₂CO₃ were added and stirred at rt for 2 h. To this mixture, Boc-anhydride (1.9 mL, 1.0 eq) was added and continued stirring at rt for 12 h. The mixture was poured into ice water and extracted with EtOAc (2×60 mL). The organic layer was dried over anhydrous Na₂SO₄, filtered and concentrated to obtain crude compound. The resulting residue was purified by flash chromatography by using 230-400# silica gel and eluted with 10-11% EtOAc in hexane to obtain tert-butyl 2-ethyl-4-(4-nitrophenyl)piperazine-1-carboxylate (1.5 g, 59%) as pale yellow solid.

### Step 3: tert-butyl 4-(4-aminophenyl)-2-ethylpiperazine-1-carboxylate

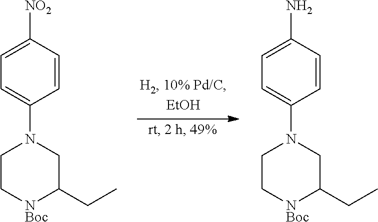 [SMILES: CCC1CN(c2ccc([N+](=O)[O-])cc2)CCN1C(=O)OC(C)(C)C ; CCC1CN(c2ccc(N)cc2)CCN1C(=O)OC(C)(C)C]

To a solution of tert-butyl 2-ethyl-4-(4-nitrophenyl)piperazine-1-carboxylate (1.5 g, 1.0 eq) in methanol (50 mL) at rt, 10% Pd/C (0.10 g, 50% wet) was added and stirred under hydrogen balloon at rt for 2 h. After TLC showed completion of starting material, the mixture was filtered through Celite® bed and washed with methanol (50 mL). The filtrate was concentrated to obtain crude residue. The resulting residue was purified by Combiflash™ chromatography (10 g snap with silica gel 230-400#) and product was eluted in 18-19% EtOAc in n-hexane. The fractions with pure product were concentrated to get tert-butyl 4-(4-aminophenyl)-2-ethylpiperazine-1-carboxylate (0.71 g, 49%) as pale brown gum. ¹H NMR (400 MHz, DMSO-d₆): δ 6.667 (d, 2H), 6.487 (d, 2H), 4.607 (s, 2H), 3.942 (m, 1H), 3.839 (d, 1H), 3.210 (d, 2H), 3.008 (brs, 1H), 2.364 (t, 1H), 1.667 (m, 2H), 1.406 (s, 9H), 0.822 (t, 3H).

### U. tert-butyl 4-(2-(4-aminophenoxy)ethyl)piperazine-1-carboxylate

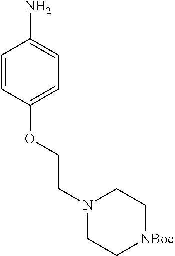 [SMILES: CC(C)(C)OC(=O)N1CCN(CCOc2ccc(N)cc2)CC1]

### Step 1: 1-(2-bromoethoxy)-4-nitrobenzene

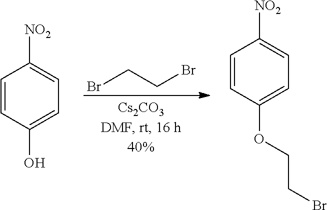

To a stirred solution of 4-nitrophenol (10 g, 0.071 mol) and Cs₂CO₃ (69 g, 3 eq) in DMF, 1,2-dibromoethane (40 mL, 0.215 mol, 3 eq) was added drop wise and the mixture stirred at rt for 16 h. After TLC showed completion of starting material, the mixture was poured into ice cold water and extracted with ethyl acetate (3×100 mL). The organic layer was washed with water (3×50 mL), dried over Na₂SO₄ and concentrated to obtain crude compound which was purified by flash column chromatography with 230-400# silica gel using ethyl acetate in hexane as solvent system. The product eluted at 5% EtOAc in hexane and the fractions with product were concentrated to afford 1-(2-bromoethoxy)-4-nitrobenzene (7 g, 40%) as white solid. ¹H NMR (400 MHz, CDCl₃): δ 8.22 (d, 2H), 6.98 (d, 2H), 4.38 (t, 2H), 3.67 (t, 2H). LCMS calculated for (M) 493.58 and found (M+H) 494.23 LCMS showed 90.03% purity.

### Step 2: tert-butyl 4-(2-(4-nitrophenoxy)ethyl)piperazine-1-carboxylate

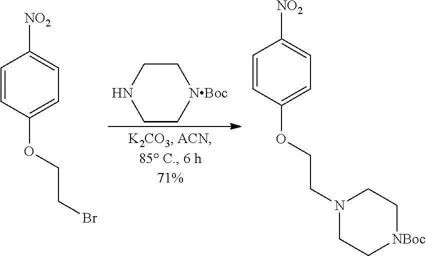

To a stirred solution of 1-(2-bromoethoxy)-4-nitrobenzene (2 g, 0.0081 mol) in DMF in a round bottomed flask, K₂CO₃ (3.3 g, 0.024 mol) and tert-butyl piperazine-1-carboxylate (1.82 g, 0.0097 mol. 1.2 eq) were added and the temperature raised to 85° C. for 6 h, while monitoring by TLC. After completion of starting material, the mixture was cooled and poured in ice cold water. Aqueous layer was extracted with ethyl acetate (3×100 mL). The organic layer was washed with water (3×50 mL), dried over Na₂SO₄ and concentrated to obtain crude compound which was purified by flash column chromatography with 230-400 # silica gel using ethyl acetate in hexane as solvent system. The product was eluted at 5% EtOAc in hexane and the fraction with product upon concentration afforded tert-butyl 4-(2-(4-nitrophenoxy)ethyl)piperazine-1-carboxylate (2 g, 71%) as white solid. ¹H NMR (400 MHz, CDCl₃): δ 8.20 (d, 2H), 6.96 (d, 2H), 4.19 (t, 2H), 3.45 (t, 4H). 2.85 (t, 2H), 2.52 (t, 4H), 1.46 (s, 9H). LCMS calculated for (M) 351.40 and found (M+H) 352.49 LCMS showed 90.03% purity.

### Step 3: tert-butyl 4-(2-(4-aminophenoxy)ethyl)piperazine-1-carboxylate

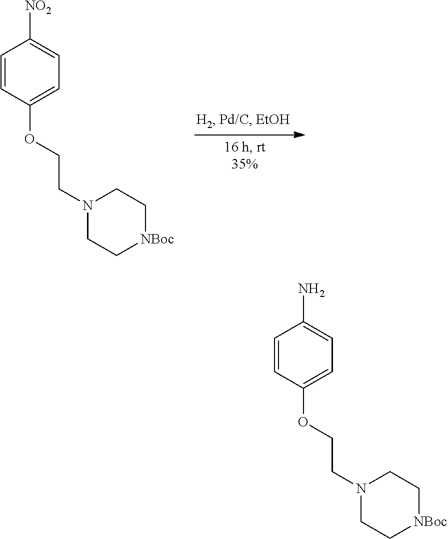 [SMILES: CC(C)(C)OC(=O)N1CCN(CCOc2ccc([N+](=O)[O-])cc2)CC1 ; CC(C)(C)OC(=O)N1CCN(CCOc2ccc(N)cc2)CC1]

To the solution of tert-butyl 4-(2-(4-nitrophenoxy)ethyl)piperazine-1-carboxylate (2 g, 1.0 eq,) in ethanol (80 mL), 10% Pd/C (1.0 g, 50% wet) was added and stirred under hydrogen balloon at rt for 16 h. After completion of starting material, the mixture was filtered through a Celite® bed and washed with methanol (20 mL). The filtrate was concentrated to obtain tert-butyl 4-(2-(4-aminophenoxy)ethyl)piperazine-1-carboxylate (1 g, 35%) as off-white solid. ¹H NMR (400 MHz, DMSO-d₆): δ 6.63 (d, 2H), 6.48 (d, 2H), 4.59 (s, 2H), 3.91 (t, 2H), 3.30 (t, 4H), 2.64 (d, 2H), 2.40 (t, 4H), 1.38 (s, 9H). LCMS calculated for (M) 321.42 and found (M+H) 322.05 LCMS showed 89.04% purity.

### V. 3-(2-(dimethylamino)ethoxy)aniline

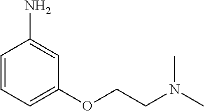

### Step 1: N,N-dimethyl-2-(3-nitrophenoxy)ethan-1-amine

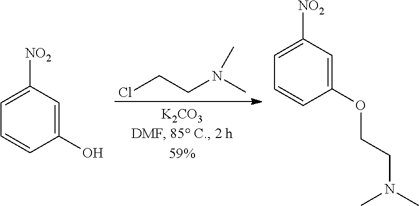

To a stirred solution of 3-nitrophenol (0.5 g) in DMF (10 mL), K₂CO₃ (1.48 g, 0.010 mol, 3.0 eq), 2-chloro-N,N-dimethylethan-1-amine (0.77 g, 1.5 eq) were added and the mixture was stirred at 85° C. for 2 h. After completion of starting material on TLC, the mixture was poured in ice cold water and extracted with ethyl acetate (3×250 mL). The organic layer was washed with water (3×100 mL), dried over Na₂SO₄ and concentrated to obtain crude material which was purified by flash chromatography using 65% ethyl acetate in hexane as eluent to obtain N,N-dimethyl-2-(3-nitrophenoxy)ethan-1-amine (0.45 g, 59%) as brown liquid. ¹H NMR (400 MHz, DMSO-d₆): δ 7.82 (d, 1H), 7.54 (s, 1H), 7.42 (t, 1H), 7.26-7.24 (m, 1H), 4.14 (t, 2H), 2.77 (t, 2H), 2.3 (s, 6H).

### Step 2: 3-(2-(dimethylamino)ethoxy)aniline

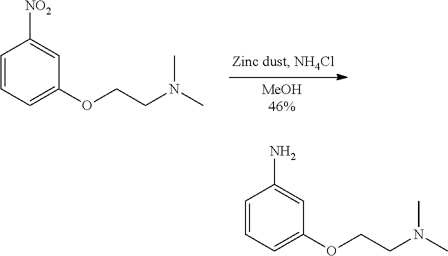

To a stirred solution of N,N-dimethyl-2-(3-nitrophenoxy)ethan-1-amine (0.45 g) in methanol (15 mL) at 0°-10° C., zinc dust (0.7 g, 5 eq) and ammonium chloride (0.7 g, 5 eq) were added portion wise and the reaction was stirred at rt for 5 h. After completion of starting material, the mixture was filtered through Celite® bed, and washed with methanol (20 mL). The organic layer was concentrated under reduced pressure to get crude material which was purified by Combiflash™ chromatography (230-400# silica gel) using 5% methanol in DCM as eluent to obtain 3-(2-(dimethylamino)ethoxy)aniline (0.18 g, 46%) as brown semisolid. ¹H NMR (400 MHz, DMSO-d₆): δ 7.06 (t, 1H), 6.34 (d, 1H), 6.27 (d, 2H), 7.26-7.24 (m, 1H), 4.03 (t, 2H), 3.6 (s, 2H), 2.71 (t, 2H), 2.3 (s, 6H).

### X. tert-butyl 4-(6-aminopyridin-3-yl)-3-oxopiperazine-1-carboxylate

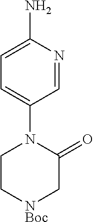 [SMILES: CC(C)(C)OC(=O)N1CCN(c2ccc(N)nc2)C(=O)C1]

### Step 1: tert-butyl 4-(6-aminopyridin-3-yl)-3-oxopiperazine-1-carboxylate

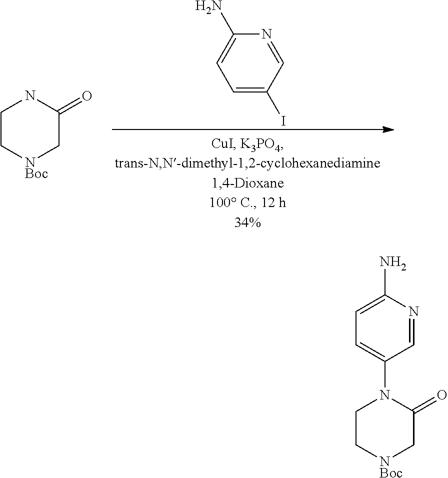 [SMILES: CC(C)(C)OC(=O)N1CCNC(=O)C1 ; Nc1ccc(I)cn1 ; CC(C)(C)OC(=O)N1CCN(c2ccc(N)nc2)C(=O)C1]

To a solution of tert-butyl 3-oxopiperazine-1-carboxylate (0.5 g, 1.0 eq) and 5-iodopyridin-2-amine (0.539 g, 1.2 eq) in 1,4-dioxane (25 mL), CuI (0.129 g, 0.3 eq), trans-N,N′-dimethyl-1,2-cyclohexanediamine (0.077 g, 0.2 eq) and K₃PO₄ (1.4 g, 3.0 eq) were added and the contents refluxed at 100° C. for 16 h. After completion of starting material, the mixture was quenched with water (40 mL) and extracted with EtOAc (2×100 ml). The organic layer was dried over anhydrous Na₂SO₄, filtered and concentrated. The resulting residue was purified on column chromatography on silica gel (100-200#) using 2% methanol in DCM as eluent to obtain tert-butyl 4-(6-aminopyridin-3-yl)-3-oxopiperazine-1-carboxylate (0.252 g, 34%). LCMS calculated for (M) 293.12 and found (M+H) 293.21. LCMS showed 98.56% purity.

### Y. 5-morpholinopyridin-2-amine

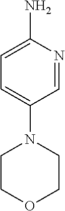 [SMILES: Nc1ccc(N2CCOCC2)cn1]

### Step 1: 4-(6-nitropyridin-3-yl)morpholine

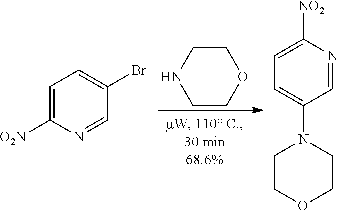

A mixture of 5-bromo-2-nitropyridine (1.0 g, 1.0 eq) and morpholine (8 mL) was irradiated with microwave radiation (Biotage) at 110° C. for 30 min. After completion of starting material on TLC, the mixture was concentrated to obtain crude material which was purified by Combiflash™ chromatography (silica gel, 230-400#) using 3% methanol in DCM as eluent to obtain 4-(6-nitropyridin-3-yl)morpholine (0.7 g, 68.6%) as yellow solid. LCMS calculated for (M) 209.08 and found (M+H) 210.08, LCMS showed 99.64% purity.

### Step 2: 5-morpholinopyridin-2-amine

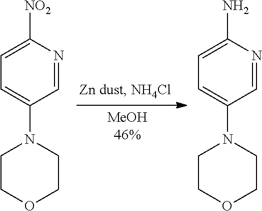 [SMILES: O=[N+]([O-])c1ccc(N2CCOCC2)cn1 ; Nc1ccc(N2CCOCC2)cn1]

To a stirred solution of 4-(6-nitropyridin-3-yl)morpholine (1.0 g, 1.0 eq) in methanol (20 mL) at 0°-10° C., zinc dust (1.5 g, 5.0 eq) and ammonium chloride (1.29 g, 5.0 eq) were added and the reaction was stirred at rt for 5 h. After completion, the mixture was filtered through a Celite® bed, washed with methanol. The organic layer was concentrated under reduced pressure to provide crude material which was purified by Combiflash® chromatography using 5% methanol in DCM as eluent to obtain 5-morpholinopyridin-2-amine (0.18 g, 46%) as violent solid. ¹H NMR (400 MHz, DMSO-d₆): δ 8.02 (s, 1H), 7.59 (d, 1H), 6.43 (s, 1H), 5.75 (s, 1H), 5.50 (s, 2H), 3.70 (s, 4H), 2.90 (s, 4H).

### Z. N-(4-aminophenyl)isobutyramide

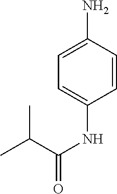

### Step 1: N-(4-nitrophenyl)isobutyramide

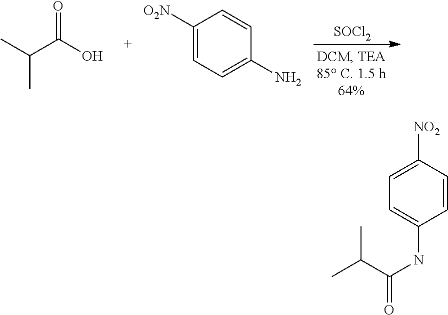

To a stirred solution of an isobutyric acid (1.2 g, 0.0136 mol) in dry diethylether (10 mL) at 0° C., thionyl chloride (8 mL) was added and the mixture was heated at 85° C. for 1.2 h. After completion of the reaction, excess of thionyl chloride was removed under reduced pressure and the crude product obtained was dissolved in DCM (10 mL) and a mixture of p-nitroaniline (2.06 g, 0.014 mol) and triethylamine (4.5 mL) in DCM (10 mL) was added and stirred for 30 min at rt. After completion of starting material on TLC, the mixture was quenched with ice and poured into ice cold water. The aqueous layer was extracted with ethyl acetate (3×250 mL). The organic layer was washed with water (3×100 mL), dried over Na₂SO₄ and concentrated to obtain crude material which was triturated with pentane to obtain N-(4-nitrophenyl)isobutyramide (1.8 g, 64.2%) as brown solid. ¹H NMR (400 MHz, DMSO-d₆): δ 10.46 (s, 1H), 8.21 (s, 2H), 7.84 (s, 2H), 1.12 (s, 6H).

### Step 2: N-(4-aminophenyl)isobutyramide

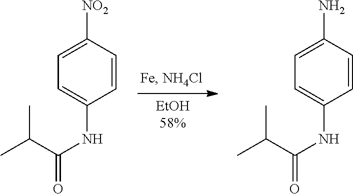

To a stirred solution of N-(4-nitrophenyl)isobutyramide (1.8 g, 0.0086 mol) in ethanol (25 mL), iron powder (2.4 g, 0.0216 mol) and ammonium chloride (0.23 g, 0.0043 mol) were added slowly and the reaction was stirred at 85° C. for 2 h. After completion of the reaction, the reaction was filtered through a Celite® bed and washed with methanol. The organic layer was concentrated under reduced pressure to provide crude material which was purified by washing with diethyl ether to obtain N-(4-aminophenyl)isobutyramide (0.70 g, 58%) as brown solid. ¹H NMR (400 MHz, DMSO-d₆): δ 9.37 (s, 1H), 7.22 (s, 2H), 6.4 (s, 2H), 4.8 (s, 2H), 1.06 (s, 6H).

### AA. 3-morpholinoaniline

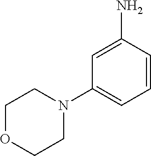 [SMILES: Nc1cccc(N2CCOCC2)c1]

### Step 1: 4-(3-nitrophenyl)morpholine

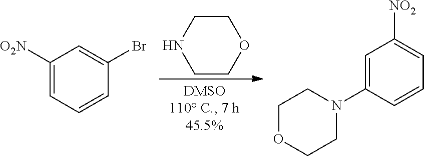

A solution of 1-fluoro-3-nitrobenzene (1.5 g, 0.0106 mol) and morpholine (4.6 g, 0.0530 mol) in DMSO (15 mL) was heated at 110° C. for 7 h. After completion of starting materials, as evidenced by TLC, the mixture was quenched with ice and poured into ice cold water. The aqueous layer was extracted with ethyl acetate (3×250 mL). The organic layer was washed with water (3×100 mL), dried over Na₂SO₄ and concentrated to obtain crude material which was filtered after trituration with pentane (10 mL) to obtain 4-(3-nitrophenyl)morpholine (1.0 g, 45.5%) as yellow solid. LCMS calculated for (M) 208.04 and found (M+H) 209.06, LCMS showed 99.42% purity.

### Step 2: 3-Morpholinoaniline

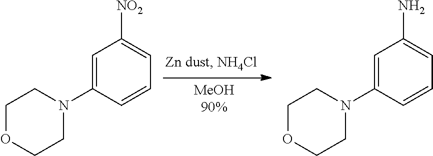 [SMILES: O=[N+]([O-])c1cccc(N2CCOCC2)c1 ; Nc1cccc(N2CCOCC2)c1]

To a stirred solution of 4-(6-nitropyridin-3-yl)morpholine (0.9 g, 0.0043 mol) in methanol (10 mL) was cooled to 0°-10° C. and zinc dust (1.4 g, 0.0216 mol) and ammonium chloride (1.16 g, 0.0216 mol) were added. The reaction was stirred at rt for 3 h. After completion, the mixture was filtered through a Celite® bed and washed with methanol. The organic layer was concentrated under reduced pressure to provide crude material which was purified by Combiflash™ chromatography using 5% methanol/DCM as eluent to obtain 3-morpholinoaniline (0.70 g, 90%) as violet color solid. ¹H NMR (400 MHz, DMSO-d₆): δ 6.86 (t, 2H), 6.12 (s, 2H), 6.056 (s, 1H), 5.6 (d, 1H), 6.43 (s, 1H), 3.70 (s, 4H), 2.90 (s, 4H).

### BB. (S)-tert-butyl 4-(6-aminopyridin-3-yl)-2-methylpiperazine-1-carboxylate

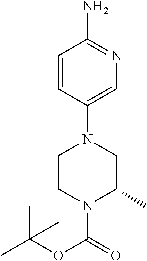 [SMILES: C[C@H]1CN(c2ccc(N)nc2)CCN1C(=O)OC(C)(C)C]

### Step 1: (S)-tert-butyl 2-methyl-4-(6-nitropyridin-3-yl)piperazine-1-carboxylate

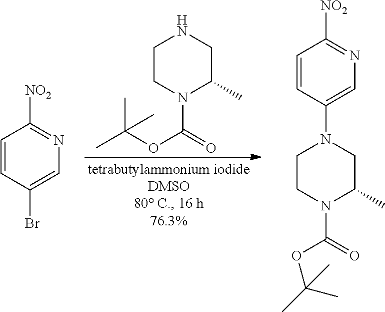

To a solution of 5-bromo-2-nitropyridine (2 g, 9.85 mmol) in DMSO (10 mL) was added tetrabutylammonium iodide (3.63 g, 9.85 mmol), followed by (S)-tert-butyl 2-methylpiperazine-1-carboxylate (3.94 g, 19.70 mmol), and the mixture was heated to 80° C. overnight. The mixture was diluted with ice cold water (50 mL), extracted in EtOAc (100 mL), dried over sodium sulfate, filtered and evaporated under reduced pressure to obtain crude product. The crude product was purified by 100-200 silica gel (using 27% EtOAc/hexane) to give (S)-tert-butyl 2-methyl-4-(6-nitropyridin-3-yl)piperazine-1-carboxylate (2.42 g, 76.3% yield); ¹H NMR (400 MHz, DMSO-d₆): δ 8.185 (d, J=4 Hz, 1H), 8.13 (d, J=8 Hz, 1H), 7.42-7.39 (m, 1H), 3.87 (s, 3H), 3.3 (s, 1H), 3.27 (s, 2H), 1.4 (s, 9H), 1.3 (s, 2H), 1.095 (d, J=2 Hz, 3H), LCMS m/z calcd for [M+H]⁺ 322.36. found 323.2.

### Step 2: (S)-tert-butyl 4-(6-aminopyridin-3-yl)-2-methylpiperazine-1-carboxylate

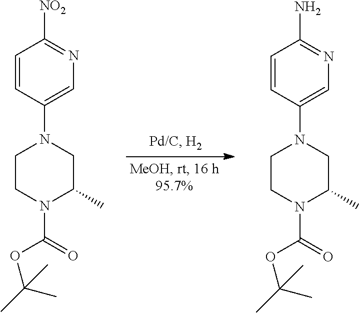 [SMILES: C[C@H]1CN(c2ccc([N+](=O)[O-])nc2)CCN1C(=O)OC(C)(C)C ; C[C@H]1CN(c2ccc(N)nc2)CCN1C(=O)OC(C)(C)C]

To a solution of (S)-tert-butyl 2-methyl-4-(6-nitropyridin-3-yl)piperazine-1-carboxylate (2.42 g, 7.51 mmol) in MeOH (50 mL) purged with argon was added Pd/C (0.25 g, 10%). The mixture was charged with a H₂ bladder and the mixture was stirred at rt overnight. The mixture was filtered through a Celite® bed using MeOH (100 mL) and the solvent evaporated under reduced pressure to give (S)-tert-butyl 4-(6-aminopyridin-3-yl)-2-methylpiperazine-1-carboxylate (2.01 g, 95.7% yield); ¹H NMR (400 MHz, DMSO-d₆): δ 7.745 (d, 1H), 713 (q, J=4 Hz, 1H), 6.47 (d, J=12 Hz, 1H), 3.93 (d, J=8 Hz, 1H), 3.235 (d, J=8 Hz, 2H), 2.84 (d, J=12 Hz, 2H), 2.66 (t, J=12 Hz, 2H), 2.32 (s, 9H), 1.34 (d, J=12 Hz, 3H), LCMS m/z calcd for [M+H]⁺ 292.38.42. found 293.2.

### Example 231

### N-(4-(piperazin-1-yl)phenyl)-1-(quinolin-8-ylsulfonyl)-1H-pyrazolo[3,4-d]pyrimidin-6-amine

### Step 1: tert-Butyl 4-(4-((1-(quinolin-8-ylsulfonyl)-1H-pyrazolo[3,4-d]pyrimidin-6-yl)amino)phenyl)piperazine-1-carboxylate

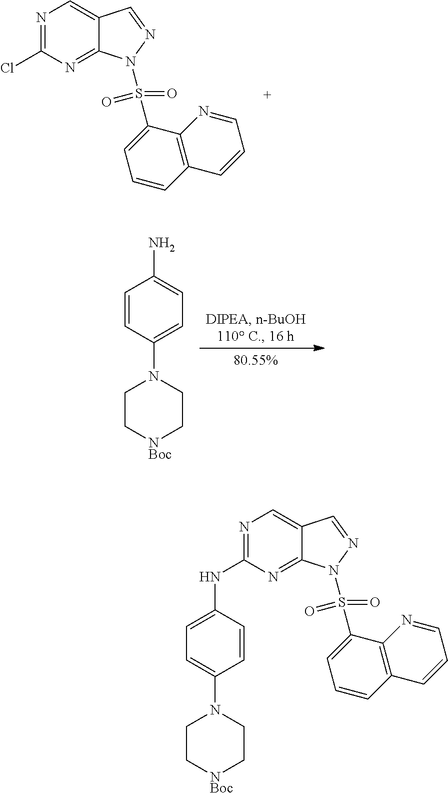 [SMILES: O=S(=O)(c1cccc2cccnc12)n1ncc2cnc(Cl)nc21 ; CC(C)(C)OC(=O)N1CCN(c2ccc(N)cc2)CC1 ; CC(C)(C)OC(=O)N1CCN(c2ccc(Nc3ncc4cnn(S(=O)(=O)c5cccc6cccnc56)c4n3)cc2)CC1]

A stirred mixture of 8-((6-chloro-1H-pyrazolo[3,4-d]pyrimidin-1-yl)sulfonyl)quinoline (0.3 g, 0.00086 mol, 1.0 eq), tert-butyl 4-(4-aminophenyl)piperazine-1-carboxylate (0.95 eq), DIPEA (3.0 eq) in n-BuOH (10 mL) in a sealed vial was heated at 110° C. for 16 h. After TLC showed completion of the starting material, the mixture was cooled to rt, poured into water and extracted with ethyl acetate (2×100 mL). The organic layer was dried over Na₂SO₄ and the solvent distilled off to get the crude product. The crude product was purified through Combiflash® chromatography (silica gel) using MeOH in DCM as eluent. The desired compound was eluted at 0.8% MeOH in DCM and the concentration of the pure fractions provided tert-butyl 4-(4-((1-(quinolin-8-ylsulfonyl)-1H-pyrazolo[3,4-d]pyrimidin-6-yl)amino)phenyl)piperazine-1-carboxylate as white solid (410 mg, 80.55%). ¹H NMR (400 MHz, DMSO-d₆): δ 10.028 (s, 1H), 8.938 (s, 1H), 8.649-8.635 (m, 2H), 8.502-8.481 (d, 1H), 8.422-8.401 (d, 1H), 8.265 (s, 1H), 7.907-7.868 (m, 1H), 7.670-7.648 (d, 2H), 7.694-7.689 (d, 1H), 7.589-7.558 (m, 2H), 6.940-6.918 (d, 2H), 3.471 (s, 4H), 3.068-3.057 (d, 4H), 1.424 (s, 9H). LCMS calculated for (M) 584.20 and found (M−H) 585.21.

### Step 2: N-(4-(piperazin-1-yl)phenyl)-1-(quinolin-8-ylsulfonyl)-1H-pyrazolo[3,4-d]pyrimidin-6-amine

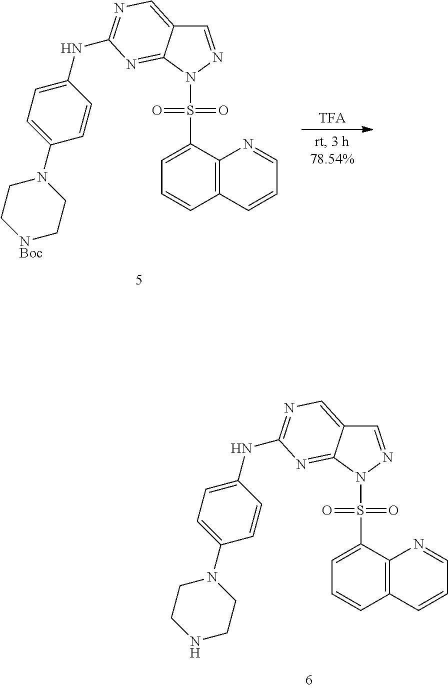 [SMILES: CC(C)(C)OC(=O)N1CCN(c2ccc(Nc3ncc4cnn(S(=O)(=O)c5cccc6cccnc56)c4n3)cc2)CC1 ; O=S(=O)(c1cccc2cccnc12)n1ncc2cnc(Nc3ccc(N4CCNCC4)cc3)nc21]

In a round bottomed flask, tert-butyl 4-(4-((1-(quinolin-8-ylsulfonyl)-1H-pyrazolo[3,4-d]pyrimidin-6-yl)amino)phenyl)piperazine-1-carboxylate (0.4 g) was taken up in DCM under N₂ atmosphere. The mixture was cooled to 0° C. and then TFA (3.5 mL) was added. The mixture was warmed to rt and stirred for 3 h. The reaction was then checked using TLC. The solvent was removed under reduced vacuum, the residue washed with ether and DCM added. The solution was basified using freshly prepared NaHCO₃ solution, and then extracted with DCM. The organic layer was dried over Na₂SO₄ and the solvent distilled to provide the desired N-(4-(piperazin-1-yl)phenyl)-1-(quinolin-8-ylsulfonyl)-1H-pyrazolo[3,4-d]pyrimidin-6-amine (260 mg, 78.54%). ¹H NMR (400 MHz, DMSO-d₆): δ 10.017 (s, 1H), 8.928 (s, 1H), 8.703-8.640 (m, 2H), 8.499-8.478 (d, 1H), 8.417-8.397 (d, 1H), 8.255 (s, 1H), 7.889-7.849 (m, 1H), 7.648-7.626 (d, 2H), 7.587-7.556 (m, 1H), 6.924-6.875 (m, 2H), 3.031 (s, 4H), 2.882-2.871 (d, 4H), LCMS calculated for (M+H) 487.20 found 487.17.

### Example 330

### N-(2-Methoxy-4-(piperazin-1-yl)phenyl)-1-(quinolin-8-ylsulfonyl)-1H-pyrrolo[3,2-c]pyridin-6-amine

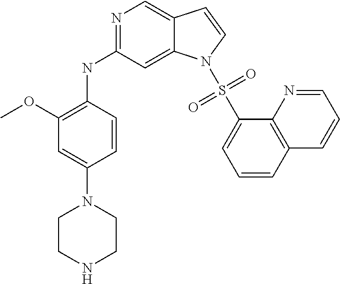 [SMILES: COc1cc(N2CCNCC2)ccc1Nc1cc2c(ccn2S(=O)(=O)c2cccc3cccnc23)cn1]

### Step 1: tert-Butyl 4-(3-methoxy-4-((1-(quinolin-8-ylsulfonyl)-1H-pyrrolo[3,2-c]pyridin-6-yl)amino)phenyl)piperazine-1-carboxylate

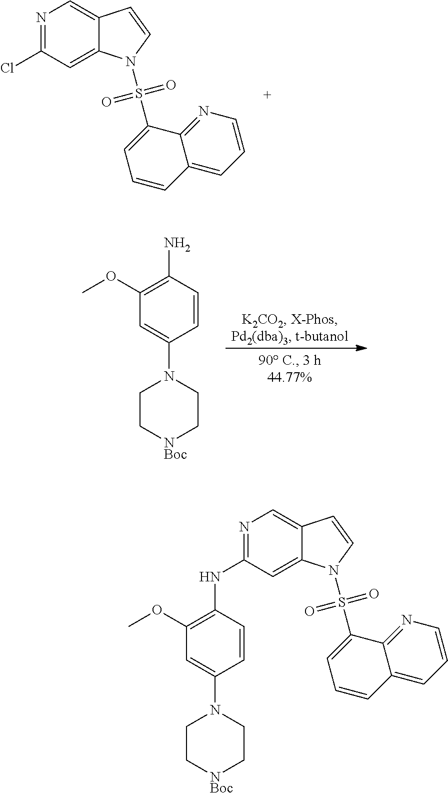 [SMILES: O=S(=O)(c1cccc2cccnc12)n1ccc2cnc(Cl)cc21 ; COc1cc(N2CCN(C(=O)OC(C)(C)C)CC2)ccc1N ; COc1cc(N2CCN(C(=O)OC(C)(C)C)CC2)ccc1Nc1cc2c(ccn2S(=O)(=O)c2cccc3cccnc23)cn1]

A stirred mixture of 8-((6-chloro-1H-pyrrolo[3,2-c]pyridin-1-yl)sulfonyl)quinoline (3.0 g, 0.00874 mol, 1.0 eq), tert-butyl 4-(4-amino-3-methoxyphenyl)piperazine-1-carboxylate (2.1 g, 0.00699, 0.8 eq), K₂CO₃ (3.60 g, 3.0 eq), and t-butanol (100 mL) in a round bottomed flask was degassed with an argon balloon for 20 min at rt, and then X-phos (0.1 eq), Pd₂(dba)₃ (0.05 eq) were added and again degassed for 10 min. The mixture was heated under a reflux condenser at 90° C. for 6 h while monitoring by TLC. After completion of starting material, the mixture was cooled to rt, diluted with water (150 mL) and extracted with ethyl acetate (3×150 mL). The separated organic layer was washed with brine solution (50 mL), dried over anhydrous sodium sulphate and concentrated under reduced pressure to obtain crude product. The crude product was purified by column chromatography on basic alumina using EtOAc in hexane as eluent. The product eluted at 70% EtOAc in hexane and the concentration of pure fractions provided tert-butyl 4-(3-methoxy-4-((1-(quinolin-8-ylsulfonyl)-1H-pyrrolo[3,2-c]pyridin-6-yl)amino)phenyl)piperazine-1-carboxylate as yellow solid tert-butyl 4-(3-methoxy-4-((1-(quinolin-8-ylsulfonyl)-1H-pyrrolo[3,2-c]pyridin-6-yl)amino)phenyl)piperazine-1-carboxylate (1.50 g, 44.77%). ¹H NMR (400 MHz, CDCl₃): δ 8.954 (s, 1H), 8.459 (s, 1H), 8.441 (d, 1H), 8.366 (d, 1H), 8.188 (s, 1H), 8.168 (t, 1H), 7.833 (d, 1H), 7.64 (s, 1H), 7.488 (t, 1H), 7.467 (d, 1H), 6.666 (s, 1H), 6.564 (t, 2H), 6.506 (d, 1H), 3.842 (s, 3H), 3.628 (t, 4H), 3.108 (t, 4H), 1.574 (s, 9H).

### Step 2: N-(2-methoxy-4-(piperazin-1-yl)phenyl)-1-(quinolin-8-ylsulfonyl)-1H-pyrrolo[3,2-c]pyridin-6-amine

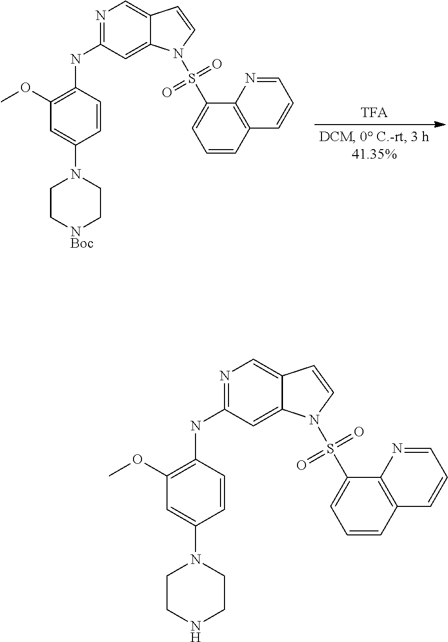 [SMILES: COc1cc(N2CCN(C(=O)OC(C)(C)C)CC2)ccc1Nc1cc2c(ccn2S(=O)(=O)c2cccc3cccnc23)cn1 ; COc1cc(N2CCNCC2)ccc1Nc1cc2c(ccn2S(=O)(=O)c2cccc3cccnc23)cn1]

To a stirred solution of tert-butyl 4-(3-methoxy-4-((1-(quinolin-8-ylsulfonyl)-1H-pyrrolo[3,2-c]pyridin-6-yl)amino)phenyl)piperazine-1-carboxylate (1.6 g, 0.0026 mol, 1.0 eq) in DCM (75 mL) at 0° C., TFA (3 mL) was added drop wise and the mixture stirred at rt for 3 h while monitoring by TLC. After completion of the starting material, the solvent was distilled off under vacuum, the resulting residue was washed with ether (2×15 mL), the solution neutralised with 10% sodium bicarbonate solution and extracted with DCM (3×50 mL). The organic layer was washed with brine, dried over anhydrous sodium sulphate and concentrated to obtain crude product. The crude product was purified by column chromatography on basic alumina using methanol in DCM as the eluent. The product eluted at 5% methanol in DCM and concentration of pure fractions afforded N-(2-methoxy-4-(piperazin-1-yl)phenyl)-1-(quinolin-8-ylsulfonyl)-1H-pyrrolo[3,2-c]pyridin-6-amine as yellow solid (0.55 g, 41.35%). ¹H NMR (400 MHz, DMSO-d₆): δ 8.962 (brs, 1H), 8.52 (s, 1H), 8.424 (d, 1H), 8.406 (d, 1H), 8.271 (s, 1H), 8.767 (t, 1H), 7.769 (d, 1H), 7.72 (s, 1H), 7.68 (t, 1H), 7.431 (d, 1H), 6.986 (s, 1H), 6.632 (t, 2H), 6.448 (d, 1H), 3.713 (s, 3H), 3.052 (t, 4H), 2.885 (t, 4H). LCMS calculated for (M) 514.18 and found (M+H) 515.33. LCMS showed 98.97% purity.

### Example 338

### N-(2-methoxy-4-(piperazin-1-yl)phenyl)-1-(quinolin-8-ylsulfonyl)-1H-pyrazolo[4,3-c]pyridin-6-amine

### Step 1: tert-Butyl 4-(3-methoxy-4-((1-(quinolin-8-ylsulfonyl)-1H-pyrazolo[4,3-c]pyridin-6-yl)amino)phenyl)piperazine-1-carboxylate

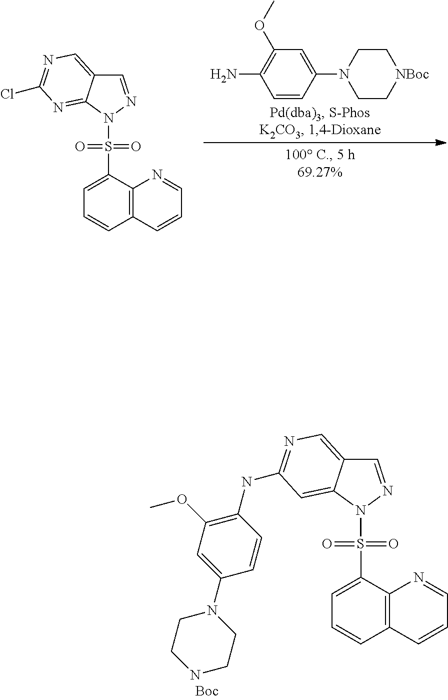 [SMILES: O=S(=O)(c1cccc2cccnc12)n1ncc2cnc(Cl)nc21 ; COc1cc(N2CCN(C(=O)OC(C)(C)C)CC2)ccc1N ; COc1cc(N2CCNCC2)ccc1Nc1cc2c(cn1)cnn2S(=O)(=O)c1cccc2cccnc12]

A stirred mixture of 8-((6-chloro-1H-pyrazolo[4,3-c]pyridin-1-yl)sulfonyl)quinoline (3.5 g, 0.01017 mol, 1.0 eq), tert-butyl 4-(4-amino-3-methoxyphenyl)piperazine-1-carboxylate (3.1 g, 1.0 eq), K₂CO₃ (4.2 g, 3 eq) in 1,4-dioxane (150 mL) in a round bottomed flask was degassed using an argon balloon for 30 min and X-Phos (0.484 g, 0.1 eq) and Pd₂(dba)₃ (0.961 g, 0.1 eq) were added to this mixture. The contents were heated at 85° C. for 16 h while monitoring by TLC. After completion of starting material, the mixture was cooled to rt and filtered through a Celite® bed. The filtrate was diluted with water and the aqueous layer was extracted with ethyl acetate. The organic layer was concentrated and the resulting residue was purified by flash Combiflash® column chromatography using 100-200# silica gel with 85% EtOAc in hexane as the eluting solvent to obtain tert-butyl 4-(3-methoxy-4-((1-(quinolin-8-ylsulfonyl)-1H-pyrazolo[4,3-c]pyridin-6-yl)amino)phenyl)piperazine-1-carboxylate as pale brown solid (0.230 g, 69.27%). ¹H NMR (400 MHz, DMSO-d₆): δ 8.816 (s, 1H), 8.616 (s, 1H), 8.535 (d, 1H), 8.373 (m, 3H), 8.205 (s, 1H), 7.822 (m, 1H), 7.593 (m, 1H), 7.501 (m, 1H), 7.0212 (s, 1H), 6.692 (d, 1H), 6.545 (d, 1H), 3.786 (s, 2H), 3.404 (brs, 4H), 3.110 (brs, 4H), 1.418 (s, 9H).

### Step 2: N-(2-methoxy-4-(piperazin-1-yl)phenyl)-1-(quinolin-8-ylsulfonyl)-1H-pyrazolo[4,3-c]pyridin-6-amine

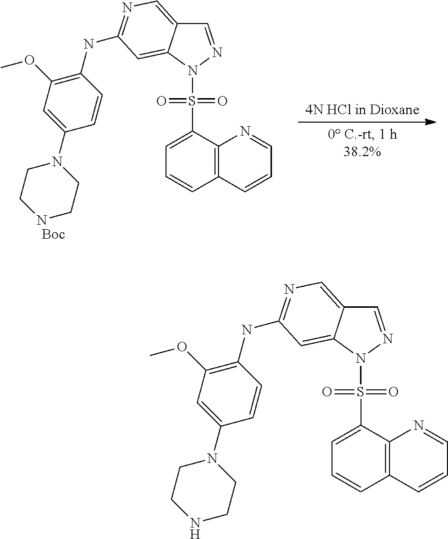 [SMILES: COc1cc(N2CCNCC2)ccc1Nc1cc2c(cn1)cnn2S(=O)(=O)c1cccc2cccnc12 ; COc1cc(N2CCNCC2)ccc1Nc1cc2c(cn1)cnn2S(=O)(=O)c1cccc2cccnc12]

To a stirred solution of tert-butyl 4-(3-methoxy-4-((1-(quinolin-8-ylsulfonyl)-1H-pyrazolo[4,3-c]pyridin-6-yl)amino)phenyl)piperazine-1-carboxylate (0.23 g, 1.0 eq) in 1,4-dioxane in a round bottomed flask at 0° C., 4N HCl in dioxane was added drop wise at 0° C., and the mixture stirred at rt for 2 h while monitoring by TLC. After completion of starting material, the mixture was poured in water and extracted with ethyl acetate. The aqueous layer was basified with saturated bicarbonate solution and extracted with DCM. The organic layer was washed with water, brine solution, dried over Na₂SO₄ and concentrated. The resulting residue was purified by Combiflash® flash column chromatography (basic alumina) using MeOH in DCM as eluting solvent. The product eluted at 2% MeOH in DCM and concentration of the pure fractions provided N-(2-methoxy-4-(piperazin-1-yl)phenyl)-1-(quinolin-8-ylsulfonyl)-1H-pyrazolo[4,3-c]pyridin-6-amine as yellow solid (0.075 g, 38.25%). ¹H NMR (400 MHz, DMSO-d₆): δ 8.608 (s, 1H), 8.570 (m, 1H), 8.492 (d, 1H), 8.380 (t, 2H), 8.295 (s, 1H), 8.198 (s, 1H), 7.821 (t, 1H), 7.582 (m, 1H), 7.393 (d, 1H), 7.165 (s, 1H), 6.634 (s, 1H), 6.501 (d, 1H), 3.772 (s, 3H), 3.052 (brs, 4H), 2.830 (brs, 4H). LCMS calculated for (M+H) 516.6 and found 516.2.

### Example 448

### (R)—N-methyl-N-(2-((2-((4-(3-methylpiperazin-1-yl)phenyl)amino)-7H-pyrrolo[2,3-d]pyrimidin-7-yl)methyl)phenyl)methanesulfonamide

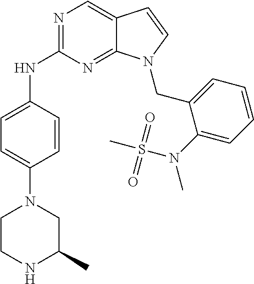 [SMILES: C[C@@H]1CN(c2ccc(Nc3ncc4ccn(Cc5ccccc5N(C)S(C)(=O)=O)c4n3)cc2)CCN1]

### Step 1: (R)-tert-butyl 2-methyl-4-(4-((7-(2-(N-methylmethylsulfonamido)benzyl)-7H-pyrrolo[2,3-d]pyrimidin-2-yl)amino)phenyl)piperazine-1-carboxylate

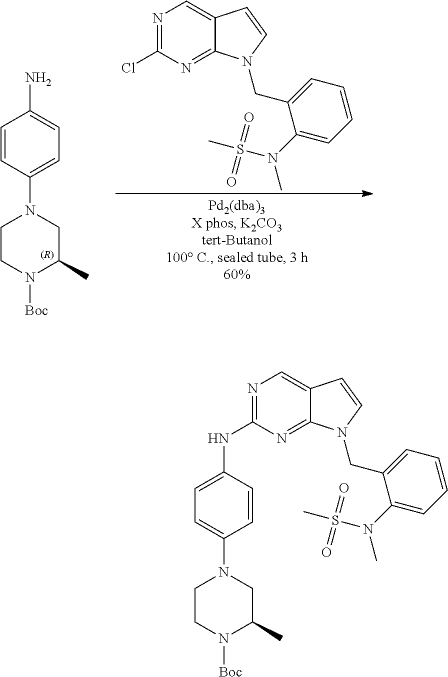

A stirred mixture of N-(2-((2-chloro-7H-pyrrolo[2,3-d]pyrimidin-7-yl)methyl)phenyl)-N-methylmethanesulfonamide (0.15 g, 0.42 mmol, 1.0 eq), (R)-tert-butyl 4-(4-aminophenyl)-2-methylpiperazine-1-carboxylate (0.15 g, 0.42 mmol, 0.9 eq) and K₂CO₃ (0.17 g, 1.28 mmol, 3.0 eq) in t-butanol (5.0 mL) in a vial was degassed using an argon balloon for 10 min. X-Phos (0.020 g, 0.042 mmol, 0.1 eq) and Pd₂(dba)₃ (0.019 g, 0.021 mmol, 0.05 eq) were added to this mixture and the solution continued degassing for another 10 min. The vial was closed and the contents heated at 90° C. for 3 h. After completion of the starting material, the mixture was quenched with water (30 mL) and extracted with EtOAc (2×50.0 mL). The organic layer was dried over anhydrous Na₂SO₄, filtered and concentrated. The resulting residue was purified by flash chromatography (Biotage® Isolera™ purifier, 25 g column) using EtOAc in hexane as eluent. The desired product eluted at 60% EtOAc in hexane. The fractions with product were concentrated to obtain (R)-tert-butyl 2-methyl-4-(4-((7-(2-(N-methylmethylsulfonamido)benzyl)-7H-pyrrolo[2,3-d]pyrimidin-2-yl)amino)phenyl)piperazine-1-carboxylate as yellow solid (0.15 g, 60%). ¹H NMR (400 MHz, DMSO-d₆): δ 11.9 (s, 1H), 9.07 (s, 1H), 8.64 (s, 1H), 7.57 (d, 2H), 7.38-7.34 (t, 1H), 7.28-7.24 (t, 1H), 7.17 (d, 1H), 6.85-6.78 (m, 2H), 6.45 (d, 1H), 5.45 (d, 2H), 4.18 (brs, 1H), 3.78 (d, 1H), 3.42-3.35 (m, 2H), 3.27 (s, 2H), 3.19 (s, 3H), 1.89 (s, 1H), 1.406 (s, 6H), 1.22 (d, 3H). LCMS m/z calcd for [M+H]⁺ 606.7. found 607.5.

### Step 2: (R)—N-methyl-N-(2-((2-((4-(3-methylpiperazin-1-yl)phenyl)amino)-7H-pyrrolo[2,3-d]pyrimidin-7-yl)methyl)phenyl)methanesulfonamide

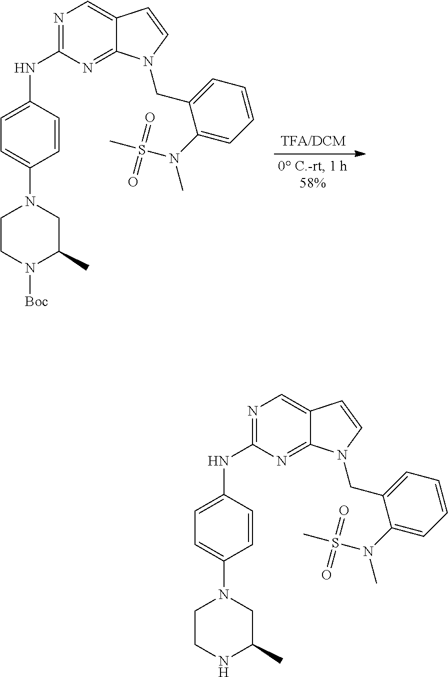 [SMILES: C[C@@H]1CN(c2ccc(Nc3ncc4ccn(Cc5ccccc5N(C)S(C)(=O)=O)c4n3)cc2)CCN1C(=O)OC(C)(C)C ; C[C@@H]1CN(c2ccc(Nc3ncc4ccn(Cc5ccccc5N(C)S(C)(=O)=O)c4n3)cc2)CCN1]

To a solution of (R)-tert-butyl 2-methyl-4-(4-((7-(2-(N-methylmethylsulfonamido)benzyl)-7H-pyrrolo[2,3-d]pyrimidin-2-yl)amino)phenyl)piperazine-1-carboxylate (0.15 g, 0.24 mmol) in DCM (10 mL) at 0° C., TFA (1.5 ml) was added and the mixture stirred at rt for 1 h while monitoring by TLC. After completion, the solvent was distilled off and the resulting crude product was dissolved in water (50 mL) and extracted with EtOAc (2×50 mL). The aqueous layer was basified with saturated NaHCO₃ solution (5 mL) and extracted with DCM (2×50 mL). The organic layer was dried over Na₂SO₄, filtered and concentrated to obtain pure (R)—N-methyl-N-(2-((2-((4-(3-methylpiperazin-1-yl)phenyl)amino)-7H-pyrrolo[2,3-d]pyrimidin-7-yl)methyl)phenyl)methanesulfonamide as off white solid (0.07 g, 58%). ¹H NMR (400 MHz, DMSO-d₆): δ 9.03 (s, 1H), 8.64 (s, 1H), 7.58-7.54 (m, 3H), 7.38-7.34 (m, 1H), 7.28-7.26 (m, 1H), 7.16 (s, 1H), 6.86-6.73 (m, 3H), 6.45 (d, 1H), 5.44 (d, 2H), 3.37-3.35 (m, 2H), 3.16 (s, 3H), 3.11 (s, 3H), 2.92-2.79 (m, 3H), 2.11 (m, 1H), 1.00 (d, 3H). LCMS m/z calcd for [M+H]⁺ 506.6 and found 506.4, HPLC purity 98.22%.

### Example 471

### N-Methyl-N-(3-{2-[4-((S)-3-methyl-piperazin-1-yl)-phenylamino]-pyrrolo[2,3-d]pyrimidin-7-ylmethyl}-pyridin-2-yl)-methanesulfonamide

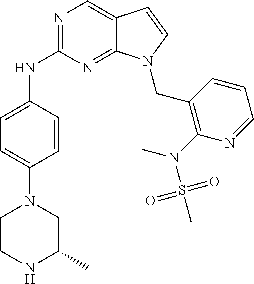 [SMILES: C[C@H]1CN(c2ccc(Nc3ncc4ccn(Cc5cccnc5N(C)S(C)(=O)=O)c4n3)cc2)CCN1]

### Step 1: (S)-tert-butyl 2-methyl-4-(4-((7-((2-(N-methylmethylsulfonamido)pyridin-3-yl)methyl)-7H-pyrrolo[2,3-d]pyrimidin-2-yl)amino)phenyl)piperazine-1-carboxylate

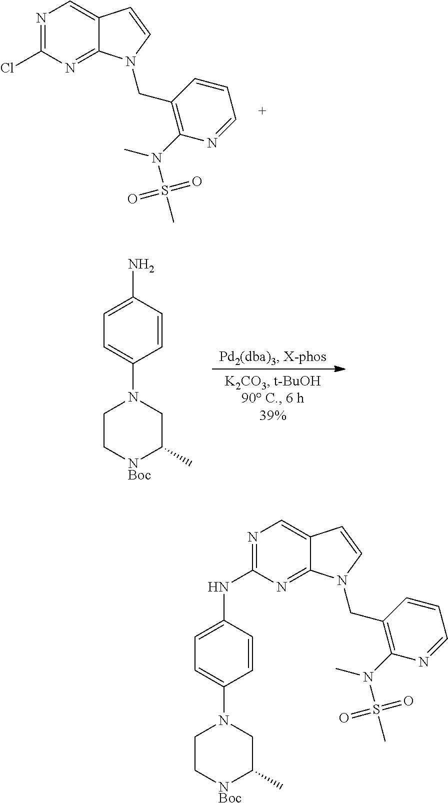 [SMILES: CN(c1ncccc1Cn1ccc2cnc(Cl)nc21)S(C)(=O)=O ; C[C@H]1CN(c2ccc(N)cc2)CCN1C(=O)OC(C)(C)C ; C[C@H]1CN(c2ccc(Nc3ncc4ccn(Cc5cccnc5N(C)S(C)(=O)=O)c4n3)cc2)CCN1C(=O)OC(C)(C)C]

A stirred mixture of N-(2-((2-chloro-7H-pyrrolo[2,3-d]pyrimidin-7-yl)methyl)phenyl)-N-methylmethanesulfonamide (1.0 g, 2.84 mmol, 1.0 eq), (S)-tert-butyl 4-(4-aminophenyl)-2-methylpiperazine-1-carboxylate (0.743 g, 2.56 mmol, 0.9 eq) and K₂CO₃ (1.17 g, 3.0 eq) in t-butanol (10.0 mL) in a vial was degassed using an argon balloon for 10 min. X-Phos (0.135 g, 0.1 eq) and Pd₂(dba)₃ (0.130 g, 0.05 eq) were added to this mixture and the mixture continued degassing for another 10 min. The vial was closed and the contents heated at 90° C. for 6 h. After completion of the starting material, the mixture was quenched with water and extracted with EtOAc (2×150 mL). The organic layer was dried over anhydrous Na₂SO₄, filtered and concentrated. The resulting residue was purified by Combiflash® chromatography using EtOAc in hexane as the eluent. The desired product eluted at 65% EtOAc in hexane. The fractions with product were concentrated to obtain (S)-tert-butyl 2-methyl-4-(4-((7-((2-(N-methylmethylsulfonamido)pyridin-3-yl)methyl)-7H-pyrrolo[2,3-d]pyrimidin-2-yl)amino)phenyl)piperazine-1-carboxylate as brown solid (0.61 g, 39%). ¹H NMR (400 MHz, DMSO-d₆): δ 9.155 (s, 1H), 8.675 (s, 1H), 8.469-8.460 (d, 1H), 7.570-7.548 (d, 2H), 7.393-7.362 (t, 1H), 7.289-7.246 (m, 2H), 6.822-6.800 (d, 2H), 6.495-6.487 (d, 1H), 5.483 (s, 2H), 4.200 (bs, 1H), 4.052-3.999 (m, 1H), 3.812-3.3.779 (d, 1H), 3.440-3.414 (d, 1H), 3.329 (s, 3H), 3.169 (s, 3H), 2.689-2.667 (d, 1H), 2.5 (s, merged with solvent peak, 1H), 1.987 (s, 1H), 1.420 (s, 9H), 1.227-1.211 (d, 3H). LCMS calculated for (M) 606.74 and found (M+H) 607.09. LCMS showed 96.85% purity.

### Step 2: (S)—N-methyl-N-(3-((2-((4-(3-methylpiperazin-1-yl)phenyl)amino)-7H-pyrrolo[2,3-d]pyrimidin-7-yl)methyl)pyridin-2-yl)methanesulfonamide

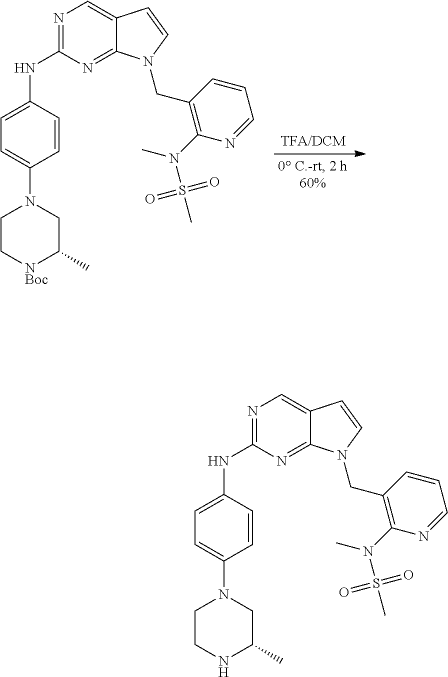 [SMILES: C[C@H]1CN(c2ccc(Nc3ncc4ccn(Cc5cccnc5N(C)S(C)(=O)=O)c4n3)cc2)CCN1C(=O)OC(C)(C)C ; C[C@H]1CN(c2ccc(Nc3ncc4ccn(Cc5cccnc5N(C)S(C)(=O)=O)c4n3)cc2)CCN1]

To a stirred solution of (S)-tert-butyl 2-methyl-4-(4-((7-((2-(N-methylmethylsulfonamido)pyridin-3-yl)methyl)-7H-pyrrolo[2,3-d]pyrimidin-2-yl)amino)phenyl)piperazine-1-carboxylate (0.6 g, 1.0 eq) in DCM (10.0 mL) in a two necked round bottomed flask under nitrogen atmosphere at 0° C. was added TFA (4.0 mL) and the mixture was stirred from 0° C. to rt for 2 h. After completion of the starting material, the solvent was distilled off and the resulting the crude product was dissolved in water (50 mL) and extracted with EtOAc (2×50 mL). The aqueous layer was basified with 10% NaOH and extracted with DCM (2×50 mL). The organic layer was dried over Na₂SO₄, filtered, concentrated and further washed with pentane and ether to obtain the pure (S)—N-methyl-N-(3-((2-((4-(3-methylpiperazin-1-yl)phenyl)amino)-7H-pyrrolo[2,3-d]pyrimidin-7-yl)methyl)pyridin-2-yl)methanesulfonamide as off white solid (0.5 g, 60%). ¹H NMR (400 MHz, DMSO-d₆): δ 9.095 (s, 1H), 8.665 (s, 1H), 8.479-8.460 (d, 1H), 7.539-7.517 (d, 2H), 7.393-7.362 (m, 1H), 7.307-7.289 (d, 1H), 7.242-7.234 (d, 1H), 6.799-6.778 (d, 2H), 6.489-6.480 (d, 1H), 5.476 (s, 2H), 3.386-3.376 (d, 2H), 3.168 (s, 6H), 2.945-2.916 (d, 1H), 2.810-2.759 (t, 2H), 2.452-2.424 (d, 1H), 2.125-2.073 (t, 1H), 1.015-0.999 (d, 3H). LCMS calculated for (M) 506.62 and found (M+H) 507.18. LCMS showed 98.96% purity.

### Example 585

### (S)—N-methyl-N-(3-((2-((5-(3-methylpiperazin-1-yl)pyridin-2-yl)amino)-7H-pyrrolo[2,3-d]pyrimidin-7-yl)methyl)pyridin-2-yl)methanesulfonamide

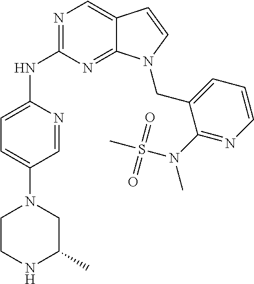 [SMILES: C[C@H]1CN(c2ccc(Nc3ncc4ccn(Cc5cccnc5N(C)S(C)(=O)=O)c4n3)nc2)CCN1]

### Step 1: (S)-tert-butyl 2-methyl-4-(6-((7-((2-(N-methylmethylsulfonamido)pyridin-3-yl)methyl)-7H-pyrrolo[2,3-d]pyrimidin-2-yl)amino)pyridin-3-yl)piperazine-1-carboxylate

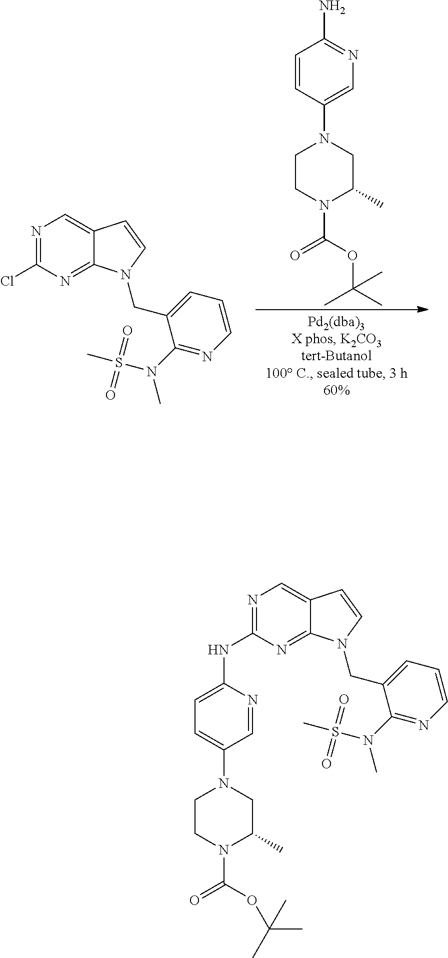 [SMILES: CN(c1ncccc1Cn1ccc2cnc(Cl)nc21)S(C)(=O)=O ; C[C@H]1CN(c2ccc(N)nc2)CCN1C(=O)OC(C)(C)C ; C[C@H]1CN(c2ccc(Nc3ncc4ccn(Cc5cccnc5N(C)S(C)(=O)=O)c4n3)nc2)CCN1C(=O)OC(C)(C)C]

A stirred mixture of N-(3-((2-chloro-7H-pyrrolo[2,3-d]pyrimidin-7-yl)methyl)pyridin-2-yl)-N-methylmethanesulfonamide (0.15 g, 0.42 mmol, 1.0 eq), (S)-tert-butyl 4-(6-aminopyridin-3-yl)-2-methylpiperazine-1-carboxylate (0.15 g, 0.42 mmol, 0.9 eq) and K₂CO₃ (0.17 g, 1.28 mmol, 3.0 eq) in t-butanol (5.0 mL) in a vial was degassed using an argon balloon for 10 min. X-Phos (0.020 g, 0.042 mmol, 0.1 eq) and Pd₂(dba)₃ (0.019 g, 0.021 mmol, 0.05 eq) were added to this mixture and the solution continued degassing for another 10 min. The vial was closed and the contents heated at 90° C. for 3 h. After completion of the starting material, the mixture was quenched with water (30 mL) and extracted with EtOAc (2×50 mL). The organic layer was dried over anhydrous Na₂SO₄, filtered and concentrated. The resulting residue was purified by flash chromatography (Biotage® Isolera™ purifier, 25 g column) using EtOAc in hexane as eluent. The desired product eluted at 60% EtOAc in hexane. The fractions with product were concentrated to obtain (S)-tert-butyl 2-methyl-4-(6-((7-((2-(N-methylmethylsulfonamido) pyridin-3-yl)methyl)-7H-pyrrolo[2,3-d]pyrimidin-2-yl)amino)pyridin-3-yl)piperazine-1-carboxylate (0.15 g, 60%); ¹H NMR (400 MHz, DMSO-d₆): δ 8.68 (s, 1H), 8.395 (d, J=2 Hz, 1H), 8.24 (d, J=4 Hz, 1H), 7.935 (d, J=2 Hz, 1H), 7.65 (s, 1H), 7.45 (d, J=4 Hz, 1H), 7.25-7.19 (m, 3H), 7.065 (d, J=2 Hz, 2H), 5.61 (s, 2H), 4.36 (s, 1H), 3.965 (d, 1H), 3.245 (d, 1H), 3.23 (s, 3H), 3.21 (d, 2H), 2.91 (s, 3H), 2.875 (d, 2H), 2.74-2.68 (m, 1H), 1.48 (s, 9H), 1.325 (d, 3H); LCMS m/z calcd for [M+H]⁺ 607.73. found 608.3.

### Step 2: (S)—N-methyl-N-(3-((2-((5-(3-methylpiperazin-1-yl)pyridin-2-yl)amino)-7H-pyrrolo[2,3-d]pyrimidin-7-yl)methyl)pyridin-2-yl)methanesulfonamide

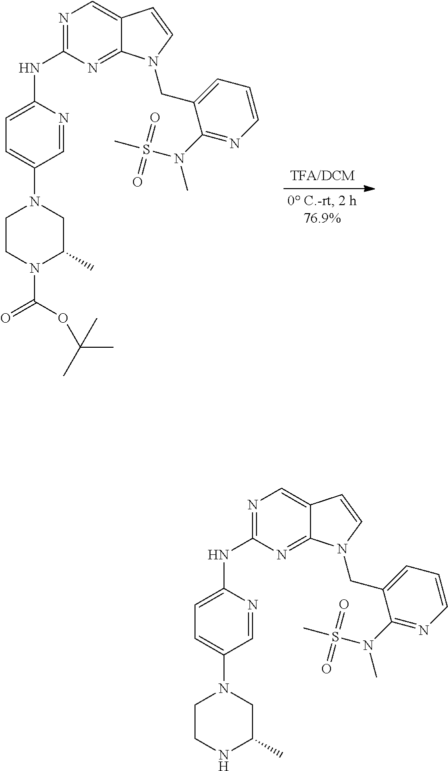 [SMILES: C[C@H]1CN(c2ccc(Nc3ncc4ccn(Cc5cccnc5N(C)S(C)(=O)=O)c4n3)nc2)CCN1C(=O)OC(C)(C)C ; C[C@H]1CN(c2ccc(Nc3ncc4ccn(Cc5cccnc5N(C)S(C)(=O)=O)c4n3)nc2)CCN1]

To a solution of (S)-tert-butyl 2-methyl-4-(6-((7-((2-(N-methylmethylsulfonamido)pyridin-3-yl)methyl)-7H-pyrrolo[2,3-d]pyrimidin-2-yl)amino)pyridin-3-yl)piperazine-1-carboxylate (0.19 g, 0.313 mmol) in DCM (10 mL) was added TFA (1.0 mL) at 0° C., and the mixture stirred at rt for 2 h. The mixture was evaporated under vacuum to remove the TFA, and the mixture was basified with saturated NaHCO₃ (5 mL), extracted with DCM (50 mL), dried over sodium sulfate, filtered and evaporated under reduced pressure to obtain (S)—N-methyl-N-(3-((2-((5-(3-methylpiperazin-1-yl)pyridin-2-yl)amino)-7H-pyrrolo[2,3-d]pyrimidin-7-yl)methyl)pyridin-2-yl)methanesulfonamide (0.122 g, 76.9% yield). ¹H NMR (400 MHz, DMSO-d₆): δ 9.02 (s, 1H), 8.71 (s, 1H), 8.44 (s, 1H), 8.02 (d, J=12 Hz, 1H), 7.91 (s, 1H), 7.36 (s, 2H), 7.28 (d, J=4.0 Hz, 1H), 7.25 (d, J=12 Hz, 1H), 6.51 (d, J=4.0 Hz, 1H), 5.50 (s, 2H), 3.4 (t, J=12 Hz, 3H), 3.14 (d, J=8.0 Hz, 6H), 2.96 (d, J=12 Hz, 1H), 2.82 (t, J=8.0 Hz, 2H), 2.18 (t, J=12 Hz, 1H), 1.02 (d, J=8.0 Hz, 3H) LCMS m/z calcd for [M+H]⁺ 507.61. found 508.3, HPLC purity 98.23%.

### Example 596

### N-(2-methoxyethyl)-N-(2-((2-((4-(piperazin-1-yl)phenyl)amino)-7H-pyrrolo[2,3-d]pyrimidin-7-yl)methyl)phenyl)methanesulfonamide

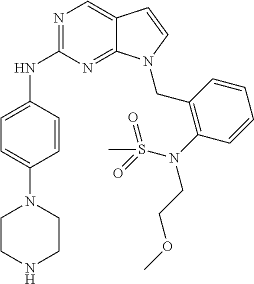 [SMILES: COCCN(c1ccccc1Cn1ccc2cnc(Nc3ccc(N4CCNCC4)cc3)nc21)S(C)(=O)=O]

### Step 1: N-(2-((2-chloro-7H-pyrrolo[2,3-d]pyrimidin-7-yl)methyl)phenyl)-N-(2-methoxyethyl)methanesulfonamide

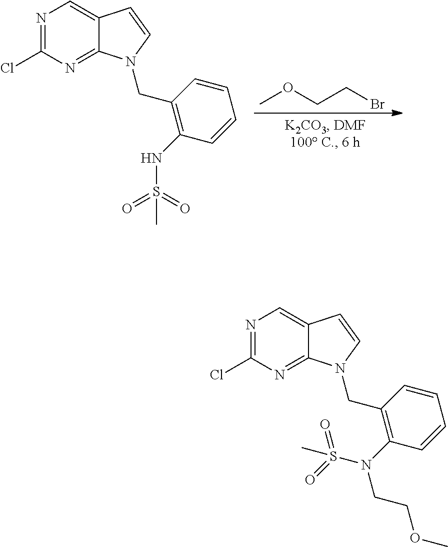

To a solution of N-(2-((2-chloro-7H-pyrrolo[2,3-d]pyrimidin-7-yl)methyl)phenyl)methanesulfonamide (150 mg, 0.466 mmol) in DMF (10 mL) was added potassium carbonate (369 mg, 2.676 mmol). The mixture was stirred for 30 min at rt. 1-Bromo-2-methoxyethane (97 mg, 0.669 mmol) was then added to the mixture and the mixture was stirred for 2 h at 100° C. Progress of the reaction was followed by TLC (50% ethyl acetate/hexane). After completion of the reaction, the mixture was diluted with ethyl acetate (50 mL) and washed with ice water (50 mL×2), followed by brine (20 mL). The organic layer was dried over Na₂SO₄, filtered and evaporated to give crude product, which was purified by Biotage® Isolera™ by using 40% ethyl acetate/hexane to give N-(2-((2-chloro-7H-pyrrolo[2,3-d]pyrimidin-7-yl)methyl)phenyl)-N-(2-methoxyethyl)methanesulfonamide (Yield: 150 mg, 86%). ¹H NMR (400 MHz, DMSO-d₆): 8.96 (s, 1H), 7.57-7.53 (t, 2H, J=4 Hz), 7.40-7.36 (t, 1H, J=8 Hz), 7.26-7.22 (t, 1H, J=8 Hz), 6.76-6.75 (d, 1H, J=4 Hz), 6.58-6.56 (d, 1H, J=8 Hz), 5.66-5.47 (dd, 2H), 3.96-3.91 (m, 1H), 3.73-3.69 (m, 1H), 3.45-3.40 (m, 2H), 3.25 (s, 3H), 3.12 (s, 3H).

### Step 2: tert-butyl 4-(4-((7-(2-(N-(2-methoxyethyl)methylsulfonamido)benzyl)-7H-pyrrolo[2,3-d]pyrimidin-2-yl)amino)phenyl)piperazine-1-carboxylate

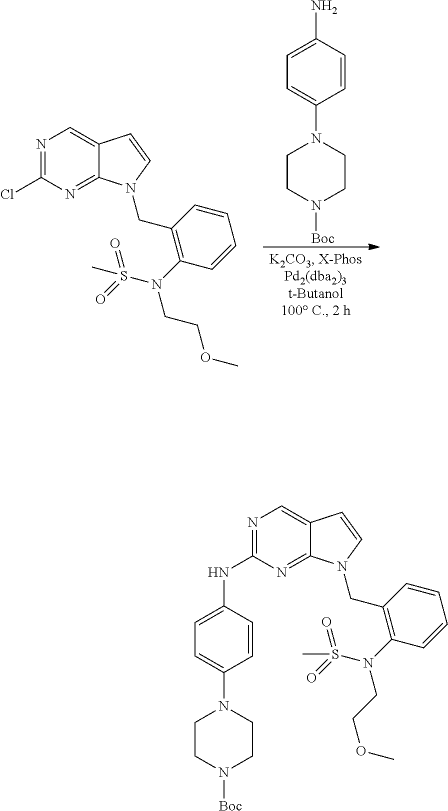 [SMILES: COCCN(c1ccccc1Cn1ccc2cnc(Cl)nc21)S(C)(=O)=O ; CC(C)(C)OC(=O)N1CCN(c2ccc(N)cc2)CC1 ; COCCN(c1ccccc1Cn1ccc2cnc(Nc3ccc(N4CCN(C(=O)OC(C)(C)C)CC4)cc3)nc21)S(C)(=O)=O]

A solution of N-(2-((2-chloro-7H-pyrrolo[2,3-d]pyrimidin-7-yl)methyl)phenyl)-N-(2-methoxyethyl)methanesulfonamide (150 mg, 0.379 mmol), tert-butyl 4-(4-aminophenyl)piperazine-1-carboxylate (84 mg, 0.303 mmol), potassium carbonate (157 mg, 1.137 mmol) and X-Phos (18 mg, 0.0379 mmol) in tert-Butanol (10 mL) under argon atmosphere was purged with argon for 30 min. Tris(dibenzylideneacetone)dipalladium (17 mg, 0.0189 mmol) was then added to the mixture and the mixture was stirred for 2 h at 100° C. Progress of the reaction was followed by TLC (30% ethyl acetate/hexane). After completion of the reaction, the mixture was diluted with ethyl acetate (75 mL), washed with water (20 mL), followed by brine (20 mL), dried over Na₂SO₄, filtered and evaporated to give crude product. The resulting residue was purified by Biotage® Isolera™ using 2% MeOH/DCM to give tert-butyl 4-(4-((7-(2-(N-(2-methoxyethyl) methylsulfonamido)benzyl)-7H-pyrrolo[2,3-d]pyrimidin-2-yl)amino)phenyl)piperazine-1-carboxylate (Yield: 50 mg, 21%).

### Step 3: N-(2-methoxyethyl)-N-(2-((2-((4-(piperazin-1-yl)phenyl)amino)-7H-pyrrolo[2,3-d]pyrimidin-7-yl)methyl)phenyl)methanesulfonamide

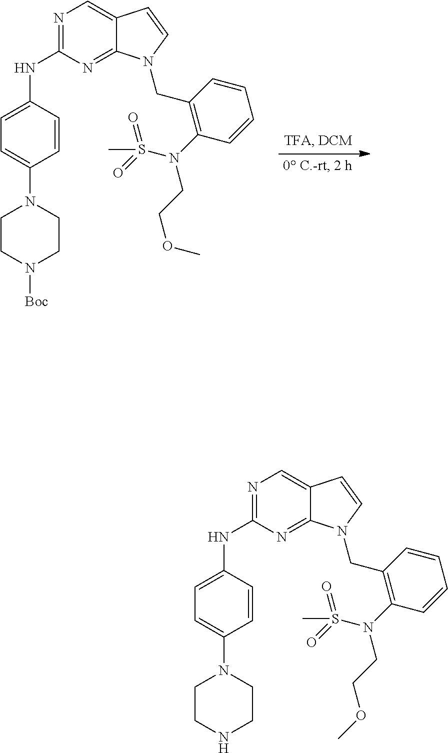 [SMILES: COCCN(c1ccccc1Cn1ccc2cnc(Nc3ccc(N4CCN(C(=O)OC(C)(C)C)CC4)cc3)nc21)S(C)(=O)=O ; COCCN(c1ccccc1Cn1ccc2cnc(Nc3ccc(N4CCNCC4)cc3)nc21)S(C)(=O)=O]

A solution of tert-butyl 4-(4-((7-(2-(N-(2-methoxyethyl)methylsulfonamido) benzyl)-7H-pyrrolo[2,3-d]pyrimidin-2-yl)amino)phenyl)piperazine-1-carboxylate (50 mg, 0.787 mmol) in dichloromethane (5 mL) under argon atmosphere was cooled to 0° C. Trifluoroacetic acid (30 μL, 0.393 mmol) was added to the mixture and the mixture stirred for 2 h at rt. Progress of the reaction was followed by TLC (10% MeOH/DCM). After completion of the reaction, the solvent was evaporated under reduced pressure to give crude residue, which was dissolved in water (10 mL) and extracted with ethyl acetate (20 m). The aqueous layer was basified with saturated NaHCO₃ solution and extracted with DCM (20 mL×2). The combined DCM layers were dried over Na₂SO₄, filtered and evaporated to give crude product. The resulting crude product was purified by Biotage® Isolera™ by using 7% MeOH/DCM to give N-(2-methoxyethyl)-N-(2-((2-((4-(piperazin-1-yl)phenyl)amino)-7H-pyrrolo[2,3-d]pyrimidin-7-yl)methyl)phenyl)methanesulfonamide (Yield: 12 mg, 29%). ¹H NMR (400 MHz, DMSO-d₆): δ 9.09 (s, 1H), 8.67 (s, 1H), 7.56-7.54 (d, 2H), 7.39-7.35 (t, 1H, J=8 Hz), 7.27-7.23 (t, 1H, J=8 Hz), 7.04-7.03 (d, 1H, J=4 Hz), 6.77-6.74 (d, 2H, J=12 Hz), 6.64-6.62 (d, 1H, J=8 Hz), 6.47-6.46 (d, 1H, J=4 Hz), 5.48 (s, 2H), 3.89-3.85 (d, 1H, J=16 Hz), 3.72-3.68 (d, 1H, J=16 Hz), 3.65-3.35 (d, 2H, J=14 Hz), 3.19 (s, 3H), 3.12 (s, 3H), 2.98 (s, 4H), 2.97 (s, 4H). [M+1] 536.3, Purity: 99.19%.

### Example 633

### N-(3-((2-((1-(2-(dimethylamino)ethyl)-2-oxoindolin-5-yl)amino)-7H-pyrrolo[2,3-d]pyrimidin-7-yl)methyl)pyridin-2-yl)-N-methylmethanesulfonamide

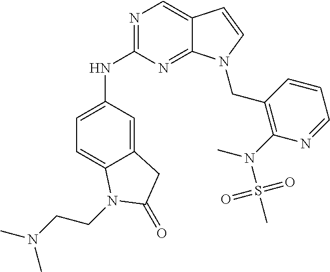 [SMILES: CN(C)CCN1C(=O)Cc2cc(Nc3ncc4ccn(Cc5cccnc5N(C)S(C)(=O)=O)c4n3)ccc21]

### Step 1: 2-(5-((7-((2-(N-methylmethylsulfonamido)pyridin-3-yl)methyl)-7H-pyrrolo[2,3-d]pyrimidin-2-yl)amino)-2-oxoindolin-1-yl)ethylmethanesulfonate

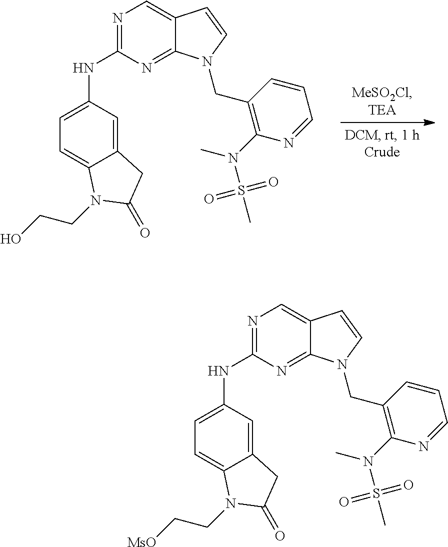 [SMILES: CN(c1ncccc1Cn1ccc2cnc(Nc3ccc4c(c3)CC(=O)N4CCO)nc21)S(C)(=O)=O ; CN(c1ncccc1Cn1ccc2cnc(Nc3ccc4c(c3)CC(=O)N4CCOS(C)(=O)=O)nc21)S(C)(=O)=O]

To a solution of N-(3-((2-((1-(2-hydroxyethyl)-2-oxoindolin-5-yl)amino)-7H-pyrrolo[2,3-d]pyrimidin-7-yl)methyl)pyridin-2-yl)-N-methylmethanesulfonamide (0.15 g, 1.0 eq) in DCM (10 mL) at rt, methane sulfonyl chloride (0.183 mL, 8.0 eq) and TEA (0.82 mL, 20.0 eq) were added and the mixture was stirred for 1 h. After TLC showed completion of starting material, the mixture was quenched with water (50 mL) and extracted with DCM (2×50 mL). The organic layer was dried over anhydrous Na₂SO₄, filtered and concentrated to obtain 2-(5-((7-((2-(N-methylmethylsulfonamido)pyridin-3-yl)methyl)-7H-pyrrolo[2,3-d]pyrimidin-2-yl)amino)-2-oxoindolin-1-yl)ethylmethanesulfonate as yellow liquid (0.18 g, crude). LCMS calculated for (M) 585.15 and found (M+H) 586.39. LCMS showed 94.40% purity.

### Step 2: 2-(5-((7-((2-(N-methylmethylsulfonamido)pyridin-3-yl)methyl)-7H-pyrrolo[2,3-d]pyrimidin-2-yl)amino)-2-oxoindolin-1-yl)ethylmethanesulfonate

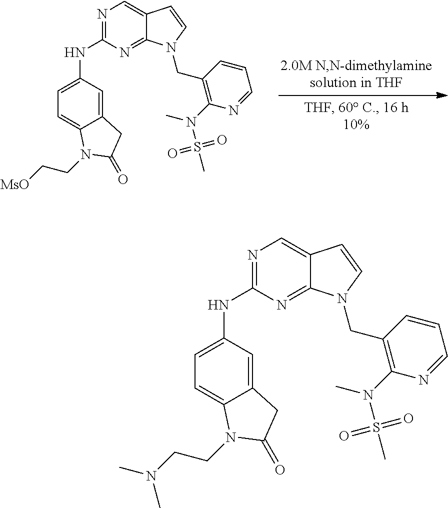 [SMILES: CN(c1ncccc1Cn1ccc2cnc(Nc3ccc4c(c3)CC(=O)N4CCOS(C)(=O)=O)nc21)S(C)(=O)=O ; CN(C)CCN1C(=O)Cc2cc(Nc3ncc4ccn(Cc5cccnc5N(C)S(C)(=O)=O)c4n3)ccc21]

To a solution of 2-(5-((7-((2-(N-methylmethylsulfonamido)pyridin-3-yl) methyl)-7H-pyrrolo[2,3-d]pyrimidin-2-yl)amino)-2-oxoindolin-1-yl)ethylmethanesulfonate (0.175 g, 1.0 eq) in THF (10 mL) in a sealed tube at rt, a 2.0 M solution of N,N-dimethylamine in THF (5 mL) was added and the mixture was stirred at 60° C. for 16 h. After TLC showed completion of starting material, the mixture was diluted with ethyl acetate (50 mL) and washed with water (2×50 mL). The organic layer was dried over anhydrous Na₂SO₄, filtered and concentrated. The crude was purified using Combiflash™ chromatography using methanol in DCM as eluent. The product eluted at 7% methanol in DCM. Fractions with product were distilled off under reduced pressure to obtain 2-(5-((7-((2-(N-methylmethylsulfonamido)pyridin-3-yl)methyl)-7H-pyrrolo[2,3-d]pyrimidin-2-yl)amino)-2-oxoindolin-1-yl)ethylmethanesulfonate as off-white solid (0.016 g, 10%). ¹H NMR (400 MHz, DMSO-d₆): δ 9.30 (s, 1H), 8.70 (s, 1H), 8.46 (s, 1H), 7.67 (s, 1H), 7.599-7.579 (d, 1H), 7.376-7.364 (d, 1H), 7.250 (s, 2H), 6.856-6.835 (d, 1H), 6.509 (s, 1H), 5.513 (s, 2H), 3.707 (brs, 2H), 3.478 (s, 2H), 3.172-3.155 (d, 8H), 2.185 (s, 6H). LCMS calculated for (M) 534.22 and found (M+H) 535.50. LCMS showed 98.51% purity.

### Example 663

### N-(3-((2-((4-((S)-4-((S)-2-Hydroxypropyl)-3-methylpiperazin-1-yl)phenyl)amino)-7H-pyrrolo[2,3-d]pyrimidin-7-yl)methyl)pyridin-2-yl)-N-methylmethanesulfonamide

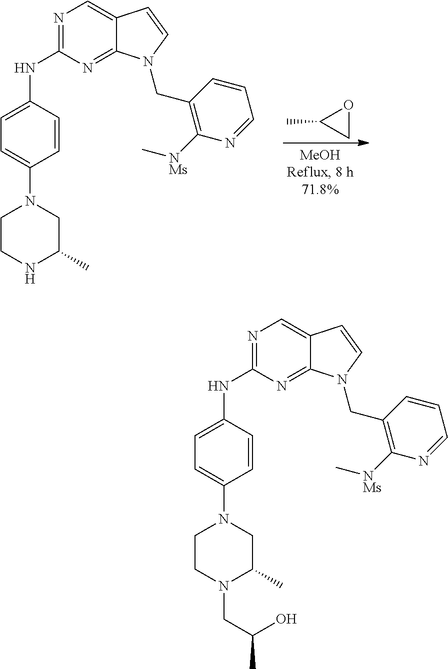

To a stirred solution of (S)—N-methyl-N-(3-((2-((4-(3-methylpiperazin-1-yl)phenyl)amino)-7H-pyrrolo[2,3-d]pyrimidin-7-yl)methyl)pyridin-2-yl)methanesulfonamide (150 mg, 0.00029 mol, 1.0 eq) in MeOH in a vial under nitrogen atmosphere, (S)-2-methyloxirane (25 mg, 0.00044 mol, 1.5 eq) was added and the mixture was refluxed for 8 h while monitoring by TLC. After completion of the starting material, the solvent was distilled off of the mixture to obtain crude residue. This crude was purified by Combiflash™ chromatography on silica gel (230-400#) using MeOH in DCM as eluent. The desired product eluted at 2.8% MeOH in DCM and concentration of the fractions provided N-(3-((2-((4-((S)-4-((S)-2-Hydroxypropyl)-3-methylpiperazin-1-yl)phenyl)amino)-7H-pyrrolo[2,3-d]pyrimidin-7-yl)methyl)pyridin-2-yl)-N-methylmethanesulfonamide as off-white solid (120 mg, 71.8%). ¹H NMR (400 MHz, DMSO-d₆): δ 9.108 (s, 1H), 8.667 (s, 1H), 8.466 (d, 1H), 7.535 (d, 1H), 7.378 (m, 1H), 7.292 (d, 1H), 7.241 (d, 1H), 6.797 (d, 2H), 6.487 (d, 1H), 5.477 (s, 2H), 4.162 (d, 1H), 3.744 (brs, 1H), 3.215 (m, 3H), 3.170 (d, 6H), 2.959 (t, 1H), 2.749 (m, 1H), 2.395 (m, 4H), 2.059 (d, 1H), 1.049 (d, 6H). LCMS calculated for (M) 564.3 and found (M+H) 565.30, LCMS showed 99.02% purity.

### Example 679

### N-(3-((2-((4-((S)-4-((R)-2-hydroxypropyl)-3-methylpiperazin-1-yl)phenyl)amino)-7H-pyrrolo[2,3-d]pyrimidin-7-yl)methyl)pyridin-2-yl)-N-methylmethanesulfonamide

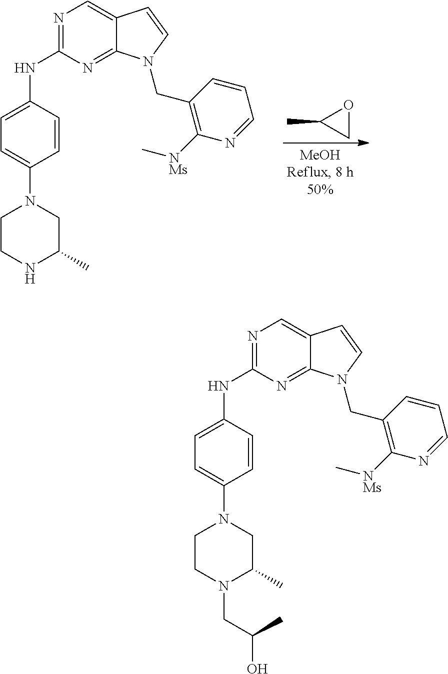

To a stirred solution of (S)—N-methyl-N-(3-((2-((4-(3-methylpiperazin-1-yl)phenyl)amino)-7H-pyrrolo[2,3-d]pyrimidin-7-yl)methyl)pyridin-2-yl)methanesulfonamide (180 mg, 0.0003 mol, 1.0 eq) in MeOH in a vial under nitrogen atmosphere, (R)-2-methyloxirane (30 mg, 0.0005 mol, 1.5 eq) was added and the mixture refluxed for 8 h while monitoring by TLC. After completion of starting material, the solvent was distilled off to obtain crude residue. This crude material was purified with Combiflash™ chromatography on silica gel with MeOH in DCM as eluent. The product eluted at 3.1% MeOH in DCM and concentration of the pure fractions afforded 3-((2-((4-((S)-4-((R)-2-hydroxypropyl)-3-methylpiperazin-1-yl)phenyl)amino)-7H-pyrrolo[2,3-d]pyrimidin-7-yl)methyl)pyridin-2-yl)-N-methylmethanesulfonamide as off white solid (100 mg, 50%). ¹H NMR (400 MHz, DMSO-d₆): δ 9.107 (s, 1H), 8.667 (s, 1H), 8.469 (s, 1H), 7.533 (d, 2H), 7.373 (t, 1H), 7.294 (d, 1H), 7.240 (d, 1H), 6.794 (d, 2H), 6.485 (s, 1H), 5.477 (s, 2H), 4.368 (s, 1H), 3.750 (brs, 1H), 3.266 (m, 2H), 3.169 (s, 6H), 2.921 (d, 1H), 2.690 (m, 1H), 2.403 (m, 4H), 2.147 (d, 1H), 1.060 (d, 6H).

### Example 695

### N-(2-((2-((4-((S)-4-((S)-2-hydroxypropyl)-3-methylpiperazin-1-yl)phenyl)amino)-7H-pyrrolo[2,3-d]pyrimidin-7-yl)methyl)phenyl)-N-methylmethanesulfonamide

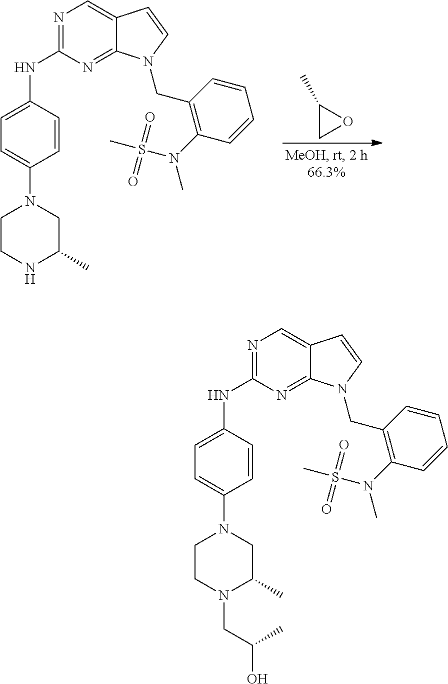

To a solution of (S)—N-methyl-N-(2-((2-((4-(3-methylpiperazin-1-yl)phenyl)amino)-7H-pyrrolo[2,3-d]pyrimidin-7-yl)methyl)phenyl)methanesulfonamide (0.1 g, 1.0 eq) in methanol (10 mL) under nitrogen atmosphere, (S)-2-methyloxirane (1.5 eq) was added and the mixture stirred at rt for 2 h. After completion of the starting material on TLC, the mixture was distilled to provide crude material. The resulting residue was purified with Combiflash® chromatography over silica gel (230-400#) with MeOH in DCM as eluent. The desired product eluted at 2% MeOH in DCM. Concentration of the pure fractions provided N-(2-((2-((4-((S)-4-((S)-2-hydroxypropyl)-3-methylpiperazin-1-yl)phenyl)amino)-7H-pyrrolo[2,3-d]pyrimidin-7-yl)methyl)phenyl)-N-methylmethanesulfonamide as off white solid (0.07 g, 66.3%). ¹H NMR (400 MHz, DMSO-d₆): δ 9.075 (s, 1H), 8.659 (s, 1H), 7.584 (t, 3H), 7.38 (t, 1H), 7.28 (t, 1H), 7.184 (d, 1H), 6.832 (dd, 3H), 6.469 (d, 1H), 5.486 (d, 2H), 4.154 (s, 1H), 3.743 (brs, 1H), 3.145 (d, 6H), 2.945 (d, 1H), 2.738 (t, 1H), 2.587 (t, 1H), 2.443 (m, 3H), 2.072 (t, 1H), 1.046 (s, 6H). LCMS calculated for (M) 563.23 and found (M+H) 564.28, LCMS showed 99.42% purity.

### Example 759

### N-methyl-N-(4-methyl-2-((2-((4-(piperazin-1-yl)phenyl)amino)-7H-pyrrolo[2,3-d]pyrimidin-7-yl)methyl)phenyl)methanesulfonamide

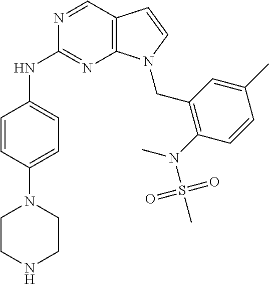 [SMILES: Cc1ccc(N(C)S(C)(=O)=O)c(Cn2ccc3cnc(Nc4ccc(N5CCNCC5)cc4)nc32)c1]

### Step 1: Methyl 5-methyl-2-nitrobenzoate

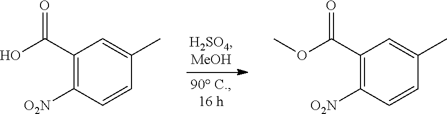

A solution of 5-methyl-2-nitrobenzoic acid (8 g, 44.198 mmol) in methanol (80 mL) was cooled to 0° C. Sulfuric acid (5 mL) was added to the mixture drop wise. The mixture was heated to 90° C. and stirred for 16 h. Progress of the reaction was followed by TLC (50% ethyl acetate/hexane). After completion of the reaction, excess methanol was evaporated and the residue was poured into water (150 mL) and extracted with ethyl acetate (150 mL×2). The combined organic layers were washed with saturated NaHCO₃ solution (50 mL×2), followed by brine (20 mL). The organic layer was dried over Na₂SO₄, filtered and evaporated to give methyl 5-methyl-2-nitrobenzoate (Yield: 8 g, 93%). ¹H NMR (400 MHz, CDCl₃): δ 7.86-7.84 (d, 1H, J=8 Hz), 7.48 (s, 1H), 7.40-7.38 (d, 1H, J=8 Hz), 3.91 (s, 3H), 3.47 (s, 3H).

### Step 2: Methyl 2-amino-5-methylbenzoate

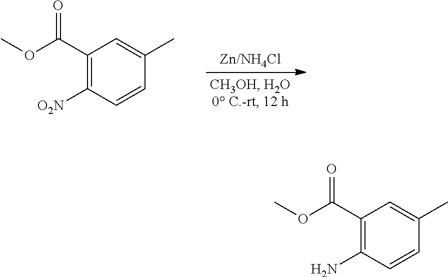

A solution of methyl 5-methyl-2-nitrobenzoate (8 g, 41.025 mmol) in methanol (80 mL) and water (10 mL) was cooled to 0° C. Zinc powder (13.33 g, 205.128 mmol) was then added, followed by NH₄Cl (17.6 g, 328.2 mmol). The mixture was stirred for 12 h at rt. Progress of the reaction was followed by TLC (20% ethyl acetate/hexane). After completion of the reaction, the mixture was filtered and the filtrate was evaporated to provide crude residue. The crude reside was basified with saturated NaHCO₃ solution and extracted with DCM (2×20 mL). The combined organic layers were dried over Na₂SO₄, filtered and evaporated to give methyl 2-amino-5-methylbenzoate (Yield: 4 g, 60%). ¹H NMR (400 MHz, CDCl₃): δ 7.65 (s, 1H), 7.25 (s, 1H), 7.10-7.07 (q, 1H, J=8 Hz), 6.58 (d, 1H), 3.86 (s, 3H), 2.22 (s, 3H).

### Step 3: Methyl 5-methyl-2-(methylsulfonamido) benzoate

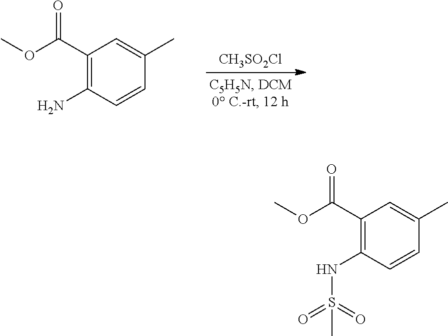

A solution of methyl 2-amino-5-methylbenzoate (2.76 g, 16.727 mmol) in DCM (30 mL) and pyridine (6.6 mL, 83.636 mmol) was cooled to 0° C. Methane sulfonyl chloride (2.88 g, 1.95 mL, 25.09 mmol) was then added to the mixture drop-wise. The mixture was stirred for 5 h at rt. Progress of the reaction was followed by TLC (25% ethyl acetate/hexane). After completion of the reaction, the mixture was diluted with water (100 mL) and extracted with DCM (50 mL×2). The combined organic layers were washed with saturated NH₄Cl solution (50 mL), followed brine (20 mL). The mixture was then dried over Na₂SO₄, filtered and evaporated to provide crude product. The resulting residue was purified by Biotage® Isolera™ using 15% ethyl acetate/hexane to provide methyl 5-methyl-2-(methylsulfonamido)benzoate (Yield: 3.36 g, 84%). ¹H NMR (400 MHz, CDCl₃): δ 10.20 (s, 1H), 7.83 (s, 1H), 7.63-7.61 (d, 1H, J=8 Hz), 7.37-7.34 (d, 1H, J=12 Hz), 3.92 (s, 3H), 3.0 (s, 3H), 2.33 (s, 3H).

### Step 4: Methyl 5-methyl-2-(N-methylmethylsulfonamido) benzoate

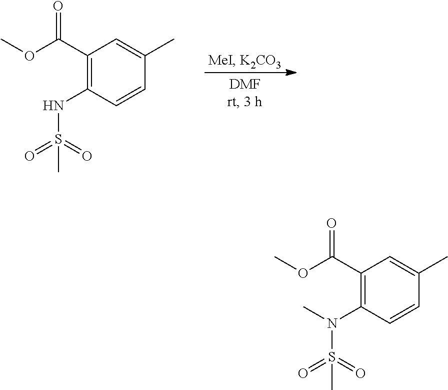

To a solution of methyl 5-methyl-2-(methylsulfonamido) benzoate (3.36 g, 13.884 mmol) in DMF (30 mL) was added potassium carbonate (9.57 g, 69.42 mmol) and the solution stirred for 30 min at rt. Methyl iodide (2.95 g, 1.32 mL, 20.826 mmol) was then added to the mixture drop-wise. The mixture was stirred for 2 h at rt. Progress of the reaction was followed by TLC (30% ethyl acetate/hexane). After completion of the reaction, the mixture was diluted with water (of) and extracted with ethyl acetate (100 mL×2). The combined organic layers were washed with water (100 mL×2), followed by brine (50 mL). The organic layer was dried over Na₂SO₄, filtered and evaporated to provide crude product. The resulting residue was purified by Biotage® Isolera™ using 15% ethyl acetate/hexane to give methyl 5-methyl-2-(N-methylmethylsulfonamido) benzoate (Yield: 3.2 g, 91.4%). ¹H NMR (400 MHz, CDCl₃): δ 7.70 (s, 1H), 7.34-7.33 (d, 2H, J=4 Hz), 3.96 (s, 3H), 3.28 (s, 2H), 2.94 (s, 3H), 2.38 (s, 3H).

### Step 5: N-(2-(hydroxymethyl)-4-methylphenyl)-N-methylmethanesulfonamide

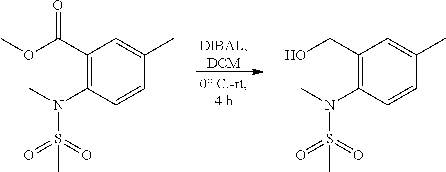

A solution of methyl 5-methyl-2-(N-methylmethylsulfonamido)benzoate (3.8 g, 14.785 mmol) in DCM (30 mL) under nitrogen atmosphere was cooled to 0° C. A solution of diisobutylaluminiumhydride in toluene (25 mL) was then added to the mixture drop wise. The mixture was stirred for 4 h at rt. Progress of the reaction was followed by TLC (40% ethyl acetate/hexane). After completion of the reaction, the mixture was cooled to 0° C. and quenched with saturated Na₂SO₄ solution. The solid was filtered and the filtrate was evaporated and dried to give N-(2-(hydroxymethyl)-4-methylphenyl)-N-methylmethanesulfonamide (Yield: 3 g, 89%). ¹H NMR (400 MHz, CDCl₃): δ 7.40 (s, 1H), 7.159-7.157 (d, 2H, J=8 Hz), 4.8 (brs, 2H), 3.25 (s, 3H), 2.96 (s, 3H), 2.37 (s, 3H).

### Step 6: N-(2-(chloromethyl)-4-methylphenyl)-N-methylmethanesulfonamide

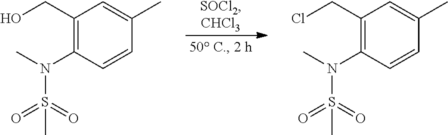

To a solution of N-(2-(hydroxymethyl)-4-methylphenyl)-N-methylmethanesulfonamide (3 g, 13.10 mmol) and chloroform (30 mL) was added thionyl chloride (1.87 g, 1.8 mL, 15.72 mmol). The mixture was heated to 50° C. for 2 h. Progress of the reaction was followed by TLC (40% ethyl acetate/hexane). After completion of the reaction, the mixture was poured onto ice/water (50 mL) and extracted with DCM (50 mL×2). The combined organic layers were washed with water (50 mL×2), followed by brine (50 mL), dried over Na₂SO₄, filter and evaporated to give N-(2-(chloromethyl)-4-methylphenyl)-N-methylmethanesulfonamide (Yield: 3 g, 94%). ¹H NMR (400 MHz, CDCl₃): δ 7.37 (s, 1H), 7.17 (s, 2H), 5.0 (brs, 1H), 4.5 (brs, 1H), 3.29 (s, 3H), 2.95 (s, 3H), 2.37 (s, 3H).

### Step 7: N-(2-((2-chloro-7H-pyrrolo[2,3-d]pyrimidin-7-yl)methyl)-4-methylphenyl)-N-methylmethanesulfonamide

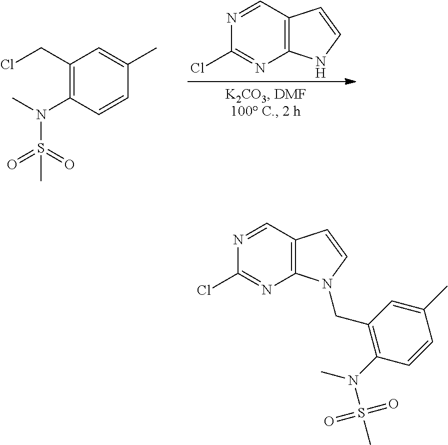 [SMILES: Cc1ccc(N(C)S(C)(=O)=O)c(CCl)c1 ; Clc1ncc2cc[nH]c2n1 ; Cc1ccc(N(C)S(C)(=O)=O)c(Cn2ccc3cnc(Cl)nc32)c1]

To a solution of 2-chloro-7H-pyrrolo[2,3-d]pyrimidine (500 mg, 3.267 mmol) in DMF (15 mL) was added potassium carbonate (2.25 g, 16.335 mmol) and the solution stirred for 30 min at rt. A solution of N-(2-(chloromethyl)-4-methylphenyl)-N-methylmethanesulfonamide (968 mg, 3.921 mmol) in 5 mL of DMF was then added to the mixture and it was stirred for 2 h at 100° C. Progress of the reaction was followed by TLC (40% ethyl acetate/hexane). After completion of the reaction, the mixture was diluted with water (50 mL) and extracted with ethyl acetate (75 mL×3). The combined organic layers were washed with water (50 mL×2), followed by brine (50 mL), dried over Na₂SO₄, filtered and evaporated to provide crude product. The resulting residue was purified by Biotage® Isolera™ using 40% ethyl acetate/hexane to give N-(2-((2-chloro-7H-pyrrolo[2,3-d]pyrimidin-7-yl)methyl)-4-methylphenyl)-N-methylmethanesulfonamide (Yield: 1.15 g, 97%). ¹H NMR (400 MHz, CDCl₃): δ 8.79 (s, 1H), 7.33-7.32 (d, 1H, J=4 Hz), 7.19-7.12 (m, 2H), 6.89 (s, 1H), 6.57-6.56 (d, 1H, J=4 Hz), 5.82-5.79 (d, 1H, J=12 Hz), 5.39-5.36 (d, 1H, J=20 Hz), 3.22 (s, 3H), 3.00 (s, 3H), 2.25 (s, 3H).

### Step 8: tert-butyl 4-(4-((7-(5-methyl-2-(N-methylmethylsulfonamido)benzyl)-7H-pyrrolo[2,3-d]pyrimidin-2-yl)amino)phenyl)piperazine-1-carboxylate

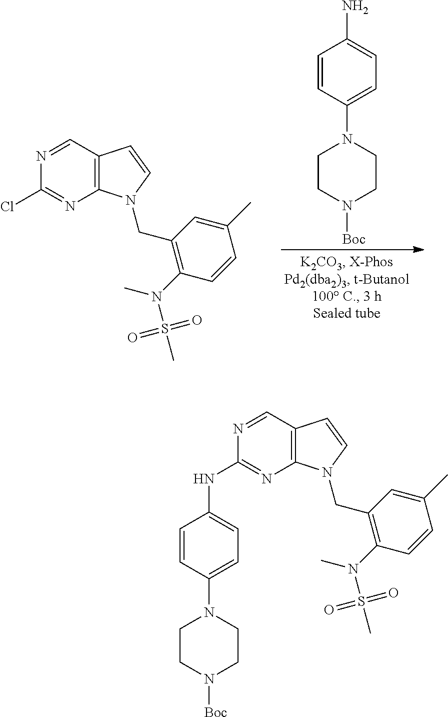 [SMILES: Cc1ccc(N(C)S(C)(=O)=O)c(Cn2ccc3cnc(Cl)nc32)c1 ; CC(C)(C)OC(=O)N1CCN(c2ccc(N)cc2)CC1 ; Cc1ccc(N(C)S(C)(=O)=O)c(Cn2ccc3cnc(Nc4ccc(N5CCN(C(=O)OC(C)(C)C)CC5)cc4)nc32)c1]

N-(2-((2-chloro-7H-pyrrolo[2,3-d]pyrimidin-7-yl)methyl)-4-methylphen yl)-N-methylmethanesulfonamide (250 mg, 0.686 mmol), tert-butyl 4-(4-aminophenyl)piperazine-1-carboxylate (133 mg, 0.480 mmol), potassium carbonate (284 mg, 2.058 mmol) and X-Phos (33 mg, 0.0686 mmol) in tert-butanol (10 mL) were combined under argon atmosphere. The mixture was purged with argon for 30 min and then tris(dibenzylideneacetone)dipalladium (31 mg, 0.0343 mmol) was then added to the mixture. The mixture was stirred for 3 h at 100° C. Progress of the reaction was followed by TLC (5% MeOH/DCM). After completion of the reaction, the mixture was diluted with water (50 mL) and extracted with ethyl acetate (75 mL×2). The combined organic slayer were washed with brine solution (50 mL), dried over Na₂SO₄, filtered and evaporated to provide crude product. The resulting residue was purified by Biotage® Isolera™ system by using 2% MeOH/DCM to give tert-butyl 4-(4-((7-(5-methyl-2-(N-methylmethylsulfonamido)benzyl)-7H-pyrrolo[2,3-d]pyrimidin-2-yl)amino)phenyl)piperazine-1-carboxylate (Yield: 230 mg, 55.4%). ¹H NMR (400 MHz, DMSO-d₆): δ 9.09 (s, 1H), 8.64 (s, 1H), 7.63-7.61 (d, 2H, J=8 Hz), 7.46-7.43 (d, 1H, J=12 Hz), 7.16-7.15 (d, 2H, J=4 Hz), 6.84-6.82 (d, 2H, J=8 Hz), 6.73 (s, 1H), 6.45-6.44 (d, 1H, J=4 Hz), 5.3 (brs, 1H), 5.5 (brs, 1H), 3.45-3.42 (t, 4H, J=8 Hz), 3.11 (s, 3H), 3.08 (s, 3H), 2.97-2.94 (t, 4H, J=8 Hz), 2.14 (s, 3H), 1.40 (s, 9H).

### Step 9: N-methyl-N-(4-methyl-2-((2-((4-(piperazin-1-yl)phenyl)amino)-7H-pyrrolo[2,3-d]pyrimidin-7-yl)methyl)phenyl)methanesulfonamide

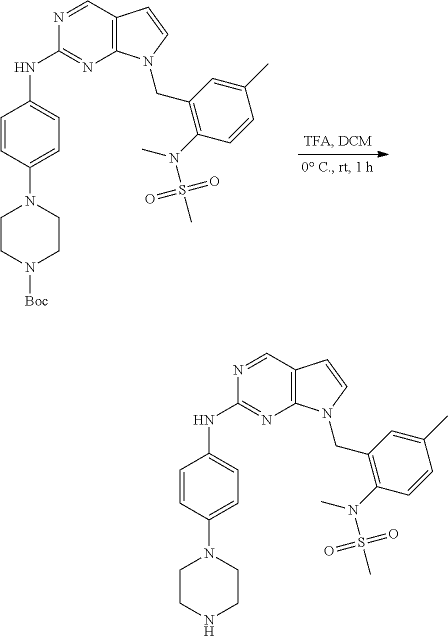 [SMILES: Cc1ccc(N(C)S(C)(=O)=O)c(Cn2ccc3cnc(Nc4ccc(N5CCN(C(=O)OC(C)(C)C)CC5)cc4)nc32)c1 ; Cc1ccc(N(C)S(C)(=O)=O)c(Cn2ccc3cnc(Nc4ccc(N5CCNCC5)cc4)nc32)c1]

A solution of tert-butyl 4-(4-((7-(5-methyl-2-(N-methylmethylsulfonamido)benzyl)-7H-pyrrolo[2,3-d]pyrimidin-2-yl)amino)phenyl)piperazine-1-carboxylate (230 mg, 0.380 mmol) in dichloromethane (20 mL) under argon atmosphere was cooled to 0° C. Trifluoroacetic acid (147 μL, 1.90 mmol) was then added to the mixture and stirred for 1 h at rt. Progress of the reaction was followed by TLC (10% MeOH/DCM). After completion of the reaction, the solvent was evaporated under reduced pressure to give crude residue, which was dissolved in water (10 mL), basified with saturated NaHCO₃ solution and extracted with DCM (50 mL×2). The combined DCM layers were dried over Na₂SO₄, filtered and evaporated to give N-methyl-N-(4-methyl-2-((2-((4-(piperazin-1-yl)phenyl)amino)-7H-pyrrolo[2,3-d]pyrimidin-7-yl)methyl)phenyl) methanesulfonamide (Yield: 170 mg, 89%). ¹H NMR (400 MHz, DMSO-d₆): δ 9.04 (s, 1H), 8.63 (s, 1H), 7.60-7.58 (d, 2H, J=8 Hz), 7.45-7.43 (d, 1H, J=8 Hz), 7.16-7.14 (t, 3H, J=4 Hz), 6.80-6.78 (d, 3H, J=8 Hz), 6.74 (s, 1H), 6.45-6.44 (d, 1H, J=4 Hz) 5.47 (brs, 1H), 5.32 (brs, 1H), 3.10 (s, 3H), 3.080 (s, 3H), 2.92-2.90 (t, 4H, J=4 Hz), 2.82-2.80 (t, 4H, J=4 Hz), 2.14 (s, 3H).

### Example 737

### N-(3-((2-((4-(3-(fluoromethyl)piperazin-1-yl)phenyl)amino)-7H-pyrrolo[2,3-d]pyrimidin-7-yl)methyl)pyridin-2-yl)-N-methylmethanesulfonamide

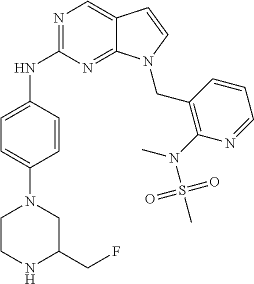 [SMILES: CN(c1ncccc1Cn1ccc2cnc(Nc3ccc(N4CCNC(CF)C4)cc3)nc21)S(C)(=O)=O]

### Step 1: 1-(tert-butyl) 2-methyl 4-(4-nitrophenyl)piperazine-1,2-dicarboxylate

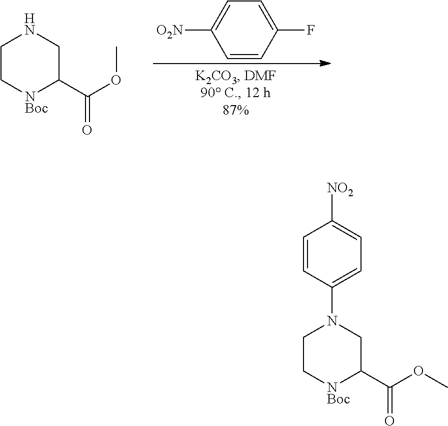 [SMILES: COC(=O)C1CNCCN1C(=O)OC(C)(C)C ; O=[N+]([O-])c1ccc(F)cc1 ; COC(=O)C1CN(c2ccc([N+](=O)[O-])cc2)CCN1C(=O)OC(C)(C)C]

To a stirred solution of 1-tert-butyl 2-methyl piperazine-1,2-dicarboxylate (1.0 g) and K₂CO₃ (1.12 g, 2 eq) in DMF (15 mL), 1-fluoro-4-nitrobenzene (0.86 g, 1.5 eq) was added and the mixture stirred at 90° C. for 12 h. After TLC showed completion of starting material, the mixture was poured into ice cold water and extracted with ethyl acetate (3×100 mL). The organic layer was washed with water (3×50 mL), dried over Na₂SO₄ and concentrated to afford the final compound (1.3 g, 87%) as yellow solid. ¹H NMR (400 MHz, CDCl₃): δ 8.22 (d, 2H), 6.98 (d, 2H), 4.38 (t, 2H), 3.67 (t, 2H). LCMS calculated for (M) 365.39 and found (M+H) 366.21, LCMS showed 86.8% purity.

### Step 2: tert-butyl 2-(hydroxymethyl)-4-(4-nitrophenyl)piperazine-1-carboxylate

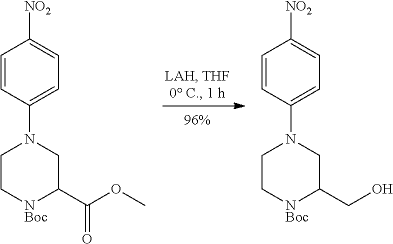 [SMILES: COC(=O)C1CN(c2ccc([N+](=O)[O-])cc2)CCN1C(=O)OC(C)(C)C ; CC(C)(C)OC(=O)N1CCN(c2ccc([N+](=O)[O-])cc2)CC1CO]

To a stirred solution of 1-tert-butyl 2-methyl 4-(4-nitrophenyl)piperazine-1,2-dicarboxylate (1.1 g, 1 eq) in dry THF (15 mL) in a round bottomed blast at 0° C., LAH solution in THF (1 eq) was added drop wise under nitrogen atmosphere, and the mixture stirred at 0° C. for 1 h. After TLC showed completion of starting material, the mixture was quenched with saturated solution of NH₄Cl in ice cold water. The aqueous layer was extracted with ethyl acetate (3×100 mL) and the organic layer was washed with water (3×50 mL), dried over Na₂SO₄ and concentrated to afford the final compound (0.9 g, 96%) as solid. ¹H NMR (400 MHz, CDCl₃): δ 8.13 (d, 2H), 6.79 (d, 2H), 4.26 (brs, 1H), 3.92-3.98 (m, 2H), 3.65-3.78 (m, 3H), 3.30-3.48 (m, 2H), 3.14-3.21 (m, 2H), 1.49 (s, 9H). LCMS calculated for (M) 337.38 and found (M+H) 338.33, LCMS showed 74.6% purity.

### Step 3: (4-(4-nitrophenyl)piperazin-2-yl)methanol

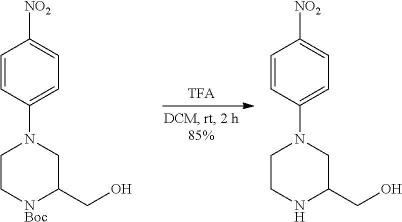 [SMILES: CC(C)(C)OC(=O)N1CCN(c2ccc([N+](=O)[O-])cc2)CC1CO ; O=[N+]([O-])c1ccc(N2CCNC(CO)C2)cc1]

To a solution of tert-butyl 2-(hydroxymethyl)-4-(4-nitrophenyl)piperazine-1-carboxylate (1.0 g, 1.0 eq) in DCM (50 mL) at 0° C., trifluoroacetic acid (5 mL) was added drop wise and the mixture stirred at rt for 2 h. After TLC showed completion of starting material, the mixture was diluted with water (25 mL) and the organic layer separate. The aqueous layer was basified with saturated sodium bicarbonate solution and extracted with EtOAc (2×100 mL). The organic layer was washed with water (3×50 mL), dried over Na₂SO₄ and concentrated to afford (4-(4-nitrophenyl)piperazin-2-yl)methanol (0.65 g, 85%) as off-white solid. LCMS calculated for (M) 237.11 and found (M+H) 238.06, LCMS showed 93.7% purity.

### Step 4: (1-benzyl-4-(4-nitrophenyl)piperazin-2-yl)methanol

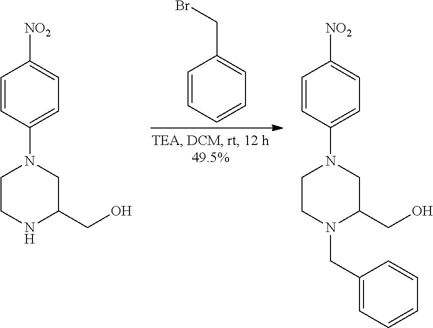 [SMILES: O=[N+]([O-])c1ccc(N2CCNC(CO)C2)cc1 ; BrCc1ccccc1 ; O=[N+]([O-])c1ccc(N2CCN(Cc3ccccc3)C(CO)C2)cc1]

To a stirred solution of (4-(4-nitrophenyl)piperazin-2-yl)methanol (0.6 g, 0.00253 mol) and TEA (0.383 g, 1.5 eq) in DCM (50 mL), benzyl bromide (0.519 g, 1.2 eq) was added and the mixture stirred at rt for 12 h. After TLC showed completion of starting material, the mixture was poured into ice cold water and extracted with DCM (3×50 mL). The organic layer was washed with brine (50 mL), dried over Na₂SO₄ and concentrated. The resulting residue was purified by Combiflash® chromatography on silica gel (230-400#) using methanol in DCM as eluting solvent. The desired product was eluted at 2% methanol in DCM and concentration of the pure fractions provided (1-benzyl-4-(4-nitrophenyl)piperazin-2-yl)methanol (410 mg, 49.5%) as off-white solid. ¹H NMR (400 MHz, CDCl₃): 8.13 (d, 2H), 7.26-7.37 (m, 5H), 6.80 (d, 2H), 4.10 (d, 1H), 3.95 (dd, 1H) 3.66-3.74 (m, 3H), 3.34-3.42 (m, 2H), 3.14-3.15 (m, 1H), 2.95-2.98 (m, 1H), 2.73-2.75 (m, 1H), 2.47-2.50 (m, 1H), 2.25 (brs, 1H). LCMS calculated for (M) 327.38 and found (M+H) 328.0 LCMS showed 89% purity.

### Step 5: 1-benzyl-2-(fluoromethyl)-4-(4-nitrophenyl)piperazine

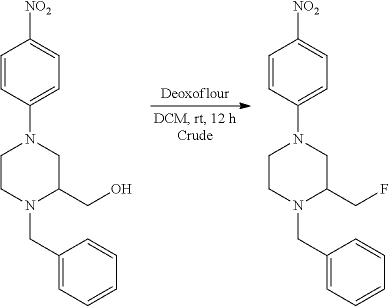 [SMILES: O=[N+]([O-])c1ccc(N2CCN(Cc3ccccc3)C(CO)C2)cc1 ; O=[N+]([O-])c1ccc(N2CCN(Cc3ccccc3)C(CF)C2)cc1]

To a stirred solution of (1-benzyl-4-(4-nitrophenyl)piperazin-2-yl)methanol (0.350 g, 0.00107 mol) in DCM (50 mL), bis(2-methoxyethyl)aminosulfur trifluoride (Deoxo-Fluor) (0.47 g, 2.0 eq) was added and the mixture stirred at rt for 12 h. After TLC showed completion of starting material, the mixture was poured into ice cold water and extracted with DCM (3×50 mL). The organic layer was washed with brine (50 mL), dried over Na₂SO₄ and concentrated to afford 1-benzyl-2-(fluoromethyl)-4-(4-nitrophenyl)piperazine (0.415 g, Crude) as solid, and used in the next step without any purification. LCMS calculated for (M) 329.15 and found (M+H) 330.22, LCMS showed 64.99% purity.

### Step 6: 4-(4-benzyl-3-(fluoromethyl)piperazin-1-yl)aniline

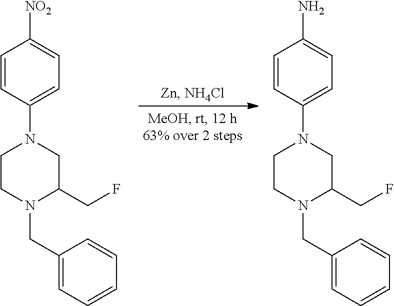 [SMILES: O=[N+]([O-])c1ccc(N2CCN(Cc3ccccc3)C(CF)C2)cc1 ; Nc1ccc(N2CCN(Cc3ccccc3)C(CF)C2)cc1]

To a solution of 1-benzyl-2-(fluoromethyl)-4-(4-nitrophenyl)piperazine (0.410 g, 1.0 eq) in methanol (50 mL), Zn (0.488 g, 6 eq) and ammonium chloride (0.399 g, 6 eq) were added and stirred at rt for 12 h. After TLC showed completion of starting material, the mixture was filtered through a Celite® bed and washed with methanol. The filtrate was concentrated and diluted with water (25 mL). The aqueous layer was extracted with ethyl acetate (3×100 mL) and the organic layer was washed with water (20 mL), dried over Na₂SO₄ and concentrated to afford 4-(4-benzyl-3-(fluoromethyl)piperazin-1-yl)aniline (0.202 g, 63% over 2 steps) as off-white solid. LCMS calculated for (M) 299.18 and found (M+H) 300.12, LCMS showed 68.4% purity.

### Step 7: N-(3-((2-((4-(4-benzyl-3-(fluoromethyl)piperazin-1-yl)phenyl)amino)-7H-pyrrolo[2,3-d]pyrimidin-7-yl)methyl)pyridin-2-yl)-N-methylmethanesulfonamide

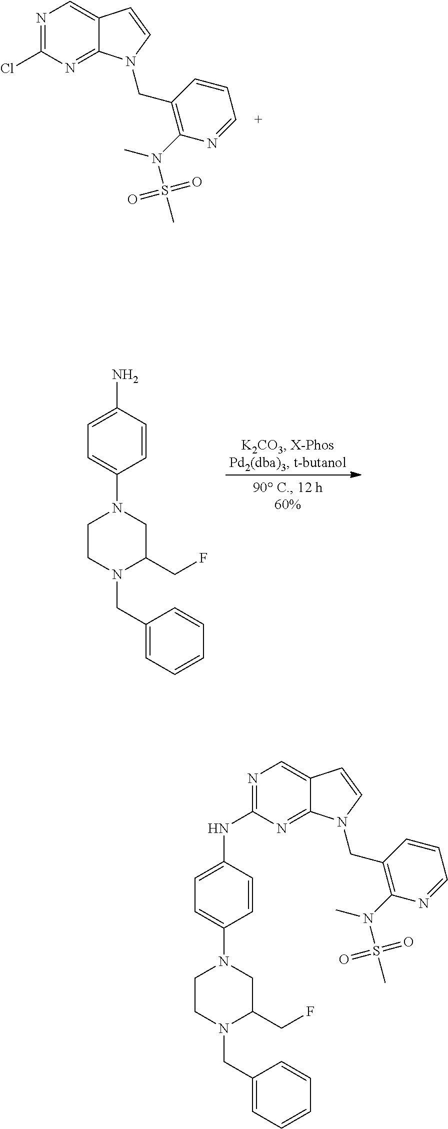

A stirred mixture of 4-(4-benzyl-3-(fluoromethyl)piperazin-1-yl)aniline (0.15 g, 1.0 eq), N-(2-((2-chloro-7H-pyrrolo[2,3-d]pyrimidin-7-yl)methyl)phenyl)-N-methylmethanesulfonamide (0.211 g, 1.2 eq), K₂CO₃ (0.207 g, 3.0 eq), and t-butanol (15 mL) in a vial was degassed with an nitrogen balloon for 15 min at rt, and then X-Phos (0.1 eq), Pd₂(dba)₃ (0.05 eq) were added and again degassed for 5 min. After closing the vial, the mixture was heated at 90° C. for 12 h. After completion of starting material, the mixture was cooled to rt, diluted with EtOAc (50 mL) and filtered over a Celite® bed. The filtrate was concentrated and resulting crude was purified by column chromatography on silica gel (100-200#) using methanol in DCM as eluent. The product eluted at 2.5% methanol in DCM and the concentration of pure fractions provided N-(3-((2-((4-(4-benzyl-3-(fluoromethyl)piperazin-1-yl)phenyl)amino)-7H-pyrrolo[2,3-d]pyrimidin-7-yl)methyl)pyridin-2-yl)-N-methylmethanesulfonamide (0.18 g, 60%) as off-white solid. LCMS calculated for (M) 614.26 and found (M+H) 615.49, LCMS showed 95.88% purity.

### Step 8: N-(3-((2-((4-(3-(fluoromethyl)piperazin-1-yl)phenyl)amino)-7H-pyrrolo[2,3-d]pyrimidin-7-yl)methyl)pyridin-2-yl)-N-methylmethanesulfonamide

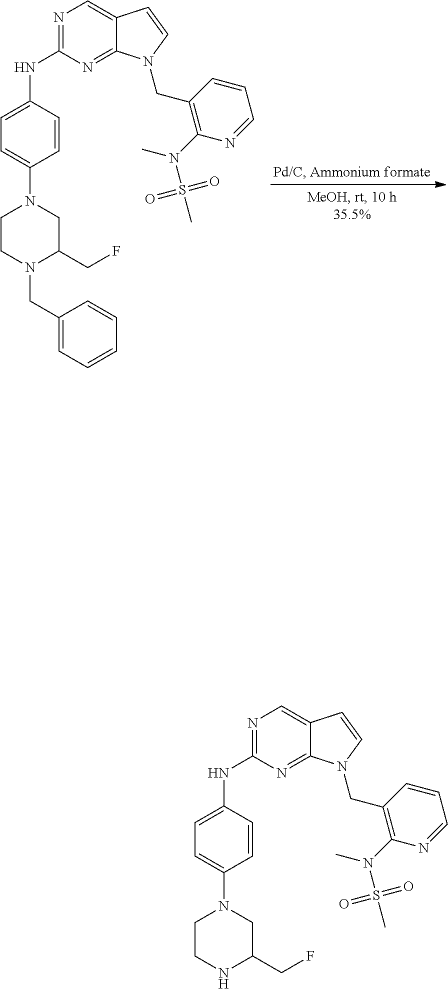 [SMILES: CN(c1ncccc1Cn1ccc2cnc(Nc3ccc(N4CCN(Cc5ccccc5)C(CF)C4)cc3)nc21)S(C)(=O)=O ; CN(c1ncccc1Cn1ccc2cnc(Nc3ccc(N4CCNC(CF)C4)cc3)nc21)S(C)(=O)=O]

To a stirred solution of N-(3-((2-((4-(4-benzyl-3-(fluoromethyl)piperazin-1-yl)phenyl)amino)-7H-pyrrolo[2,3-d]pyrimidin-7-yl)methyl)pyridin-2-yl)-N-methylmethanesulfonamide (0.07 g, 1.0 eq) in methanol (20 mL) under nitrogen atmosphere, 10% Pd/C (0.02 g, 50% wet) and ammonium formate (0.036 g, 5.0 eq) were added. The resulting mixture was stirred at rt for 10 h. After completion of the starting material, the mixture was filtered through a Celite® bed and the filtrate was concentrated. The resulting residue was purified by column chromatography on silica gel (100-200#) using methanol in DCM as the eluent. The product eluted at 5% methanol in DCM and concentration of pure fractions afforded N-(3-((2-((4-(3-(fluoromethyl)piperazin-1-yl)phenyl)amino)-7H-pyrrolo[2,3-d]pyrimidin-7-yl)methyl)pyridin-2-yl)-N-methylmethanesulfonamide as off-white solid (0.021 g, 35.5%). 1H NMR (400 MHz, DMSO-d6): δ 9.134 (s, 1H), 8.672 (s, 1H), 7.468 (brs, 1H), 7.553 (d, 2H), 7.368 (d, 1H), 7.293 (d, 1H), 7.242 (d, 1H), 6.814 (d, 2H), 6.489 (d, 1H), 5.481 (s, 2H), 4.503 (brs, 1H), 4.356 (brs, 1H), 3.431 (m, 1H), 3.281 (m, 1H), 3.168 (s, 6H), 3.070-2.991 (m, 3H), 2.852 (m, 1H), 2.391 (m, 1H). LCMS calculated for (M) 524.21 and found (M+H) 525.17, LCMS showed 98.48% purity.

### Example 738

### 2-((1-(4-((5,5-Dimethyl-7-(quinolin-8-ylsulfonyl)-6,7-dihydro-5H-pyrrolo[2,3-d]pyrimidin-2-yl)amino)phenyl)piperidin-4-yl)(methyl)amino)ethan-1-ol

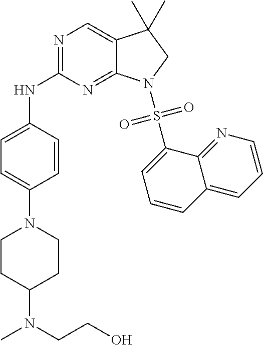 [SMILES: CN(CCO)C1CCN(c2ccc(Nc3ncc4c(n3)N(S(=O)(=O)c3cccc5cccnc35)CC4(C)C)cc2)CC1]

### Step 1: tert-butyl (1-(4-nitrophenyl)piperidin-4-yl)carbamate

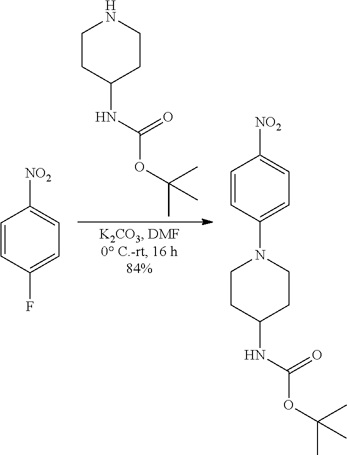

To a stirred solution of tert-butyl piperidin-4-ylcarbamate (7.0 g, 1.0 eq) in DMF (100 mL), 4-fluoronitrobenzene (11.0 g, 1.1 eq) and K₂CO₃ (21.0 g, 3.0 eq) were added and stirred at rt for 16 h. After TLC showed completion of starting material, the mixture was diluted with water (120 mL) and filtered. The solid was washed with excess water and dried to get tert-butyl (1-(4-nitrophenyl)piperidin-4-yl)carbamate (13.5 g, 84%) as yellow solid. 1H NMR (400 MHz, DMSO-d6): δ 8.012 (d, 2H), 7.982 (d, 2H), 6.868 (s, 1H), 3.963 (d, 2H), 3.53 (d, 2H), 3.41 (s, 1H), 3.06 (t, 2H), 1.85 (d, 2H), 1.33 (s, 9H).

### Step 2: tert-butyl methyl(1-(4-nitrophenyl)piperidin-4-yl)carbamate

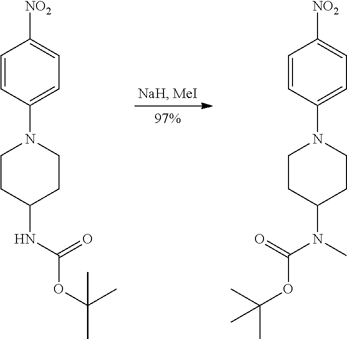 [SMILES: CC(C)(C)OC(=O)NC1CCN(c2ccc([N+](=O)[O-])cc2)CC1 ; CN(C(=O)OC(C)(C)C)C1CCN(c2ccc([N+](=O)[O-])cc2)CC1]

To a stirred solution of tert-butyl (1-(4-nitrophenyl)piperidin-4-yl)carbamate (3.0 g, 1.0 eq) in DMF (20 mL) at 0° C., NaH (2.5 eq, 60%) was added portion wise and stirred at 0° C. for 15 min. To this mixture, methyl iodide (3.0 eq) was added and stirred at rt for 2 h. After TLC showed completion of starting material, the mixture was poured into saturated NH₄Cl solution (60 mL) and extracted with EtOAc (2×80 mL). The organic layer was dried over anhydrous Na₂SO₄, filtered and concentrated to obtain tert-butyl methyl(1-(4-nitrophenyl)piperidin-4-yl)carbamate (3.0 g, 97%) as pale yellow solid. 1H NMR (400 MHz, CDCl₃): δ 8.101 (d, 2H), 6.829 (d, 2H), 4.11 (d, 2H), 3.02 (t, 2H), 2.95 (s, 1H), 2.71 (s, 3H), 1.62 (d, 4H), 1.41 (s, 9H).

### Step 3: N-methyl-1-(4-nitrophenyl)piperidin-4-amine hydrochloride

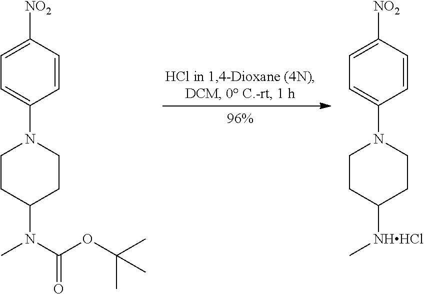

To a solution of tert-butyl methyl(1-(4-nitrophenyl)piperidin-4-yl)carbamate (2.0 g, 1.0 eq) in DCM (50 mL) at rt, 4N HCl in 1,4-dioxane (10 mL) was added and stirred at rt for 1 h. After TLC showed completion of starting material, solvent was evaporated and resulting residue was co-distilled with diethyl ether (10 ml) and dried to provide N-methyl-1-(4-nitrophenyl)piperidin-4-amine hydrochloride (2.1 g, 96%) as pale yellow solid. LCMS calculated for (M−HCl) 235.10 and found (M+H−HCl) 236.20, LCMS showed 99.65% purity.

### Step 4: (2-bromoethoxy) (tert-butyl)dimethylsilane

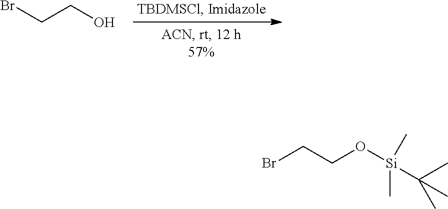

To a solution of 2-bromoethan-1-ol (6.5 g, 1.0 eq) in ACN (100 mL) at rt, TBDMSCl (5.0 g, 0.65 eq) and imidazole (3.4 g, 1.0 eq) were added and stirred at rt for 12 h. After TLC showed completion of starting material, the mixture was diluted with water (90 mL) and extracted with EtOAc (2×80 mL). The organic layer was washed with brine solution (20 mL), dried over anhydrous Na₂SO₄ and concentrated to provide (2-bromoethoxy)(tert-butyl)dimethylsilane (7.5 g, 57%). 1H NMR (400 MHz, CDCl₃): δ 3.87 (t, 2H), 3.47 (t, 2H), 0.86 (s, 9H), 0.06 (s, 6H).

### Step 5: N-(2-((tert-butyldimethylsilyl)oxy)ethyl)-N-methyl-1-(4-nitrophenyl)piperidin-4-amine

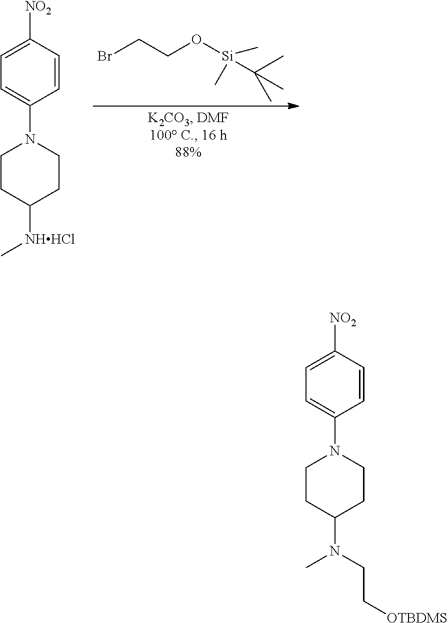

To stirred mixture of N-methyl-1-(4-nitrophenyl)piperidin-4-amine hydrochloride (1.8 g, 1.0 eq) in DMF (10 mL), (2-bromoethoxy)(tert-butyl)dimethylsilane (3.1 g, 2.0 eq) and K₂CO₃ (5.4 g, 6.0 eq) were added and heated to 100° C. for 16 h. After TLC showed completion of starting material, the mixture was diluted with water (70 mL) and extracted with EtOAc (2×50 mL). The organic layer was washed with brine solution (20 mL), dried over anhydrous Na₂SO₄ and concentrated to provide N-(2-((tert-butyldimethylsilyl)oxy)ethyl)-N-methyl-1-(4-nitrophenyl)piperidin-4-amine (2.25 g, 88%) as pale brown gum. 1H NMR (400 MHz, CDCl₃): δ 8.101 (d, 2H), 6.802 (d, 2H), 4.14 (dd, 1H), 4.003 (d, 1H) 3.70 (d, 2H), 3.12 (s, 3H), 3.02 (d, 2H), 2.81 (s, 1H), 2.56 (t, 2H), 2.43 (s, 2H), 1.39 (t, 2H), 1.25 (s, 9H), 0.07 (s, 6H).

### Step 6: 1-(4-aminophenyl)-N-(2-((tert-butyldimethylsilyl)oxy)ethyl)-N-methylpiperidin-4-amine

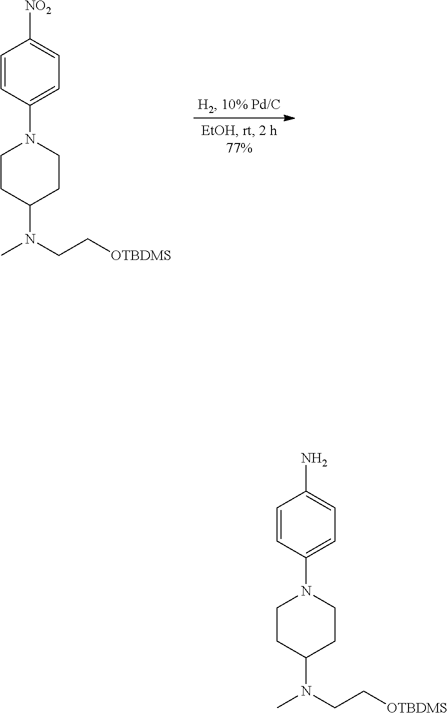 [SMILES: CN(CCO[Si](C)(C)C(C)(C)C)C1CCN(c2ccc([N+](=O)[O-])cc2)CC1 ; CN(CCO[Si](C)(C)C(C)(C)C)C1CCN(c2ccc(N)cc2)CC1]

To a solution of N-(2-((tert-butyldimethylsilyl)oxy)ethyl)-N-methyl-1-(4-nitrophenyl)piperidin-4-amine (0.8 g, 1.0 eq) in ethanol (20 mL) at rt, 10% Pd/C (0.5 g, 50% wet) was added and the mixture stirred under hydrogen atmosphere for 2 h. After TLC showed completion of starting material, the mixture was filtered through a Celite® bed and washed with methanol (20 mL). The filtrate was concentrated to afford 1-(4-aminophenyl)-N-(2-((tert-butyldimethylsilyl)oxy)ethyl)-N-methylpiperidin-4-amine (0.30 g, 81%) as pale brown gum. LCMS calculated for (M) 363.37 and found (M+H) 364.47 LCMS showed 66.52% purity.

### Step 7: N-(4-(4-((2-((tert-butyldimethylsilyl)oxy)ethyl)(methyl)amino)piperidin-1-yl)phenyl)-5,5-dimethyl-7-(quinolin-8-ylsulfonyl)-6,7-dihydro-5H-pyrrolo[2,3-d]pyrimidin-2-amine

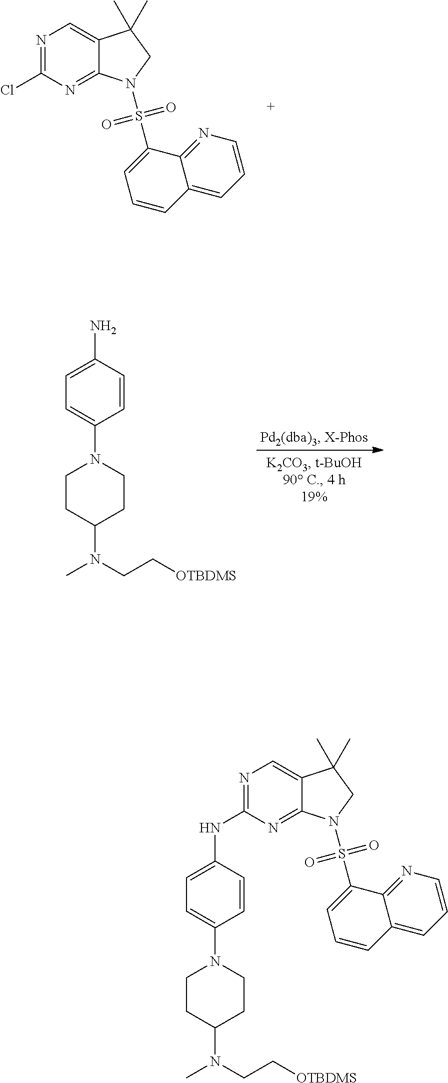 [SMILES: CC1(C)CN(S(=O)(=O)c2cccc3cccnc23)c2nc(Cl)ncc21 ; "+" ; CN(CCO[Si](C)(C)C(C)(C)C)C1CCN(c2ccc(N)cc2)CC1 ; CN(CCO[Si](C)(C)C(C)(C)C)C1CCN(c2ccc(Nc3ncc4c(n3)N(S(=O)(=O)c3cccc5cccnc35)CC4(C)C)cc2)CC1]

A stirred mixture of 8-((2-chloro-5,5-dimethyl-5,6-dihydro-7H-pyrrolo[2,3-d]pyrimidin-7-yl)sulfonyl)quinoline (0.40 g, 1.0 eq), 1-(4-aminophenyl)-N-(2-((tert-butyldimethylsilyl)oxy)ethyl)-N-methylpiperidin-4-amine (0.348 g, 0.9 eq) and K₂CO₃ (0.441 g, 3.0 eq) in t-butanol (15.0 mL) in a vial was degassed using an argon balloon for 15 min. X-Phos (0.135 g, 0.1 eq) and Pd₂(dba)₃ (0.130 g, 0.05 eq) were added to this mixture and degassing continued for another 10 min. The vial was closed and the contents were heated at 90° C. for 4 h. After completion of the starting material, the mixture was quenched with water and extracted with EtOAc (2×50 mL). The organic layer was dried over anhydrous Na₂SO₄, filtered and concentrated. The resulting residue was purified by Combiflash® chromatography on neutral alumina column using EtOAc in hexane as the eluent. The desired product eluted at 50% EtOAc in hexane. The fractions with product were concentrated to obtain N-(4-(4-((2-((tert-butyldimethylsilyl)oxy)ethyl)(methyl)amino)piperidin-1-yl)phenyl)-5,5-dimethyl-7-(quinolin-8-ylsulfonyl)-6,7-dihydro-5H-pyrrolo[2,3-d]pyrimidin-2-amine as pale brown solid (0.14 g, 19%). LCMS calculated for (M) 701.35 and found (M+H) 702.29. LCMS showed 85.49% purity.

### Step 8: 2-((1-(4-((5,5-Dimethyl-7-(quinolin-8-ylsulfonyl)-6,7-dihydro-5H-pyrrolo[2,3-d]pyrimidin-2-yl)amino)phenyl)piperidin-4-yl)(methyl)amino)ethan-1-ol

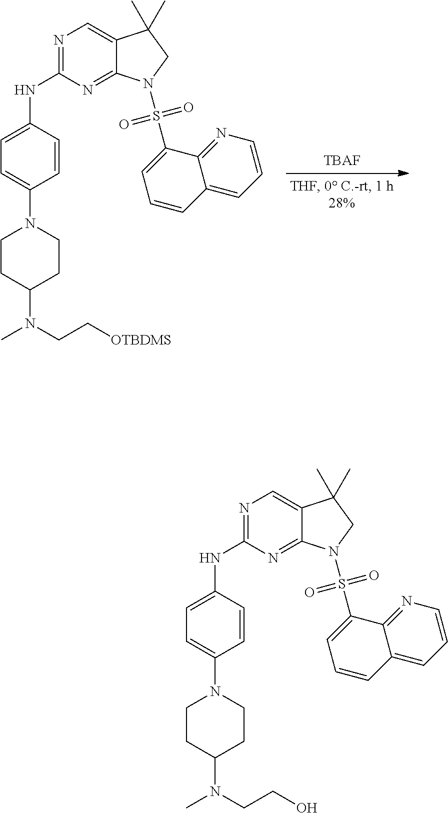 [SMILES: CN(CCO[Si](C)(C)C(C)(C)C)C1CCN(c2ccc(Nc3ncc4c(n3)N(S(=O)(=O)c3cccc5cccnc35)CC4(C)C)cc2)CC1 ; "TBAF" ; CN(CCO)C1CCN(c2ccc(Nc3ncc4c(n3)N(S(=O)(=O)c3cccc5cccnc35)CC4(C)C)cc2)CC1]

To a stirred solution of N-(4-(4-((2-((tert-butyldimethylsilyl)oxy)ethyl)(methyl)amino)piperidin-1-yl)phenyl)-5,5-dimethyl-7-(quinolin-8-ylsulfonyl)-6,7-dihydro-5H-pyrrolo[2,3-d]pyrimidin-2-amine (0.13 g, 1.0 eq) in THF (10 mL) at 0° C., tetrabutyl ammonium fluoride (1.4 mL, 1.0M solution in THF, 3.0 eq) was added and the mixture was stirred at rt for 1 h. After completion of the starting material, the reaction was quenched with saturated sodium bicarbonate solution and extracted with EtOAc (2×50 mL). The organic layer was dried over Na₂SO₄, filtered and concentrated to obtain crude residue. The crude was purified by Combiflash® chromatography on silica gel (230-400#) using methanol in DCM as eluent. The product eluted at 7% methanol in DCM and concentration of fractions provided 2-((1-(4-((5,5-dimethyl-7-(quinolin-8-ylsulfonyl)-6,7-dihydro-5H-pyrrolo[2,3-d]pyrimidin-2-yl)amino)phenyl)piperidin-4-yl)(methyl)amino)ethan-1-ol as pale brown solid (0.03 g, 28%). 1H NMR (400 MHz, DMSO-d6): δ 9.038 (dd, 2H), 8.723 (d, 1H), 8.501 (d, 1H), 8.284 (d, 1H), 7.986 (s, 1H), 7.744-7.657 (m, 2H), 7.328 (d, 2H), 6.880 (d, 2H), 4.440 (s, 2H), 3.706 (d, 2H), 3.499 (brs, 2H), 2.621 (t, 3H), 2.591 (m, 1H), 2.315 (brs, 2H), 1.817 (d, 2H), 1.589 (d, 2H), 1.359 (s, 6H), 1.228 (d, 2H). LCMS calculated for (M) 587.27 and found (M+H) 588.22, LCMS showed 97.21% purity.

### Synthesis of 2-chloro-5,5-dimethyl-6,7-dihydro-5H-pyrrolo[2,3-d]pyrimidine

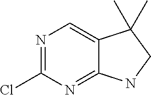

### Step 1: 5-bromo-2-chloropyrimidin-4-amine

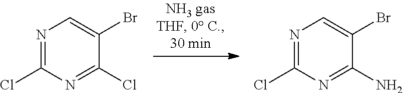 [SMILES: Clc1ncc(Br)c(Cl)n1 ; Nc1nc(Cl)ncc1Br]

Ammonia gas was purged into a solution of 5-bromo-2,4-dichloropyrimidine (25.0 g, 1.0 eq) in THF (250 mL) under nitrogen atmosphere at 0° C. for 1 h. After completion of starting material on TLC, the mixture was quenched with ice cold water and extracted with EtOAc (2×500 mL). The organic layer was dried over anhydrous Na₂SO₄, filtered and concentrated to obtain as off-white solid (20 g, 87%). 1H NMR (400 MHz, CDCl₃): δ 8.237 (s, 1H), 5.650 (brs, 2H).

### Step 2: 5-bromo-2-chloro-N-(2-methylallyl)pyrimidin-4-amine

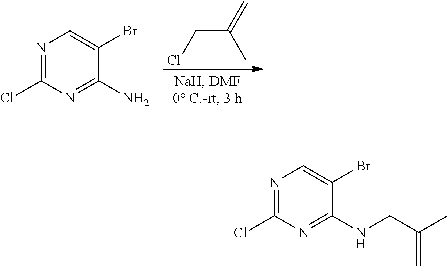

To a solution of 5-bromo-2-chloropyrimidin-4-amine (15.0 g, 1.0 eq) in DMF (150 mL) at 0° C. under nitrogen atmosphere, sodium hydride (2.64 g, 1.5 eq) was added portion wise and the mixture stirred for 10 min. To this mixture, 3-chloro-2-methylprop-1-ene (6.93 g, 1.05 eq) was added and reaction was stirred at rt for 3 h. After completion of starting material on TLC, the mixture was quenched with ice cold water and extracted with EtOAc (2×500 mL). The organic layer was dried over anhydrous Na₂SO₄, filtered and concentrated. The resulting residue was purified by Combiflash® chromatography using EtOAc in hexane as eluent. The desired product was eluted at 10% EtOAc in hexane. The fractions with product were concentrated to obtain 5-bromo-2-chloro-N-(2-methylallyl)pyrimidin-4-amine as light yellow gummy liquid (10.0 g, 52%). 1H NMR (400 MHz, DMSO-d6): δ 8.256 (s, 1H), 7.967 (t, 1H), 4.778 (d, 1H), 4.697 (d, 1H), 3.898 (d, 2H), 1.700 (s, 3H).

### Step 3: 2-chloro-5,5-dimethyl-6,7-dihydro-5H-pyrrolo[2,3-d]pyrimidine

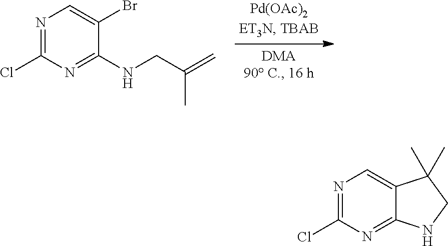

To a solution of 5-bromo-2-chloro-N-(2-methylallyl)pyrimidin-4-amine (4.3 g, 1.0 eq) in dimethylacetamide (DMA; 50 mL) under nitrogen atmosphere (in 5 vials), TBAB (5.83 g, 1.1 eq) was added and the mixture stirred for 5 min. The reaction was degassed for 15 min using argon, Et₃N (4.8 g, 3.0 eq) was added followed by degassing for 10 min. To this mixture, Pd(OAc)₂ (0.37 g, 0.1 eq) was added and the vials sealed. The contents were heated at 90° C. for 16 h. After completion of starting material on TLC, the mixture was poured into water (250 mL) and extracted with EtOAc (2×500 mL). The organic layer was dried over anhydrous Na₂SO₄, filtered and concentrated. The resulting residue was purified by Combiflash® chromatography using EtOAc in hexane as eluent. The desired product was eluted at 35% EtOAc in hexane. The fractions with product were concentrated to obtain 2-chloro-5,5-dimethyl-6,7-dihydro-5H-pyrrolo[2,3-d]pyrimidine as yellow solid (0.5 g, 16%). 1H NMR (400 MHz, DMSO-d6): δ 7.937 (s, 1H), 7.793 (s, 1H), 1.360 (s, 6H), 1.101 (m, 2H).

### 2-Chloro-5,5-dimethyl-7-(naphthalen-1-ylsulfonyl)-6,7-dihydro-5H-pyrrolo[2,3-d]pyrimidine

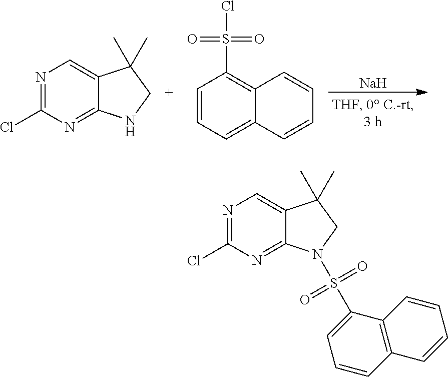

To a solution of 2-chloro-5,5-dimethyl-6,7-dihydro-5H-pyrrolo[2,3-d]pyrimidine (0.5 g, 1.0 eq) in THF (10 mL) under nitrogen atmosphere at 0° C., NaH (0.11 g, 1.7 eq) was added portion wise and stirred for 5 min. To this mixture, naphthalene-1-sulfonyl chloride (0.74 g, 1.2 eq) was added and the reaction stirred at rt for 3 h. After completion of starting material on TLC, the mixture was poured into ice cold water (100 mL) and extracted with EtOAc (2×100 mL). The organic layer was dried over anhydrous Na₂SO₄, filtered and concentrated to obtain 2-chloro-5,5-dimethyl-7-(naphthalen-1-ylsulfonyl)-6,7-dihydro-5H-pyrrolo[2,3-d]pyrimidine as yellow solid (0.75 g, 75%). 1H NMR (400 MHz, DMSO-d6): δ 8.617 (d, 1H), 8.461 (d, 1H), 8.366 (d, 1H), 8.339 (s, 1H), 8.137 (d, 1H), 7.786 (m, 2H), 7.690 (t, 1H), 4.098 (s, 2H), 1.231 (s, 6H).

### Synthesis of N¹,N¹-dimethylbenzene-1,3-diamine

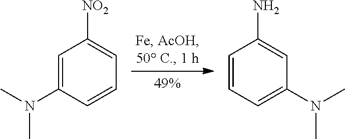

To a stirred mixture of N,N-dimethyl-3-nitroaniline (0.5 g, 1.0 eq) in AcOH (10 mL) at 0° C. in a vial, iron powder (5 eq) was added and the contents heated at 50° C. for 1 h. After completion of starting material, the mixture was quenched with ice water (100 mL) and basified with NaHCO₃. The aqueous layer was extracted with EtOAc (2×100 mL) and the organic layer was dried over anhydrous Na₂SO₄, filtered and concentrated. The resulting residue was purified by column chromatography by using 100-200# silica gel and eluted with 40% EtOAc in hexane to obtain N¹,N¹-dimethylbenzene-1,3-diamine as yellow solid (198 mg, 49%). ¹H NMR (400 MHz, DMSO-d6): δ 6.8 (brs, 1H), 5.935 (d, 3H), 4.777 (s, 2H), 2.793 (s, 6H).

### Synthesis of (S)-1-((R)-4-(4-aminophenyl)-2-methylpiperazin-1-yl)propan-2-ol

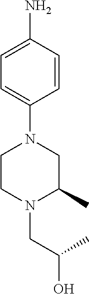 [SMILES: C[C@H](O)CN1CCN(c2ccc(N)cc2)C[C@H]1C]

### Step 1: (R)-3-methyl-1-(4-nitrophenyl)piperazine

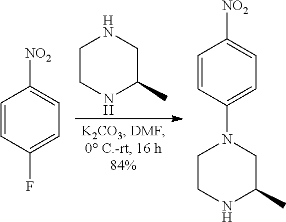

To a stirred mixture of 4-fluoronitrobenzene (2.0 g, 0.014 mol, 1.0 eq) in DMF (50 mL) at rt, (R)-2-methylpiperazine (1.1 eq) and K₂CO₃ (3.0 eq) were added and stirred at rt for 16 h. After TLC showed completion of starting material, the mixture was diluted with water (100 mL) and extracted with EtOAc (3×150 mL). The organic layer was washed with brine solution (50 mL), dried over anhydrous sodium sulphate, and concentrated to provide crude residue. The crude was triturated with n-hexane and filtered to obtain (R)-3-methyl-1-(4-nitrophenyl)piperazine (2.6 g, 84%) as yellow solid. 1H NMR (400 MHz, DMSO-d6): δ 8.028 (d, 2H), 7.002 (d, 2H), 3.860 (m, 2H), 2.952 (d, 1H), 2.887 (t, 1H), 2.790 (m, 2H), 2.450 (m, 1H), 2.331 (s, 1H), 1.023 (d, 3H).

### Step 2: (S)-1-((R)-2-methyl-4-(4-nitrophenyl)piperazin-1-yl)propan-2-ol

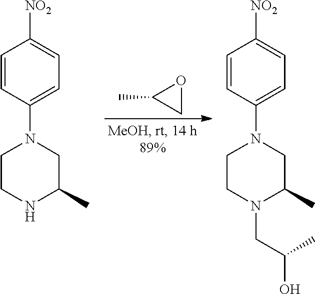

To a stirred solution of (R)-3-methyl-1-(4-nitrophenyl)piperazine (1.5 g, 1.0 eq) in MeOH (50 mL), (S)-2-methyloxirane (0.6 ml, 1.5 eq) was added and stirred at rt for 14 h. After TLC showed completion of starting material, the mixture was concentrated to obtain (S)-1-((R)-2-methyl-4-(4-nitrophenyl)piperazin-1-yl)propan-2-ol (1.77 g, 89%) as pale yellow solid. LCMS calculated for (M) 279.30 and found (M+H) 280.30, LCMS showed 98.27% purity.

### Step 3: (S)-1-((R)-4-(4-aminophenyl)-2-methylpiperazin-1-yl)propan-2-ol

To a solution of (S)-1-((R)-2-methyl-4-(4-nitrophenyl)piperazin-1-yl)propan-2-ol (1.7 g, 1.0 eq) in methanol (200 mL) at rt, 10% Pd/C (3.6 g, 50% wet) was added and stirred under hydrogen atmosphere for 2 h. After TLC showed completion of starting material, the mixture was filtered through a Celite® bed and washed with methanol (25 mL). The filtrate was concentrated to afford (S)-1-((R)-4-(4-aminophenyl)-2-methylpiperazin-1-yl)propan-2-ol (1.2 g, 80%) as pale brown solid. LCMS calculated for (M) 249.10 and found (M+H) 250.20 LCMS showed 92.73% purity.

### Synthesis of tert-butyl (1-(4-aminophenyl)piperidin-3-yl)carbamate

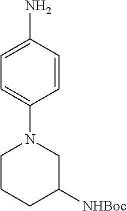 [SMILES: CC(C)(C)OC(=O)NC1CCCN(c2ccc(N)cc2)C1]

### Step 1: tert-butyl (1-(4-nitrophenyl)piperidin-3-yl)carbamate

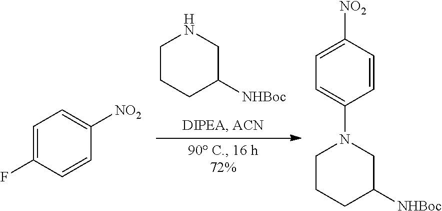 [SMILES: O=[N+]([O-])c1ccc(F)cc1 ; CC(C)(C)OC(=O)NC1CCCNC1 ; CC(C)(C)OC(=O)NC1CCCN(c2ccc([N+](=O)[O-])cc2)C1]

To stirred mixture of 4-fluoronitrobenzene (0.5 g, 0.0035 mol, 1.0 eq) in ACN (10 mL), tert-butyl piperidin-3-ylcarbamate (0.70 g, 0.0035 mol, 1.0 eq) and DIPEA (0.7 mL, 0.0035 mol, 1.0 eq) were added and heated at 90° C. for 16 h. After TLC showed completion of starting material, the mixture was cooled to rt, diluted with water (40 mL) and extracted with EtOAc (2×40 mL). The organic layer was washed with brine solution (20 mL), dried over anhydrous Na₂SO₄ and concentrated to get crude residue. The residue was triturated with n-pentane and filtered to obtain tert-butyl (1-(4-nitrophenyl)piperidin-3-yl)carbamate (0.81 g, 72%) as yellow solid. 1H NMR (400 MHz, DMSO-d6): δ 8.04 (d, 2H), 6.98 (d, 2H), 6.95 (s, 1H), 3.86 (t, 2H), 3.30 (s, 1H), 3.09 (d, 2H), 1.84 (s, 2H), 1.53 (d, 2H), 1.39 (s, 9H).

### Step 2: tert-butyl (1-(4-aminophenyl)piperidin-3-yl)carbamate

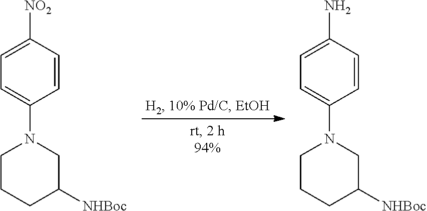 [SMILES: CC(C)(C)OC(=O)NC1CCCN(c2ccc([N+](=O)[O-])cc2)C1 ; CC(C)(C)OC(=O)NC1CCCN(c2ccc(N)cc2)C1]

To a solution of tert-butyl (1-(4-nitrophenyl)piperidin-3-yl)carbamate (0.8 g, 1.0 eq) in ethanol (200 ml) at rt, 10% Pd/C (0.4 g, 50% wet) was added and the mixture was hydrogenated at rt for 2 h. After TLC showed completion of starting material, the mixture was filtered through a Celite® bed and washed with methanol (50 mL). The filtrate was concentrated to afford tert-butyl (1-(4-aminophenyl)piperidin-3-yl)carbamate (0.65 g, 94%) as pale brown gum. LCMS calculated for (M) 291.10 and found (M+H) 292.20 LCMS showed 79.70% purity.

### Synthesis of tert-butyl (1-(4-aminophenyl)piperidin-4-yl)(cyclopropyl)carbamate

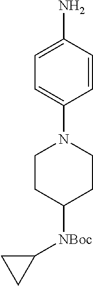 [SMILES: CC(C)(C)OC(=O)N(C1CC1)C1CCN(c2ccc(N)cc2)CC1]

### Step 1: 1-(4-nitrophenyl) piperidin-4-one

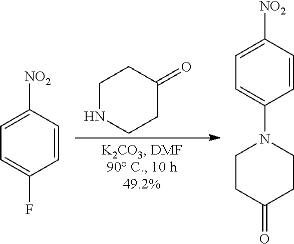

A stirred mixture of 4-fluoronitrobenzene (1.5 g, 1.0 eq), piperidin-4-one (1.5 eq) K₂CO₃ (3 eq), and DMF (12 mL) in a vial was heated at 90° C. for 10 h. After completion of starting material, the mixture was poured in ice water (200 mL) and extracted with EtOAc (2×200 mL). The organic layer was dried over anhydrous Na₂SO₄, filtered and concentrated. The resulting residue was purified by column chromatography by using 100-200# silica gel and eluted with 17% EtOAc in hexane to obtain 1-(4-nitrophenyl) piperidin-4-one (1.35 g, 49.2%) as yellow solid. 1H NMR (400 MHz, DMSO-d6): δ 8.088 (d, 2H), 7.045 (d, 2H), 3.835 (t, 4H), 2.527 (t, 4H).

### Step 2: N-cyclopropyl-1-(4-nitrophenyl) piperidin-4-amine

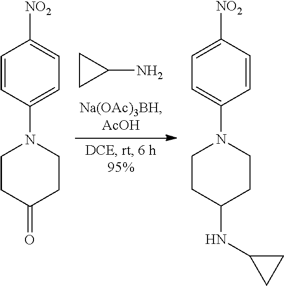

To a stirred solution of 1-(4-nitrophenyl) piperidin-4-one (1.2 g, 1.0 eq) in DCE (15 mL) in a two necked round bottomed flask under nitrogen atmosphere at 0° C., sodium triacetoxyborohydride (2.0 eq) was added. AcOH (1.2 mL) and cyclopropylamine (1.5 eq) were then added portion wise to the mixture. The mixture was allowed to stir at rt for 6 h. After completion of starting material, the mixture was poured into water, neutralised with 10% NaOH solution and extracted with DCM (2×50 mL). The organic layer was dried over Na₂SO₄ and concentrated. The resulting crude was washed with pentane to provide N-cyclopropyl-1-(4-nitrophenyl) piperidin-4-amine (1.3 g, 95%) as yellow orange gummy compound. 1H NMR (400 MHz, DMSO-d6): δ 8.023 (d, 2H), 7.001 (d, 2H), 3.936 (d, 2H), 3.084 (t, 2H), 2.784 (m, 1H), 2.086 (m, 1H), 1.904 (d, 2H), 1.302 (d, 2H), 0.366 (t, 2H), 0.197 (t, 2H).

### Step 3: tert-butyl cyclopropyl(1-(4-nitrophenyl)piperidin-4-yl)carbamate

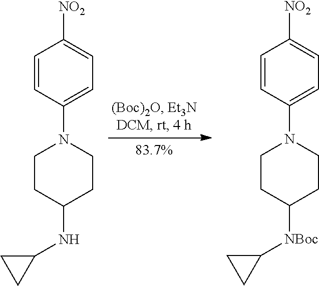 [SMILES: O=[N+]([O-])c1ccc(N2CCC(NC3CC3)CC2)cc1 ; CC(C)(C)OC(=O)N(C1CC1)C1CCN(c2ccc([N+](=O)[O-])cc2)CC1]

To a stirred solution of N-cyclopropyl-1-(4-nitrophenyl) piperidin-4-amine (1.3 g, 1.0 eq) in DCM (15 mL) in a two necked round bottomed flask under nitrogen atmosphere, triethylamine (1.2 eq) was added. To this mixture, Boc-anhydride (1.2 eq) was added and the mixture allowed to stir at rt for 4 h. After completion of starting material, the mixture was poured into water and extracted with DCM (2×50 mL). The organic layer was dried over Na₂SO₄ and concentrated. The resulting crude material was washed with pentane to obtain tert-butyl cyclopropyl(1-(4-nitrophenyl)piperidin-4-yl)carbamate (1.5 g, 83.7%) as yellow solid. 1H NMR (400 MHz, DMSO-d6): δ 8.039 (d, 2H), 7.025 (d, 2H), 4.139 (d, 2H), 3.820 (m, 1H), 3.029 (t, 2H), 2.635 (t, 1H), 1.999 (brs, 1H), 1.737 (d, 2H), 1.443 (s, 9H), 1.323 (s, 1H), 0.794 (m, 2H), 0.715 (m, 2H).

### Step 4: tert-butyl (1-(4-aminophenyl)piperidin-4-yl)(cyclopropyl)carbamate

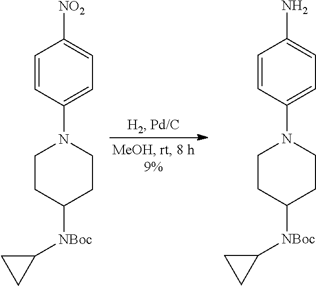 [SMILES: CC(C)(C)OC(=O)N(C1CC1)C1CCN(c2ccc([N+](=O)[O-])cc2)CC1 ; CC(C)(C)OC(=O)N(C1CC1)C1CCN(c2ccc(N)cc2)CC1]

To a stirred solution of tert-butyl cyclopropyl(1-(4-nitrophenyl)piperidin-4-yl)carbamate (1.3 g, 1.0 eq) in MeOH (20 mL) in a round bottomed flask under nitrogen atmosphere, 10% Pd/C (1.5 g, 50% wet) was added and the mixture hydrogenated with a hydrogen balloon for 8 h. After completion of starting material, the mixture was filtered through a Celite® bed and the filtrate was distilled under vacuum. The resulting crude material was purified through Combiflash® chromatography using 230-400# silica gel. The product eluted at 37% EtOAc in hexane and concentration of the pure fractions with product afforded tert-butyl (1-(4-aminophenyl)piperidin-4-yl)(cyclopropyl)carbamate as brown solid (0.1 g, 9%). 1H NMR (400 MHz, DMSO-d6): δ 6.682 (d, 2H), 6.466 (d, 2H), 4.566 (brs, 2H), 3.554 (m, 1H), 2.555 (m, 2H), 2.339 (m, 1H), 2.059 (m, 2H), 1.674 (d, 2H), 1.390 (s, 9H), 0.731 (m, 2H), 0.700 (m, 2H),

### Synthesis of 4-(4-methoxypiperidin-1-yl)aniline

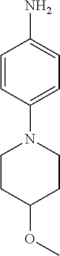

### Step 1: 4-methoxy-1-(4-nitrophenyl)piperidine

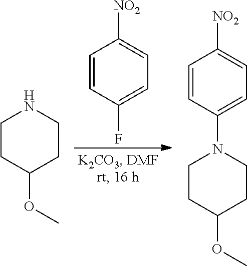

To a solution of 4-methoxypiperidine (2.0 g, 1.0 eq) in DMF (20 mL) under nitrogen atmosphere, 1-fluoro-4-nitrobenzene (2.45 g, 1.0 eq) was added and the mixture stirred at rt for 16 h. After completion of starting material on TLC, the mixture was poured into water (250 mL) and extracted with EtOAc (2×250 mL). The organic layer was dried over anhydrous Na₂SO₄, filtered and concentrated to obtain 4-methoxy-1-(4-nitrophenyl)piperidine as light yellow solid (2.5 g, 75%).

### Step 2: 4-(4-methoxypiperidin-1-yl)aniline

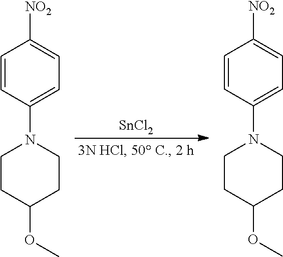 [SMILES: COC1CCN(c2ccc([N+](=O)[O-])cc2)CC1 ; "SnCl2" ; COC1CCN(c2ccc([N+](=O)[O-])cc2)CC1]

To a solution of 4-methoxy-1-(4-nitrophenyl)piperidine (2.3 g, 1.0 eq) in 3N HCl (20 mL), SnCl₂ (5.5 g, 3.0 eq) was added, and the mixture stirred at 50° C. for 2 h. After completion of starting material on TLC, the mixture was filtered through a Celite® bed and extracted with EtOAc (2×100 mL). The EtOAc layer was washed with NaHCO₃ solution, dried over anhydrous Na₂SO₄, filtered and concentrated to obtain 4-(4-methoxypiperidin-1-yl)aniline as brown colour solid (0.5 g, 24%). 1H NMR (400 MHz, DMSO-d6): δ 6.678 (d, 2H), 6.468 (d, 2H), 4.537 (s, 2H), 3.259 (s, 3H), 3.226 (m, 1H), 3.176 (m, 2H), 2.643 (m, 2H), 1.903 (m, 2H), 1.501 (m, 2H).

All the compounds listed in Table 1 were synthesized using above intermediates and by making use of general procedure as outlined in Table 1. The structure of the compounds, methods of preparation and analytical data details are given in Table 1.

Lengthy table referenced here
US20160222014A1-20160804-T00001
Please refer to the end of the specification for access instructions.

The compounds of Table 2 may be prepared in a similar manner to the methods and schemes as described for the Examples in Table 1.

TABLE 2

| | | | Calculated |
|---|---|---|---|
| | | | (M + H) m/z |
| No | Name | Structure | Value |
| 811 | N-methyl-N-(5-methyl-3-((2- ((4-(piperazin-1- yl)phenyl)amino)-7H- pyrrolo[2,3-d]pyrimidin-7- yl)methyl)pyridin-2- yl)methanesulfonamide | 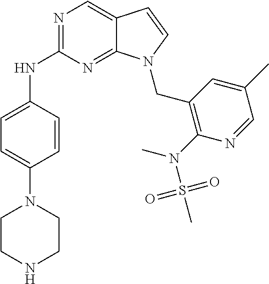 [SMILES: Cc1cnc(N(C)S(C)(=O)=O)c(Cn2ccc3cnc(Nc4ccc(N5CCNCC5)cc4)nc32)c1] | 507.22 |
| 812 | (R)-N-methyl-N-(5-methyl-3- ((2-((4-(3-methylpiperazin-1- yl)phenyl)amino)-7H- pyrrolo[2,3-d]pyrimidin-7- yl)methyl)pyridin-2- yl)methanesulfonamide | 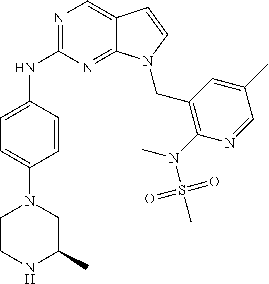 [SMILES: Cc1cnc(N(C)S(C)(=O)=O)c(Cn2ccc3cnc(Nc4ccc(N5CCN[C@H](C)C5)cc4)nc32)c1] | 521.24 |
| 813 | (S)-N-methyl-N-(5-methyl-3- ((2-((4-(3-methylpiperazin-1- yl)phenyl)amino)-7H- pyrrolo[2,3-d]pyrimidin-7- yl)methyl)pyridin-2- yl)methanesulfonamide | 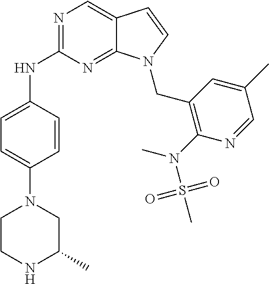 [SMILES: Cc1cnc(N(C)S(C)(=O)=O)c(Cn2ccc3cnc(Nc4ccc(N5CCN[C@@H](C)C5)cc4)nc32)c1] | 507.22 |
| 814 | N-(3-((2-((4-(4-(2- hydroxyethyl)piperazin-1- yl)phenyl)amino)-7H- pyrrolo[2,3-d]pyrimidin-7- yl)methyl)-5-methylpyridin-2- yl)-N- methylmethanesulfonamide | 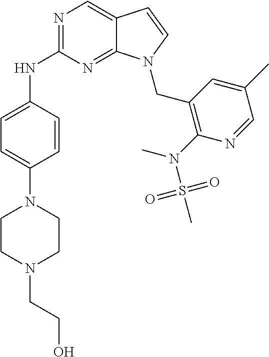 [SMILES: Cc1cnc(N(C)S(C)(=O)=O)c(Cn2ccc3cnc(Nc4ccc(N5CCN(CCO)CC5)cc4)nc32)c1] | 551.25 |
| 815 | N-(3-((2-((4-(4- (dimethylamino)piperidin-1- yl)phenyl)amino)-7H- pyrrolo[2,3-d]pyrimidin-7- yl)methyl)-5-methylpyridin-2- yl)-N- methylmethanesulfonamide | 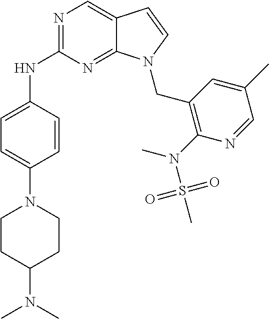 [SMILES: Cc1cnc(N(C)S(C)(=O)=O)c(Cn2ccc3cnc(Nc4ccc(N5CCC(N(C)C)CC5)cc4)nc32)c1] | 549.27 |
| 816 | 7-(isoquinolin-1-ylsulfonyl)-N- (4-(piperazin-1-yl)phenyl)-7H- pyrrolo[2,3-d]pyrimidin-2- amine | 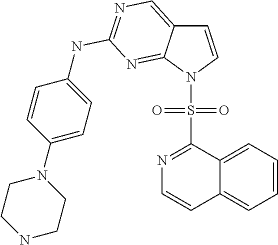 [SMILES: O=S(=O)(c1nccc2ccccc12)n1ccc2cnc(Nc3ccc(N4CCNCC4)cc3)nc21] | 486.16 |
| 817 | 7-(isoquinolin-8-ylsulfonyl)-N- (4-(piperazin-1-yl)phenyl)-7H- pyrrolo[2,3-d]pyrimidin-2- amine | 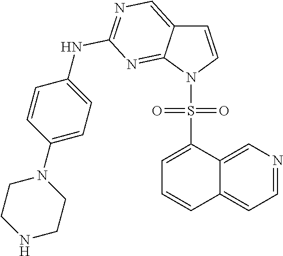 [SMILES: O=S(=O)(c1cccc2ccncc12)n1ccc2cnc(Nc3ccc(N4CCNCC4)cc3)nc21] | 486.16 |
| 818 | 7-(benzylsulfonyl)-N-(4- (piperazin-1-yl)phenyl)-7H- pyrrolo[2,3-d]pyrimidin-2- amine | 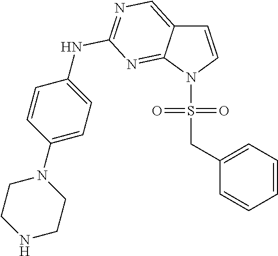 [SMILES: O=S(=O)(Cc1ccccc1)n1ccc2cnc(Nc3ccc(N4CCNCC4)cc3)nc21] | 449.17 |
| 819 | N-(4-(piperazin-1-yl)phenyl)-7- ((pyridin-2-ylmethyl)sulfonyl)- 7H-pyrrolo[2,3-d]pyrimidin-2- amine | 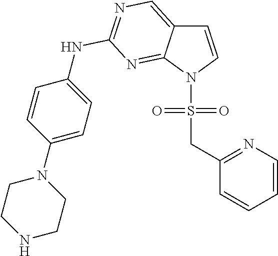 [SMILES: O=S(=O)(Cc1ccccn1)n1ccc2cnc(Nc3ccc(N4CCNCC4)cc3)nc21] | 450.16 |
| 820 | 8-((2-((4-(piperazin-1- yl)phenyl)amino)-7H- pyrrolo[2,3-d]pyrimidin-7- yl)sulfonyl)-3,4- dihydroquinolin-2(1H)-one | 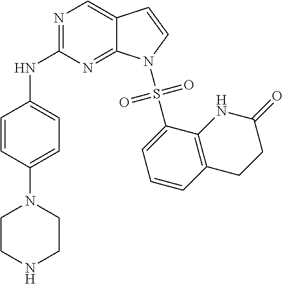 [SMILES: O=C1CCc2cccc(S(=O)(=O)n3ccc4cnc(Nc5ccc(N6CCNCC6)cc5)nc43)c2N1] | 504.17 |
| 821 | 8-((2-((4-(piperazin-1- yl)phenyl)amino)-7H- pyrrolo[2,3-d]pyrimidin-7- yl)sulfonyl)-3,4-dihydro-1H- benzo[c][1,2]thiazine 2,2- dioxide | 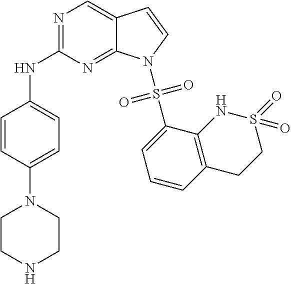 [SMILES: O=S1(=O)CCc2cccc(S(=O)(=O)n3ccc4cnc(Nc5ccc(N6CCNCC6)cc5)nc43)c2N1] | 540.14 |
| 822 | 7-((1H-indol-7-yl)sulfonyl)-N- (4-(piperazin-1-yl)phenyl)-7H- pyrrolo[2,3-d]pyrimidin-2- amine | 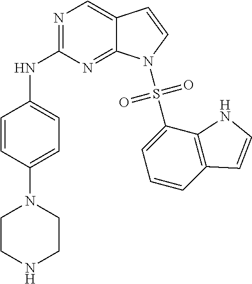 [SMILES: O=S(=O)(c1cccc2cc[nH]c12)n1ccc2cnc(Nc3ccc(N4CCNCC4)cc3)nc21] | 474.16 |
| 823 | 7-(benzo[d]thiazol-4- ylsulfonyl)-N-(4-(piperazin-1- yl)phenyl)-7H-pyrrolo[2,3- d]pyrimidin-2-amine | 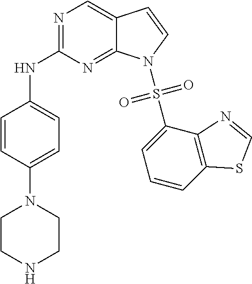 [SMILES: O=S(=O)(c1cccc2scnc12)n1ccc2cnc(Nc3ccc(N4CCNCC4)cc3)nc21] | 492.12 |
| 824 | 7-(benzo[d]thiazol-4- ylsulfonyl)-5,5-dimethyl-N-(4- (piperazin-1-yl)phenyl)-6,7- dihydro-5H-pyrrolo[2,3- d]pyrimidin-2-amine | 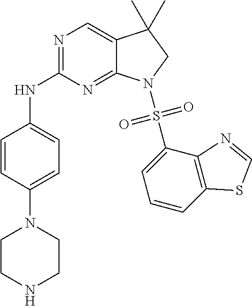 [SMILES: CC1(C)CN(S(=O)(=O)c2cccc3scnc23)c2nc(Nc3ccc(N4CCNCC4)cc3)ncc21] | 522.17 |
| 825 | 7-((2-chlorophenyl)sulfonyl)- 5,5-dimethyl-N-(4-(piperazin- 1-yl)phenyl)-6,7-dihydro-5H- pyrrolo[2,3-d]pyrimidin-2- amine | 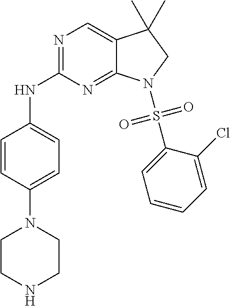 [SMILES: CC1(C)CN(S(=O)(=O)c2ccccc2Cl)c2nc(Nc3ccc(N4CCNCC4)cc3)ncc21] | 499.16 |
| 826 | N-(2-((5,5-dimethyl-2-((4- (piperazin-1-yl)phenyl)amino)- 5,6-dihydro-7H-pyrrolo[2,3- d]pyrimidin-7- yl)methyl)phenyl)-N- methylmethanesulfonamide | 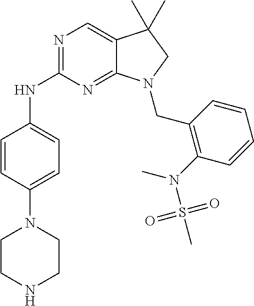 [SMILES: CN(c1ccccc1CN1CC(C)(C)c2cnc(Nc3ccc(N4CCNCC4)cc3)nc21)S(C)(=O)=O] | 522.26 |
| 827 | 5-(methoxymethyl)-N-(4- (piperazin-1-yl)phenyl)-7- (quinolin-8-ylsulfonyl)-6,7- dihydro-5H-pyrrolo[2,3- d]pyrimidin-2-amine | 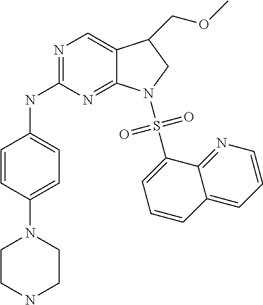 [SMILES: COCC1CN(S(=O)(=O)c2cccc3cccnc23)c2nc(Nc3ccc(N4CCNCC4)cc3)ncc21] | 532.21 |
| 828 | 5-methyl-N-(4-(piperazin-1- yl)phenyl)-7-(quinolin-8- ylsulfonyl)-6,7-dihydro-5H- pyrrolo[2,3-d]pyrimidin-2- amine | 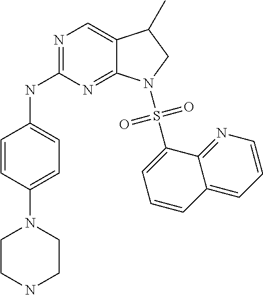 [SMILES: CC1CN(S(=O)(=O)c2cccc3cccnc23)c2nc(Nc3ccc(N4CCNCC4)cc3)ncc21] | 502.19 |
| 829 | N-(2-((2-((2-methoxy-4- (piperidin-4-yl)phenyl)amino)- 7H-pyrrolo[2,3-d]pyrimidin-7- yl)methyl)phenyl)-N- methylmethanesulfonamide | 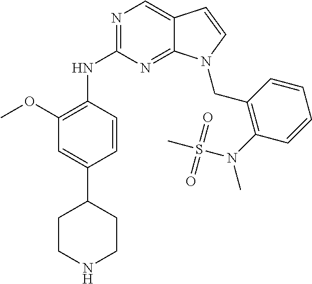 [SMILES: COc1cc(C2CCNCC2)ccc1Nc1ncc2ccn(Cc3ccccc3N(C)S(C)(=O)=O)c2n1] | 521.23 |
| 830 | N-(2-((2-((2-methoxy-4-(1- methylpiperidin-4- yl)phenyl)amino)-7H- pyrrolo[2,3-d]pyrimidin-7- yl)methyl)phenyl)-N- methylmethanesulfonamide | 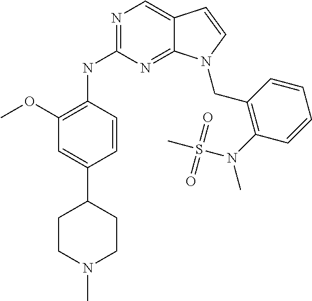 [SMILES: COc1cc(C2CCN(C)CC2)ccc1Nc1ncc2ccn(Cc3ccccc3N(C)S(C)(=O)=O)c2n1] | 535.24 |
| 831 | N-(2-((2-((2-fluoro-4- (piperidin-4-yl)phenyl)amino)- 7H-pyrrolo[2,3-d]pyrimidin-7- yl)methyl)phenyl)-N- methylmethanesulfonamide | 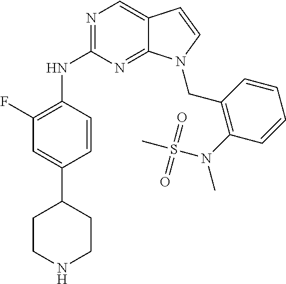 [SMILES: CN(c1ccccc1Cn1ccc2cnc(Nc3ccc(C4CCNCC4)cc3F)nc21)S(C)(=O)=O] | 509.21 |
| 832 | (2-((2-((4-(piperazin-1- yl)phenyl)amino)-7H- pyrrolo[2,3-d]pyrimidin-7- yl)methyl)phenyl)methanesulfonamide | 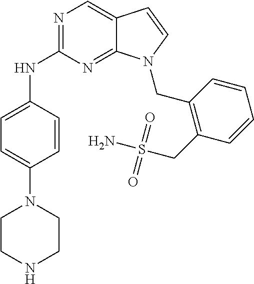 [SMILES: NS(=O)(=O)Cc1ccccc1Cn1ccc2cnc(Nc3ccc(N4CCNCC4)cc3)nc21] | 478.19 |
| 833 | N-methyl-N-(2-(2-(2-((4- (piperazin-1-yl)phenyl)amino)- 7H-pyrrolo[2,3-d]pyrimidin-7- yl)ethyl)phenyl)methanesulfonamide | 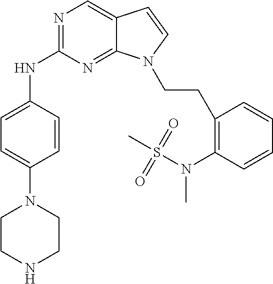 [SMILES: CN(c1ccccc1CCn1ccc2cnc(Nc3ccc(N4CCNCC4)cc3)nc21)S(C)(=O)=O] | 506.23 |
| 834 | 2-((6-((4-(2,5- dimethylpiperazin-1- yl)phenyl)amino)-1H- pyrrolo[3,2-c]pyridin-1- yl)sulfonyl)benzonitrile | 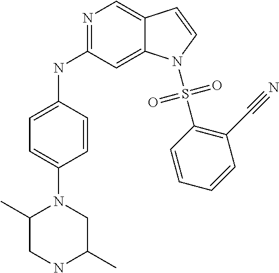 [SMILES: CC1CN(c2ccc(Nc3cc4c(ccn4S(=O)(=O)c4ccccc4C#N)cn3)cc2)C(C)CN1] | 487.18 |
| 835 | N-methyl-N-(2-((2-((4- (piperazin-1-yl)phenyl)amino)- 7H-pyrrolo[2,3-d]pyrimidin-7- yl)methyl)phenyl)ethanesulfonamide | 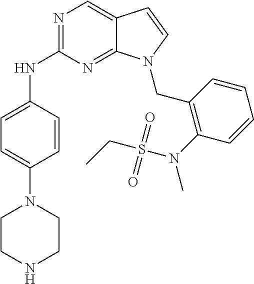 [SMILES: CCS(=O)(=O)N(C)c1ccccc1Cn1ccc2cnc(Nc3ccc(N4CCNCC4)cc3)nc21] | 506.23 |
| 836 | N-methyl-N-(2-((2-((4- (piperazin-1-yl)phenyl)amino)- 7H-pyrrolo[2,3-d]pyrimidin-7- yl)methyl)phenyl)propane-2- sulfonamide | 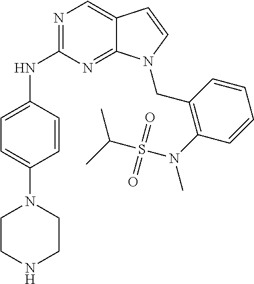 [SMILES: CC(C)S(=O)(=O)N(C)c1ccccc1Cn1ccc2cnc(Nc3ccc(N4CCNCC4)cc3)nc21] | 520.24 |
| 837 | (S)-N-methyl-N-(3-((2-((4-(3- methylpiperazin-1- yl)phenyl)amino)-7H- pyrrolo[2,3-d]pyrimidin-7- yl)methyl)pyridin-4- yl)methanesulfonamide | 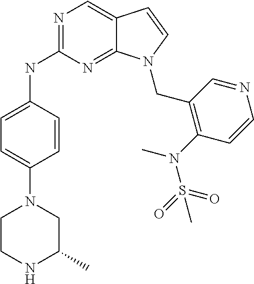 [SMILES: C[C@H]1CN(c2ccc(Nc3ncc4ccn(Cc5cnccc5N(C)S(C)(=O)=O)c4n3)cc2)CCN1] | 507.22 |
| 838 | (S)-N-methyl-N-(4-((2-((4-(3- methylpiperazin-1- yl)phenyl)amino)-7H- pyrrolo[2,3-d]pyrimidin-7- yl)methyl)pyridin-3- yl)methanesulfonamide | 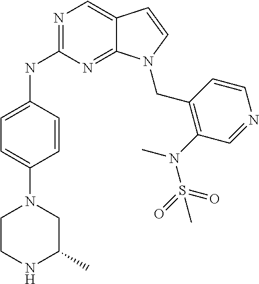 [SMILES: C[C@H]1CN(c2ccc(Nc3ncc4ccn(Cc5ccncc5N(C)S(C)(=O)=O)c4n3)cc2)CCN1] | 507.22 |
| 839 | N-(3-((2-((4-(piperazin-1- yl)phenyl)amino)-7H- pyrrolo[2,3-d]pyrimidin-7- yl)methyl)pyridin-2- yl)methanesulfonamide | 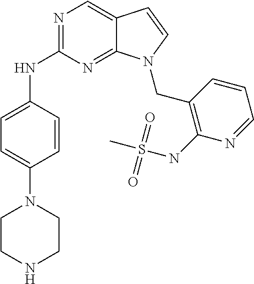 [SMILES: CS(=O)(=O)Nc1ncccc1Cn1ccc2cnc(Nc3ccc(N4CCNCC4)cc3)nc21] | 479.19 |
| 840 | (R)-N-(3-((2-((4-(3- methylpiperazin-1- yl)phenyl)amino)-7H- pyrrolo[2,3-d]pyrimidin-7- yl)methyl)pyridin-2- yl)methanesulfonamide | 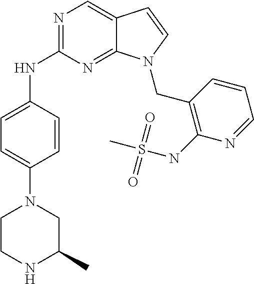 [SMILES: C[C@@H]1CN(c2ccc(Nc3ncc4ccn(Cc5cccnc5NS(C)(=O)=O)c4n3)cc2)CCN1] | 493.21 |
| 841 | N-(3-((2-((2-oxoindolin-5- yl)amino)-7H-pyrrolo[2,3- d]pyrimidin-7- yl)methyl)pyridin-2- yl)methanesulfonamide | 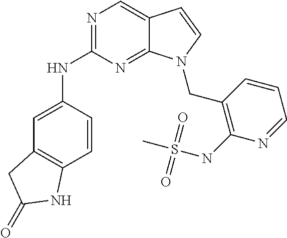 [SMILES: CS(=O)(=O)Nc1ncccc1Cn1ccc2cnc(Nc3ccc4c(c3)CC(=O)N4)nc21] | 450.13 |
| 842 | N-(3-((2-((4-(piperazin-1- yl)phenyl)amino)-7H- pyrrolo[2,3-d]pyrimidin-7- yl)methyl)pyridin-2- yl)cyclopropanesulfonamide | 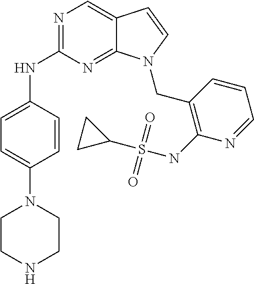 [SMILES: O=S(=O)(Nc1ncccc1Cn1ccc2cnc(Nc3ccc(N4CCNCC4)cc3)nc21)C1CC1] | 505.21 |
| 843 | (S)-N-(3-((2-((4-(3- methylpiperazin-1- yl)phenyl)amino)-7H- pyrrolo[2,3-d]pyrimidin-7- yl)methyl)pyridin-2- yl)methanesulfonamide | 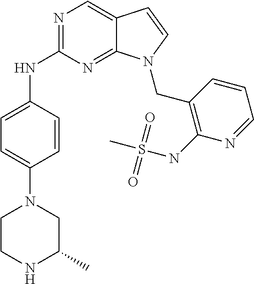 [SMILES: C[C@H]1CN(c2ccc(Nc3ncc4ccn(Cc5cccnc5NS(C)(=O)=O)c4n3)cc2)CCN1] | 493.21 |
| 844 | (S)-N-methyl-N-(2-((2-((4-(2- methylpiperazin-1- yl)phenyl)amino)-7H- pyrrolo[2,3-d]pyrimidin-7- yl)methyl)pyridin-3- yl)methanesulfonamide | 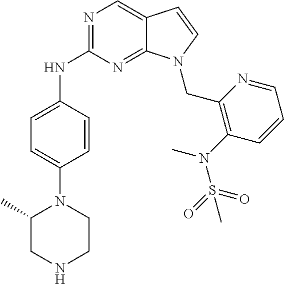 [SMILES: C[C@H]1CNCCN1c1ccc(Nc2ncc3ccn(Cc4ncccc4N(C)S(C)(=O)=O)c3n2)cc1] | 507.22 |
| 845 | (S)-N-methyl-N-(3-((2-((4-(2- methylpiperazin-1- yl)phenyl)amino)-7H- pyrrolo[2,3-d]pyrimidin-7- yl)methyl)pyridin-4- yl)methanesulfonamide | 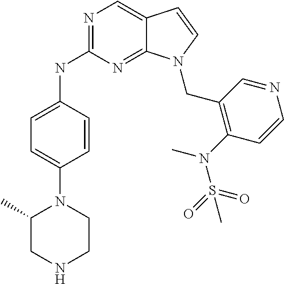 [SMILES: C[C@H]1CNCCN1c1ccc(Nc2ncc3ccn(Cc4cnccc4N(C)S(C)(=O)=O)c3n2)cc1] | 507.22 |
| 846 | (S)-N-methyl-N-(4-((2-((4-(2- methylpiperazin-1- yl)phenyl)amino)-7H- pyrrolo[2,3-d]pyrimidin-7- yl)methyl)pyridin-3- yl)methanesulfonamide | 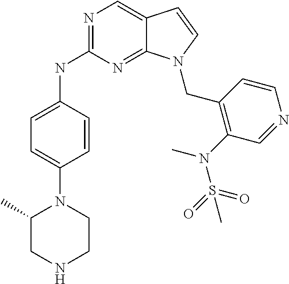 [SMILES: C[C@H]1CNCCN1c1ccc(Nc2ncc3ccn(Cc4ccncc4N(C)S(C)(=O)=O)c3n2)cc1] | 507.22 |
| 847 | (S)-N-methyl-N-(3-((2-((4-(2- methylpiperazin-1- yl)phenyl)amino)-7H- pyrrolo[2,3-d]pyrimidin-7- yl)methyl)pyridin-2- yl)cyclopropanesulfonamide | 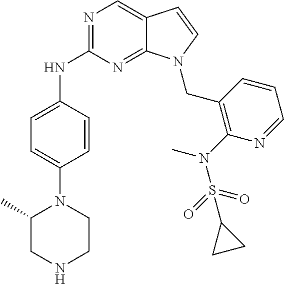 [SMILES: C[C@H]1CNCCN1c1ccc(Nc2ncc3ccn(Cc4cccnc4N(C)S(=O)(=O)C4CC4)c3n2)cc1] | 533.24 |
| 848 | N-(3-((2-((4-(2,2- dimethylpiperazin-1- yl)phenyl)amino)-7H- pyrrolo[2,3-d]pyrimidin-7- yl)methyl)pyridin-2-yl)-N- methylmethanesulfonamide | 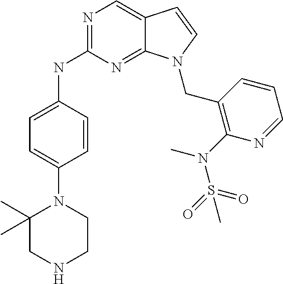 [SMILES: CN(c1ncccc1Cn1ccc2cnc(Nc3ccc(N4CCNCC4(C)C)cc3)nc21)S(C)(=O)=O] | 521.24 |
| 849 | (R)-N-(3-((2-((4-(2- (hydroxymethyl)piperazin-1- yl)phenyl)amino)-7H- pyrrolo[2,3-d]pyrimidin-7- yl)methyl)pyridin-2-yl)-N- methylmethanesulfonamide | 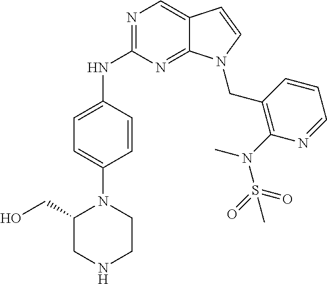 [SMILES: CN(c1ncccc1Cn1ccc2cnc(Nc3ccc(N4CCNC[C@@H]4CO)cc3)nc21)S(C)(=O)=O] | 523.22 |
| 850 | (S)-N-(3-((2-((4-(2- ethylpiperazin-1- yl)phenyl)amino)-7H- pyrrolo[2,3-d]pyrimidin-7- yl)methyl)pyridin-2-yl)-N- methylmethanesulfonamide | 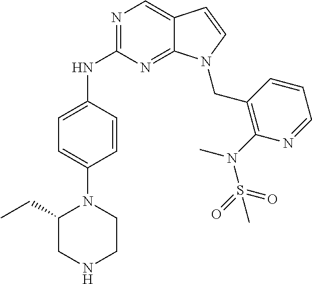 [SMILES: CC[C@H]1CNCCN1c1ccc(Nc2ncc3ccn(Cc4cccnc4N(C)S(C)(=O)=O)c3n2)cc1] | 521.24 |
| 851 | (S)-N-(3-((2-((3-fluoro-4-(2- methylpiperazin-1- yl)phenyl)amino)-7H- pyrrolo[2,3-d]pyrimidin-7- yl)methyl)pyridin-2-yl)-N- methylmethanesulfonamide | 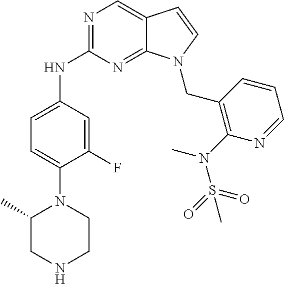 [SMILES: C[C@H]1CNCCN1c1ccc(Nc2ncc3ccn(Cc4cccnc4N(C)S(C)(=O)=O)c3n2)cc1F] | 525.21 |
| 852 | N-methyl-N-(3-((2-((4-((2- (piperazin-1- yl)ethyl)amino)phenyl)amino)- 7H-pyrrolo[2,3-d]pyrimidin-7- yl)methyl)pyridin-2- yl)methanesulfonamide | 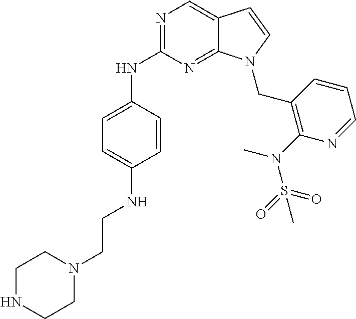 [SMILES: CN(c1ncccc1Cn1ccc2cnc(Nc3ccc(NCCN4CCNCC4)cc3)nc21)S(C)(=O)=O] | 536.25 |
| 853 | N-(2-hydroxyethyl)-N-(2-((2- ((2-oxoindolin-5-yl)amino)- 7H-pyrrolo[2,3-d]pyrimidin-7- yl)methyl)phenyl) methanesulfonamide | 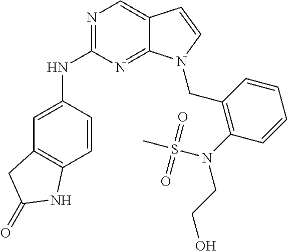 [SMILES: CS(=O)(=O)N(CCO)c1ccccc1Cn1ccc2cnc(Nc3ccc4c(c3)CC(=O)N4)nc21] | 493.16 |
| 854 | N-(2-hydroxyethyl)-N-(3-((2- ((4-(piperazin-1- yl)phenyl)amino)-7H- pyrrolo[2,3-d]pyrimidin-7- yl)methyl)pyridin-2- yl)methanesulfonamide | 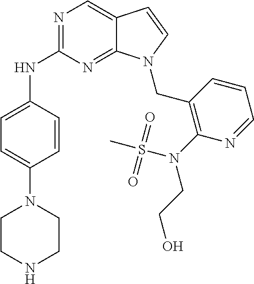 [SMILES: CS(=O)(=O)N(CCO)c1ncccc1Cn1ccc2cnc(Nc3ccc(N4CCNCC4)cc3)nc21] | 523.22 |
| 855 | (R)-N-(2-hydroxyethyl)-N-(3- ((2-((4-(3-methylpiperazin-1- yl)phenyl)amino)-7H- pyrrolo[2,3-d]pyrimidin-7- yl)methyl)pyridin-2- yl)methanesulfonamide | 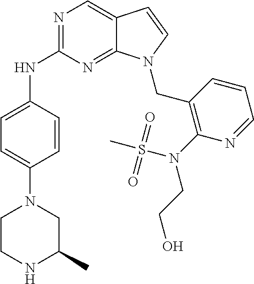 [SMILES: C[C@@H]1CN(c2ccc(Nc3ncc4ccn(Cc5cccnc5N(CCO)S(C)(=O)=O)c4n3)cc2)CCN1] | 537.23 |
| 856 | (R)-N-(1-(hydroxymethyl) cyclopropyl)-N-(2-((2-((4-(3- methylpiperazin-1- yl)phenyl)amino)-7H- pyrrolo[2,3-d]pyrimidin-7- yl)methyl)phenyl)methanesulfonamide | 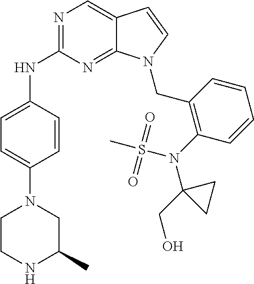 [SMILES: C[C@@H]1CN(c2ccc(Nc3ncc4ccn(Cc5ccccc5N(C5(CO)CC5)S(C)(=O)=O)c4n3)cc2)CCN1] | 562.25 |
| 857 | (R)-N-cyclopropyl-N-(3-((2- ((4-(3-methylpiperazin-1- yl)phenyl)amino)-7H- pyrrolo[2,3-d]pyrimidin-7- yl)methyl)pyridin-2- yl)methanesulfonamide | 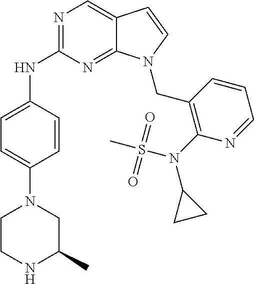 [SMILES: C[C@@H]1CN(c2ccc(Nc3ncc4ccn(Cc5cccnc5N(C5CC5)S(C)(=O)=O)c4n3)cc2)CCN1] | 532.24 |
| 858 | N-methyl-N-(2-((2-((4-(3- (pyrrolidin-1-yl)azetidin-1- yl)phenyl)amino)-7H- pyrrolo[2,3-d]pyrimidin-7- yl)methyl)phenyl) methanesulfonamide | 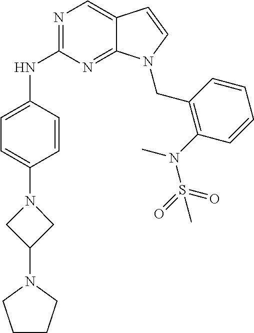 [SMILES: CN(c1ccccc1Cn1ccc2cnc(Nc3ccc(N4CC(N5CCCC5)C4)cc3)nc21)S(C)(=O)=O] | 532.24 |
| 859 | N-methyl-N-(2-((2-((4-(3- (piperazin-1-yl)azetidin-1- yl)phenyl)amino)-7H- pyrrolo[2,3-d]pyrimidin-7- yl)methyl)phenyl) methanesulfonamide | 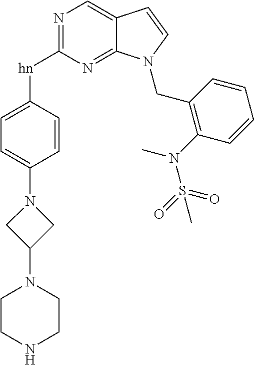 | 547.25 |
| 860 | N-(2-((2-((1-(2-hydroxyethyl)- 2-oxo-1,2,3,4- tetrahydroquinolin-6- yl)amino)-7H-pyrrolo[2,3- d]pyrimidin-7- yl)methyl)phenyl) methanesulfonamide | 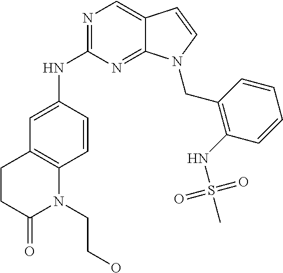 [SMILES: CS(=O)(=O)Nc1ccccc1Cn1ccc2cnc(Nc3ccc4c(c3)CCC(=O)N4CCO)nc21] | 507.17 |
| 861 | N-(2-((2-((1-(2- (dimethylamino)ethyl)-2-oxo- 1,2,3,4-tetrahydroquinolin-6- yl)amino)-7H-pyrrolo[2,3- d]pyrimidin-7-yl)methyl) phenyl)methanesulfonamide | 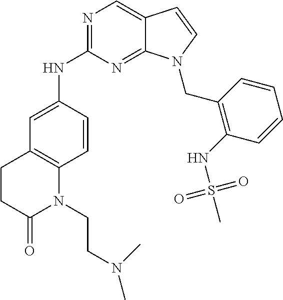 [SMILES: CN(C)CCN1C(=O)CCc2cc(Nc3ncc4ccn(Cc5ccccc5NS(C)(=O)=O)c4n3)ccc21] | 534.22 |
| 862 | N-(3-((2-((4-(4,7- diazaspiro[2.5]octan-7- yl)phenyl)amino)-7H- pyrrolo[2,3-d]pyrimidin-7- yl)methyl)pyridin-2-yl)-N- methylmethanesulfonamide | 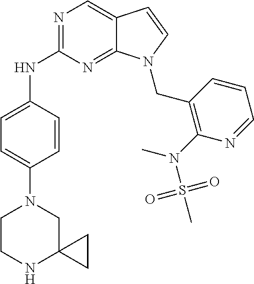 [SMILES: CN(c1ncccc1Cn1ccc2cnc(Nc3ccc(N4CCNC5(CC5)C4)cc3)nc21)S(C)(=O)=O] | 519.22 |
| 863 | N-methyl-N-(2-((2-((4- (morpholin-3- yl)phenyl)amino)-7H- pyrrolo[2,3-d]pyrimidin-7- yl)methyl)phenyl)methanesulfonamide | 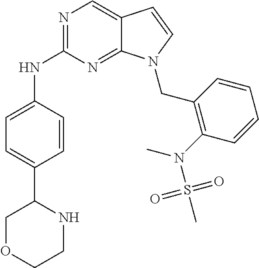 [SMILES: CN(c1ccccc1Cn1ccc2cnc(Nc3ccc(C4COCCN4)cc3)nc21)S(C)(=O)=O] | 493.19 |
| 864 | N-(4-(4-(dimethylamino)cyclohexyl) phenyl)-5,5-dimethyl-7-(quinolin- 8-ylsulfonyl)-6,7-dihydro-5H- pyrrolo[2,3-d]pyrimidin-2- amine | 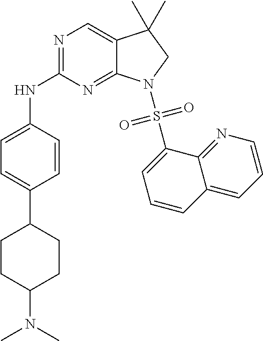 [SMILES: CN(C)C1CCC(c2ccc(Nc3ncc4c(n3)N(S(=O)(=O)c3cccc5cccnc35)CC4(C)C)cc2)CC1] | 557.26 |
| 865 | N-methyl-N-(2-((2-((4-((S)-3- methylpiperazin-1- yl)phenyl)amino)-7H- pyrrolo[2,3-d]pyrimidin-7- yl)methyl)phenyl) methanesulfonimidamide | 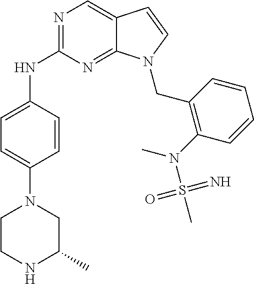 [SMILES: C[C@H]1CN(c2ccc(Nc3ncc4ccn(Cc5ccccc5N(C)S(C)(=N)=O)c4n3)cc2)CCN1] | 505.24 |
| 866 | N-methyl-N-(3-((2-((4-((S)-3- methylpiperazin-1- yl)phenyl)amino)-7H- pyrrolo[2,3-d]pyrimidin-7- yl)methyl)pyridin-2- yl)methanesulfonimidamide | 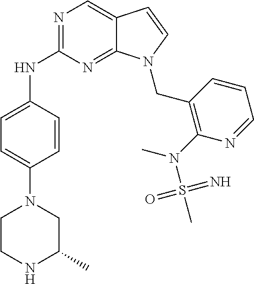 [SMILES: C[C@H]1CN(c2ccc(Nc3ncc4ccn(Cc5cccnc5N(C)S(C)(=N)=O)c4n3)cc2)CCN1] | 506.24 |
| 867 | N-(2-cyano-6-((2-((4- (piperazin-1-yl)phenyl)amino)- 7H-pyrrolo[2,3-d]pyrimidin-7- yl)methyl)phenyl)-N- methylmethanesulfonamide | 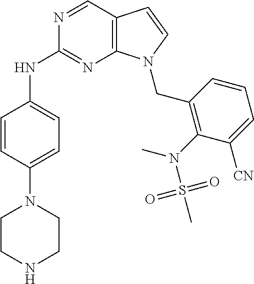 [SMILES: CN(c1c(C#N)cccc1Cn1ccc2cnc(Nc3ccc(N4CCNCC4)cc3)nc21)S(C)(=O)=O] | 517.21 |
| 868 | (R)-N-(2-cyano-6-((2-((4-(3- methylpiperazin-1- yl)phenyl)amino)-7H- pyrrolo[2,3-d]pyrimidin-7- yl)methyl)phenyl)-N- methylmethanesulfonamide | 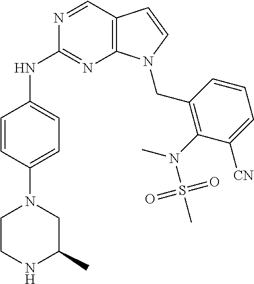 [SMILES: C[C@@H]1CN(c2ccc(Nc3ncc4ccn(Cc5cccc(C#N)c5N(C)S(C)(=O)=O)c4n3)cc2)CCN1] | 531.22 |
| 869 | (S)-N-(2-cyano-6-((2-((4-(3- methylpiperazin-1- yl)phenyl)amino)-7H- pyrrolo[2,3-d]pyrimidin-7- yl)methyl)phenyl)-N- methylmethanesulfonamide | 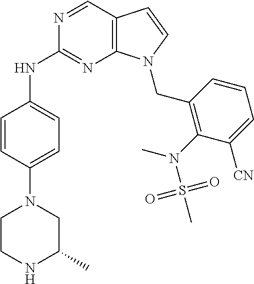 [SMILES: C[C@H]1CN(c2ccc(Nc3ncc4ccn(Cc5cccc(C#N)c5N(C)S(C)(=O)=O)c4n3)cc2)CCN1] | 531.22 |
| 870 | N,N-dimethyl-3-((2-((4- (piperazin-1-yl)phenyl)amino)- 7H-pyrrolo[2,3-d]pyrimidin-7- yl)methyl)picolinamide | 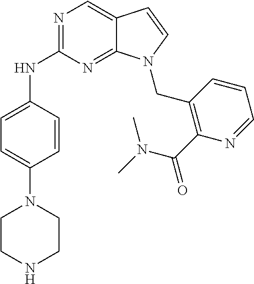 [SMILES: CN(C)C(=O)c1ncccc1Cn1ccc2cnc(Nc3ccc(N4CCNCC4)cc3)nc21] | 457.24 |
| 871 | (R)-N,N-dimethyl-3-((2-((4-(3- methylpiperazin-1- yl)phenyl)amino)-7H- pyrrolo[2,3-d]pyrimidin-7- yl)methyl)picolinamide | 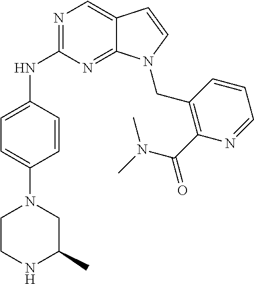 [SMILES: C[C@@H]1CN(c2ccc(Nc3ncc4ccn(Cc5cccnc5C(=O)N(C)C)c4n3)cc2)CCN1] | 471.25 |
| 872 | N-(2-(5,5-dimethyl-2-((3- (1,2,3,6-tetrahydropyridin-4- yl)-1H-indol-5-yl)amino)-5,6- dihydro-7H-pyrrolo[2,3- d]pyrimidin-7-yl)phenyl)-N- methylmethanesulfonamide | 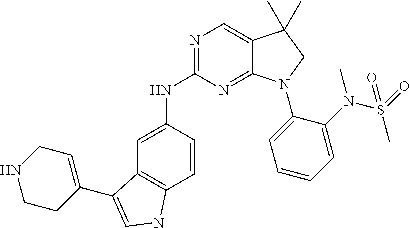 [SMILES: CN(c1ccccc1N1CC(C)(C)c2cnc(Nc3ccc4[nH]cc(C5=CCNCC5)c4c3)nc21)S(C)(=O)=O] | 544.24 |
| 873 | N-(2-(5,5-dimethyl-2-((3- (1,2,3,6-tetrahydropyridin-4- yl)-1H-indol-5-yl)amino)-5,6- dihydro-7H-pyrrolo[2,3- d]pyrimidin-7-yl)phenyl)-N- methylacetamide | 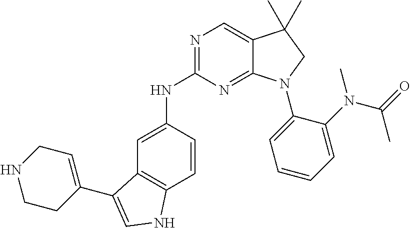 [SMILES: CC(=O)N(C)c1ccccc1N1CC(C)(C)c2cnc(Nc3ccc4[nH]cc(C5=CCNCC5)c4c3)nc21] | 508.27 |
| 874 | (S)-N-methyl-N-(2-(2-(2-((4- (3-methylpiperazin-1- yl)phenyl)amino)-7H- pyrrolo[2,3-d]pyrimidin- 7-yl)propan-2- yl)phenyl)methanesulfonamide | 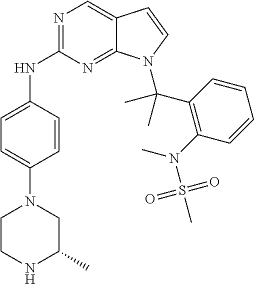 [SMILES: C[C@H]1CN(c2ccc(Nc3ncc4ccn(C(C)(C)c5ccccc5N(C)S(C)(=O)=O)c4n3)cc2)CCN1] | 534.26 |
| 875 | N-(2-(2-(2-((4-(4- (dimethylamino)piperidin-1- yl)phenyl)amino)-7H- pyrrolo[2,3-d]pyrimidin-7- yl)propan-2-yl)phenyl)-N- methylmethanesulfonamide | 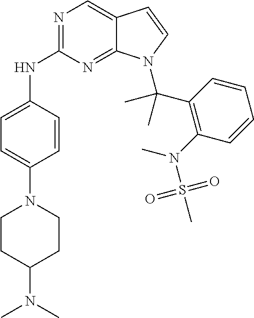 [SMILES: CN(C)C1CCN(c2ccc(Nc3ncc4ccn(C(C)(C)c5ccccc5N(C)S(C)(=O)=O)c4n3)cc2)CC1] | 562.29 |
| 876 | (S)-N-methyl-N-(3-((2-((4-(3- methylpiperazin-1- yl)phenyl)amino)-7H- pyrrolo[2,3-d]pyrimidin-7- yl)methyl)pyridin-2- yl)acetamide | 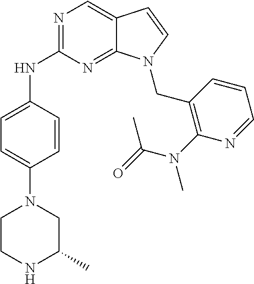 [SMILES: CC(=O)N(C)c1ncccc1Cn1ccc2cnc(Nc3ccc(N4CCN[C@@H](C)C4)cc3)nc21] | 471.25 |
| 877 | (R)-N-(2-((5-cyano-6-((4-(3- methylpiperazin-1- yl)phenyl)amino)-1H- pyrrolo[2,3-b]pyridin-1- yl)methyl)phenyl)-N- methylmethanesulfonamide | 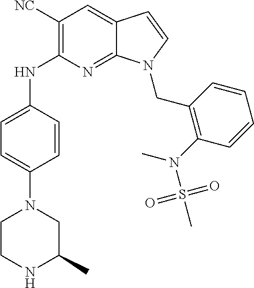 [SMILES: C[C@@H]1CN(c2ccc(Nc3nc4c(ccn4Cc4ccccc4N(C)S(C)(=O)=O)cc3C#N)cc2)CCN1] | 430.23 |
| 878 | (S)-N-(2-((5-cyano-6-((4-(3- methylpiperazin-1- yl)phenyl)amino)-1H- pyrrolo[2,3-b]pyridin-1- yl)methyl)phenyl)-N- methylmethanesulfonamide | 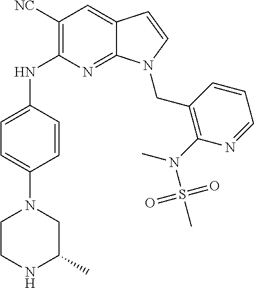 [SMILES: C[C@H]1CN(c2ccc(Nc3nc4c(ccn4Cc4cccnc4N(C)S(C)(=O)=O)cc3C#N)cc2)CCN1] | 430.23 |
| 879 | (R)-N-methyl-N-(2-((5-((4-(3- methylpiperazin-1- yl)phenyl)amino)-3H- [1,2,3]triazolo[4,5-d] pyrimidin-3-yl)methyl) phenyl)methanesulfonamide | 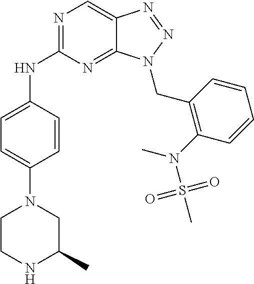 [SMILES: C[C@@H]1CN(c2ccc(Nc3ncc4nnn(Cc5ccccc5N(C)S(C)(=O)=O)c4n3)cc2)CCN1] | 508.22 |
| 880 | (S)-N-(4-chloro-2-((2-((3- methyl-4-(3-methylpiperazin-1- yl)phenyl)amino)-7H- pyrrolo[2,3-d]pyrimidin-7- yl)methyl)phenyl)-N- methylmethanesulfonamide | 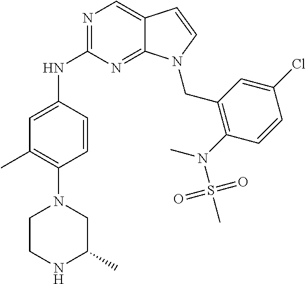 [SMILES: Cc1cc(Nc2ncc3ccn(Cc4cc(Cl)ccc4N(C)S(C)(=O)=O)c3n2)ccc1N1CCN[C@@H](C)C1] | 555.18 |
| 881 | 1,3-dimethyl-1-(3-((2-((4- (piperazin-1-yl)phenyl)amino)- 7H-pyrrolo[2,3-d]pyrimidin-7- yl)methyl)pyridin-2-yl)urea | 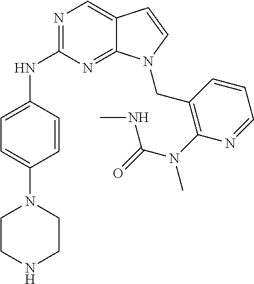 [SMILES: CNC(=O)N(C)c1ncccc1Cn1ccc2cnc(Nc3ccc(N4CCNCC4)cc3)nc21] | 472.25 |
| 882 | (S)-1,3-dimethyl-1-(3-((2-((4- (3-methylpiperazin-1- yl)phenyl)amino)-7H- pyrrolo[2,3-d]pyrimidin-7- yl)methyl)pyridin-2-yl)urea | 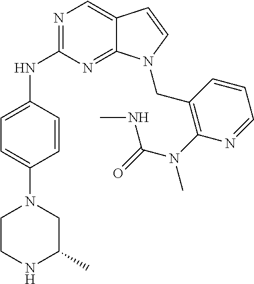 [SMILES: CNC(=O)N(C)c1ncccc1Cn1ccc2cnc(Nc3ccc(N4CCN[C@@H](C)C4)cc3)nc21] | 486.27 |
| 883 | (R)-1,3-dimethyl-1-(2-((2-((4- (3-methylpiperazin-1- yl)phenyl)amino)-7H- pyrrolo[2,3-d]pyrimidin-7- yl)methyl)phenyl)urea | 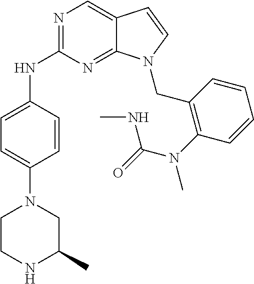 [SMILES: CNC(=O)N(C)c1ccccc1Cn1ccc2cnc(Nc3ccc(N4CCN[C@H](C)C4)cc3)nc21] | 485.27 |
| 884 | 1-(2-((2-((4-(4- (dimethylamino)piperidin-1- yl)phenyl)amino)-7H- pyrrolo[2,3-d]pyrimidin-7- yl)methyl)phenyl)-1,3- dimethylurea | 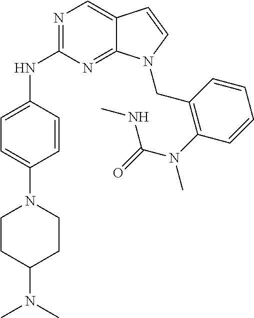 [SMILES: CNC(=O)N(C)c1ccccc1Cn1ccc2cnc(Nc3ccc(N4CCC(N(C)C)CC4)cc3)nc21] | 513.30 |
| 885 | N-(4-chloro-2-((2-((3-methyl- 4-(piperidin-4- yl)phenyl)amino)-7H- pyrrolo[2,3-d]pyrimidin-7- yl)methyl)phenyl)-N- methylmethanesulfonamide | 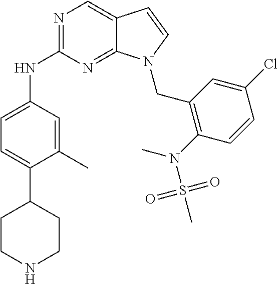 [SMILES: Cc1cc(Nc2ncc3ccn(Cc4cc(Cl)ccc4N(C)S(C)(=O)=O)c3n2)ccc1C1CCNCC1] | 540.19 |
| 886 | (S)-N-methyl-N-(3-((2-((4-(2- methylpiperidin-4- yl)phenyl)amino)-7H- pyrrolo[2,3-d]pyrimidin-7- yl)methyl)pyridin-2- yl)methanesulfonamide | 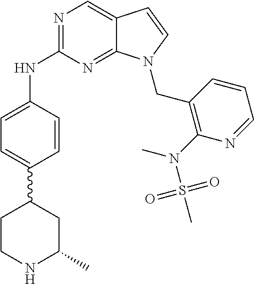 [SMILES: C[C@H]1CC(c2ccc(Nc3ncc4ccn(Cc5cccnc5N(C)S(C)(=O)=O)c4n3)cc2)CCN1] | 505.24 |
| 889 | N-(2-((2-((4-(1-(2- (dimethylamino)ethyl) piperidin-4-yl)-3-methylphenyl) amino)-7H-pyrrolo[2,3-d] pyrimidin-7-yl)methyl)phenyl)-N- methylmethanesulfonamide | 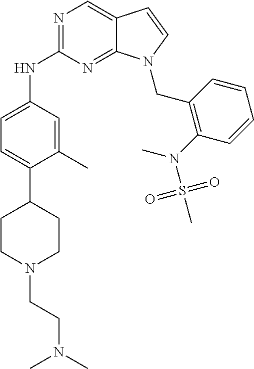 [SMILES: Cc1cc(Nc2ncc3ccn(Cc4ccccc4N(C)S(C)(=O)=O)c3n2)ccc1C1CCN(CCN(C)C)CC1] | 576.30 |
| 890 | 7-(2-(isopropylsulfonyl)phenyl)- 5,5-dimethyl-N-(3-methyl-4- (piperidin-4-yl)phenyl)-6,7- dihydro-5H-pyrrolo[2,3- d]pyrimidin-2-amine | 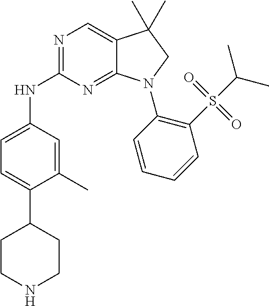 [SMILES: Cc1cc(Nc2ncc3c(n2)N(c2ccccc2S(=O)(=O)C(C)C)CC3(C)C)ccc1C1CCNCC1] | 520.27 |
| 891 | 2-(5,5-dimethyl-2-((3-methyl- 4-(piperidin-4- yl)phenyl)amino)-5,6-dihydro- 7H-pyrrolo[2,3-d]pyrimidin- 7-yl)-N,N- dimethylbenzenesulfonamide | 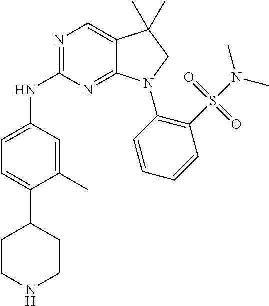 [SMILES: Cc1cc(Nc2ncc3c(n2)N(c2ccccc2S(=O)(=O)N(C)C)CC3(C)C)ccc1C1CCNCC1] | 521.26 |
| 893 | (R)-N-methyl-N-(2-((2-((4-(3- methylpiperazin-1- yl)phenyl)amino)-7H- pyrrolo[2,3-d]pyrimidin-7- yl)methyl)-4-(trifluoromethyl) phenyl)methanesulfonamide | 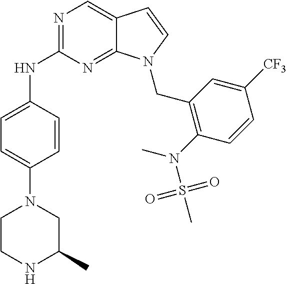 [SMILES: C[C@@H]1CN(c2ccc(Nc3ncc4ccn(Cc5cc(C(F)(F)F)ccc5N(C)S(C)(=O)=O)c4n3)cc2)CCN1] | 574.22 |
| 894 | N-methyl-N-(2-((2-((4- (piperidin-4-yl)phenyl)amino)- 7H-pyrrolo[2,3-d]pyrimidin- 7-yl)methyl)-4- (trifluoromethyl)phenyl) methanesulfonamide | 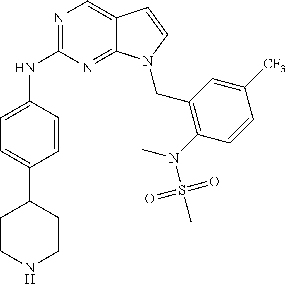 [SMILES: CN(c1ccc(C(F)(F)F)cc1Cn1ccc2cnc(Nc3ccc(C4CCNCC4)cc3)nc21)S(C)(=O)=O] | 559.21 |
| 895 | N-(2-((2-((4-(4-(2- hydroxyethyl)piperazin-1- yl)phenyl)amino)-7H- pyrrolo[2,3-d]pyrimidin-7- yl)methyl)-4- (trifluoromethyl)phenyl)-N- methylmethanesulfonamide | 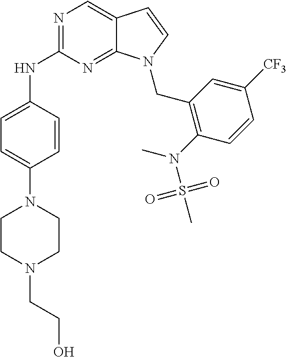 [SMILES: CN(c1ccc(C(F)(F)F)cc1Cn1ccc2cnc(Nc3ccc(N4CCN(CCO)CC4)cc3)nc21)S(C)(=O)=O] | 604.23 |
| 896 | N-(2-((2-((4-(4- (dimethylamino)piperidin-1- yl)phenyl)amino)-7H- pyrrolo[2,3-d]pyrimidin-7- yl)methyl)-4- (trifluoromethyl)phenyl)-N- methylmethanesulfonamide | 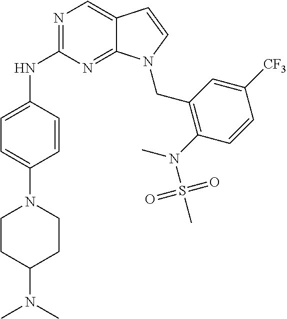 [SMILES: CN(C)C1CCN(c2ccc(Nc3ncc4ccn(Cc5cc(C(F)(F)F)ccc5N(C)S(C)(=O)=O)c4n3)cc2)CC1] | 602.22 |
| 897 | N-(4-chloro-2-((2-((4- (piperazin-1-yl)phenyl)amino)- 7H-pyrrolo[2,3-d]pyrimidin-7- yl)methyl)phenyl)-N- methylmethanesulfonamide | 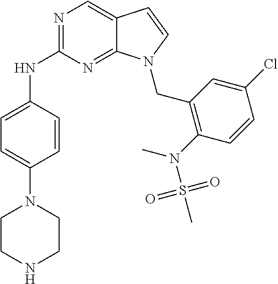 [SMILES: CN(c1ccc(Cl)cc1Cn1ccc2cnc(Nc3ccc(N4CCNCC4)cc3)nc21)S(C)(=O)=O] | 527.17 |
| 898 | (R)-N-(4-chloro-2-((2-((4-(3- methylpiperazin-1- yl)phenyl)amino)-7H- pyrrolo[2,3-d]pyrimidin-7- yl)methyl)phenyl)-N- methylmethanesulfonamide | 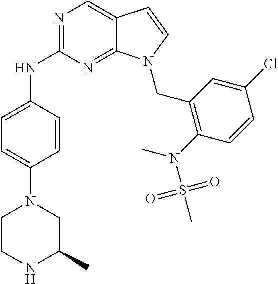 [SMILES: C[C@@H]1CN(c2ccc(Nc3ncc4ccn(Cc5cc(Cl)ccc5N(C)S(C)(=O)=O)c4n3)cc2)CCN1] | 541.18 |
| 900 | N-(4-chloro-2-((2-((4-(4-(2- hydroxyethyl)piperazin-1- yl)phenyl)amino)-7H- pyrrolo[2,3-d]pyrimidin-7- yl)methyl)phenyl)-N- methylmethanesulfonamide | 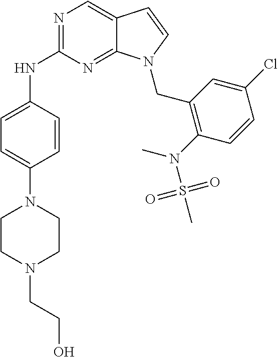 [SMILES: CN(c1ccc(Cl)cc1Cn1ccc2cnc(Nc3ccc(N4CCN(CCO)CC4)cc3)nc21)S(C)(=O)=O] | 571.19 |
| 901 | N-(4-chloro-2-((2-((4-(4- (dimethylamino)cyclohexyl) phenyl)amino)-7H-pyrrolo[2,3- d]pyrimidin-7- yl)methyl)phenyl)-N- methylmethanesulfonamide | 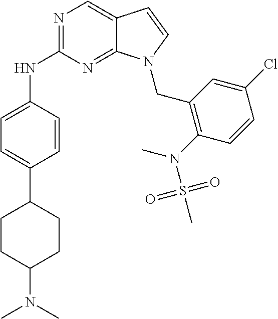 [SMILES: CN(C)C1CCC(c2ccc(Nc3ncc4ccn(Cc5cc(Cl)ccc5N(C)S(C)(=O)=O)c4n3)cc2)CC1] | 568.22 |
| 902 | (S)-N-(5-methoxy-3-((2-((4-(3- methylpiperazin-1- yl)phenyl)amino)-7H- pyrrolo[2,3-d]pyrimidin-7- yl)methyl)pyridin-2-yl)-N- methylmethanesulfonamide | 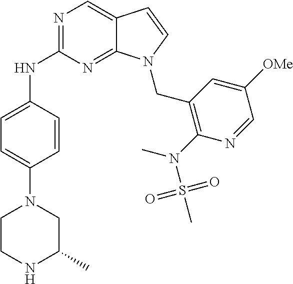 [SMILES: COc1cnc(N(C)S(C)(=O)=O)c(Cn2ccc3cnc(Nc4ccc(N5CCN[C@@H](C)C5)cc4)nc32)c1] | 537.24 |
| 903 | N-(4-((S)-3-methylpiperazin-1- yl)phenyl)-7-((2-(1- (methylsulfonyl)ethyl)pyridin- 3-yl)methyl)-7H-pyrrolo[2,3- d]pyrimidin-2-amine | 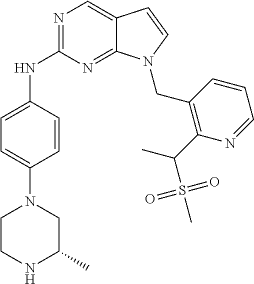 [SMILES: CC(c1ncccc1Cn1ccc2cnc(Nc3ccc(N4CCN[C@@H](C)C4)cc3)nc21)S(C)(=O)=O] | 506.23 |
| 904 | N-(4-((R)-3-methylpiperazin-1- yl)phenyl)-7-(2-(1- (methylsulfonyl)ethyl)benzyl)- 7H-pyrrolo[2,3-d]pyrimidin-2- amine | 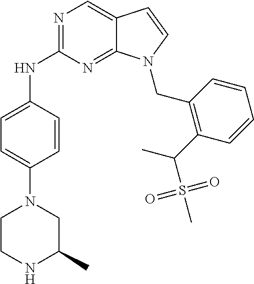 [SMILES: CC(c1ccccc1Cn1ccc2cnc(Nc3ccc(N4CCN[C@H](C)C4)cc3)nc21)S(C)(=O)=O] | 505.23 |
| 905 | (S)-N-(5-chloro-3-((2-((4-(3- methylpiperazin-1- yl)phenyl)amino)-7H- pyrrolo[2,3-d]pyrimidin-7- yl)methyl)pyridin-2-yl)-N- methylmethanesulfonamide | 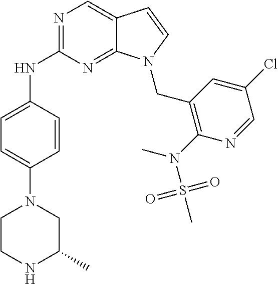 [SMILES: C[C@H]1CN(c2ccc(Nc3ncc4ccn(Cc5cc(Cl)cnc5N(C)S(C)(=O)=O)c4n3)cc2)CCN1] | 542.18 |
| 906 | 7-((2-(difluoromethoxy) pyridin-3-yl)methyl)-N- (4-(piperidin-4- yl)phenyl)-7H-pyrrolo[2,3- d]pyrimidin-2-amine | 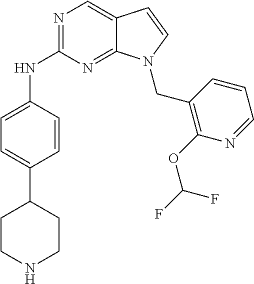 [SMILES: FC(F)Oc1ncccc1Cn1ccc2cnc(Nc3ccc(C4CCNCC4)cc3)nc21] | 451.19 |
| 907 | (5-((5,5-dimethyl-7-(pyridin-2- yl)-6,7-dihydro-5H- pyrrolo[2,3-d]pyrimidin-2- yl)amino)-1H-indol-3- yl)methanol | 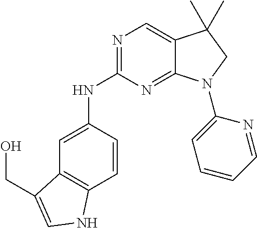 [SMILES: CC1(C)CN(c2ccccn2)c2nc(Nc3ccc4[nH]cc(CO)c4c3)ncc21] | 387.19 |
| 908 | N-(3-((dimethylamino)methyl)- 1H-indol-5-yl)-5,5-dimethyl-7- (pyridin-2-yl)-6,7-dihydro-5H- pyrrolo[2,3-d]pyrimidin-2- amine | 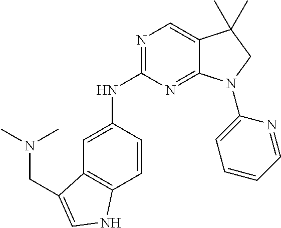 [SMILES: CN(C)Cc1c[nH]c2ccc(Nc3ncc4c(n3)N(c3ccccn3)CC4(C)C)cc12] | 414.23 |
| 909 | N-(3-isopropyl-1H-indol-5-yl)- 5,5-dimethyl-7-(pyridin-2-yl)- 6,7-dihydro-5H-pyrrolo[2,3- d]pyrimidin-2-amine | 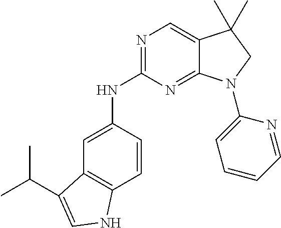 [SMILES: CC(C)c1c[nH]c2ccc(Nc3ncc4c(n3)N(c3ccccn3)CC4(C)C)cc12] | 399.22 |
| 910 | N5-(5,5-dimethyl-7-(pyridin-2- yl)-6,7-dihydro-5H- pyrrolo[2,3-d]pyrimidin-2- yl)-N3,N3-dimethyl-1H-indole- 3,5-diamine | 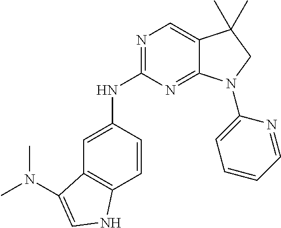 [SMILES: CN(C)c1c[nH]c2ccc(Nc3ncc4c(n3)N(c3ccccn3)CC4(C)C)cc12] | 400.22 |
| 911 | 5,5-dimethyl-N-(3-(prop-1-en- 2-yl)-1H-indol-5-yl)-7- (pyridin-2-yl)-6,7-dihydro-5H- pyrrolo[2,3-d]pyrimidin-2- amine | 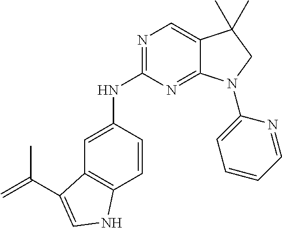 [SMILES: C=C(C)c1c[nH]c2ccc(Nc3ncc4c(n3)N(c3ccccn3)CC4(C)C)cc12] | 397.21 |
| 912 | N-methyl-N-(2-((2-((4- (piperazin-1-yl)phenyl)amino)- 7H-pyrrolo[2,3-d]pyrimidin-7- yl)methyl)cyclohexyl) methanesulfonamide | 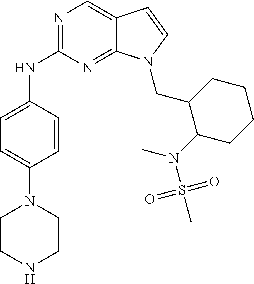 [SMILES: CN(C1CCCCC1Cn1ccc2cnc(Nc3ccc(N4CCNCC4)cc3)nc21)S(C)(=O)=O] | 498.26 |
| 913 | N-methyl-N-(1-((2-((4- (piperazin-1-yl)phenyl)amino)- 7H-pyrrolo[2,3-d]pyrimidin-7- yl)methyl)cyclohexyl) methanesulfonamide | 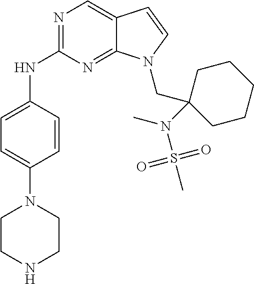 [SMILES: CN(C1(Cn2ccc3cnc(Nc4ccc(N5CCNCC5)cc4)nc32)CCCCC1)S(C)(=O)=O] | 498.26 |
| 914 | 4-(2-((4-(piperazin-1- yl)phenyl)amino)-7H- pyrrolo[2,3-d]pyrimidin-7-yl)- 3,4-dihydro-1H-pyrido[2,3- c][1,2]thiazine 2,2-dioxide | 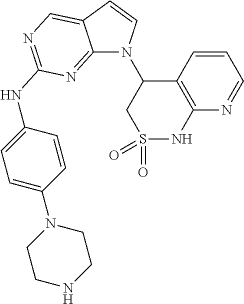 [SMILES: O=S1(=O)CC(n2ccc3cnc(Nc4ccc(N5CCNCC5)cc4)nc32)c2cccnc2N1] | 477.17 |
| 915 | 4-(2-((4-(piperazin-1- yl)phenyl)amino)-7H- pyrrolo[2,3-d]pyrimidin-7-yl)- 3,4-dihydro-1H-pyrido[2,3- c][1,2,6]thiadiazine 2,2-dioxide | 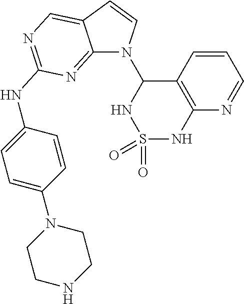 [SMILES: O=S1(=O)Nc2ncccc2C(n2ccc3cnc(Nc4ccc(N5CCNCC5)cc4)nc32)N1] | 478.19 |
| 916 | N-(4-(piperazin-1-yl)phenyl)-7- ((1,2,3,4-tetrahydroquinolin-8- yl)sulfonyl)-7H-pyrrolo[2,3- d]pyrimidin-2-amine | 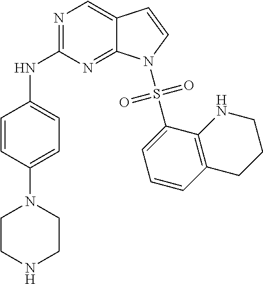 [SMILES: O=S(=O)(c1cccc2c1NCCC2)n1ccc2cnc(Nc3ccc(N4CCNCC4)cc3)nc21] | 490.19 |
| 917 | 8-((2-((4-(piperazin-1- yl)phenyl)amino)-7H- pyrrolo[2,3-d]pyrimidin-7- yl)sulfonyl)-2,3- dihydroquinolin-4(1H)-one | 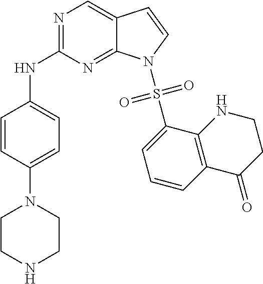 [SMILES: O=C1CCNc2c1cccc2S(=O)(=O)n1ccc2cnc(Nc3ccc(N4CCNCC4)cc3)nc21] | 504.17 |
| 918 | N-(4-(piperazin-1-yl)phenyl)-7- ((5,6,7,8-tetrahydroquinolin-8- yl)sulfonyl)-7H-pyrrolo[2,3- d]pyrimidin-2-amine | 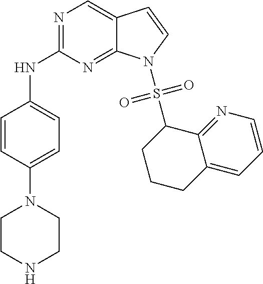 [SMILES: O=S(=O)(C1CCCc2cccnc21)n1ccc2cnc(Nc3ccc(N4CCNCC4)cc3)nc21] | 490.19 |
| 919 | (S)-N-(4-(dimethylamino)-2- ((2-((4-(3-methylpiperazin-1- yl)phenyl)amino)-7H- pyrrolo[2,3-d]pyrimidin-7- yl)methyl)phenyl)-N- methylmethanesulfonamide | 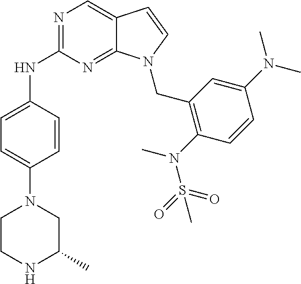 [SMILES: C[C@H]1CN(c2ccc(Nc3ncc4ccn(Cc5cc(N(C)C)ccc5N(C)S(C)(=O)=O)c4n3)cc2)CCN1] | 549.27 |
| 921 | N-(5-chloro-3-((2-((4-(4- (dimethylamino)piperidin-1- yl)phenyl)amino)-7H- pyrrolo[2,3-d]pyrimidin-7- yl)methyl)pyridin-2-yl)-N- methylmethanesulfonamide | 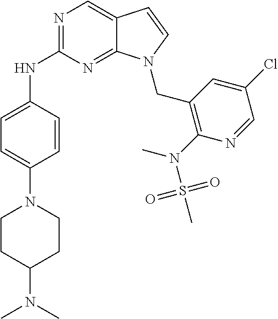 [SMILES: CN(C)C1CCN(c2ccc(Nc3ncc4ccn(Cc5cc(Cl)cnc5N(C)S(C)(=O)=O)c4n3)cc2)CC1] | 570.22 |
| 922 | N-(4-chloro-2-((2-((4-(1- methylpiperidin-4- yl)phenyl)amino)-7H- pyrrolo[2,3-c]pyrimidin-7- yl)methyl)phenyl)-N- methylmethanesulfonamide | 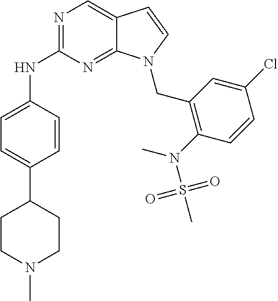 [SMILES: CN1CCC(c2ccc(Nc3ncc4ccn(Cc5cc(Cl)ccc5N(C)S(C)(=O)=O)c4n3)cc2)CC1] | 540.19 |
| 923 | N-(2-(2-(5,5-dimethyl-2-((4-(4- methylpiperazin-1- yl)phenyl)amino)-5,6-dihydro- 7H-pyrrolo[2,3-d]pyrimidin-7- yl)-2-oxoethyl)phenyl)-N- methylmethanesulfonamide | 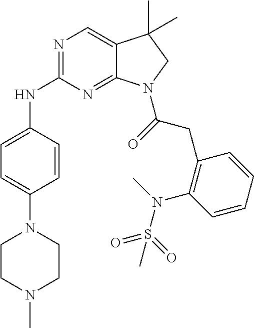 [SMILES: CN1CCN(c2ccc(Nc3ncc4c(n3)N(C(=O)Cc3ccccc3N(C)S(C)(=O)=O)CC4(C)C)cc2)CC1] | 564.27 |
| 924 | 5,5-difluoro-N-(4-(4- methylpiperazin-1-yl)phenyl)- 7-(quinolin-8-ylsulfonyl)-6,7- dihydro-5H-pyrrolo[2,3- d]pyrimidin-2-amine | 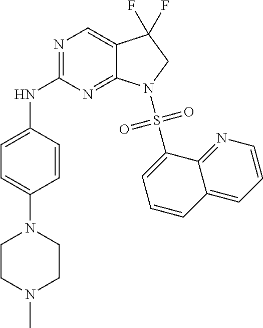 [SMILES: CN1CCN(c2ccc(Nc3ncc4c(n3)N(S(=O)(=O)c3cccc5cccnc35)CC4(F)F)cc2)CC1] | 538.18 |
| 925 | N-(5-chloro-3-((2-((4- (piperazin-1-yl)phenyl)amino)- 7H-pyrrolo[2,3-d]pyrimidin-7- yl)methyl)pyridin-2-yl)-N- methylmethanesulfonamide | 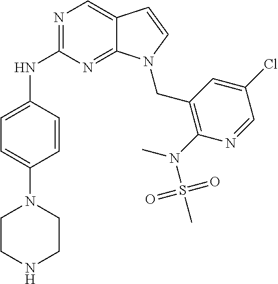 [SMILES: CN(c1ncc(Cl)cc1Cn1ccc2cnc(Nc3ccc(N4CCNCC4)cc3)nc21)S(C)(=O)=O] | 528.16 |
| 926 | (S)-N-(5-chloro-3-((2-((4-(3- methylpiperazin-1- yl)phenyl)amino)-7H- pyrrolo[2,3-d]pyrimidin-7- yl)methyl)pyridin-2-yl)-N- methylmethanesulfonamide | 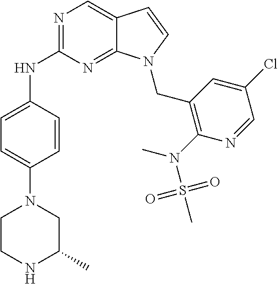 [SMILES: C[C@H]1CN(c2ccc(Nc3ncc4ccn(Cc5cc(Cl)cnc5N(C)S(C)(=O)=O)c4n3)cc2)CCN1] | 542.18 |
| 927 | (S)-N-(5-(dimethylamino)-3- ((2-((4-(3-methylpiperazin-1- yl)phenyl)amino)-7H- pyrrolo[2,3-d]pyrimidin-7- yl)methyl)pyridin-2-yl)-N- methylmethanesulfonamide | 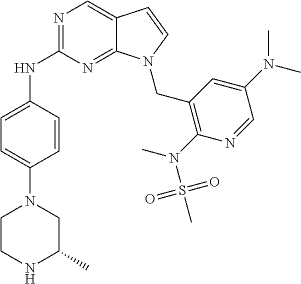 [SMILES: C[C@H]1CN(c2ccc(Nc3ncc4ccn(Cc5cc(N(C)C)cnc5N(C)S(C)(=O)=O)c4n3)cc2)CCN1] | 550.27 |
| 928 | N-(4-(dimethylamino)-2-((2- ((4-(4-(2- hydroxyethyl)piperazin-1- yl)phenyl)amino)-7H- pyrrolo[2,3-d]pyrimidin-7- yl)methyl)phenyl)-N- methylmethanesulfonamide | 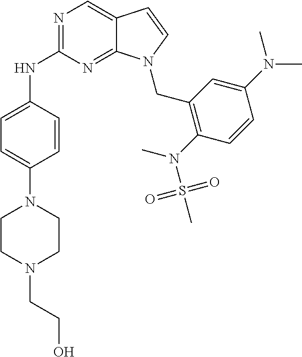 [SMILES: CN(C)c1ccc(N(C)S(C)(=O)=O)c(Cn2ccc3cnc(Nc4ccc(N5CCN(CCO)CC5)cc4)nc32)c1] | 579.28 |
| 929 | N-(5-fluoro-3-((2-((4- (piperazin-1-yl)phenyl)amino)- 7H-pyrrolo[2,3-d]pyrimidin-7- yl)methyl)pyridin-2-yl)-N- methylmethanesulfonamide | 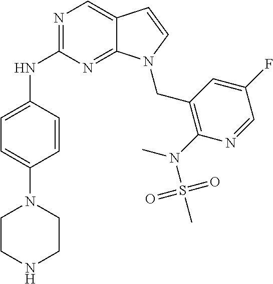 [SMILES: CN(c1ncc(F)cc1Cn1ccc2cnc(Nc3ccc(N4CCNCC4)cc3)nc21)S(C)(=O)=O] | 511.2 |
| 930 | N-methyl-N-(3-((2-((4- (piperazin-1-yl)phenyl)amino)- 7H-pyrrolo[2,3-d]pyrimidin-7- yl)methyl)-5- (trifluoromethyl)pyridin-2- yl)methanesulfonamide | 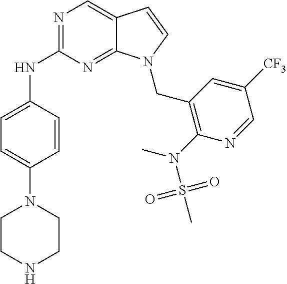 [SMILES: CN(c1ncc(C(F)(F)F)cc1Cn1ccc2cnc(Nc3ccc(N4CCNCC4)cc3)nc21)S(C)(=O)=O] | 561.2 |
| 931 | (S)-N-methyl-N-(3-((2-((4-(3- methylpiperazin-1- yl)phenyl)amino)-7H- pyrrolo[2,3-d]pyrimidin-7- yl)methyl)-5- (trifluoromethyl)pyridin-2- yl)methanesulfonamide | 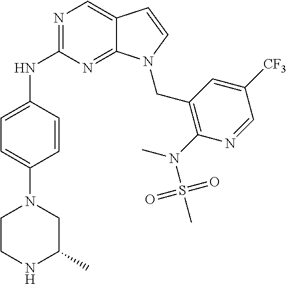 [SMILES: C[C@H]1CN(c2ccc(Nc3ncc4ccn(Cc5cc(C(F)(F)F)cnc5N(C)S(C)(=O)=O)c4n3)cc2)CCN1] | 575.21 |
| 932 | (R)-N-(5-fluoro-3-((2-((4-(3- methylpiperazin-1- yl)phenyl)amino)-7H- pyrrolo[2,3-d]pyrimidin-7- yl)methyl)pyridin-2-yl)-N- methylmethanesulfonamide | 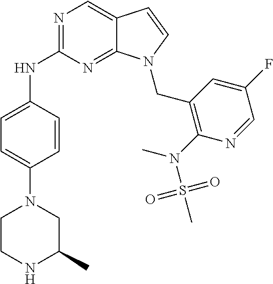 [SMILES: C[C@@H]1CN(c2ccc(Nc3ncc4ccn(Cc5cc(F)cnc5N(C)S(C)(=O)=O)c4n3)cc2)CCN1] | 525.22 |
| 933 | (R)-N-methyl-N-(3-((2-((4-(3- methylpiperazin-1- yl)phenyl)amino)-7H- pyrrolo[2,3-d]pyrimidin-7- yl)methyl)-5- (trifluoromethyl)pyridin-2- yl)methanesulfonamide | 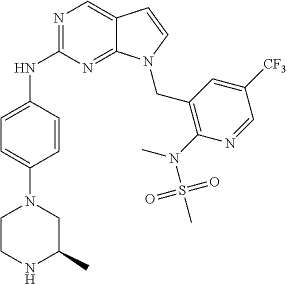 [SMILES: C[C@@H]1CN(c2ccc(Nc3ncc4ccn(Cc5cc(C(F)(F)F)cnc5N(C)S(C)(=O)=O)c4n3)cc2)CCN1] | 575.21 |
| 934 | N-(4-(dimethylamino)-2-((2- ((4-(piperidin-4- yl)phenyl)amino)-7H- pyrrolo[2,3-d]pyrimidin-7- yl)methyl)phenyl)-N- methylmethanesulfonamide | 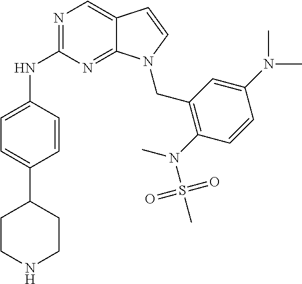 [SMILES: CN(C)c1ccc(N(C)S(C)(=O)=O)c(Cn2ccc3cnc(Nc4ccc(C5CCNCC5)cc4)nc32)c1] | 534.26 |
| 935 | N-(4-fluoro-2-((2-((4-(1- methylpiperidin-4- yl)phenyl)amino)-7H- pyrrolo[2,3-d]pyrimidin-7- yl)methyl)phenyl)-N- methylmethanesulfonamide | 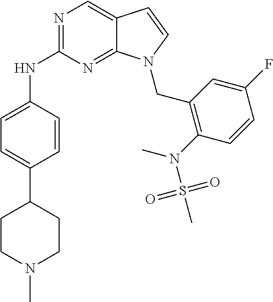 [SMILES: CN1CCC(c2ccc(Nc3ncc4ccn(Cc5cc(F)ccc5N(C)S(C)(=O)=O)c4n3)cc2)CC1] | 523.22 |
| 936 | N-methyl-N-(2-methyl-6-((2- ((4-(piperazin-1- yl)phenyl)amino)-7H- pyrrolo[2,3-d]pyrimidin-7- yl)methyl)phenyl) methanesulfonamide | 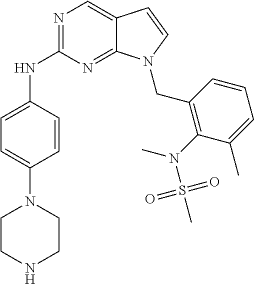 [SMILES: Cc1cccc(Cn2ccc3cnc(Nc4ccc(N5CCNCC5)cc4)nc32)c1N(C)S(C)(=O)=O] | 506.23 |
| 937 | N-(2-chloro-6-((2-((4- (piperazin-1-yl)phenyl)amino)- 7H-pyrrolo[2,3-d]pyrimidin-7- yl)methyl)phenyl)-N- methylmethanesulfonamide | 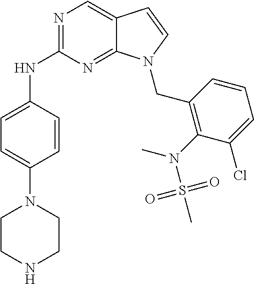 [SMILES: CN(c1c(Cl)cccc1Cn1ccc2cnc(Nc3ccc(N4CCNCC4)cc3)nc21)S(C)(=O)=O] | 527.17 |
| 938 | N-(2-cyano-6-((2-((4- (piperazin-1-yl)phenyl)amino)- 7H-pyrrolo[2,3-d]pyrimidin-7- yl)methyl)phenyl)-N- methylmethanesulfonamide | 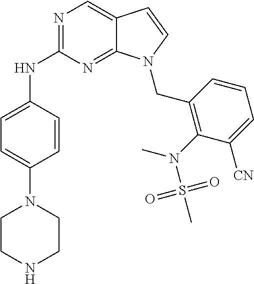 [SMILES: CN(c1c(C#N)cccc1Cn1ccc2cnc(Nc3ccc(N4CCNCC4)cc3)nc21)S(C)(=O)=O] | 517.17 |
| 939 | N-(2-fluoro-6-((2-((4- (piperazin-1-yl)phenyl)amino)- 7H-pyrrolo[2,3-d]pyrimidin-7- yl)methyl)phenyl)-N- methylmethanesulfonamide | 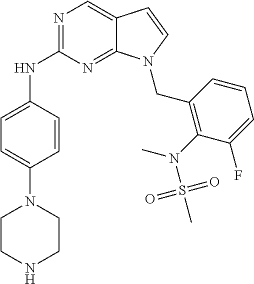 [SMILES: CN(c1c(F)cccc1Cn1ccc2cnc(Nc3ccc(N4CCNCC4)cc3)nc21)S(C)(=O)=O] | 510.2 |
| 940 | N-(2-(dimethylamino)-6-((2- ((4-(piperazin-1- yl)phenyl)amino)-7H- pyrrolo[2,3-d]pyrimidin-7- yl)methyl)phenyl)-N- methylmethanesulfonamide | 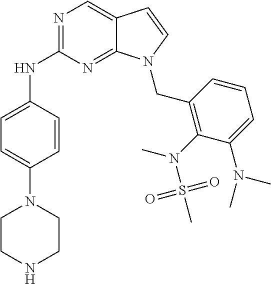 [SMILES: CN(C)c1cccc(Cn2ccc3cnc(Nc4ccc(N5CCNCC5)cc4)nc32)c1N(C)S(C)(=O)=O] | 535.26 |
| 943 | N-(2-((2-((4-(4- hydroxypiperidin-1- yl)phenyl)amino)-7H- pyrrolo[2,3-d]pyrimidin-7- yl)methyl)-4-methylphenyl)-N- methylmethanesulfonamide | 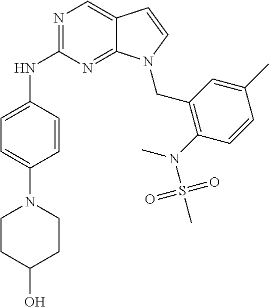 [SMILES: Cc1ccc(N(C)S(C)(=O)=O)c(Cn2ccc3cnc(Nc4ccc(N5CCC(O)CC5)cc4)nc32)c1] | 521.22 |
| 944 | N-(2-((2-((4-(4- (dimethylamino)piperidin-1- yl)-3-methoxyphenyl)amino)- 7H-pyrrolo[2,3-d]pyrimidin-7- yl)methyl)-4-methylphenyl)-N- methylmethanesulfonamide | 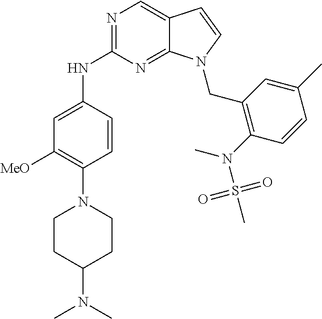 [SMILES: COc1cc(Nc2ncc3ccn(Cc4cc(C)ccc4N(C)S(C)(=O)=O)c3n2)ccc1N1CCC(N(C)C)CC1] | 578.29 |
| 945 | N-(2-((2-((4-(4- (dimethylamino)piperidin-1- yl)-3-fluorophenyl)amino)-7H- pyrrolo[2,3-d]pyrimidin-7- yl)methyl)-4-methylphenyl)-N- methylmethanesulfonamide | 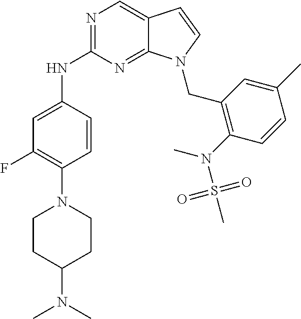 [SMILES: Cc1ccc(N(C)S(C)(=O)=O)c(Cn2ccc3cnc(Nc4ccc(N5CCC(N(C)C)CC5)c(F)c4)nc32)c1] | 566.26 |
| 946 | N-(3-((2-((3-fluoro-4- (piperazin-1-yl)phenyl)amino)- 7H-pyrrolo[2,3-d]pyrimidin-7- yl)methyl)-5-methoxypyridin- 2-yl)-N- methylmethanesulfonamide | 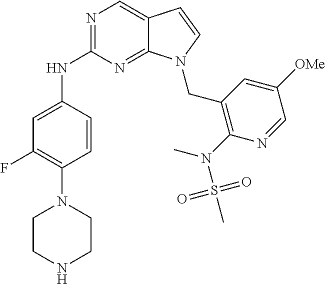 [SMILES: COc1cnc(N(C)S(C)(=O)=O)c(Cn2ccc3cnc(Nc4ccc(N5CCNCC5)c(F)c4)nc32)c1] | 541.21 |
| 947 | N-(2-((2-((4-(4- (dimethylamino)piperidin-1- yl)-3-fluorophenyl)amino)-7H- pyrrolo[2,3-d]pyrimidin-7- yl)methyl)-4-methoxyphenyl)- N-methylmethanesulfonamide | 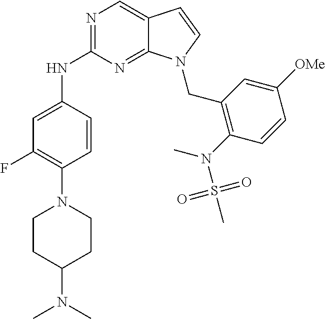 [SMILES: COc1ccc(N(C)S(C)(=O)=O)c(Cn2ccc3cnc(Nc4ccc(N5CCC(N(C)C)CC5)c(F)c4)nc32)c1] | 582.26 |
| 948 | N-(2-((2-((4-(4- hydroxypiperidin-1- yl)phenyl)amino)-7H- pyrrolo[2,3-d]pyrimidin-7- yl)methyl)-4-methoxyphenyl)- N-methylmethanesulfonamide | 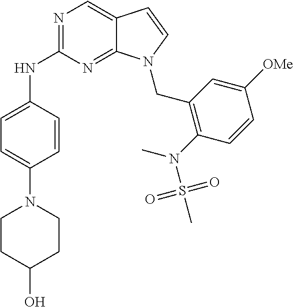 [SMILES: COc1ccc(N(C)S(C)(=O)=O)c(Cn2ccc3cnc(Nc4ccc(N5CCC(O)CC5)cc4)nc32)c1] | 535.26 |
| 949 | N-(5-fluoro-3-((2-((3-methoxy- 4-(piperazin-1- yl)phenyl)amino)-7H- pyrrolo[2,3-d]pyrimidin-7- yl)methyl)pyridin-2-yl)-N- methylmethanesulfonamide | 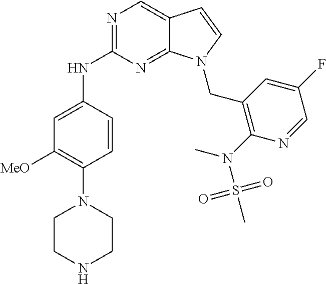 [SMILES: COc1cc(Nc2ncc3ccn(Cc4cc(F)cnc4N(C)S(C)(=O)=O)c3n2)ccc1N1CCNCC1] | 541.21 |
| 950 | N-(4-fluoro-2-((2-((4-(4- hydroxypiperidin-1- yl)phenyl)amino)-7H- pyrrolo[2,3-d]pyrimidin-7- yl)methyl)phenyl)-N- methylmethanesulfonamide | 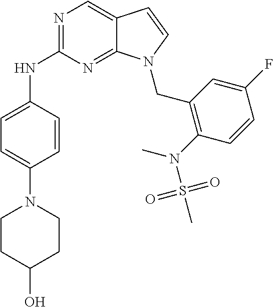 [SMILES: CN(c1ccc(F)cc1Cn1ccc2cnc(Nc3ccc(N4CCC(O)CC4)cc3)nc21)S(C)(=O)=O] | 525.26 |
| 952 | N-methyl-N-(3-((2-((4- (piperazin-1-yl)phenyl)amino)- 7H-pyrrolo[2,3-d]pyrimidin-7- yl)methyl)pyrazin-2- yl)methanesulfonamide | 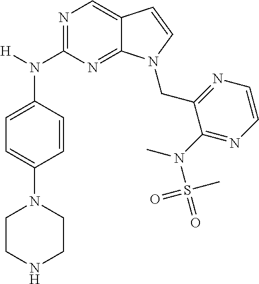 [SMILES: CN(c1nccnc1Cn1ccc2cnc(Nc3ccc(N4CCNCC4)cc3)nc21)S(C)(=O)=O] | 494.20 |
| 954 | N-methyl-N-(3-((2-((4- (piperazine-1- carbonyl)phenyl)amino)-7H- pyrrolo[2,3-d]pyrimidin-7- yl)methyl)pyrazin-2- yl)methanesulfonamide | 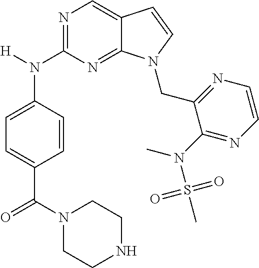 [SMILES: CN(c1nccnc1Cn1ccc2cnc(Nc3ccc(C(=O)N4CCNCC4)cc3)nc21)S(C)(=O)=O] | 522.2 |
| 955 | N-(3-((2-((4-(4-(2- hydroxyethyl)piperazin-1- yl)phenyl)amino)-7H- pyrrolo[2,3-d]pyrimidin-7- yl)methyl)pyrazin-2-yl)-N- methylmethanesulfonamide | 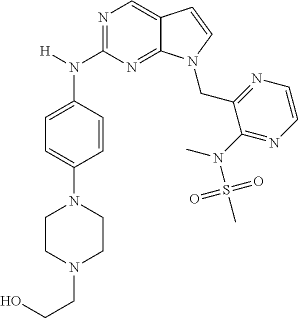 [SMILES: CN(c1nccnc1Cn1ccc2cnc(Nc3ccc(N4CCN(CCO)CC4)cc3)nc21)S(C)(=O)=O] | 538.23 |
| 956 | N-(3-((2-((4-(4- hydroxypiperidin-1- yl)phenyl)amino)-7H- pyrrolo[2,3-d]pyrimidin-7- yl)methyl)pyrazin-2-yl)-N- methylmethanesulfonamide | 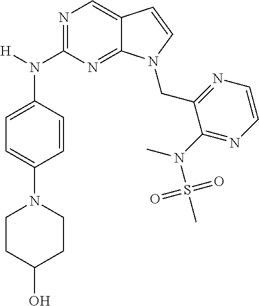 [SMILES: CN(c1nccnc1Cn1ccc2cnc(Nc3ccc(N4CCC(O)CC4)cc3)nc21)S(C)(=O)=O] | 509.2 |
| 957 | (R)-N-methyl-N-(3-((2-((4-(3- methylpiperazin-1- yl)phenyl)amino)-7H- pyrrolo[2,3-d]pyrimidin-7- yl)methyl)pyrazin-2- yl)methanesulfonamide | 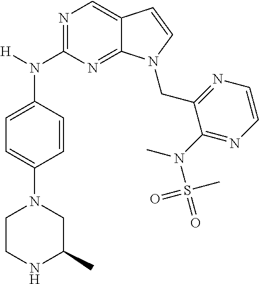 [SMILES: C[C@@H]1CN(c2ccc(Nc3ncc4ccn(Cc5nccnc5N(C)S(C)(=O)=O)c4n3)cc2)CCN1] | 508.22 |
| 959 | N-methyl-4-((1-((3-(N- methylmethylsulfonamido) pyrazin-2-yl)methyl)-1H- pyrrolo[3,2-c]pyridin-6- yl)amino)benzamide | 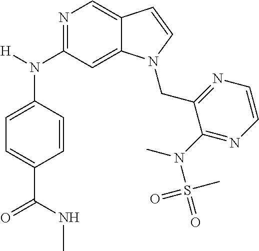 [SMILES: CNC(=O)c1ccc(Nc2cc3c(ccn3Cc3nccnc3N(C)S(C)(=O)=O)cn2)cc1] | 466.16 |
| 960 | N-methyl-N-(3-((6-((4- (piperazin-1-yl)phenyl)amino)- 1H-pyrrolo[3,2-c]pyridin-1- yl)methyl)pyrazin-2- yl)methanesulfonamide | 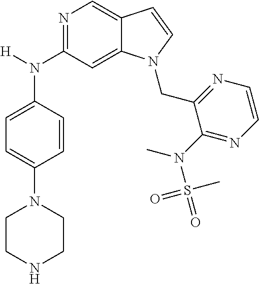 [SMILES: CN(c1nccnc1Cn1ccc2cnc(Nc3ccc(N4CCNCC4)cc3)cc21)S(C)(=O)=O] | 493.21 |
| 961 | (S)-N-methyl-N-(3-((6-((4-(3- methylpiperazin-1- yl)phenyl)amino)-1H- pyrrolo[3,2-c]pyridin-1- yl)methyl)pyrazin-2- yl)methanesulfonamide | 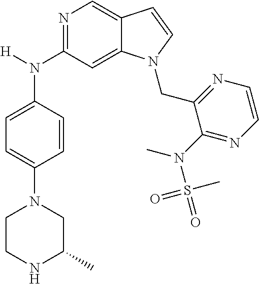 [SMILES: C[C@H]1CN(c2ccc(Nc3cc4c(ccn4Cc4nccnc4N(C)S(C)(=O)=O)cn3)cc2)CCN1] | 507.22 |
| 962 | N-methyl-4-((1-((3-(N- methylmethylsulfonamido) pyrazin-2-yl)methyl)-1H- pyrazolo[4,3-c]pyridin-6- yl)amino)benzamide | 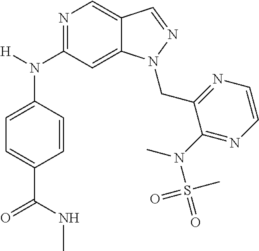 [SMILES: CNC(=O)c1ccc(Nc2cc3c(cn2)cnn3Cc2nccnc2N(C)S(C)(=O)=O)cc1] | 467.16 |
| 963 | N-methyl-N-(3-((6-((4- (piperazin-1-yl)phenyl)amino)- 1H-pyrazolo[4,3-c]pyridin-1- yl)methyl)pyrazin-2- yl)methanesulfonamide | 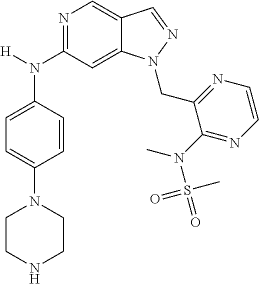 [SMILES: CN(c1nccnc1Cn1ncc2cnc(Nc3ccc(N4CCNCC4)cc3)cc21)S(C)(=O)=O] | 494.2 |
| 964 | (S)-N-methyl-N-(3-((6-((4-(3- methylpiperazin-1- yl)phenyl)amino)-1H- pyrazolo[4,3-c]pyridin-1- yl)methyl)pyrazin-2- yl)methanesulfonamide | 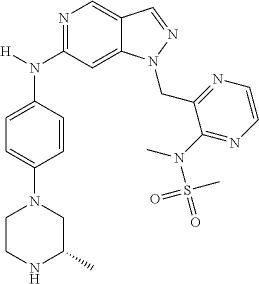 [SMILES: C[C@H]1CN(c2ccc(Nc3cc4c(cn3)cnn4Cc3nccnc3N(C)S(C)(=O)=O)cc2)CCN1] | 508.22 |
| 965 | N-methyl-4-((1-((3-(N- methylmethylsulfonamido) pyrazin-2-yl)methyl)-1H- pyrazolo[3,4-d]pyrimidin-6- yl)amino)benzamide | 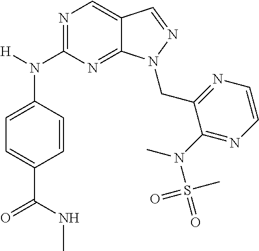 [SMILES: CNC(=O)c1ccc(Nc2ncc3cnn(Cc4nccnc4N(C)S(C)(=O)=O)c3n2)cc1] | 468.15 |
| 966 | N-methyl-N-(3-((6-((4- (piperazin-1-yl)phenyl)amino)- 1H-pyrazolo[3,4-d]pyrimidin- 1-yl)methyl)pyrazin-2- yl)methanesulfonamide | 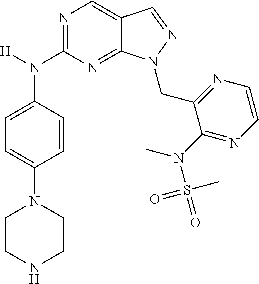 [SMILES: CN(c1nccnc1Cn1ncc2cnc(Nc3ccc(N4CCNCC4)cc3)nc21)S(C)(=O)=O] | 495.2 |
| 967 | (S)-N-methyl-N-(3-((6-((4-(3- methylpiperazin-1- yl)phenyl)amino)-1H- pyrazolo[3,4-d]pyrimidin-1- yl)methyl)pyrazin-2- yl)methanesulfonamide | 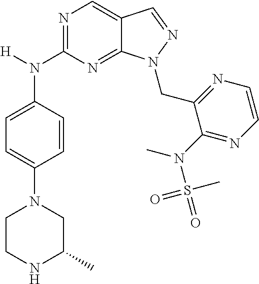 [SMILES: C[C@H]1CN(c2ccc(Nc3ncc4cnn(Cc5nccnc5N(C)S(C)(=O)=O)c4n3)cc2)CCN1] | 509.22 |
| 968 | N-(5-fluoro-3-((2-((4- (piperazin-1-yl)phenyl)amino)- 7H-pyrrolo[2,3-d]pyrimidin-7- yl)methyl)pyrazin-2-yl)-N- methylmethanesulfonamide | 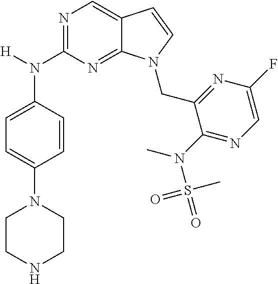 [SMILES: CN(c1ncc(F)nc1Cn1ccc2cnc(Nc3ccc(N4CCNCC4)cc3)nc21)S(C)(=O)=O] | 512.19 |
| 969 | N-(6-fluoro-3-((2-((4- (piperazin-1-yl)phenyl)amino)- 7H-pyrrolo[2,3-d]pyrimidin-7- yl)methyl)pyrazin-2-yl)-N- methylmethanesulfonamide | 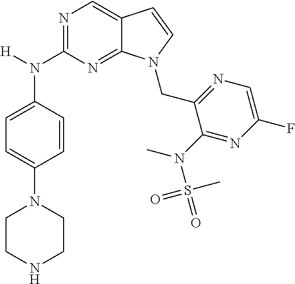 [SMILES: CN(c1nc(F)cnc1Cn1ccc2cnc(Nc3ccc(N4CCNCC4)cc3)nc21)S(C)(=O)=O] | 512.19 |
| 970 | N-methyl-N-(5-methyl-3-((2- ((4-(piperazin-1- yl)phenyl)amino)-7H- pyrrolo[2,3-d]pyrimidin-7- yl)methyl)pyrazin-2- yl)methanesulfonamide | 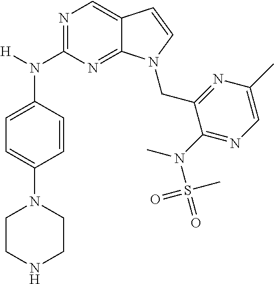 [SMILES: Cc1cnc(N(C)S(C)(=O)=O)c(Cn2ccc3cnc(Nc4ccc(N5CCNCC5)cc4)nc32)n1] | 508.22 |
| 971 | N-methyl-N-(6-methyl-3-((2- ((4-(piperazin-1- yl)phenyl)amino)-7H- pyrrolo[2,3-d]pyrimidin-7- yl)methyl)pyrazin-2- yl)methanesulfonamide | 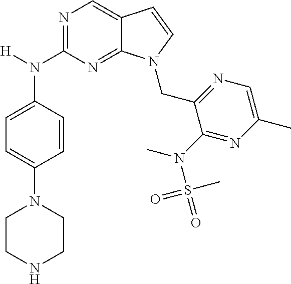 [SMILES: Cc1cnc(Cn2ccc3cnc(Nc4ccc(N5CCNCC5)cc4)nc32)c(N(C)S(C)(=O)=O)n1] | 508.22 |
| 972 | N-(5-methoxy-3-((2-((4- (piperazin-1-yl)phenyl)amino)- 7H-pyrrolo[2,3-d]pyrimidin-7- yl)methyl)pyrazin-2-yl)-N- methylmethanesulfonamide | 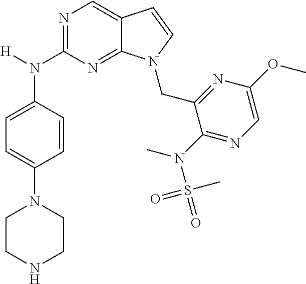 [SMILES: COc1cnc(N(C)S(C)(=O)=O)c(Cn2ccc3cnc(Nc4ccc(N5CCNCC5)cc4)nc32)n1] | 524.21 |
| 973 | N-(6-methoxy-3-((2-((4- (piperazin-1-yl)phenyl)amino)- 7H-pyrrolo[2,3-d]pyrimidin-7- yl)methyl)pyrazin-2-yl)-N- methylmethanesulfonamide | 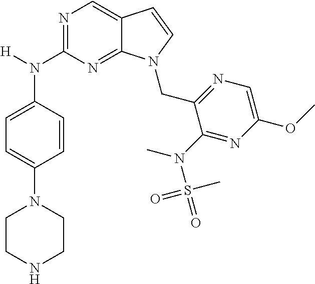 [SMILES: COc1cnc(Cn2ccc3cnc(Nc4ccc(N5CCNCC5)cc4)nc32)c(N(C)S(C)(=O)=O)n1] | 524.21 |
| 974 | N-(5-chloro-3-((2-((4- (piperazin-1-yl)phenyl)amino)- 7H-pyrrolo[2,3-d]pyrimidin-7- yl)methyl)pyrazin-2-yl)-N- methylmethanesulfonamide | 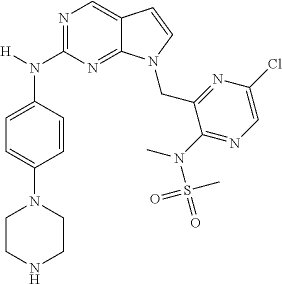 [SMILES: CN(c1ncc(Cl)nc1Cn1ccc2cnc(Nc3ccc(N4CCNCC4)cc3)nc21)S(C)(=O)=O] | 529.16 |
| 975 | N-(6-chloro-3-((2-((4- (piperazin-1-yl)phenyl)amino)- 7H-pyrrolo[2,3-d]pyrimidin-7- yl)methyl)pyrazin-2-yl)-N- methylmethanesulfonamide | 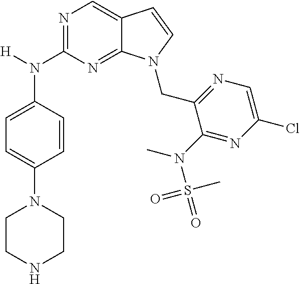 [SMILES: CN(c1nc(Cl)cnc1Cn1ccc2cnc(Nc3ccc(N4CCNCC4)cc3)nc21)S(C)(=O)=O] | 529.16 |

### Example 976

### Biology Assay Protocols

### A. FAK1 HTRF Assay

### (i) Reagents

Enzyme Assay Buffer

60 mM HEPES (pH 7.4)
50 mM NaCl
20 mM MgCl₂
5 mM MnCl₂
filtered using 0.2 μm pore size and store at 4° C.

Supplements (added fresh to the enzyme buffer)
Make 1M DTT stock, 5% BSA stock and 0.1M Na₃OV₄ stored at 4° C.

| | | |
|---|---|---|
| Above buffer | 100.00 | mL |
| 1M DTT | 200 | μL |
| 5% BSA | 1000 | μL |
| 0.1M Na₃OV₄ | 100 | μL |

### His's-hFAK1

Active FAK1 was prepared for use in the Homogeneous Time-Resolved Fluorescence (HTRF) assay. Briefly, N-terminal 6 His tagged recombinant human FAK1, from residue 692-end (GenBank accession no. L13616) was expressed by Bacculovirus in sf9 cells. The resulting protein had a molecular weight of 35.5 kDa. A 1.6 mg/mL (45 μM) stock was used in the HTRF assay at a final concentration of 10 nM.

| hFAK 1 working solution | RT | |
|---|---|---|
| | 1 plate (8x-8 nM) | |
| Enzyme buffer | 998.3 | μL |
| Fak 1 | 1.7 | μL |

### Gastrin Precursor-Biotinylated

Gastrin precursor (tyr87) biotinylated peptide which contains the residues surrounding tyrosine 87 (EEAY*GWM) of gastrin precursor was obtained (Cell Signaling Tech, Item no. 1310, Lot 7). The peptide was provided at a stock concentration of 6 μM, and used a final concentration of 0.5 μM in the HTRF assay.

### ATP

10 mM stock in 100 μL Enzyme assay buffer pH7.4 was prepared freshly and used at a final concentration of 10 μM in the assay.

| Gastrin/ATP working solution | RT (4x) | |
|---|---|---|
| (2 μM Gastrin, 40 μM ATP) | 1 | plate |
| Enzyme buffer | 1325 | μL |
| 10 mM ATP | 8 | μL |
| Gastrin (6 μM) | 667 | μL |

### Test Compounds

Powders were dissolved in 100% DMSO to a final stock concentration of 10 mM. The volume needed (in μL) to add for 10 mM=mg powder is 10⁵/MW of compound. The final compound concentration in the assay plate started at 10 μM.

### Reference:

Pfizer FAK inhibitor PF-562271 (N-methyl-N-(3-(((2-((2-oxoindolin-5-yl)amino)-5-(trifluoromethyl)pyrimidin-4-yl)amino)methyl)pyridin-2-yl)methanesulfonamide) was prepared as per the reported procedure from US Patent Publication No. 2005/256145A1.

HTRF buffer

50 mM Tris-HCl, pH-7.5
100 mM NaCl
0.1% BSA
0.05% Tween20 ™
0.5 mM EDTA
filter using 0.2 μm pore size, stored at 4° C.

### Phycolink® Streptavidin-Allophycocyanin (SA-APC)

Streptavidin-Allophycocyanin was obtained (Prozyme, Cat. No. PJ25S. SA-APC was provided at a stock concentration of 2.06 mg/mL, where APC concentration was 11.6 μM and the streptavidin concentration was 15.5 μM. SA-APC was used at a final concentration of 12 nM in the assay (based on streptavidin concentration).

### Europium-W1024-PT-66 Anti Phospho Tyrosine Ab

This reagent was obtained from Perkin ElmerAD0068. Europium-W1024-PT-66 Anti Phospho Tyrosine Ab is a monoclonal IgG1 that recognizes phospho tyrosine peptide. 100 μg/mL stock was used at a final concentration of 0.1 nM in the assay.

HTRF mix

| | |
|---|---|
| 2 plate | 4plate |

| | | | | |
|---|---|---|---|---|
| HTRF buffer | 25 | mL | 50 | mL |
| SA-APC | 20 | μL | 40 | μL |
| Eu-PT66Ab | 1 | μL | 2 | μL |

The above mix was prepared at least half hour before use.

### (ii) Assay Procedure

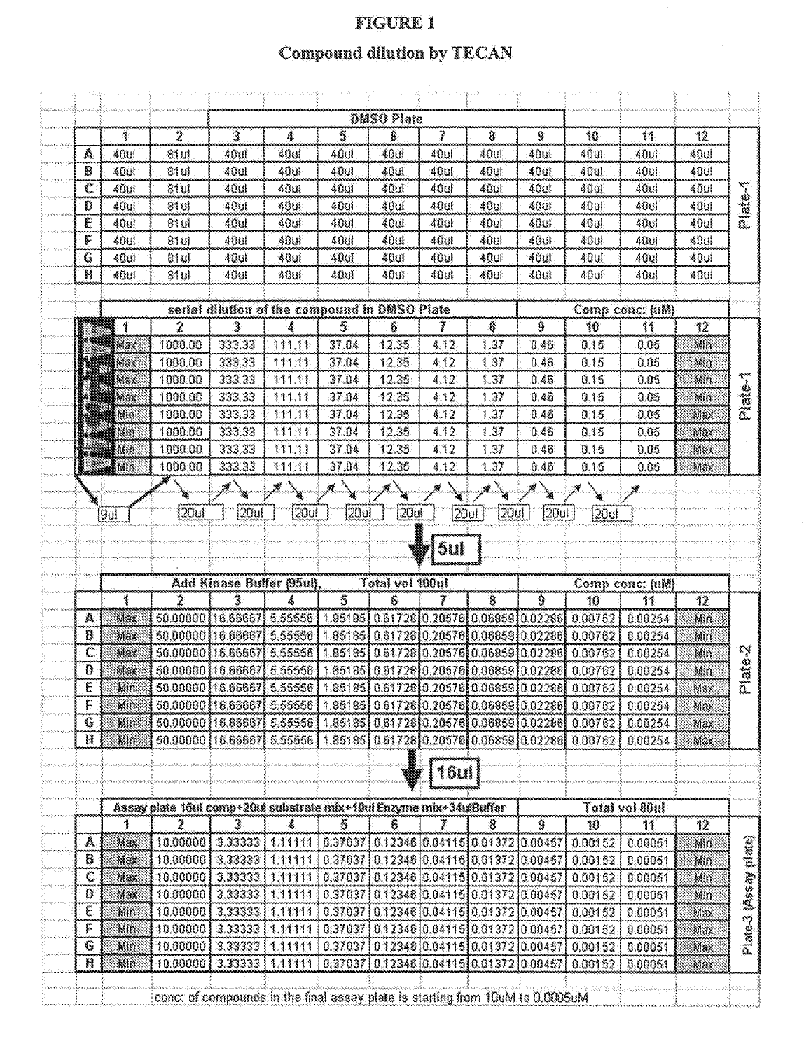

1. The compound dilutions were made by TECAN liquid handler as per the scheme in FIG. 1.
2. 16 μL of diluted compound was added to each well of the assay plate using TECAN liquid handler.
3. 20 μL of substrate mix (biotinylated gastrin precursor) was added into each well of the assay plate manually.
4. 10 μL of enzyme mix (FAK1) was added to each well except mins.
5. The plate was incubated at rt for 45 min with shaking.
6. The HTRF mix was prepared and 75 μL was added to the HTRF plate.
7. After the incubation was over, 10 μL of the mixture was transferred to the HTRF assay plate and incubated for 45 min at rt with shaking.
8. The readings were taken in Pherastar in HTRF mode (ext 337 nm, em 665 & 620 nm)
9. The results were analyzed using Graphpad Prism® software after calculation the z factor signal window and % inhibition.
10. The IC₅₀ was calculated.

### B. Src HTRF ASSAY

### (i) Reagents

Enzyme assay buffer, supplements, and compounds were prepared as described in the FAK HTRF assay.

### Src, Active

Recombinant Src enzyme expressed in Sf21 insect cells was obtained (Millipore, Cat No. 14-326, Lot # D8CN006U-K). A 35 μM stock was used at a final concentration of 5 nM in the assay.

| hSrc 1 working solution | RT | |
|---|---|---|
| | 1 plate (8x-40 nM) | |
| Enzyme buffer | 998.7 | μL |
| Src | 1.3 | μL |

Tyk2 synthetic peptide-Biotinylated-N-terminal tag-EQEDEPEGDYFEWLE

A custom made substrate, a Tyk2 synthetic peptide, biotinylated with an N-terminal tag was obtained (Biopeptide and Sigma, Cat. No. 14918001 VC80021). A 100 μM stock was used at a final concentration of 0.5 μM in the assay.

### ATP

A 10 mM stock in 100 μL enzyme assay buffer pH7.4 was prepared freshly and used at a final concentration of 10 μM in the assay.

| Gastrin/ATP working solution | RT (4x) | |
|---|---|---|
| (2 μM Gastrin, 40 μM ATP) | 1 | plate |
| Enzyme buffer | 1972 | μL |
| 10 mM ATP | 8 | μL |
| Tyk2 substrate | 40 | μL |

### Reference:

Dasatinib (Bristol-Myers Squibb)

The assay procedure was the same as discussed above. See FIG. 2 for plate set up.

### C. Aurora B HTRF Assay

### (i) Reagents:

Enzyme Assay Buffer

50 mM HEPES (pH 7.4)
1 mM EGTA
10 mM MgCl₂
2 mM DTT
0.01% Tween20 ™
Prepare 40 mL/plate, use milliQ ® water fresh every time.

Aurora B Enzyme

AURKB (Aurora B) enzyme was obtained (Invitrogen, Cat No. PVR 130, Lot No. 857013J). A 8.64 μM stock was used at a final concentration 5 nM in the assay.

| Aurora B working solution | (4x-20 nM) | |
|---|---|---|
| | 1 | plate |
| Enzyme | 1.1 | μL |
| Buffer | 500 | μL |

Lance@Ultera Ulight-PLK (Ser137) peptide substrate

Peptide substrate was obtained from Perkin Elmer (Lot#1629793, TRF0110-M). A 5 μM stock was used at a final concentration of 50 nM in the assay.

ATP

10 mM stock prepared freshly as above, used at a final concentration of 30 μM in assay.

| substrate/ATP working solution | RT (2X) | |
|---|---|---|
| (5 μM Ulight-PLK(Ser137), 30 μM ATP) | 1 | plate |
| Enzyme buffer | 1168 | μL |
| Substrate | 24 | μL |
| ATP | 7.2 | μL |

The plate was covered with aluminum foil, put on a shaker and incubated 1 h at rt.

Reference: Pfizer aurora inhibitor PF-03814735 was procured from Selleckchem: Cat# S2725

### (ii) Assay Procedure

Using stock Lance detection buffer, 1× Lance detection buffer was prepared.
1. 10× lance detection buffer was diluted to 1×, mixed well and 100 μL was removed, and was used to substitute with antibody and EDTA (for a total of 2200 μL).
2. 88 μL of 10 mM EDTA was added from 500 mM stock.
3. 14 μL Europium labeled antibody (LANCE@ Ultra EU-Anti-P-PLK(Ser 137), TRF0203-D, Lot No. 1630351, Perkin Elmer) was added from 625 nM stock; required concentration was 2 nM in the assay.
4. The buffer well was mixed and 20 μL was transferred to the each well in the assay plate.
5. The solutions were incubated for 1 h at rt.
6. The plate was read using MET-HTRF® protocol using Pherastar equipment.

### D. Cell Based Assay-Phospho-FAK ELISA

The DuoSet IC ELISA (R&D systems—Cat no. DYC4528-5) provided the basic components required for the development of sandwich ELISAs to measure Focal Adhesion Kinase 1 (FAK) phosphorylated at Y397 in cell lysates. An immobilized capture antibody specific for FAK, also known as PTK2, binds both phosphorylated and unphosphorylated protein. After washing away unbound material, a biotinylated detection antibody specific for FAK phosphorylated at Y397 was used to detect only phosphorylated protein, utilizing a standard Streptavidin-HRP format. The cell line used for the assay was a stable HEK-FAK cell line overexpressing human FL FAK protein, which was generated as follows.

HEK293 cells (ATCC® CRL-1573™, 10801 University Boulevard, Manassas, Va. 20110 USA) were seeded in four 100 mm petri dishes at a seeding density of 1.5×10⁶ cells per plate and incubated overnight at 37° C. in a 5% CO₂ incubator. The transfection mix was prepared as follows. (A) 10 μg DNA (pCDNA4 V5/HisA vector containing Zeocin as mammalian selection marker, procured from Invitrogen) with the full length human FAK gene represented by the nucleotide sequence provided in NCBI Reference Sequence: NM_153831.3, SEQ ID NO: 1:

```
      1 gcgcacgcgc gcgggcccgc gccgacgcag cacggcctcg agggcgcgag cccgcgccgc
    61 cgccgccgcc gccggtcccg gaccactgtg agcccgcggc gtgaggcgtg ggaggaagcg
  121 cggctgctgt cgcccagcgc cgccccgtcg tcgtctgcct tcgcttcacg gcgccgagcc
  181 gcggtccgag cagaactggg gctcccttgc atcttccagt tacaaattca gtgccttctg
  241 cagtttcccc agagctcctc aagaataacg gaagggagaa tatgacagat acctagcatc
  301 tagcaaaata atggcagctg cttaccttga ccccaacttg aatcacacac caaattcgag
  361 tactaagact cacctgggta ctggtatgga acgttctcct ggtgcaatgg agcgagtatt
  421 aaaggtcttt cattattttg aaagcaatag tgagccaacc acctgggcca gtattatcag
  481 gcatggagat gctactgatg tcaggggcat cattcagaag atagtggaca gtcacaaagt
  541 aaagcatgtg gcctgctatg gattccgcct cagtcacctg cggtcagagg aggttcactg
  601 gcttcacgtg gatatgggcg tctccagtgt gagggagaag tatgagcttg ctcacccacc
  661 agaggagtgg aaatatgaat tgagaattcg ttatttgcca aaaggatttc taaaccagtt
  721 tactgaagat aagccaactt tgaatttctt ctatcaacag gtgaagagcg attatatgtt
  781 agagatagct gatcaagtgg accaggaaat tgctttgaag ttgggttgtc tagaaatacg
  841 gcgatcatac tgggagatgc ggggcaatgc actagaaaag aagtctaact atgaagtatt
  901 agaaaaagat gttggtttaa agcgattttt tcctaagagt ttactggatt ctgtcaaggc
  961 caaaacacta agaaaactga tccaacaaac atttagacaa tttgccaacc ttaatagaga
1021 agaaagtatt ctgaaattct ttgagatcct gtctccagtc tacagatttg ataaggaatg
1081 cttcaagtgt gctcttggtt caagctggat tatttcagtg gaactggcaa tcggcccaga
1141 agaaggaatc agttacctaa cggacaaggg ctgcaatccc acacatcttg ctgacttcac
1201 tcaagtgcaa accattcagt attcaaacag tgaagacaag gacagaaaag gaatgctaca
1261 actaaaaata gcaggtgcac ccgagcctct gacagtgacg gcaccatccc taaccattgc
1321 ggagaatatg gctgacctaa tagatgggta ctgccggctg gtgaatggaa cctcgcagtc
1381 atttatcatc agacctcaga aagaaggtga acgggctttg ccatcaatac caaagttggc
1441 caacagcgaa aagcaaggca tgcggacaca cgccgtctct gtgtcagaaa cagatgatta
1501 tgctgagatt atagatgaag aagatactta caccatgccc tcaaccaggg attatgagat
1561 tcaaagagaa agaatagaac ttggacgatg tattggagaa ggccaatttg gagatgtaca
1621 tcaaggcatt tatatgagtc cagagaatcc agctttggcg gttgcaatta aaacatgtaa
1681 aaactgtact tcggacagcg tgagagagaa atttcttcaa gaagccttaa caatgcgtca
1741 gtttgaccat cctcatattg tgaagctgat tggagtcatc acagagaatc ctgtctggat
1801 aatcatggag ctgtgcacac ttggagagct gaggtcattt ttgcaagtaa ggaaatacag
1861 tttggatcta gcatctttga tcctgtatgc ctatcagctt agtacagctc ttgcatatct
1921 agagagcaaa agatttgtac acagggacat tgctgctcgg aatgttctgg tgtcctcaaa
1981 tgattgtgta aaattaggag actttggatt atcccgatat atggaagata gtacttacta
2041 caaagcttcc aaaggaaaat tgcctattaa atggatggct ccagagtcaa tcaattttcg
2101 acgttttacc tcagctagtg acgtatggat gtttggtgtg tgtatgtggg agatactgat
2161 gcatggtgtg aagccttttc aaggagtgaa gaacaatgat gtaatcggtc gaattgaaaa
2221 tggggaaaga ttaccaatgc ctccaaattg tcctcctacc ctctacagcc ttatgacgaa
2281 atgctgggcc tatgacccca gcaggcggcc caggtttact gaacttaaag ctcagctcag
2341 cacaatcctg gaggaagaga aggctcagca agaagagcgc atgaggatgg agtccagaag
2401 acaggccaca gtgtcctggg actccggagg gtctgatgaa gcaccgccca agcccagcag
2461 accgggttat cccagtccga ggtccagcga aggattttat cccagcccac agcacatggt
2521 acaaaccaat cattaccagg tttctggcta ccctggttca catggaatca cagccatggc
2581 tggcagcatc tatccaggtc aggcatctct tttggaccaa acagattcat ggaatcatag
2641 acctcaggag atagcaatgt ggcagcccaa tgtggaggac tctacagtat tggacctgcg
2701 agggattggg caagtgttgc caacccatct gatggaagag cgtctaatcc gacagcaaca
2761 ggaaatggaa gaagatcagc gctggctgga aaaagaggaa agatttctga aacctgatgt
2821 gagactctct cgaggcagta ttgacaggga ggatggaagt cttcagggtc cgattggaaa
2881 ccaacatata tatcagcctg tgggtaaacc agatcctgca gctccaccaa agaaaccgcc
2941 tcgccctgga gctcccggtc atctgggaag ccttgccagc ctcagcagcc ctgctgacag
3001 ctacaacgag ggtgtcaagc ttcagcccca ggaaatcagc ccccctccta ctgccaacct
3061 ggaccggtcg aatgataagg tgtacgagaa tgtgacgggc ctggtgaaag ctgtcatcga
3121 gatgtccagt aaaatccagc cagccccacc agaggagtat gtccctatgg tgaaggaagt
3181 cggcttggcc ctgaggacat tattggccac tgtggatgag accattcccc tcctaccagc
3241 cagcacccac cgagagattg agatggcaca gaagctattg aactctgacc tgggtgagct
3301 catcaacaag atgaaactgg cccagcagta tgtcatgacc agcctccagc aagagtacaa
3361 aaagcaaatg ctgactgctg ctcacgccct ggctgtggat gccaaaaact tactcgatgt
3421 cattgaccaa gcaagactga aaatgcttgg gcagacgaga ccacactgag cctcccctag
3481 gagcacgtct tgctaccctc ttttgaagat gttctctagc cttccaccag cagcgaggaa
3541 ttaaccctgt gtcctcagtc gccagcactt acagctccaa cttttttgaa tgaccatctg
3601 gttgaaaaat ctttctcata taagtttaac cacactttga tttgggttca ttttttgttt
3661 tgtttttttc aatcatgata ttcagaaaaa tccaggatcc aaaatgtggc gtttttctaa
3721 gaatgaaaat tatatgtaag cttttaagca tcatgaagaa caatttatgt tcacattaag
3781 atacgttcta aagggggatg gccaaggggt gacatcttaa ttcctaaact accttagctg
3841 catagtggaa gaggagagca tgaagcaaag aattccagga aacccaagag gctgagaatt
3901 cttttgtcta ccatagaatt attatccaga ctggaatttt tgtttgttag aacacccttc
3961 agttgcaata tgctaatccc actttacaaa gaatataaaa gctatatttt gaagacttga
4021 gttatttcag aaaaaactac agcccttttt gtcttacctg ccttttactt tcgtgtggat
4081 atgtgaagca ttgggtcggg aactagctgt agaacacaac taaaaactca tgtctttttt
4141 cacagaataa tgtgccagtt ttttgtagca atgttatttc tcttggaagc agaaatgctt
4201 tgtaccagag cacctccaaa ctgcattgag gagaagttcc agaaccatcc cctttttcca
4261 tttttatata atttataaag aaagattaaa gccatgttga ctattttaca gccactggag
4321 ttaactaacc cttccttgta tctgtcttcc caggagagaa tgaagcaaaa caggaatttg
4381 gttttctttt gatgtccagt tacaccatcc attctgttaa ttttgaaaaa atataccctc
4441 cctttagttt gttgggggat ataaattatt ctcaggaaga atataatgaa ctgtacagtt
4501 actttgacct attaaaaagg tgttaccagt aaagttcttg ttgtaatatc cttaaaaaaa
4561 a
```

This sequence was inserted using techniques described by the vector manufacturer, that encoded FAK1 isoform A represented by sequence 1052AA, SEQ ID NO: 2:

```
      1 MAAAYLDPNL NHTPNSSTKT HLGTGMERSP GAMERVLKVF HYFESNSEPT TWASIIRHGD
    61 ATDVRGIIQK IVDSHKVKHV ACYGFRLSHL RSEEVHWLHV DMGVSSVREK YELAHPPEEW
  121 KYELRIRYLP KGFLNQFTED KPTLNFFYQQ VKSDYMLEIA DQVDQEIALK LGCLEIRRSY
  181 WEMRGNALEK KSNYEVLEKD VGLKRFFPKS LLDSVKAKTL RKLIQQTFRQ FANLNREESI
  241 LKFFEILSPV YRFDKECFKC ALGSSWIISV ELAIGPEEGI SYLTDKGCNP THLADFTQVQ
  301 TIQYSNSEDK DRKGMLQLKI AGAPEPLTVT APSLTIAENM ADLIDGYCRL VNGTSQSFII
  361 RPQKEGERAL PSIPKLANSE KQGMRTHAVS VSETDDYAEI IDEEDTYTMP STRDYEIQRE
  421 RIELGRCIGE GQFGDVHQGI YMSPENPALA VAIKTCKNCT SDSVREKFLQ EALTMRQFDH
  481 PHIVKLIGVI TENPVWIIME LCTLGELRSF LQVRKYSLDL ASLILYAYQL STALAYLESK
  541 RFVHRDIAAR NVLVSSNDCV KLGDFGLSRY MEDSTYYKAS KGKLPIKWMA PESINFRRFT
  601 SASDVWMFGV CMWEILMHGV KPFQGVKNND VIGRIENGER LPMPPNCPPT LYSLMTKCWA
  661 YDPSRRPRFT ELKAQLSTIL EEEKAQQEER MRMESRRQAT VSWDSGGSDE APPKPSRPGY
  721 PSPRSSEGFY PSPQHMVQTN HYQVSGYPGS HGITAMAGSI YPGQASLLDQ TDSWNHRPQE
  781 IAMWQPNVED STVLDLRGIG QVLPTHLMEE RLIRQQQEME EDQRWLEKEE RFLKPDVRLS
  841 RGSIDREDGS LQGPIGNQHI YQPVGKPDPA APPKKPPRPG APGHLGSLAS LSSPADSYNE
  901 GVKLQPQEIS PPPTANLDRS NDKVYENVTG LVKAVIEMSS KIQPAPPEEY VPMVKEVGLA
  961 LRTLLATVDE TIPLLPASTH REIEMAQKLL NSDLGELINK MKLAQQYVMT SLQQEYKKQM
1021 LTAAHALAVD AKNLLDVIDQ ARLKMLGQTR PH
```

was added to 1.5 mL Opti-MEM® (reduced serum media is a modification of Eagle's Minimum Essential Media, buffered with HEPES and sodium bicarbonate and supplemented with hypoxanthine, thymidine, sodium pyruvate, L-glutamine, trace elements and growth factors commercially available from Invitrogen) and incubated at room temperature for 5 minutes. (B) 30 μL Lipofectamine™ Transfection reagent (Life Technologies) was added to 1.5 mL Opti-MEM® and incubated at room temperature for 5 minutes. Following this, (a) and (b) were mixed and incubated at room temperature for 20 minutes. Media was removed from cells and washed with 1× phosphate buffered saline (PBS) and the transfection mix prepared above was mixed with 3 mL of Opti-MEM®, added to the cells and incubated at 37° C. Four hours later, 6 mL of 20% Dulbecco's Modified Eagle Medium (DMEM) was added to the plates and incubated at 37° C. in a 5% CO₂ incubator. Approximately 72 hours after transfection, cells were subcultured at various dilutions with fresh medium containing 0.8 mg/mL Zeocin™ (Life Technologies). Cells were replenished with selective medium every 3-4 days until cell foci were identified in the FAK transfected cells and most of the cells died in the control plates. Cells from these foci were grown in the presence of selective media and the expression of FAK was confirmed by Western Blotting. Over expression of FAK in the stably-transfected cells was quantified in the cell-based ELISA assay described below by determining the signal window, with a signal window of 2 or above considered to be robust). The HEK-FAK stable cell line thus was maintained in 0.8 mg/mL Zeocin™ (Life Technologies) and used in cell-based ELISA to assess autophosphorylation of FAK.

(i) Materials
1. Phospho-FAK (Y397)-Capture Antibody
2. Phospho-FAK (Y397)-Detection Antibody
3. Streptavidin-HRP
4. Cells used for the assay-HEK over expressing human FL FAK protein

(ii) Solutions
PBS—137 mM NaCl, 2.7 mM KCl, 8.1 mM Na₂HPO₄, 1.5 mM KH₂PO₄, pH 7.2-7.4, 0.2 μm filtered.
Wash Buffer—0.05% Tween20™ in PBS, pH 7.2-7.4
Block Buffer—1% BSA, 0.05% NaN₃, in PBS, pH 7.2-7.4.
IC Diluent #1-1% BSA in PBS, pH 7.2-7.4, 0.2 μm filtered.
IC Diluent #8-1 mM EDTA, 0.5% Triton™ X-100, 5 mM NaF in PBS, pH 7.2-7.4. IC Diluent #8 was also the base buffer for IC Diluent #3, IC Diluent #7, and Lysis.
Buffer #6. Approximately 50 mL of this diluent was required to run the assay on one plate.
IC Diluent #3—1 mM EDTA, 0.5% Triton™ X-100, 5 mM NaF, 1 M urea in PBS, pH 7.2-7.4.
IC Diluent #7—1 mM EDTA, 0.5% Triton™ X-100, 5 mM NaF, 6 M urea in PBS, pH 7.2-7.4.
Lysis Buffer #6—1 mM EDTA, 0.5% Triton™ X-100, 5 mM NaF, 6 M urea, 2.5 mM sodium pyrophosphate, 1 mM activated sodium orthovanadate, Protease inhibitor cocktail, phosphatase inhibitor, PMSF in PBS, pH 7.2-7.4.
Substrate Solution—1:1 mixture of Color Reagent A (H₂O₂) and Color Reagent B (Tetramethylbenzidine, R&D Systems, Catalog # DY999).
Stop Solution—2 N H₂SO₄.

(iii) Reagent Preparation

All reagents were brought to rt before use. Phospho-FAK (Y397) Capture Antibody (Part 843042)—Each vial contained 720 μg/mL of mouse anti-human FAK antibody when reconstituted with 200 μL of PBS. After reconstitution, they were stored at 2-8° C. for up to 30 days or aliquoted and stored at −20° C. in a manual defrost freezer or at −70° C. for up to 3 months. Phospho-FAK (Y397) Detection Antibody (Part 843043)—each vial contained 3.6 μg/mL of biotinylated rabbit anti-human phospho-FAK (Y397) antibody when reconstituted with 1.0 mL of IC Diluent #1. After reconstitution, they were stored at 2-8° C. for up to 30 days or aliquoted and stored at −20° C. in a manual defrost freezer or at −70° C. for up to 3 months. Streptavidin-HRP (Part 890803)—1.0 mL of streptavidin conjugated to horseradish-peroxidase was stored at 2-8° C. and was not frozen.

(iv) Preparation of Samples

The appropriate number of cells was seeded in PDL-coated plates in 100 μL of medium. After 16-18 h, the cells were treated with compound and lysed. The compound of Example 193 was used as reference in this assay.

Cell Lysates—Cells were rinsed two times with PBS, making sure any remaining PBS was removed after the second rinse. The cells were solubilized at 1×10⁷ cells/mL in Lysis Buffer #6 and the samples sat on ice for 15 min. The assay was performed immediately or the samples were stored at −70° C. Before use, the samples were centrifuged at 2000×g for 5 min and the supernatant was transferred to a clean test tube. The sample protein concentration was quantified using a total protein assay. For assaying, the lysates were diluted 6-fold with IC Diluent #8 and further serial dilutions were made in IC Diluent #3. The final concentration of urea in all samples and standards was 1 M prior to addition to the plate.

(v) Plate Preparation
1. The Capture Antibody was diluted to a working concentration of 4.0 μg/mL in PBS, without carrier protein. A 96 well microplate was immediately coated with 100 μL per well of the diluted capture Antibody. The plate was sealed and incubated overnight at rt.
2. Each well was aspirated and washed with Wash Buffer, where the process was repeated two times for a total of 3 washes. Each wash was performed by filling each well with Wash Buffer (400 μL) using a squirt bottle, manifold dispenser or autowasher. Complete removal of liquid at each step was essential for good performance. After the last wash, any remaining Wash Buffer was removed by aspirating or by inverting the plate and blotting it against clean paper towels.
3. The plates were blocked by adding 300 μL of Block Buffer to each well. The samples were incubated at rt for 1-2 h.
4. The aspiration/wash was repeated as in step 2. The plates were then ready for sample addition.

(vi) Assay Procedure
1. 100 μL of sample was added to each well. It was covered with a plate sealer and incubated 2 h at rt.
2. The aspiration/wash as in step 2 of Plate Preparation was repeated.
3. The Detection Antibody was diluted to a working concentration of 100 ng/mL in IC Diluent #1 before use. 100 μL of the diluted Detection Antibody was added to each well. The plate was covered with a new plate sealer and incubated 2 h at rt.
4. The aspiration/wash as in step 2 of Plate Preparation was repeated.
5. Immediately before use, the Streptavidin-HRP was diluted to the working concentration specified on the vial label using IC Diluent #1. 100 μL of the diluted Streptavidin-HRP was added to each well. The plate was incubated for 20 min at rt. The plate was not placed in direct light.
6. The aspiration/wash as in step 2 of the Plate Preparation was repeated.
7. 100 μL of Substrate Solution was added to each well. The plate was incubated for 20 min at room temperature. The plate was not placed in direct light.
8. 50 μL of Stop Solution was added to each well and the plate gently tapped to ensure thorough mixing.
9. The optical density of each well was immediately determined, using a microplate reader set to 450 nm. If wavelength correction was available, it was set to 540 nm or 570 nm. If wavelength correction was not available, the readings were subtracted at 540 nm or 570 nm from the readings at 450 nm. This subtraction corrected for optical imperfections in the plate, readings made directly at 450 nm without correction may be higher and less accurate.

### E. Phospho Src(Y416) Sandwich ELISA

Phospho Src(Y416) Sandwich ELISA kit was obtained from Cell Signaling Tech.

(i) Reagent Preparation
1. All microwell strips were brought to rt before use.
2. 1× Wash Buffer was prepared by diluting 20× Wash Buffer (included in each PathScan® Sandwich ELISA Kit) in MilliQ® water or equivalently purified water.
3. 1× Cell Lysis Buffer from CST #9803: 20 mM Tris (pH 7.5), 150 mM NaCl, 1 mM ethylene diamine tetraacetate (EDTA), 1 mM ethylene glycol-bis(2-aminoethyl)-N,N,N′,N′-tetraacetic acid (EGTA), 1% Triton™ X-100, 2.5 mM sodium pyrophosphate, 1 mM b-glycerophosphate, 1 mM Na₃VO₄, 1 μg/mL leupeptin. This buffer was stored at 4° C. for short-term use (1-2 weeks).

(ii) Preparing Cell Lysates
1. The media was aspirated. The cells were treated by adding fresh media containing inhibitor for 1 h. The compound of Example 214 was used as reference.
2. The cells were harvested under non-denaturing conditions by removing media and rinsing cells once with ice-cold PBS.
3. PBS was removed, 0.5 mL ice-cold 1× Cell Lysis Buffer plus 1 mM phenylmethylsulfonylfluoride (PMSF) was added to each plate (10 cm in diameter) and the plate incubated on ice for 5 min.
4. The cells were scraped off of the plate, transferred to an appropriate tube, and kept on ice.
5. The lysates were sonicated on ice.
6. Microcentrifugation was performed for 10 min at 4° C. and the supernatant transferred to a new tube. The supernatant was the cell lysate and was stored at −80° C. in single-use aliquots.

(iii) Test Procedure
1. After the microwell strips reached rt, the required number of microwells were broken off. The microwells were placed in the strip holder. Unused microwells were resealed and stored at 4° C. immediately.
2. 100 μL of Sample Diluent (supplied in each PathScan® Sandwich ELISA Kit, blue color) was added to a micro centrifuge tube. 100 μL of cell lysate was transferred into the tube and vortexed for a few seconds. The sample applied to the well was diluted 1:1 when the suggested cell lysis buffer was used for cell extraction. Individual data sheets for each kit provided information regarding an appropriate dilution factor for lysates and kit assay results. However, dilution factors were titrated when specific cell lysates are used.
3. 100 μL of each diluted cell lysate were added to the appropriate well. It was then sealed with tape and pressed firmly onto top of microwells. The plate was incubated for 2 h at 37° C. Alternatively, the plate was incubated overnight at 4° C., which gave the best detection of target protein.
4. The tape was gently removed and the wells washed:
   a. The plate contents were discarded into a receptacle.
   b. Each well was washed 4 times with 1× Wash Buffer, 200 μL.
   c. For each wash, plates were struck on fresh towels hard enough to remove the residual solution in each well, but wells were not allowed to completely dry at any time.
   d. The underside of all wells was cleaned with a lint-free tissue.
5. 100 μL of Detection Antibody (green color) was added to each well, sealed with tape and the plate incubated for 1 h at 37° C.
6. The wash procedure was repeated as in Step 4.
7. 100 μL of HRP-linked secondary antibody (red color) was added to each well, sealed with tape and the plate incubated plate for 30 min at 37° C.
8. The wash procedure was repeated as in Step 4.
9. 100 μL of TMB Substrate was added to each well, sealed with tape and the plate incubated for 10 min at 37° C. or 30 min at 25° C.
10. 100 μL of STOP Solution was added to each well and shaken gently for a few seconds. Initial color of positive reaction was blue, which changed to yellow upon addition of STOP Solution.
11. The results were read.
   a. Visual Determination—read within 30 min after adding STOP Solution.
   b. Spectrophotometric Determination—the underside of wells were wiped with a lint-free tissue and absorbance read at 450 nm within 30 min after adding STOP Solution.

Table 3 contains biochemical (FAK, Src, JAK2, AuroraB) and cell-based potency (FAK and Src) for the compounds.

TABLE 3
Biological data of compounds

| | FAK1 | pFAK | Src | pSrc | JAK2 | |
|---|---|---|---|---|---|---|
| | HTRF | EC₅₀ | HTRF | EC₅₀ | HTRF | AuroraB |
| +Example. | IC₅₀ | (μM) | IC₅₀ | (μM) | IC₅₀ | IC₅₀ |
| No. | (μM) | HEK stb | (μM) | Panc1 | (μM) | (μM) |
| 1 | C | | | | B | |
| 2 | C | | | | B | |
| 3 | C | | | | | |
| 4 | C | | C | | B | |
| 5 | A | C | A | C | A | A |
| 6 | D | | | | | |
| 7 | C | | | | | |
| 8 | D | | | | | |
| 9 | D | | | | | |
| 10 | D | | | | | |
| 11 | C | | C | | C | C |
| 12 | C | | C | | B | |
| 13 | D | | | | | |
| 14 | D | | | | | |
| 15 | D | | | | | |
| 16 | D | | | | | |
| 17 | D | | | | | |
| 18 | B | | B | D | A | A |
| 19 | D | | | | | |
| 20 | C | | D | | C | D |
| 21 | B | D | A | D | A | C |
| 22 | D | | | | | |
| 23 | C | | C | | | |
| 24 | B | | C | | | C |
| 25 | D | | | | | |
| 26 | B | D | D | | | C |
| 27 | C | | D | | C | |
| 28 | A | D | A | C | A | B |
| 29 | C | | B | | B | |
| 30 | B | | D | | C | C |
| 31 | A | D | | | C | C |
| 32 | B | | | | C | C |
| 33 | C | | | | | |
| 34 | B | | C | | B | |
| 35 | B | | C | | A | A |
| 36 | B | D | B | D | A | D |
| 37 | D | | | | | |
| 38 | C | | C | | B | |
| 39 | C | | D | | | |
| 40 | C | | D | | | |
| 41 | D | | | | | |
| 42 | C | | C | | B | |
| 43 | B | | B | D | A | B |
| 44 | D | | | | | |
| 45 | D | | D | | | |
| 46 | D | | | | | |
| 47 | D | | | | | |
| 48 | D | | | | | |
| 49 | D | | | | | |
| 50 | B | | D | | B | A |
| 51 | D | | | | | |
| 52 | C | | C | | A | |
| 53 | D | | | | | |
| 54 | C | | D | | D | |
| 55 | C | | D | | D | D |
| 56 | C | | D | | | |
| 57 | D | | | | | |
| 58 | C | | D | | D | D |
| 59 | B | D | D | | D | D |
| 60 | B | | D | | | A |
| 61 | C | | | | | |
| 62 | C | | C | | | |
| 63 | D | | | | | |
| 64 | B | D | B | D | B | D |
| 65 | B | C | A | D | B | D |
| 66 | C | | D | | D | D |
| 67 | D | | | | | |
| 68 | D | | | | | |
| 69 | C | | | | | D |
| 70 | D | | | | | |
| 71 | D | | | | | |
| 72 | D | | | | | |
| 73 | C | | C | | C | D |
| 74 | D | | | | | |
| 75 | B | | C | | C | D |
| 76 | B | | B | D | A | |
| 77 | A | D | C | | B | B |
| 78 | D | | D | | D | |
| 79 | B | | B | D | A | C |
| 80 | C | | D | | D | D |
| 81 | D | | D | | D | |
| 82 | B | D | B | D | C | D |
| 83 | A | | B | | | |
| 84 | B | | C | | C | |
| 85 | C | | D | | | C |
| 86 | C | | D | | | D |
| 88 | D | | | | | |
| 89 | D | | | | | |
| 90 | C | | D | | | A |
| 91 | B | | B | | A | A |
| 92 | C | | | | | |
| 93 | B | | D | | B | A |
| 94 | C | | | | | |
| 95 | B | | C | | A | |
| 96 | C | | | | | C |
| 97 | B | | D | | C | B |
| 98 | B | | D | | B | B |
| 99 | B | | C | | A | |
| 100 | A | | C | | A | |
| 101 | B | | C | | A | |
| 102 | B | | D | | B | A |
| 103 | A | C | B | | A | |
| 104 | B | | D | | | |
| 105 | B | | C | | A | A |
| 106 | A | | C | | A | A |
| 107 | C | | D | | | A |
| 108 | B | | C | | | B |
| 109 | B | | C | | B | |
| 110 | C | | D | | | |
| 111 | A | D | C | | A | |
| 112 | B | | D | | | |
| 113 | B | | D | | D | A |
| 114 | B | | C | | | |
| 115 | A | | B | | | A |
| 116 | B | C | B | C | A | A |
| 117 | B | | C | | B | B |
| 118 | A | | D | | D | A |
| 119 | C | | | | | |
| 120 | C | | C | | | |
| 121 | C | | C | | | |
| 122 | B | | C | | | B |
| 123 | C | | C | | A | A |
| 124 | D | | | | | |
| 125 | C | | C | | | |
| 126 | B | | D | | B | A |
| 127 | C | | C | | | |
| 128 | B | | C | | | |
| 130 | B | | D | | | |
| 131 | B | C | C | | C | C |
| 132 | C | | D | | | |
| 133 | B | C | C | | | |
| 134 | C | | C | | | |
| 135 | C | | C | | C | B |
| 136 | A | C | A | | | A |
| 137 | A | | B | D | | A |
| 138 | C | | D | | | |
| 139 | A | | C | | B | A |
| 140 | B | | C | | | |
| 141 | C | | D | | | |
| 142 | A | D | D | D | C | B |
| 143 | C | | D | | | |
| 144 | C | | D | | | |
| 145 | B | | C | | | |
| 146 | D | | D | | | |
| 147 | B | | C | | | |
| 148 | B | | D | | | C |
| 149 | B | | B | | | |
| 150 | B | | C | | | |
| 151 | A | | B | D | | |
| 152 | A | D | D | | B | B |
| 154 | B | D | D | | D | D |
| 155 | C | | D | | | C |
| 156 | A | | C | | | |
| 157 | C | | C | | | |
| 158 | B | | B | | | |
| 159 | C | | | | | |
| 160 | B | C | D | D | C | B |
| 161 | B | | C | | | B |
| 162 | B | C | B | | | B |
| 163 | C | | | | | |
| 164 | B | | B | | | A |
| 165 | B | | C | | A | A |
| 166 | B | | B | | | A |
| 167 | C | | D | | | C |
| 168 | B | | C | | | |
| 169 | A | | A | D | | A |
| 170 | B | | C | | | |
| 171 | B | | C | | C | |
| 172 | B | | B | | | B |
| 173 | B | | B | C | | A |
| 174 | B | | B | | | |
| 175 | C | | C | | | |
| 176 | B | C | B | D | A | A |
| 177 | B | | C | | | |
| 178 | B | | D | | B | A |
| 179 | B | | B | | | |
| 180 | A | C | C | | A | A |
| 181 | B | | C | | | |
| 182 | B | | B | | | |
| 183 | D | | D | | | |
| 184 | A | C | B | | A | A |
| 185 | A | D | B | C | A | A |
| 186 | B | | B | | | C |
| 187 | A | C | C | | | |
| 188 | C | | D | | | |
| 189 | B | | C | | | A |
| 190 | D | | D | | | |
| 191 | C | D | C | D | D | B |
| 192 | C | | D | | | |
| 193 | A | B | B | C | A | A |
| 194 | A | B | A | C | A | A |
| 195 | B | D | B | A | A | B |
| 196 | A | B | A | C | | A |
| 197 | B | C | A | B | A | A |
| 198 | A | A | B | C | B | A |
| 199 | A | | B | D | | A |
| 200 | B | C | B | C | | A |
| 201 | B | D | C | D | A | A |
| 202 | B | D | A | D | A | |
| 203 | B | C | A | C | | A |
| 204 | A | D | B | | A | A |
| 205 | A | B | B | | A | A |
| 206 | B | C | A | C | A | A |
| 207 | B | D | B | | | |
| 208 | C | D | C | | A | |
| 209 | A | C | A | | | |
| 210 | B | C | C | | D | A |
| 211 | A | B | B | C | | A |
| 212 | A | C | A | | | A |
| 213 | A | B | A | C | A | A |
| 214 | A | B | A | C | A | |
| 215 | A | D | A | | | |
| 216 | A | D | A | C | | A |
| 217 | B | C | B | C | B | C |
| 218 | A | C | B | B | | A |
| 219 | A | C | A | | B | A |
| 220 | B | C | A | C | A | A |
| 221 | B | D | C | | | |
| 222 | A | C | B | B | B | B |
| 223 | A | | A | | | |
| 224 | B | D | A | B | A | A |
| 225 | A | D | C | | | |
| 226 | C | | D | | | B |
| 227 | D | | D | | | |
| 228 | A | C | A | | B | |
| 229 | B | D | A | B | | B |
| 230 | A | B | B | | | A |
| 231 | A | C | B | | B | A |
| 232 | B | D | A | C | | A |
| 233 | C | D | B | | | |
| 234 | C | | D | | | |
| 235 | B | C | C | | A | A |
| 236 | D | | C | | | |
| 237 | C | | C | | | |
| 238 | D | | C | | | |
| 239 | C | | B | | | B |
| 240 | C | | B | | | |
| 241 | C | | A | B | | C |
| 242 | B | D | B | B | | A |
| 243 | B | D | B | B | A | A |
| 244 | A | B | B | C | A | A |
| 245 | B | D | A | B | | A |
| 246 | A | B | B | B | A | A |
| 247 | A | A | B | C | | A |
| 248 | A | C | C | | A | A |
| 249 | B | | B | | | B |
| 250 | B | C | B | C | | A |
| 251 | B | | B | D | | A |
| 252 | C | | B | | | B |
| 253 | A | D | B | D | A | A |
| 254 | B | D | B | | | |
| 255 | C | | B | A | | |
| 256 | A | | B | D | | A |
| 257 | A | B | B | | | A |
| 258 | B | | B | D | | A |
| 259 | A | | B | C | | A |
| 260 | A | D | B | | A | A |
| 261 | A | B | A | B | | A |
| 262 | A | B | A | D | B | A |
| 263 | D | | | | | |
| 264 | A | D | A | | A | A |
| 265 | A | D | A | | A | A |
| 266 | A | D | B | | | A |
| 267 | C | | C | | | |
| 268 | A | B | B | D | A | A |
| 269 | B | | C | | | A |
| 270 | B | D | C | C | | A |
| 271 | C | D | A | A | | B |
| 272 | A | C | B | C | A | A |
| 273 | A | B | C | | D | A |
| 274 | C | | C | | | |
| 275 | C | | B | B | | C |
| 276 | D | | C | | | |
| 277 | C | | D | | | |
| 278 | C | | A | B | | A |
| 279 | C | | B | | | |
| 280 | B | C | C | | | B |
| 281 | C | | C | | | |
| 282 | A | C | C | | D | A |
| 283 | C | | A | B | | A |
| 284 | A | B | B | C | | A |
| 285 | A | C | B | C | | |
| 286 | B | C | D | | D | A |
| 287 | C | | C | | | |
| 288 | B | | D | | | |
| 289 | C | | C | | | |
| 290 | A | C | B | C | A | A |
| 291 | B | D | A | B | A | A |
| 292 | B | D | B | | | |
| 293 | A | C | C | | D | A |
| 294 | A | B | B | C | B | A |
| 295 | B | D | A | | | |
| 296 | A | B | C | | D | A |
| 297 | C | | C | | | |
| 298 | A | B | B | D | B | A |
| 299 | C | | A | B | | A |
| 300 | B | D | C | | D | A |
| 301 | A | D | A | C | | |
| 302 | C | | B | C | | |
| 303 | A | A | B | D | A | A |
| 304 | A | C | B | C | A | A |
| 305 | B | C | C | | | |
| 306 | B | D | C | | A | A |
| 307 | A | C | B | D | A | A |
| 308 | B | D | C | | B | B |
| 309 | C | | C | | | |
| 310 | A | B | B | D | A | A |
| 311 | A | B | A | C | A | A |
| 312 | A | A | B | D | B | A |
| 313 | B | B | C | | C | C |
| 314 | A | C | C | C | A | A |
| 315 | B | C | D | | | A |
| 316 | A | C | B | D | | A |
| 317 | B | D | C | D | | |
| 318 | A | D | B | D | B | A |
| 319 | A | C | C | | A | A |
| 320 | B | D | B | B | A | B |
| 321 | A | B | C | | C | A |
| 322 | A | C | C | | B | B |
| 323 | A | C | C | | C | A |
| 324 | A | C | C | | | |
| 325 | A | C | C | | C | B |
| 326 | A | C | B | B | | A |
| 327 | A | C | D | | D | A |
| 328 | A | B | C | | D | A |
| 329 | A | C | D | | D | A |
| 330 | A | A | D | | D | A |
| 331 | A | B | B | D | B | A |
| 332 | A | A | A | B | C | A |
| 333 | A | C | B | C | A | A |
| 334 | A | B | B | D | C | A |
| 335 | B | D | C | D | | A |
| 336 | A | C | D | | D | B |
| 337 | A | C | D | | D | C |
| 338 | A | A | D | | D | B |
| 339 | A | C | C | | C | A |
| 340 | A | B | C | | C | A |
| 341 | B | D | C | | | |
| 342 | B | D | D | | | A |
| 343 | A | C | D | | C | A |
| 344 | B | D | C | | B | A |
| 345 | A | B | D | | | A |
| 346 | A | B | C | | | A |
| 347 | A | B | D | | D | B |
| 348 | A | C | D | | D | B |
| 349 | B | D | D | | | |
| 350 | A | C | C | D | | |
| 351 | A | C | C | | | B |
| 352 | A | C | C | | | |
| 353 | A | C | C | | | A |
| 354 | B | C | D | | | |
| 355 | B | D | B | C | | A |
| 356 | A | B | D | | D | A |
| 357 | A | C | B | C | A | A |
| 358 | D | D | D | | D | C |
| 359 | A | C | D | | | |
| 360 | A | C | D | | | C |
| 361 | B | C | D | | | C |
| 362 | D | | D | | | |
| 363 | B | C | D | | | C |
| 364 | B | C | D | | | |
| 365 | B | D | D | | | C |
| 366 | B | C | D | | | D |
| 367 | B | C | C | | | A |
| 368 | B | C | C | | | |
| 369 | B | D | D | | | A |
| 370 | B | C | D | | | B |
| 371 | B | D | D | | | |
| 372 | B | D | C | C | | B |
| 373 | A | C | C | | | |
| 374 | A | C | D | | | |
| 375 | B | C | D | | | |
| 376 | A | C | C | | | |
| 377 | B | C | C | | | |
| 378 | C | | C | | | A |
| 379 | B | C | C | | | |
| 380 | D | | B | B | | C |
| 381 | A | C | C | | | A |
| 382 | B | C | D | | | C |
| 383 | B | C | D | | | A |
| 384 | B | C | D | | | C |
| 385 | A | C | D | | | C |
| 386 | A | A | D | | | B |
| 387 | A | A | D | | | C |
| 388 | D | | D | | | |
| 389 | A | C | B | C | | A |
| 390 | A | C | B | C | | A |
| 391 | A | B | B | B | D | A |
| 392 | C | D | C | | | A |
| 393 | B | C | D | | | |
| 394 | B | D | | | | |
| 395 | A | B | | | | A |
| 396 | A | B | D | | | C |
| 397 | B | D | D | | | |
| 398 | C | D | D | | | A |
| 399 | B | C | C | | | C |
| 400 | A | A | D | | | B |
| 401 | A | B | D | | | A |
| 402 | C | | D | | | |
| 403 | A | B | B | C | | C |
| 404 | C | | C | | | D |
| 405 | A | C | B | D | | |
| 406 | A | C | B | D | | |
| 407 | A | B | D | | | C |
| 408 | B | | B | D | | A |
| 409 | A | C | C | | | |
| 410 | A | C | B | C | | A |
| 411 | A | C | C | D | | |
| 412 | A | B | B | D | | A |
| 413 | A | B | C | C | | A |
| 414 | A | C | D | | | |
| 415 | B | C | C | | | |
| 416 | B | C | C | | | |
| 418 | A | C | D | | | B |
| 419 | C | D | D | | | |
| 420 | B | C | C | C | | B |
| 421 | A | D | B | C | | |
| 422 | B | D | C | | | |
| 423 | A | C | B | C | | A |
| 424 | A | C | D | | | C |
| 425 | A | C | C | | | |
| 426 | A | B | D | | | |
| 427 | A | C | B | | | A |
| 428 | A | B | B | C | | A |
| 429 | A | C | A | C | | A |
| 430 | A | C | D | | | |
| 431 | A | B | B | C | | A |
| 432 | A | C | C | D | | |
| 433 | C | | D | | | |
| 434 | A | C | B | D | | A |
| 435 | C | D | C | | | |
| 436 | A | C | B | | | |
| 437 | D | | D | | | D |
| 438 | A | C | B | B | | |
| 439 | A | C | C | | | |
| 440 | A | C | C | | | A |
| 441 | A | C | D | | | C |
| 442 | A | C | B | | | |
| 443 | A | C | C | | | |
| 444 | A | A | D | | | B |
| 445 | B | C | C | | | C |
| 446 | A | B | D | | D | D |
| 447 | A | C | A | B | | |
| 448 | A | B | B | B | A | A |
| 449 | A | B | B | D | | |
| 450 | A | C | C | | | |
| 451 | A | A | D | | | C |
| 452 | B | D | B | D | | |
| 453 | B | C | B | D | | C |
| 454 | A | B | B | D | | |
| 455 | C | D | D | | | |
| 456 | B | D | | | | |
| 457 | B | C | B | D | | |
| 458 | A | C | B | D | | |
| 459 | B | C | B | C | | |
| 460 | A | C | D | | | |
| 461 | C | | C | | | D |
| 462 | A | B | B | C | | A |
| 463 | B | C | B | C | | A |
| 464 | B | D | C | | | C |
| 465 | A | C | B | D | | A |
| 466 | C | | C | | | D |
| 467 | A | C | B | B | A | A |
| 468 | A | C | B | B | A | A |
| 469 | C | | C | | | |
| 470 | A | C | C | C | | B |
| 471 | B | C | B | B | A | B |
| 472 | A | C | C | B | C | A |
| 473 | A | C | C | D | | B |
| 474 | D | | C | | | C |
| 475 | B | C | B | B | A | A |
| 476 | A | C | B | D | | |
| 477 | C | | D | | | B |
| 478 | A | C | C | | | A |
| 479 | B | D | C | | | A |
| 480 | A | C | C | | | A |
| 481 | B | C | B | B | A | A |
| 482 | B | C | B | C | | A |
| 483 | C | | C | C | | B |
| 484 | A | C | B | B | | B |
| 485 | A | C | B | C | | A |
| 486 | A | C | B | D | | A |
| 487 | B | C | B | B | | A |
| 488 | B | C | B | C | | A |
| 489 | C | | C | | | B |
| 490 | A | C | B | C | | A |
| 491 | A | C | A | B | | |
| 492 | A | C | B | C | | |
| 493 | A | C | D | | | |
| 494 | A | B | C | D | C | B |
| 495 | A | C | B | B | | B |
| 496 | A | C | A | B | | B |
| 497 | B | D | B | B | | B |
| 498 | C | | C | | | D |
| 499 | A | B | B | C | | B |
| 500 | A | C | B | C | D | C |
| 501 | C | | C | | | C |
| 502 | C | | C | | | |
| 503 | B | C | D | | | |
| 504 | A | C | D | | | D |
| 505 | A | C | C | | | |
| 506 | A | D | B | C | | |
| 508 | B | D | C | D | A | |
| 509 | C | | C | | | |
| 510 | B | | D | | | D |
| 511 | B | C | D | | | |
| 512 | B | D | D | | | |
| 513 | A | C | B | D | A | A |
| 514 | A | D | D | | | |
| 515 | B | D | C | C | A | B |
| 516 | C | D | B | D | | |
| 518 | B | C | C | | | C |
| 519 | B | D | A | B | | B |
| 520 | B | D | B | C | | |
| 521 | B | C | B | C | | B |
| 522 | B | D | B | C | | C |
| 523 | A | C | B | | | B |
| 524 | B | C | C | | | D |
| 525 | C | | B | C | | C |
| 526 | A | D | B | C | | B |
| 527 | B | C | B | C | | B |
| 528 | D | | D | | | D |
| 529 | A | C | C | | | B |
| 530 | B | D | B | C | | C |
| 531 | A | C | B | C | | B |
| 532 | C | | D | | | D |
| 533 | C | | B | C | | C |
| 534 | B | D | C | | | D |
| 535 | B | D | B | C | | B |
| 536 | A | D | B | D | | B |
| 537 | B | D | C | C | | |
| 538 | B | D | B | C | | C |
| 539 | A | C | B | D | | |
| 540 | B | D | C | C | | C |
| 542 | C | | C | | | |
| 543 | C | | C | | | |
| 544 | C | | C | | | |
| 545 | A | C | C | | | A |
| 546 | A | C | B | D | | A |
| 547 | B | D | A | B | | A |
| 548 | B | D | B | C | | B |
| 549 | C | D | C | | | |
| 550 | A | D | C | | | A |
| 551 | B | C | C | | | A |
| 552 | B | C | C | | | A |
| 553 | C | | C | | | B |
| 554 | B | C | C | | | D |
| 557 | B | C | C | | | A |
| 558 | D | | D | | | D |
| 559 | B | D | B | D | | B |
| 560 | B | D | C | | | B |
| 561 | B | D | B | C | | C |
| 562 | B | C | B | C | | B |
| 563 | B | C | B | C | | C |
| 564 | B | C | C | | | D |
| 565 | C | | D | | | |
| 566 | A | C | C | | | B |
| 567 | B | C | C | | | B |
| 568 | B | D | B | C | | B |
| 569 | B | C | C | | | B |
| 570 | B | C | C | | | B |
| 571 | A | C | B | C | | B |
| 572 | A | C | C | | | B |
| 573 | B | C | C | | | |
| 574 | C | | C | | | |
| 575 | B | C | B | C | | B |
| 576 | B | D | D | | | |
| 577 | B | C | B | C | | |
| 578 | C | | C | | | |
| 579 | C | | C | | | |
| 580 | C | D | C | | | |
| 581 | B | C | C | | | |
| 582 | C | | C | | | |
| 583 | D | | D | | | |
| 584 | C | | B | B | | |
| 585 | D | | D | | | |
| 586 | C | | C | | | |
| 587 | C | | C | | | |
| 588 | A | C | C | | | B |
| 589 | B | D | C | | | |
| 590 | A | C | B | C | | B |
| 591 | B | C | B | C | | B |
| 592 | C | | C | | | |
| 593 | C | | C | | | |
| 594 | B | C | B | B | | C |
| 595 | A | B | B | | | A |
| 596 | C | | C | | | |
| 597 | C | | C | | | |
| 598 | B | C | B | C | | A |
| 599 | C | | C | | | |
| 600 | C | | B | C | | B |
| 601 | C | | B | C | | |
| 602 | B | C | C | | | A |
| 603 | B | C | D | | | C |
| 604 | C | | C | | | |
| 605 | B | D | C | D | | B |
| 606 | A | C | B | | | A |
| 607 | C | | C | | | |
| 608 | B | C | C | | | A |
| 609 | C | | C | | | |
| 610 | B | C | C | | | A |
| 611 | A | C | D | | | C |
| 612 | C | | C | | | |
| 613 | B | D | C | | | |
| 614 | B | C | C | | | A |
| 615 | B | D | B | | | A |
| 616 | C | | D | | | |
| 617 | B | B | C | C | | C |
| 618 | A | C | B | | | A |
| 619 | B | C | C | | | |
| 620 | C | C | C | | | |
| 621 | B | B | C | | | C |
| 622 | B | C | C | | | |
| 623 | B | D | D | | | |
| 624 | C | | C | | | |
| 625 | B | C | D | | | |
| 626 | B | C | C | | | |
| 627 | B | C | D | | | B |
| 628 | B | C | D | | | D |
| 629 | B | C | D | | | D |
| 630 | B | C | C | | | C |
| 631 | B | C | C | | | A |
| 632 | A | B | C | | | A |
| 633 | C | | C | | | |
| 634 | B | B | D | | | D |
| 635 | A | B | D | | | D |
| 636 | C | | C | | | |
| 637 | C | | C | | | |
| 638 | A | C | C | | | |
| 639 | B | C | C | | | |
| 640 | B | C | C | | | |
| 641 | C | | B | | | |
| 642 | B | C | C | | | |
| 643 | B | C | B | | | B |
| 644 | B | C | B | | | |
| 645 | B | D | B | | | |
| 647 | B | B | B | | | B |
| 648 | B | C | C | | | D |
| 649 | B | C | B | | | |
| 650 | A | | D | | | |
| 651 | B | | C | | | |
| 652 | C | C | C | | | |
| 653 | B | C | C | | | |
| 654 | C | | C | | | |
| 655 | B | C | C | | | |
| 656 | B | | C | | | |
| 657 | | | | | | |
| 658 | | | | | | |
| 659 | B | D | C | D | | |
| 660 | B | C | C | | | C |
| 661 | A | B | C | C | | B |
| 662 | A | C | C | C | | A |
| 663 | B | D | B | C | | C |
| 664 | B | C | C | | | C |
| 665 | A | C | B | C | | A |
| 666 | B | D | B | C | | B |
| 667 | B | D | C | | | |
| 668 | C | | C | D | | |
| 669 | B | D | C | D | | B |
| 670 | B | C | B | B | | B |
| 671 | C | | D | | | |
| 672 | B | B | C | | | B |
| 673 | 0A | C | D | | | |
| 674 | B | C | C | | | |
| 675 | A | B | C | | | A |
| 676 | B | C | C | D | | A |
| 677 | C | D | C | | | B |
| 678 | C | C | C | C | | C |
| 679 | B | C | C | C | | B |
| 680 | B | D | C | | | B |
| 681 | B | C | B | B | | B |
| 682 | D | | C | | | |
| 683 | C | | C | | | |
| 684 | B | D | C | | | B |
| 685 | C | | D | | | |
| 686 | B | C | C | | | C |
| 687 | B | C | D | | | C |
| 688 | B | D | C | | | B |
| 689 | C | | D | | | |
| 690 | C | C | C | C | | |
| 691 | A | B | C | | | A |
| 692 | B | C | C | C | | A |
| 693 | B | C | C | C | | C |
| 694 | B | D | C | C | | C |
| 695 | A | C | B | C | | A |
| 696 | B | D | C | | | |
| 697 | B | C | C | | | |
| 698 | B | D | C | | | |
| 699 | C | D | D | | | |
| 700 | A | B | C | C | | B |
| 701 | C | D | D | | | |
| 702 | A | B | C | | | C |
| 703 | A | B | C | | | B |
| 704 | A | B | C | | | B |
| 705 | A | B | C | B | | B |
| 706 | A | A | C | C | | B |
| 707 | B | D | D | | | C |
| 708 | B | C | B | B | | B |
| 709 | A | B | C | C | | B |
| 710 | A | C | C | C | | |
| 711 | D | | D | | | |
| 712 | A | B | C | D | | A |
| 713 | A | B | D | | | |
| 714 | C | | D | | | |
| 715 | A | B | C | | | C |
| 716 | C | | C | | | |
| 717 | A | B | C | | | |
| 718 | C | | C | | | |
| 719 | C | | D | | | |
| 720 | C | | C | | | |
| 721 | C | | C | | | |
| 722 | C | C | D | | | |
| 723 | C | D | C | | | |
| 724 | C | | C | | | |
| 725 | C | | C | | | |
| 726 | B | D | C | | | |
| 727 | B | C | C | | | |
| 728 | C | | D | | | |
| 729 | D | | D | | | |
| 730 | D | | D | | | |
| 731 | C | | D | | | |
| 732 | D | | D | | | |
| 733 | B | C | C | | | B |
| 734 | D | | D | | | |
| 735 | C | | C | | | |
| 736 | B | C | B | C | | |
| 737 | C | D | C | | | |
| 738 | B | B | C | | | |
| 739 | B | C | C | | | |
| 740 | B | D | D | | | D |
| 741 | D | | D | | | |
| 742 | C | | D | | | |
| 743 | B | | D | | | |
| 744 | A | B | B | | | B |
| 745 | C | D | C | | | |
| 746 | C | | D | | | |
| 747 | C | | C | | | |
| 748 | B | C | C | | | |
| 749 | B | C | C | | | C |
| 750 | A | C | C | D | | B |
| 751 | B | C | C | | | A |
| 752 | B | C | C | | | |
| 753 | C | | C | D | | |
| 754 | C | | C | | | |
| 755 | C | D | D | | | |
| 756 | C | | D | | | |
| 757 | B | C | C | | | |
| 758 | B | C | C | | | |
| 759 | A | B | B | C | | B |
| 760 | C | | D | | | |
| 761 | A | B | C | C | | B |
| 762 | B | D | D | | | D |
| 763 | A | B | B | B | | A |
| 764 | C | | D | | | |
| 765 | C | | C | | | |
| 766 | A | B | B | C | | |
| 767 | C | | D | | | |
| 768 | C | D | C | | | B |
| 769 | A | B | C | | | |
| 770 | B | C | C | C | | |
| 771 | B | C | D | | | |
| 772 | C | | D | | | C |
| 773 | C | | C | C | | |
| 774 | B | C | | | | B |
| 775 | B | C | C | | | |
| 776 | B | D | B | B | | |
| 777 | A | C | C | | | A |
| 778 | A | B | B | B | | B |
| 779 | C | | D | | | |
| 780 | A | C | B | C | | A |
| 781 | C | | D | | | |
| 782 | A | C | C | B | | B |
| 783 | B | C | C | | | |
| 784 | D | | D | | | |
| 785 | D | | D | | | |
| 786 | A | B | D | | | |
| 787 | D | | D | | | |
| 788 | D | | D | | | |
| 789 | A | C | C | | | C |
| 790 | C | | D | | | |
| 791 | C | | D | | | |
| 792 | A | B | B | C | | |
| 793 | A | B | B | | | |
| 794 | A | C | B | B | | |
| 795 | C | | C | B | | |
| 796 | A | B | B | B | | |
| 797 | C | | C | | | |
| 798 | C | | C | | | |
| 799 | A | C | B | B | | |
| 800 | A | C | B | B | | |
| 801 | A | C | B | B | | |
| 802 | A | | B | | | |
| 803 | B | | B | | | |
| 804 | C | | C | | | |
| 805 | A | | B | | | |
| 806 | A | | C | | | |
| 807 | A | | C | | | |
| 808 | B | | C | | | |
| 809 | A | | B | | | |
| 810 | A | | C | | | |
| 887 | B | D | B | B | | |
| 888 | C | | C | | | |
| 892 | A | C | C | | | |
| 899 | A | B | B | C | | |
| 920 | A | C | B | B | | |
| 941 | A | C | B | B | | |
| 942 | A | B | B | C | | |
| 951 | C | | B | | | |
| 953 | C | | C | | | |
| 958 | C | | B | | | |
| 977 | A | C | B | C | | |
| 978 | A | B | B | C | | |
| 979 | B | C | C | | | |
| 980 | A | C | C | | | |
| 981 | A | C | B | C | | |
| 982 | B | D | C | | | |
| 983 | A | B | B | C | | |
| 984 | B | C | C | | | |
| 985 | C | | C | | | |
| 986 | A | | C | | | |
| 987 | B | | C | | | |

### FAK, Src, JAK2, and AuroraB IC₅₀<20 nM (A); 21-100 nM (B); 101-1000 nM (C); >1000 nM (D)

pFAK and pSrc EC₅₀<20 nM (A); 21-100 nM (B); 101-1000 nM (C); >1000 nM (D)

### F. Tumor Implantation and Tumor Growth Monitoring

(i) Reagent
   Matrigel—BD Biosciences—Cat #354234
(ii) Animals: Athymic nude mice (Foxn1 nu/nu) from Harlan
(iii) Procedure
   The cells listed below were grown up-to 70% confluency, Passage #<10
      Calu-6 (ATCC, HTB-56)
      HT-29 (ATCC, HTB-38)
      BxPC3 (ATCC, CRL-1687)
   The cells were trypsinized and counted.
   The required amount of cells (2-6×10⁶ cells/animal in 100 μL of medium) was calculated.
   The Matrigel was thawed properly and kept it on the ice while using.
   The cells were mixed in media with matrigel in 1:1 proportion—200 μL/animal—(100 μL matrigel+100 μL media with required amount of cells).
   The animal was properly restrained, the area swabbed with alcohol and injected subcutaneously at the right flank region of nude mice.
   Tumor growth was monitored by tumor volume measurements taken during course of the study. The tumor growth was monitored after 7 days of injection and measurements were taken after visual inspection of growth at the site of injection.
   In vivo target inhibition experiments were planned based on tumor volume, where expected tumor volume on an average was 100-150 mm³. Animals showing tumor growth were randomized and divided into groups.
   Compounds were formulated with different vehicle formulations and dosed according to the study plan.

### G. Western Blotting Procedure for Phosphor-Protein Detection

### (i) Preparation of Tumor Lysates

1. The tumor was harvested.
2. The tumor piece was homogenized in 2 mL RIPA buffer.
3. The homogenate was spun at 10,000 rpm for 5 min at 4° C.
4. The supernatant was transferred to a new microcentrifuge tube and the pellet discarded.
5. The protein concentration (Bradford assay) was determined using BSA standards as given below

BSA (1 mg/mL)—standard, samples and Bradford reagent was added in the below-mentioned pattern.

| | | Bradford | | |
|---|---|---|---|---|
| MilliQ ® | BSA | undiluted | Protein Conc. | |
| water | (μL) | (μL) | achieved | |
| 160 | 0 | 40 | Blank | |
| 158 | 2 | 40 | 2 | μg |
| 156 | 4 | 40 | 4 | μg |
| 152 | 8 | 40 | 8 | μg |
| 148 | 12 | 40 | 12 | μg |

| | | | |
|---|---|---|---|
| Sample | | | |
| 156 | 4 | 40 | unknown |

### (ii) Preparation of Gel

1. The glass plates and spacers (1.5 mm thick) were assembled.
2. The 10% separation gel was prepared as follows:

| | | |
|---|---|---|
| MilliQ ® water | 4.1 | mL |
| 1.5M Tris-Cl pH 8.8 | 2.5 | mL |
| 20% SDS | 50 | μL |
| 30% Acrylamide/bis soln. | 3.3 | mL |
| 10% Ammonia per sulfate (APS) | 50 | μL |
| Temed | 10 | μL |

3. The resolving gel was poured to about 1 cm below the wells of the comb.
4. It was sealed with 1 mL of MilliQ® water. When gel had set, the water was poured off.
5. The 4% stacking gel was prepared as follows:

| | | |
|---|---|---|
| MilliQ ® water | 3.075 | mL |
| 0.5M Tris-cl pH 8.8 | 1.25 | mL |
| 20% SDS | 25 | μL |
| 30% Acrylamide/bis soln. | 0.67 | mL |
| 10% Ammonia per sulfate (APS) | 25 | μL |
| Temed | 5 | μL |

6. The stacking gel (˜5 mL) was poured and the comb was inserted immediately.
7. When the stacking gel had set, it was placed in gel rig and immersed in buffer.
8. Prior to running the gel, the wells were flushed out thoroughly with running buffer.
   (iii) Running the Gel
1. 50 μg protein was aliquoted into in each tube.
2. An appropriate amount of 5× Sample loading buffer was added.
3. Samples were boiled for 10 min.
4. Samples were cooled at RT for 5 min.
5. Flash spin was used to bring down condensation prior to loading gel.
6. 5 μL Bio-Rad Kaleidoscope Prestained Standards #161-0324 were was used as a marker.
7. The gel was run at 100 V for about 1.5 h.
   (iv) iBlot® Dry Blotting System

The iBlot® Dry Blotting System efficiently and reliably blots proteins from polyacrylamide gels in 7 min or less. The iBlot® device uses disposable blotting stacks with integrated nitrocellulose or PVDF membranes. The top and bottom stacks contain the necessary buffers. The bottom stack includes an integrated 0.2 μm nitrocellulose or PVDF membrane.
1. The membrane was wetted on the bottom stack with milliQ® water.
2. The gel(s) was placed on the membrane.
3. The Whatman paper (provided in the kit) was wetted and placed on top of the gel.
4. The top stack was placed and the “sandwich” assembled in the iBlot® device.
5. Transfer occurred in/within 7 min.
6. Once transferred, membranes were immersed in blocking buffer (5% skimmed milk powder in 1×PBS containing 0.05% Tween20™) and blocked for 45 mins to 1 h.

### (v) Antibodies and Detection

1. The blot with was incubated with primary antibodies (1:1000) diluted in 5% BSA overnight at 4° C. on a shaker.
2. The blot was washed 3×10 min with 0.05% Tween20™ in PBST.
3. The blot was incubated with secondary antibody, anti-rabbit HRP (Sigma Aldrich, 1:2000) diluted in 2.5% milk for 45 min at rt on a shaker.
4. The blot was washed 3×10 min with 0.05% Tween20™ in PBST.
5. Detection was performed with Amersham ECL kit and it developed using LAS 4000 Imager at different exposures.

### (vi) Buffers for Western Blotting

### Lysis Buffer:

| | |
|---|---|
| 10 mM Tris pH 7.4 | 100 mM NaCl |
| 1 mM EDTA | 1 mM EGTA |
| 1 mM NaF | 20 mM Na₄P₂O₇ |
| 2 mM Na₃VO₄ | 1% Triton ™ X-100 |
| 10% Glycerol | 0.1% SDS |
| 0.5% Deoxycholate | |

Just before using add: to 1 mL

10 μL of Protease inhibitor cocktail

10 μL of phosphatase inhibitor cocktail

2× Laemmli sample loading buffer:

| | |
|---|---|
| 0.5M Tris-Cl, pH 6.8 | 0.02% Bromophenol blue |
| 20% (v/v) Glycerol | 1 mL of beta-mercaptoethanol |
| 10% (w/v) SDS | |

8× Resolving gel buffer: 100 mL

0.8 g SDS (add last)

36.3 g Trizma base (3 M)

pH adjusted to 8.8 with concentrated HCl

4× Stacking gel buffer: 100 mL

0.4 g SDS (add last)

6.05 g Trizma base (0.5 M)

pH adjusted to 6.8

10× Running buffer: 1 L

30.3 g Trizma base (0.25 M)

144 g Glycine (1.92 M)

10 g SDS (1%)—add last

The pH was not adjusted.

10× Blotting buffer: 1 L

30.3 g Trizma base (0.25 M)

144 g Glycine (1.92 M)

The pH was 8.3 and was not adjusted

To make 2 L of 1× Blotting buffer:

400 mL Methanol

200 mL 10× Blotting buffer

1400 mL water

Blocking buffer: 50 mL (Freshly prepared)

2.5 g of skimmed non-fat milk powder was added to 1×PBS containing 0.05% Tween20™.

### H. G2M Abrogation/Cell Cycle Arrest Studies

### (i) Sample Preparation for Cell Cycle Analysis by Flow Cytometry

1. Calu-6 (ATCC, HTB-56)/HT-29 (ATCC, HTB-38) cells were seeded at a density of 0.1-0.2×10⁶ per well in a 24 well plate.
2. Approximately 7 h after seeding, the cells were treated with respective concentrations of the compounds. The Pfizer aurora inhibitor was used as reference.
3. After 16 h, media was removed from wells, 500 μL of Trypsin added, the solution neutralized with 500 μL media, and collected in 15 mL falcon tubes.
4. Cells were harvested 16 h after treatment and centrifuged at 1500 rpm for 5 min.
5. The cell pellet was washed in 1×PBS and the cells were resuspended in 200-300 μL Propidium Iodide (PI) buffer before acquiring. Preferably, PI buffer was prepared fresh every time and stored in an amber colored bottle because PI was photosensitive.
6. 10,000-20,000 cell events were acquired to get a statistically significant data.
7. The fluorescence intensity histograms were analyzed using the MODFIT software.

Propidium Iodide Buffer

| | | |
|---|---|---|
| Sodium citrate | 100 | mg |
| Propidium Iodide | 10 | mg |
| Rnase A | 2 | mg |
| Tritox ™ X-100 | 300 | μL |
| MilliQ ® water (autoclaved) | 100 | mL |

### (ii) In Vitro Angiogenesis Assay (Tube Formation)

Angiogenesis is the process of generating new capillary blood vessels. It is a fundamental component of a number of normal (reproduction and wound healing) and pathological processes (diabetic retinopathy, rheumatoid arthritis, tumor growth and metastasis). The CHEMICON In Vitro Angiogenesis Assay Kit was used as a convenient system for evaluation of tube formation by endothelial cells in a 96-well format. When cultured on ECMatrix™, a solid gel of basement proteins prepared from the Engelbreth Holm-Swarm (EHS) mouse tumor, these endothelial cells rapidly align and form hollow tube-like structures. Tube formation is a multi-step process involving cell adhesion, migration, differentiation and growth.

ECMatrix™ consists of laminin, collagen type IV, heparan sulfate proteoglycans, entactin and nidogen. It also contains various growth factors (TGF-beta, FGF) and proteolytic enzymes (plasminogen, tPA, MMPs) that occur normally in EHS tumors. It was optimized for maximal tube-formation.

### (i) Protocol:

96 well plate was coated with 1×ECM diluted with dilution buffer. Initial strength of ECM was 2×. ECM was incubated at 4° C. prior to coating.
ECM coated plate was incubated at 37° C. for 30 min and ECM to solidified.
HUVEC cells were seeded at 5000 cells/well and the plate incubated at 37° C. for 30 min.
The plate was treated with the inhibitors after 30 min.

A functional readout (tube formation/branching) was taken within 3-4 h of seeding and inhibitor treatment. Cells were visualized under a microscope connected to a CCD camera and snapshots were recorded.

All publications cited in this specification are incorporated herein by reference. While the invention has been described with reference to particular embodiments, it will be appreciated that modifications can be made without departing from the spirit of the invention. Such modifications are intended to fall within the scope of the appended claims.

LENGTHY TABLES
The patent application contains a lengthy table section. A copy of the table is available in electronic form from the USPTO web site (http://seqdata.uspto.gov/?pageRequest=docDetail&DocID=US20160222014A1). An electronic copy of the table will also be available from the USPTO upon request and payment of the fee set forth in 37 CFR 1.19(b)(3).

What is claimed is:

1. A compound of formula IA or IB having the following structure: 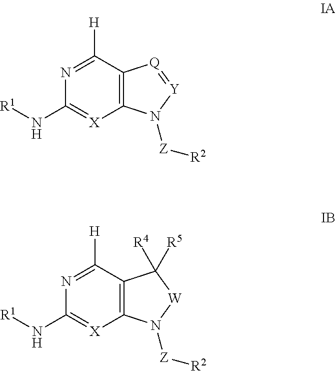
wherein:
X and Y are, independently, N or CH;
W is CH₂, —C═O, or NR¹³;
Z is absent, CH₂, —C═O or —SO₂, with the proviso that:
(i) Z is not —C═O or —SO₂ when W is C═O; and
(ii) Z is not absent, when Y is CH;
Q is N or CR³;
R¹ is optionally substituted C₆-C₁₀ aryl or optionally substituted C₂-C₁₀ heteroaryl;
R³ is H, C₁-C₆ alkyl, halogen, CN, or C₁-C₆ trifluoroalkyl;
R⁴ and R⁵ are, independently, H, F or optionally substituted C₁-C₆ alkyl; or
R⁴ and R⁵ are taken together to form a 3 to 6 membered cyclic ring having 0-1 heteroatom;
R² is optionally substituted C₆-C₁₀ aryl, optionally substituted heteroaryl, C₃-C₆ optionally substituted cycloalkyl or C₂-C₆ heterocyclyl;
R¹³ is H or optionally substituted alkyl;
or a pharmaceutically acceptable salt or prodrug thereof.

2. The compound according to claim 1, which is of formula IA:

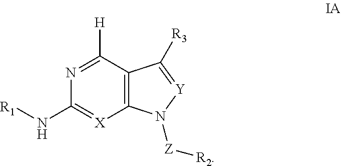

3. The compound according to claim 1, which is of formula IB:

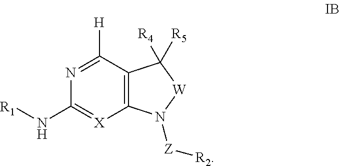

4. The compound according to claim 1, which is of formula IA-2 or IB-2:

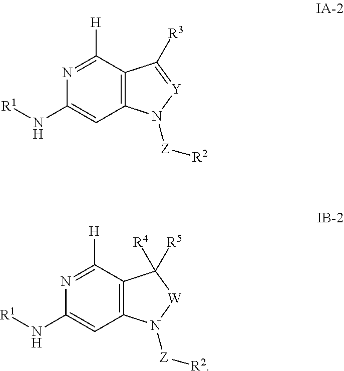

5. The compound according to claim 1, which is of formula IA-3 or IB-3:

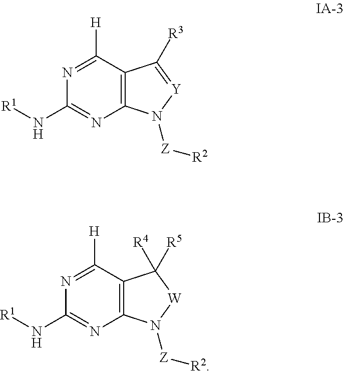

6. The compound according to claim 1, which is of formula IA-4 or IB-4:

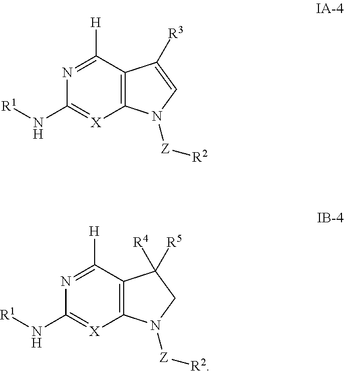

7. The compound according to claim 1, which is of formula IA-5:

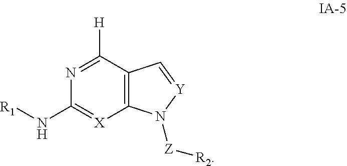

8. The compound according to claim 1, which is of formula IA-6:

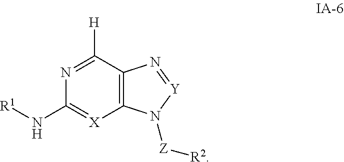

9. The compound according to claim 1 which is of formula IA-8:

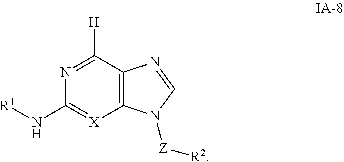

10. The compound according to claim 1, wherein R³ is H, CH₃, or F.

11. The compound according to claim 1, wherein R⁴ and R⁵ are, independently, H or CH₃.

12. The compound according to claim 1, wherein R⁴ and R⁵ are joined to form a cyclopropyl.

13. The compound according to claim 1, wherein R² is optionally substituted heteroaryl.

14. The compound according to claim 13, wherein R² is optionally substituted imidazole, pyridine, thiophene, quinoline, naphthalene, benzothiazole, or benzothiodiazole.

15. The compound according to claim 14, wherein R² is optionally substituted imidazole.

16. The compound according to claim 15, wherein R² is imidazole substituted with 1 or 2 C₁-C₆ alkyl.

17. The compound according to claim 16, wherein R² is imidazole substituted with 1 or 2 CH₃ groups.

18. The compound according to claim 14, wherein R² is optionally substituted pyridine.

19. The compound according to claim 18, wherein R² is pyridine substituted with 1 or more C₁-C₆ alkoxy, N(C₁-C₆ alkyl)OSO₂(C₁-C₆ alkyl), N(C₁-C₆ alkyl)(SO₂(C₁-C₆ alkyl), or N(C₁-C₆ alkyl)SO₂(C₃-C₈ cycloalkyl).

20. The compound according to claim 19, wherein R² contains a SO₂ group in the backbone of ring.

21. The compound according to claim 19 or 20, wherein R² is 1-N(CH₃)(OSO₂CH₃)-pyridin-2-yl, 1-N(CH₃)SO₂CH₃-pyridin-2-yl, 2-N(CH₃)SO₂CH₃-pyridin-3-yl, pyridine-2-yl, pyridine-3-yl, 2-OCH₃-pyridin-4-yl, 2-N(CH₃)SO₂-cyclopropyl-pyridin-3-yl, or dioxidoisothiazolidin-2-yl.

22. The compound according to claim 14, wherein R² is optionally substituted quinoline.

23. The compound according to claim 22, wherein R² is quinoline substituted with 1 or more C₁-C₆ alkyl.

24. The compound according to claim 22, wherein said quinoline contains a C(O) in the backbone of the ring.

25. The compound according to claim 22, wherein R² is quinolone, 4-CH₃-quinolin-8-yl, 2-CH₃-quinolin-8-yl, 6-CH₃-quinolin-8-yl, or 8-isoquinoline.

26. The compound according to claim 14, wherein R² is thiophene.

27. The compound according to claim 1, wherein R² is optionally substituted aryl.

28. The compound according to claim 27, wherein R² is optionally substituted phenyl.

29. The compound according to claim 27, wherein R² is phenyl substituted with 1 or more of halogen, C₁-C₆ alkoxy, C₁-C₆ trifluoroalkyl, C₁-C₆ alkyl, CN, NH₂, C₁-C₆ trifluoroalkoxy, SO₂N(C₁-C₆ alkyl)₂, SO₂NH(C₁-C₆ alkyl), SO₂(C₁-C₆ alkyl), N(C₁-C₆ alkyl)SO₂(C₁-C₆ alkyl), N(C₃-C₈ cycloalkyl)SO₂(C₁-C₆ alkyl), NHC(O)(C₁-C₆ alkyl), N(C₁-C₆hydroxyalkyl)SO₂(C₁-C₆ alkyl), N(alkylamino)SO₂(C₁-C₆ alkyl), N(C₁-C₆ alkoxy)SO₂(C₁-C₆ alkyl), —(C₁-C₆ alkyl)-C(O)NH(C₁-C₆ alkyl), or N(C₁-C₆ alkyl-morpholine)SO₂(C₁-C₆ alkyl).

30. The compound according to claim 27, wherein R² is phenyl, 2,3-di-Cl-phenyl, 2,5-di-Cl-phenyl, 2,5-di-OCH₃-phenyl, 2-5-di-Cl-phenyl, 2-CF₃-phenyl, 2-CH₃-phenyl, 2-Cl-5-CH₃-phenyl, 3-Cl-phenyl, 3-CN-phenyl, 2-Cl-phenyl, 2-F-phenyl, 2-OCF₃-phenyl, 2-OCH₃-phenyl, 2-NH₂-phenyl, 4-tolyl, 3-OCH₃-phenyl, 4-OCF₃-phenyl, 3-OCF₃-phenyl, 2-OCHF₂-phenyl, 2-SO₂N(CH₃)₂-phenyl, 2-NHSO₂CH₃-phenyl, 2-SO₂—NHCH₃-phenyl, 3-SO₂—NHCH₃-phenyl, 3-SO₂(CH₃)-phenyl, 2-N(CH₃)SO₂CH₃-phenyl,2-N(CH₃)SO₂CH₃-3-OCH₃-phenyl, 2-N(cyclopropyl)SO₂CH₃-phenyl, 3-N(CH₃)SO₂(CH₃)-phenyl, 2-NH(CH₃)—SO₂CH₃-phenyl, 4-NHC(O)CH₃-phenyl, 2-N(CH₂CH₂OH)SO₂CH₃-phenyl, 2-N(CH₂CH₂NH₂)SO₂CH₃-phenyl, 2-N(CH₂CH₂OCH₃)SO₂CH₃-phenyl, 2-CH₂—C(O)NHCH₃-phenyl, 2-N(CH₂CH₂-morpholine)SO₂CH₃-phenyl.

31. The compound according to claim 1, wherein R² is optionally substituted C₃-C₈ cycloalkyl.

32. The compound according to claim 31, wherein R² is cyclopentyl or cyclopropyl.

33. The compound according to claim 1, wherein:
R² is C₆-C₁₀ aryl or heteroaryl substituted with one or more R¹²;
R¹² is H, optionally substituted C₁-C₆ alkyl, optionally substituted C₂-C₆ alkenyl, optionally substituted C₂-C₆ alkynyl, alkoxy, —S(O)ₙ—C₁-C₆ alkyl, —O(CH₂)ₐNR⁸R⁹, —O(CH₂)ₐOH, —O(CH₂)ₐO—C₁-C₆ alkyl, CN, aryl, heteroaryl, optionally substituted monocyclic cycloalkyl, optionally substituted bicyclic cycloalkyl, optionally substituted monocyclic heterocyclyl, optionally substituted bicyclic heterocyclyl, (aryl)alkyl, COOH, NH₂, NR⁸R⁹, —C(O)NH₂, —C(O)NR¹⁰R¹¹, —SO₂NH₂, —SO₂NR¹⁰R¹¹, aminoalkyl, (alkyl)amido, (alkyl)amino, arylalkyl, alkylcarboxyl, (alkyl)carboxyamido, heterocyclyl(alkyl), heteroaryl(alkyl) (aryl)oxy, (heteroaryl)oxy, halogen, hydroxyalkyl, perfluoroalkyl, monofluoroalkyloxy, difluoroalkyloxy or perfluoroalkyloxy;
a is 2-6;
n is 0-2;
R⁸ and R⁹ are, independently, H, optionally substituted C₁-C₆ alkyl, —C(O)C₁-C₆ alkyl, —C(O)C₃-C₆ cycloalkyl, —C(O)-heterocyclyl, —CONR¹⁰R¹¹, —SO₂-optionally substituted C₁-C₆ alkyl, —SO₂ (alkyl)(C₁-C₆ hydroxyalkyl), —SO₂(alkyl)(C₁-C₆ alkoxyalkyl), —SO₂(alkyl)(C₁-C₆ alkylamino), —SO₂-aryl, —SO₂-heteroaryl, —SO₂—C₃-C₇ cycloalkyl, —SO₂—C₄-C₆ heterocyclyl, —C(O)O—C₁-C₆ alkyl, —C(O)O—C₃-C₆ cycloalkyl, —C(O)O—C₄-C₆ heterocyclyl, C₁-C₆ alkylcarbonyl, C₁-C₆ hydroxyalkyl; or
R⁸ and R⁹ are taken together to form a 3 to 7 membered saturated or partially saturated ring optionally having 1 or more heteroatom and wherein said 3 to 7 membered ring is optionally substituted with optionally substituted C₁-C₆ alkyl, hydroxyl, halogen, C₁-C₆ alkoxy, NH₂, or NR⁸R⁹, or one or more —CH₂— of the ring are replaced by C═O;
R¹⁰ and R¹¹ are, independently, H, C₁-C₆ alkyl, aryl, heteroaryl, mono cycloalkyl, bicyclic cycloalkyl, mono heterocyclyl, or bicyclic heterocyclyl; or
R¹⁰ and R¹¹ are taken together to form a 3 to 7 membered saturated or partially saturated ring optionally having 1 or more heteroatom and wherein said 3 to 7 membered ring is optionally substituted with C₁-C₆ alkyl or one or more —CH₂— of the ring are replaced by C═O, (aryl)alkyl, hydroxyalkyl or perfluoroalkyl.

34. The compound according to claim 33, wherein R¹² is an aminoalkyl.

35. The compound according to claim 34, wherein R¹² is —(CH₂)ₐNH₂ or —(CH₂)ₐNR⁸R⁹.

36. The compound according to claim 33, wherein R¹² is an alkylcarboxyl.

37. The compound according to claim 36, wherein R¹² is —OC(O)C₁-C₆ alkyl.

38. The compound according to claim 1, wherein R¹ is optionally substituted C₆-C₁₀ aryl.

39. The compound according to claim 38, wherein R¹ is optionally substituted phenyl.

40. The compound according to claim 39, wherein R¹ is phenyl substituted with one or more C₁-C₆ alkoxy, C₁-C₆ alkyl, O—C₃-C₈ cycloalkyl, halogen, C₁-C₆ hydroxyalkyl, CN, NHC(O)C₁-C₆ alkyl, NHC(O)C(C₁-C₆hydroxalkyl)(C₁-C₆ alkyl), —OC₁-C₆ alkyl-N(C₁-C₆ alkyl)₂, C₁-C₆ alkylC(O)NH₂-heterocyclyl, C(O)NH₂, C₁-C₆ trifluoroalkyl, C(O)NH(C₁-C₆ alkyl)-O—C₁-C₆ alkyl, N(C₁-C₆ alkyl)₂, NHC(O)C₁-C₆hydroxyalkyl, NHC(O)(C₃-C₈ cycloalkyl), S(C₁-C₆ alkyl), SO₂NH₂, SO₂(C₁-C₆ alkyl), SO₂—C₁-C₆ trifluoroalkoxy, O—C₁-C₆ alkyl(heterocyclyl), —CH₂—(C₁-C₆ hydroxyalkyl-heterocyclyl), C₁-C₆ alkyl-CN, NH(C₁-C₆ hydroxyalkyl), —(C₁-C₆ alkyl)N(C₁-C₆ alkyl)₂, C(O)(heterocyclyl), oxo-(heterocyclyl), (heterocyclyl), dioxo-(heterocyclyl), N(C₁-C₆ alkyl)₂(heterocyclyl), OC(O)C₁-C₆ alkyl(heterocyclyl), C₁-C₆ hydroxyalkyl(heterocyclyl), OH substituted heterocyclyl, NH(C₁-C₆ alkyl)-(heterocyclyl), C₁-C₆ alkyl-NH₂-heterocyclyl, heterocyclyl substituted heterocyclyl, heteroaryl substituted heterocyclyl, NH₂ substituted heterocyclyl, halogen substituted heterocyclyl, —O-(heterocyclyl), NH(C₁-C₆ alkyl)SO₂(C₁-C₆ alkyl)-phenyl, di-(C₁-C₆ alkyl)(heterocyclyl), C₁-C₆ alkyl(heterocyclyl), C(O)(C₃-C₈ cycloalkyl)heterocyclyl, C(O)(C₁-C₆ alkyl)(heterocyclyl), 1-C₁-C₆ alkyl-O—C(O)C₁-C₆ alkyl-(heterocyclyl), C₁-C₆ alkoxy(heterocyclyl), 1-C₁-C₆ hydroxyalkyl(heterocyclyl), C₃-C₈ cycloalkyl-heterocyclyl, heteroaryl, or oxo-heteroaryl.

41. The compound according to claim 39, wherein R¹ is phenyl optionally substituted with C₁-C₆ alkoxy, C₁-C₆ alkyl, O—C₃-C₈ cycloalkyl, halogen, C₁-C₆ hydroxyalkyl, CN, NHC(O)C₁-C₆ alkyl, NHC(O)C(C₁-C₆ hydroxalkyl)(C₁-C₆ alkyl), OC₁-C₆ alkyl-N(C₁-C₆ alkyl)₂, C(O)NH₂, C₁-C₆ trifluoroalkyl, C(O)NH—C₁-C₆ alkyl-O—C₁-C₆ alkyl, N(C₁-C₆ alkyl)₂, NHC(O)C₁-C₆ hydroxyalkyl, NHC(O)(C₃-C₈ cycloalkyl), S(C₁-C₆ alkyl), SO₂NH₂, SO₂(C₁-C₆ alkyl), SO₂—C₁-C₆ trifluoroalkoxy, O—C₁-C₆ alkyl-morpholine, O—C₁-C₆ alkyl-piperazine, —CH₂—(C₁-C₆ hydroxyalkyl-pyrrolidine), —C₁-C₆ alkyl-pyrrolidine, C₁-C₆ alkyl-CN, NH(C₁-C₆ hydroxyalkyl), NH(C₁-C₆ alkyl), —(C₁-C₆ alkyl)N(C₁-C₆ alkyl)₂, C(O)morpholine, oxo-morpholine, morpholine, dioxo-thiomorpholine, thiomorpholine, N(C₁-C₆ alkyl)₂-piperidine, OC(O)C₁-C₆ alkyl-piperidine, C₁-C₆ hydroxyalkyl-piperidine, 4-OH-piperidine, NH(C₁-C₆ alkyl)-piperidine, C₁-C₆ alkyl-NH₂-piperidine, morpholine-piperidine, piperazine-piperidine, OH-piperidine, NH₂-piperidine, C₁-C₆ alkoxy-piperidine, piperidine, tetrazole, N(C₁-C₆ alkyl)₂-pyrrolidine, N(C₁-C₆ alkyl)₂-azetidine, azetidin-3-yloxy, NH(C₁-C₆ alkyl)SO₂ (C₁-C₆ alkyl)-phenyl, pyridine, oxo-pyridine, tetrahydropyridine, piperazine, (C₁-C₆ alkyl)₂piperazine, C₁-C₆ alkyl-piperazine, C(O)(C₃-C₈ cycloalkyl)-piperazine, C(O)(C₁-C₆ alkyl)-piperazine, 1-C₁-C₆ alkyl-O—C(O)C₁-C₆ alkyl-piperazine, C₁-C₆ alkoxy-piperazine, 1-C₁-C₆ hydroxyalkyl-piperazine, oxo-piperazine, C₃-C₈ cycloalkyl-piperazine, C(O)-piperazine, C₁-C₆ alkyl-diazepane, diazepane, 2,5-diaza-bicyclo[2.2.1]hept-2-yl optionally substituted with C₁-C₆ alkyl and C(O)(C₁-C₆ alkyl), hexahydro-pyrrolo[1,2-a]pyrazine optionally containing an oxo group in the backbone of the ring

42. The compound according to claim 39, wherein R¹ is 1-OCH₃-3-CH₃-phenyl, 2-(O-cyclobutyl)-4-piperazin-4-yl-phenyl, 2,6-di-F-phenyl, 2-CH₂OH-4-piperazin-4-yl-phenyl, 2-CH₃-4-(1-CH₂CH₂OH-piperazin-4-yl)-phenyl, 2-CH₃-phenyl, 2-CH₃-piperazin-4-yl-phenyl, 2-CN-phenyl, 2-F-4-(2-CH₃-piperazin-4-yl)-phenyl, 2-F-4-piperazin-4-yl-phenyl, 2-F-5-NHC(O)CH₃-phenyl, 1-CH₂C(O)NH₂-piperidin-4-yl-phenyl, 4,4-di-F-piperidin-1-yl-phenyl,-2-F-phenyl, 2-NHC(O)CH₃-phenyl, 2-OCH₃-3-F-4-C(O)morpholine-phenyl, 2-OCH₃-3-F-4-piperazin-4-yl-phenyl, 2-OCH₃-4-(1-CH₂CH₂OH-piperazin-4-yl)-phenyl, 2-OCH₃-4-(1-CH₃-piperazin-4-yl)-phenyl, 2-OCH₃-4-(4-N(CH₃)₂-piperidin-1-yl)-phenyl, 2-OCH₃-4-(4-OC(O)CH₃-piperidin-1-yl)-phenyl, 2-OCH₃-4-(piperidin-4-ol)-phenyl, 2-OCH₃-4-C(O)-morpholine-phenyl, 2-OCH₃-4-morpholine-phenyl, 2-OCH₃-4-piperazin-4-yl-phenyl, 2-OCH₃-phenyl, 3-NHC(O)C(CH₃)₂OH-phenyl, 3-(tetrazol-5-yl)-phenyl, 3-OCH₂CH₂N(CH₃)₂)-phenyl, 3,4,5-tri-OCH₃-phenyl, 3-C(O)NH₂-phenyl, 3-C(O)NHCH₃-phenyl, 3-CF₃-phenyl, 3-CH₂OCH₃-phenyl, 3-CH₂OH-4-(2-CH₃-piperazinyl)-phenyl, 3-CH₂OH-4-(piperazin-4-yl)-phenyl, 3-CH₃-(1-CH₃-piperazin-4-yl)-phenyl, 3-CH₃-4-(3-CH₃-piperazinyl)-phenyl, 3-CH₃-4-(4-N(CH₃)₂-piperidin-1-yl)-phenyl, 3-CH₃-4-morpholine-phenyl, 3-CH₃-4-piperazin-4-yl-phenyl, 3-CN-phenyl, 3-F-4-(2-CH₃-piperazinyl)-phenyl, 3-F-4-(3-CH₃-piperazinyl)-phenyl, 3-F-4-(3-N(CH₃)₂-pyrrolidin-1-yl)-phenyl, 3-F-4-(4-N(CH₃)₂-piperidin-1-yl)-phenyl, 3-F-4-(C(O)NHCH₂C(CH₃)₂OCH₃)-phenyl, 3-F-4-(C(O)NHCH₂C(CH₃)OH)-phenyl, 3-F-4-(N(CH₃)₂-azetidin-1-yl)-phenyl, 3-F-4-C(O)-morpholine-phenyl, 3-F-4-piperazin-4-yl-phenyl, 3-F-phenyl, 3-morpholine-phenyl, 3-N(CH₃)₂-phenyl, 3-NHC(O)CH(CH₃)OH-phenyl, 3-NHC(O)CH₃-phenyl, 3-OCH₃-4-(1-CH₂CH₂OH-piperazin-4-yl)-phenyl, 3-OCH₃-4-(3-CH₃-piperazin-4-yl)-phenyl, 3-OCH₃-4-(4-piperidin-4-ol)-phenyl, 3-OCH₃-4-morpholine-phenyl, 3-OCH₃-4-NHC(O)(cyclobutyl)-phenyl, 3-OCH₃-4-NHC(O)(cyclopropyl)-phenyl, 3-OCH₃-4-piperazin-4-yl-phenyl, 3-OCH₃-phenyl, 4-(3-OH-piperidin-1-yl)-phenyl, 3-piperazine-phenyl, 3-piperazinyl-5-(3-N(CH₃)SO₂CH₃-phenyl)-phenyl, 3-piperazinyl-5-(pyridin-3-yl)-phenyl, 3-SCH₃-phenyl, 3-SO₂NH₂-phenyl, 4-(1,1-dioxo-thiomorpholin-4-yl)-phenyl, 4-(1,2-di-CH₃-piperazin-4-yl)-phenyl, 4-(1-C(O)(cyclopropyl))piperazin-1-yl-phenyl, 4-(1-C(O)CH₃)-piperazin-4-yl-phenyl, 4-(1-C(O)CH₃-2-CH₃-piperazin-4-yl)-phenyl, 4-(1-CH₂CH₂O—C(O)CH₃-piperazin-4-yl)-phenyl, 4-(1-CH₂CH₂OCH₃-piperazin-4-yl)-phenyl, 4-(1-CH₂CH₂OH-piperazin-4-yl)-phenyl, 4-(1-CH₂CH₃-piperazin-4-yl)-phenyl, 4-(1-CH₃-piperazin-4-yl)-phenyl, 4-(2,5-di-CH₃-piperazin-4-yl)-phenyl, 4-(2-CH₃-piperazin-4-yl)-phenyl, 4-(2-oxo-morpholine)-phenyl, 4-(2-oxo-piperazinyl-phenyl, 4-(2-oxo-pyridin-1-yl)-phenyl, 4-(3,3-di-CH₃-piperazinyl)-phenyl, 4-(3,5-di-CH₃-piperazin-4-yl)-phenyl, 4-(3-CH₂CH₃-piperazinyl-1-yl)-phenyl, 4-(3-CH₂NH₂-piperidin-1-yl)-phenyl, 4-(3-CH₂OH-piperidin-1-yl)-phenyl, 4-(3-CH₃-piperazin-1-yl)-phenyl, 4-(2-oxo-3,3-di-CH₃-piperazin-1-yl)-phenyl, di-(C₁-C₆ alkyl)-oxo-piperazine, 4-(3-N(CH₃)₂-azetidin-1-yl)-phenyl, 4-(3-N(CH₃)₂-piperidin-1-yl)-phenyl, 4-(4-(azetidin-3-yloxy)-phenyl, 4-(4-(morpholinyl)-piperidin-1-yl)-phenyl, 4-(4-(piperazin-1-yl)-piperidin-1-yl)-phenyl, 4-(4-CH₂CH(CH₃)OH-piperazin-1-yl)-phenyl, 4-(4-CH₂CH(CH₃)-piperazin-1-yl)-phenyl, 4-(4-CH₂OH-piperidin-1-yl)-phenyl, 4-(4-CH₃-4-OH-piperidin-1-yl)-phenyl, 4-(4-CH₃-piperazin-1-yl)-phenyl, 4-(4-cyclopropyl-piperazinyl-1-yl)-phenyl, 4-(4-methyl-[1,4]diazepan-1-yl)-phenyl, 4-(4-N(CH₃)₂-piperidin-1-yl)-phenyl, 4-(4-N(CH₃)CH₂CH₂OH-piperidin-1-yl)-phenyl, 4-(4-NH₂-piperidin-1-yl)-phenyl, 4-(4-NHCH₃-piperidin-1-yl)-phenyl, 4-(4-OC(O)CH₃-piperidin-1-yl)-phenyl, 4-(4-piperidin-1-OCH₃)-phenyl, 4-(NHC(O)C(CH₃)₂OH)-phenyl, 4-(OCH₂CH₂-morpholine)-phenyl, 4-(OCH₂CH₂-piperazin-4-yl)-phenyl, 4-(O-piperidin-4-yl)-phenyl, 4-(piperazin-2-yl)-phenyl, 4-(piperazine-2-one)-phenyl, 4-(piperazinyl-1-yl)-phenyl, 4-(piperidin-4-ol)-phenyl, 4-[1,4]diazepan-1-yl-phenyl, 4-C(O)-piperazin-4-yl-phenyl, 4-CH₂-(2-CH₂OH)-pyrrolidin-1-yl-phenyl, 4-CH₂-piperazin-4-yl-phenyl, 4-CH₂-pyrrolidin-1-yl-phenyl, 4-CH₂CN-phenyl, 4-CN-phenyl, 4-F-phenyl, 4-morpholine-phenyl, 4-N(CH₃)₂-phenyl, 4-NHCH₂CH₂OH-phenyl, 4-NHC(O)(cyclopropyl)-phenyl, 4-NHC(O)CH(CH₃)₂-phenyl, 4-NHC(O)CH₃-phenyl, 4-NHCH₃-phenyl, 4-O-(4-NHCH₃-piperidin-1-yl)-phenyl, 4-OCH₃-phenyl, 4-piperazin-4-yl-phenyl, 4-piperidin-4-yl-phenyl, 4-SO₂CH₃-phenyl, 4-SO₂—CHF₂-phenyl, 4-tetrahydropyridin-4-yl-phenyl, 4-thiomorpholino-phenyl, CH₂N(CH₃)₂-phenyl, phenyl, NHC(O)CH₃-phenyl, OCH₂CH₂N(CH₃)₂-phenyl, 3-OCH₃-4-(5-methyl-(2,5-diaza-bicyclo[2.2.1]hept-2-yl))-phenyl, 4-(2,5-diaza-2-C(O)CH₃-bicyclo[2.2.1]hept-2-yl)-phenyl, 4-(2,5-diaza-bicyclo[2.2.1]hept-2-yl)-phenyl, 4-(6-oxohexahydropyrrolo[1,2-a]pyrazin-2-yl)-phenyl, 4-(hexahydro-pyrrolo[1,2-a]pyrazin-2-yl)-phenyl

43. The compound according to claim 1, wherein R¹ is optionally substituted C₂-C₁₀ heteroaryl.

44. The compound according to claim 43, wherein R¹ is optionally substituted pyrazole, quinoline, pyridine, pyrimidine, dihydrobenzooxazole, benzooxazole, benzoimidazole, dihydroisobenzofuran, isobenzofuran, dihydrobenzooxazine, dibenzooxazine, benzotriazole, benzothiazole, benzothiophene, indazole, hexahydropyrazinoindole, indoline, or tetrahydroquinolinyl.

45. The compound according to claim 43, wherein R¹ contains an oxo group in the backbone of said heteroaryl.

46. The compound according to claim 43, wherein R¹ is substituted with one or more C₁-C₆ alkyl, optionally substituted heterocyclyl, CN, NHC(O)(C₁-C₆ alkyl), optionally substituted heteroaryl, —(C₁-C₆ alkyl)N(C₁-C₆ alkyl)₂, or C₁-C₆ hydroxyalkyl.

47. The compound according to claim 43, wherein R¹ is substituted with one or more C₁-C₆ alkyl, piperidine, CN, NHC(O)(C₁-C₆ alkyl), piperazin-2-one, morpholine, C₁-C₆ alkyl substituted pyridine, —(C₁-C₆ alkyl)N(C₁-C₆ alkyl)₂, or C₁-C₆ hydroxyalkyl.

48. The compound according to claim 43, wherein R¹ is 1-(piperidin-4-yl)-pyrazol-4-yl, 1-CH₃-pyrazol-4-yl, 1-methyl-2-oxo-1,2,3,4-tetrahydroquinolin-6-yl, 2-dihydroquinoline-2-one, 2-oxo-1,2,3,4-tetrahydro-quinolin-6-yl, 6-quinolinyl, 2-CN-pyridin-5-yl, 2-NHC(O)CH₃-pyridin-6-yl, 3-(piperazine-2-one)-pyridin-6-yl, 3-morpholine-pyridin-6-yl, 3-NHC(O)CH₃-pyridin-6-yl, 3-pyridinyl, 4-(3-CH₃-piperazinyl)-pyridin-3-yl, 4-pyridinyl, pyrimidinyl, 2,3-dihydro-benzooxazol-2-one, 5-benzoimidazolyl, 5-benzimidazol-2-one, 2-oxo-2,3-dihydro-benzoimidazol-5-yl, 3-oxo-1,3-dihydroisobenzofuran-5-yl, 3-oxo-3,4-dihydro-benzooxazin-7-yl, 3-oxo-3,4-dihydro-benzoxazin-6-yl, 3,4-dihydro-benzooxazin-6-yl, 1,4-benzoxazin-7-yl, 5-benzotriazolyl, 6-benzothiazolyl, 6-benzothiophene-yl, 6-indazolyl, 1,2,3,4,10,10a-hexahydro-pyrazino[1,2-a]indol-8-yl, 1,3-dioxo-2,3-dihydro-isoindol-5-yl, 1-CH₂CH₂N(CH₃)₂-2-oxo-indolin-5-yl, 1-CH₂CH₂OH-2-oxo-indol-5-yl, 1-methyl-2-oxo-2-3-dihydro-indol-5-yl, 3-(1,2,3,6-tetrahydropyridin-4-yl)-indol-5-yl, 3-(tetrahydropyridin-4-yl)-indol-5-yl, 5-indolin-2-one, or 5-indolinyl.

49. The compound according to claim 1, wherein:
R¹ is heteroaryl substituted with one or more R⁶;
R⁶ is H, optionally substituted C₁-C₆ alkyl, alkoxy, —O(CH₂)ₐNR⁸R⁹, —O(CH₂)ₐOH, —O(CH₂)ₐO—C₁-C₆ alkyl, CN, optionally substituted aryl, optionally substituted heteroaryl, monocyclic or bicyclic optionally substituted cycloalkyl, monocyclic or bicyclic optionally substituted heterocyclyl, monocyclic or —O-bicyclic optionally substituted heterocyclyl, (aryl)alkyl, C(O)OH, NH₂, NR⁸R⁹, —C(O)NH₂, —C(O)NR¹⁰R¹¹, —S-(optionally substituted C₁-C₆ alkyl), —SO₂—C₁-C₆ alkyl, —SO₂—NH(C₁-C₆ alkyl), —SO₂—N(C₁-C₆ alkyl)₂, —NHC(O)C₁-C₆-alkyl, —NHC(O)C₁-C₆-alkyl substituted with OH, —SO₂NH₂, —SO₂NR¹⁰R¹¹, aminoalkyl, (alkyl)amido, (alkyl)amino, arylalkyl, alkylcarboxyl, (alkyl)carboxyamido, heterocyclyl(alkyl), heteroaryl(alkyl), (aryl)oxy, (heteroaryl)oxy, halogen, hydroxyalkyl, —S(O)ₙ-perfluoroalkyl, perfluoroalkyl, monofluoroalkyloxy, difluoroalkyloxy or perfluoroalkyloxy;
a is 2-6;
n is 0-2;
R⁸ and R⁹ are, independently, H, optionally substituted C₁-C₆ alkyl, —C(O)C₁-C₆ alkyl, —C(O)C₃-C₆ cycloalkyl, —CO-heterocyclyl, —CONR¹⁰R¹¹, —SO₂-optionally substituted C₁-C₆ alkyl, —SO₂ (alkyl) (C₁-C₆ hydroxyalkyl), —SO₂ (alkyl)(C₁-C₆ alkoxyalkyl), —SO₂(alkyl)(C₁-C₆ alkylamino), —SO₂-aryl, —SO₂-heteroaryl, —SO₂—C₃-C₇ cycloalkyl, —SO₂—C₄-C₆ heterocyclyl, —C(O)O—C₁-C₆ alkyl, —C(O)O—C₃-C₆ cycloalkyl, —C(O)O—C₄-C₆ heterocyclyl, C₁-C₆ alkylcarbonyl, C₁-C₆ hydroxyalkyl; or
R⁸ and R⁹ are taken together to form a 3 to 7 membered saturated or partially saturated ring optionally having 1 or more heteroatom and wherein said 3 to 7 membered ring is optionally substituted with optionally substituted C₁-C₆ alkyl, hydroxyl, halogen, C₁-C₆ alkoxy, NH₂, or NR⁸R⁹, or one or more —CH₂— of the ring are replaced by C═O;
R¹⁰ and R¹¹ are, independently, H, C₁-C₆ alkyl, aryl, heteroaryl, mono cycloalkyl, bicyclic cycloalkyl, mono heterocyclyl, or bicyclic heterocyclyl; or
R¹⁰ and R¹¹ are taken together to form a 3 to 7 membered saturated or partially saturated ring optionally having 1 or more heteroatom and wherein said 3 to 7 membered ring is optionally substituted with C₁-C₆ alkyl or one or more —CH₂— of the ring are replaced by C═O, (aryl)alkyl, hydroxyalkyl or perfluoroalkyl.

50. The compound according to claim 49, wherein R⁶ is an aminoalkyl.

51. The compound according to claim 50, wherein R⁶ is —(CH₂)ₐNH₂ or —(CH₂)ₐNR⁸R⁹.

52. The compound according to claim 51, wherein R⁶ is an alkylcarboxyl.

53. The compound according to claim 52, wherein R⁶ is —OC(O)C₁-C₆ alkyl.

54. The compound according to claim 1, wherein R⁴ and R⁵ are taken together to form a 3-8 membered cyclic system having it its backbone one O, S(O)ₙ or NR⁷, wherein R⁷ is H, optionally substituted C₁-C₆ alkyl, —SO₂-(optionally substituted C₁-C₆ alkyl, (alkoxy)carbonyl-, (alkyl)amido-, —C(O)C₁-C₆ alkyl or alkoxy alkyl.

55. The compound according to claim 1, which is selected from the group consisting of:
6-((7-(3-(methylsulfonyl)benzyl)-7H-pyrrolo[2,3-d]pyrimidin-2-yl)amino)-3,4-dihydroquinolin-2(1H)-one;
5-((7-(3-(methylsulfonyl)benzyl)-7H-pyrrolo[2,3-d]pyrimidin-2-yl)amino)indolin-2-one;
6-((7-(pyridin-3-ylmethyl)-7H-pyrrolo[2,3-d]pyrimidin-2-yl)amino)-3,4-dihydroquinolin-2(1H)-one;
5-((7-(pyridin-3-ylmethyl)-7H-pyrrolo[2,3-d]pyrimidin-2-yl)amino)indolin-2-one;
N-methyl-N-(3-((2-((2-oxo-1,2,3,4-tetrahydroquinolin-6-yl)amino)-7H-pyrrolo[2,3-d]pyrimidin-7-yl)methyl)pyridin-2-yl)methanesulfonamide;
1-(pyridin-3-ylmethyl)-N-(3,4,5-trimethoxyphenyl)-1H-pyrrolo[3,2-c]pyridin-6-amine;
6-((1-(pyridin-3-ylmethyl)-1H-pyrrolo[3,2-c]pyridin-6-yl)amino)-3,4-dihydroquinolin-2(1H)-one;
N-(4-((difluoromethyl)sulfonyl)phenyl)-1-(pyridin-3-ylmethyl)-1H-pyrrolo[3,2-c]pyridin-6-amine;
7-(2-(difluoromethoxy)benzyl)-N-(4-((difluoromethyl)sulfonyl)phenyl)-7H-pyrrolo[2,3-d]pyrimidin-2-amine;
N-(3-(methylthio)phenyl)-1-(pyridin-3-ylmethyl)-1H-pyrazolo[3,4-d]pyrimidin-6-amine;
5-((1-(pyridin-3-ylmethyl)-1H-pyrazolo[3,4-d]pyrimidin-6-yl)amino)indolin-2-one;
7-(2-(difluoromethoxy)benzyl)-N-(3-(methylthio)phenyl)-7H-pyrrolo[2,3-]pyrimidin-2-amine;
N-(3-(methylthio)phenyl)-1-(pyridin-3-ylmethyl)-1H-pyrrolo[3,2-c]pyridin-6-amine;
6-((1-(pyridin-3-ylmethyl)-1H-pyrazolo[3,4-d]pyrimidin-6-yl)amino)-3,4-dihydroquinolin-2(1H)-one;
N-(1-(pyridin-3-ylmethyl)-1H-pyrrolo[3,2-c]pyridin-6-yl)benzo[d]thiazol-6-amine;
1-(pyridin-3-ylmethyl)-N-(3-(trifluoromethyl)phenyl)-1H-pyrrolo[3,2-c]pyridin-6-amine;
N-(4-((difluoromethyl)sulfonyl)phenyl)-7-(pyridin-3-ylmethyl)-7H-pyrrolo[2,3-d]pyrimidin-2-amine;
6-((7-(2-(difluoromethoxy)benzyl)-7H-pyrrolo[2,3-d]pyrimidin-2-yl)amino)-3,4-dihydroquinolin-2(1H)-one;
1-(2-(difluoromethoxy)benzyl)-N-(4-((difluoromethyl)sulfonyl)phenyl)-1H-pyrrolo[3,2-c]pyridin-6-amine;
N-(1-(2-(difluoromethoxy)benzyl)-1H-pyrrolo[3,2-c]pyridin-6-yl)benzo[d]thiazol-6-amine;
6-((1-(2-(difluoromethoxy)benzyl)-1H-pyrazolo[3,4-d]pyrimidin-6-yl)amino)-3,4-dihydroquinolin-2(1H)-one;
N-(3-((6-((6-cyanopyridin-3-yl)amino)-1H-pyrrolo[3,2-c]pyridin-1-yl)methyl)pyridin-2-yl)-N-methylmethanesulfonamide;
N-(3-((2-((6-cyanopyridin-3-yl)amino)-7H-pyrrolo[2,3-d]pyrimidin-7-yl)methyl)pyridin-2-yl)-N-methylmethanesulfonamide;
6-((1-(2-(difluoromethoxy)benzyl)-1H-pyrrolo[3,2-c]pyridin-6-yl)amino)-3,4-dihydroquinolin-2(1H)-one;
N-(3-((6-((4-((difluoromethyl)sulfonyl)phenyl)amino)-1H-pyrrolo[3,2-c]pyridin-1-yl)methyl)pyridin-2-yl)-N-methylmethanesulfonamide;
N-(3-((1-((2-(N-methylmethylsulfonamido)pyridin-3-yl)methyl)-1H-pyrrolo[3,2-c]pyridin-6-yl)amino)phenyl)acetamide;
3-((1-((2-(N-methylmethylsulfonamido)pyridin-3-yl)methyl)-1H-pyrrolo[3,2-c]pyridin-6-yl)amino)benzamide;
N-(3-((7-((2-(N-methylmethylsulfonamido)pyridin-3-yl)methyl)-7H-pyrrolo[2,3-d]pyrimidin-2-yl)amino)phenyl)acetamide;
3-((7-((2-(N-methylmethylsulfonamido)pyridin-3-yl)methyl)-7H-pyrrolo[2,3-d]pyrimidin-2-yl)amino)benzamide;
N-(3((6-(benzo[d]thiazol-6-ylamino)-1H-pyrrolo[3,2-c]pyridin-1-yl)methyl)pyridin-2-yl)-N-methylmethanesulfonamide;
N-methyl-N-(3-((6-((2-oxo-1,2,3,4-tetrahydroquinolin-6-yl)amino)-1H-pyrrolo[3,2-c]pyridin-1-yl)methyl)pyridin-2-yl)methanesulfonamide;
N-(4-((1-((2-(N-methylmethylsulfonamido)pyridin-3-yl)methyl)-1H-pyrrolo[3,2-c]pyridin-6-yl)amino)phenyl)acetamide;
6-((1-(pyridin-2-yl)-1H-pyrrolo[3,2-c]pyridin-6-yl)amino)-3,4-dihydroquinolin-2(1H)-one;
N-(3-((7-(2-(difluoromethoxy)benzyl)-7H-pyrrolo[2,3-d]pyrimidin-2-yl)amino)phenyl)acetamide;
N-(4-((7-((2-(N-methylmethylsulfonamido)pyridin-3-yl)methyl)-7H-pyrrolo[2,3-d]pyrimidin-2-yl)amino)phenyl)acetamide;
N-(7-(2-(difluoromethoxy)benzyl)-7H-pyrrolo[2,3-d]pyrimidin-2-yl)benzo[d]thiazol-6-amine;
N-(3-((1-(pyridin-2-yl)-1H-pyrrolo[3,2-c]pyridin-6-yl)amino)phenyl)acetamide;
N-methyl-3-((7-((2-(N-methylmethylsulfonamido)pyridin-3-yl)methyl)-7H-pyrrolo[2,3-d]pyrimidin-2-yl)amino)benzamide;
N-(3-((7-tosyl-7H-pyrrolo[2,3-d]pyrimidin-2-yl)amino)phenyl)acetamide;
N-(3-((7-((3-(trifluoromethoxy)phenyl)sulfonyl)-7H-pyrrolo[2,3-d]pyrimidin-2-yl)amino)phenyl)acetamide;
6-((7-((4-(trifluoromethoxy)phenyl)sulfonyl)-7H-pyrrolo[2,3-d]pyrimidin-2-yl)amino)-3,4-dihydroquinolin-2(1H)-one;
N-(3-((7-((2-(trifluoromethoxy)phenyl)sulfonyl)-7H-pyrrolo[2,3-d]pyrimidin-2-yl)amino)phenyl)acetamide;
6-((7-((2-(trifluoromethoxy)phenyl)sulfonyl)-7H-pyrrolo[2,3-d]pyrimidin-2-yl)amino)-3,4-dihydroquinolin-2(1H)-one;
N-(3-((7-((4-(trifluoromethoxy)phenyl)sulfonyl)-7H-pyrrolo[2,3-d]pyrimidin-2-yl)amino)phenyl)acetamide;
6-((7-((3-(trifluoromethoxy)phenyl)sulfonyl)-7H-pyrrolo[2,3-d]pyrimidin-2-yl)amino)-3,4-dihydroquinolin-2(1H)-one;
N-(3-((1-((4-(trifluoromethoxy)phenyl)sulfonyl)-1H-pyrrolo[3,2-c]pyridin-6-yl)amino)phenyl)acetamide;
N-(4-((1-((4-(trifluoromethoxy)phenyl)sulfonyl)-1H-pyrrolo[3,2-c]pyridin-6-yl)amino)phenyl)acetamide;
6-((1-((4-(trifluoromethoxy)phenyl)sulfonyl)-1H-pyrrolo[3,2-c]pyridin-6-yl)amino)-3,4-dihydroquinolin-2(1H)-one;
N-(1-((4-(trifluoromethoxy)phenyl)sulfonyl)-1H-pyrrolo[3,2-c]pyridin-6-yl)benzo[d]thiazol-6-amine;
N-methyl-N-(3-((2-((2-oxo-1,2,3,4-tetrahydroquinolin-6-yl)amino)-7H-pyrrolo[2,3-d]pyrimidin-7-yl)methyl)phenyl)methanesulfonamide;
N-(4-(methylsulfonyl)phenyl)-1-((3-(trifluoromethoxy)phenyl)sulfonyl)-1H-pyrrolo[3,2-c]pyridin-6-amine;
N-(3-((7-(2-(difluoromethoxy)benzoyl)-7H-pyrrolo[2,3-]pyrimidin-2-yl)amino)phenyl)acetamide;
N-(4-methoxyphenyl)-1-((3-(trifluoromethoxy)phenyl)sulfonyl)-1H-pyrrolo[3,2-c]pyridin-6-amine;
N-(4-((1-((3-(trifluoromethoxy)phenyl)sulfonyl)-1H-pyrrolo[3,2-c]pyridin-6-yl)amino)phenyl)acetamide;
N-(3-((1-((3-(trifluoromethoxy)phenyl)sulfonyl)-1H-pyrrolo[3,2-c]pyridin-6-yl)amino)phenyl)acetamide;
6-((1-((3-(trifluoromethoxy)phenyl)sulfonyl)-1H-pyrrolo[3,2-c]pyridin-6-yl)amino)-3,4-dihydroquinolin-2(1H)-one;
N-(4-(methylsulfonyl)phenyl)-1-((2-(trifluoromethoxy)phenyl)sulfonyl)-1H-pyrrolo[3,2-c]pyridin-6-amine;
N-(3-((1-((2-(trifluoromethoxy)phenyl)sulfonyl)-1H-pyrrolo[3,2-c]pyridin-6-yl)amino)phenyl)acetamide;
6-((1-((2-(trifluoromethoxy)phenyl)sulfonyl)-1H-pyrrolo[3,2-c]pyridin-6-yl)amino)-3,4-dihydroquinolin-2(1H)-one;
N-methyl-3-((2-((2-oxo-1,2,3,4-tetrahydroquinolin-6-yl)amino)-7H-pyrrolo[2,3-d]pyrimidin-7-yl)methyl)benzenesulfonamide;
N-(4-methoxy-2-methylphenyl)-7-(pyridin-3-ylmethyl)-7H-pyrrolo[2,3-d]pyrimidin-2-amine;
N-(4-((7-((2-(trifluoromethoxy)phenyl)sulfonyl)-7H-pyrrolo[2,3-d]pyrimidin-2-yl)amino)phenyl)acetamide;
N-(3-((2-(2-(difluoromethoxy)benzyl)-2H-pyrazolo[4,3-c]pyridin-6-yl)amino)phenyl)acetamide;
N-(3-((1-(2-(difluoromethoxy)benzyl)-1H-pyrazolo[4,3-c]pyridin-6-yl)amino)phenyl)acetamide;
6-((1-(2-(difluoromethoxy)benzyl)-1H-pyrazolo[4,3-c]pyridin-6-yl)amino)-3,4-dihydroquinolin-2(1H)-one;
N-(4-((1-((2-(N-methylmethylsulfonamido)pyridin-3-yl)methyl)-1H-pyrazolo[4,3-c]pyridin-6-yl)amino)phenyl)acetamide;
6-((2-(2-(difluoromethoxy)benzyl)-2H-pyrazolo[4,3-c]pyridin-6-yl)amino)-3,4-dihydroquinolin-2(1H)-one;
N-(3-((3-methyl-1-((2-(N-methylmethylsulfonamido)pyridin-3-yl)methyl)-1H-pyrazolo[4,3-c]pyridin-6-yl)amino)phenyl)acetamide;
N-methyl-N-(3-((3-methyl-6-((2-oxo-1,2,3,4-tetrahydroquinolin-6-yl)amino)-1H-pyrazolo[4,3-c]pyridin-1-yl)methyl)pyridin-2-yl)methanesulfonamide;
N-(6-((3-methyl-1-((2-(N-methylmethylsulfonamido)pyridin-3-yl)methyl)-1H-pyrazolo[4,3-c]pyridin-6-yl)amino)pyridin-3-yl)acetamide;
N-(2-((7-((2-(N-methylmethylsulfonamido)pyridin-3-yl)methyl)-7H-pyrrolo[2,3-d]pyrimidin-2-yl)amino)phenyl)acetamide;
N-(4-((3-methyl-1-((2-(N-methylmethylsulfonamido)pyridin-3-yl)methyl)-1H-pyrazolo[4,3-c]pyridin-6-yl)amino)phenyl)acetamide;
N-(6-((1-(2-(difluoromethoxy)benzyl)-1H-pyrazolo[4,3-c]pyridin-6-yl)amino)pyridin-3-yl)acetamide;
N-(3-((2-((1,3-dioxoisoindolin-5-yl)amino)-7H-pyrrolo[2,3-d]pyrimidin-7-yl)methyl)pyridin-2-yl)-N-methylmethanesulfonamide;
N-(4-((1-(2-(difluoromethoxy)benzyl)-1H-pyrazolo[4,3-c]pyridin-6-yl)amino)phenyl)acetamide;
N-methyl-N-(3-((2-((4-morpholinophenyl)amino)-7H-pyrrolo[2,3-d]pyrimidin-7-yl)methyl)pyridin-2-yl)methanesulfonamide;
N-methyl-N-(3-((2-((2-oxo-2,3-dihydro-1H-benzo[d]imidazol-5-yl)amino)-7H-pyrrolo[2,3-d]pyrimidin-7-yl)methyl)pyridin-2-yl)methanesulfonamide;
N-(3-((1-(2-(difluoromethoxy)benzyl)-3-methyl-1H-pyrazolo[4,3-c]pyridin-6-yl)amino)phenyl)acetamide;
2-hydroxy-2-methyl-N-(3-((7-((2-(N-methylmethylsulfonamido)pyridin-3-yl)methyl)-7H-pyrrolo[2,3-d]pyrimidin-2-yl)amino)phenyl)propanamide;
6-((1-(2-(difluoromethoxy)benzyl)-3-methyl-1H-pyrazolo[4,3-c]pyridin-6-yl)amino)-3,4-dihydroquinolin-2(1H)-one;
N-(4-((1-(2-(difluoromethoxy)benzyl)-3-methyl-1H-pyrazolo[4,3-c]pyridin-6-yl)amino)phenyl)acetamide;
N-(1-(2-(difluoromethoxy)benzyl)-1H-pyrazolo[4,3-c]pyridin-6-yl)benzo[d]thiazol-6-amine;
6-((7-(2-(trifluoromethoxy)benzyl)-7H-pyrrolo[2,3-d]pyrimidin-2-yl)amino)-3,4-dihydroquinolin-2(1H)-one;
N-(3-((1-((2-(N-methylmethylsulfonamido)pyridin-3-yl)methyl)-1H-pyrazolo[4,3-c]pyridin-6-yl)amino)phenyl)acetamide;
6-((1-((2-(trifluoromethoxy)phenyl)sulfonyl)-1H-pyrazolo[4,3-c]pyridin-6-yl)amino)-3,4-dihydroquinolin-2(1H)-one;
N-(3-((1-((2-(trifluoromethoxy)phenyl)sulfonyl)-1H-pyrazolo[4,3-c]pyridin-6-yl)amino)phenyl)acetamide;
N-(1-(2-(difluoromethoxy)benzyl)-3-methyl-1H-pyrazolo[4,3-c]pyridin-6-yl)benzo[d]thiazol-6-amine;
N-(3-((3-methyl-1-((2-(trifluoromethoxy)phenyl)sulfonyl)-1H-pyrazolo[4,3-c]pyridin-6-yl)amino)phenyl)acetamide;
N-(1-((2-(trifluoromethoxy)phenyl)sulfonyl)-1H-pyrazolo[4,3-c]pyridin-6-yl)benzo[d]thiazol-6-amine;
6-((3-methyl-1-((2-(trifluoromethoxy)phenyl)sulfonyl)-1H-pyrazolo[4,3-c]pyridin-6-yl)amino)-3,4-dihydroquinolin-2(1H)-one;
N-(3-((2-((3-(2-(dimethylamino)ethoxy)phenyl)amino)-7H-pyrrolo[2,3-d]pyrimidin-7-yl)methyl)pyridin-2-yl)-N-methylmethanesulfonamide;
N-(4-((1-((2-(trifluoromethoxy)phenyl)sulfonyl)-1H-pyrazolo[4,3-c]pyridin-6-yl)amino)phenyl)acetamide;
5-((7-((2-(trifluoromethoxy)phenyl)sulfonyl)-7H-pyrrolo[2,3-d]pyrimidin-2-yl)amino)-1H-benzo[d]imidazol-2(3H)-one;
1-(6-((7-((2-(trifluoromethoxy)phenyl)sulfonyl)-7H-pyrrolo[2,3-d]pyrimidin-2-yl)amino)pyridin-3-yl)piperazin-2-one;
N-(4-((7-(2-(difluoromethoxy)benzyl)-7H-pyrrolo[2,3-d]pyrimidin-2-yl)amino)phenyl)acetamide;
N-(4-fluoro-3-((7-((2-(N-methylmethylsulfonamido)pyridin-3-yl)methyl)-7H-pyrrolo[2,3-d]pyrimidin-2-yl)amino)phenyl)acetamide;
N-(4-((1-(2-(difluoromethoxy)benzyl)-1H-pyrrolo[3,2-c]pyridin-6-yl)amino)phenyl)acetamide;
N-(3-((1-(2-(difluoromethoxy)benzyl)-1H-pyrrolo[3,2-c]pyridin-6-yl)amino)phenyl)acetamide;
N-methyl-N-(3-((2-((3-morpholinophenyl)amino)-7H-pyrrolo[2,3-d]pyrimidin-7-yl)methyl)pyridin-2-yl)methanesulfonamide;
N-(3-((7-(2-(N-methylmethylsulfonamido)benzyl)-7H-pyrrolo[2,3-d]pyrimidin-2-yl)amino)phenyl)acetamide;
N-methyl-N-(3-((2-((4-(4-methylpiperazin-1-yl)phenyl)amino)-7H-pyrrolo[2,3-d]pyrimidin-7-yl)methyl)pyridin-2-yl)methanesulfonamide;
N-methyl-N-(3-((2-((4-(2-oxopiperazin-1-yl)phenyl)amino)-7H-pyrrolo[2,3-d]pyrimidin-7-yl)methyl)pyridin-2-yl)methanesulfonamide;
N-methyl-N-(2-((2-((2-oxo-1,2,3,4-tetrahydroquinolin-6-yl)amino)-7H-pyrrolo[2,3-d]pyrimidin-7-yl)methyl)phenyl)methanesulfonamide;
N-(3-((7-((2-methoxyphenyl)sulfonyl)-7H-pyrrolo[2,3-d]pyrimidin-2-yl)amino)phenyl)acetamide;
N-(3-((2-((4-(dimethylamino)phenyl)amino)-7H-pyrrolo[2,3-d]pyrimidin-7-yl)methyl)pyridin-2-yl)-N-methylmethanesulfonamide;
6-((7-((2-methoxyphenyl)sulfonyl)-7H-pyrrolo[2,3-d]pyrimidin-2-yl)amino)-3,4-dihydroquinolin-2(1H)-one;
N-methyl-N-(3-((2-((5-morpholinopyridin-2-yl)amino)-7H-pyrrolo[2,3-d]pyrimidin-7-yl)methyl)pyridin-2-yl)methanesulfonamide;
N-(3-((2-((1H-benzo[d]imidazol-5-yl)amino)-7H-pyrrolo[2,3-d]pyrimidin-7-yl)methyl)pyridin-2-yl)-N-methylmethanesulfonamide;
N-methyl-N-(3-((2-(phenylamino)-7H-pyrrolo[2,3-d]pyrimidin-7-yl)methyl)pyridin-2-yl)methanesulfonamide;
2-hydroxy-2-methyl-N-(3-((7-((2-(trifluoromethoxy)phenyl)sulfonyl)-7H-pyrrolo[2,3-d]pyrimidin-2-yl)amino)phenyl)propanamide;
N-methyl-N-(2-((2-((4-morpholinophenyl)amino)-7H-pyrrolo[2,3-d]pyrimidin-7-yl)methyl)phenyl)methanesulfonamide;
N-(4-morpholinophenyl)-7-((2-(trifluoromethoxy)phenyl)sulfonyl)-7H-pyrrolo[2,3-d]pyrimidin-2-amine;
N-(4-morpholinophenyl)-1-((2-(trifluoromethoxy)phenyl)sulfonyl)-1H-pyrrolo[3,2-c]pyridin-6-amine;
N-(3-((7-((2-cyanophenyl)sulfonyl)-7H-pyrrolo[2,3-d]pyrimidin-2-yl)amino)phenyl)acetamide;
2-((2-((2-oxo-1,2,3,4-tetrahydroquinolin-6-yl)amino)-7H-pyrrolo[2,3-d]pyrimidin-7-yl) sulfonyl)benzonitrile;
N-methyl-N-(3-((2-((4-(piperazin-1-yl)phenyl)amino)-7H-pyrrolo[2,3-d]pyrimidin-7-yl)methyl)pyridin-2-yl)methanesulfonamide;
7-(2-(difluoromethoxy)benzyl)-N-(3-(1,2,3,6-tetrahydropyridin-4-yl)-1H-indol-5-yl)-7H-pyrrolo[2,3-d]pyrimidin-2-amine hydrochloride;
5-((1-((2-(trifluoromethoxy)phenyl)sulfonyl)-1H-pyrrolo[3,2-c]pyridin-6-yl)amino)-1H-benzo[d]imidazol-2(3H)-one;
N-methyl-N-(3-((2-(pyridin-3-ylamino)-7H-pyrrolo[2,3-d]pyrimidin-7-yl)methyl)pyridin-2-yl)methanesulfonamide;
N-(3-((2-((4-fluorophenyl)amino)-7H-pyrrolo[2,3-d]pyrimidin-7-yl)methyl)pyridin-2-yl)-N-methylmethanesulfonamide;
N-(5-((7-((2-(N-methylmethylsulfonamido)pyridin-3-yl)methyl)-7H-pyrrolo[2,3-d]pyrimidin-2-yl)amino)pyridin-2-yl)acetamide;
N-(3-((2-((1H-indazol-6-yl)amino)-7H-pyrrolo[2,3-d]pyrimidin-7-yl)methyl)pyridin-2-yl)-N-methylmethanesulfonamide;
N-(3-((2-(benzo[d]thiazol-6-ylamino)-7H-pyrrolo[2,3-d]pyrimidin-7-yl)methyl)pyridin-2-yl)-N-methylmethanesulfonamide;
N-(3-((7-((2-(N-methylmethylsulfonamido)pyridin-3-yl)methyl)-7H-pyrrolo[2,3-d]pyrimidin-2-yl)oxy)phenyl)acetamide;
N-methyl-N-(3-((2-((4-(3-oxomorpholino)phenyl)amino)-7H-pyrrolo[2,3-d]pyrimidin-7-yl)methyl)pyridin-2-yl)methanesulfonamide;
N-(3-(2-(dimethylamino)ethoxy)phenyl)-1-((2-(trifluoromethoxy)phenyl)sulfonyl)-1H-pyrrolo[3,2-c]pyridin-6-amine;
N-(3-(2-(dimethylamino)ethoxy)phenyl)-7-((2-(trifluoromethoxy)phenyl)sulfonyl)-7H-pyrrolo[2,3-d]pyrimidin-2-amine;
N-(3-((2-((4-(2-(dimethylamino)ethoxy)phenyl)amino)-7H-pyrrolo[2,3-d]pyrimidin-7-yl)methyl)pyridin-2-yl)-N-methylmethanesulfonamide;
N-(7-((2-(trifluoromethoxy)phenyl)sulfonyl)-7H-pyrrolo[2,3-d]pyrimidin-2-yl)benzo[d]thiazol-6-amine;
6-((1-((2-methoxyphenyl)sulfonyl)-1H-pyrazolo[4,3-c]pyridin-6-yl)amino)-3,4-dihydroquinolin-2(1H)-one;
N-methyl-N-(3-((6-((2-oxo-1,2,3,4-tetrahydroquinolin-6-yl)amino)-1H-pyrazolo[4,3-c]pyridin-1-yl)methyl)pyridin-2-yl)methanesulfonamide;
N-(1-((2-methoxyphenyl)sulfonyl)-1H-pyrazolo[4,3-c]pyridin-6-yl)benzo[d]thiazol-6-amine;
N-(4-(3-(dimethylamino)pyrrolidin-1-yl)phenyl)-5,5-dimethyl-7-(quinolin-8-ylsulfonyl)-6,7-dihydro-5H-pyrrolo[2,3-d]pyrimidin-2-amine;
N-(3-((6-((3-(2-(dimethylamino)ethoxy)phenyl)amino)-1H-pyrazolo[4,3-c]pyridin-1-yl)methyl)pyridin-2-yl)-N-methylmethanesulfonamide;
N-(3-((6-(benzo[d]thiazol-6-ylamino)-1H-pyrazolo[4,3-c]pyridin-1-yl)methyl)pyridin-2-yl)-N-methylmethanesulfonamide;
N-methyl-N-(3-((2-((l-methyl-2-oxo-1,2,3,4-tetrahydroquinolin-6-yl)amino)-7H-pyrrolo[2,3-d]pyrimidin-7-yl)methyl)pyridin-2-yl)methanesulfonamide;
N,N-dimethyl-2-((2-((2-oxo-1,2,3,4-tetrahydroquinolin-6-yl)amino)-7H-pyrrolo[2,3-d]pyrimidin-7-yl)methyl)benzenesulfonamide;
N-methyl-N-(3-((2-(pyridin-4-ylamino)-7H-pyrrolo[2,3-d]pyrimidin-7-yl)methyl)pyridin-2-yl)methanesulfonamide;
N-(3-((2-((3-(dimethylamino)phenyl)amino)-7H-pyrrolo[2,3-d]pyrimidin-7-yl)methyl)pyridin-2-yl)-N-methylmethanesulfonamide;
N,N-dimethyl-2-((2-((3,4,5-trimethoxyphenyl)amino)-7H-pyrrolo[2,3-d]pyrimidin-7-yl)methyl)benzenesulfonamide;
N-methyl-N-(6-((2-((2-oxo-1,2,3,4-tetrahydroquinolin-6-yl)amino)-7H-pyrrolo[2,3-d]pyrimidin-7-yl)methyl)pyridin-2-yl)methanesulfonamide;
N-methyl-N-(3-((6-((2-oxo-2,3-dihydro-1H-benzo[d]imidazol-5-yl)amino)-1H-pyrazolo[4,3-c]pyridin-1-yl)methyl)pyridin-2-yl)methanesulfonamide;
7-((2-methoxyphenyl)sulfonyl)-N-(pyridin-3-yl)-7H-pyrrolo[2,3-d]pyrimidin-2-amine;
7-((2-methoxyphenyl)sulfonyl)-N-(pyridin-4-yl)-7H-pyrrolo[2,3-d]pyrimidin-2-amine;
N-methyl-N-(3-((6-((4-morpholinophenyl)amino)-1H-pyrazolo[4,3-c]pyridin-1-yl)methyl)pyridin-2-yl)methanesulfonamide;
7-((2-methoxyphenyl)sulfonyl)-N-(pyrimidin-5-yl)-7H-pyrrolo[2,3-d]pyrimidin-2-amine;
N-(3-((2-((3-fluorophenyl)amino)-7H-pyrrolo[2,3-d]pyrimidin-7-yl)methyl)pyridin-2-yl)-N-methylmethanesulfonamide;
N-methyl-N-(3-((2-(o-tolylamino)-7H-pyrrolo[2,3-d]pyrimidin-7-yl)methyl)pyridin-2-yl)methanesulfonamide;
6-((7-((2-fluorophenyl)sulfonyl)-7H-pyrrolo[2,3-d]pyrimidin-2-yl)amino)-3,4-dihydroquinolin-2(1H)-one;
N-(3-((2-((3-methoxyphenyl)amino)-7H-pyrrolo[2,3-d]pyrimidin-7-yl)methyl)pyridin-2-yl)-N-methylmethanesulfonamide;
6-((7-((2-(difluoromethoxy)phenyl)sulfonyl)-7H-pyrrolo[2,3-d]pyrimidin-2-yl)amino)-3,4-dihydroquinolin-2(1H)-one;
N-methyl-N-(3-((2-((3-(1,2,3,6-tetrahydropyridin-4-yl)-1H-indol-5-yl)amino)-7H-pyrrolo[2,3-d]pyrimidin-7-yl)methyl)pyridin-2-yl)methanesulfonamide hydrochloride;
N-(3-((2-((2-methoxyphenyl)amino)-7H-pyrrolo[2,3-d]pyrimidin-7-yl)methyl)pyridin-2-yl)-N-methylmethanesulfonamide;
N-(3-((2-((2-fluorophenyl)amino)-7H-pyrrolo[2,3-d]pyrimidin-7-yl)methyl)pyridin-2-yl)-N-methylmethanesulfonamide;
N-methyl-N-(3-((2-((4-(methylamino)phenyl)amino)-7H-pyrrolo[2,3-d]pyrimidin-7-yl)methyl)pyridin-2-yl)methanesulfonamide;
N-(3-((2-((4-cyanophenyl)amino)-7H-pyrrolo[2,3-d]pyrimidin-7-yl)methyl)pyridin-2-yl)-N-methylmethanesulfonamide;
6-((7-((2-(trifluoromethyl)phenyl)sulfonyl)-7H-pyrrolo[2,3-d]pyrimidin-2-yl)amino)-3,4-dihydroquinolin-2(1H)-one;
6-((1-((2-(difluoromethoxy)phenyl)sulfonyl)-1H-pyrazolo[4,3-c]pyridin-6-yl)amino)-3,4-dihydroquinolin-2(1H)-one;
N-methyl-N-(3-((2-(quinolin-5-ylamino)-7H-pyrrolo[2,3-d]pyrimidin-7-yl)methyl)pyridin-2-yl)methanesulfonamide;
6-((7-((2,5-dimethoxyphenyl)sulfonyl)-7H-pyrrolo[2,3-d]pyrimidin-2-yl)amino)-3,4-dihydroquinolin-2(1H)-one;
N-(3-((2-((4-(cyanomethyl)phenyl)amino)-7H-pyrrolo[2,3-d]pyrimidin-7-yl)methyl)pyridin-2-yl)-N-methylmethanesulfonamide;
N-methyl-N-(3-((2-(quinolin-6-ylamino)-7H-pyrrolo[2,3-d]pyrimidin-7-yl)methyl)pyridin-2-yl)methanesulfonamide;
6-((7-((2-aminophenyl)sulfonyl)-7H-pyrrolo[2,3-d]pyrimidin-2-yl)amino)-3,4-dihydroquinolin-2(1H)-one;
3-((7-((2-(N-methylmethylsulfonamido)pyridin-3-yl)methyl)-7H-pyrrolo[2,3-d]pyrimidin-2-yl)amino)benzenesulfonamide;
N-(3-((2-((3-(hydroxymethyl)phenyl)amino)-7H-pyrrolo[2,3-d]pyrimidin-7-yl)methyl)pyridin-2-yl)-N-methylmethanesulfonamide;
N-(3-((2-((2,6-difluorophenyl)amino)-7H-pyrrolo[2,3-d]pyrimidin-7-yl)methyl)pyridin-2-yl)-N-methylmethanesulfonamide;
N-methyl-N-(3-((2-((3-oxo-1,3-dihydroisobenzofuran-5-yl)amino)-7H-pyrrolo[2,3-d]pyrimidin-7-yl)methyl)pyridin-2-yl)methanesulfonamide;
6-((7-(quinolin-8-ylsulfonyl)-7H-pyrrolo[2,3-d]pyrimidin-2-yl)amino)-3,4-dihydroquinolin-2(1H)-one;
2-((6-((2-oxo-1,2,3,4-tetrahydroquinolin-6-yl)amino)-1H-pyrazolo[4,3-c]pyridin-1-yl)sulfonyl)benzonitrile;
N-methyl-N-(3-((6-((3-morpholinophenyl)amino)-1H-pyrazolo[4,3-c]pyridin-1-yl)methyl)pyridin-2-yl)methanesulfonamide;
6-((7-((3-methoxyphenyl)sulfonyl)-7H-pyrrolo[2,3-d]pyrimidin-2-yl)amino)-3,4-dihydroquinolin-2(1H)-one;
2-((2-((3-(2-(dimethylamino)ethoxy)phenyl)amino)-7H-pyrrolo[2,3-d]pyrimidin-7-yl)sulfonyl)benzonitrile;
N-methyl-2-((2-((3,4,5-trimethoxyphenyl)amino)-7H-pyrrolo[2,3-d]pyrimidin-7-yl)methyl)benzenesulfonamide;
N-(3-((2-((3-cyanophenyl)amino)-7H-pyrrolo[2,3-d]pyrimidin-7-yl)methyl)pyridin-2-yl)-N-methylmethanesulfonamide;
N-methyl-N-(3-((2-((3-(piperazin-1-yl)phenyl)amino)-7H-pyrrolo[2,3-d]pyrimidin-7-yl)methyl)pyridin-2-yl)methanesulfonamide;
2-((2-((3-fluorophenyl)amino)-7H-pyrrolo[2,3-d]pyrimidin-7-yl) sulfonyl)benzonitrile;
2-((2-((3-methoxyphenyl)amino)-7H-pyrrolo[2,3-d]pyrimidin-7-yl)sulfonyl)benzonitrile;
7-((2-(difluoromethoxy)phenyl)sulfonyl)-N-(3-fluorophenyl)-7H-pyrrolo[2,3-d]pyrimidin-2-amine;
5-((7-((2-(difluoromethoxy)phenyl)sulfonyl)-7H-pyrrolo[2,3-d]pyrimidin-2-yl)amino)-1H-benzo[d]imidazol-2(3H)-one;
7-((2-(difluoromethoxy)phenyl)sulfonyl)-N-(3-methoxyphenyl)-7H-pyrrolo[2,3-d]pyrimidin-2-amine;
N-methyl-2-((2-((2-oxo-1,2,3,4-tetrahydroquinolin-6-yl)amino)-7H-pyrrolo[2,3-d]pyrimidin-7-yl)methyl)benzenesulfonamide;
N-(4-((2-((4-(cyanomethyl)phenyl)amino)-7H-pyrrolo[2,3-d]pyrimidin-7-yl)sulfonyl)phenyl)acetamide;
7-((2-(difluoromethoxy)phenyl)sulfonyl)-N-(4-(piperazin-1-yl)phenyl)-7H-pyrrolo[2,3-d]pyrimidin-2-amine;
2-((2-((4-(piperazin-1-yl)phenyl)amino)-7H-pyrrolo[2,3-d]pyrimidin-7-yl) sulfonyl)benzonitrile;
N-methyl-N-(3-((2-((4-(piperazin-1-yl)phenyl)amino)-7H-pyrrolo[2,3-d]pyrimidin-7-yl)methyl)pyridin-2-yl)methanesulfonamide hydrochloride;
6-((1-(quinolin-8-ylsulfonyl)-1H-pyrazolo[4,3-c]pyridin-6-yl)amino)-3,4-dihydroquinolin-2(1H)-one;
N-(3-((2-((3-(1H-tetrazol-5-yl)phenyl)amino)-7H-pyrrolo[2,3-d]pyrimidin-7-yl)methyl)pyridin-2-yl)-N-methylmethanesulfonamide;
N-(3-((2-((1H-benzo[d][1,2,3]triazol-6-yl)amino)-7H-pyrrolo[2,3-d]pyrimidin-7-yl)methyl)pyridin-2-yl)-N-methylmethanesulfonamide;
N-methyl-N-(3-((6-((2-oxo-1,2,3,4-tetrahydroquinolin-6-yl)amino)-2H-pyrazolo[3,4-d]pyrimidin-2-yl)methyl)pyridin-2-yl)methanesulfonamide;
2-((6-((4-(piperazin-1-yl)phenyl)amino)-1H-pyrazolo[4,3-c]pyridin-1-yl)sulfonyl)benzonitrile;
N-(4-((2-((2-oxo-1,2,3,4-tetrahydroquinolin-6-yl)amino)-7H-pyrrolo[2,3-d]pyrimidin-7-yl)sulfonyl)phenyl)acetamide;
N-(4-(piperazin-1-yl)phenyl)-7-(quinolin-8-ylsulfonyl)-7H-pyrrolo[2,3-d]pyrimidin-2-amine;
N-(3-fluoro-4-(piperazin-1-yl)phenyl)-7-(quinolin-8-ylsulfonyl)-7H-pyrrolo[2,3-d]pyrimidin-2-amine;
N-(3-((2-((3-fluoro-4-(piperazin-1-yl)phenyl)amino)-7H-pyrrolo[2,3-d]pyrimidin-7-yl)methyl)pyridin-2-yl)-N-methylmethanesulfonamide;
N-(3-methyl-4-(piperazin-1-yl)phenyl)-7-(quinolin-8-ylsulfonyl)-7H-pyrrolo[2,3-d]pyrimidin-2-amine;
N-methyl-N-(3-((2-((3-methyl-4-(piperazin-1-yl)phenyl)amino)-7H-pyrrolo[2,3-d]pyrimidin-7-yl)methyl)pyridin-2-yl)methanesulfonamide;
N-(3-methoxy-4-(piperazin-1-yl)phenyl)-7-(quinolin-8-ylsulfonyl)-7H-pyrrolo[2,3-d]pyrimidin-2-amine;
6-((7-((2-chlorophenyl)sulfonyl)-7H-pyrrolo[2,3-d]pyrimidin-2-yl)amino)-3,4-dihydroquinolin-2(1H)-one;
N,N-dimethyl-2-((2-((4-(piperazin-1-yl)phenyl)amino)-7H-pyrrolo[2,3-d]pyrimidin-7-yl)methyl)benzenesulfonamide;
N-(3-((2-((4-((2-hydroxyethyl)amino)phenyl)amino)-7H-pyrrolo[2,3-d]pyrimidin-7-yl)methyl)pyridin-2-yl)-N-methylmethanesulfonamide;
N-methyl-N-{3-[6-(2-oxo-1,2,3,4-tetrahydro-quinolin-6-ylamino)-pyrazolo[3,4-d]pyrimidin-1-ylmethyl]-pyridin-2-yl}-methanesulfonamide;
N-methyl-N-{3-[2-(3-oxo-3,4-dihydro-2H-benzo[1,4]oxazin-6-ylamino)-pyrrolo[2,3-d]pyrimidin-7-ylmethyl]-pyridin-2-yl}-methanesulfonamide;
N,N-dimethyl-2-[6-(2-oxo-1,2,3,4-tetrahydro-quinolin-6-ylamino)-pyrazolo[4,3-c]pyridin-1-ylmethyl]-benzene sulfonamide;
(4-dimethylaminomethyl-phenyl)-[7-(quinoline-8-sulfonyl)-7H-pyrrolo[2,3-d]pyrimidin-2-yl]-amine;
N-methyl-N-(3-{2-[4-(1,2,3,6-tetrahydro-pyridin-4-yl)-phenylamino]-pyrrolo[2,3-d]pyrimidin-7-ylmethyl}-pyridin-2-yl)-methanesulfonamide;
N-{3-[2-(4-dimethylaminomethyl-phenylamino)-pyrrolo[2,3-d]pyrimidin-7-ylmethyl]-pyridin-2-yl}-N-methyl-methanesulfonamide;
N,N-dimethyl-2-[6-(4-piperazin-1-yl-phenylamino)-pyrazolo[4,3-c]pyridin-1-ylmethyl]-benzene sulfonamide;
N-methyl-N-(3-((2-((2-oxo-1,2,3,4-tetrahydroquinolin-7-yl)amino)-7H-pyrrolo[2,3-d]pyrimidin-7-yl)methyl)pyridin-2-yl)methanesulfonamide;
N-(4-(piperazin-1-yl)phenyl)-1-(quinolin-8-ylsulfonyl)-1H-pyrazolo[4,3-c]pyridin-6-amine;
N-{3-[2-(4,4-dimethyl-2-oxo-1,2,3,4-tetrahydro-quinolin-6-ylamino)-pyrrolo[2,3-d]pyrimidin-7-ylmethyl]-pyridin-2-yl}-N-methyl-methanesulfonamide;
2-[2-(3-methyl-4-piperazin-1-yl-phenylamino)-pyrrolo[2,3-d]pyrimidine-7-sulfonyl]-benzonitrile;
[4-((R)-3-methyl-piperazin-1-yl)-phenyl]-[7-(quinoline-8-sulfonyl)-7H-pyrrolo[2,3-d]pyrimidin-2-yl]-amine;
N-methyl-N-(2-((2-((4-(piperazin-1-yl)phenyl)amino)-7H-pyrrolo[2,3-d]pyrimidin-7-yl)methyl)phenyl)methanesulfonamide;
N-{3-[2-(3,4-dihydro-2H-benzo[1,4]oxazin-6-ylamino)-pyrrolo[2,3-d]pyrimidin-7-ylmethyl]-pyridin-2-yl}-N-methyl-methanesulfonamide;
6-((7-((2-(1, 1-dioxidoisothiazolidin-2-yl)pyridin-3-yl)methyl)-7H-pyrrolo[2,3-d]pyrimidin-2-yl)amino)-3,4-dihydroquinolin-2(1H)-one;
N-methyl-N-{3-[6-(4-piperazin-1-yl-phenylamino)-pyrazolo[4,3-c]pyridin-1-ylmethyl]-pyridin-2-yl}-methanesulfonamide;
N-methyl-N-(3-((2-((3-oxo-3,4-dihydro-2H-benzo[b][1,4]oxazin-7-yl)amino)-7H-pyrrolo[2,3-d]pyrimidin-7-yl)methyl)pyridin-2-yl)methanesulfonamide;
[7-(naphthalene-1-sulfonyl)-7H-pyrrolo[2,3-d]pyrimidin-2-yl]-(4-piperazin-1-yl-phenyl)-amine;
2-[6-(2-oxo-1,2,3,4-tetrahydro-quinolin-6-ylamino)-pyrazolo[3,4-d]pyrimidine-1-sulfonyl]-benzonitrile;
N-(3-((2-((3,4-dihydro-2H-benzo[b][1,4]oxazin-7-yl)amino)-7H-pyrrolo[2,3-d]pyrimidin-7-yl)methyl)pyridin-2-yl)-N-methylmethanesulfonamide;
N-methyl-N-(2-((6-((4-(piperazin-1-yl)phenyl)amino)-1H-pyrazolo[4,3-c]pyridin-1-yl)methyl)phenyl)methanesulfonamide;
6-[7-(naphthalene-1-sulfonyl)-7H-pyrrolo[2,3-d]pyrimidin-2-ylamino]-3,4-dihydro-1H-quinolin-2-one;
(7-benzenesulfonyl-7H-pyrrolo[2,3-d]pyrimidin-2-yl)-(4-piperazin-1-yl-phenyl)-amine;
N-methyl-N-{2-[6-(2-oxo-1,2,3,4-tetrahydro-quinolin-6-ylamino)-pyrazolo[4,3-c]pyridin-1-ylmethyl]-phenyl}-methanesulfonamide;
6-((7-(benzo[d]thiazol-6-ylsulfonyl)-7H-pyrrolo[2,3-d]pyrimidin-2-yl)amino)-3,4-dihydroquinolin-2(1H)-one;
N-methyl-N-{3-[2-(pyrimidin-2-ylamino)-pyrrolo[2,3-d]pyrimidin-7-ylmethyl]-pyridin-2-yl}-methanesulfonamide;
6-[1-(quinoline-8-sulfonyl)-1H-pyrazolo[3,4-d]pyrimidin-6-ylamino]-3,4-dihydro-1H-quinolin-2-one;
(3-methyl-4-piperazin-1-yl-phenyl)-[7-(thiophene-2-sulfonyl)-7H-pyrrolo[2,3-d]pyrimidin-2-yl]-amine;
[4-((S)-3-methyl-piperazin-1-yl)-phenyl]-[7-(quinoline-8-sulfonyl)-7H-pyrrolo[2,3-d]pyrimidin-2-yl]-amine;
N-(4-(piperazin-1-yl)phenyl)-1-(quinolin-8-ylsulfonyl)-1H-pyrazolo[3,4-d]pyrimidin-6-amine;
[7-(2-chloro-benzenesulfonyl)-7H-pyrrolo[2,3-d]pyrimidin-2-yl]-(4-piperazin-1-yl-phenyl)-amine;
(4-piperazin-1-yl-phenyl)-[7-(toluene-2-sulfonyl)-7H-pyrrolo[2,3-d]pyrimidin-2-yl]-amine;
[7-(benzothiazole-6-sulfonyl)-7H-pyrrolo[2,3-d]pyrimidin-2-yl]-(4-piperazin-1-yl-phenyl)-amine;
[7-(isoquinoline-5-sulfonyl)-7H-pyrrolo[2,3-d]pyrimidin-2-yl]-(4-piperazin-1-yl-phenyl)-amine;
[7-(1-methyl-1H-imidazole-4-sulfonyl)-7H-pyrrolo[2,3-d]pyrimidin-2-yl]-(4-piperazin-1-yl-phenyl)-amine;
[7-(1,2-dimethyl-1H-imidazole-4-sulfonyl)-7H-pyrrolo[2,3-d]pyrimidin-2-yl]-(4-piperazin-1-yl-phenyl)-amine;
(7-cyclopentanesulfonyl-7H-pyrrolo[2,3-d]pyrimidin-2-yl)-(4-piperazin-1-yl-phenyl)-amine;
(4-piperazin-1-yl-phenyl)-[7-(thiophene-2-sulfonyl)-7H-pyrrolo[2,3-d]pyrimidin-2-yl]-amine;
2-[2-(4-piperazin-1-ylmethyl-phenylamino)-pyrrolo[2,3-d]pyrimidine-7-sulfonyl]-benzonitrile;
N-methyl-N-{3-[6-(4-piperazin-1-yl-phenylamino)-pyrazolo[3,4-d]pyrimidin-1-ylmethyl]-pyridin-2-yl}-methanesulfonamide;
2-{2-[4-((S)-3-methyl-piperazin-1-yl)-phenylamino]-pyrrolo[2,3-d]pyrimidine-7-sulfonyl}-benzonitrile;
2-{2-[4-((R)-3-methyl-piperazin-1-yl)-phenylamino]-pyrrolo[2,3-d]pyrimidine-7-sulfonyl}-benzonitrile;
(4-piperazin-1-ylmethyl-phenyl)-[7-(quinoline-8-sulfonyl)-7H-pyrrolo[2,3-d]pyrimidin-2-yl]-amine;
2-[2-(3-fluoro-4-piperazin-1-yl-phenylamino)-pyrrolo[2,3-d]pyrimidine-7-sulfonyl]-benzonitrile;
[4-(2,5-diaza-bicyclo[2.2.1]hept-2-yl)-phenyl]-[7-(quinoline-8-sulfonyl)-7H-pyrrolo[2,3-d]pyrimidin-2-yl]-amine;
(1-{4-[7-(quinoline-8-sulfonyl)-7H-pyrrolo[2,3-d]pyrimidin-2-ylamino]-benzyl}-piperidin-3-yl)-methanol;
[4-(4,4-difluoro-piperidin-1-ylmethyl)-phenyl]-[7-(quinoline-8-sulfonyl)-7H-pyrrolo[2,3-d]pyrimidin-2-yl]-amine;
2-{2-[4-(4,4-difluoro-piperidin-1-ylmethyl)-phenylamino]-pyrrolo[2,3-d]pyrimidine-7-sulfonyl}-benzonitrile;
2-((2-((4-((3-hydroxypiperidin-1-yl)methyl)phenyl)amino)-7H-pyrrolo[2,3-]pyrimidin-7-yl)sulfonyl)benzonitrile;
2-(4-(4-((7-((2-cyanophenyl)sulfonyl)-7H-pyrrolo[2,3-d]pyrimidin-2-yl)amino)phenyl)piperidin-1-yl)acetamide;
(4-piperazin-1-yl-phenyl)-[7-(pyridine-3-sulfonyl)-7H-pyrrolo[2,3-d]pyrimidin-2-yl]-amine;
7-(benzo[c][1,2,5]thiadiazol-4-ylsulfonyl)-N-(4-(piperazin-1-yl)phenyl)-7H-pyrrolo[2,3-d]pyrimidin-2-amine;
[1-(2-chloro-benzenesulfonyl)-1H-pyrazolo[3,4-d]pyrimidin-6-yl]-(4-piperazin-1-yl-phenyl)-amine;
(1-benzenesulfonyl-1H-pyrazolo[3,4-d]pyrimidin-6-yl)-(4-piperazin-1-yl-phenyl)-amine;
2-(4-(4-((7-(quinolin-8-ylsulfonyl)-7H-pyrrolo[2,3-d]pyrimidin-2-yl)amino)phenyl)piperidin-1-yl)acetamide;
(4-pyrrolidin-1-ylmethyl-phenyl)-[7-(quinoline-8-sulfonyl)-7H-pyrrolo[2,3-d]pyrimidin-2-yl]-amine;
2-[2-(4-pyrrolidin-1-ylmethyl-phenylamino)-pyrrolo[2,3-d]pyrimidine-7-sulfonyl]-benzonitrile;
(4-piperidin-4-yl-phenyl)-[7-(quinoline-8-sulfonyl)-7H-pyrrolo[2,3-d]pyrimidin-2-yl]-amine;
[7-(2-chloro-6-methyl-benzenesulfonyl)-7H-pyrrolo[2,3-d]pyrimidin-2-yl]-(4-piperazin-1-yl-phenyl)-amine;
[3-fluoro-4-((S)-3-methyl-piperazin-1-yl)-phenyl]-[7-(quinoline-8-sulfonyl)-7H-pyrrolo[2,3-d]pyrimidin-2-yl]-amine;
[3-fluoro-4-((R)-3-methyl-piperazin-1-yl)-phenyl]-[7-(quinoline-8-sulfonyl)-7H-pyrrolo[2,3-d]pyrimidin-2-yl]-amine;
(7-cyclopropanesulfonyl-7H-pyrrolo[2,3-d]pyrimidin-2-yl)-(4-piperazin-1-yl-phenyl)-amine;
[7-(2,3-dichloro-benzenesulfonyl)-7H-pyrrolo[2,3-d]pyrimidin-2-yl]-(4-piperazin-1-yl-phenyl)-amine;
[7-(3-chloro-benzenesulfonyl)-7H-pyrrolo[2,3-d]pyrimidin-2-yl]-(4-piperazin-1-yl-phenyl)-amine;
2-{2-[4-(4-acetyl-piperazin-1-yl)-phenylamino]-pyrrolo[2,3-d]pyrimidine-7-sulfonyl}-benzonitrile;
2-((6-((3-fluoro-4-(piperazin-1-yl)phenyl)amino)-1H-pyrazolo[4,3-c]pyridin-1-yl)sulfonyl)benzonitrile;
1-(4-{4-[7-(quinoline-8-sulfonyl)-7H-pyrrolo[2,3-d]pyrimidin-2-ylamino]-phenyl}-piperazin-1-yl)-ethanone;
((R)-1-{4-[7-(quinoline-8-sulfonyl)-7H-pyrrolo[2,3-d]pyrimidin-2-ylamino]-benzyl}-pyrrolidin-2-yl)-methanol;
[1-(2-difluoromethoxy-benzenesulfonyl)-1H-pyrazolo[3,4-d]pyrimidin-6-yl]-(4-piperazin-1-yl-phenyl)-amine;
2-{6-[4-((R)-3-methyl-piperazin-1-yl)-phenylamino]-pyrazolo[3,4-d]pyrimidine-1-sulfonyl}-benzonitrile;
piperazin-1-yl-{4-[7-(quinoline-8-sulfonyl)-7H-pyrrolo[2,3-d]pyrimidin-2-ylamino]-phenyl}-methanone;
(3-methoxy-4-piperazin-1-yl-phenyl)-[1-(quinoline-8-sulfonyl)-1H-pyrazolo[4,3-c]pyridin-6-yl]-amine;
2-[6-(3-methoxy-4-piperazin-1-yl-phenylamino)-pyrazolo[4,3-c]pyridine-1-sulfonyl]-benzonitrile;
2-[6-(3-methyl-4-piperazin-1-yl-phenylamino)-pyrazolo[4,3-c]pyridine-1-sulfonyl]-benzonitrile;
2-{6-[4-(piperazine-1-carbonyl)-phenylamino]-pyrazolo[4,3-c]pyridine-1-sulfonyl}-benzonitrile;
piperazin-1-yl-{4-[1-(quinoline-8-sulfonyl)-1H-pyrazolo[4,3-c]pyridin-6-ylamino]-phenyl}-methanone;
2-{6-[4-((S)-3-methyl-piperazin-1-yl)-phenylamino]-pyrazolo[3,4-d]pyrimidine-1-sulfonyl}-benzonitrile;
(R)-2-((6-((4-(3-methylpiperazin-1-yl)phenyl)amino)-1H-pyrazolo[4,3-c]pyridin-1-yl)sulfonyl)benzonitrile;
[4-((S)-3-methyl-piperazin-1-yl)-phenyl]-[1-(quinoline-8-sulfonyl)-1H-pyrazolo[4,3-c]pyridin-6-yl]-amine;
N-(4-(piperazin-1-yl)phenyl)-1-(thiophen-2-ylsulfonyl)-1H-pyrazolo[4,3-c]pyridin-6-amine;
[4-((R)-3-methyl-piperazin-1-yl)-phenyl]-[1-(quinoline-8-sulfonyl)-1H-pyrazolo[4,3-c]pyridin-6-yl]-amine;
[1-(2-chloro-benzenesulfonyl)-1H-pyrazolo[3,4-d]pyrimidin-6-yl]-[4-((R)-3-methyl-piperazin-1-yl)-phenyl]-amine
[4-((R)-3-methyl-piperazin-1-yl)-phenyl]-[1-(quinoline-8-sulfonyl)-1H-pyrazolo[3,4-d]pyrimidin-6-yl]-amine;
(3-fluoro-4-piperazin-1-yl-phenyl)-[1-(quinoline-8-sulfonyl)-1H-pyrazolo[3,4-d]pyrimidin-6-yl]-amine;
N-(3-fluoro-4-(piperazin-1-yl)phenyl)-1-(quinolin-8-ylsulfonyl)-1H-pyrazolo[4,3-c]pyridin-6-amine;
2-{6-[4-(2,5-diaza-bicyclo[2.2.1]hept-2-yl)-phenylamino]-pyrazolo[4,3-c]pyridine-1-sulfonyl}-benzonitrile;
[3-fluoro-4-((R)-3-methyl-piperazin-1-yl)-phenyl]-[1-(quinoline-8-sulfonyl)-1H-pyrazolo[4,3-c]pyridin-6-yl]-amine;
2-[6-(4-piperazin-1-ylmethyl-phenylamino)-pyrazolo[4,3-c]pyridine-1-sulfonyl]-benzonitrile;
[5-fluoro-7-(quinoline-8-sulfonyl)-7H-pyrrolo[2,3-d]pyrimidin-2-yl]-(4-piperazin-1-yl-phenyl)-amine;
[7-(2,5-dichloro-benzenesulfonyl)-7H-pyrrolo[2,3-d]pyrimidin-2-yl]-(4-piperazin-1-yl-phenyl)-amine;
[1-(2-chloro-benzenesulfonyl)-1H-pyrazolo[3,4-d]pyrimidin-6-yl]-[4-((S)-3-methyl-piperazin-1-yl)-phenyl]-amine;
N-(4-(piperidin-4-yl)phenyl)-1-(quinolin-8-ylsulfonyl)-1H-pyrazolo[4,3-c]pyridin-6-amine;
[4-((S)-3-methyl-piperazin-1-yl)-phenyl]-[1-(quinoline-8-sulfonyl)-1H-pyrazolo[3,4-d]pyrimidin-6-yl]-amine;
[1-(2-chloro-benzenesulfonyl)-1H-pyrazolo[3,4-d]pyrimidin-6-yl]-(3-fluoro-4-piperazin-1-yl-phenyl)-amine;
(3-methyl-4-piperazin-1-yl-phenyl)-[1-(quinoline-8-sulfonyl)-1H-pyrazolo[4,3-c]pyridin-6-yl]-amine;
2-{6-[3-fluoro-4-((R)-3-methyl-piperazin-1-yl)-phenylamino]-pyrazolo[4,3-c]pyridine-1-sulfonyl}-benzonitrile;
1-(4-{4-[1-(quinoline-8-sulfonyl)-1H-pyrazolo[3,4-d]pyrimidin-6-ylamino]-phenyl}-piperazin-1-yl)-ethanone;
[1-(2-fluoro-benzenesulfonyl)-1H-pyrazolo[3,4-d]pyrimidin-6-yl]-[4-((S)-3-methyl-piperazin-1-yl)-phenyl]-amine;
3-((6-((4-(piperazin-1-yl)phenyl)amino)-1H-pyrazolo[4,3-c]pyridin-1-yl)sulfonyl)benzonitrile;
[7-(2-chloro-benzenesulfonyl)-7H-pyrrolo[2,3-d]pyrimidin-2-yl]-[4-((S)-3-methyl-piperazin-1-yl)-phenyl]-amine;
[1-(2-fluoro-benzenesulfonyl)-1H-pyrazolo[3,4-d]pyrimidin-6-yl]-[4-((R)-3-methyl-piperazin-1-yl)-phenyl]-amine;
5-[7-(quinoline-8-sulfonyl)-7H-pyrrolo[2,3-]pyrimidin-2-ylamino]-1,3-dihydro-indol-2-one;
N-methyl-N-{3-[2-(2-oxo-2,3-dihydro-1H-indol-5-ylamino)-pyrrolo[2,3-d]pyrimidin-7-ylmethyl]-pyridin-2-yl}-methanesulfonamide;
3-[2-(4-piperazin-1-yl-phenylamino)-pyrrolo[2,3-d]pyrimidine-7-sulfonyl]-benzonitrile;
3-{2-[4-((S)-3-methyl-piperazin-1-yl)-phenylamino]-pyrrolo[2,3-d]pyrimidine-7-sulfonyl}-benzonitrile;
N-{4-[7-(quinoline-8-sulfonyl)-7H-pyrrolo[2,3-d]pyrimidin-2-ylamino]-phenyl}-acetamide;
1-((3-chlorophenyl)sulfonyl)-N-(4-(piperazin-1-yl)phenyl)-1H-pyrazolo[4,3-c]pyridin-6-amine;
N-(4-((1-((2-cyanophenyl)sulfonyl)-1H-pyrazolo[4,3-c]pyridin-6-yl)amino)phenyl)acetamide;
1-{4-[7-(quinoline-8-sulfonyl)-7H-pyrrolo[2,3-d]pyrimidin-2-ylamino]-phenyl}-piperidin-4-ol;
2-(4-{4-[7-(quinoline-8-sulfonyl)-7H-pyrrolo[2,3-d]pyrimidin-2-ylamino]-phenyl}-piperazin-1-yl)-ethanol;
cyclopropyl-(4-{4-[7-(quinoline-8-sulfonyl)-7H-pyrrolo[2,3-d]pyrimidin-2-ylamino]-phenyl}-piperazin-1-yl)-methanone;
N-methyl-N-{3-[6-(4-piperazin-1-yl-phenylamino)-pyrrolo[3,2-c]pyridin-1-ylmethyl]-pyridin-2-yl}-methanesulfonamide;
2-((6-((4-(piperazin-1-yl)phenyl)amino)-1H-pyrrolo[3,2-c]pyridin-1-yl)sulfonyl)benzonitrile;
N-{4-[1-(quinoline-8-sulfonyl)-1H-pyrazolo[4,3-c]pyridin-6-ylamino]-phenyl}-acetamide;
2-[2-(2-oxo-2,3-dihydro-H-indol-5-ylamino)-pyrrolo[2,3-d]pyrimidine-7-sulfonyl]-benzonitrile;
2-[2-(1-methyl-1H-pyrazol-4-ylamino)-pyrrolo[2,3-d]pyrimidine-7-sulfonyl]-benzonitrile;
(1-methyl-1H-pyrazol-4-yl)-[7-(quinoline-8-sulfonyl)-7H-pyrrolo[2,3-d]pyrimidin-2-yl]-amine;
2-[5-fluoro-2-(4-piperazin-1-yl-phenylamino)-pyrrolo[2,3-d]pyrimidine-7-sulfonyl]-benzonitrile;
N-methyl-N-(3-{2-[4-((S)-2-methyl-piperazin-1-yl)-phenylamino]-pyrrolo[2,3-d]pyrimidin-7-ylmethyl}-pyridin-2-yl)-methanesulfonamide;
N-(4-(piperazin-1-yl)phenyl)-1-(quinolin-8-ylsulfonyl)-1H-pyrrolo[3,2-c]pyridin-6-amine;
N-(2-((6-((3-fluoro-4-(piperazin-1-yl)phenyl)amino)-1H-pyrrolo[3,2-c]pyridin-1-yl)methyl)phenyl)-N-methylmethanesulfonamide;
(3-fluoro-4-piperazin-1-yl-phenyl)-[1-(quinoline-8-sulfonyl)-1H-pyrrolo[3,2-c]pyridin-6-yl]-amine;
N-{4-[7-(quinoline-8-sulfonyl)-7H-pyrrolo[2,3-d]pyrimidin-2-ylamino]-phenyl}-isobutyramide;
N-methyl-N-(2-((6-((4-(piperazin-1-yl)phenyl)amino)-1H-pyrrolo[3,2-c]pyridin-1-yl)methyl)phenyl)methanesulfonamide;
[4-((S)-2-methyl-piperazin-1-yl)-phenyl]-[7-(quinoline-8-sulfonyl)-7H-pyrrolo[2,3-d]pyrimidin-2-yl]-amine;
(2-methoxy-4-piperazin-1-yl-phenyl)-[7-(quinoline-8-sulfonyl)-7H-pyrrolo[2,3-d]pyrimidin-2-yl]-amine;
(3-methoxy-4-piperazin-1-yl-phenyl)-[1-(quinoline-8-sulfonyl)-1H-pyrrolo[3,2-c]pyridin-6-yl]-amine;
[4-(2,5-diaza-bicyclo[2.2.1]hept-2-yl)-phenyl]-[1-(quinoline-8-sulfonyl)-1H-pyrrolo[3,2-c]pyridin-6-yl]-amine;
N-(2-methoxy-4-(piperazin-1-yl)phenyl)-1-(quinolin-8-ylsulfonyl)-1H-pyrrolo[3,2-c]pyridin-6-amine;
cyclopropanecarboxylic acid {4-[7-(quinoline-8-sulfonyl)-7H-pyrrolo[2,3-d]pyrimidin-2-ylamino]-phenyl}-amide;
(3-methyl-4-piperazin-1-yl-phenyl)-[1-(quinoline-8-sulfonyl)-1H-pyrazolo[3,4-d]pyrimidin-6-yl]-amine;
[7-(2-methoxy-benzenesulfonyl)-7H-pyrrolo[2,3-d]pyrimidin-2-yl]-(4-piperazin-1-yl-phenyl)-amine;
(3-methyl-4-piperazin-1-yl-phenyl)-[1-(quinoline-8-sulfonyl)-1H-pyrrolo[3,2-c]pyridin-6-yl]-amine;
[1-(2-methoxy-benzenesulfonyl)-1H-pyrazolo[3,4-d]pyrimidin-6-yl]-(4-piperazin-1-yl-phenyl)-amine;
2-[2-(2-methoxy-4-piperazin-1-yl-phenylamino)-pyrrolo[2,3-d]pyrimidine-7-sulfonyl]-benzonitrile;
N-{3-[2-(2-methoxy-4-piperazin-1-yl-phenylamino)-pyrrolo[2,3-d]pyrimidin-7-ylmethyl]-pyridin-2-yl}-N-methyl-methanesulfonamide;
N-(2-methoxy-4-(piperazin-1-yl)phenyl)-1-(quinolin-8-ylsulfonyl)-1H-pyrazolo[4,3-c]pyridin-6-amine;
(1-methyl-1H-pyrazol-4-yl)-[1-(quinoline-8-sulfonyl)-H-pyrrolo[3,2-c]pyridin-6-yl]-amine;
2-(4-{4-[1-(quinoline-8-sulfonyl)-1H-pyrrolo[3,2-c]pyridin-6-ylamino]-phenyl}-piperazin-1-yl)-ethanol;
2-{6-[4-(4-acetyl-piperazin-1-yl)-phenylamino]-pyrazolo[4,3-c]pyridine-1-sulfonyl}-benzonitrile;
1-((2-methoxyphenyl)sulfonyl)-N-(4-(piperazin-1-yl)phenyl)-1H-pyrazolo[4,3-c]pyridin-6-amine;
[1-(2-methoxy-benzenesulfonyl)-1H-pyrrolo[3,2-c]pyridin-6-yl]-(4-piperazin-1-yl-phenyl)-amine;
[7-(2-methoxy-benzenesulfonyl)-6,7-dihydro-5H-pyrrolo[2,3-d]pyrimidin-2-yl]-(4-piperazin-1-yl-phenyl)-amine;
1-(4-((1-(quinolin-8-ylsulfonyl)-1H-pyrazolo[4,3-c]pyridin-6-yl)amino)phenyl)piperidin-4-ol;
1-{4-[1-(quinoline-8-sulfonyl)-1H-pyrrolo[3,2-c]pyridin-6-ylamino]-phenyl}-piperidin-4-ol;
2-(4-{3-methoxy-4-[7-(quinoline-8-sulfonyl)-7H-pyrrolo[2,3-d]pyrimidin-2-ylamino]-phenyl}-piperazin-1-yl)-ethanol;
2-[6-(2-methoxy-4-piperazin-1-yl-phenylamino)-pyrrolo[3,2-c]pyridine-1-sulfonyl]-benzonitrile;
(1-methyl-1H-pyrazol-4-yl)-[1-(quinoline-8-sulfonyl)-1H-pyrazolo[4,3-c]pyridin-6-yl]-amine;
cyclopropyl-(4-{4-[7-(2-methoxy-benzenesulfonyl)-7H-pyrrolo[2,3-d]pyrimidin-2-ylamino]-phenyl}-piperazin-1-yl)-methanone;
2-(4-(4-((1-(quinolin-8-ylsulfonyl)-1H-pyrazolo[4,3-c]pyridin-6-yl)amino)phenyl)piperazin-1-yl)ethanol;
cyclopropyl(4-(4-((1-(quinolin-8-ylsulfonyl)-1H-pyrazolo[4,3-c]pyridin-6-yl)amino)phenyl)piperazin-1-yl)methanone;
N-(4-(4-methylpiperazin-1-yl)phenyl)-1-(quinolin-8-ylsulfonyl)-1H-pyrrolo[3,2-c]pyridin-6-amine;
5-[1-(quinoline-8-sulfonyl)-1H-pyrazolo[4,3-c]pyridin-6-ylamino]-1,3-dihydro-indol-2-one;
(1-piperidin-4-yl-1H-pyrazol-4-yl)-[7-(quinoline-8-sulfonyl)-7H-pyrrolo[2,3-d]pyrimidin-2-yl]-amine;
1-{3-methoxy-4-[7-(quinoline-8-sulfonyl)-7H-pyrrolo[2,3-d]pyrimidin-2-ylamino]-phenyl}-piperidin-4-ol;
[7-(6-methyl-quinoline-8-sulfonyl)-7H-pyrrolo[2,3-d]pyrimidin-2-yl]-(4-piperazin-1-yl-phenyl)-amine;
(2-cyclobutoxy-4-piperazin-1-yl-phenyl)-[7-(quinoline-8-sulfonyl)-7H-pyrrolo[2,3-d]pyrimidin-2-yl]-amine;
2-[7-(quinoline-8-sulfonyl)-7H-pyrrolo[2,3-d]pyrimidin-2-ylamino]-benzonitrile;
(2-methoxy-4-piperazin-1-yl-phenyl)-[7-(6-methyl-quinoline-8-sulfonyl)-7H-pyrrolo[2,3-d]pyrimidin-2-yl]-amine;
(5-(piperazin-1-yl)-2-((1-(quinolin-8-ylsulfonyl)-1H-pyrrolo[3,2-c]pyridin-6-yl)amino)phenyl)methanol;
(2-cyclobutoxy-4-piperazin-1-yl-phenyl)-[1-(quinoline-8-sulfonyl)-1H-pyrrolo[3,2-c]pyridin-6-yl]-amine;
(5-(piperazin-1-yl)-2-((7-(quinolin-8-ylsulfonyl)-7H-pyrrolo[2,3-d]pyrimidin-2-yl)amino)phenyl)methanol;
N-{3-[6-(3-methoxy-4-piperazin-1-yl-phenylamino)-pyrrolo[3,2-c]pyridin-1-ylmethyl]-pyridin-2-yl}-N-methyl-methanesulfonamide;
(2-methoxy-4-piperazin-1-yl-phenyl)-[7-(2-methyl-quinoline-8-sulfonyl)-7H-pyrrolo[2,3-d]pyrimidin-2-yl]-amine;
N-{3-[6-(2-methoxy-4-piperazin-1-yl-phenylamino)-pyrrolo[3,2-c]pyridin-1-ylmethyl]-pyridin-2-yl}-N-methyl-methanesulfonamide;
[7-(2-methyl-quinoline-8-sulfonyl)-7H-pyrrolo[2,3-d]pyrimidin-2-yl]-(4-piperazin-1-yl-phenyl)-amine;
N-methyl-N-{3-[6-(3-methyl-4-piperazin-1-yl-phenylamino)-pyrrolo[3,2-c]pyridin-1-ylmethyl]-pyridin-2-yl}-methanesulfonamide;
[1-(2-methyl-quinoline-8-sulfonyl)-1H-pyrrolo[3,2-c]pyridin-6-yl]-(4-piperazin-1-yl-phenyl)-amine;
(3-fluoro-2-methoxy-4-piperazin-1-yl-phenyl)-[7-(quinoline-8-sulfonyl)-7H-pyrrolo[2,3-d]pyrimidin-2-yl]-amine;
N-(3-{6-[4-(2,5-diaza-bicyclo[2.2.1]hept-2-yl)-phenylamino]-pyrrolo[3,2-c]pyridin-1-ylmethyl}-pyridin-2-yl)-N-methyl-methanesulfonamide;
N-(3-{2-[4-(2,5-diaza-bicyclo[2.2.1]hept-2-yl)-phenylamino]-pyrrolo[2,3-d]pyrimidin-7-ylmethyl}-pyridin-2-yl)-N-methyl-methanesulfonamide;
cyclobutanecarboxylic acid {2-methoxy-4-[7-(quinoline-8-sulfonyl)-7H-pyrrolo[2,3-d]pyrimidin-2-ylamino]-phenyl}-amide;
N-(3-methyl-4-morpholinophenyl)-1-(quinolin-8-ylsulfonyl)-1H-pyrazolo[4,3-c]pyridin-6-amine;
N-(4-((1-(quinolin-8-ylsulfonyl)-1H-pyrazolo[4,3-c]pyridin-6-yl)amino)phenyl)cyclopropanecarboxamide;
N-(2-methoxy-4-((7-(quinolin-8-ylsulfonyl)-7H-pyrrolo[2,3-d]pyrimidin-2-yl)amino)phenyl)cyclopropanecarboxamide;
N-(3-methyl-4-(4-methylpiperazin-1-yl)phenyl)-1-(quinolin-8-ylsulfonyl)-1H-pyrazolo[4,3-c]pyridin-6-amine;
(4-piperazin-1-yl-phenyl)-(7-quinolin-8-ylmethyl-7H-pyrrolo[2,3-d]pyrimidin-2-yl)-amine;
N-(4-((1-(quinolin-8-ylsulfonyl)-1H-pyrazolo[3,4-d]pyrimidin-6-yl)amino)phenyl)cyclopropanecarboxamide;
N-{3-[5,5-dimethyl-6-oxo-2-(4-piperazin-1-yl-phenylamino)-5,6-dihydro-pyrrolo[2,3-d]pyrimidin-7-ylmethyl]-pyridin-2-yl}-N-methyl-methanesulfonamide;
benzothiazol-6-yl-[7-(quinoline-8-sulfonyl)-7H-pyrrolo[2,3-d]pyrimidin-2-yl]-amine;
(2-methoxy-4-piperazin-1-yl-phenyl)-[1-(2-methyl-quinoline-8-sulfonyl)-1H-pyrrolo[3,2-c]pyridin-6-yl]-amine;
[4-(2,5-diaza-bicyclo[2.2.1]hept-2-yl)-phenyl]-[1-(quinoline-8-sulfonyl)-1H-pyrazolo[4,3-c]pyridin-6-yl]-amine;
N-(3-((6-((4-(4-hydroxypiperidin-1-yl)phenyl)amino)-1H-pyrrolo[3,2-c]pyridin-1-yl)methyl)pyridin-2-yl)-N-methylmethanesulfonamide;
N-methyl-N-(2-((6-((4-morpholinophenyl)amino)-1H-pyrrolo[3,2-c]pyridin-1-yl)methyl)phenyl)methanesulfonamide;
2-(4-{3-methoxy-4-[1-(quinoline-8-sulfonyl)-1H-pyrrolo[3,2-c]pyridin-6-ylamino]-phenyl}-piperazin-1-yl)-ethanol;
2-(4-(3-methoxy-4-((1-(quinolin-8-ylsulfonyl)-1H-pyrazolo[4,3-c]pyridin-6-yl)amino)phenyl)piperazin-1-yl)ethanol;
(4-morpholin-4-yl-phenyl)-[7-(quinoline-8-sulfonyl)-7H-pyrrolo[2,3-d]pyrimidin-2-yl]-amine;
2-[6-(3-methyl-4-piperazin-1-yl-phenylamino)-pyrrolo[3,2-c]pyridine-1-sulfonyl]-benzonitrile;
2-[6-(3-methoxy-4-piperazin-1-yl-phenylamino)-pyrrolo[3,2-c]pyridine-1-sulfonyl]-benzonitrile;
[7-(quinoline-8-sulfonyl)-7H-pyrrolo[2,3-d]pyrimidin-2-yl]-(3,4,5-trimethoxy-phenyl)-amine;
N-[3-(6-{4-[4-(2-hydroxy-ethyl)-piperazin-1-yl]-phenylamino}-pyrrolo[3,2-c]pyridin-1-ylmethyl)-pyridin-2-yl]-N-methyl-methanesulfonamide;
N-(3-{6-[4-(4-hydroxy-piperidin-1-yl)-phenylamino]-pyrazolo[4,3-c]pyridin-1-ylmethyl}-pyridin-2-yl)-N-methyl-methanesulfonamide;
5-((7-(quinolin-8-ylsulfonyl)-7H-pyrrolo[2,3-d]pyrimidin-2-yl)amino)-1H-benzo[d]imidazol-2(3H)-one;
1-{3-methoxy-4-[1-(quinoline-8-sulfonyl)-1H-pyrazolo[4,3-c]pyridin-6-ylamino]-phenyl}-piperidin-4-ol;
N-methyl-N-{3-[6-(4-morpholin-4-yl-phenylamino)-pyrrolo[3,2-c]pyridin-1-ylmethyl]-pyridin-2-yl}-methanesulfonamide;
7-((4-methylquinolin-8-yl)sulfonyl)-N-(4-(piperazin-1-yl)phenyl)-7H-pyrrolo[2,3-d]pyrimidin-2-amine;
5,5-dimethyl-N-(4-morpholinophenyl)-7-(quinolin-8-ylsulfonyl)-6,7-dihydro-5H-pyrrolo[2,3-d]pyrimidin-2-amine;
1-{3-methoxy-4-[1-(quinoline-8-sulfonyl)-1H-pyrrolo[3,2-c]pyridin-6-ylamino]-phenyl}-piperidin-4-ol;
(2-methoxy-4-morpholin-4-yl-phenyl)-[1-(quinoline-8-sulfonyl)-1H-pyrrolo[3,2-c]pyridin-6-yl]-amine;
1-{4-[7-(4-methyl-quinoline-8-sulfonyl)-7H-pyrrolo[2,3-d]pyrimidin-2-ylamino]-phenyl}-piperidin-4-ol;
[5,5-dimethyl-7-(quinoline-8-sulfonyl)-6,7-dihydro-5H-pyrrolo[2,3-d]pyrimidin-2-yl]-(4-piperazin-1-yl-phenyl)-amine;
N-(3-((2-((4-(4-hydroxypiperidin-1-yl)phenyl)amino)-6-oxo-5H-pyrrolo[2,3-d]pyrimidin-7 (6H)-yl)methyl)pyridin-2-yl)-N-methylmethanesulfonamide;
N-(3-hethyl-4-morpholinophenyl)-1-(quinolin-8-ylsulfonyl)-1H-pyrazolo[3,4-d]pyrimidin-6-amine;
1-(2-methyl-4-((1-(quinolin-8-ylsulfonyl)-1H-pyrazolo[3,4-d]pyrimidin-6-yl)amino)phenyl)piperidin-4-ol;
[2-methoxy-4-(4-methyl-piperazin-1-yl)-phenyl]-[1-(quinoline-8-sulfonyl)-1H-pyrrolo[3,2-c]pyridin-6-yl]-amine;
N-methyl-N-(3-((2-((4-(6-oxohexahydropyrrolo[1,2-a]pyrazin-2(1H)-yl)phenyl)amino)-7H-pyrrolo[2,3-d]pyrimidin-7-yl)methyl)pyridin-2-yl)methanesulfonamide;
N-(4-morpholinophenyl)-1-(quinolin-8-ylsulfonyl)-1H-pyrazolo[3,4-d]pyrimidin-6-amine;
[4-(5-methyl-2,5-diaza-bicyclo[2.2.1]hept-2-yl)-phenyl]-[7-(quinoline-8-sulfonyl)-7H-pyrrolo[2,3-d]pyrimidin-2-yl]-amine;
1-(4-((1-(quinolin-8-ylsulfonyl)-1H-pyrazolo[3,4-d]pyrimidin-6-yl)amino)phenyl)piperidin-4-ol;
N-(4-(4-methoxypiperidin-1-yl)phenyl)-7-(quinolin-8-ylsulfonyl)-7H-pyrrolo[2,3-d]pyrimidin-2-amine;
2-((6-((4-(4-(2-hydroxyethyl)piperazin-1-yl)phenyl)amino)-1H-pyrrolo[3,2-c]pyridin-1-yl)sulfonyl)benzonitrile;
2-{6-[4-(4-hydroxy-piperidin-1-yl)-phenylamino]-pyrrolo[3,2-c]pyridine-1-sulfonyl}-benzonitrile;
N-(3-((2-((4-(4-hydroxypiperidin-1-yl)phenyl)amino)-7H-pyrrolo[2,3-d]pyrimidin-7-yl)methyl)pyridin-2-yl)-N-methylmethanesulfonamide;
2-(4-(4-((1-(quinolin-8-ylsulfonyl)-1H-pyrazolo[3,4-d]pyrimidin-6-yl)amino)phenyl)piperazin-1-yl)ethanol;
2-{4-[7-(quinoline-8-sulfonyl)-7H-pyrrolo[2,3-d]pyrimidin-2-ylamino]-phenyl}-hexahydro-pyrrolo[1,2-a]pyrazin-6-one;
N-(2-chloro-4-(piperazin-1-yl)phenyl)-7-(quinolin-8-ylsulfonyl)-7H-pyrrolo[2,3-d]pyrimidin-2-amine;
2-(6-{4-[4-(2-hydroxy-ethyl)-piperazin-1-yl]-phenylamino}-pyrazolo[4,3-c]pyridine-1-sulfonyl)-benzonitrile;
N-{2-[2-(3-methoxy-4-piperazin-1-yl-phenylamino)-pyrrolo[2,3-d]pyrimidin-7-ylmethyl]-phenyl}-N-methyl-methanesulfonamide;
N-[2-(2-{4-[4-(2-hydroxy-ethyl)-piperazin-1-yl]-3-methoxy-phenylamino}-pyrrolo[2,3-d]pyrimidin-7-ylmethyl)-phenyl]-N-methyl-methanesulfonamide;
N-(2-{2-[4-(4-hydroxy-piperidin-1-yl)-3-methoxy-phenylamino]-pyrrolo[2,3-d]pyrimidin-7-ylmethyl}-phenyl)-N-methyl-methanesulfonamide;
N-[2-(2-{4-[4-(2-hydroxy-ethyl)-piperazin-1-yl]-phenylamino}-pyrrolo[2,3-d]pyrimidin-7-ylmethyl)-phenyl]-N-methyl-methanesulfonamide;
N-(2-{2-[2-methoxy-4-(4-methyl-piperazin-1-yl)-phenylamino]-pyrrolo[2,3-d]pyrimidin-7-ylmethyl}-phenyl)-N-methyl-methanesulfonamide;
(1-(4-((7-(quinolin-8-ylsulfonyl)-7H-pyrrolo[2,3-d]pyrimidin-2-yl)amino)phenyl)piperidin-3-yl)methanol;
N-(2-((2-((2-methoxy-4-(piperazin-1-yl)phenyl)amino)-7H-pyrrolo[2,3-d]pyrimidin-7-yl)methyl)phenyl)-N-methylmethanesulfonamide;
[3-methoxy-4-(4-methyl-piperazin-1-yl)-phenyl]-[7-(quinoline-8-sulfonyl)-7H-pyrrolo[2,3-d]pyrimidin-2-yl]-amine;
N-methyl-N-(2-{2-[4-(4-methyl-piperazin-1-yl)-phenylamino]-pyrrolo[2,3-d]pyrimidin-7-ylmethyl}-phenyl)-methanesulfonamide;
N-(2-{2-[3-methoxy-4-(4-methyl-piperazin-1-yl)-phenylamino]-pyrrolo[2,3-d]pyrimidin-7-ylmethyl}-phenyl)-N-methyl-methanesulfonamide;
[2-methoxy-4-(4-methoxy-piperidin-1-yl)-phenyl]-[1-(quinoline-8-sulfonyl)-1H-pyrrolo[3,2-c]pyridin-6-yl]-amine;
2-(4-{2-methoxy-4-[7-(quinoline-8-sulfonyl)-7H-pyrrolo[2,3-d]pyrimidin-2-ylamino]-phenyl}-piperazin-1-yl)-ethanol;
(3-methoxy-4-morpholin-4-yl-phenyl)-[7-(quinoline-8-sulfonyl)-7H-pyrrolo[2,3-d]pyrimidin-2-yl]-amine;
N-methyl-N-{3-[2-(4-piperazin-1-yl-phenylamino)-5,6-dihydro-pyrrolo[2,3-d]pyrimidin-7-ylmethyl]-pyridin-2-yl}-methanesulfonamide;
N-(2-{2-[4-(4-hydroxy-piperidin-1-yl)-phenylamino]-pyrrolo[2,3-d]pyrimidin-7-ylmethyl}-phenyl)-N-methyl-methanesulfonamide;
N-{2-[2-(1,3-dioxo-2,3-dihydro-1H-isoindol-5-ylamino)-pyrrolo[2,3-d]pyrimidin-7-ylmethyl]-phenyl}-N-methyl-methanesulfonamide;
N-methyl-N-(2-{2-[4-(6-oxo-hexahydro-pyrrolo[1,2-a]pyrazin-2-yl)-phenylamino]-pyrrolo[2,3-d]pyrimidin-7-ylmethyl}-phenyl)-methanesulfonamide;
N-{3-[5,5-dimethyl-2-(4-piperazin-1-yl-phenylamino)-5,6-dihydro-pyrrolo[2,3-d]pyrimidin-7-ylmethyl]-pyridin-2-yl}-N-methyl-methanesulfonamide;
N-{2-[2-(3-fluoro-4-piperazin-1-yl-phenylamino)-pyrrolo[2,3-d]pyrimidin-7-ylmethyl]-phenyl}-N-methyl-methanesulfonamide;
[3-methoxy-4-(5-methyl-2,5-diaza-bicyclo[2.2.1]hept-2-yl)-phenyl]-[7-(quinoline-8-sulfonyl)-7H-pyrrolo[2,3-d]pyrimidin-2-yl]-amine;
(1-{4-[7-(quinoline-8-sulfonyl)-7H-pyrrolo[2,3-d]pyrimidin-2-ylamino]-phenyl}-piperidin-4-yl)-methanol;
acetic acid 1-(4-{7-[2-(methanesulfonyl-methyl-amino)-benzyl]-7H-pyrrolo[2,3-d]pyrimidin-2-ylamino}-3-methoxy-phenyl)-piperidin-4-yl ester;
1-(5-{4-[7-(quinoline-8-sulfonyl)-7H-pyrrolo[2,3-d]pyrimidin-2-ylamino]-phenyl}-2,5-diaza-bicyclo[2.2.1]hept-2-yl)-ethanone;
N-(4-{7-[2-(methanesulfonyl-methyl-amino)-benzyl]-7H-pyrrolo[2,3-d]pyrimidin-2-ylamino}-phenyl)-cetamide;
{2-methoxy-4-[4-(2-methoxy-ethyl)-piperazin-1-yl]-phenyl}-[1-(quinoline-8-sulfonyl)-1H-pyrrolo[3,2-c]pyridin-6-yl]-amine;
[5,5-dimethyl-7-(quinoline-8-sulfonyl)-6,7-dihydro-5H-pyrrolo[2,3-d]pyrimidin-2-yl]-[4-(4-methoxy-piperidin-1-yl)-phenyl]-amine;
[5,5-dimethyl-7-(quinoline-8-sulfonyl)-6,7-dihydro-5H-pyrrolo[2,3-d]pyrimidin-2-yl]-(2-methoxy-4-piperazin-1-yl-phenyl)-amine;
N-methyl-N-{2-[2-(3-methyl-4-piperazin-1-yl-phenylamino)-pyrrolo[2,3-d]pyrimidin-7-ylmethyl]-phenyl}-methanesulfonamide;
(R)—N-methyl-N-(2-((2-((4-(3-methylpiperazin-1-yl)phenyl)amino)-7H-pyrrolo[2,3-d]pyrimidin-7-yl)methyl)phenyl)methanesulfonamide;
1-((S)-2-methyl-4-{4-[7-(quinoline-8-sulfonyl)-7H-pyrrolo[2,3-d]pyrimidin-2-ylamino]-phenyl}-piperazin-1-yl)-ethanone;
cyclopropanecarboxylic acid (4-{7-[2-(methanesulfonyl-methyl-amino)-benzyl]-7H-pyrrolo[2,3-d]pyrimidin-2-ylamino}-phenyl)-amide;
acetic acid 2-(4-{3-methoxy-4-[1-(quinoline-8-sulfonyl)-1H-pyrrolo[3,2-c]pyridin-6-ylamino]-phenyl}-piperazin-1-yl)-ethyl ester;
2-hydroxy-N-(4-{7-[2-(methanesulfonyl-methyl-amino)-benzyl]-7H-pyrrolo[2,3-d]pyrimidin-2-ylamino}-phenyl)-2-methyl-propionamide;
acetic acid 1-{4-[5,5-dimethyl-7-(quinoline-8-sulfonyl)-6,7-dihydro-5H-pyrrolo[2,3-d]pyrimidin-2-ylamino]-phenyl}-piperidin-4-yl ester;
acetic acid 1-{4-[7-(quinoline-8-sulfonyl)-7H-pyrrolo[2,3-d]pyrimidin-2-ylamino]-phenyl}-piperidin-4-yl ester;
N-{3-[6-hydroxy-5,5-dimethyl-2-(4-piperazin-1-yl-phenylamino)-5,6-dihydro-pyrrolo[2,3-d]pyrimidin-7-ylmethyl]-pyridin-2-yl}-N-methyl-methanesulfonamide;
N-{2-[2-(3-fluoro-2-methoxy-4-piperazin-1-yl-phenylamino)-pyrrolo[2,3-d]pyrimidin-7-ylmethyl]-phenyl}-N-methyl-methanesulfonamide;
N-methyl-N-{2-[2-(2-oxo-2,3-dihydro-benzooxazol-6-ylamino)-pyrrolo[2,3-d]pyrimidin-7-ylmethyl]-phenyl}-methanesulfonamide;
N-(2-{2-[4-((R)-4-acetyl-3-methyl-piperazin-1-yl)-phenylamino]-pyrrolo[2,3-d]pyrimidin-7-ylmethyl}-phenyl)-N-methyl-methanesulfonamide;
N-methyl-N-{2-[2-(4-piperidin-4-yl-phenylamino)-pyrrolo[2,3-d]pyrimidin-7-ylmethyl]-phenyl}-methanesulfonamide;
{2-methoxy-4-[4-(2-methoxy-ethyl)-piperazin-1-yl]-phenyl}-[1-(quinoline-8-sulfonyl)-1H-pyrazolo[4,3-c]pyridin-6-yl]-amine;
2-[5,5-dimethyl-2-(4-piperazin-1-yl-phenylamino)-5,6-dihydro-pyrrolo[2,3-d]pyrimidine-7-sulfonyl]-benzonitrile;
[4-(4-dimethylamino-piperidin-1-yl)-phenyl]-[7-(quinoline-8-sulfonyl)-7H-pyrrolo[2,3-d]pyrimidin-2-yl]-amine;
N-(2-{2-[4-(4-amino-piperidin-1-yl)-phenylamino]-pyrrolo[2,3-d]pyrimidin-7-ylmethyl}-phenyl)-N-methyl-methanesulfonamide;
[7-(2-difluoromethoxy-benzenesulfonyl)-5,5-dimethyl-6,7-dihydro-5H-pyrrolo[2,3-d]pyrimidin-2-yl]-(4-piperazin-1-yl-phenyl)-amine;
N-(2-{2-[4-(4-acetyl-piperazin-1-yl)-phenylamino]-pyrrolo[2,3-d]pyrimidin-7-ylmethyl}-phenyl)-N-methyl-methanesulfonamide;
2-{5,5-dimethyl-2-[4-((R)-3-methyl-piperazin-1-yl)-phenylamino]-5,6-dihydro-pyrrolo[2,3-d]pyrimidine-7-sulfonyl}-benzonitrile;
N-methyl-N-(2-{2-[4-((S)-3-methyl-piperazin-1-yl)-phenylamino]-pyrrolo[2,3-d]pyrimidin-7-ylmethyl}-phenyl)-methanesulfonamide;
N-(2-{2-[4-(4-dimethylamino-piperidin-1-yl)-phenylamino]-pyrrolo[2,3-d]pyrimidin-7-ylmethyl}-phenyl)-N-methyl-methanesulfonamide;
2-[5,5-dimethyl-2-(3-methyl-4-piperazin-1-yl-phenylamino)-5,6-dihydro-pyrrolo[2,3-d]pyrimidine-7-sulfonyl]-benzonitrile;
[5,5-dimethyl-7-(quinoline-8-sulfonyl)-6,7-dihydro-5H-pyrrolo[2,3-d]pyrimidin-2-yl]-[4-((R)-3-methyl-piperazin-1-yl)-phenyl]-amine;
N-methyl-N-(3-{2-[4-((S)-3-methyl-piperazin-1-yl)-phenylamino]-pyrrolo[2,3-d]pyrimidin-7-ylmethyl}-pyridin-2-yl)-methanesulfonamide;
2-{6-[4-((S)-3-methyl-piperazin-1-yl)-phenylamino]-pyrrolo[3,2-c]pyridine-1-sulfonyl}-benzonitrile;
[5,5-dimethyl-7-(quinoline-8-sulfonyl)-6,7-dihydro-5H-pyrrolo[2,3-d]pyrimidin-2-yl]-[4-((S)-3-methyl-piperazin-1-yl)-phenyl]-amine;
N-methyl-2-(2-{2-[4-(4-methyl-piperazin-1-yl)-phenylamino]-pyrrolo[2,3-d]pyrimidin-7-ylmethyl}-phenyl)-acetamide;
N-methyl-N-(3-{2-[4-((R)-3-methyl-piperazin-1-yl)-phenylamino]-pyrrolo[2,3-d]pyrimidin-7-ylmethyl}-pyridin-2-yl)-methanesulfonamide;
N-(3-{2-[4-(4-ethyl-piperazin-1-yl)-phenylamino]-pyrrolo[2,3-d]pyrimidin-7-ylmethyl}-pyridin-2-yl)-N-methyl-methanesulfonamide;
2-{6-[4-((S)-2-methyl-piperazin-1-yl)-phenylamino]-pyrrolo[3,2-c]pyridine-1-sulfonyl}-benzonitrile;
[4-((S)-2-methyl-piperazin-1-yl)-phenyl]-[1-(quinoline-8-sulfonyl)-1H-pyrrolo[3,2-c]pyridin-6-yl]-amine;
2-{6-[4-((R)-2-methyl-piperazin-1-yl)-phenylamino]-pyrrolo[3,2-c]pyridine-1-sulfonyl}-benzonitrile;
[4-((R)-2-methyl-piperazin-1-yl)-phenyl]-[1-(quinoline-8-sulfonyl)-1H-pyrrolo[3,2-c]pyridin-6-yl]-amine;
(S)—N-methyl-N-(2-((2-((4-(2-methylpiperazin-1-yl)phenyl)amino)-7H-pyrrolo[2,3-d]pyrimidin-7-yl)methyl)phenyl)methanesulfonamide;
(R)—N-methyl-N-(2-((2-((4-(2-methylpiperazin-1-yl)phenyl)amino)-7H-pyrrolo[2,3-d]pyrimidin-7-yl)methyl)phenyl)methanesulfonamide;
N-methyl-N-(3-{2-[4-((R)-2-methyl-piperazin-1-yl)-phenylamino]-pyrrolo[2,3-d]pyrimidin-7-ylmethyl}-pyridin-2-yl)-methanesulfonamide;
N-(2-{2-[4-(4-isopropyl-piperazin-1-yl)-phenylamino]-pyrrolo[2,3-d]pyrimidin-7-ylmethyl}-phenyl)-N-methyl-methanesulfonamide;
2-{6-[4-(4-dimethylamino-piperidin-1-yl)-phenylamino]-pyrrolo[3,2-c]pyridine-1-sulfonyl}-benzonitrile;
N-(2-{2-[4-((R)-3,4-dimethyl-piperazin-1-yl)-phenylamino]-pyrrolo[2,3-d]pyrimidin-7-ylmethyl}-phenyl)-N-methyl-methanesulfonamide;
N-(3-{2-[4-((S)-3,4-dimethyl-piperazin-1-yl)-phenylamino]-pyrrolo[2,3-d]pyrimidin-7-ylmethyl}-pyridin-2-yl)-N-methyl-methanesulfonamide;
N-(2-{2-[4-((R)-2,4-dimethyl-piperazin-1-yl)-phenylamino]-pyrrolo[2,3-d]pyrimidin-7-ylmethyl}-phenyl)-N-methyl-methanesulfonamide;
N-(3-{2-[4-((R)-2,4-dimethyl-piperazin-1-yl)-phenylamino]-pyrrolo[2,3-d]pyrimidin-7-ylmethyl}-pyridin-2-yl)-N-methyl-methanesulfonamide;
[4-((R)-2-methyl-piperazin-1-yl)-phenyl]-[7-(quinoline-8-sulfonyl)-7H-pyrrolo[2,3-d]pyrimidin-2-yl]-amine;
N-(2-{2-[4-((S)-3,4-dimethyl-piperazin-1-yl)-phenylamino]-pyrrolo[2,3-d]pyrimidin-7-ylmethyl}-phenyl)-N-methyl-methanesulfonamide;
2-{6-[3-methoxy-4-(4-methyl-piperazin-1-yl)-phenylamino]-pyrrolo[3,2-c]pyridine-1-sulfonyl}-benzonitrile;
2-{6-[2-methoxy-4-(4-methyl-piperazin-1-yl)-phenylamino]-pyrrolo[3,2-c]pyridine-1-sulfonyl}-benzonitrile;
N-(4-(piperazin-1-yl)phenyl)-7′-(quinolin-8-ylsulfonyl)-6′,7′-dihydrospiro [cyclopropane-1,5′-pyrrolo[2,3-d]pyrimidin]-2′-amine;
N-(2-{2-[4-((S)-2,4-dimethyl-piperazin-1-yl)-phenylamino]-pyrrolo[2,3-d]pyrimidin-7-ylmethyl}-phenyl)-N-methyl-methanesulfonamide;
N-(2-{2-[4-(hexahydro-pyrrolo[1,2-a]pyrazin-2-yl)-phenylamino]-pyrrolo[2,3-d]pyrimidin-7-ylmethyl}-phenyl)-N-methyl-methanesulfonamide;
N-(3-{2-[4-((S)-2,4-dimethyl-piperazin-1-yl)-phenylamino]-pyrrolo[2,3-d]pyrimidin-7-ylmethyl}-pyridin-2-yl)-N-methyl-methanesulfonamide;
[5,5-dimethyl-7-(quinoline-8-sulfonyl)-6,7-dihydro-5H-pyrrolo[2,3-d]pyrimidin-2-yl]-[4-(4-methyl-piperazin-1-yl)-phenyl]-amine;
N-(4-(4-methylpiperazin-1-yl)phenyl)-7′-(quinolin-8-ylsulfonyl)-6′,7′-dihydrospiro[cyclopropane-1,5′-pyrrolo[2,3-d]pyrimidin]-2′-amine;
[4-(4-dimethylamino-piperidin-1-yl)-phenyl]-[5,5-dimethyl-7-(quinoline-8-sulfonyl)-6,7-dihydro-5H-pyrrolo[2,3-d]pyrimidin-2-yl]-amine;
N-(3-((2-((3-fluoro-4-(morpholine-4-carbonyl)phenyl)amino)-7H-pyrrolo[2,3-d]pyrimidin-7-yl)methyl)pyridin-2-yl)-N-methylmethanesulfonamide;
N-(2-{2-[3-fluoro-4-(morpholine-4-carbonyl)-phenylamino]-pyrrolo[2,3-d]pyrimidin-7-ylmethyl}-phenyl)-N-methyl-methanesulfonamide;
N-(2-{2-[2-methoxy-4-(morpholine-4-carbonyl)-phenylamino]-pyrrolo[2,3-d]pyrimidin-7-ylmethyl}-phenyl)-N-methyl-methanesulfonamide;
[5,5-dimethyl-7-(quinoline-8-sulfonyl)-6,7-dihydro-5H-pyrrolo[2,3-d]pyrimidin-2-yl]-[2-methoxy-4-(4-methyl-piperazin-1-yl)-phenyl]-amine;
2-{6-[4-(4-methyl-[1,4]diazepan-1-yl)-phenylamino]-pyrrolo[3,2-c]pyridine-1-sulfonyl}-benzonitrile;
2-{6-[4-(4-methyl-piperazin-1-yl)-phenylamino]-pyrrolo[3,2-c]pyridine-1-sulfonyl}-benzonitrile;
2-fluoro-N-(2-hydroxy-2-methyl-propyl)-4-{7-[2-(methanesulfonyl-methyl-amino)-benzyl]-7H-pyrrolo[2,3-d]pyrimidin-2-ylamino}-benzamide;
[7-(2-difluoromethoxy-phenyl)-7H-pyrrolo[2,3-d]pyrimidin-2-yl]-[4-(4-methyl-piperazin-1-yl)-phenyl]-amine;
7′-((2,5-dichlorophenyl)sulfonyl)-N-(4-(piperazin-1-yl)phenyl)-6′,7′-dihydrospiro[cyclopropane-1,5′-pyrrolo[2,3-d]pyrimidin]-2′-amine;
5-[5,5-dimethyl-7-(quinoline-8-sulfonyl)-6,7-dihydro-5H-pyrrolo[2,3-d]pyrimidin-2-ylamino]-1,3-dihydro-indol-2-one;
N-(2-{2-[5-fluoro-2-methoxy-4-(morpholine-4-carbonyl)-phenylamino]-pyrrolo[2,3-d]pyrimidin-7-ylmethyl}-phenyl)-N-methyl-methanesulfonamide;
N-methyl-N-{2-[2-(2-oxo-2,3-dihydro-1H-indol-5-ylamino)-pyrrolo[2,3-d]pyrimidin-7-ylmethyl]-phenyl}-methanesulfonamide;
2-[6-(2-oxo-2,3-dihydro-1H-indol-5-ylamino)-pyrrolo[3,2-c]pyridine-1-sulfonyl]-benzonitrile;
N-(2-{2-[4-(azetidin-3-yloxy)-phenylamino]-pyrrolo[2,3-d]pyrimidin-7-ylmethyl}-phenyl)-N-methyl-methanesulfonamide;
2-fluoro-N-(2-hydroxy-2-methyl-propyl)-4-{7-[2-methanesulfonyl-methyl-amino)-pyridin-3-ylmethyl]-7H-pyrrolo[2,3-d]pyrimidin-2-ylamino}-benzamide;
[5,5-dimethyl-7-(quinoline-8-sulfonyl)-6,7-dihydro-5H-pyrrolo[2,3-d]pyrimidin-2-yl]-(3-methoxy-4-piperazin-1-yl-phenyl)-amine;
N-{2-[2-(4-piperazin-1-yl-phenylamino)-pyrrolo[2,3-d]pyrimidin-7-ylmethyl]-phenyl}-methanesulfonamide;
N-methyl-N-(2-{2-[4-(4-methyl-[1,4]diazepan-1-yl)-phenylamino]-pyrrolo[2,3-d]pyrimidin-7-ylmethyl}-phenyl)-methanesulfonamide;
N-{2-[2-(4-[1,4]diazepan-1-yl-phenylamino)-pyrrolo[2,3-d]pyrimidin-7-ylmethyl]-phenyl}-N-methyl-methanesulfonamide;
N-(3-{2-[4-(3,5-dimethyl-piperazin-1-yl)-phenylamino]-pyrrolo[2,3-d]pyrimidin-7-ylmethyl}-pyridin-2-yl)-N-methyl-methanesulfonamide;
N-(2-{2-[4-(3,5-dimethyl-piperazin-1-yl)-phenylamino]-pyrrolo[2,3-d]pyrimidin-7-ylmethyl}-phenyl)-N-methyl-methanesulfonamide;
[5,5-dimethyl-7-(quinoline-8-sulfonyl)-6,7-dihydro-5H-pyrrolo[2,3-d]pyrimidin-2-yl]-(3-fluoro-4-piperazin-1-yl-phenyl)-amine;
N-(2-hydroxy-ethyl)-N-{2-[2-(4-piperazin-1-yl-phenylamino)-pyrrolo[2,3-d]pyrimidin-7-ylmethyl]-phenyl}-methanesulfonamide;
N-{2-[2-(1,2,3,4,10,10a-hexahydro-pyrazino [1,2-a]indol-8-ylamino)-pyrrolo[2,3-d]pyrimidin-7-ylmethyl]-phenyl}-N-methyl-methanesulfonamide;
N-(2-{2-[4-(2,5-dimethyl-piperazin-1-yl)-phenylamino]-pyrrolo[2,3-d]pyrimidin-7-ylmethyl}-phenyl)-N-methyl-methanesulfonamide;
N-{2-[5,5-dimethyl-2-(4-piperazin-1-yl-phenylamino)-5,6-dihydro-pyrrolo[2,3-d]pyrimidine-7-carbonyl]-phenyl}-N-methyl-methanesulfonamide;
2-{6-[4-(3,5-dimethyl-piperazin-1-yl)-phenylamino]-pyrrolo[3,2-c]pyridine-1-sulfonyl}-benzonitrile;
N-(3-{2-[4-(4-dimethylamino-piperidin-1-yl)-phenylamino]-pyrrolo[2,3-d]pyrimidin-7-ylmethyl}-pyridin-2-yl)-N-methyl-methanesulfonamide;
N-methyl-N-(2-{2-[4-(1-methyl-piperidin-4-yloxy)-phenylamino]-pyrrolo[2,3-d]pyrimidin-7-ylmethyl}-phenyl)-methanesulfonamide;
2-[3,3-dimethyl-6-(4-piperazin-1-yl-phenylamino)-2,3-dihydro-pyrrolo[3,2-c]pyridine-1-sulfonyl]-benzonitrile;
N-methyl-N-{3-[2-(4-piperazin-1-yl-phenylamino)-pyrrolo[2,3-d]pyrimidin-7-yl]-phenyl}-methanesulfonamide;
(5,5-dimethyl-7-pyridin-2-yl-6,7-dihydro-5H-pyrrolo[2,3-d]pyrimidin-2-yl)-(4-piperazin-1-yl-phenyl)-amine;
N-(3-{2-[4-(2-hydroxymethyl-pyrrolidin-1-yl)-phenylamino]-pyrrolo[2,3-d]pyrimidin-7-ylmethyl}-pyridin-2-yl)-N-methyl-methanesulfonamide;
2-{6-[4-((R)-3-methyl-piperazin-1-yl)-phenylamino]-pyrrolo[3,2-c]pyridine-1-sulfonyl}-benzonitrile;
N-{3-[2-(1,2,3,4,10,10a-hexahydro-pyrazino [1,2-a]indol-8-ylamino)-pyrrolo[2,3-d]pyrimidin-7-ylmethyl]-pyridin-2-yl}-N-methyl-methanesulfonamide;
N-methyl-N-(2-((2-((3-oxo-3,4-dihydro-2H-benzo[b][1,4]oxazin-7-yl)amino)-7H-pyrrolo[2,3-d]pyrimidin-7-yl)methyl)phenyl)methanesulfonamide;
N-(3-{2-[4-(3,3-dimethyl-2-oxo-piperazin-1-yl)-phenylamino]-pyrrolo[2,3-d]pyrimidin-7-ylmethyl}-pyridin-2-yl)-N-methyl-methanesulfonamide;
[5,5-dimethyl-7-(thiophene-2-sulfonyl)-6,7-dihydro-5H-pyrrolo[2,3-d]pyrimidin-2-yl]-(4-piperazin-1-yl-phenyl)-amine;
(7-benzenesulfonyl-5,5-dimethyl-6,7-dihydro-5H-pyrrolo[2,3-d]pyrimidin-2-yl)-(4-piperazin-1-yl-phenyl)-amine;
[7-(2-methoxy-benzenesulfonyl)-5,5-dimethyl-6,7-dihydro-5H-pyrrolo[2,3-d]pyrimidin-2-yl]-(4-piperazin-1-yl-phenyl)-amine;
6-[5,5-dimethyl-7-(quinoline-8-sulfonyl)-6,7-dihydro-5H-pyrrolo[2,3-d]pyrimidin-2-ylamino]-3,4-dihydro-1H-quinolin-2-one;
N-{2-[2-(2-oxo-2,3-dihydro-1H-indol-5-ylamino)-pyrrolo[2,3-d]pyrimidin-7-ylmethyl]-phenyl}-methanesulfonamide;
N-(2-{2-[4-((R)-3-methyl-piperazin-1-yl)-phenylamino]-pyrrolo[2,3-d]pyrimidin-7-ylmethyl}-phenyl)-methanesulfonamide;
N-(2-hydroxy-ethyl)-N-(2-{2-[4-((R)-3-methyl-piperazin-1-yl)-phenylamino]-pyrrolo[2,3-d]pyrimidin-7-ylmethyl}-phenyl)-ethanesulfonamide;
N-{3-[2-(4-[1,4]diazepan-1-yl-phenylamino)-pyrrolo[2,3-d]pyrimidin-7-ylmethyl]-pyridin-2-yl}-N-methyl-methanesulfonamide;
N-methyl-N-(2-((2-((4-(2-oxopiperazin-1-yl)phenyl)amino)-7H-pyrrolo[2,3-d]pyrimidin-7-yl)methyl)phenyl)methanesulfonamide;
2-((6-((1,2,3,4,10,10a-hexahydropyrazino[1,2-a]indol-8-yl)amino)-1H-pyrrolo[3,2-c]pyridin-1-yl)sulfonyl)benzonitrile;
N-(2-{2-[4-(3,3-dimethyl-2-oxo-piperazin-1-yl)-phenylamino]-pyrrolo[2,3-d]pyrimidin-7-ylmethyl}-phenyl)-N-methyl-methanesulfonamide;
N-(3-{2-[3-fluoro-4-((S)-3-methyl-piperazin-1-yl)-phenylamino]-pyrrolo[2,3-d]pyrimidin-7-ylmethyl}-pyridin-2-yl)-N-methyl-methanesulfonamide;
[5,5-dimethyl-7-(quinoline-8-sulfonyl)-6,7-dihydro-5H-pyrrolo[2,3-d]pyrimidin-2-yl]-[3-fluoro-4-((S)-3-methyl-piperazin-1-yl)-phenyl]-amine;
N-(2-{2-[3-fluoro-4-((S)-3-methyl-piperazin-1-yl)-phenylamino]-pyrrolo[2,3-d]pyrimidin-7-ylmethyl}-phenyl)-N-methyl-methanesulfonamide;
N-(2-amino-ethyl)-N-{2-[2-(4-piperazin-1-yl-phenylamino)-pyrrolo[2,3-d]pyrimidin-7-ylmethyl]-phenyl}-methanesulfonamide;
N-(3-{2-[4-(4-hydroxymethyl-piperidin-1-yl)-phenylamino]-pyrrolo[2,3-d]pyrimidin-7-ylmethyl}-pyridin-2-yl)-N-methyl-methanesulfonamide;
N-methyl-N-{3-[2-(1-methyl-2-oxo-2,3-dihydro-1H-indol-5-ylamino)-pyrrolo[2,3-d]pyrimidin-7-ylmethyl]-pyridin-2-yl}-methanesulfonamide;
N-(3-{2-[3-fluoro-4-((R)-3-methyl-piperazin-1-yl)-phenylamino]-pyrrolo[2,3-d]pyrimidin-7-ylmethyl}-pyridin-2-yl)-N-methyl-methanesulfonamide;
N-(2-{2-[3-fluoro-4-((R)-3-methyl-piperazin-1-yl)-phenylamino]-pyrrolo[2,3-d]pyrimidin-7-ylmethyl}-phenyl)-N-methyl-methanesulfonamide;
N-methyl-N-(2-{2-[6-((R)-3-methyl-piperazin-1-yl)-pyridin-3-ylamino]-pyrrolo[2,3-d]pyrimidin-7-ylmethyl}-phenyl)-methanesulfonamide;
[5,5-dimethyl-7-(quinoline-8-sulfonyl)-6,7-dihydro-5H-pyrrolo[2,3-d]pyrimidin-2-yl]-[4-((S)-2-methyl-piperazin-1-yl)-phenyl]-amine;
N-methyl-N-(2-{2-[5-((S)-3-methyl-piperazin-1-yl)-pyridin-2-ylamino]-pyrrolo[2,3-d]pyrimidin-7-ylmethyl}-phenyl)-methanesulfonamide;
N-(2-{2-[4-(4-hydroxymethyl-piperidin-1-yl)-phenylamino]-pyrrolo[2,3-d]pyrimidin-7-ylmethyl}-phenyl)-N-methyl-methanesulfonamide;
N-methyl-N-(2-((2-((4-thiomorpholinophenyl)amino)-7H-pyrrolo[2,3-d]pyrimidin-7-yl)methyl)phenyl)methanesulfonamide;
N-(2-((2-((3-(hydroxymethyl)-4-(piperazin-1-yl)phenyl)amino)-7H-pyrrolo[2,3-d]pyrimidin-7-yl)methyl)phenyl)-N-methylmethanesulfonamide;
N-methyl-N-(3-((2-((4-thiomorpholinophenyl)amin)-7H-pyrrolo[2,3-d]pyrimidin-7-yl)methyl)pyridin-2-yl)methanesulfonamide;
N-(2-((2-((4-(2-(hydroxymethyl)pyrrolidin-1-yl)phenyl)amino)-7H-pyrrolo[2,3-d]pyrimidin-7-yl)methyl)phenyl)-N-methylmethanesulfonamide;
N-(2-((2-((4-(3-(dimethylamino)pyrrolidin-1-yl)phenyl)amino)-7H-pyrrolo[2,3-d]pyrimidin-7-yl)methyl)phenyl)-N-methylmethanesulfonamide;
N-methyl-N-(2-{2-[4-(2-piperazin-1-yl-ethoxy)-phenylamino]-pyrrolo[2,3-d]pyrimidin-7-ylmethyl}-phenyl)-methanesulfonamide;
5,5-Dimethyl-N-(4-(piperidin-4-yloxy)phenyl)-7-(quinolin-8-ylsulfonyl)-6,7-dihydro-5H-pyrrolo[2,3-d]pyrimidin-2-amine;
(S)—N-(3-((2-((3-fluoro-4-(2-methylpiperazin-1-yl)phenyl)amino)-7H-pyrrolo[2,3-d]pyrimidin-7-yl)methyl)pyridin-2-yl)-N-methylmethanesulfonamide;
N-methyl-N-(2-{2-[3-methyl-4-((R)-3-methyl-piperazin-1-yl)-phenylamino]-pyrrolo[2,3-d]pyrimidin-7-ylmethyl}-phenyl)-methanesulfonamide;
N-methyl-N-(2-{2-[4-(2-oxo-2H-pyridin-1-yl)-phenylamino]-pyrrolo[2,3-d]pyrimidin-7-ylmethyl}-phenyl)-methanesulfonamide;
N-(2-{2-[4-(4-dimethylamino-piperidin-1-yl)-phenylamino]-pyrrolo[2,3-d]pyrimidin-7-ylmethyl}-phenyl)-methanesulfonamide;
(S)—N-(2-((2-((3-fluoro-4-(2-methylpiperazin-1-yl)phenyl)amino)-7H-pyrrolo[2,3-d]pyrimidin-7-yl)methyl)phenyl)-N-methylmethanesulfonamide;
N-methyl-N-(3-{2-[4-(2-piperazin-1-yl-ethoxy)-phenylamino]-pyrrolo[2,3-d]pyrimidin-7-ylmethyl}-pyridin-2-yl)-methanesulfonamide;
N-(3-{2-[4-(3-dimethylamino-azetidin-1-yl)-phenylamino]-pyrrolo[2,3-d]pyrimidin-7-ylmethyl}-pyridin-2-yl)-N-methyl-methanesulfonamide;
N-(2-{2-[4-(1,1-dioxo-1λ*6*-thiomorpholin-4-yl)-phenylamino]-pyrrolo[2,3-d]pyrimidin-7-ylmethyl}-phenyl)-N-methyl-methanesulfonamide;
N-methyl-N-(3-{2-[6-((S)-3-methyl-piperazin-1-yl)-pyridin-3-ylamino]-pyrrolo[2,3-d]pyrimidin-7-ylmethyl}-pyridin-2-yl)-methanesulfonamide;
N-{2-[2-(3-methoxy-phenylamino)-pyrrolo[2,3-d]pyrimidin-7-ylmethyl]-phenyl}-N-(2-piperazin-1-yl-ethyl)-methanesulfonamide;
N-methyl-N-(3-{2-[3-methyl-4-((R)-3-methyl-piperazin-1-yl)-phenylamino]-pyrrolo[2,3-d]pyrimidin-7-ylmethyl}-pyridin-2-yl)-methanesulfonamide;
(S)—N-methyl-N-(3-((2-((5-(3-methylpiperazin-1-yl)pyridin-2-yl)amino)-7H-pyrrolo[2,3-d]pyrimidin-7-yl)methyl)pyridin-2-yl)methanesulfonamide;
N-methyl-N-(3-{2-[6-((R)-3-methyl-piperazin-1-yl)-pyridin-3-ylamino]-pyrrolo[2,3-d]pyrimidin-7-ylmethyl}-pyridin-2-yl)-methanesulfonamide;
N-(3-{2-[4-((2R,5S)-2,5-dimethyl-piperazin-1-yl)-phenylamino]-pyrrolo[2,3-d]pyrimidin-7-ylmethyl}-pyridin-2-yl)-N-methyl-methanesulfonamide;
N-(2-{2-[4-(3-dimethylamino-azetidin-1-yl)-phenylamino]-pyrrolo[2,3-d]pyrimidin-7-ylmethyl}-phenyl)-N-methyl-methanesulfonamide;
N-(3-{2-[4-(3-dimethylamino-pyrrolidin-1-yl)-phenylamino]-pyrrolo[2,3-d]pyrimidin-7-ylmethyl}-pyridin-2-yl)-N-methyl-methanesulfonamide;
N-(2-((2-((4-(3-(dimethylamino)pyrrolidin-1-yl)-3-fluorophenyl)amino)-7H-pyrrolo[2,3-d]pyrimidin-7-yl)methyl)phenyl)-N-methylmethanesulfonamide;
N-(3-{2-[4-(3-dimethylamino-pyrrolidin-1-yl)-3-fluoro-phenylamino]-pyrrolo[2,3-d]pyrimidin-7-ylmethyl}-pyridin-2-yl)-N-methyl-methanesulfonamide;
(S)—N-(3-((2-((3-(hydroxymethyl)-4-(2-methylpiperazin-1-yl)phenyl)amino)-7H-pyrrolo[2,3-d]pyrimidin-7-yl)methyl)pyridin-2-yl)-N-methylmethanesulfonamide;
(S)—N-(2-((2-((3-(hydroxymethyl)-4-(2-methylpiperazin-1-yl)phenyl)amino)-7H-pyrrolo[2,3-d]pyrimidin-7-yl)methyl)phenyl)-N-methylmethanesulfonamide;
N-(3-((2-((4-(3,3-dimethylpiperazin-1-yl)phenyl)amino)-7H-pyrrolo[2,3-d]pyrimidin-7-yl)methyl)pyridin-2-yl)-N-methylmethanesulfonamide;
N-{2-[2-(2-oxo-1,2,3,4-tetrahydro-quinolin-6-ylamino)-pyrrolo[2,3-d]pyrimidin-7-ylmethyl]-phenyl}-methanesulfonamide;
N-(2-methoxyethyl)-N-(2-((2-((4-(piperazin-1-yl)phenyl)amino)-7H-pyrrolo[2,3-d]pyrimidin-7-yl)methyl)phenyl)methanesulfonamide;
N-methyl-N-(3-{2-[4-(2-oxo-2H-pyridin-1-yl)-phenylamino]-pyrrolo[2,3-d]pyrimidin-7-ylmethyl}-pyridin-2-yl)-methanesulfonamide;
N-(2-((2-((4-(3-(dimethylamino)piperidin-1-yl)phenyl)amino)-7H-pyrrolo[2,3-d]pyrimidin-7-yl)methyl)phenyl)-N-methylmethanesulfonamide;
N-(3-((2-((4-(3-(dimethylamino)piperidin-1-yl)phenyl)amino)-7H-pyrrolo[2,3-d]pyrimidin-7-yl)methyl)pyridin-2-yl)-N-methylmethanesulfonamide;
cyclopropanesulfonic acid methyl-{3-[2-(4-piperazin-1-yl-phenylamino)-pyrrolo[2,3-d]pyrimidin-7-ylmethyl]-pyridin-2-yl}-amide;
N-(3-((2-((4-(4-(dimethylamino)piperidin-1-yl)phenyl)amino)-7H-pyrrolo[2,3-d]pyrimidin-7-yl)methyl)pyridin-2-yl)-N-methylcyclopropanesulfonamide;
N-(2-{2-[4-(3-hydroxymethyl-piperazin-1-yl)-phenylamino]-pyrrolo[2,3-d]pyrimidin-7-ylmethyl}-phenyl)-N-methyl-methanesulfonamide;
[4-(3-dimethylamino-azetidin-1-yl)-phenyl]-[5,5-dimethyl-7-(quinoline-8-sulfonyl)-6,7-dihydro-5H-pyrrolo[2,3-d]pyrimidin-2-yl]-amine;
(S)—N-methyl-N-(3-((2-((4-(3-methylpiperazin-1-yl)phenyl)amino)-7H-pyrrolo[2,3-d]pyrimidin-7-yl)methyl)pyridin-2-yl)cyclopropanesulfonamide;
N-(3-{2-[4-(3-dimethylamino-azetidin-1-yl)-3-fluoro-phenylamino]-pyrrolo[2,3-d]pyrimidin-7-ylmethyl}-pyridin-2-yl)-N-methyl-methanesulfonamide;
N-(2-((2-((4-(3,3-dimethylpiperazin-1-yl)phenyl)amino)-7H-pyrrolo[2,3-d]pyrimidin-7-yl)methyl)phenyl)-N-methylmethanesulfonamide;
N-(3-((2-((3-(hydroxymethyl)-4-(piperazin-1-yl)phenyl)amino)-7H-pyrrolo[2,3-d]pyrimidin-7-yl)methyl)pyridin-2-yl)-N-methylmethanesulfonamide;
N-(2-{2-[4-(3-dimethylamino-azetidin-1-yl)-3-fluoro-phenylamino]-pyrrolo[2,3-d]pyrimidin-7-ylmethyl}-phenyl)-N-methyl-methanesulfonamide;
N-methyl-N-{3-[2-(4-piperazin-2-yl-phenylamino)-pyrrolo[2,3-d]pyrimidin-7-ylmethyl]-pyridin-2-yl}-methanesulfonamide;
N-methyl-N-{2-[2-(4-piperazin-2-yl-phenylamino)-pyrrolo[2,3-d]pyrimidin-7-ylmethyl]-phenyl}-methanesulfonamide;
[4-(4-dimethylamino-piperidin-1-yl)-phenyl]-[3,3-dimethyl-1-(quinoline-8-sulfonyl)-2,3-dihydro-1H-pyrrolo[3,2-c]pyridin-6-yl]-amine;
N-(3-{2-[4-(3-hydroxymethyl-piperazin-1-yl)-phenylamino]-pyrrolo[2,3-d]pyrimidin-7-ylmethyl}-pyridin-2-yl)-N-methyl-methanesulfonamide;
N-methyl-N-(3-{2-[4-(2-morpholin-4-yl-ethoxy)-phenylamino]-pyrrolo[2,3-d]pyrimidin-7-ylmethyl}-pyridin-2-yl)-methanesulfonamide;
2-[6-(4-piperazin-2-yl-phenylamino)-pyrrolo[3,2-c]pyridine-1-sulfonyl]-benzonitrile;
N-(3-{2-[1-(2-hydroxy-ethyl)-2-oxo-2,3-dihydro-1H-indol-5-ylamino]-pyrrolo[2,3-d]pyrimidin-7-ylmethyl}-pyridin-2-yl)-N-methyl-methanesulfonamide;
N-methyl-N-(3-{6-[4-((S)-3-methyl-piperazin-1-yl)-phenylamino]-pyrrolo[3,2-c]pyridin-1-ylmethyl}-pyridin-2-yl)-methanesulfonamide;
[4-(3-dimethylamino-piperidin-1-yl)-phenyl]-[5,5-dimethyl-7-(quinoline-8-sulfonyl)-6,7-dihydro-5H-pyrrolo[2,3-d]pyrimidin-2-yl]-amine;
N-{2-[2-(3-oxo-3,4-dihydro-2H-benzo[1,4]oxazin-7-ylamino)-pyrrolo[2,3-d]pyrimidin-7-ylmethyl]-phenyl}-methanesulfonamide;
[4-(2,5-diaza-bicyclo[2.2.1]hept-2-yl)-phenyl]-[5,5-dimethyl-7-(quinoline-8-sulfonyl)-6,7-dihydro-5H-pyrrolo[2,3-d]pyrimidin-2-yl]-amine;
N-(3-{2-[3-methoxy-4-((S)-3-methyl-piperazin-1-yl)-phenylamino]-pyrrolo[2,3-d]pyrimidin-7-ylmethyl}-pyridin-2-yl)-N-methyl-methanesulfonamide;
[5,5-dimethyl-7-(quinoline-8-sulfonyl)-6,7-dihydro-5H-pyrrolo[2,3-d]pyrimidin-2-yl]-[4-(4-methylamino-piperidin-1-yl)-phenyl]-amine;
[5,5-dimethyl-7-(quinoline-8-sulfonyl)-6,7-dihydro-5H-pyrrolo[2,3-d]pyrimidin-2-yl]-(4-piperazin-2-yl-phenyl)-amine;
2-{6-[4-(3-dimethylamino-azetidin-1-yl)-phenylamino]-pyrrolo[3,2-c]pyridine-1-sulfonyl}-benzonitrile;
N-[3-(2-{4-[4-((S)-2-hydroxy-propyl)-piperazin-1-yl]-phenylamino}-pyrrolo[2,3-d]pyrimidin-7-ylmethyl)-pyridin-2-yl]-N-methyl-methanesulfonamide;
(1-{4-[5,5-dimethyl-7-(quinoline-8-sulfonyl)-6,7-dihydro-5H-pyrrolo[2,3-d]pyrimidin-2-ylamino]-phenyl}-piperidin-4-yl)-methanol;
[4-(3-dimethylamino-azetidin-1-yl)-3-fluoro-phenyl]-[5,5-dimethyl-7-(quinoline-8-sulfonyl)-6,7-dihydro-5H-pyrrolo[2,3-d]pyrimidin-2-yl]-amine;
(S)—N-(3-methoxy-4-(3-methylpiperazin-1-yl)phenyl)-5,5-dimethyl-7-(quinolin-8-ylsulfonyl)-6,7-dihydro-5H-pyrrolo[2,3-d]pyrimidin-2-amine;
N-(2-((2-((4-(4-(dimethylamino)piperidin-1-yl)-2-methoxyphenyl)amino)-7H-pyrrolo[2,3-d]pyrimidin-7-yl)methyl)phenyl)-N-methylmethanesulfonamide;
(S)—N-(3-((2-((2-methoxy-4-(3-methylpiperazin-1-yl)phenyl)amino)-7H-pyrrolo[2,3-d]pyrimidin-7-yl)methyl)pyridin-2-yl)-N-methylmethanesulfonamide;
N-(4-(4-aminopiperidin-1-yl)phenyl)-5,5-dimethyl-7-(quinolin-8-ylsulfonyl)-6,7-dihydro-5H-pyrrolo[2,3-d]pyrimidin-2-amine;
(S)-2-((6-((4-(4-(2-hydroxypropyl)piperazin-1-yl)phenyl)amino)-1H-pyrrolo[3,2-c]pyridin-1-yl)sulfonyl)benzonitrile;
(S)—N-(2-((2-((4-(4-(2-hydroxypropyl)piperazin-1-yl)phenyl)amino)-7H-pyrrolo[2,3-d]pyrimidin-7-yl)methyl)phenyl)-N-methylmethanesulfonamide;
N-(3-((2-((1-(2-(dimethylamino)ethyl)-2-oxoindolin-5-yl)amino)-7H-pyrrolo[2,3-d]pyrimidin-7-yl)methyl)pyridin-2-yl)-N-methylmethanesulfonamide;
N-(3-((2-((4-(4-(dimethylamino)piperidin-1-yl)-2-methoxyphenyl)amino)-7H-pyrrolo[2,3-d]pyrimidin-7-yl)methyl)pyridin-2-yl)-N-methylmethanesulfonamide;
N-(2-methoxy-4-(piperazin-1-yl)phenyl)-3,3-dimethyl-1-(quinolin-8-ylsulfonyl)-2,3-dihydro-1H-pyrrolo[3,2-c]pyridin-6-amine;
N-(3-((2-((4-(4-((2-hydroxyethyl)(methyl)amino)piperidin-1-yl)phenyl)amino)-7H-pyrrolo[2,3-d]pyrimidin-7-yl)methyl)pyridin-2-yl)-N-methylmethanesulfonamide;
(S)—N-cyclopropyl-N-(3-((2-((4-(3-methylpiperazin-1-yl)phenyl)amino)-7H-pyrrolo[2,3-d]pyrimidin-7-yl)methyl)pyridin-2-yl)methanesulfonamide;
N-(2-((2-((4-(4-hydroxy-4-methylpiperidin-1-yl)phenyl)amino)-7H-pyrrolo[2,3-d]pyrimidin-7-yl)methyl)phenyl)-N-methylmethanesulfonamide;
1-(4-((5,5-dimethyl-7-(quinolin-8-ylsulfonyl)-6,7-dihydro-5H-pyrrolo[2,3-d]pyrimidin-2-yl)amino)phenyl)-4-methylpiperidin-4-ol;
N-(4-(4-(dimethylamino)piperidin-1-yl)-3-fluorophenyl)-5,5-dimethyl-7-(quinolin-8-ylsulfonyl)-6,7-dihydro-5H-pyrrolo[2,3-d]pyrimidin-2-amine;
N-cyclopropyl-N-(3-((2-((4-(piperazin-1-yl)phenyl)amino)-7H-pyrrolo[2,3-d]pyrimidin-7-yl)methyl)pyridin-2-yl)methanesulfonamide;
(S)-1-(4-(4-((5,5-dimethyl-7-(quinolin-8-ylsulfonyl)-6,7-dihydro-5H-pyrrolo[2,3-d]pyrimidin-2-yl)amino)phenyl)piperazin-1-yl)propan-2-ol;
(S)—N-(2-methoxy-6-((2-((4-(3-methylpiperazin-1-yl)phenyl)amino)-7H-pyrrolo[2,3-d]pyrimidin-7-yl)methyl)phenyl)-N-methylmethanesulfonamide;
N-methyl-N-(3-((2-((4-(4-(piperazin-1-yl)piperidin-1-yl)phenyl)amino)-7H-pyrrolo[2,3-d]pyrimidin-7-yl)methyl)pyridin-2-yl)methanesulfonamide;
N-(3-((2-((4-(3-ethylpiperazin-1-yl)phenyl)amino)-7H-pyrrolo[2,3-d]pyrimidin-7-yl)methyl)pyridin-2-yl)-N-methylmethanesulfonamide;
N-methyl-N-(3-((2-((4-(4-morpholinopiperidin-1-yl)phenyl)amino)-7H-pyrrolo[2,3-d]pyrimidin-7-yl)methyl)pyridin-2-yl)methanesulfonamide;
N-methyl-N-(2-((2-((4-(4-(methylamino)piperidin-1-yl)phenyl)amino)-7H-pyrrolo[2,3-d]pyrimidin-7-yl)methyl)phenyl)methanesulfonamide;
N-(4-(4-(dimethylamino)piperidin-1-yl)-3-methylphenyl)-5,5-dimethyl-7-(quinolin-8-ylsulfonyl)-6,7-dihydro-5H-pyrrolo[2,3-d]pyrimidin-2-amine;
N-(2-((2-((4-(4-(dimethylamino)piperidin-1-yl)-3-methylphenyl)amino)-7H-pyrrolo[2,3-d]pyrimidin-7-yl)methyl)phenyl)-N-methylmethanesulfonamide;
N-(4-(4-(dimethylamino)piperidin-1-yl)-2-methoxyphenyl)-5,5-dimethyl-7-(quinolin-8-ylsulfonyl)-6,7-dihydro-5H-pyrrolo[2,3-d]pyrimidin-2-amine;
N-(3-((2-(indolin-5-ylamino)-7H-pyrrolo[2,3-d]pyrimidin-7-yl)methyl)pyridin-2-yl)-N-methylmethanesulfonamide;
N-(2-methoxy-6-((2-((4-(piperazin-1-yl)phenyl)amino)-7H-pyrrolo[2,3-d]pyrimidin-7-yl)methyl)phenyl)-N-methylmethanesulfonamide;
N-(2-((2-((4-(3-ethylpiperazin-1-yl)phenyl)amino)-7H-pyrrolo[2,3-d]pyrimidin-7-yl)methyl)phenyl)-N-methylmethanesulfonamide;
N-methyl-N-(3-((2-((4-(piperidin-4-yloxy)phenyl)amino)-7H-pyrrolo[2,3-d]pyrimidin-7-yl)methyl)pyridin-2-yl)methanesulfonamide;
N-(2-((2-((4-(4-cyclopropylpiperazin-1-yl)phenyl)amino)-7H-pyrrolo[2,3-d]pyrimidin-7-yl)methyl)phenyl)-N-methylmethanesulfonamide;
N-(4-(3-ethylpiperazin-1-yl)phenyl)-5,5-dimethyl-7-(quinolin-8-ylsulfonyl)-6,7-dihydro-5H-pyrrolo[2,3-d]pyrimidin-2-amine;
N-(2-((2-((4-(3-(aminomethyl)piperidin-1-yl)phenyl)amino)-7H-pyrrolo[2,3-d]pyrimidin-7-yl)methyl)phenyl)-N-methylmethanesulfonamide;
N-(4-(3-(aminomethyl)piperidin-1-yl)phenyl)-5,5-dimethyl-7-(quinolin-8-ylsulfonyl)-6,7-dihydro-5H-pyrrolo[2,3-d]pyrimidin-2-amine;
N-(3-((2-((4-(4-Cyclopropylpiperazin-1-yl)phenyl)amino)-7H-pyrrolo[2,3-d]pyrimidin-7-yl)methyl)pyridin-2-yl)-N-methylmethanesulfonamide;
N-methyl-N-(3-((2-((2-(1,2,3,6-tetrahydropyridin-4-yl)-1H-indol-5-yl)amino)-7H-pyrrolo[2,3-d]pyrimidin-7-yl)methyl)pyridin-2-yl)methanesulfonamide;
N-(4-methoxy-2-((2-((4-(piperazin-1-yl)phenyl)amino)-7H-pyrrolo[2,3-d]pyrimidin-7-yl)methyl)phenyl)-N-methylmethanesulfonamide;
2-(4-(4-((7-(2-(N-methylmethylsulfonamido)benzyl)-7H-pyrrolo[2,3-d]pyrimidin-2-yl)amino)phenyl)piperazin-1-yl)acetic acid;
N-(3-((2-((4-((S)-4-((S)-2-hydroxypropyl)-3-methylpiperazin-1-yl)phenyl)amino)-7H-pyrrolo[2,3-d]pyrimidin-7-yl)methyl)pyridin-2-yl)-N-methylmethanesulfonamide;
(R)-1-(4-(4-((5,5-dimethyl-7-(quinolin-8-ylsulfonyl)-6,7-dihydro-5H-pyrrolo[2,3-d]pyrimidin-2-yl)amino)phenyl)piperazin-1-yl)propan-2-ol;
(R)—N-(2-((2-((4-(4-(2-hydroxypropyl)piperazin-1-yl)phenyl)amino)-7H-pyrrolo[2,3-d]pyrimidin-7-yl)methyl)phenyl)-N-methylmethanesulfonamide;
(R)—N-(3-((2-((4-(4-(2-hydroxypropyl)piperazin-1-yl)phenyl)amino)-7H-pyrrolo[2,3-d]pyrimidin-7-yl)methyl)pyridin-2-yl)-N-methylmethanesulfonamide;
N-(3((2-((1H-indol-5-yl)amino)-7H-pyrrolo[2,3-d]pyrimidin-7-yl)methyl)pyridin-2-yl)-N-methylmethanesulfonamide;
N-methyl-N-(3-((2-((4-(5-oxomorpholin-2-yl)phenyl)amino)-7H-pyrrolo[2,3-d]pyrimidin-7-yl)methyl)pyridin-2-yl)methanesulfonamide;
N-methyl-N-(3-((2-((4-(2-oxo-1,2-dihydropyridin-4-yl)phenyl)amino)-7H-pyrrolo[2,3-d]pyrimidin-7-yl)methyl)pyridin-2-yl)methanesulfonamide;
(R)—N-(2-methoxy-6-((2-((4-(3-methylpiperazin-1-yl)phenyl)amino)-7H-pyrrolo[2,3-d]pyrimidin-7-yl)methyl)phenyl)-N-methylmethanesulfonamide;
(R)—N-(5-methoxy-2-((2-((4-(3-methylpiperazin-1-yl)phenyl)amino)-7H-pyrrolo[2,3-d]pyrimidin-7-yl)methyl)phenyl)-N-methylmethanesulfonamide;
(R)—N-(4-methoxy-2-((2-((4-(3-methylpiperazin-1-yl)phenyl)amino)-7H-pyrrolo[2,3-d]pyrimidin-7-yl)methyl)phenyl)-N-methylmethanesulfonamide;
N1-(5,5-dimethyl-7-(quinolin-8-ylsulfonyl)-6,7-dihydro-5H-pyrrolo[2,3-d]pyrimidin-2-yl)-N4-(piperidin-4-yl)benzene-1,4-diamine;
N-methyl-N-(2-((2-((4-(piperidin-4-ylamino)phenyl)amino)-7H-pyrrolo[2,3-d]pyrimidin-7-yl)methyl)phenyl)methanesulfonamide;
(S)—N-(4-methoxy-2-((2-((4-(3-methylpiperazin-1-yl)phenyl)amino)-7H-pyrrolo[2,3-d]pyrimidin-7-yl)methyl)phenyl)-N-methylmethanesulfonamide;
N-methyl-N-(2-((2-((4-(5-oxomorpholin-2-yl)phenyl)amino)-7H-pyrrolo[2,3-d]pyrimidin-7-yl)methyl)phenyl)methanesulfonamide;
N-methyl-N-(3-((2-((2-(piperidin-4-yl)-1H-indol-5-yl)amino)-7H-pyrrolo[2,3-d]pyrimidin-7-yl)methyl)pyridin-2-yl)methanesulfonamide;
(S)—N-methyl-N-(2-((2-((4-(3-methylpiperazin-1-yl)phenyl)amino)-7H-pyrrolo[2,3-d]pyrimidin-7-yl)methyl)pyridin-3-yl)methanesulfonamide;
N-(3-((2-((4-((S)-4-((R)-2-hydroxypropyl)-3-methylpiperazin-1-yl)phenyl)amino)-7H-pyrrolo[2,3-d]pyrimidin-7-yl)methyl)pyridin-2-yl)-N-methylmethanesulfonamide;
(R)-2-((6-((4-(4-(2-hydroxypropyl)piperazin-1-yl)phenyl)amino)-1H-pyrrolo[3,2-c]pyridin-1-yl)sulfonyl)benzonitrile;
N-(2-((2-((4-(4-(dimethylamino)piperidin-1-yl)-3-methoxyphenyl)amino)-7H-pyrrolo[2,3-d]pyrimidin-7-yl)methyl)phenyl)-N-methylmethanesulfonamide;
(S)—N-methyl-N-(3-((2-((4-(3-methylpiperazine-1-carbonyl)phenyl)amino)-7H-pyrrolo[2,3-d]pyrimidin-7-yl)methyl)pyridin-2-yl)methanesulfonamide;
(S)—N-methyl-N-(3-((2-((4-((3-methylpiperazin-1-yl)methyl)phenyl)amino)-7H-pyrrolo[2,3-d]pyrimidin-7-yl)methyl)pyridin-2-yl)methanesulfonamide;
N-methyl-N-(3-((2-((4-(piperidin-4-ylamino)phenyl)amino)-7H-pyrrolo[2,3-d]pyrimidin-7-yl)methyl)pyridin-2-yl)methanesulfonamide;
(S)—N-(5-cyano-2-((2-((4-(3-methylpiperazin-1-yl)phenyl)amino)-7H-pyrrolo[2,3-d]pyrimidin-7-yl)methyl)phenyl)-N-methylmethanesulfonamide;
N-(4-(3-aminopiperidin-1-yl)phenyl)-5,5-dimethyl-7-(quinolin-8-ylsulfonyl)-6,7-dihydro-5H-pyrrolo[2,3-d]pyrimidin-2-amine;
(1R,4R)-4-((4-((5,5-dimethyl-7-(quinolin-8-ylsulfonyl)-6,7-dihydro-5H-pyrrolo[2,3-d]pyrimidin-2-yl)amino)phenyl)(methyl)amino)cyclohexanol;
N-methyl-N-(3-((2-((4-(6-oxo-1,6-dihydropyridin-3-yl)phenyl)amino)-7H-pyrrolo[2,3-d]pyrimidin-7-yl)methyl)pyridin-2-yl)methanesulfonamide;
N-(5-cyano-2-((2-((4-(4-(dimethylamino)piperidin-1-yl)phenyl)amino)-7H-pyrrolo[2,3-d]pyrimidin-7-yl)methyl)phenyl)-N-methylmethanesulfonamide;
N-methyl-N-(3-((2-((4-(morpholin-2-yl)phenyl)amino)-7H-pyrrolo[2,3-d]pyrimidin-7-yl)methyl)pyridin-2-yl)methanesulfonamide;
N-(4-methoxy-2-((2-((2-oxoindolin-5-yl)amino)-7H-pyrrolo[2,3-d]pyrimidin-7-yl)methyl)phenyl)-N-methylmethanesulfonamide;
N-(2-((2-((4-((R)-4-((S)-2-hydroxypropyl)-3-methylpiperazin-1-yl)phenyl)amino)-7H-pyrrolo[2,3-d]pyrimidin-7-yl)methyl)phenyl)-N-methylmethanesulfonamide;
N-(2-((2-((4-(4-(cyclopropylamino)piperidin-1-yl)phenyl)amino)-7H-pyrrolo[2,3-d]pyrimidin-7-yl)methyl)phenyl)-N-methylmethanesulfonamide;
N-(3-((2-((4-((R)-4-((S)-2-hydroxypropyl)-3-methylpiperazin-1-yl)phenyl)amino)-7H-pyrrolo[2,3-d]pyrimidin-7-yl)methyl)pyridin-2-yl)-N-methylmethanesulfonamide;
N-(2-((2-((4-((S)-4-((S)-2-hydroxypropyl)-3-methylpiperazin-1-yl)phenyl)amino)-7H-pyrrolo[2,3-d]pyrimidin-7-yl)methyl)phenyl)-N-methylmethanesulfonamide;
N-methyl-N-(3-((2-((2-(piperazine-1-carbonyl)-1H-indol-5-yl)amino)-7H-pyrrolo[2,3-d]pyrimidin-7-yl)methyl)pyridin-2-yl)methanesulfonamide;
(S)—N-(4-methoxy-2-((2-((4-(2-methylpiperazin-1-yl)phenyl)amino)-7H-pyrrolo[2,3-d]pyrimidin-7-yl)methyl)phenyl)-N-methylmethanesulfonamide;
N-methyl-N-(2-((2-((2-(piperazine-1-carbonyl)-1H-indol-5-yl)amino)-7H-pyrrolo[2,3-d]pyrimidin-7-yl)methyl)phenyl)methanesulfonamide;
2-(4-(4-((5,5-dimethyl-7-(quinolin-8-ylsulfonyl)-6,7-dihydro-5H-pyrrolo[2,3-d]pyrimidin-2-yl)amino)phenyl)piperazin-1-yl)acetic acid;
N-(2-((2-((3-fluoro-4-(piperazin-1-yl)phenyl)amino)-7H-pyrrolo[2,3-d]pyrimidin-7-yl)methyl)-4-methoxyphenyl)-N-methylmethanesulfonamide;
(S)—N-(3-methoxy-2-((2-((4-(3-methylpiperazin-1-yl)phenyl)amino)-7H-pyrrolo[2,3-d]pyrimidin-7-yl)methyl)phenyl)-N-methylmethanesulfonamide;
4-((5,5-dimethyl-7-(quinolin-8-ylsulfonyl)-6,7-dihydro-5H-pyrrolo[2,3-d]pyrimidin-2-yl)amino)-N-(1-methylpiperidin-4-yl)benzamide;
N-(2-((2-((4-(4-(dimethylamino)piperidin-1-yl)-3-methoxyphenyl)amino)-7H-pyrrolo[2,3-d]pyrimidin-7-yl)methyl)-4-methoxyphenyl)-N-methylmethanesulfonamide;
N-(2-((2-((4-(3-ethylpiperazin-1-yl)phenyl)amino)-7H-pyrrolo[2,3-d]pyrimidin-7-yl)methyl)-4-methoxyphenyl)-N-methylmethanesulfonamide;
N-(2-((2-((4-(4-(dimethylamino)piperidin-1-yl)-3-methylphenyl)amino)-7H-pyrrolo[2,3-d]pyrimidin-7-yl)methyl)-4-methoxyphenyl)-N-methylmethanesulfonamide;
N-(2-((2-((4-(4-(dimethylamino)piperidin-1-yl)phenyl)amino)-7H-pyrrolo[2,3-d]pyrimidin-7-yl)methyl)-4-methoxyphenyl)-N-methylmethanesulfonamide;
5,5-dimethyl-N-(4-(4-(methylamino)piperidin-1-yl)phenyl)-7-(naphthalen-1-ylsulfonyl)-6,7-dihydro-5H-pyrrolo[2,3-d]pyrimidin-2-amine;
N-(2-((2-((4-(3-aminopiperidin-1-yl)phenyl)amino)-7H-pyrrolo[2,3-d]pyrimidin-7-yl)methyl)phenyl)-N-methylmethanesulfonamide;
N-(4-methoxy-2-((2-((4-(4-(methylamino)piperidin-1-yl)phenyl)amino)-7H-pyrrolo[2,3-d]pyrimidin-7-yl)methyl)phenyl)-N-methylmethanesulfonamide;
(R)—N-(2-((2-((3-cyclopropyl-4-(3-methylpiperazin-1-yl)phenyl)amino)-7H-pyrrolo[2,3-d]pyrimidin-7-yl)methyl)phenyl)-N-methylmethanesulfonamide;
(S)—N-(3-methoxy-2-((2-((4-(3-methylpiperazin-1-yl)phenyl)amino)-7H-pyrrolo[2,3-d]pyrimidin-7-yl)methyl)phenyl)-N-methylmethanesulfonamide;
N-(4-(4-(dimethylamino)piperidin-1-yl)phenyl)-5-methyl-7-(quinolin-8-ylsulfonyl)-7H-pyrrolo[2,3-d]pyrimidin-2-amine;
5,5-dimethyl-7-(pyridin-2-yl)-N-(3-(1,2,3,6-tetrahydropyridin-4-yl)-1H-indol-5-yl)-6,7-dihydro-5H-pyrrolo[2,3-d]pyrimidin-2-amine;
(S)—N-(5-methoxy-2-((2-((4-(3-methylpiperazin-1-yl)phenyl)amino)-7H-pyrrolo[2,3-d]pyrimidin-7-yl)methyl)phenyl)-N-methylmethanesulfonamide;
N-(4-cyano-2-((2-((4-(piperazin-1-yl)phenyl)amino)-7H-pyrrolo[2,3-d]pyrimidin-7-yl)methyl)phenyl)-N-methylmethanesulfonamide;
N-methyl-N-(2-((5-methyl-2-((4-(piperazin-1-yl)phenyl)amino)-7H-pyrrolo[2,3-d]pyrimidin-7-yl)methyl)phenyl)methanesulfonamide;
N-(4-(difluoromethoxy)-2-((2-((4-(piperazin-1-yl)phenyl)amino)-7H-pyrrolo[2,3-d]pyrimidin-7-yl)methyl)phenyl)-N-methylmethanesulfonamide;
(R)—N-methyl-N-(2-((5-methyl-2-((4-(3-methylpiperazin-1-yl)phenyl)amino)-7H-pyrrolo[2,3-d]pyrimidin-7-yl)methyl)phenyl)methanesulfonamide;
N-methyl-N-(3-((5-methyl-2-((4-(piperazin-1-yl)phenyl)amino)-7H-pyrrolo[2,3-d]pyrimidin-7-yl)methyl)pyridin-2-yl)methanesulfonamide;
(S)—N-methyl-N-(3-((5-methyl-2-((4-(3-methylpiperazin-1-yl)phenyl)amino)-7H-pyrrolo[2,3-d]pyrimidin-7-yl)methyl)pyridin-2-yl)methanesulfonamide;
N-(4-(4-(dimethylamino)piperidin-1-yl)phenyl)-5,5-dimethyl-7-(pyridin-2-yl)-6,7-dihydro-5H-pyrrolo[2,3-d]pyrimidin-2-amine;
N-(3-((5-fluoro-2-((4-(piperazin-1-yl)phenyl)amino)-7H-pyrrolo[2,3-d]pyrimidin-7-yl)methyl)pyridin-2-yl)-N-methylmethanesulfonamide;
(S)-3-fluoro-N-(2-((2-((4-(3-methylpiperazin-1-yl)phenyl)amino)-7H-pyrrolo[2,3-d]pyrimidin-7-yl)methyl)phenyl)propane-1-sulfonamide;
7-(2-(difluoromethoxy)benzyl)-N-(4-(piperazin-1-yl)phenyl)-7H-pyrrolo[2,3-d]pyrimidin-2-amine;
(R)-7-(2-(difluoromethoxy)benzyl)-N-(4-(3-methylpiperazin-1-yl)phenyl)-7H-pyrrolo[2,3-d]pyrimidin-2-amine;
(R)-5,5-dimethyl-N-(4-(3-methylpiperazin-1-yl)phenyl)-7-(pyridin-2-yl)-6,7-dihydro-5H-pyrrolo[2,3-d]pyrimidin-2-amine;
N-(3-((2-((4-(4-(dimethylamino)piperidin-1-yl)phenyl)amino)-5-fluoro-7H-pyrrolo[2,3-d]pyrimidin-7-yl)methyl)pyridin-2-yl)-N-methylmethanesulfonamide;
N-(3-((5-cyano-2-((4-(4-(dimethylamino)piperidin-1-yl)phenyl)amino)-7H-pyrrolo[2,3-d]pyrimidin-7-yl)methyl)pyridin-2-yl)-N-methylmethanesulfonamide;
(S)-3-fluoro-N-(2-((2-((2-fluoro-4-(3-methylpiperazin-1-yl)phenyl)amino)-7H-pyrrolo[2,3-d]pyrimidin-7-yl)methyl)phenyl)propane-1-sulfonamide;
(S)-3-fluoro-N-(2-((2-((2-fluoro-4-(3-methylpiperazin-1-yl)phenyl)amino)-7H-pyrrolo[2,3-d]pyrimidin-7-yl)methyl)phenyl)-N-methylpropane-1-sulfonamide;
N-(2-((5-cyano-2-((4-(piperazin-1-yl)phenyl)amino)-7H-pyrrolo[2,3-d]pyrimidin-7-yl)methyl)phenyl)-N-methylmethanesulfonamide;
N-(3-((5-cyano-2-((4-(piperazin-1-yl)phenyl)amino)-7H-pyrrolo[2,3-d]pyrimidin-7-yl)methyl)pyridin-2-yl)-N-methylmethanesulfonamide;
(S)-3-fluoro-N-methyl-N-(2-((2-((4-(3-methylpiperazin-1-yl)phenyl)amino)-7H-pyrrolo[2,3-d]pyrimidin-7-yl)methyl)phenyl)propane-1-sulfonamide;
7-((2-(N-methylmethylsulfonamido)pyridin-3-yl)methyl)-2-((4-(piperazin-1-yl)phenyl)amino)-7H-pyrrolo[2,3-d]pyrimidine-5-carboxamide;
N-(2-(2-((4-(4-(dimethylamino)piperidin-1-yl)phenyl)amino)-7H-pyrrolo[2,3-d]pyrimidin-7-yl)phenyl)-N-methylmethanesulfonamide;
N-methyl-N-(2-((2-((4-(4-(piperazin-1-yl)piperidin-1-yl)phenyl)amino)-7H-pyrrolo[2,3-d]pyrimidin-7-yl)methyl)phenyl)methanesulfonamide;
N-(3-((2-((4-(3-(Fluoromethyl)piperazin-1-yl)phenyl)amino)-7H-pyrrolo[2,3-d]pyrimidin-7-yl)methyl)pyridin-2-yl)-N-methylmethanesulfonamide;
2-((1-(4-((5,5-Dimethyl-7-(quinolin-8-ylsulfonyl)-6,7-dihydro-5H-pyrrolo[2,3-d]pyrimidin-2-yl)amino)phenyl)piperidin-4-yl)(methyl)amino)ethan-1-ol;
N-methyl-N-(2-((2-((4-(piperazin-1-yl)phenyl)amino)-7H-pyrrolo[2,3-d]pyrimidin-7-yl)methyl)cyclohex-1-en-1-yl)methanesulfonamide;
5-((5,5-dimethyl-7-(pyridin-2-yl)-6,7-dihydro-5H-pyrrolo[2,3-d]pyrimidin-2-yl)amino)indolin-2-one;
2-((2-((4-(4-(dimethylamino)piperidin-1-yl)phenyl)amino)-7H-pyrrolo[2,3-d]pyrimidin-7-yl)methyl)-N,N-dimethylbenzamide;
5,5-dimethyl-7-(pyridin-3-yl)-N-(3-(1,2,3,6-tetrahydropyridin-4-yl)-1H-indol-5-yl)-6,7-dihydro-5H-pyrrolo[2,3-d]pyrimidin-2-amine;
(S)—N-(4-fluoro-2-((2-((4-(3-methylpiperazin-1-yl)phenyl)amino)-7H-pyrrolo[2,3-d]pyrimidin-7-yl)methyl)phenyl)-N-methylmethanesulfonamide;
N-(6-(2-((4-(4-(dimethylamino)piperidin-1-yl)phenyl)amino)-7H-pyrrolo[2,3-d]pyrimidin-7-yl)pyridin-2-yl)-N-methylmethanesulfonamide;
N-(1H-indol-5-yl)-5,5-dimethyl-7-(pyridin-2-yl)-6,7-dihydro-5H-pyrrolo[2,3-d]pyrimidin-2-amine;
(S)—N-methyl-N-(3-((6-((4-(3-methylpiperazin-1-yl)phenyl)amino)-1H-pyrazolo[3,4-d]pyrimidin-1-yl)methyl)pyridin-2-yl)methanesulfonamide;
5-((5,5-dimethyl-7-(pyridin-2-yl)-6,7-dihydro-5H-pyrrolo[2,3-d]pyrimidin-2-yl)amino)indolin-2-one;
N-(4-(4-(dimethylamino)piperidin-1-yl)-3-methoxyphenyl)-5, 5-dimethyl-7-(quinolin-8-ylsulfonyl)-6,7-dihydro-5H-pyrrolo[2,3-d]pyrimidin-2-amine;
1-(4-((5,5-dimethyl-7-(quinolin-8-ylsulfonyl)-6,7-dihydro-5H-pyrrolo[2,3-d]pyrimidin-2-yl)amino)phenyl)piperidin-3-ol;
N-(2-((2-((4-(3-hydroxypiperidin-1-yl)phenyl)amino)-7H-pyrrolo[2,3-d]pyrimidin-7-yl)methyl)phenyl)-N-methylmethanesulfonamide;
N-(2-((2-((4-(((1R,4R)-4-hydroxycyclohexyl)(methyl)amino)phenyl)amino)-7H-pyrrolo[2,3-d]pyrimidin-7-yl)methyl)phenyl)-N-methylmethanesulfonamide;
5, 5-dimethyl-N-(4-(3-(methylamino)piperidin-1-yl)phenyl)-7-(quinolin-8-ylsulfonyl)-6,7-dihydro-5H-pyrrolo[2,3-d]pyrimidin-2-amine;
N-(3-((2-((4-(3-hydroxypiperidin-1-yl)phenyl)amino)-7H-pyrrolo[2,3-d]pyrimidin-7-yl)methyl)pyridin-2-yl)-N-methylmethanesulfonamide;
N-(3-((2-((4-(((1R,4R)-4-hydroxycyclohexyl)(methyl)amino)phenyl)amino)-7H-pyrrolo[2,3-d]pyrimidin-7-yl)methyl)pyridin-2-yl)-N-methylmethanesulfonamide;
N-(4-(4-(dimethylamino)piperidin-1-yl)phenyl)-5,5-dimethyl-7-(naphthalen-1-ylsulfonyl)-6,7-dihydro-5H-pyrrolo[2,3-d]pyrimidin-2-amine;
1-methyl-7-((2-((4-(piperazin-1-yl)phenyl)amino)-7H-pyrrolo[2,3-d]pyrimidin-7-yl)methyl)-1,3-dihydrobenzo[c]isothiazole 2,2-dioxide;
N-(2-((2-((4-(3-ethylpiperazin-1-yl)phenyl)amino)-7H-pyrrolo[2,3-d]pyrimidin-7-yl)methyl)phenyl)-N-methylmethanesulfonamide;
N-(2-((2-((4-(3-ethylpiperazin-1-yl)phenyl)amino)-7H-pyrrolo[2,3-d]pyrimidin-7-yl)methyl)phenyl)-N-methylmethanesulfonamide;
N-methyl-N-(4-methyl-2-((2-((4-(piperazin-1-yl)phenyl)amino)-7H-pyrrolo[2,3-d]pyrimidin-7-yl)methyl)phenyl)methanesulfonamide;
5,5-dimethyl-7-(naphthalen-1-ylsulfonyl)-N-(4-(piperazin-1-yl)phenyl)-6,7-dihydro-5H-pyrrolo[2,3-d]pyrimidin-2-amine;
N-(2-((2-((4-(4-(dimethylamino)piperidin-1-yl)phenyl)amino)-7H-pyrrolo[2,3-d]pyrimidin-7-yl)methyl)-4-methylphenyl)-N-methylmethanesulfonamide;
(R)-5,5-dimethyl-N-(4-(3-methylpiperazin-1-yl)phenyl)-7-(naphthalen-1-ylsulfonyl)-6,7-dihydro-5H-pyrrolo[2,3-d]pyrimidin-2-amine;
(R)—N-methyl-N-(4-methyl-2-((2-((4-(3-methylpiperazin-1-yl)phenyl)amino)-7H-pyrrolo[2,3-d]pyrimidin-7-yl)methyl)phenyl)methanesulfonamide;
(S)-1-methyl-7-((2-((4-(3-methylpiperazin-1-yl)phenyl)amino)-7H-pyrrolo[2,3-d]pyrimidin-7-yl)methyl)-1,3-dihydrobenzo[c]isothiazole 2,2-dioxide;
7-((2-((4-(4-(dimethylamino)piperidin-1-yl)phenyl)amino)-7H-pyrrolo[2,3-d]pyrimidin-7-yl)methyl)-1-methyl-1,3-dihydrobenzo[c]isothiazole 2,2-dioxide;
(S)—N-methyl-N-(4-methyl-2-((2-((4-(3-methylpiperazin-1-yl)phenyl)amino)-7H-pyrrolo[2,3-d]pyrimidin-7-yl)methyl)phenyl)methanesulfonamide;
N-methyl-N-(2-(1-(2-((4-((S)-3-methylpiperazin-1-yl)phenyl)amino)-7H-pyrrolo[2,3-d]pyrimidin-7-yl)ethyl)phenyl)methanesulfonamide;
N-methyl-N-(3-((2-((4-(piperidin-4-yl)phenyl)amino)-7H-pyrrolo[2,3-d]pyrimidin-7-yl)methyl)pyridin-2-yl)methanesulfonamide;
5-methyl-N-(4-(piperazin-1-yl)phenyl)-7-(quinolin-8-ylsulfonyl)-7H-pyrrolo[2,3-d]pyrimidin-2-amine;
N-methyl-N-(2-((2-((4-(piperidin-4-yloxy)phenyl)amino)-7H-pyrrolo[2,3-d]pyrimidin-7-yl)methyl)phenyl)methanesulfonamide;
N-(4-(4-Cyclopropylpiperazin-1-yl)phenyl)-5,5-dimethyl-7-(quinolin-8-ylsulfonyl)-6,7-dihydro-5H-pyrrolo[2,3-d]pyrimidin-2-amine;
N-(5-methoxy-2-((2-((4-(piperazin-1-yl)phenyl)amino)-7H-pyrrolo[2,3-d]pyrimidin-7-yl)methyl)phenyl)-N-methylmethanesulfonamide;
(S)—N-methyl-N-(2-((2-((4-(3-methylpiperazine-1-carbonyl)phenyl)amino)-7H-pyrrolo[2,3-d]pyrimidin-7-yl)methyl)phenyl)methanesulfonamide;
N-methyl-N-(2-((2-((4-(3-(methylamino)piperidin-1-yl)phenyl)amino)-7H-pyrrolo[2,3-d]pyrimidin-7-yl)methyl)phenyl)methanesulfonamide;
(S)—N-methyl-N-(2-((2-((4-((3-methylpiperazin-1-yl)methyl)phenyl)amino)-7H-pyrrolo[2,3-d]pyrimidin-7-yl)methyl)phenyl)methanesulfonamide;
N-(3-((2-((4-(3-aminopiperidin-1-yl)phenyl)amino)-7H-pyrrolo[2,3-d]pyrimidin-7-yl)methyl)pyridin-2-yl)-N-methylmethanesulfonamide;
4-((7-(2-(N-methylmethylsulfonamido)benzyl)-7H-pyrrolo[2,3-d]pyrimidin-2-yl)amino)-N-(1-methylpiperidin-4-yl)benzamide;
N-(2-((2-((4-(4-(dimethylamino)piperidin-1-yl)phenyl)amino)-7H-pyrrolo[2,3-d]pyrimidin-7-yl)methyl)-4-fluorophenyl)-N-methylmethanesulfonamide;
N-methyl-N-(2-(1-(2-((4-((R)-3-methylpiperazin-1-yl)phenyl)amino)-7H-pyrrolo[2,3-d]pyrimidin-7-yl)ethyl)phenyl)methanesulfonamide;
N-(2-((2-((4-(4-(dimethylamino)piperidin-1-yl)-3-methylphenyl)amino)-7H-pyrrolo[2,3-d]pyrimidin-7-yl)methyl)-4-methylphenyl)-N-methylmethanesulfonamide;
(S)—N-methyl-N-(3-((6-((4-(3-methylpiperazin-1-yl)phenyl)amino)-1H-pyrazolo[4,3-c]pyridin-1-yl)methyl)pyridin-2-yl)methanesulfonamide;
N-(4-fluoro-2-((2-((3-methyl-4-(piperidin-4-yl)phenyl)amino)-7H-pyrrolo[2,3-d]pyrimidin-7-yl)methyl)phenyl)-N-methylmethanesulfonamide;
N-methyl-N-(2-((2-((3-methyl-4-(piperidin-4-yl)phenyl)amino)-7H-pyrrolo[2,3-d]pyrimidin-7-yl)methyl)phenyl)methanesulfonamide;
N-(2-(2-((4-(4-(dimethylamino)piperidin-1-yl)phenyl)amino)-5,5-dimethyl-5,6-dihydro-7H-pyrrolo[2,3-d]pyrimidin-7-yl)phenyl)methanesulfonamide;
N-(3-((5-cyano-6-((4-(4-(dimethylamino)piperidin-1-yl)phenyl)amino)-1H-pyrrolo[2,3-b]pyridin-1-yl)methyl)pyridin-2-yl)-N-methylmethanesulfonamide;
N-methyl-N-(4-methyl-2-((2-((2-oxoindolin-5-yl)amino)-7H-pyrrolo[2,3-d]pyrimidin-7-yl)methyl)phenyl)methanesulfonamide;
(R)—N-methyl-N-(2-((2-((4-(3-methylpiperazin-1-yl)phenyl)amino)-7H-pyrrolo[2,3-d]pyrimidin-7-yl)methyl)phenyl)acetamide;
N-(2-((2-((4-(4-(dimethylamino)piperidin-1-yl)phenyl)amino)-7H-pyrrolo[2,3-d]pyrimidin-7-yl)methyl)phenyl)-N-methylacetamide;
N-methyl-N-(4-methyl-2-((2-((4-(piperidin-4-yl)phenyl)amino)-7H-pyrrolo[2,3-d]pyrimidin-7-yl)methyl)phenyl)methanesulfonamide;
N-(4-(4-(dimethylamino)piperidin-1-yl)phenyl)-7-(2-(1-(methylsulfonyl)ethyl)benzyl)-7H-pyrrolo[2,3-d]pyrimidin-2-amine;
N-(4-fluoro-2-((2-((2-oxoindolin-5-yl)amino)-7H-pyrrolo[2,3-d]pyrimidin-7-yl)methyl)phenyl)-N-methylmethanesulfonamide;
(R)—N-(4-fluoro-2-((2-((4-(3-methylpiperazin-1-yl)phenyl)amino)-7H-pyrrolo[2,3-d]pyrimidin-7-yl)methyl)phenyl)-N-methylmethanesulfonamide;
N-(2-((2-((4-(4-(dimethylamino)piperidin-1-yl)-3-methylphenyl)amino)-7H-pyrrolo[2,3-d]pyrimidin-7-yl)methyl)-4-fluorophenyl)-N-methylmethanesulfonamide;
(S)—N-(3-((2-((3-cyano-4-(3-methylpiperazin-1-yl)phenyl)amino)-7H-pyrrolo[2,3-d]pyrimidin-7-yl)methyl)pyridin-2-yl)-N-methylmethanesulfonamide;
N-(4-chloro-2-((2-((4-(4-(dimethylamino)piperidin-1-yl)phenyl)amino)-7H-pyrrolo[2,3-d]pyrimidin-7-yl)methyl)phenyl)-N-methylmethanesulfonamide;
N-methyl-N-(3-((2-((3-methyl-4-(piperidin-4-yl)phenyl)amino)-7H-pyrrolo[2,3-d]pyrimidin-7-yl)methyl)pyridin-2-yl)methanesulfonamide;
7-((2-(difluoromethoxy)pyridin-3-yl)methyl)-N-(4-(piperazin-1-yl)phenyl)-7H-pyrrolo[2,3-d]pyrimidin-2-amine;
N-(2-((2-((4-(4-aminopiperidin-1-yl)phenyl)amino)-7H-pyrrolo[2,3-d]pyrimidin-7-yl)methyl)-4-fluorophenyl)-N-methylmethanesulfonamide;
N-(2-((2-((4-(3-aminopiperidin-1-yl)phenyl)amino)-7H-pyrrolo[2,3-d]pyrimidin-7-yl)methyl)-4-fluorophenyl)-N-methylmethanesulfonamide;
N-(4-fluoro-2-((2-((3-methoxy-4-(piperazin-1-yl)phenyl)amino)-7H-pyrrolo[2,3-d]pyrimidin-7-yl)methyl)phenyl)-N-methylmethanesulfonamide;
N-(4-fluoro-2-((2-((3-fluoro-4-(piperazin-1-yl)phenyl)amino)-7H-pyrrolo[2,3-d]pyrimidin-7-yl)methyl)phenyl)-N-methylmethanesulfonamide;
(S)—N-(2-((2-((3-cyano-4-(3-methylpiperazin-1-yl)phenyl)amino)-7H-pyrrolo[2,3-d]pyrimidin-7-yl)methyl)-4-fluorophenyl)-N-methylmethanesulfonamide;
7-(2-(1-(methylsulfonyl)ethyl)benzyl)-N-(4-(piperazin-1-yl)phenyl)-7H-pyrrolo[2,3-d]pyrimidin-2-amine;
N-(4-fluoro-2-((2-((4-(piperazin-1-yl)phenyl)amino)-7H-pyrrolo[2,3-d]pyrimidin-7-yl)methyl)phenyl)-N-methylmethanesulfonamide;
N-(2-((2-((4-(4-(dimethylamino)cyclohexyl)phenyl)amino)-7H-pyrrolo[2,3-d]pyrimidin-7-yl)methyl)-4-fluorophenyl)-N-methylmethanesulfonamide;
N-methyl-N-(4-methyl-2-((2-((3-methyl-4-(piperidin-4-yl)phenyl)amino)-7H-pyrrolo[2,3-d]pyrimidin-7-yl)methyl)phenyl)methanesulfonamide;
N-(4-fluoro-2-((2-((4-(piperidin-4-yl)phenyl)amino)-7H-pyrrolo[2,3-d]pyrimidin-7-yl)methyl)phenyl)-N-methylmethanesulfonamide;
(S)—N-(2-((2-((3-fluoro-4-(3-methylpiperazin-1-yl)phenyl)amino)-7H-pyrrolo[2,3-d]pyrimidin-7-yl)methyl)-4-methylphenyl)-N-methylmethanesulfonamide;
(S)—N-methyl-N-(2-((2-((4-(3-methylpiperazin-1-yl)phenyl)amino)-7H-pyrrolo[2,3-d]pyrimidin-7-yl)methyl)-4-(trifluoromethyl)phenyl)methanesulfonamide;
N-(3-((2-((4-(1-(2-hydroxyethyl)piperidin-4-yl)-3-methylphenyl)amino)-7H-pyrrolo[2,3-d]pyrimidin-7-yl)methyl)pyridin-2-yl)-N-methylmethanesulfonamide;
N-(3-((2-((4-(1-(2-(dimethylamino)ethyl)piperidin-4-yl)-3-methylphenyl)amino)-7H-pyrrolo[2,3-d]pyrimidin-7-yl)methyl)pyridin-2-yl)-N-methylmethanesulfonamide;
N-methyl-N-(2-((2-((4-(piperazin-1-yl)phenyl)amino)-7H-pyrrolo[2,3-d]pyrimidin-7-yl)methyl)-4-(trifluoromethyl)phenyl)methanesulfonamide;
(S)—N-(4-chloro-2-((2-((4-(3-methylpiperazin-1-yl)phenyl)amino)-7H-pyrrolo[2,3-d]pyrimidin-7-yl)methyl)phenyl)-N-methylmethanesulfonamide;
N-(4-fluoro-2-((2-((4-(4-(2-hydroxyethyl)piperazin-1-yl)phenyl)amino)-7H-pyrrolo[2,3-d]pyrimidin-7-yl)methyl)phenyl)-N-methylmethanesulfonamide;
N-(2-((2-((3-fluoro-4-(piperazin-1-yl)phenyl)amino)-7H-pyrrolo[2,3-d]pyrimidin-7-yl)methyl)-4-methylphenyl)-N-methylmethanesulfonamide;
N-(2-((2-((3-methoxy-4-(piperazin-1-yl)phenyl)amino)-7H-pyrrolo[2,3-d]pyrimidin-7-yl)methyl)-4-methylphenyl)-N-methylmethanesulfonamide;
(S)—N-methyl-N-(3-((2-((4-(3-methylpiperazin-1-yl)phenyl)amino)-7H-pyrrolo[2,3-d]pyrimidin-7-yl)methyl)pyrazin-2-yl)methanesulfonamide;
N-methyl-4-((7-((3-(N-methylmethylsulfonamido)pyrazin-2-yl)methyl)-7H-pyrrolo[2,3-d]pyrimidin-2-yl)amino)benzamide;
N-(3-((2-((4-(4-(dimethylamino)piperidin-1-yl)phenyl)amino)-7H-pyrrolo[2,3-d]pyrimidin-7-yl)methyl)pyrazin-2-yl)-N-methylmethanesulfonamide;
N-(2-((2-((4-(4-aminopiperidin-1-yl)phenyl)amino)-7H-pyrrolo[2,3-d]pyrimidin-7-yl)methyl)-4-methylphenyl)-N-methylmethanesulfonamide;
N-(2-((2-((4-(3-aminopiperidin-1-yl)phenyl)amino)-7H-pyrrolo[2,3-d]pyrimidin-7-yl)methyl)-4-methylphenyl)-N-methylmethanesulfonamide;
N-(2-((2-((4-(4-(dimethylamino)cyclohexyl)phenyl)amino)-7H-pyrrolo[2,3-d]pyrimidin-7-yl)methyl)phenyl)-N-methylmethanesulfonamide;
N-(2-((2-((4-(4-(dimethylamino)cyclohexyl)phenyl)amino)-7H-pyrrolo[2,3-d]pyrimidin-7-yl)methyl)-4-methylphenyl)-N-methylmethanesulfonamide;
N-(3-((2-((4-(4-(dimethylamino)piperidin-1-yl)-3-methylphenyl)amino)-7H-pyrrolo[2,3-d]pyrimidin-7-yl)methyl)-5-methylpyridin-2-yl)-N-methylmethanesulfonamide;
N-methyl-4-((7-((2-(N-methylmethylsulfonamido)pyridin-3-yl)methyl)-7H-pyrrolo[2,3-d]pyrimidin-2-yl)amino)benzamide;
4-((7-(5-fluoro-2-(N-methylmethylsulfonamido)benzyl)-7H-pyrrolo[2,3-d]pyrimidin-2-yl)amino)-N-methylbenzamide;
4-((7-(5-methoxy-2-(N-methylmethylsulfonamido)benzyl)-7H-pyrrolo[2,3-d]pyrimidin-2-yl)amino)-N-methylbenzamide;
(S)—N-methyl-N-(3-((2-((4-(3-methylpiperazin-1-yl)phenyl)amino)-7H-pyrrolo[2,3-d]pyrimidin-7-yl)methyl)pyridin-2-yl)methanesulfonamide hydrochloride;
N-methyl-4-((7-(5-methyl-2-(N-methylmethylsulfonamido)benzyl)-7H-pyrrolo[2,3-d]pyrimidin-2-yl)amino)benzamide; and
N-methyl-4-((7-(2-(N-methylmethylsulfonamido)benzyl)-7H-pyrrolo[2,3-d]pyrimidin-2-yl)amino)benzamide.

56. The compound according to claim 1, which is selected from the group consisting of:
N-methyl-N-(5-methyl-3-((2-((4-(piperazin-1-yl)phenyl)amino)-7H-pyrrolo[2,3-d]pyrimidin-7-yl)methyl)pyridin-2-yl)methanesulfonamide;
(R)—N-methyl-N-(5-methyl-3-((2-((4-(3-methylpiperazin-1-yl)phenyl)amino)-7H-pyrrolo[2,3-d]pyrimidin-7-yl)methyl)pyridin-2-yl)methanesulfonamide;
(S)—N-methyl-N-(5-methyl-3-((2-((4-(3-methylpiperazin-1-yl)phenyl)amino)-7H-pyrrolo[2,3-d]pyrimidin-7-yl)methyl)pyridin-2-yl)methanesulfonamide;
N-(3-((2-((4-(4-(2-hydroxyethyl)piperazin-1-yl)phenyl)amino)-7H-pyrrolo[2,3-d]pyrimidin-7-yl)methyl)-5-methylpyridin-2-yl)-N-methylmethanesulfonamide;
N-(3-((2-((4-(4-(dimethylamino)piperidin-1-yl)phenyl)amino)-7H-pyrrolo[2,3-d]pyrimidin-7-yl)methyl)-5-methylpyridin-2-yl)-N-methylmethanesulfonamide;
7-(isoquinolin-1-ylsulfonyl)-N-(4-(piperazin-1-yl)phenyl)-7H-pyrrolo[2,3-d]pyrimidin-2-amine;
7-(isoquinolin-8-ylsulfonyl)-N-(4-(piperazin-1-yl)phenyl)-7H-pyrrolo[2,3-d]pyrimidin-2-amine;
7-(benzylsulfonyl)-N-(4-(piperazin-1-yl)phenyl)-7H-pyrrolo[2,3-d]pyrimidin-2-amine;
N-(4-(piperazin-1-yl)phenyl)-7-((pyridin-2-ylmethyl)sulfonyl)-7H-pyrrolo[2,3-d]pyrimidin-2-amine;
8-((2-((4-(piperazin-1-yl)phenyl)amino)-7H-pyrrolo[2,3-d]pyrimidin-7-yl)sulfonyl)-3,4-dihydroquinolin-2(1H)-one;
8-((2-((4-(piperazin-1-yl)phenyl)amino)-7H-pyrrolo[2,3-d]pyrimidin-7-yl)sulfonyl)-3,4-dihydro-1H-benzo[c][1,2]thiazine 2,2-dioxide;
7-((1H-indol-7-yl)sulfonyl)-N-(4-(piperazin-1-yl)phenyl)-7H-pyrrolo[2,3-d]pyrimidin-2-amine;
7-(benzo[d]thiazol-4-ylsulfonyl)-N-(4-(piperazin-1-yl)phenyl)-7H-pyrrolo[2,3-d]pyrimidin-2-amine;
7-(benzo[d]thiazol-4-ylsulfonyl)-5,5-dimethyl-N-(4-(piperazin-1-yl)phenyl)-6,7-dihydro-5H-pyrrolo[2,3-d]pyrimidin-2-amine;
7-((2-chlorophenyl)sulfonyl)-5,5-dimethyl-N-(4-(piperazin-1-yl)phenyl)-6,7-dihydro-5H-pyrrolo[2,3-d]pyrimidin-2-amine;
N-(2-((5,5-dimethyl-2-((4-(piperazin-1-yl)phenyl)amino)-5,6-dihydro-7H-pyrrolo[2,3-d]pyrimidin-7-yl)methyl)phenyl)-N-methylmethanesulfonamide;
5-(methoxymethyl)-N-(4-(piperazin-1-yl)phenyl)-7-(quinolin-8-ylsulfonyl)-6,7-dihydro-5H-pyrrolo[2,3-d]pyrimidin-2-amine;
5-methyl-N-(4-(piperazin-1-yl)phenyl)-7-(quinolin-8-ylsulfonyl)-6,7-dihydro-5H-pyrrolo[2,3-d]pyrimidin-2-amine;
N-(2-((2-((2-methoxy-4-(piperidin-4-yl)phenyl)amino)-7H-pyrrolo[2,3-d]pyrimidin-7-yl)methyl)phenyl)-N-methylmethanesulfonamide;
N-(2-((2-((2-methoxy-4-(1-methylpiperidin-4-yl)phenyl)amino)-7H-pyrrolo[2,3-d]pyrimidin-7-yl)methyl)phenyl)-N-methylmethanesulfonamide;
N-(2-((2-((2-fluoro-4-(piperidin-4-yl)phenyl)amino)-7H-pyrrolo[2,3-d]pyrimidin-7-yl)methyl)phenyl)-N-methylmethanesulfonamide;
(2-((2-((4-(piperazin-1-yl)phenyl)amino)-7H-pyrrolo[2,3-d]pyrimidin-7-yl)methyl)phenyl)methanesulfonamide;
N-methyl-N-(2-(2-(2-((4-(piperazin-1-yl)phenyl)amino)-7H-pyrrolo[2,3-d]pyrimidin-7-yl)ethyl)phenyl)methanesulfonamide;
2-((6-((4-(2,5-dimethylpiperazin-1-yl)phenyl)amino)-1H-pyrrolo[3,2-c]pyridin-1-yl)sulfonyl)benzonitrile;
N-methyl-N-(2-((2-((4-(piperazin-1-yl)phenyl)amino)-7H-pyrrolo[2,3-d]pyrimidin-7-yl)methyl)phenyl)ethanesulfonamide;
N-methyl-N-(2-((2-((4-(piperazin-1-yl)phenyl)amino)-7H-pyrrolo[2,3-d]pyrimidin-7-yl)methyl)phenyl)propane-2-sulfonamide;
(S)—N-methyl-N-(3-((2-((4-(3-methylpiperazin-1-yl)phenyl)amino)-7H-pyrrolo[2,3-d]pyrimidin-7-yl)methyl)pyridin-4-yl)methanesulfonamide;
(S)—N-methyl-N-(4-((2-((4-(3-methylpiperazin-1-yl)phenyl)amino)-7H-pyrrolo[2,3-d]pyrimidin-7-yl)methyl)pyridin-3-yl)methanesulfonamide;
N-(3-((2-((4-(piperazin-1-yl)phenyl)amino)-7H-pyrrolo[2,3-d]pyrimidin-7-yl)methyl)pyridin-2-yl)methanesulfonamide;
(R)—N-(3-((2-((4-(3-methylpiperazin-1-yl)phenyl)amino)-7H-pyrrolo[2,3-d]pyrimidin-7-yl)methyl)pyridin-2-yl)methanesulfonamide;
N-(3-((2-((2-oxoindolin-5-yl)amino)-7H-pyrrolo[2,3-d]pyrimidin-7-yl)methyl)pyridin-2-yl)methanesulfonamide;
N-(3-((2-((4-(piperazin-1-yl)phenyl)amino)-7H-pyrrolo[2,3-d]pyrimidin-7-yl)methyl)pyridin-2-yl)cyclopropanesulfonamide;
(S)—N-(3-((2-((4-(3-methylpiperazin-1-yl)phenyl)amino)-7H-pyrrolo[2,3-d]pyrimidin-7-yl)methyl)pyridin-2-yl)methanesulfonamide;
(S)—N-methyl-N-(2-((2-((4-(2-methylpiperazin-1-yl)phenyl)amino)-7H-pyrrolo[2,3-d]pyrimidin-7-yl)methyl)pyridin-3-yl)methanesulfonamide;
(S)—N-methyl-N-(3-((2-((4-(2-methylpiperazin-1-yl)phenyl)amino)-7H-pyrrolo[2,3-d]pyrimidin-7-yl)methyl)pyridin-4-yl)methanesulfonamide;
(S)—N-methyl-N-(4-((2-((4-(2-methylpiperazin-1-yl)phenyl)amino)-7H-pyrrolo[2,3-d]pyrimidin-7-yl)methyl)pyridin-3-yl)methanesulfonamide;
(S)—N-methyl-N-(3-((2-((4-(2-methylpiperazin-1-yl)phenyl)amino)-7H-pyrrolo[2,3-d]pyrimidin-7-yl)methyl)pyridin-2-yl)cyclopropanesulfonamide;
N-(3-((2-((4-(2,2-dimethylpiperazin-1-yl)phenyl)amino)-7H-pyrrolo[2,3-d]pyrimidin-7-yl)methyl)pyridin-2-yl)-N-methylmethanesulfonamide;
(R)—N-(3-((2-((4-(2-(hydroxymethyl)piperazin-1-yl)phenyl)amino)-7H-pyrrolo[2,3-d]pyrimidin-7-yl)methyl)pyridin-2-yl)-N-methylmethanesulfonamide;
(S)—N-(3-((2-((4-(2-ethylpiperazin-1-yl)phenyl)amino)-7H-pyrrolo[2,3-d]pyrimidin-7-yl)methyl)pyridin-2-yl)-N-methylmethanesulfonamide;
(S)—N-(3-((2-((3-fluoro-4-(2-methylpiperazin-1-yl)phenyl)amino)-7H-pyrrolo[2,3-d]pyrimidin-7-yl)methyl)pyridin-2-yl)-N-methylmethanesulfonamide;
N-methyl-N-(3-((2-((4-((2-(piperazin-1-yl)ethyl)amino)phenyl)amino)-7H-pyrrolo[2,3-d]pyrimidin-7-yl)methyl)pyridin-2-yl)methanesulfonamide;
N-(2-hydroxyethyl)-N-(2-((2-((2-oxoindolin-5-yl)amino)-7H-pyrrolo[2,3-d]pyrimidin-7-yl)methyl)phenyl)methanesulfonamide;
N-(2-hydroxyethyl)-N-(3-((2-((4-(piperazin-1-yl)phenyl)amino)-7H-pyrrolo[2,3-d]pyrimidin-7-yl)methyl)pyridin-2-yl)methanesulfonamide;
(R)—N-(2-hydroxyethyl)-N-(3-((2-((4-(3-methylpiperazin-1-yl)phenyl)amino)-7H-pyrrolo[2,3-d]pyrimidin-7-yl)methyl)pyridin-2-yl)methanesulfonamide;
(R)—N-(1-(hydroxymethyl)cyclopropyl)-N-(2-((2-((4-(3-methylpiperazin-1-yl)phenyl)amino)-7H-pyrrolo[2,3-d]pyrimidin-7-yl)methyl)phenyl)methanesulfonamide;
(R)—N-cyclopropyl-N-(3-((2-((4-(3-methylpiperazin-1-yl)phenyl)amino)-7H-pyrrolo[2,3-d]pyrimidin-7-yl)methyl)pyridin-2-yl)methanesulfonamide;
N-methyl-N-(2-((2-((4-(3-(pyrrolidin-1-yl)azetidin-1-yl)phenyl)amino)-7H-pyrrolo[2,3-d]pyrimidin-7-yl)methyl)phenyl)methanesulfonamide;
N-methyl-N-(2-((2-((4-(3-(piperazin-1-yl)azetidin-1-yl)phenyl)amino)-7H-pyrrolo[2,3-d]pyrimidin-7-yl)methyl)phenyl)methanesulfonamide;
N-(2-((2-((1-(2-hydroxyethyl)-2-oxo-1,2,3,4-tetrahydroquinolin-6-yl)amino)-7H-pyrrolo[2,3-d]pyrimidin-7-yl)methyl)phenyl)methanesulfonamide;
N-(2-((2-((1-(2-(dimethylamino)ethyl)-2-oxo-1,2,3,4-tetrahydroquinolin-6-yl)amino)-7H-pyrrolo[2,3-d]pyrimidin-7-yl)methyl)phenyl)methanesulfonamide;
N-(3-((2-((4-(4,7-diazaspiro[2.5]octan-7-yl)phenyl)amino)-7H-pyrrolo[2,3-d]pyrimidin-7-yl)methyl)pyridin-2-yl)-N-methylmethanesulfonamide;
N-methyl-N-(2-((2-((4-(morpholin-3-yl)phenyl)amino)-7H-pyrrolo[2,3-d]pyrimidin-7-yl)methyl)phenyl)methanesulfonamide;
N-(4-(4-(dimethylamino)cyclohexyl)phenyl)-5,5-dimethyl-7-(quinolin-8-ylsulfonyl)-6,7-dihydro-5H-pyrrolo[2,3-d]pyrimidin-2-amine;
N-methyl-N-(2-((2-((4-((S)-3-methylpiperazin-1-yl)phenyl)amino)-7H-pyrrolo[2,3-d]pyrimidin-7-yl)methyl)phenyl)methanesulfonimidamide;
N-methyl-N-(3-((2-((4-((S)-3-methylpiperazin-1-yl)phenyl)amino)-7H-pyrrolo[2,3-d]pyrimidin-7-yl)methyl)pyridin-2-yl)methanesulfonimidamide;
N-(2-cyano-6-((2-((4-(piperazin-1-yl)phenyl)amino)-7H-pyrrolo[2,3-d]pyrimidin-7-yl)methyl)phenyl)-N-methylmethanesulfonamide;
(R)—N-(2-cyano-6-((2-((4-(3-methylpiperazin-1-yl)phenyl)amino)-7H-pyrrolo[2,3-d]pyrimidin-7-yl)methyl)phenyl)-N-methylmethanesulfonamide;
(S)—N-(2-cyano-6-((2-((4-(3-methylpiperazin-1-yl)phenyl)amino)-7H-pyrrolo[2,3-d]pyrimidin-7-yl)methyl)phenyl)-N-methylmethanesulfonamide;
N,N-dimethyl-3-((2-((4-(piperazin-1-yl)phenyl)amino)-7H-pyrrolo[2,3-d]pyrimidin-7-yl)methyl)picolinamide;
(R)—N,N-dimethyl-3-((2-((4-(3-methylpiperazin-1-yl)phenyl)amino)-7H-pyrrolo[2,3-d]pyrimidin-7-yl)methyl)picolinamide;
N-(2-(5,5-dimethyl-2-((3-(1,2,3,6-tetrahydropyridin-4-yl)-1H-indol-5-yl)amino)-5,6-dihydro-7H-pyrrolo[2,3-d]pyrimidin-7-yl)phenyl)-N-methylmethanesulfonamide;
N-(2-(5,5-dimethyl-2-((3-(1,2,3,6-tetrahydropyridin-4-yl)-1H-indol-5-yl)amino)-5,6-dihydro-7H-pyrrolo[2,3-d]pyrimidin-7-yl)phenyl)-N-methylacetamide;
(S)—N-methyl-N-(2-(2-(2-((4-(3-methylpiperazin-1-yl)phenyl)amino)-7H-pyrrolo[2,3-d]pyrimidin-7-yl)propan-2-yl)phenyl)methanesulfonamide;
N-(2-(2-(2-((4-(4-(dimethylamino)piperidin-1-yl)phenyl)amino)-7H-pyrrolo[2,3-d]pyrimidin-7-yl)propan-2-yl)phenyl)-N-methylmethanesulfonamide;
(S)—N-methyl-N-(3-((2-((4-(3-methylpiperazin-1-yl)phenyl)amino)-7H-pyrrolo[2,3-d]pyrimidin-7-yl)methyl)pyridin-2-yl)acetamide;
(R)—N-(2-((5-cyano-6-((4-(3-methylpiperazin-1-yl)phenyl)amino)-1H-pyrrolo[2,3-b]pyridin-1-yl)methyl)phenyl)-N-methylmethanesulfonamide;
(S)—N-(2-((5-cyano-6-((4-(3-methylpiperazin-1-yl)phenyl)amino)-1H-pyrrolo[2,3-b]pyridin-1-yl)methyl)phenyl)-N-methylmethanesulfonamide;
(R)—N-methyl-N-(2-((5-((4-(3-methylpiperazin-1-yl)phenyl)amino)-3H-[1,2,3]triazolo[4,5-d]pyrimidin-3-yl)methyl)phenyl)methanesulfonamide;
(S)—N-(4-chloro-2-((2-((3-methyl-4-(3-methylpiperazin-1-yl)phenyl)amino)-7H-pyrrolo[2,3-d]pyrimidin-7-yl)methyl)phenyl)-N-methylmethanesulfonamide;
1,3-dimethyl-1-(3-((2-((4-(piperazin-1-yl)phenyl)amino)-7H-pyrrolo[2,3-d]pyrimidin-7-yl)methyl)pyridin-2-yl)urea;
(S)-1,3-dimethyl-1-(3-((2-((4-(3-methylpiperazin-1-yl)phenyl)amino)-7H-pyrrolo[2,3-d]pyrimidin-7-yl)methyl)pyridin-2-yl)urea;
(R)-1,3-dimethyl-1-(2-((2-((4-(3-methylpiperazin-1-yl)phenyl)amino)-7H-pyrrolo[2,3-d]pyrimidin-7-yl)methyl)phenyl)urea;
1-(2-((2-((4-(4-(dimethylamino)piperidin-1-yl)phenyl)amino)-7H-pyrrolo[2,3-d]pyrimidin-7-yl)methyl)phenyl)-1,3-dimethylurea;
N-(4-chloro-2-((2-((3-methyl-4-(piperidin-4-yl)phenyl)amino)-7H-pyrrolo[2,3-d]pyrimidin-7-yl)methyl)phenyl)-N-methylmethanesulfonamide;
(S)—N-methyl-N-(3-((2-((4-(2-methylpiperidin-4-yl)phenyl)amino)-7H-pyrrolo[2,3-d]pyrimidin-7-yl)methyl)pyridin-2-yl)methanesulfonamide;
N-(2-((2-((4-(1-(2-(dimethylamino)ethyl)piperidin-4-yl)-3-methylphenyl)amino)-7H-pyrrolo[2,3-d]pyrimidin-7-yl)methyl)phenyl)-N-methylmethanesulfonamide;
7-(2-(isopropylsulfonyl)phenyl)-5,5-dimethyl-N-(3-methyl-4-(piperidin-4-yl)phenyl)-6,7-dihydro-5H-pyrrolo[2,3-d]pyrimidin-2-amine;
2-(5,5-dimethyl-2-((3-methyl-4-(piperidin-4-yl)phenyl)amino)-5,6-dihydro-7H-pyrrolo[2,3-d]pyrimidin-7-yl)-N,N-dimethylbenzenesulfonamide;
(R)—N-methyl-N-(2-((2-((4-(3-methylpiperazin-1-yl)phenyl)amino)-7H-pyrrolo[2,3-d]pyrimidin-7-yl)methyl)-4-(trifluoromethyl)phenyl)methanesulfonamide;
N-methyl-N-(2-((2-((4-(piperidin-4-yl)phenyl)amino)-7H-pyrrolo[2,3-d]pyrimidin-7-yl)methyl)-4-(trifluoromethyl)phenyl)methanesulfonamide;
N-(2-((2-((4-(4-(2-hydroxyethyl)piperazin-1-yl)phenyl)amino)-7H-pyrrolo[2,3-d]pyrimidin-7-yl)methyl)-4-(trifluoromethyl)phenyl)-N-methylmethanesulfonamide;
N-(2-((2-((4-(4-(dimethylamino)piperidin-1-yl)phenyl)amino)-7H-pyrrolo[2,3-d]pyrimidin-7-yl)methyl)-4-(trifluoromethyl)phenyl)-N-methylmethanesulfonamide;
N-(4-chloro-2-((2-((4-(piperazin-1-yl)phenyl)amino)-7H-pyrrolo[2,3-d]pyrimidin-7-yl)methyl)phenyl)-N-methylmethanesulfonamide;
(R)—N-(4-chloro-2-((2-((4-(3-methylpiperazin-1-yl)phenyl)amino)-7H-pyrrolo[2,3-d]pyrimidin-7-yl)methyl)phenyl)-N-methylmethanesulfonamide;
N-(4-chloro-2-((2-((4-(4-(2-hydroxyethyl)piperazin-1-yl)phenyl)amino)-7H-pyrrolo[2,3-d]pyrimidin-7-yl)methyl)phenyl)-N-methylmethanesulfonamide;
N-(4-chloro-2-((2-((4-(4-(dimethylamino)cyclohexyl)phenyl)amino)-7H-pyrrolo[2,3-d]pyrimidin-7-yl)methyl)phenyl)-N-methylmethanesulfonamide;
(S)—N-(5-methoxy-3-((2-((4-(3-methylpiperazin-1-yl)phenyl)amino)-7H-pyrrolo[2,3-d]pyrimidin-7-yl)methyl)pyridin-2-yl)-N-methylmethanesulfonamide;
N-(4-((S)-3-methylpiperazin-1-yl)phenyl)-7-((2-(1-(methylsulfonyl)ethyl)pyridin-3-yl)methyl)-7H-pyrrolo[2,3-d]pyrimidin-2-amine;
N-(4-((R)-3-methylpiperazin-1-yl)phenyl)-7-(2-(1-(methylsulfonyl)ethyl)benzyl)-7H-pyrrolo[2,3-d]pyrimidin-2-amine;
(S)—N-(5-chloro-3-((2-((4-(3-methylpiperazin-1-yl)phenyl)amino)-7H-pyrrolo[2,3-d]pyrimidin-7-yl)methyl)pyridin-2-yl)-N-methylmethanesulfonamide;
7-((2-(difluoromethoxy)pyridin-3-yl)methyl)-N-(4-(piperidin-4-yl)phenyl)-7H-pyrrolo[2,3-d]pyrimidin-2-amine;
(5-((5,5-dimethyl-7-(pyridin-2-yl)-6,7-dihydro-5H-pyrrolo[2,3-d]pyrimidin-2-yl)amino)-1H-indol-3-yl)methanol;
N-(3-((dimethylamino)methyl)-1H-indol-5-yl)-5,5-dimethyl-7-(pyridin-2-yl)-6,7-dihydro-5H-pyrrolo[2,3-d]pyrimidin-2-amine;
N-(3-isopropyl-1H-indol-5-yl)-5,5-dimethyl-7-(pyridin-2-yl)-6,7-dihydro-5H-pyrrolo[2,3-d]pyrimidin-2-amine;
N5-(5,5-dimethyl-7-(pyridin-2-yl)-6,7-dihydro-5H-pyrrolo[2,3-d]pyrimidin-2-yl)-N3,N3-dimethyl-1H-indole-3,5-diamine;
5,5-dimethyl-N-(3-(prop-1-en-2-yl)-1H-indol-5-yl)-7-(pyridin-2-yl)-6,7-dihydro-5H-pyrrolo[2,3-d]pyrimidin-2-amine;
N-methyl-N-(2-((2-((4-(piperazin-1-yl)phenyl)amino)-7H-pyrrolo[2,3-d]pyrimidin-7-yl)methyl)cyclohexyl)methanesulfonamide;
N-methyl-N-(1-((2-((4-(piperazin-1-yl)phenyl)amino)-7H-pyrrolo[2,3-d]pyrimidin-7-yl)methyl)cyclohexyl)methanesulfonamide;
4-(2-((4-(piperazin-1-yl)phenyl)amino)-7H-pyrrolo[2,3-d]pyrimidin-7-yl)-3,4-dihydro-1H-pyrido[2,3-c][1,2]thiazine 2,2-dioxide;
4-(2-((4-(piperazin-1-yl)phenyl)amino)-7H-pyrrolo[2,3-d]pyrimidin-7-yl)-3,4-dihydro-1H-pyrido[2,3-c][1,2,6]thiadiazine 2,2-dioxide;
N-(4-(piperazin-1-yl)phenyl)-7-((1,2,3,4-tetrahydroquinolin-8-yl)sulfonyl)-7H-pyrrolo[2,3-d]pyrimidin-2-amine;
8-((2-((4-(piperazin-1-yl)phenyl)amino)-7H-pyrrolo[2,3-d]pyrimidin-7-yl)sulfonyl)-2,3-dihydroquinolin-4(1H)-one;
N-(4-(piperazin-1-yl)phenyl)-7-((5,6,7,8-tetrahydroquinolin-8-yl)sulfonyl)-7H-pyrrolo[2,3-d]pyrimidin-2-amine;
(S)—N-(4-(dimethylamino)-2-((2-((4-(3-methylpiperazin-1-yl)phenyl)amino)-7H-pyrrolo[2,3-d]pyrimidin-7-yl)methyl)phenyl)-N-methylmethanesulfonamide;
N-(5-chloro-3-((2-((4-(4-(dimethylamino)piperidin-1-yl)phenyl)amino)-7H-pyrrolo[2,3-d]pyrimidin-7-yl)methyl)pyridin-2-yl)-N-methylmethanesulfonamide;
N-(4-chloro-2-((2-((4-(1-methylpiperidin-4-yl)phenyl)amino)-7H-pyrrolo[2,3-d]pyrimidin-7-yl)methyl)phenyl)-N-methylmethanesulfonamide;
N-(2-(2-(5,5-dimethyl-2-((4-(4-methylpiperazin-1-yl)phenyl)amino)-5,6-dihydro-7H-pyrrolo[2,3-d]pyrimidin-7-yl)-2-oxoethyl)phenyl)-N-methylmethanesulfonamide;
5,5-difluoro-N-(4-(4-methylpiperazin-1-yl)phenyl)-7-(quinolin-8-ylsulfonyl)-6,7-dihydro-5H-pyrrolo[2,3-d]pyrimidin-2-amine;
N-(5-chloro-3-((2-((4-(piperazin-1-yl)phenyl)amino)-7H-pyrrolo[2,3-d]pyrimidin-7-yl)methyl)pyridin-2-yl)-N-methylmethanesulfonamide;
(S)—N-(5-chloro-3-((2-((4-(3-methylpiperazin-1-yl)phenyl)amino)-7H-pyrrolo[2,3-d]pyrimidin-7-yl)methyl)pyridin-2-yl)-N-methylmethanesulfonamide;
(S)—N-(5-(dimethylamino)-3-((2-((4-(3-methylpiperazin-1-yl)phenyl)amino)-7H-pyrrolo[2,3-d]pyrimidin-7-yl)methyl)pyridin-2-yl)-N-methylmethanesulfonamide;
N-(4-(dimethylamino)-2-((2-((4-(4-(2-hydroxyethyl)piperazin-1-yl)phenyl)amino)-7H-pyrrolo[2,3-d]pyrimidin-7-yl)methyl)phenyl)-N-methylmethanesulfonamide;
N-(5-fluoro-3-((2-((4-(piperazin-1-yl)phenyl)amino)-7H-pyrrolo[2,3-d]pyrimidin-7-yl)methyl)pyridin-2-yl)-N-methylmethanesulfonamide;
N-methyl-N-(3-((2-((4-(piperazin-1-yl)phenyl)amino)-7H-pyrrolo[2,3-d]pyrimidin-7-yl)methyl)-5-(trifluoromethyl)pyridin-2-yl)methanesulfonamide;
(S)—N-methyl-N-(3-((2-((4-(3-methylpiperazin-1-yl)phenyl)amino)-7H-pyrrolo[2,3-d]pyrimidin-7-yl)methyl)-5-(trifluoromethyl)pyridin-2-yl)methanesulfonamide;
(R)—N-(5-fluoro-3-((2-((4-(3-methylpiperazin-1-yl)phenyl)amino)-7H-pyrrolo[2,3-d]pyrimidin-7-yl)methyl)pyridin-2-yl)-N-methylmethanesulfonamide;
(R)—N-methyl-N-(3-((2-((4-(3-methylpiperazin-1-yl)phenyl)amino)-7H-pyrrolo[2,3-d]pyrimidin-7-yl)methyl)-5-(trifluoromethyl)pyridin-2-yl)methanesulfonamide;
N-(4-(dimethylamino)-2-((2-((4-(piperidin-4-yl)phenyl)amino)-7H-pyrrolo[2,3-d]pyrimidin-7-yl)methyl)phenyl)-N-methylmethanesulfonamide;
N-(4-fluoro-2-((2-((4-(1-methylpiperidin-4-yl)phenyl)amino)-7H-pyrrolo[2,3-d]pyrimidin-7-yl)methyl)phenyl)-N-methylmethanesulfonamide;
N-methyl-N-(2-methyl-6-((2-((4-(piperazin-1-yl)phenyl)amino)-7H-pyrrolo[2,3-d]pyrimidin-7-yl)methyl)phenyl)methanesulfonamide;
N-(2-chloro-6-((2-((4-(piperazin-1-yl)phenyl)amino)-7H-pyrrolo[2,3-d]pyrimidin-7-yl)methyl)phenyl)-N-methylmethanesulfonamide;
N-(2-cyano-6-((2-((4-(piperazin-1-yl)phenyl)amino)-7H-pyrrolo[2,3-d]pyrimidin-7-yl)methyl)phenyl)-N-methylmethanesulfonamide;
N-(2-fluoro-6-((2-((4-(piperazin-1-yl)phenyl)amino)-7H-pyrrolo[2,3-d]pyrimidin-7-yl)methyl)phenyl)-N-methylmethanesulfonamide;
N-(2-(dimethylamino)-6-((2-((4-(piperazin-1-yl)phenyl)amino)-7H-pyrrolo[2,3-d]pyrimidin-7-yl)methyl)phenyl)-N-methylmethanesulfonamide;
N-(2-((2-((4-(4-hydroxypiperidin-1-yl)phenyl)amino)-7H-pyrrolo[2,3-d]pyrimidin-7-yl)methyl)-4-methylphenyl)-N-methylmethanesulfonamide;
N-(2-((2-((4-(4-(dimethylamino)piperidin-1-yl)-3-methoxyphenyl)amino)-7H-pyrrolo[2,3-d]pyrimidin-7-yl)methyl)-4-methylphenyl)-N-methylmethanesulfonamide;
N-(2-((2-((4-(4-(dimethylamino)piperidin-1-yl)-3-fluorophenyl)amino)-7H-pyrrolo[2,3-d]pyrimidin-7-yl)methyl)-4-methylphenyl)-N-methylmethanesulfonamide;
N-(3-((2-((3-fluoro-4-(piperazin-1-yl)phenyl)amino)-7H-pyrrolo[2,3-d]pyrimidin-7-yl)methyl)-5-methoxypyridin-2-yl)-N-methylmethanesulfonamide;
N-(2-((2-((4-(4-(dimethylamino)piperidin-1-yl)-3-fluorophenyl)amino)-7H-pyrrolo[2,3-d]pyrimidin-7-yl)methyl)-4-methoxyphenyl)-N-methylmethanesulfonamide;
N-(2-((2-((4-(4-hydroxypiperidin-1-yl)phenyl)amino)-7H-pyrrolo[2,3-d]pyrimidin-7-yl)methyl)-4-methoxyphenyl)-N-methylmethanesulfonamide;
N-(5-fluoro-3-((2-((3-methoxy-4-(piperazin-1-yl)phenyl)amino)-7H-pyrrolo[2,3-d]pyrimidin-7-yl)methyl)pyridin-2-yl)-N-methylmethanesulfonamide;
N-(4-fluoro-2-((2-((4-(4-hydroxypiperidin-1-yl)phenyl)amino)-7H-pyrrolo[2,3-d]pyrimidin-7-yl)methyl)phenyl)-N-methylmethanesulfonamide;
N-methyl-N-(3-((2-((4-(piperazin-1-yl)phenyl)amino)-7H-pyrrolo[2,3-d]pyrimidin-7-yl)methyl)pyrazin-2-yl)methanesulfonamide;
N-methyl-N-(3-((2-((4-(piperazine-1-carbonyl)phenyl)amino)-7H-pyrrolo[2,3-d]pyrimidin-7-yl)methyl)pyrazin-2-yl)methanesulfonamide;
N-(3-((2-((4-(4-(2-hydroxyethyl)piperazin-1-yl)phenyl)amino)-7H-pyrrolo[2,3-d]pyrimidin-7-yl)methyl)pyrazin-2-yl)-N-methylmethanesulfonamide;
N-(3-((2-((4-(4-hydroxypiperidin-1-yl)phenyl)amino)-7H-pyrrolo[2,3-d]pyrimidin-7-yl)methyl)pyrazin-2-yl)-N-methylmethanesulfonamide;
(R)—N-methyl-N-(3-((2-((4-(3-methylpiperazin-1-yl)phenyl)amino)-7H-pyrrolo[2,3-d]pyrimidin-7-yl)methyl)pyrazin-2-yl)methanesulfonamide;
N-methyl-4-((1-((3-(N-methylmethylsulfonamido)pyrazin-2-yl)methyl)-1H-pyrrolo[3,2-c]pyridin-6-yl)amino)benzamide;
N-methyl-N-(3-((6-((4-(piperazin-1-yl)phenyl)amino)-1H-pyrrolo[3,2-c]pyridin-1-yl)methyl)pyrazin-2-yl)methanesulfonamide;
(S)—N-methyl-N-(3-((6-((4-(3-methylpiperazin-1-yl)phenyl)amino)-1H-pyrrolo[3,2-c]pyridin-1-yl)methyl)pyrazin-2-yl)methanesulfonamide;
N-methyl-4-((1-((3-(N-methylmethylsulfonamido)pyrazin-2-yl)methyl)-1H-pyrazolo[4,3-c]pyridin-6-yl)amino)benzamide;
N-methyl-N-(3-((6-((4-(piperazin-1-yl)phenyl)amino)-1H-pyrazolo[4,3-c]pyridin-1-yl)methyl)pyrazin-2-yl)methanesulfonamide;
(S)—N-methyl-N-(3-((6-((4-(3-methylpiperazin-1-yl)phenyl)amino)-1H-pyrazolo[4,3-c]pyridin-1-yl)methyl)pyrazin-2-yl)methanesulfonamide;
N-methyl-4-((1-((3-(N-methylmethylsulfonamido)pyrazin-2-yl)methyl)-1H-pyrazolo[3,4-d]pyrimidin-6-yl)amino)benzamide;
N-methyl-N-(3-((6-((4-(piperazin-1-yl)phenyl)amino)-1H-pyrazolo[3,4-d]pyrimidin-1-yl)methyl)pyrazin-2-yl)methanesulfonamide;
(S)—N-methyl-N-(3-((6-((4-(3-methylpiperazin-1-yl)phenyl)amino)-1H-pyrazolo[3,4-d]pyrimidin-1-yl)methyl)pyrazin-2-yl)methanesulfonamide;
N-(5-fluoro-3-((2-((4-(piperazin-1-yl)phenyl)amino)-7H-pyrrolo[2,3-d]pyrimidin-7-yl)methyl)pyrazin-2-yl)-N-methylmethanesulfonamide;
N-(6-fluoro-3-((2-((4-(piperazin-1-yl)phenyl)amino)-7H-pyrrolo[2,3-d]pyrimidin-7-yl)methyl)pyrazin-2-yl)-N-methylmethanesulfonamide;
N-methyl-N-(5-methyl-3-((2-((4-(piperazin-1-yl)phenyl)amino)-7H-pyrrolo[2,3-d]pyrimidin-7-yl)methyl)pyrazin-2-yl)methanesulfonamide;
N-methyl-N-(6-methyl-3-((2-((4-(piperazin-1-yl)phenyl)amino)-7H-pyrrolo[2,3-d]pyrimidin-7-yl)methyl)pyrazin-2-yl)methanesulfonamide;
N-(5-methoxy-3-((2-((4-(piperazin-1-yl)phenyl)amino)-7H-pyrrolo[2,3-d]pyrimidin-7-yl)methyl)pyrazin-2-yl)-N-methylmethanesulfonamide;
N-(6-methoxy-3-((2-((4-(piperazin-1-yl)phenyl)amino)-7H-pyrrolo[2,3-d]pyrimidin-7-yl)methyl)pyrazin-2-yl)-N-methylmethanesulfonamide;
N-(5-chloro-3-((2-((4-(piperazin-1-yl)phenyl)amino)-7H-pyrrolo[2,3-d]pyrimidin-7-yl)methyl)pyrazin-2-yl)-N-methylmethanesulfonamide; and
N-(6-chloro-3-((2-((4-(piperazin-1-yl)phenyl)amino)-7H-pyrrolo[2,3-d]pyrimidin-7-yl)methyl)pyrazin-2-yl)-N-methylmethanesulfonamide.

57. The compound according to claim 1 which is a salt of an acid or base.

58. The compound according to claim 57, wherein said acid is selected from the group consisting of acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, trifluoroacetic, and camphorsulfonic.

59. The compound according to claim 57, wherein said base is selected from the group consisting of sodium, potassium, calcium, and ammonium.

60. A pharmaceutical composition comprising a compound according to any one of claims 1 to 59 and a pharmaceutically acceptable carrier.

61. A kit comprising a compound according to any one of claims 1 to 59.

62. A method for regulating the FAK pathway, Src pathway, or a combination thereof, said method comprising administering a therapeutically effective amount of a compound of any one of claims 1 to 59 to a subject in need thereof.

63. The method according to claim 62, wherein said regulation comprises inhibiting the FAK and Src pathways.

64. A method for treating a condition treatable by inhibiting the FAK pathway, Src pathway, or a combination thereof, said method comprising administering a therapeutically effective amount of a compound of any one of claims 1 to 59 to a subject in need thereof.

65. A method of inhibiting the FAK pathway, Src pathway, or a combination thereof, said method comprising administering a compound of any of claims 1 to 59 to a subject in need thereof.

66. The method according to claim 64 or 65, said method comprising inhibiting the FAK and Src pathways.

67. A method for treating a disease characterized by abnormal cellular proliferation resulting from a dysregulated FAK pathway, Src pathway, or a combination thereof, said method comprising administering of a therapeutically effective amount of a compound of any one of claims 1 to 59 to a subject in need thereof.

68. The method according to claim 67, wherein said disease is cancer.

69. The method according to claim 68, wherein said cancer is of the prostate, head, neck, eye, mouth, throat, esophagus, bronchus, larynx, pharynx, chest, bone, lung, colon, rectum, stomach, bladder, uterus, cervix, breast, ovaries, vagina, testicles, skin, thyroid, blood, lymph nodes, kidney, liver, intestines, pancreas, brain, central nervous system, adrenal gland, or skin or a leukemia.

70. The method according to claim 67, wherein said subject has at least one solid tumor.

71. A method of treating cancer, said method comprising administering a compound of any one of claims 1 to 59 to a subject in need thereof.

72. The method according to claim 71, wherein said cancer is of the prostate, head, neck, eye, mouth, throat, esophagus, bronchus, larynx, pharynx, chest, bone, lung, colon, rectum, stomach, bladder, uterus, cervix, breast, ovaries, vagina, testicles, skin, thyroid, blood, lymph nodes, kidney, liver, intestines, pancreas, brain, central nervous system, adrenal gland, or skin or a leukemia.

73. The method according to claim 71 or 72, further comprising administering a chemotherapeutic agent.

74. The method according to claim 71 or 72, further comprising administering radiation to said subject.


SEQUENCE LISTING (7 sequences)

SEQ ID NO: 1 - DNA, 4561 bp, Homo sapiens
gcgcacgcgcgcgggcccgcgccgacgcagcacggcctcgagggcgcgagcccgcgccgccgccgccgccgccggtcccggaccactgtgagcccgcggcgtgaggcgtgggaggaagcgcggctgctgtcgcccagcgccgccccgtcgtcgtctgccttcgcttcacggcgccgagccgcggtccgagcagaactggggctcccttgcatcttccagttacaaattcagtgccttctgcagtttccccagagctcctcaagaataacggaagggagaatatgacagatacctagcatctagcaaaataatggcagctgcttaccttgaccccaacttgaatcacacaccaaattcgagtactaagactcacctgggtactggtatggaacgttctcctggtgcaatggagcgagtattaaaggtctttcattattttgaaagcaatagtgagccaaccacctgggccagtattatcaggcatggagatgctactgatgtcaggggcatcattcagaagatagtggacagtcacaaagtaaagcatgtggcctgctatggattccgcctcagtcacctgcggtcagaggaggttcactggcttcacgtggatatgggcgtctccagtgtgagggagaagtatgagcttgctcacccaccagaggagtggaaatatgaattgagaattcgttatttgccaaaaggatttctaaaccagtttactgaagataagccaactttgaatttcttctatcaacaggtgaagagcgattatatgttagagatagctgatcaagtggaccaggaaattgctttgaagttgggttgtctagaaatacggcgatcatactgggagatgcggggcaatgcactagaaaagaagtctaactatgaagtattagaaaaagatgttggtttaaagcgattttttcctaagagtttactggattctgtcaaggccaaaacactaagaaaactgatccaacaaacatttagacaatttgccaaccttaatagagaagaaagtattctgaaattctttgagatcctgtctccagtctacagatttgataaggaatgcttcaagtgtgctcttggttcaagctggattatttcagtggaactggcaatcggcccagaagaaggaatcagttacctaacggacaagggctgcaatcccacacatcttgctgacttcactcaagtgcaaaccattcagtattcaaacagtgaagacaaggacagaaaaggaatgctacaactaaaaatagcaggtgcacccgagcctctgacagtgacggcaccatccctaaccattgcggagaatatggctgacctaatagatgggtactgccggctggtgaatggaacctcgcagtcatttatcatcagacctcagaaagaaggtgaacgggctttgccatcaataccaaagttggccaacagcgaaaagcaaggcatgcggacacacgccgtctctgtgtcagaaacagatgattatgctgagattatagatgaagaagatacttacaccatgccctcaaccagggattatgagattcaaagagaaagaatagaacttggacgatgtattggagaaggccaatttggagatgtacatcaaggcatttatatgagtccagagaatccagctttggcggttgcaattaaaacatgtaaaaactgtacttcggacagcgtgagagagaaatttcttcaagaagccttaacaatgcgtcagtttgaccatcctcatattgtgaagctgattggagtcatcacagagaatcctgtctggataatcatggagctgtgcacacttggagagctgaggtcatttttgcaagtaaggaaatacagtttggatctagcatctttgatcctgtatgcctatcagcttagtacagctcttgcatatctagagagcaaaagatttgtacacagggacattgctgctcggaatgttctggtgtcctcaaatgattgtgtaaaattaggagactttggattatcccgatatatggaagatagtacttactacaaagcttccaaaggaaaattgcctattaaatggatggctccagagtcaatcaattttcgacgttttacctcagctagtgacgtatggatgtttggtgtgtgtatgtgggagatactgatgcatggtgtgaagccttttcaaggagtgaagaacaatgatgtaatcggtcgaattgaaaatggggaaagattaccaatgcctccaaattgtcctcctaccctctacagccttatgacgaaatgctgggcctatgaccccagcaggcggcccaggtttactgaacttaaagctcagctcagcacaatcctggaggaagagaaggctcagcaagaagagcgcatgaggatggagtccagaagacaggccacagtgtcctgggactccggagggtctgatgaagcaccgcccaagcccagcagaccgggttatcccagtccgaggtccagcgaaggattttatcccagcccacagcacatggtacaaaccaatcattaccaggtttctggctaccctggttcacatggaatcacagccatggctggcagcatctatccaggtcaggcatctcttttggaccaaacagattcatggaatcatagacctcaggagatagcaatgtggcagcccaatgtggaggactctacagtattggacctgcgagggattgggcaagtgttgccaacccatctgatggaagagcgtctaatccgacagcaacaggaaatggaagaagatcagcgctggctggaaaaagaggaaagatttctgaaacctgatgtgagactctctcgaggcagtattgacagggaggatggaagtcttcagggtccgattggaaaccaacatatatatcagcctgtgggtaaaccagatcctgcagctccaccaaagaaaccgcctcgccctggagctcccggtcatctgggaagccttgccagcctcagcagccctgctgacagctacaacgagggtgtcaagcttcagccccaggaaatcagcccccctcctactgccaacctggaccggtcgaatgataaggtgtacgagaatgtgacgggcctggtgaaagctgtcatcgagatgtccagtaaaatccagccagccccaccagaggagtatgtccctatggtgaaggaagtcggcttggccctgaggacattattggccactgtggatgagaccattcccctcctaccagccagcacccaccgagagattgagatggcacagaagctattgaactctgacctgggtgagctcatcaacaagatgaaactggcccagcagtatgtcatgaccagcctccagcaagagtacaaaaagcaaatgctgactgctgctcacgccctggctgtggatgccaaaaacttactcgatgtcattgaccaagcaagactgaaaatgcttgggcagacgagaccacactgagcctcccctaggagcacgtcttgctaccctcttttgaagatgttctctagccttccaccagcagcgaggaattaaccctgtgtcctcagtcgccagcacttacagctccaacttttttgaatgaccatctggttgaaaaatctttctcatataagtttaaccacactttgatttgggttcattttttgttttgtttttttcaatcatgatattcagaaaaatccaggatccaaaatgtggcgtttttctaagaatgaaaattatatgtaagcttttaagcatcatgaagaacaatttatgttcacattaagatacgttctaaagggggatggccaaggggtgacatcttaattcctaaactaccttagctgcatagtggaagaggagagcatgaagcaaagaattccaggaaacccaagaggctgagaattcttttgtctaccatagaattattatccagactggaatttttgtttgttagaacacccttcagttgcaatatgctaatcccactttacaaagaatataaaagctatattttgaagacttgagttatttcagaaaaaactacagccctttttgtcttacctgccttttactttcgtgtggatatgtgaagcattgggtcgggaactagctgtagaacacaactaaaaactcatgtcttttttcacagaataatgtgccagttttttgtagcaatgttatttctcttggaagcagaaatgctttgtaccagagcacctccaaactgcattgaggagaagttccagaaccatcccctttttccatttttatataatttataaagaaagattaaagccatgttgactattttacagccactggagttaactaacccttccttgtatctgtcttcccaggagagaatgaagcaaaacaggaatttggttttcttttgatgtccagttacaccatccattctgttaattttgaaaaaatataccctccctttagtttgttgggggatataaattattctcaggaagaatataatgaactgtacagttactttgacctattaaaaaggtgttaccagtaaagttcttgttgtaatatccttaaaaaaaa

SEQ ID NO: 2 - protein, 1052 aa, Homo sapiens
MAAAYLDPNLNHTPNSSTKTHLGTGMERSPGAMERVLKVFHYFESNSEPTTWASIIRHGDATDVRGIIQKIVDSHKVKHVACYGFRLSHLRSEEVHWLHVDMGVSSVREKYELAHPPEEWKYELRIRYLPKGFLNQFTEDKPTLNFFYQQVKSDYMLEIADQVDQEIALKLGCLEIRRSYWEMRGNALEKKSNYEVLEKDVGLKRFFPKSLLDSVKAKTLRKLIQQTFRQFANLNREESILKFFEILSPVYRFDKECFKCALGSSWIISVELAIGPEEGISYLTDKGCNPTHLADFTQVQTIQYSNSEDKDRKGMLQLKIAGAPEPLTVTAPSLTIAENMADLIDGYCRLVNGTSQSFIIRPQKEGERALPSIPKLANSEKQGMRTHAVSVSETDDYAEIIDEEDTYTMPSTRDYEIQRERIELGRCIGEGQFGDVHQGIYMSPENPALAVAIKTCKNCTSDSVREKFLQEALTMRQFDHPHIVKLIGVITENPVWIIMELCTLGELRSFLQVRKYSLDLASLILYAYQLSTALAYLESKRFVHRDIAARNVLVSSNDCVKLGDFGLSRYMEDSTYYKASKGKLPIKWMAPESINFRRFTSASDVWMFGVCMWEILMHGVKPFQGVKNNDVIGRIENGERLPMPPNCPPTLYSLMTKCWAYDPSRRPRFTELKAQLSTILEEEKAQQEERMRMESRRQATVSWDSGGSDEAPPKPSRPGYPSPRSSEGFYPSPQHMVQTNHYQVSGYPGSHGITAMAGSIYPGQASLLDQTDSWNHRPQEIAMWQPNVEDSTVLDLRGIGQVLPTHLMEERLIRQQQEMEEDQRWLEKEERFLKPDVRLSRGSIDREDGSLQGPIGNQHIYQPVGKPDPAAPPKKPPRPGAPGHLGSLASLSSPADSYNEGVKLQPQEISPPPTANLDRSNDKVYENVTGLVKAVIEMSSKIQPAPPEEYVPMVKEVGLALRTLLATVDETIPLLPASTHREIEMAQKLLNSDLGELINKMKLAQQYVMTSLQQEYKKQMLTAAHALAVDAKNLLDVIDQARLKMLGQTRPH

SEQ ID NO: 3 - protein, 5 aa, Artificial Sequence
  source: /note=“Description of Artificial Sequence Synthetic peptide”
PHSCN

SEQ ID NO: 4 - protein, 5 aa, Artificial Sequence
  source: /note=“Description of Artificial Sequence Synthetic peptide”
PHSRN

SEQ ID NO: 5 - protein, 6 aa, Artificial Sequence
  source: /note=“Description of Artificial Sequence Synthetic 6xHis tag”
HHHHHH

SEQ ID NO: 6 - protein, 7 aa, Artificial Sequence
  source: /note=“Description of Artificial Sequence Synthetic peptide”
EEAYGWM

SEQ ID NO: 7 - protein, 15 aa, Artificial Sequence
  source: /note=“Description of Artificial Sequence Synthetic peptide”
EQEDEPEGDYFEWLE